(19) United States
(12) Patent Application Publication
(10) Pub. No.: US 2023/0108906 A1
(43) Pub. Date: Apr. 6, 2023

(54) SUBSTITUTED BICYCLIC AND TRICYCLIC UREAS AND AMIDES, ANALOGUES THEREOF, AND METHODS USING SAME

(71) Applicant: Arbutus Biopharma Corporation, Burnaby (CA)

(72) Inventors: Andrew G. Cole, Cranbury, NJ (US); Bruce D. Dorsey, Ambler, PA (US); Yi Fan, Doylestown, PA (US); Steven G. Kultgen, Warminster, PA (US); Eugen F. Mesaros, Wallingford, PA (US)

(21) Appl. No.: 17/784,596

(22) PCT Filed: Dec. 18, 2020
(86) PCT No.: PCT/US2020/065888

§ 371 Date: Jun. 10, 2022

Related U.S. Application Data
(60) Provisional application No. 62/951,299, filed on Dec. 20, 2019.
(60) Provisional application No. 63/036,687, filed on Jun. 9, 2020.

(51) Int. Cl. A61K 31/4741; C07D 215/38; C07D 221/18; C07D 227/087; C07D 491/052; C07D 491/153; C07D 495/14; C07D 471/04; C07D 495/04; A61K 31/4704; A61K 31/435; A61K 31/473; A61K 31/436; A61K 31/4365; A61K 31/4745; A61K 31/4743; A61K 45/06; A61K 47/54; A61P 31/20

(52) U.S. Cl. CPC A61K 31/4741; C07D 215/38; C07D 221/18; C07D 227/087; C07D 491/052; C07D 491/153; C07D 495/14; C07D 471/04; C07D 495/04; A61K 31/4704; A61K 31/435; A61K 31/473; A61K 31/436; A61K 31/4365; A61K 31/4745; A61K 31/4743; A61K 45/06; A61K 47/549; A61P 31/20; C07B 2200/07; C07B 2200/05; C07B 2200/09

(57) ABSTRACT

The present disclosure includes in one aspect substituted arylmethyl ureas, substituted heteroarylmethyl ureas, or analogues thereof, and compositions comprising the same, that can be used to treat or prevent hepatitis B virus (HBV) and/or hepatitis D virus (HDV) infections in a patient.

### CROSS REFERENCE TO RELATED APPLICATIONS

This application claims priority under 35 U.S.C. § 119(e) to U.S. Provisional Application No. 62/951,299, filed Dec. 20, 2019, and No. 63/036,687, filed Jun. 9, 2020, all of which applications are incorporated herein by reference in their entireties.

### BACKGROUND

Hepatitis B is one of the world's most prevalent diseases, being listed by National Institute of Allergy and Infectious Diseases (NIAID) as a High Priority Area of Interest. Although most individuals resolve the infection following acute symptoms, approximately 30% of cases become chronic. 350-400 million people worldwide are estimated to have chronic hepatitis B, leading to 0.5-1 million deaths per year, due largely to the development of hepatocellular carcinoma, cirrhosis and/or other complications.

A limited number of drugs are currently approved for the management of chronic hepatitis B, including two formulations of alpha-interferon (standard and pegylated) and five nucleoside/nucleotide analogues (lamivudine, adefovir, entecavir, telbivudine, and tenofovir) that inhibit hepatitis B virus (HBV) DNA polymerase. At present, the first-line treatment choices are entecavir, tenofovir and/or peg-interferon alfa-2a. However, peg-interferon alfa-2a achieves desirable serological milestones in only one third of treated patients, and is frequently associated with severe side effects. Entecavir and tenofovir are potent HBV inhibitors, but require long-term or possibly lifetime administration to continuously suppress HBV replication, and may eventually fail due to emergence of drug-resistant viruses. There is thus a pressing need for the introduction of novel, safe, and effective therapies for chronic hepatitis B.

HBV is a noncytopathic, liver tropic DNA virus belonging to Hepadnaviridae family. Pregenomic (pg) RNA is the template for reverse transcriptional replication of HBV DNA. The encapsidation of pg RNA, together with viral DNA polymerase, into a nucleocapsid is essential for the subsequent viral DNA synthesis. Inhibition of pg RNA encapsidation may block HBV replication and provide a new therapeutic approach to HBV treatment. A capsid inhibitor acts by inhibiting the expression and/or function of a capsid protein either directly or indirectly: for example, it may inhibit capsid assembly, induce formation of non-capsid polymers, promote excess capsid assembly or misdirected capsid assembly, affect capsid stabilization, and/or inhibit RNA encapsidation. A capsid inhibitor may also act by inhibiting capsid function in one or more downstream events within the replication process, such as, but not limited to, viral DNA synthesis, transport of relaxed circular DNA (rcDNA) into the nucleus, covalently closed circular DNA (cccDNA) formation, virus maturation, budding and/or release.

Clinically, inhibition of pg RNA encapsidation, or more generally inhibition of nucleocapsid assembly, may offer certain therapeutic advantages. In one aspect, inhibition of pg RNA encapsidation may complement the current medications by providing an option for a subpopulation of patients that do not tolerate or benefit from the current medications. In another aspect, based on their distinct antiviral mechanism, inhibition of pg RNA encapsidation may be effective against HBV variants resistant to the currently available DNA polymerase inhibitors. In yet another aspect, combination therapy of the pg RNA encapsidation inhibitors with DNA polymerase inhibitors may synergistically suppress HBV replication and prevent drug resistance emergence, thus offering a more effective treatment for chronic hepatitis B infection.

Hepatitis D virus (HDV) is a small circular enveloped RNA virus that can propagate only in the presence of HBV. In particular, HDV requires the HBV surface antigen protein to propagate itself. Infection with both HBV and HDV results in more severe complications compared to infection with HBV alone. These complications include a greater likelihood of experiencing liver failure in acute infections and a rapid progression to liver cirrhosis, with an increased chance of developing liver cancer in chronic infections. In combination with hepatitis B, hepatitis D has the highest mortality rate of all the hepatitis infections. The routes of transmission of HDV are similar to those for HBV. Infection is largely restricted to persons at high risk of HBV infection, particularly injecting drug users and persons receiving clotting factor concentrates.

Currently, there is no effective antiviral therapy available for the treatment of acute or chronic type D hepatitis. Interferon-alfa given weekly for 12 to 18 months is the only licensed treatment for hepatitis D. Response to this therapy is limited, as only about one-quarter of patients is serum HDV RNA undetectable 6 months post therapy.

Clinically, inhibition of pg RNA encapsidation, or more generally inhibition of nucleocapsid assembly, may offer certain therapeutic advantages for treatment of hepatitis B and/or hepatitis D. In one aspect, inhibition of pg RNA encapsidation may complement the current medications by providing an option for a subpopulation of patients that do not tolerate or benefit from the current medications. In another aspect, based on their distinct antiviral mechanism, inhibition of pg RNA encapsidation may be effective against HBV and/or HDV variants resistant to the currently available DNA polymerase inhibitors. In yet another aspect, combination therapy of the pg RNA encapsidation inhibitors with DNA polymerase inhibitors may synergistically suppress HBV and/or HDV replication and prevent drug resistance emergence, thus offering a more effective treatment for chronic hepatitis B and/or hepatis D infection.

There is thus a need in the art for the identification of novel compounds that can be used to treat and/or prevent HBV and/or HDV infection in a subject. In certain embodiments, the novel compounds inhibit HBV and/or HDV nucleocapsid assembly. In other embodiments, the novel compounds can be used in patients that are HBV and/or HBV-HDV infected, patients who are at risk of becoming HBV and/or HBV-HDV infected, and/or patients that are infected with drug-resistant HBV and/or HDV. The present disclosure addresses this need.

### BRIEF SUMMARY

The present disclosure provides a compound of formula (I), or a salt, solvate, prodrug, stereoisomer, tautomer, or isotopically labelled derivative thereof, or any mixtures thereof:

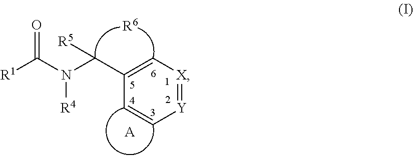

wherein R¹, R⁴, R⁵, R⁶, X, Y, and A ring are defined elsewhere herein.

The present disclosure further provides a pharmaceutical composition comprising at least one compound of the disclosure and a pharmaceutically acceptable carrier.

The present disclosure further provides a method of treating, ameliorating, and/or preventing hepatitis B virus (HBV) infection in a subject. The present disclosure further provides a method of inhibiting expression and/or function of a viral capsid protein directly or indirectly in a HBV-infected subject. In certain embodiments, the method comprises administering to the subject in need thereof a therapeutically effective amount of at least one compound of the disclosure and/or at least one pharmaceutical composition of the disclosure.

### BRIEF DESCRIPTION OF THE DRAWINGS

The drawings illustrate generally, by way of example, but not by way of limitation, various embodiments of the present application.

FIG. 1 illustrates an ORTEP drawing of Compound 72 with 50% thermal ellipsoids.

### DETAILED DESCRIPTION

The disclosure relates, in certain aspects, to the discovery of certain substituted ureas and amides that are useful to treat, ameliorate, and/or prevent hepatitis B virus (HBV) and/or hepatitis D virus (HDV) infection and related conditions in a subject. In certain non-limiting embodiments, the compounds of the disclosure are viral capsid inhibitors.

### Definitions

As used herein, each of the following terms has the meaning associated with it in this section. Unless defined otherwise, all technical and scientific terms used herein generally have the same meaning as commonly understood by one of ordinary skill in the art to which this disclosure belongs. Generally, the nomenclature used herein and the laboratory procedures in animal pharmacology, pharmaceutical science, separation science, and organic chemistry are those well-known and commonly employed in the art. It should be understood that the order of steps or order for performing certain actions is immaterial, so long as the present teachings remain operable. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the application, where an element or component is said to be included in and/or selected from a list of recited elements or components, it should be understood that the element or component can be any one of the recited elements or components and can be selected from a group consisting of two or more of the recited elements or components.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.”

As used herein, the term “about” will be understood by persons of ordinary skill in the art and will vary to some extent on the context in which it is used. As used herein, “about” when referring to a measurable value such as an amount, a temporal duration, and the like, is meant to encompass variations of ±20%, ±10%, ±5%, ±1%, or 0.1% from the specified value, as such variations are appropriate to perform the disclosed methods.

As used herein, the term “alkenyl,” employed alone or in combination with other terms, means, unless otherwise stated, a stable monounsaturated or diunsaturated straight chain or branched chain hydrocarbon group having the stated number of carbon atoms. Examples include vinyl, propenyl (or allyl), crotyl, isopentenyl, butadienyl, 1,3-pentadienyl, 1,4-pentadienyl, and the higher homologs and isomers. A functional group representing an alkene is exemplified by —CH₂—CH═CH₂.

As used herein, the term “alkoxy” employed alone or in combination with other terms means, unless otherwise stated, an alkyl group having the designated number of carbon atoms, as defined elsewhere herein, connected to the rest of the molecule via an oxygen atom, such as, for example, methoxy, ethoxy, 1-propoxy, 2-propoxy (or isopropoxy) and the higher homologs and isomers. A specific example is (C₁-C₃)alkoxy, such as, but not limited to, ethoxy and methoxy.

As used herein, the term “alkyl” by itself or as part of another substituent means, unless otherwise stated, a straight or branched chain hydrocarbon having the number of carbon atoms designated (i.e., C₁-C₁₀ means one to ten carbon atoms) and includes straight, branched chain, or cyclic substituent groups. Examples include methyl, ethyl, propyl, isopropyl, butyl, isobutyl, tert-butyl, pentyl, neopentyl, hexyl, and cyclopropylmethyl. A specific embodiment is (C₁-C₆)alkyl, such as, but not limited to, ethyl, methyl, isopropyl, isobutyl, n-pentyl, n-hexyl, and cyclopropylmethyl.

As used herein, the term “alkynyl” employed alone or in combination with other terms means, unless otherwise stated, a stable straight chain or branched chain hydrocarbon group with a triple carbon-carbon bond, having the stated number of carbon atoms. Non-limiting examples include ethynyl and propynyl, and the higher homologs and isomers. The term “propargylic” refers to a group exemplified by —CH₂—C≡CH. The term “homopropargylic” refers to a group exemplified by —CH₂CH₂—C≡CH.

As used herein, the term “aromatic” refers to a carbocycle or heterocycle with one or more polyunsaturated rings and having aromatic character, i.e., having (4n+2) delocalized 71 (pi) electrons, where ‘n’ is an integer.

As used herein, the term “aryl” employed alone or in combination with other terms means, unless otherwise stated, a carbocyclic aromatic system containing one or more rings (typically one, two or three rings) wherein such rings may be attached together in a pendent manner, such as a biphenyl, or may be fused, such as naphthalene. Examples include phenyl, anthracyl and naphthyl. Aryl groups also include, for example, phenyl or naphthyl rings fused with one or more saturated or partially saturated carbon rings (e.g., bicyclo[4.2.0]octa-1,3,5-trienyl, or indanyl), which can be substituted at one or more carbon atoms of the aromatic and/or saturated or partially saturated rings.

As used herein, the term “aryl-(C₁-C₆)alkyl” refers to a functional group wherein a one-to-six carbon alkylene chain is attached to an aryl group, e.g., —CH₂CH₂-phenyl or —CH₂-phenyl (or benzyl). Specific examples are aryl-CH₂— and aryl-CH(CH₃)—. The term “substituted aryl-(C₁-C₆)alkyl” refers to an aryl-(C₁-C₆)alkyl functional group in which the aryl group is substituted. A specific example is substituted aryl(CH₂)—. Similarly, the term “heteroaryl-(C₁-C₆)alkyl” refers to a functional group wherein a one-to-three carbon alkylene chain is attached to a heteroaryl group, e.g., —CH₂CH₂-pyridyl. A specific example is heteroaryl-(CH₂)—. The term “substituted heteroaryl-(C₁-C₆)alkyl” refers to a heteroaryl-(C₁-C₆)alkyl functional group in which the heteroaryl group is substituted. A specific example is substituted heteroaryl-(CH₂)—.

In one aspect, the terms “co-administered” and “co-administration” as relating to a subject refer to administering to the subject a compound and/or composition of the disclosure along with a compound and/or composition that may also treat or prevent a disease or disorder contemplated herein. In certain embodiments, the co-administered compounds and/or compositions are administered separately, or in any kind of combination as part of a single therapeutic approach. The co-administered compound and/or composition may be formulated in any kind of combinations as mixtures of solids and liquids under a variety of solid, gel, and liquid formulations, and as a solution.

As used herein, the term “cycloalkyl” by itself or as part of another substituent refers to, unless otherwise stated, a cyclic chain hydrocarbon having the number of carbon atoms designated (i.e., C₃-C₆ refers to a cyclic group comprising a ring group consisting of three to six carbon atoms) and includes straight, branched chain or cyclic substituent groups. Examples of (C₃-C₆)cycloalkyl groups are cyclopropyl, cyclobutyl, cyclopentyl and cyclohexyl. Cycloalkyl rings can be optionally substituted. Non-limiting examples of cycloalkyl groups include: cyclopropyl, 2-methyl-cyclopropyl, cyclopropenyl, cyclobutyl, 2,3-dihydroxycyclobutyl, cyclobutenyl, cyclopentyl, cyclopentenyl, cyclopentadienyl, cyclohexyl, cyclohexenyl, cycloheptyl, cyclooctanyl, decalinyl, 2,5-dimethylcyclopentyl, 3,5-dichlorocyclohexyl, 4-hydroxycyclohexyl, 3,3,5-trimethylcyclohex-1-yl, octahydropentalenyl, octahydro-1H-indenyl, 3a,4,5,6,7,7a-hexahydro-3H-inden-4-yl, decahydroazulenyl; bicyclo[6.2.0]decanyl, decahydronaphthalenyl, and dodecahydro-1H-fluorenyl. The term “cycloalkyl” also includes bicyclic hydrocarbon rings, non-limiting examples of which include, bicyclo[2.1.1]hexanyl, bicyclo[2.2.1]heptanyl, bicyclo[3.1.1]heptanyl, 1,3-dimethyl[2.2.1]heptan-2-yl, bicyclo[2.2.2]octanyl, and bicyclo[3.3.3]undecanyl.

As used herein, a “disease” is a state of health of a subject wherein the subject cannot maintain homeostasis, and wherein if the disease is not ameliorated then the subject's health continues to deteriorate.

As used herein, a “disorder” in a subject is a state of health in which the subject is able to maintain homeostasis, but in which the subject's state of health is less favorable than it would be in the absence of the disorder. Left untreated, a disorder does not necessarily cause a further decrease in the subject's state of health.

As used herein, the term “halide” refers to a halogen atom bearing a negative charge.

The halide anions are fluoride (F⁻), chloride (Cl⁻), bromide (Br⁻), and iodide (I⁻).

As used herein, the term “halo” or “halogen” alone or as part of another substituent refers to, unless otherwise stated, a fluorine, chlorine, bromine, or iodine atom.

As used herein, the term “heteroalkenyl” by itself or in combination with another term refers to, unless otherwise stated, a stable straight or branched chain monounsaturated or diunsaturated hydrocarbon group consisting of the stated number of carbon atoms and one or two heteroatoms selected from the group consisting of O, N, and S, and wherein the nitrogen and sulfur atoms may optionally be oxidized and the nitrogen heteroatom may optionally be quaternized. Up to two heteroatoms may be placed consecutively. Examples include —CH═CH—O—CH₃, —CH═CH—CH₂—OH, —CH₂—CH═N—OCH₃, —CH═CH—N(CH₃)—CH₃, and —CH₂—CH═CH—CH₂—SH.

As used herein, the term “heteroalkyl” by itself or in combination with another term refers to, unless otherwise stated, a stable straight or branched chain alkyl group consisting of the stated number of carbon atoms and one or two heteroatoms selected from the group consisting of O, N, and S, and wherein the nitrogen and sulfur atoms may be optionally oxidized and the nitrogen heteroatom may be optionally quaternized. The heteroatom(s) may be placed at any position of the heteroalkyl group, including between the rest of the heteroalkyl group and the fragment to which it is attached, as well as attached to the most distal carbon atom in the heteroalkyl group. Examples include: —OCH₂CH₂CH₃, —CH₂CH₂CH₂OH, —CH₂CH₂NHCH₃, —CH₂SCH₂CH₃, and —CH₂CH₂S(═O)CH₃. Up to two heteroatoms may be consecutive, such as, for example, —CH₂NH—OCH₃, or —CH₂CH₂SSCH₃.

As used herein, the term “heteroaryl” or “heteroaromatic” refers to a heterocycle having aromatic character. A polycyclic heteroaryl may include one or more rings that are partially saturated. Examples include tetrahydroquinoline and 2,3-dihydrobenzofuryl.

As used herein, the term “heterocycle” or “heterocyclyl” or “heterocyclic” by itself or as part of another substituent refers to, unless otherwise stated, an unsubstituted or substituted, stable, mono- or multi-cyclic heterocyclic ring system that comprises carbon atoms and at least one heteroatom selected from the group consisting of N, O, and S, and wherein the nitrogen and sulfur heteroatoms may be optionally oxidized, and the nitrogen atom may be optionally quaternized. The heterocyclic system may be attached, unless otherwise stated, at any heteroatom or carbon atom that affords a stable structure. A heterocycle may be aromatic or non-aromatic in nature. In certain embodiments, the heterocycle is a heteroaryl.

Examples of non-aromatic heterocycles include monocyclic groups such as aziridine, oxirane, thiirane, azetidine, oxetane, thietane, pyrrolidine, pyrroline, imidazoline, pyrazolidine, dioxolane, sulfolane, 2,3-dihydrofuran, 2,5-dihydrofuran, tetrahydrofuran, thiophane, piperidine, 1,2,3,6-tetrahydropyridine, 1,4-dihydropyridine, piperazine, morpholine, thiomorpholine, pyran, 2,3-dihydropyran, tetrahydropyran, 1,4-dioxane, 1,3-dioxane, homopiperazine, homopiperidine, 1,3-dioxepane, 4,7-dihydro-1,3-dioxepin, and hexamethyleneoxide.

Examples of heteroaryl groups include pyridyl, pyrazinyl, pyrimidinyl (such as, but not limited to, 2- and 4-pyrimidinyl), pyridazinyl, thienyl, furyl, pyrrolyl, imidazolyl, thiazolyl, oxazolyl, pyrazolyl, isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl, and 1,3,4-oxadiazolyl.

Examples of polycyclic heterocycles include indolyl (such as, but not limited to, 3-, 4-, 5-, 6- and 7-indolyl), indolinyl, quinolyl, tetrahydroquinolyl, isoquinolyl (such as, but not limited to, 1- and 5-isoquinolyl), 1,2,3,4-tetrahydroisoquinolyl, cinnolinyl, quinoxalinyl (such as, but not limited to, 2- and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (such as, but not limited to, 3-, 4-, 5-, 6- and 7-benzofuryl), 2,3-dihydrobenzofuryl, 1,2-benzisoxazolyl, benzothienyl (such as, but not limited to, 3-, 4-, 5-, 6-, and 7-benzothienyl), benzoxazolyl, benzothiazolyl (such as, but not limited to, 2-benzothiazolyl and 5-benzothiazolyl), purinyl, benzimidazolyl, benztriazolyl, thioxanthinyl, carbazolyl, carbolinyl, acridinyl, pyrrolizidinyl, and quinolizidinyl.

The aforementioned listing of heterocyclyl and heteroaryl moieties is intended to be representative and not limiting.

As used herein, the term “pharmaceutical composition” or “composition” refers to a mixture of at least one compound useful within the disclosure with a pharmaceutically acceptable carrier. The pharmaceutical composition facilitates administration of the compound to a subject.

As used herein, the term “pharmaceutically acceptable” refers to a material, such as a carrier or diluent, which does not abrogate the biological activity or properties of the compound useful within the disclosure, and is relatively non-toxic, i.e., the material may be administered to a subject without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the disclosure within or to the subject such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the disclosure, and not injurious to the subject. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the disclosure, and are physiologically acceptable to the subject. Supplementary active compounds may also be incorporated into the compositions. The “pharmaceutically acceptable carrier” may further include a pharmaceutically acceptable salt of the compound useful within the disclosure. Other additional ingredients that may be included in the pharmaceutical compositions used in the practice of the disclosure are known in the art and described, for example in Remington's Pharmaceutical Sciences (Genaro, Ed., Mack Publishing Co., 1985, Easton, Pa.), which is incorporated herein by reference.

As used herein, the language “pharmaceutically acceptable salt” refers to a salt of the administered compound prepared from pharmaceutically acceptable non-toxic acids and/or bases, including inorganic acids, inorganic bases, organic acids, inorganic bases, solvates (including hydrates) and clathrates thereof.

As used herein, a “pharmaceutically effective amount,” “therapeutically effective amount,” or “effective amount” of a compound is that amount of compound that is sufficient to provide a beneficial effect to the subject to which the compound is administered.

The term “prevent,” “preventing,” or “prevention” as used herein means avoiding or delaying the onset of symptoms associated with a disease or condition in a subject that has not developed such symptoms at the time the administering of an agent or compound commences. Disease, condition and disorder are used interchangeably herein.

By the term “specifically bind” or “specifically binds” as used herein is meant that a first molecule preferentially binds to a second molecule (e.g., a particular receptor or enzyme), but does not necessarily bind only to that second molecule.

As used herein, the terms “subject” and “individual” and “patient” can be used interchangeably and may refer to a human or non-human mammal or a bird. Non-human mammals include, for example, livestock and pets, such as ovine, bovine, porcine, canine, feline and murine mammals. In certain embodiments, the subject is human.

As used herein, the term “substituted” refers to that an atom or group of atoms has replaced hydrogen as the substituent attached to another group.

As used herein, the term “substituted alkyl,” “substituted cycloalkyl,” “substituted alkenyl,” or “substituted alkynyl” refers to alkyl, cycloalkyl, alkenyl, or alkynyl, as defined elsewhere herein, substituted by one, two or three substituents independently selected from the group consisting of halogen, —OH, alkoxy, tetrahydro-2-H-pyranyl, —NH₂, —NH(C₁-C₆ alkyl), —N(C₁-C₆ alkyl)₂, 1-methyl-imidazol-2-yl, pyridin-2-yl, pyridin-3-yl, pyridin-4-yl, —C(═O)OH, —C(═O)O(C₁-C₆)alkyl, trifluoromethyl, —C≡N, —C(═O)NH₂, —C(═O)NH(C₁-C₆)alkyl, —C(═O)N((C₁-C₆)alkyl)₂, —SO₂NH₂, —SO₂NH(C₁-C₆ alkyl), —SO₂N(C₁-C₆ alkyl)₂, —C(═NH)NH₂, and —NO₂, in certain embodiments containing one or two substituents independently selected from halogen, —OH, alkoxy, —NH₂, trifluoromethyl, —N(CH₃)₂, and —C(═O)OH, in certain embodiments independently selected from halogen, alkoxy and —OH.

Examples of substituted alkyls include, but are not limited to, 2,2-difluoropropyl, 2-carboxycyclopentyl and 3-chloropropyl.

For aryl, aryl-(C₁-C₃)alkyl and heterocyclyl groups, the term “substituted” as applied to the rings of these groups refers to any level of substitution, namely mono-, di-, tri-, tetra-, or penta-substitution, where such substitution is permitted. The substituents are independently selected, and substitution may be at any chemically accessible position. In certain embodiments, the substituents vary in number between one and four. In other embodiments, the substituents vary in number between one and three. In yet another embodiments, the substituents vary in number between one and two. In yet other embodiments, the substituents are independently selected from the group consisting of C₁-C₆ alkyl, —OH, C₁-C₆ alkoxy, halogen, amino, acetamido and nitro. As used herein, where a substituent is an alkyl or alkoxy group, the carbon chain may be branched, straight or cyclic.

Unless otherwise noted, when two substituents are taken together to form a ring having a specified number of ring atoms (e.g., R² and R³ taken together with the nitrogen to which they are attached to form a ring having from 3 to 7 ring members), the ring can have carbon atoms and optionally one or more (e.g., 1 to 3) additional heteroatoms independently selected from nitrogen, oxygen, or sulfur. The ring can be saturated or partially saturated, and can be optionally substituted.

Whenever a term or either of their prefix roots appear in a name of a substituent the name is to be interpreted as including those limitations provided herein. For example, whenever the term “alkyl” or “aryl” or either of their prefix roots appear in a name of a substituent (e.g., arylalkyl, alkylamino) the name is to be interpreted as including those limitations given elsewhere herein for “alkyl” and “aryl” respectively.

In certain embodiments, substituents of compounds are disclosed in groups or in ranges. It is specifically intended that the description include each and every individual subcombination of the members of such groups and ranges. For example, the term “C₁₋₆ alkyl” is specifically intended to individually disclose C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₈, C₃-C₄, C₄-C₆, C₄-C₅, and C₅-C₆ alkyl.

The terms “treat,” “treating” and “treatment,” as used herein, means reducing the frequency or severity with which symptoms of a disease or condition are experienced by a subject by virtue of administering an agent or compound to the subject.

Certain abbreviations used herein follow: cccDNA, covalently closed circular DNA; DAD, diode array detector; DCE, 1,2-dichloroethane; DCM, dichloromethane; DIEA or DIPEA, diisopropylethylamine; DMF, N,N-dimethylformamide; DMSO, dimethylsulfoxide; EtOAc, ethyl acetate; HATU, hexafluorophosphate azabenzotriazole tetramethyl uronium; HBsAg, HBV surface antigen; HBV, hepatitis B virus; HDV, hepatitis D virus; HPLC, high pressure liquid chromatography; IPA, isopropanol (2-propanol); LCMS, liquid chromatography mass spectrometry; LG, leaving group; NARTI or NRTI, reverse-transcriptase inhibitor; NBS, N-bromosuccinimide; NMR, Nuclear Magnetic Resonance; NtARTI or NtRTI, nucleotide analog reverse-transcriptase inhibitor; PCC, pyridinium chlorochromate; pg RNA, pregenomic RNA; rcDNA, relaxed circular DNA; RT, retention time; sAg, surface antigen; SFC, supercritical fluid chromatography; STAB, sodium triacetoxyborohydride; TFA, trifluoroacetic acid; THF, tetrahydrofuran; TLC, thin layer chromatography; TMSOTf, trimethylsilyl trifluoromethylsulfonate.

Ranges: throughout this disclosure, various aspects of the present disclosure can be presented in a range format. It should be understood that the description in range format is merely for convenience and brevity and should not be construed as an inflexible limitation on the scope of the present disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise. This applies regardless of the breadth of the range.

### Compounds

The disclosure includes a compound of formula (I), or a salt, solvate, prodrug, isotopically labelled derivative, stereoisomer (such as, in a non-limiting example, an enantiomer or diastereoisomer, and/or any mixtures thereof, such as, in a non-limiting example, mixtures in any proportions of enantiomers and/or diastereoisomers thereof), tautomer and any mixtures thereof, and/or geometric isomer and any mixtures thereof:

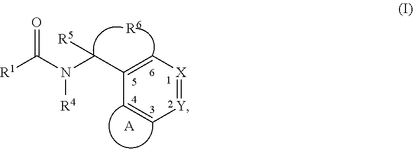

wherein:

X, Y, and the bond between X and Y are such that:
X is NR⁷, Y is C(═O), and the bond between X and Y is a single bond, or
X is N, Y is CR¹⁰, and the bond between X and Y is a double bond,

A ring

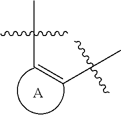

is selected from the group consisting of:

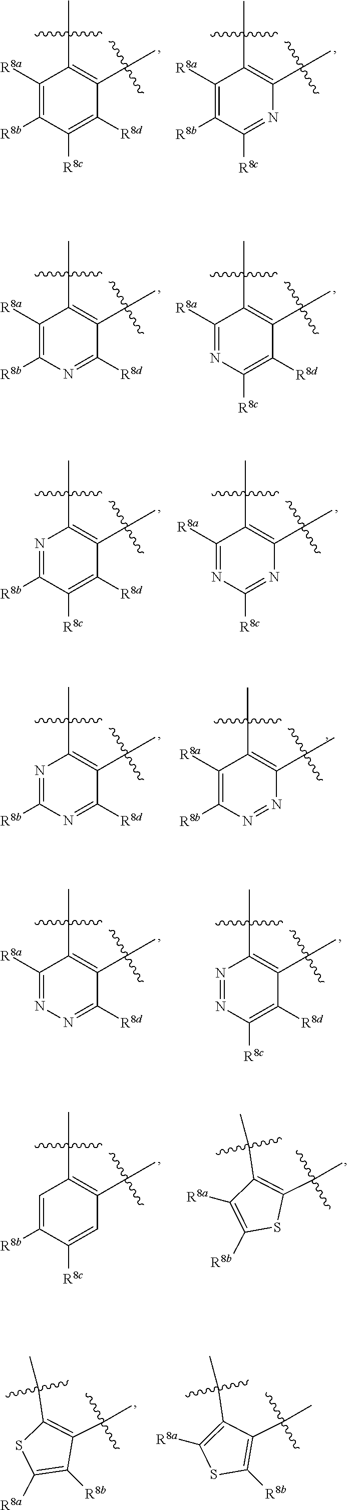

(wherein there is no bridgehead double bond),

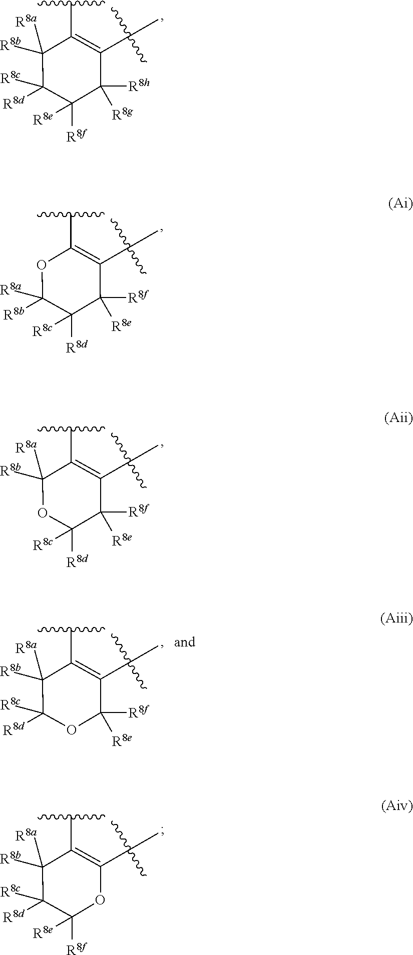

wherein:
   in (Ai) R^{8a} and R^{8b} optionally combine with the carbon atom to which they are attached to form carbonyl (—(C═O)—);
   in (Aii) R^{8a} and R^{8b}, or R^{8c} and R^{8d}, optionally combine with the carbon atom to which they are attached to form carbonyl (—(C═O)—);
   in (Aiii) R^{8c} and R^{8d}, or R^{8e} and R^{8f}, optionally combine with the carbon atom to which they are attached to form carbonyl (—(C═O)—);
   in (Aiv) R^{8e} and R^{8f} optionally combine with the carbon atom to which they are attached to form carbonyl (—(C═O)—);
or the A ring is absent, position 3 of the pyridin-2-one ring is substituted with R^{8a}, and position 4 of the pyridin-2-one ring is substituted with R^{8b};

R¹ is selected from the group consisting of —NR²R³ and

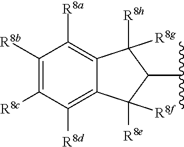

optionally substituted isoindolin-2-yl);

R² is selected from the group consisting of optionally substituted C₃-C₈ cycloalkyl, optionally substituted phenyl, optionally substituted benzyl, optionally substituted heteroaryl, and —(CH₂)(optionally substituted heteroaryl);

R³ is selected from the group consisting of H and C₁-C₆ alkyl;

R⁴ is selected from the group consisting of H, C₁-C₆ alkyl, and C₃-C₈ cycloalkyl, wherein the alkyl or cycloalkyl is optionally substituted with at least one selected from the group consisting of C₁-C₆ alkyl, C₃-C₈ cycloalkyl, halogen, cyano, —OH, C₁-C₆ alkoxy, C₃-C₈ cycloalkoxy, C₁-C₆ haloalkoxy, C₃-C₈ halocycloalkoxy, optionally substituted phenyl, optionally substituted heteroaryl, optionally substituted heterocyclyl, —C(═O)OR⁹, —OC(═O)R⁹, —SR⁹, —S(═O)R⁹, —S(═O)₂R⁹, —S(═O)₂NR⁹R⁹, —N(R⁹)S(═O)₂R⁹, —N(R⁹)C(═O)R⁹, —C(═O)NR⁹R⁹, and —NR⁹R⁹;

R⁵ is selected from the group consisting of H and optionally substituted C₁-C₆ alkyl;

R⁶ is —(CH₂)ₚ-Q-(CH₂)_{q}—,
wherein p and q are independently 0, 1, 2, or 3, and
Q is a bond (absent), —O—, —OCH(OH)—, —CH(OH)O—, —S—, —S(═O)—, —S(═O)₂—, —NR¹¹, —CH(OH)—, —C(═O)—, —C(═O)O—, or —OC(═O)—,
wherein p and q are selected such that:
   2≤(p+q)≤4 if Q is a bond (absent),
   1≤(p+q)≤3 if Q is —O—, S—, —S(═O)—, —S(═O)₂—, —NR¹¹, —CH(OH)—, or —C(═O)—,
   0≤(p+q)≤2 if Q is —C(═O)O—, —OC(═O)—, —OCH(OH)—, or —CH(OH)O—, and
wherein each CH₂ in R⁶ is optionally independently substituted with one or two methyl groups;

R⁷ is selected from the group consisting of H, optionally substituted C₁-C₆ alkyl, and optionally substituted C₃-C₈ cycloalkyl;

each occurrence of R^{8a}, R^{8b}, R^{8c}, R^{8d}, R^{8e}, R^{8f}, R^{8g}, and R^{8h} is independently selected from the group consisting of H, halogen, —CN, optionally substituted C₁-C₆ alkyl, optionally substituted C₃-C₈ cycloalkyl, optionally substituted C₁-C₆ alkoxy, optionally substituted C₃-C₈ cycloalkoxy, heterocyclyl, heteroaryl, —S(optionally substituted C₁-C₆ alkyl), —SO(optionally substituted C₁-C₆ alkyl), —SO₂(optionally substituted C₁-C₆ alkoxy), —C(═O)OH, —C(═O)O(optionally substituted C₁-C₆ alkyl), —C(═O)O(optionally substituted C₃-C₈ cycloalkyl), —O(optionally substituted C₁-C₆ alkyl), —O(optionally substituted C₃-C₈ cycloalkyl), —NH₂, —NH(optionally substituted C₁-C₆ alkyl), —NH(optionally substituted C₃-C₈ cycloalkyl), —N(optionally substituted C₁-C₆ alkyl)(optionally substituted C₁-C₆ alkyl), —N(optionally substituted C₃-C₈ cycloalkyl)(optionally substituted C₃-C₈ cycloalkyl), —N(optionally substituted C₁-C₆ alkyl)(optionally substituted C₃-C₈ cycloalkyl), —C(═O)NH₂, —C(═O)NH(optionally substituted C₁-C₆ alkyl), —C(═O)NH(optionally substituted C₃-C₈ cycloalkyl), —C(═O)N(optionally substituted C₁-C₆ alkyl)(optionally substituted C₁-C₆ alkyl), —C(═O)N(optionally substituted C₃-C₈ cycloalkyl)(optionally substituted C₃-C₈ cycloalkyl), and —C(═O)N(optionally substituted C₁-C₆ alkyl)(optionally substituted C₃-C₈ cycloalkyl;

each occurrence of R⁹ is independently selected from the group consisting of H, optionally substituted C₁-C₆ alkyl, optionally substituted C₃-C₈ cycloalkyl, optionally substituted phenyl, and optionally substituted hetereoaryl;

R¹⁰ is selected from the group consisting of H, halogen, —CN, optionally substituted C₁-C₆ alkyl, optionally substituted C₃-C₈ cycloalkyl, optionally substituted C₁-C₆ alkoxy, optionally substituted C₃-C₈ cycloalkoxy, heterocyclyl, heteroaryl, —S(optionally substituted C₁-C₆ alkyl), —SO(optionally substituted C₁-C₆ alkyl), —SO₂(optionally substituted C₁-C₆ alkyl), —C(═O)OH, —C(═O)O(optionally substituted C₁-C₆ alkyl), —C(═O)O(optionally substituted C₃-C₈ cycloalkyl), —O(optionally substituted C₁-C₆ alkyl), —O(optionally substituted C₃-C₈ cycloalkyl), —NH₂, —NH(optionally substituted C₁-C₆ alkyl), —NH(optionally substituted C₃-C₈ cycloalkyl), —N(optionally substituted C₁-C₆ alkyl)(optionally substituted C₁-C₆ alkyl), —N(optionally substituted C₃-C₈ cycloalkyl)(optionally substituted C₃-C₈ cycloalkyl), —N(optionally substituted C₁-C₆ alkyl)(optionally substituted C₃-C₈ cycloalkyl), —C(═O)NH₂, —C(═O)NH(optionally substituted C₁-C₆ alkyl), —C(═O)NH(optionally substituted C₃-C₈ cycloalkyl), —C(═O)N(optionally substituted C₁-C₆ alkyl)(optionally substituted C₁-C₆ alkyl), —C(═O)N(optionally substituted C₃-C₈ cycloalkyl)(optionally substituted C₃-C₈ cycloalkyl), and —C(═O)N(optionally substituted C₁-C₆ alkyl)(optionally substituted C₃-C₈ cycloalkyl;

R¹¹ is selected from the group consisting of H, optionally substituted C₁-C₆ alkyl, optionally substituted C₃-C₈ cycloalkyl, optionally substituted phenyl, optionally substituted heteroaryl, and optionally substituted C₁-C₆ acyl.

In certain embodiments, the compound of formula (I) is

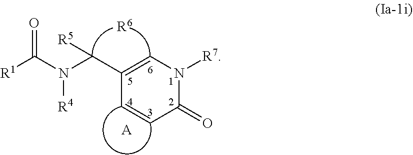

In certain embodiments, the compound of formula (I) is

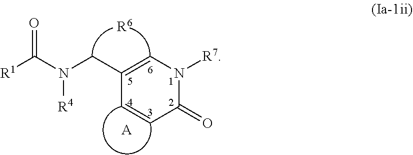

In certain embodiments, the compound of formula (I) is

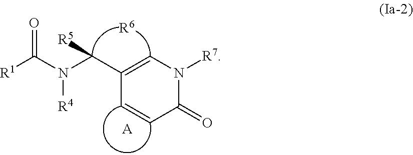

In certain embodiments, the compound of formula (I) is

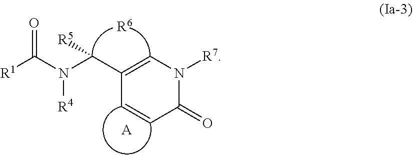

In certain embodiments, the compound of formula (I) is

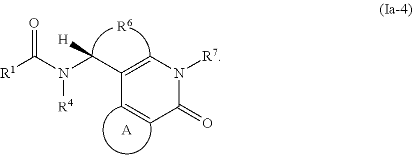

In certain embodiments, the compound of formula (I) is

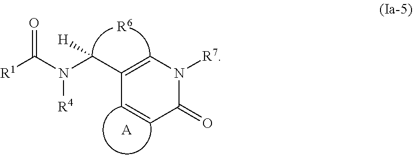

In certain embodiments, the compound of formula (I) is

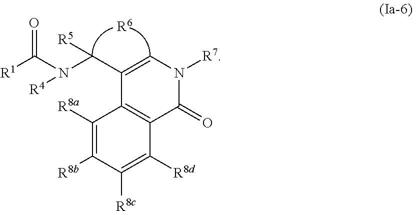

In certain embodiments, the compound of formula (I) is

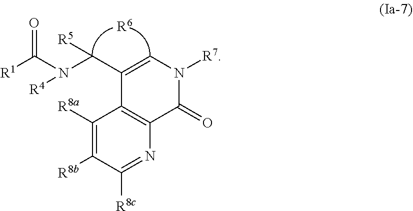

In certain embodiments, the compound of formula (I) is

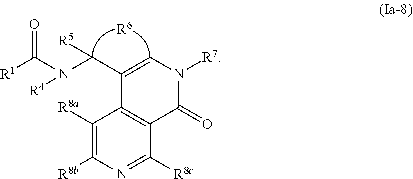

In certain embodiments, the compound of formula (I) is

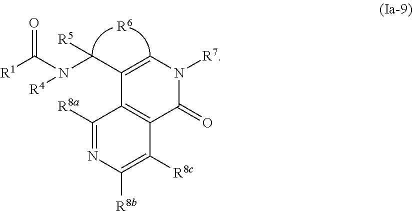

In certain embodiments, the compound of formula (I) is

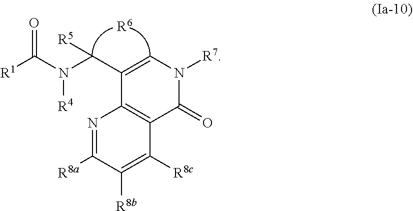

In certain embodiments, the compound of formula (I) is

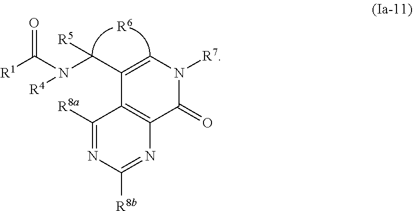

In certain embodiments, the compound of formula (I) is

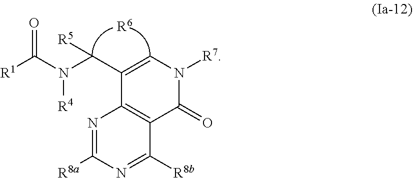

In certain embodiments, the compound of formula (I) is

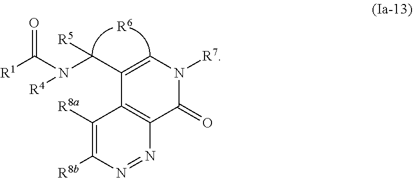

In certain embodiments, the compound of formula (I) is

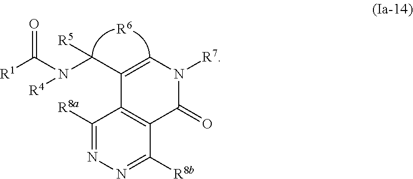

In certain embodiments, the compound of formula (I) is

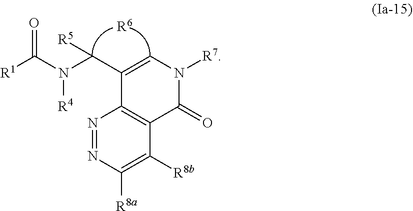

In certain embodiments, the compound of formula (I) is

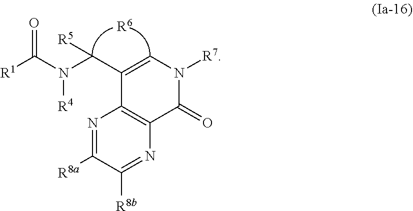

In certain embodiments, the compound of formula (I) is

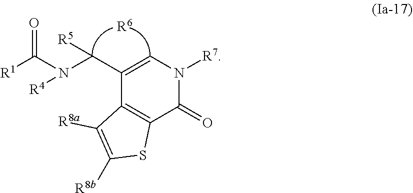

In certain embodiments, the compound of formula (I) is

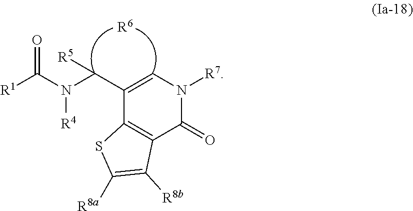

In certain embodiments, the compound of formula (I) is

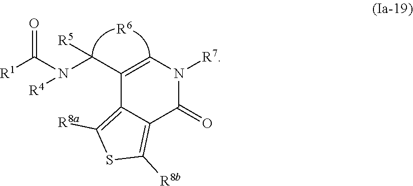

In certain embodiments, the compound of formula (I) is

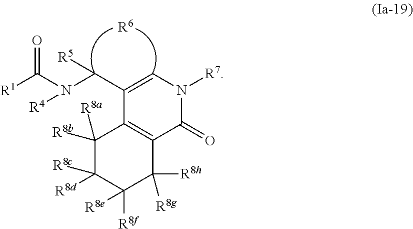

In certain embodiments, the compound of formula (I) is

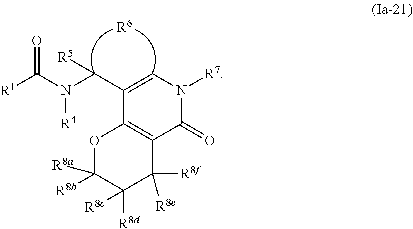

In certain embodiments, the compound of formula (I) is

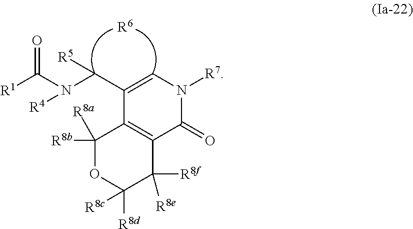

In certain embodiments, the compound of formula (I) is

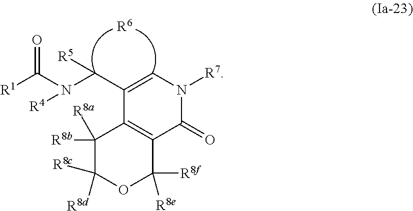

In certain embodiments, the compound of formula (I) is

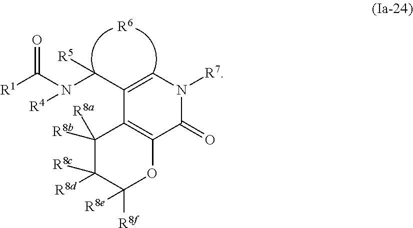

In certain embodiments, the compound of formula (I) is

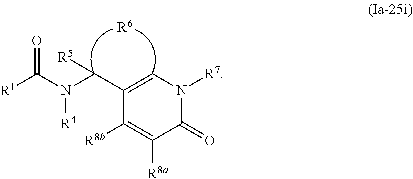

In certain embodiments, the compound of formula (I) is

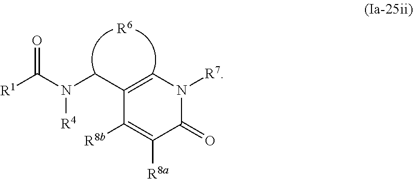

In certain embodiments, the compound of formula (I) is

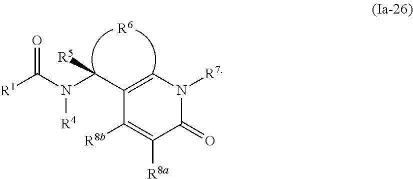

In certain embodiments, the compound of formula (I) is

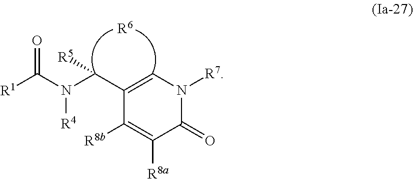

In certain embodiments, the compound of formula (I) is

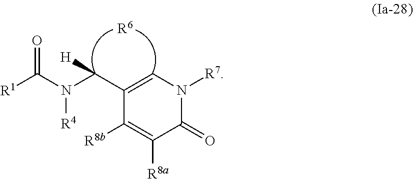

In certain embodiments, the compound of formula (I) is

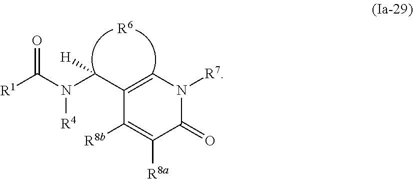

In certain embodiments, the compound of formula (I) is

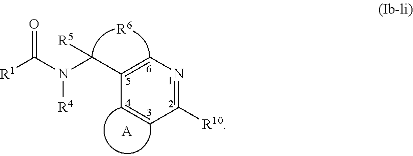

In certain embodiments, the compound of formula (I) is

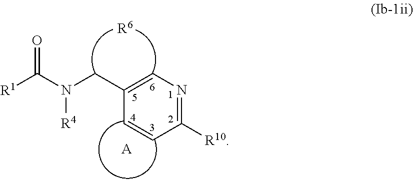

In certain embodiments, the compound of formula (I) is

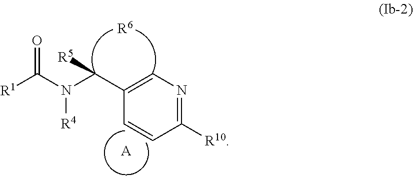

In certain embodiments, the compound of formula (I) is

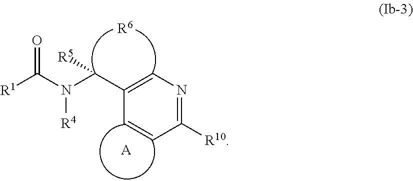

In certain embodiments, the compound of formula (I) is

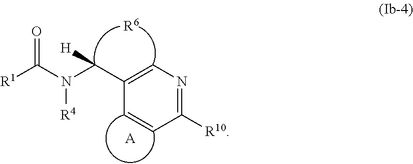

In certain embodiments, the compound of formula (I) is

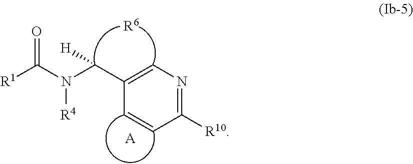

In certain embodiments, the compound of formula (I) is

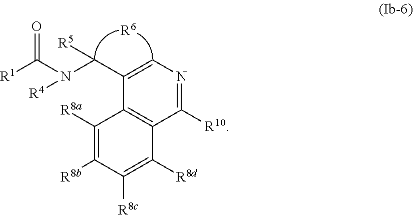

In certain embodiments, the compound of formula (I) is

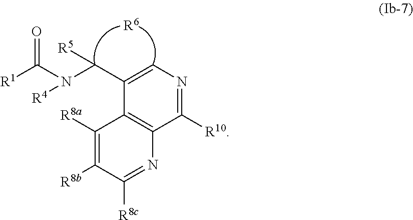

In certain embodiments, the compound of formula (I) is

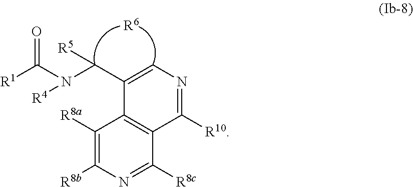

In certain embodiments, the compound of formula (I) is

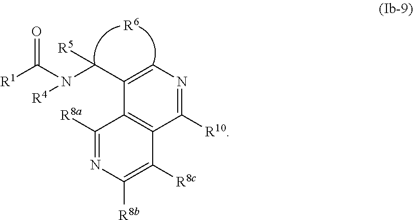

In certain embodiments, the compound of formula (I) is

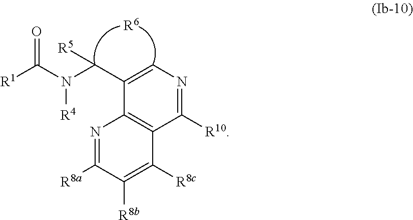

In certain embodiments, the compound of formula (I) is

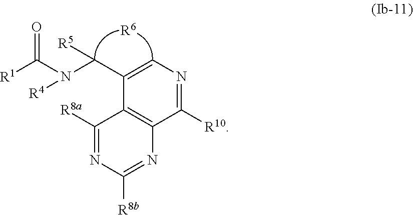

In certain embodiments, the compound of formula (I) is

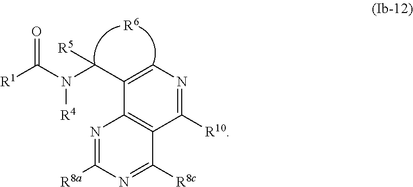

In certain embodiments, the compound of formula (I) is

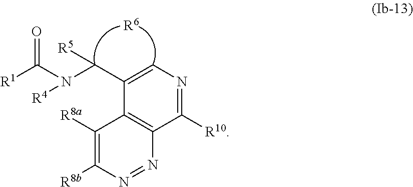

In certain embodiments, the compound of formula (I) is

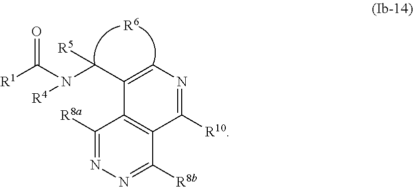

In certain embodiments, the compound of formula (I) is

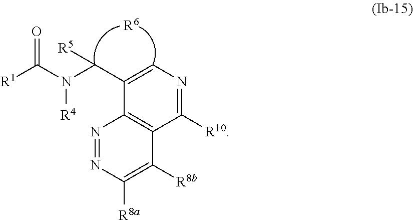

In certain embodiments, the compound of formula (I) is

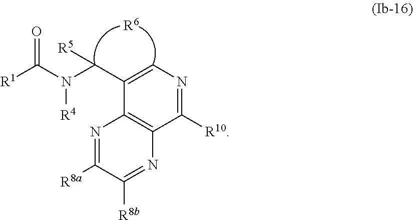

In certain embodiments, the compound of formula (I) is

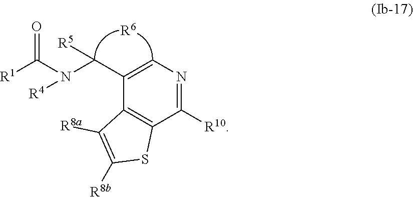

In certain embodiments, the compound of formula (I) is

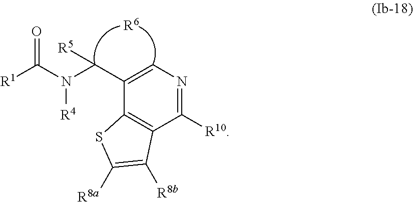

In certain embodiments, the compound of formula (I) is

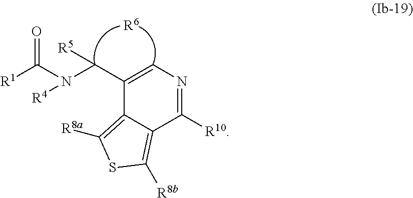

In certain embodiments, the compound of formula (I) is

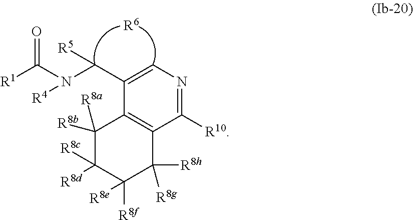

In certain embodiments, the compound of formula (I) is

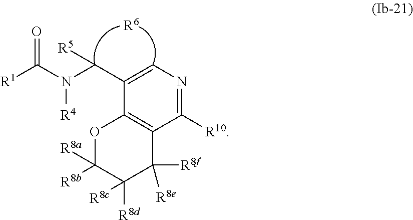

In certain embodiments, the compound of formula (I) is

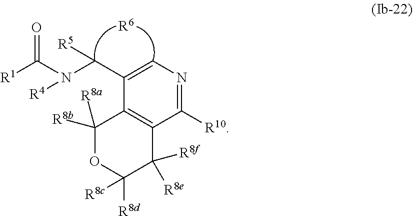

In certain embodiments, the compound of formula (I) is

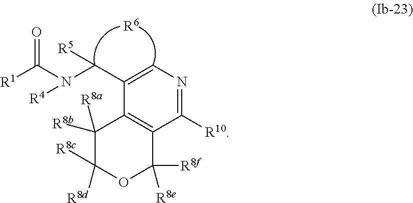

In certain embodiments, the compound of formula (I) is

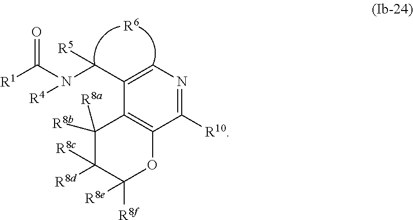

In certain embodiments, the compound of formula (I) is

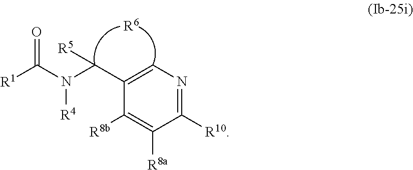

In certain embodiments, the compound of formula (I) is

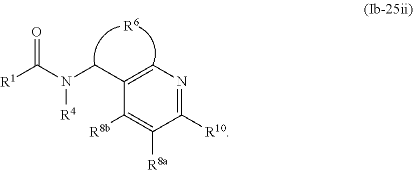

In certain embodiments, the compound of formula (I) is

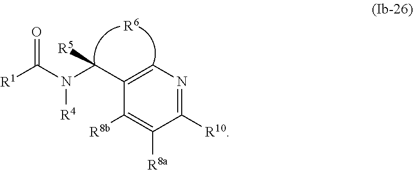

In certain embodiments, the compound of formula (I) is

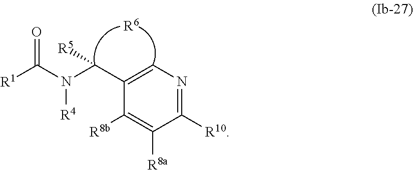

In certain embodiments, the compound of formula (I) is

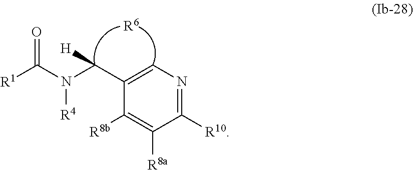

In certain embodiments, the compound of formula (I) is

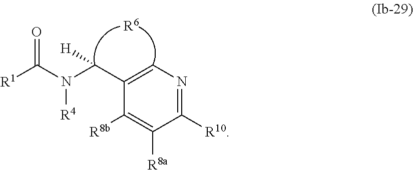

In the compound of formula (I), and in any other structure disclosed herein and/or comprised within (I), the divalent R⁶ group and the carbon atoms to which this group is attached form the following B ring:

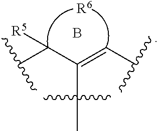

In certain embodiments, Q is a bond and the B group is

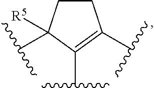

wherein each CH₂ in the B ring is optionally independently substituted with one or two methyl groups. In certain embodiments, Q is a bond and the B group is

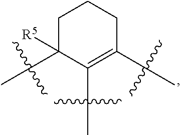

wherein each CH₂ in the B ring is optionally independently substituted with one or two methyl groups. In certain embodiments, Q is a bond and the B ring is

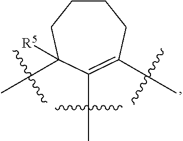

wherein each CH₂ in the B ring is optionally independently substituted with one or two methyl groups.

In certain embodiments, Q is O and the B ring is

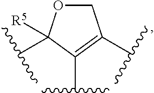

wherein the CH₂ in the B ring is optionally substituted with one or two methyl groups. In certain embodiments, Q is S and the B ring is

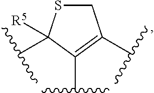

wherein the CH₂ in the B ring is optionally substituted with one or two methyl groups. In certain embodiments, Q is S═O and the B ring is

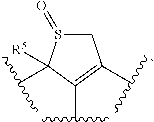

wherein the CH₂ in the B ring is optionally substituted with one or two methyl groups. In certain embodiments, Q is S(═O)₂ and the B ring is

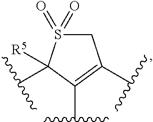

wherein the CH₂ in the B ring is optionally substituted with one or two methyl groups. In certain embodiments, Q is NR¹¹ and the B ring is

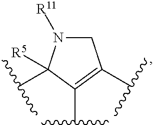

wherein the CH₂ in the B ring is optionally substituted with one or two methyl groups. In certain embodiments, Q is CH(OH) and the B ring is

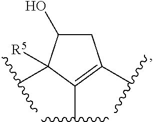

wherein the CH₂ in the B ring is optionally substituted with one or two methyl groups. In certain embodiments, Q is C═O and the B ring is

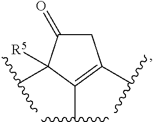

wherein the CH₂ in the B ring is optionally substituted with one or two methyl groups.

In certain embodiments, Q is O and the B ring is

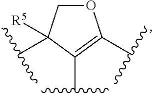

wherein the CH₂ in the B ring is optionally substituted with one or two methyl groups. In certain embodiments, Q is S and the B ring is

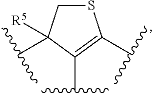

wherein the CH₂ in the B ring is optionally substituted with one or two methyl groups. In certain embodiments, Q is S═O and the B ring is

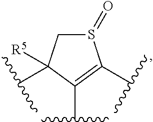

wherein the CH₂ in the B ring is optionally substituted with one or two methyl groups. In certain embodiments, Q is S(═O)₂ and the B ring is

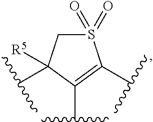

wherein the CH₂ in the B ring is optionally substituted with one or two methyl groups. In certain embodiments, Q is NR¹¹ and the B ring is

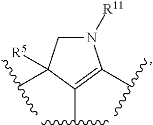

wherein the CH₂ in the B ring is optionally substituted with one or two methyl groups. In certain embodiments, Q is CH(OH) and the B ring is

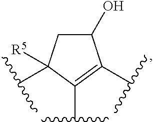

wherein the CH₂ in the B ring is optionally substituted with one or two methyl groups. In certain embodiments, Q is C═O and the B ring is

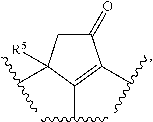

wherein the CH₂ in the B ring is optionally substituted with one or two methyl groups.

In certain embodiments, Q is O and the B ring is

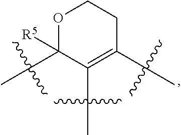

wherein each CH₂ in the B ring is optionally independently substituted with one or two methyl groups. In certain embodiments, Q is and the B ring is

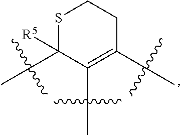

wherein each CH₂ in the B ring is optionally independently substituted with one or two methyl groups. In certain embodiments, Q is S═O and the B ring is

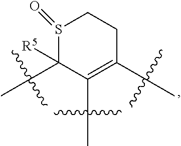

wherein each CH₂ in the B ring is optionally independently substituted with one or two methyl groups. In certain embodiments, Q is S(═O)₂ and the B ring is

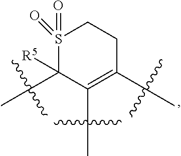

wherein each CH₂ in the B ring is optionally independently substituted with one or two methyl groups. In certain embodiments, Q is NR¹¹ and the B ring is

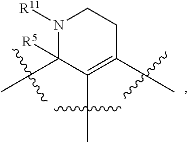

wherein each CH₂ in the B ring is optionally independently substituted with one or two methyl groups. In certain embodiments, Q is CH(OH) and the B ring is

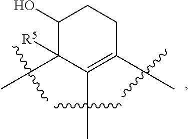

wherein each CH₂ in the B ring is optionally independently substituted with one or two methyl groups. In certain embodiments, Q is C(═O) and the B ring is

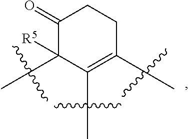

wherein each CH₂ in the B ring is optionally independently substituted with one or two methyl groups.

In certain embodiments, Q is O and the B ring is

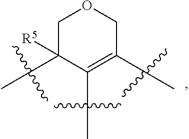

wherein each CH₂ in the B ring is optionally independently substituted with one or two methyl groups. In certain embodiments, Q is S and the B ring is

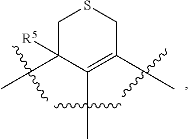

wherein each CH₂ in the B ring is optionally independently substituted with one or two methyl groups. In certain embodiments, Q is S(═O) and the B ring is

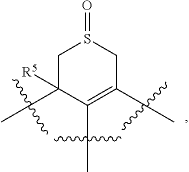

wherein each CH₂ in the B ring is optionally independently substituted with one or two methyl groups. In certain embodiments, Q is S(═O)₂ and the B ring is

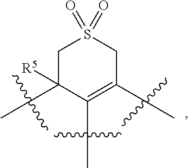

wherein each CH₂ in the B ring is optionally independently substituted with one or two methyl groups. In certain embodiments, Q is NR¹¹ and the B ring is

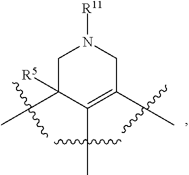

wherein each CH₂ in the B ring is optionally independently substituted with one or two methyl groups. In certain embodiments, Q is CH(OH) and the B ring is

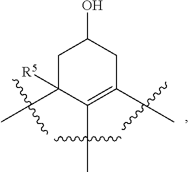

wherein each CH₂ in the B ring is optionally independently substituted with one or two methyl groups. In certain embodiments, Q is C(═O) and the B ring is

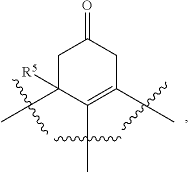

wherein each CH₂ in the B ring is optionally independently substituted with one or two methyl groups.

In certain embodiments, Q is O and the B ring is

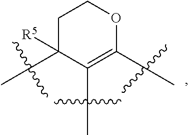

wherein each CH₂ in the B ring is optionally independently substituted with one or two methyl groups. In certain embodiments, Q is S and the B ring is

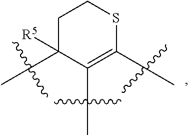

wherein each CH₂ in the B ring is optionally independently substituted with one or two methyl groups. In certain embodiments, Q is S(═O) and the B ring is

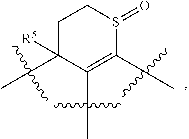

wherein each CH₂ in the B ring is optionally independently substituted with one or two methyl groups. In certain embodiments, Q is S(═O)₂ and the B ring is

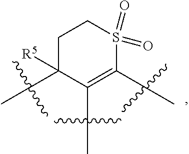

wherein each CH₂ in the B ring is optionally independently substituted with one or two methyl groups. In certain embodiments, Q is NR¹¹ and the B ring is

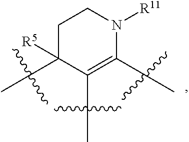

wherein each CH₂ in the B ring is optionally independently substituted with one or two methyl groups. In certain embodiments, Q is CH(OH) and the B ring is

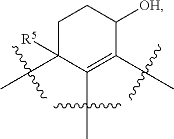

wherein each CH₂ in the B ring is optionally independently substituted with one or two methyl groups. In certain embodiments, Q is C(═O) and the B ring is

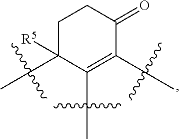

wherein each CH₂ in the B ring is optionally independently substituted with one or two methyl groups.

In certain embodiments, Q is O and the B ring is

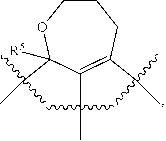

wherein each CH₂ in the B ring is optionally independently substituted with one or two methyl groups. In certain embodiments, Q is S and the B ring is

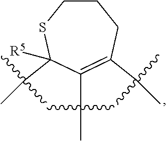

wherein each CH₂ in the B ring is optionally independently substituted with one or two methyl groups. In certain embodiments, Q is S(═O) and the B ring is

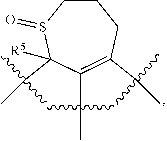

wherein each CH₂ in the B ring is optionally independently substituted with one or two methyl groups. In certain embodiments, Q is S(═O)₂ and the B ring is

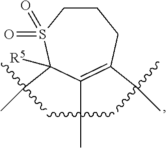

wherein each CH₂ in the B ring is optionally independently substituted with one or two methyl groups. In certain embodiments, Q is NR¹¹ and the B ring is

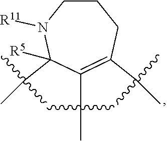

wherein each CH₂ in the B ring is optionally independently substituted with one or two methyl groups. In certain embodiments, Q is CH(OH) and the B ring is

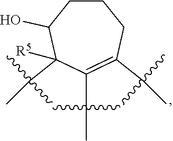

wherein each CH₂ in the B ring is optionally independently substituted with one or two methyl groups. In certain embodiments, Q is C(═O) and the B ring is

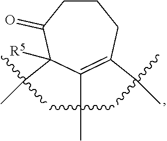

wherein each CH₂ in the B ring is optionally independently substituted with one or two methyl groups.

In certain embodiments, Q is O and the B ring is

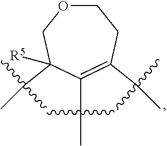

wherein each CH₂ in the B ring is optionally independently substituted with one or two methyl groups. In certain embodiments, Q is S and the B ring is

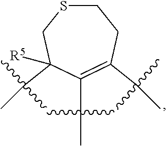

wherein each CH₂ in the B ring is optionally independently substituted with one or two methyl groups. In certain embodiments, Q is S(═O) and the B ring is

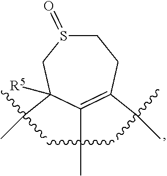

wherein each CH₂ in the B ring is optionally independently substituted with one or two methyl groups. In certain embodiments, Q is S(═O)₂ and the B ring is

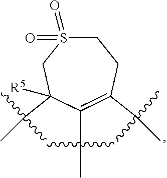

wherein each CH₂ in the B ring is optionally independently substituted with one or two methyl groups. In certain embodiments, Q is NR¹¹ and the B ring is

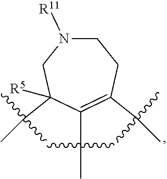

wherein each CH₂ is optionally independently substituted with one or two methyl groups. In certain embodiments, Q is CH(OH) and the B ring is

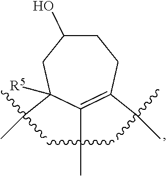

wherein each CH₂ is optionally independently substituted with one or two methyl groups. In certain embodiments, Q is C(═O) and the B ring is

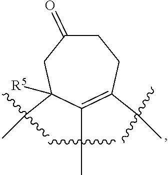

wherein each CH₂ in the B ring is optionally independently substituted with one or two methyl groups.

In certain embodiments, Q is O and the B ring is

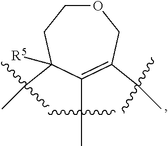

wherein each CH₂ in the B ring is optionally independently substituted with one or two methyl groups. In certain embodiments, Q is S and the B ring is

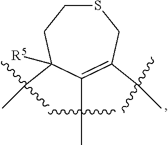

wherein each CH₂ in the B ring is optionally independently substituted with one or two methyl groups. In certain embodiments, Q is S(═O) and the B ring is

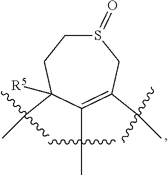

wherein each CH₂ in the B ring is optionally independently substituted with one or two methyl groups. In certain embodiments, Q is S(═O)₂ and the B ring is

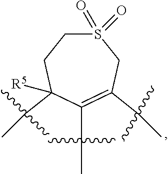

wherein each CH₂ in the B ring is optionally independently substituted with one or two methyl groups. In certain embodiments, Q is NR¹¹ and the B ring is

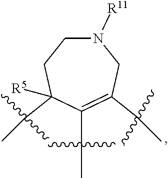

herein each CH₂ in the B ring is optionally independently substituted with one or two methyl groups. In certain embodiments, Q is CH(OH) and the B ring is

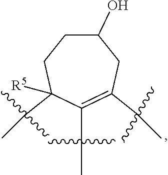

wherein each CH₂ in the B ring is optionally independently substituted with one or two methyl groups. In certain embodiments, Q is C(═O) and the B ring is

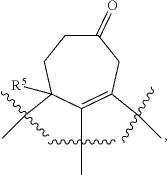

wherein each CH₂ in the B ring is optionally independently substituted with one or two methyl groups.

In certain embodiments, Q is O and the B ring is

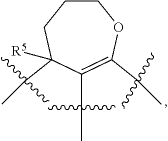

wherein each CH₂ in the B ring is optionally independently substituted with one or two methyl groups. In certain embodiments, Q is S and the B ring is

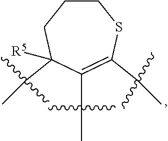

wherein each CH₂ in the B ring is optionally independently substituted with one or two methyl groups. In certain embodiments, Q is S(═O) and the B ring is

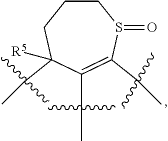

wherein each CH₂ in the B ring is optionally independently substituted with one or two methyl groups. In certain embodiments, Q is S(═O)₂ and the B ring is

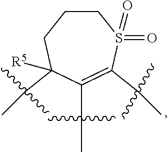

wherein each CH₂ in the B ring is optionally independently substituted with one or two methyl groups. In certain embodiments, Q is NR¹¹ and the B ring is

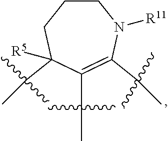

wherein each CH₂ in the B ring is optionally independently substituted with one or two methyl groups. In certain embodiments, Q is CH(OH) and the B ring is

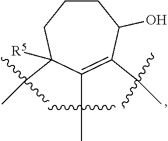

wherein each CH₂ in the B ring is optionally independently substituted with one or two methyl groups. In certain embodiments, Q is C(═O) and the B ring is

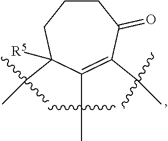

wherein each CH₂ in the B ring is optionally independently substituted with one or two methyl groups.

In certain embodiments, Q is —C(═O)O— and the B ring is

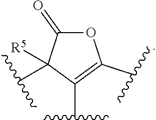

In certain embodiments, Q is —OC(═O)— and the B ring is

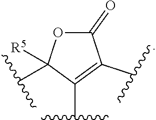

In certain embodiments, Q is —OCH(OH)— and the B ring is

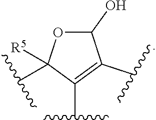

In certain embodiments, Q is —CH(OH)O— and the B ring is

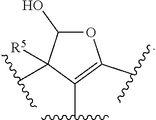

In certain embodiments, Q is —C(═O)O— and the B ring is

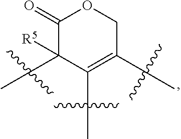

wherein the CH₂ in the B ring is optionally substituted with one or two methyl groups. In certain embodiments, Q is and the B ring is

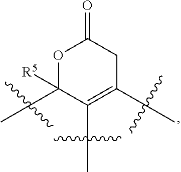

wherein the CH₂ in the B ring is optionally substituted with one or two methyl groups. In certain embodiments, Q is S═O and the B ring is

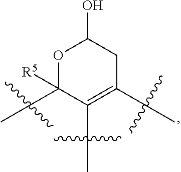

wherein the CH₂ in the B ring is optionally substituted with one or two methyl groups. In certain embodiments, Q is S(═O)₂ and the B ring is

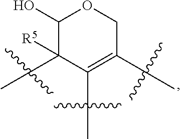

wherein the CH₂ in the B ring is optionally substituted with one or two methyl groups.

In certain embodiments, Q is —C(═O)O— and the B ring is

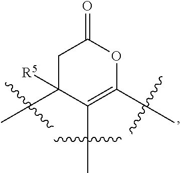

wherein the CH₂ in the B ring is optionally substituted with one or two methyl groups. In certain embodiments, Q is and the B ring is

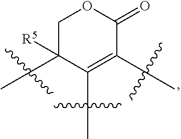

wherein the CH₂ in the B ring is optionally substituted with one or two methyl groups. In certain embodiments, Q is S═O and the B ring is

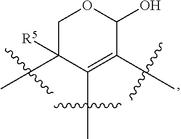

wherein the CH₂ in the B ring is optionally substituted with one or two methyl groups. In certain embodiments, Q is S(═O)₂ and the B ring is

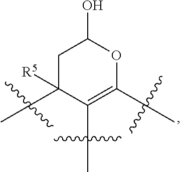

wherein the CH₂ in the B ring is optionally substituted with one or two methyl groups.

In certain embodiments, Q is —C(═O)O— and the B ring is

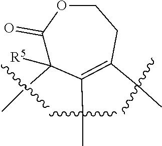

wherein each CH₂ in the B ring is optionally independently substituted with one or two methyl groups. In certain embodiments, Q is —OC(═O)— and the B ring is

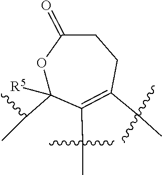

wherein each CH₂ in the B ring is optionally independently substituted with one or two methyl groups. In certain embodiments, Q is —OCH(OH)— and the B ring is

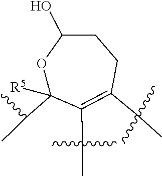

wherein each CH₂ in the B ring is optionally independently substituted with one or two methyl groups. In certain embodiments, Q is —CH(OH)O— and the B ring is

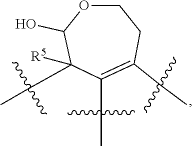

wherein each CH₂ in the B ring is optionally independently substituted with one or two methyl groups.

In certain embodiments, Q is —C(═O)O— and the B ring is

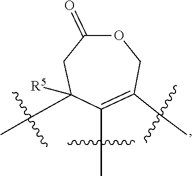

wherein each CH₂ in the B ring is optionally independently substituted with one or two methyl groups. In certain embodiments, Q is —OC(═O)— and the B ring is

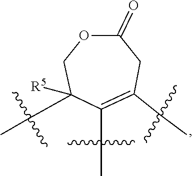

wherein each CH₂ in the B ring is optionally independently substituted with one or two methyl groups. In certain embodiments, Q is —OCH(OH)— and the B ring is

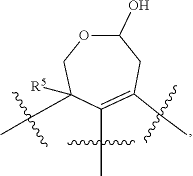

wherein each CH₂ in the B ring is optionally independently substituted with one or two methyl groups. In certain embodiments, Q is —CH(OH)O— and the B ring is

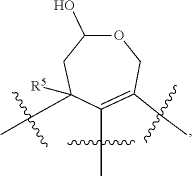

wherein each CH₂ in the B ring is optionally independently substituted with one or two methyl groups.

In certain embodiments, Q is —C(═O)O— and the B ring is

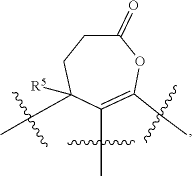

wherein each CH₂ in the B ring is optionally independently substituted with one or two methyl groups. In certain embodiments, Q is —OC(═O)— and the B ring is

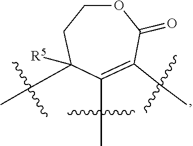

wherein each CH₂ in the B ring is optionally independently substituted with one or two methyl groups. In certain embodiments, Q is —OCH(OH)— and the B ring is

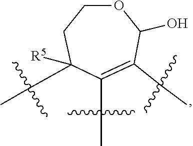

wherein each CH₂ in the B ring is optionally independently substituted with one or two methyl groups. In certain embodiments, Q is —CH(OH)O— and the B ring is

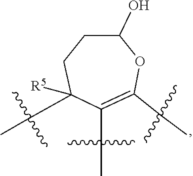

wherein each CH₂ in the B ring is optionally independently substituted with one or two methyl groups.

In certain embodiments, in any of the examples recited herein wherein the B ring comprises a hydroxyl group linked to a ring carbon, said ring carbon is optionally tertiary, being substituted with said hydroxyl group and a methyl group (i.e., the H atom exemplified elsewhere herein is replaced with a methyl group to provide —C(CH₃)(OH)—). As a non-limiting example, in certain embodiments the present disclosure contemplates that the B ring is

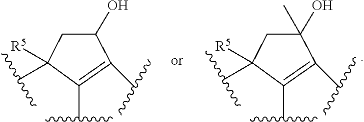

to In certain embodiments, each occurrence of alkyl, alkenyl, alkynyl, or cycloalkyl is independently optionally substituted with at least one substituent selected from the group consisting of C₁-C₆ alkyl, C₃-C₈ cycloalkyl, halogen, cyano (—CN), —OR^{a}, optionally substituted phenyl (thus yielding, in non-limiting examples, optionally substituted phenyl-(C₁-C₃ alkyl), such as, but not limited to, benzyl or substituted benzyl), optionally substituted heteroaryl, optionally substituted heterocyclyl, —C(═O)OR^{a}, —OC(═O)R^{a}, —SR^{a}, —S(═O)R^{a}, —S(═O)₂R^{a}, —S(═O)₂NR^{a}R^{a}, —N(R^{a})S(═O)₂R^{a}, —N(R^{a})C(═O)R^{a}, —C(═O)NR^{a}R^{a}, and —N(R^{a})(R^{a}), wherein each occurrence of R^{a} is independently H, optionally substituted C₁-C₆ alkyl, optionally substituted C₃-C₈ cycloalkyl, optionally substituted aryl, or optionally substituted heteroaryl, or two R^{a} groups combine with the N to which they are bound to form a heterocycle.

In certain embodiments, each occurrence of aryl or heteroaryl is independently optionally substituted with at least one substituent selected from the group consisting of C₁-C₆ alkyl, C₃-C₈ cycloalkyl, phenyl, C₁-C₆ hydroxyalkyl, (C₁-C₆ alkoxy)-C₁-C₆ alkyl, C₁-C₆ haloalkyl, C₁-C₆ haloalkoxy, halogen, —CN, —OR^{b}, —N(R^{b})(R^{b}), —NO₂, —C(═O)N(R^{b})(R^{b}), —C(═O)OR^{b}, —OC(═O)R^{b}, —SR^{b}, —S(═O)R^{b}, —S(═O)₂R^{b}, —N(R^{b})S(═O)₂R^{b}, —S(═O)₂N(R^{b})(R^{b}), acyl, and C₁-C₆ alkoxycarbonyl, wherein each occurrence of R^{b} is independently H, C₁-C₆ alkyl, or C₃-C₈ cycloalkyl, wherein in R^{b} the alkyl or cycloalkyl is optionally substituted with at least one selected from the group consisting of halogen, —OH, C₁-C₆ alkoxy, and heteroaryl; or substituents on two adjacent carbon atoms combine to form —O(CH₂)₁₋₃O—.

In certain embodiments, each occurrence of aryl or heteroaryl is independently optionally substituted with at least one substituent selected from the group consisting of C₁-C₆ alkyl, C₃-C₈ cycloalkyl, phenyl, C₁-C₆ hydroxyalkyl, (C₁-C₆ alkoxy)-C₁-C₆ alkyl, C₁-C₆ haloalkyl, C₁-C₆ haloalkoxy, halogen, —OR^{b}, —C(═O)N(R^{b})(R^{b}), —C(═O)OR^{b}, —OC(═O)R^{b}, —SR^{b}, —S(═O)R^{b}, —S(═O)₂R^{b}, and —N(R^{b})S(═O)₂R^{b}, wherein each occurrence of R^{b} is independently H, C₁-C₆ alkyl, or C₃-C₈ cycloalkyl, wherein in R^{b} the alkyl or cycloalkyl is optionally substituted with at least one selected from the group consisting of halogen, —OH, C₁-C₆ alkoxy, and heteroaryl; or substituents on two adjacent carbon atoms combine to form —O(CH₂)₁₋₃O—.

In certain embodiments, the alkyl, alkenyl, alkynyl, cycloalkyl, heteroaryl, heterocyclyl, aryl, or benzyl group is optionally independently substituted with at least one group selected from the group consisting of C₁-C₆ alkyl; C₁-C₆ alkoxy; C₁-C₆ haloalkyl; C₁-C₆ haloalkoxy; —NH₂, —NH(C₁-C₆ alkyl), —N(C₁-C₆ alkyl)(C₁-C₆ alkyl), halogen, —OH; —CN; phenoxy, —NHC(═O)H, —NHC(═O)C₁-C₆ alkyl, —C(═O)NH₂, —C(═O)NHC₁-C₆ alkyl, —C(═O)N(C₁-C₆ alkyl)(C₁-C₆ alkyl), tetrahydropyranyl, morpholinyl, —C(═O)CH₃, —C(═O)CH₂OH, —C(═O)NHCH₃, —C(═O)CH₂OMe, or an N-oxide thereof.

In certain embodiments, each occurrence of the heteroaryl is independently selected from the group consisting of quinolinyl, imidazo[1,2-a]pyridyl, pyridyl, pyrimidyl, pyrazinyl, imidazolyl, thiazolyl, pyrazolyl, isoxazolyl, oxadiazolyl (including 1,2,3-, 1,2,4-, 1,2,5-, and 1,3,4-oxadiazole), and triazolyl (such as 1,2,3-triazolyl and 1,2,4-triazolyl).

In certain embodiments, each occurrence of the heterocyclyl group is independently selected from the group consisting of tetrahydrofuranyl, tetrahydropyranyl, piperidinyl, piperazinyl, pyrrolidinyl, morpholinyl, thiomorpholinyl, 1-oxido-thiomorpholinyl, 1,1-dioxido-thiomorpholinyl, oxazolidinyl, azetidinyl, and the corresponding oxo analogues (where a methylene ring group is replaced with a carbonyl) thereof.

In certain embodiments, R¹ is —NR²R³. In certain embodiments, R¹ is

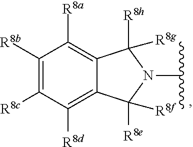

wherein each occurrence of R^{8a}, R^{8b}, R^{8c}, R^{8d}, R^{8e}, R^{8f}, R^{8g}, and R^{8h} is independently selected and is defined elsewhere herein. In certain embodiments, in R¹ at least one of R^{8b} and R^{8c} is halogen. In certain embodiments, in R¹ at least one of R^{8b} and R^{8c} is F. In certain embodiments, in R¹ at least one of R^{8b} and R^{8c} is Cl. In certain embodiments, R¹ is isoindolin-2-yl (R^{8a}-R^{8h}═H). In certain embodiments, R¹ is R^{8b}-halogen isoindolin-2-yl. In certain embodiments, R¹ is R^{8c}-halogen isoindolin-2-yl. In certain embodiments, R¹ is R^{8b}-halogen-R^{8c}-halogen isoindolin-2-yl, wherein the halogens in R^{8b} and R^{8c} are independently selected.

In certain embodiments, R² is optionally substituted C₃-C₈ cycloalkyl.

In certain embodiments, R² is selected from the group consisting of optionally substituted phenyl, optionally substituted benzyl, and —(CH₂)(optionally substituted heteroaryl), wherein the phenyl, benzyl, or heteroaryl is optionally substituted with at least one selected from the group consisting of C₁-C₆ alkyl (such as, for example, methyl, ethyl, and isopropyl), halogen (such as, for example, F, Cl, Br, and I), C₁-C₃ haloalkyl (such as, for example, monofluoromethyl, difluoromethyl, and trifluoromethyl), and —CN.

In certain embodiments, R² is selected from the group consisting of: phenyl, 3-chlorophenyl, 4-chlorophenyl, 3-fluorophenyl, 4-fluorophenyl, 3,4-difluorophenyl, 3,5-difluorophenyl, 2,4,5-trifluorophenyl, 3,4,5-trifluorophenyl, 3,4-dichlorophenyl, 3-chloro-4-fluorophenyl, 4-chloro-3-fluorophenyl, 4-chloro-3-methylphenyl, 3-chloro-4-methylphenyl, 4-fluoro-3-methylphenyl, 3-fluoro-4-methylphenyl, 4-chloro-3-methoxyphenyl, 3-chloro-4-methoxyphenyl, 4-fluoro-3-methoxyphenyl, 3-fluoro-4-methoxyphenyl, 3-trifluoromethylphenyl, 4-trifluoromethylphenyl, 3-trifluoromethyl-4-fluorophenyl, 4-trifluoromethyl-3-fluorophenyl, 3-cyanophenyl, 4-cyanophenyl, 3-cyano-4-fluorophenyl, 4-cyano-3-fluorophenyl, 3-difluoromethyl-4-fluorophenyl, 4-difluoromethyl-3-fluorophenyl, benzo[d][1,3]dioxol-5-yl, 2,3-dihydrobenzo[b][1,4]dioxin-6-yl, benzyl, 3-fluorobenzyl, 4-fluorobenzyl, 3-chlorobenzyl, 4-chlorobenzyl, 2-pyridyl, 4-methyl-2-pyridyl, 5-methyl-2-pyridyl, 6-methyl-2-pyridyl, 3-pyridyl, 2-methyl-3-pyridyl, 3-methyl-3-pyridyl, 4-pyridyl, 2-methyl-4-pyridyl, and 6-methyl-4-pyridyl. In other embodiments, R² is 3,4-difluorophenyl. In yet other embodiments, R² is 3-chloro-4-fluorophenyl. In yet other embodiments, R² is 4-chloro-3-fluorophenyl. In yet other embodiments, R² is 3-fluoro-4-methylphenyl. In yet other embodiments, R² is 4-fluoro-3-methylphenyl. In yet other embodiments, R² is 3-cyano-4-fluorophenyl. In yet other embodiments, R² is 3-difluoromethyl-4-fluorophenyl.

In certain embodiments, each occurrence of R³ is independently selected from the group consisting of H and methyl. In other embodiments, R³ is H. In yet other embodiments, R³ is methyl.

In certain embodiments, R⁴ is selected from the group consisting of H, methyl, ethyl, isopropyl, n-propyl, cyclopropyl, n-butyl, isobutyl, sec-butyl, tert-butyl, cyclobutyl, isopropylmethyl, —(CH₂)₂₋₆OH, —(CH₂)₂₋₆O(C₁-C₆ alkyl), optionally substituted benzyl, and optionally substituted phenyl.

In certain embodiments, R⁵ is selected from the group consisting of H and methyl. In other embodiments, R⁵ is H. In other embodiments, R⁵ is methyl.

In certain embodiments, p is independently 1 or 2, when Q is —O—, —S—, —S(═O)—, —S(═O)₂—, or —NR¹¹.

In certain embodiments, R⁶ is a divalent group selected from the group consisting of —CH₂CH₂—, —CH₂CH₂CH₂—, —CH₂OCH₂—, —CH₂OCH(OH)—, —CH(OH)OCH₂—, —CH₂OC(═O)—, —C(═O)OCH₂—, —CH₂SCH₂—, —CH₂S(═O)CH₂—, —CH₂S(═O)₂CH₂—, —CH₂NHCH₂—, —CH₂N(CH₃)CH₂—, —CH₂N[C(═O)CH₃]CH₂—, —CH₂N[CH₂CH₂OH]CH₂—, —CH₂CH₂CH₂CH₂—, —CH₂OCH₂CH₂—, and —CH₂CH₂OCH₂—, wherein each CH₂ group is optionally independently substituted with one or two CH₃ groups.

In certain embodiments, R⁶ is —CH₂CH₂—, wherein each CH₂ group is optionally independently substituted with one or two CH₃ groups. In certain embodiments, R⁶ is —CH₂CH₂CH₂—, wherein each CH₂ group is optionally independently substituted with one or two CH₃ groups. In certain embodiments, R⁶ is —CH₂OCH₂—, wherein each CH₂ group is optionally independently substituted with one or two CH₃ groups. In certain embodiments, R⁶ is —CH₂OCH(OH)—, wherein each CH₂ group is optionally independently substituted with one or two CH₃ groups. In certain embodiments, R⁶ is —CH(OH)OCH₂—, wherein each CH₂ group is optionally independently substituted with one or two CH₃ groups. In certain embodiments, R⁶ is —CH₂OC(═O)—, wherein each CH₂ group is optionally independently substituted with one or two CH₃ groups. In certain embodiments, R⁶ is —C(═O)OCH₂—, wherein each CH₂ group is optionally independently substituted with one or two CH₃ groups.

In certain embodiments, R⁶ is —CH₂SCH₂—, wherein each CH₂ group is optionally independently substituted with one or two CH₃ groups. In certain embodiments, R⁶ is —CH₂S(═O)CH₂—, wherein each CH₂ group is optionally independently substituted with one or two CH₃ groups. In certain embodiments, R⁶ is —CH₂S(═O)₂CH₂—, wherein each CH₂ group is optionally independently substituted with one or two CH₃ groups. In certain embodiments, R⁶ is —CH₂NHCH₂—, wherein each CH₂ group is optionally independently substituted with one or two CH₃ groups. In certain embodiments, R⁶ is —CH₂N(CH₃)CH₂—, wherein each CH₂ group is optionally independently substituted with one or two CH₃ groups. In certain embodiments, R⁶ is —CH₂N[C(═O)CH₃]CH₂—, wherein each CH₂ group is optionally independently substituted with one or two CH₃ groups. In certain embodiments, R⁶ is —CH₂N[CH₂CH₂OH]CH₂—, wherein each CH₂ group is optionally independently substituted with one or two CH₃ groups. In certain embodiments, R⁶ is —CH₂CH₂CH₂CH₂—, wherein each CH₂ group is optionally independently substituted with one or two CH₃ groups. In certain embodiments, R⁶ is —CH₂OCH₂CH₂—, wherein each CH₂ group is optionally independently substituted with one or two CH₃ groups. In certain embodiments, R⁶ is —CH₂CH₂OCH₂—, wherein each CH₂ group is optionally independently substituted with one or two CH₃ groups.

In certain embodiments, R⁶ is a divalent group selected from the group consisting of —CH₂CH₂—, —CH(CH₃)CH₂—, —CH₂CH(CH₃)—, —C(CH₃)₂CH₂—, —CH₂C(CH₃)₂—, —CH(CH₃)CH(CH₃)—, —CH(CH₃)C(CH₃)₂—, —C(CH₃)₂CH(CH₃)—, and —C(CH₃)₂C(CH₃)₂—.

In certain embodiments, R⁶ is a divalent group selected from the group consisting of —CH₂OCH₂—, —CH(CH₃)OCH₂—, —CH₂OCH(CH₃)—, —CH(CH₃)OCH(CH₃)—, —C(CH₃)₂OCH₂—, —CH₂OC(CH₃)₂—, —C(CH₃)₂OCH(CH₃)—, —CH(CH₃)OC(CH₃)₂—, and C(CH₃)₂OC(CH₃)₂—.

In certain embodiments, R⁶ is a divalent group selected from the group consisting of —CH₂CH₂CH₂—, —CH(CH₃)CH₂CH₂—, —CH₂CH(CH₃)CH₂—, —CH₂CH₂CH(CH₃)—, —CH(CH₃)CH(CH₃)CH₂—, —CH(CH₃)CH₂CH(CH₃)—, —CH₂CH(CH₃)CH(CH₃)—, —C(CH₃)₂CH₂CH₂—, —CH₂C(CH₃)₂CH₂—, —CH₂CH₂C(CH₃)₂—, —CH(CH₃)CH(CH₃)CH(CH₃)—, —C(CH₃)₂CH(CH₃)CH₂—, —C(CH₃)₂CH₂CH(CH₃)—, —CH(CH₃)C(CH₃)₂CH₂—, —CH₂C(CH₃)₂CH(CH₃)—, —CH(CH₃)CH₂C(CH₃)₂—, —CH₂CH(CH₃)C(CH₃)₂—, —C(CH₃)₂CH(CH₃)CH(CH₃)—, —C(CH₃)₂C(CH₃)₂CH₂—, —C(CH₃)₂CH₂C(CH₃)₂—, —CH(CH₃)C(CH₃)₂CH(CH₃)—, CH₂C(CH₃)₂C(CH₃)₂—, —CH(CH₃)CH(CH₃)C(CH₃)₂—, —CH(CH₃)C(CH₃)₂C(CH₃)₂—, —C(CH₃)₂CH(CH₃)C(CH₃)₂—, —C(CH₃)₂C(CH₃)₂CH(CH₃)—, and —C(CH₃)₂C(CH₃)₂C(CH₃)₂—.

In certain embodiments, R⁶ is a divalent group selected from the group consisting of —CH₂OCH₂CH₂—, —CH(CH₃)OCH₂CH₂—, —CH₂OCH(CH₃)CH₂—, —CH₂OCH₂CH(CH₃)—, —CH(CH₃)OCH(CH₃)CH₂—, —CH(CH₃)OCH₂CH(CH₃)—, —CH₂OCH(CH₃)CH(CH₃)—, —C(CH₃)₂OCH₂CH₂—, —CH₂OC(CH₃)₂CH₂—, —CH₂OCH₂C(CH₃)₂—, —CH(CH₃)OCH(CH₃)CH(CH₃)—, —C(CH₃)₂OCH(CH₃)CH₂—, —C(CH₃)₂OCH₂CH(CH₃)—, —CH(CH₃)OC(CH₃)₂CH₂—, —CH₂OC(CH₃)₂CH(CH₃)—, —CH(CH₃)OCH₂C(CH₃)₂—, —CH₂OCH(CH₃)C(CH₃)₂—, —C(CH₃)₂OCH(CH₃)CH(CH₃)—, —C(CH₃)₂OC(CH₃)₂CH₂—, —C(CH₃)₂OCH₂C(CH₃)₂—, —CH(CH₃)OC(CH₃)₂CH(CH₃)—, —CH₂OC(CH₃)₂C(CH₃)₂—, —CH(CH₃)OCH(CH₃)C(CH₃)₂—, —CH(CH₃)OC(CH₃)₂C(CH₃)₂—, —C(CH₃)₂OCH(CH₃)C(CH₃)₂—, —C(CH₃)₂OC(CH₃)₂CH(CH₃)—, and —C(CH₃)₂OC(CH₃)₂C(CH₃)₂—.

In certain embodiments, R⁶ is a divalent group selected from the group consisting of —CH₂CH₂OCH₂—, —CH(CH₃)CH₂OCH₂—, —CH₂CH(CH₃)OCH₂—, —CH₂CH₂OCH(CH₃)—, —CH(CH₃)CH(CH₃)OCH₂—, —CH(CH₃)CH₂OCH(CH₃)—, —CH₂CH(CH₃)OCH(CH₃)—, —C(CH₃)₂CH₂OCH₂—, —CH₂C(CH₃)₂OCH₂—, —CH₂CH₂OC(CH₃)₂—, —CH(CH₃) CH(CH₃)OCH(CH₃)—, —C(CH₃)₂CH(CH₃)OCH₂—, —C(CH₃)₂CH₂OCH(CH₃)—, —CH(CH₃)C(CH₃)₂OCH₂—, —CH₂C(CH₃)₂OCH(CH₃)—, —CH(CH₃)CH₂OC(CH₃)₂—, —CH₂CH(CH₃)OC(CH₃)₂—, —C(CH₃)₂CH(CH₃)OCH(CH₃)—, —C(CH₃)₂C(CH₃)₂OCH₂—, —C(CH₃)₂CH₂OC(CH₃)₂—, —CH(CH₃)C(CH₃)₂OCH(CH₃)—, —CH₂C(CH₃)₂OC(CH₃)₂—, —CH(CH₃)CH(CH₃)OC(CH₃)₂—, —CH(CH₃)C(CH₃)₂OC(CH₃)₂—, —C(CH₃)₂CH(CH₃)OC(CH₃)₂—, —C(CH₃)₂C(CH₃)₂OCH(CH₃)—, and —C(CH₃)₂C(CH₃)₂OC(CH₃)₂—.

In certain embodiments, R⁷ is H. In other embodiments, R⁷ is methyl. In yet other embodiments, R⁷ is ethyl. In yet other embodiments, R⁷ is 1-(2,2,2-trifluoroethyl). In yet other embodiments, R⁷ is 1-propyl. In yet other embodiments, R⁷ is isopropyl. In yet other embodiments, R⁷ is cyclopropyl. In yet other embodiments, R⁷ is 1-(2-hydroxy)ethyl. In yet other embodiments, R⁷ is 1-(2-methoxy)ethyl. In yet other embodiments, R⁷ is 1-(3-hydroxy)propyl. In yet other embodiments, R⁷ is 1-(3-methoxy)propyl. In yet other embodiments, R⁷ is triazolylmethyl.

In certain embodiments, R¹⁰ is H. In other embodiments, R¹⁰ is methoxy. In yet other embodiments, R¹⁰ is ethoxy. In yet other embodiments, R¹⁰ is methyl. In yet other embodiments, R¹⁰ is ethyl. In yet other embodiments, R¹⁰ is 2-hydroxyethoxy. In yet other embodiments, R¹⁰ is amino. In yet other embodiments, R¹⁰ is methylamino. In yet other embodiments, R¹⁰ is ethylamino. In yet other embodiments, R¹⁰ is dimethylamino. In yet other embodiments, R¹⁰ is (2-hydroxyethyl)amino. In yet other embodiments, R¹⁰ is 2-aminoethyl)amino. In yet other embodiments, R¹⁰ is triazolyl. In yet other embodiments, R¹⁰ is triazolylmethoxy. In yet other embodiments, R¹⁰ is (N-methyltriazolyl)methyl. In yet other embodiments, R¹⁰ is triazolylmethylamino. In yet other embodiments, R¹⁰ is (N-methyltriazolyl)methylamino. In yet other embodiments, R¹⁰ is CN. In yet other embodiments, R¹⁰ is hydroxymethyl. In yet other embodiments, R¹⁰ is carboxy. In yet other embodiments, R¹⁰ is aminocarbonyl. In yet other embodiments, R¹⁰ is methylaminocarbonyl.

In yet other embodiments, R¹⁰ is dimethylaminocarbonyl. In yet other embodiments, R¹⁰ is methylsulfonyl. In yet other embodiments, R¹⁰ is pyridylmethoxy.

In certain embodiments, the compound of the disclosure is any compound disclosed herein, or a salt, solvate, prodrug, isotopically labelled, stereoisomer, any mixture of stereoisomers, tautomer, and/or any mixture of tautomers thereof.

In certain embodiments, the compound is at least one selected from Table 3, or a salt, solvate, prodrug, isotopically labelled, stereoisomer, any mixture of stereoisomers, tautomer, and/or any mixture of tautomers thereof.

In certain embodiments, the compound is at least one of:
3-(3-chloro-4-fluorophenyl)-1-isobutyl-1-(2-oxo-4-(trifluoromethyl)-1,2,5,6,7,8-hexahydroquinolin-5-yl)urea;
3-(3-chloro-4-fluorophenyl)-1-methyl-1-(2-oxo-4-(trifluoromethyl)-1,2,5,6,7,8-hexahydroquinolin-5-yl)urea;
3-(3-chloro-4-fluorophenyl)-1-(3-hydroxypropyl)-1-(2-oxo-4-(trifluoromethyl)-1,2,5,6,7,8-hexahydroquinolin-5-yl)urea;
3-(3-chloro-4-fluorophenyl)-1-isobutyl-1-(2-oxo-4-(trifluoromethyl)-2,5,6,7-tetrahydro-1H-cyclopenta[b]pyridin-5-yl)urea;
3-(3,4-difluorophenyl)-1-isobutyl-1-(2-oxo-4-(trifluoromethyl)-2,5,6,7-tetrahydro-1H-cyclopenta[b]pyridin-5-yl)urea;
3-(3-chloro-4-fluorophenyl)-1-methyl-1-(6-oxo-1,2,3,4,5,6-hexahydrophenanthridin-1-yl)urea;
3-(3-chloro-4-fluorophenyl)-1-(3-hydroxypropyl)-1-(6-oxo-1,2,3,4,5,6-hexahydrophenanthridin-1-yl)urea;
3-(3-chloro-4-fluorophenyl)-1-isobutyl-1-(6-oxo-1,2,3,4,5,6-hexahydrophenanthridin-1-yl)urea;
3-(3-chloro-4-fluorophenyl)-1-(8-fluoro-6-oxo-1,2,3,4,5,6-hexahydrophenanthridin-1-yl)-1-methylurea;
3-(3-chloro-4-fluorophenyl)-1-(8-fluoro-6-oxo-1,2,3,4,5,6-hexahydrophenanthridin-1-yl)-1-isobutylurea;
3-(3-chloro-4-fluorophenyl)-1-(8-fluoro-6-oxo-1,2,3,4,5,6-hexahydrophenanthridin-1-yl)-1-(3-hydroxypropyl)urea;
3-(3-chloro-4-fluorophenyl)-1-methyl-1-(5-oxo-2,3,4,5-tetrahydro-1H-cyclopenta[c]isoquinolin-1-yl)urea;
3-(3-chloro-4-fluorophenyl)-1-isobutyl-1-(5-oxo-2,3,4,5-tetrahydro-1H-cyclopenta[c]isoquinolin-1-yl)urea;
3-(3-chloro-4-fluorophenyl)-1-(3-hydroxypropyl)-1-(5-oxo-2,3,4,5-tetrahydro-1H-cyclopenta[c]isoquinolin-1-yl)urea;
3-(3-chloro-4-fluorophenyl)-1-(8,9-difluoro-6-oxo-1,2,3,4,5,6-hexahydrophenanthridin-1-yl)-1-methylurea;
3-(3-chloro-4-fluorophenyl)-1-methyl-1-(6-oxo-1,2,3,4,5,6-hexahydrophenanthridin-1-yl)urea;
3-(3-chloro-4-fluorophenyl)-1-methyl-1-(6-oxo-1,4,5,6-tetrahydro-2H-pyrano[3,4-c]isoquinolin-1-yl)urea;
1-(8,9-difluoro-6-oxo-1,2,3,4,5,6-hexahydrophenanthridin-1-yl)-3-(4-fluorophenyl)-1-methylurea;
3-(3-chloro-4-fluorophenyl)-1-(8,10-difluoro-6-oxo-1,2,3,4,5,6-hexahydrophenanthridin-1-yl)-1-methylurea;
3-(3-chloro-4-fluorophenyl)-1-methyl-1-(5-oxo-6,7,8,9,10,11-hexahydro-5H-cyclohepta[c]isoquinolin-11-yl)urea;
3-(3-chloro-4-fluorophenyl)-1-(3-hydroxypropyl)-1-(5-oxo-6,7,8,9,10,11-hexahydro-5H-cyclohepta[c]isoquinolin-11-yl)urea;
3-(3-chloro-4-fluorophenyl)-1-(8-fluoro-6-oxo-1,4,5,6-tetrahydro-2H-pyrano[3,4-c]isoquinolin-1-yl)-1-methylurea;
3-(3-chloro-4-fluorophenyl)-1-(8,9-difluoro-6-oxo-1,4,5,6-tetrahydro-2H-pyrano[3,4-c]isoquinolin-1-yl)-1-methylurea;
3-(3-chloro-4-fluorophenyl)-1-(8,10-difluoro-6-oxo-1,4,5,6-tetrahydro-2H-pyrano[3,4-c]isoquinolin-1-yl)-1-methylurea;
3-(3-chloro-4-fluorophenyl)-1-methyl-1-(3-methyl-6-oxo-1,2,3,4,5,6-hexahydrophenanthridin-1-yl)urea;
3-(3-chloro-4-fluorophenyl)-1-(3,3-dimethyl-6-oxo-1,2,3,4,5,6-hexahydrophenanthridin-1-yl)-1-methylurea;
3-(3-chloro-4-fluorophenyl)-1-(7,8-difluoro-6-oxo-1,2,3,4,5,6-hexahydrophenanthridin-1-yl)-1-methylurea;
3-(3-chloro-5-fluorophenyl)-1-methyl-1-(6-oxo-1,4,5,6-tetrahydro-2H-pyrano[3,4-c]isoquinolin-1-yl)urea;
3-(3-chloro-4-fluorophenyl)-1-isobutyl-1-(6-oxo-1,4,5,6-tetrahydro-2H-pyrano[3,4-c]isoquinolin-1-yl)urea;
3-(3-chloro-4-fluorophenyl)-1-methyl-1-(5-methyl-6-oxo-1,2,3,4,5,6-hexahydrophenanthridin-1-yl)urea;
3-(3,4-difluorophenyl)-1-(8-fluoro-6-oxo-1,2,3,4,5,6-hexahydrophenanthridin-1-yl)-1-methylurea;
1-(8-fluoro-6-oxo-1,2,3,4,5,6-hexahydrophenanthridin-1-yl)-1-methyl-3-(3,4,5-trifluorophenyl)urea;
3-(3-chloro-4-fluorophenyl)-1-(6-methoxy-1,2,3,4-tetrahydrophenanthridin-1-yl)-1-methylurea;
3-(3-chloro-4-fluorophenyl)-1-(7,8-difluoro-6-oxo-1,4,5,6-tetrahydro-2H-pyrano[3,4-c]isoquinolin-1-yl)-1-methylurea;
3-(3-chloro-4-fluorophenyl)-1-(8-fluoro-6-oxo-1,4,5,6-tetrahydro-2H-pyrano[3,4-c]isoquinolin-1-yl)-1-isobutylurea;
3-(3-chloro-4-fluorophenyl)-1-ethyl-1-(8-fluoro-6-oxo-1,4,5,6-tetrahydro-2H-pyrano[3,4-c]isoquinolin-1-yl)urea;
3-(3-chloro-4-fluorophenyl)-1-(8,9-difluoro-6-oxo-1,4,5,6-tetrahydro-2H-pyrano[3,4-c]isoquinolin-1-yl)-1-ethylurea;
3-(3-chloro-4-fluorophenyl)-1-methyl-1-(6-oxo-1,2,3,4,5,6,7,8,9,10-decahydrophenanthridin-1-yl)urea;
3-(3,4-difluorophenyl)-1-methyl-1-(6-oxo-1,2,3,4,5,6,7,8,9,10-decahydrophenanthridin-1-yl)urea;
3-(3,4-difluorophenyl)-1-ethyl-1-(6-oxo-1,2,3,4,5,6,7,8,9,10-decahydrophenanthridin-1-yl)urea;
3-(3-chloro-4-fluorophenyl)-1-ethyl-1-(6-oxo-1,2,3,4,5,6,7,8,9,10-decahydrophenanthridin-1-yl)urea;
3-(3-chloro-4-fluorophenyl)-1-(8-chloro-6-oxo-1,4,5,6-tetrahydro-2H-pyrano[3,4-c]isoquinolin-1-yl)-1-methylurea;
3-(3-chloro-4-fluorophenyl)-1-(8-chloro-6-oxo-1,4,5,6-tetrahydro-2H-pyrano[3,4-c]isoquinolin-1-yl)-1-ethylurea;
3-(4-fluoro-3-methylphenyl)-1-(8-fluoro-6-oxo-1,4,5,6-tetrahydro-2H-pyrano[3,4-c]isoquinolin-1-yl)-1-methylurea;
1-(8-chloro-6-oxo-1,4,5,6-tetrahydro-2H-pyrano[3,4-c]isoquinolin-1-yl)-3-(4-fluoro-3-methylphenyl)-1-methylurea;
1-(8-chloro-6-oxo-1,4,5,6-tetrahydro-2H-pyrano[3,4-c]isoquinolin-1-yl)-1-ethyl-3-(4-fluoro-3-methylphenyl)urea;
3-(3-chloro-4-fluorophenyl)-1-methyl-1-(5-oxo-1,4,5,6,7,8,9,10-octahydro-2H-pyrano[3,4-c]quinolin-10-yl)urea;
3-(3,4-difluorophenyl)-1-methyl-1-(5-oxo-1,4,5,6,7,8,9,10-octahydro-2H-pyrano[3,4-c]quinolin-10-yl)urea;
3-(3-cyano-4-fluorophenyl)-1-(8-fluoro-6-oxo-1,4,5,6-tetrahydro-2H-pyrano[3,4-c]isoquinolin-1-yl)-1-methylurea;
1-(8-chloro-6-oxo-1,4,5,6-tetrahydro-2H-pyrano[3,4-c]isoquinolin-1-yl)-3-(3-cyano-4-fluorophenyl)-1-methylurea;
1-(8-chloro-6-oxo-1,4,5,6-tetrahydro-2H-pyrano[3,4-c]isoquinolin-1-yl)-3-(3-cyano-4-fluorophenyl)-1-ethylurea;
1-(3-chloro-4-fluorophenyl)-3-(8-fluoro-6-oxo-1,4,5,6-tetrahydro-2H-pyrano[3,4-c]isoquinolin-1-yl)urea;
1-(3-chloro-4-fluorophenyl)-3-(8,9-difluoro-6-oxo-1,4,5,6-tetrahydro-2H-pyrano[3,4-c]isoquinolin-1-yl)urea;
1-(8,9-difluoro-6-oxo-1,4,5,6-tetrahydro-2H-pyrano[3,4-c]isoquinolin-1-yl)-3-(4-fluoro-3-methylphenyl)-1-methylurea;
3-(3-chloro-4-fluorophenyl)-1-(9-fluoro-6-oxo-1,4,5,6-tetrahydro-2H-pyrano[3,4-c]isoquinolin-1-yl)-1-methylurea;
3-(3-chloro-4-fluorophenyl)-1-ethyl-1-(9-fluoro-6-oxo-1,4,5,6-tetrahydro-2H-pyrano[3,4-c]isoquinolin-1-yl)urea;
3-(4-fluoro-3-methylphenyl)-1-(9-fluoro-6-oxo-1,4,5,6-tetrahydro-2H-pyrano[3,4-c]isoquinolin-1-yl)-1-methylurea;
1-ethyl-3-(4-fluoro-3-methylphenyl)-1-(9-fluoro-6-oxo-1,4,5,6-tetrahydro-2H-pyrano[3,4-c]isoquinolin-1-yl)urea;
3-(3-cyano-4-fluorophenyl)-1-(9-fluoro-6-oxo-1,4,5,6-tetrahydro-2H-pyrano[3,4-c]isoquinolin-1-yl)-1-methylurea;
3-(3-cyano-4-fluorophenyl)-1-ethyl-1-(9-fluoro-6-oxo-1,4,5,6-tetrahydro-2H-pyrano[3,4-c]isoquinolin-1-yl)urea;
3-(3-chloro-4-fluorophenyl)-1-methyl-1-(5-oxo-3,4,5,6,7,8,9,10-octahydro-1H-pyrano[4,3-c]quinolin-10-yl)urea;
3-(3,4-difluorophenyl)-1-methyl-1-(5-oxo-3,4,5,6,7,8,9,10-octahydro-1H-pyrano[4,3-c]quinolin-10-yl)urea;
3-(3-chloro-4-fluorophenyl)-1-methyl-1-(5-oxo-4,5,6,7,9,10-hexahydro-1H,3H-dipyrano[3,4-b:3′,4′-d]pyridin-10-yl)urea;
3-(3,4-difluorophenyl)-1-methyl-1-(5-oxo-4,5,6,7,9,10-hexahydro-1H,3H-dipyrano[3,4-b:3′,4′-d]pyridin-10-yl)urea;
3-(3-chloro-4-fluorophenyl)-1-(8-cyano-6-oxo-1,4,5,6-tetrahydro-2H-pyrano[3,4-c]isoquinolin-1-yl)-1-methylurea;
3-(3-chloro-4-fluorophenyl)-1-(8-cyano-6-oxo-1,4,5,6-tetrahydro-2H-pyrano[3,4-c]isoquinolin-1-yl)-1-ethylurea;
3-(3-chloro-4-fluorophenyl)-1-methyl-1-(6-oxo-1,2,4,5,6,7,9,10-octahydrodipyrano[3,4-b:4′,3′-d]pyridin-1-yl)urea;
3-(3,4-difluorophenyl)-1-methyl-1-(6-oxo-1,2,4,5,6,7,9,10-octahydrodipyrano[3,4-b:4′,3′-d]pyridin-1-yl)urea;
3-(3-chloro-4-fluorophenyl)-1-methyl-1-(6-oxo-1,4,5,6-tetrahydro-2H-pyrano[3,4-b]thieno[3,2-d]pyridin-1-yl)urea;
3-(3-chloro-4-fluorophenyl)-1-methyl-1-(4-oxo-4,6,8,9-tetrahydro-5H-pyrano[3,4-b]thieno[2,3-d]pyridin-9-yl)urea;
3-(3,5-dichloro-4-fluorophenyl)-1-(8-fluoro-6-oxo-1,4,5,6-tetrahydro-2H-pyrano[3,4-c]isoquinolin-1-yl)-1-methylurea;
3-(3,4-difluorophenyl)-1-(8-fluoro-6-oxo-1,4,5,6-tetrahydro-2H-pyrano[3,4-c]isoquinolin-1-yl)-1-isobutylurea;
1-(8,9-difluoro-6-oxo-1,4,5,6-tetrahydro-2H-pyrano[3,4-c]isoquinolin-1-yl)-1-methyl-3-phenylurea;
1-(8,9-difluoro-6-oxo-1,4,5,6-tetrahydro-2H-pyrano[3,4-c]isoquinolin-1-yl)-3-(4-fluorophenyl)-1-methylurea;
3-(3-chloro-4-fluorophenyl)-1-methyl-1-(4-oxo-4,5,8,9-tetrahydro-6H-pyrano[3,4-b]thieno[3,4-d]pyridin-9-yl)urea;
3-(3-cyano-4-fluorophenyl)-1-(8,9-difluoro-6-oxo-1,4,5,6-tetrahydro-2H-pyrano[3,4-c]isoquinolin-1-yl)-1-methylurea;
3-(3-chloro-4,5-difluorophenyl)-1-(8-fluoro-6-oxo-1,4,5,6-tetrahydro-2H-pyrano[3,4-c]isoquinolin-1-yl)-1-methylurea;
3-(3-chloro-4-fluorophenyl)-1-(8-fluoro-6-oxo-1,4,5,6-tetrahydro-2H-pyrano[3,4-c]isoquinolin-1-yl)-1-(3-hydroxypropyl)urea;
3-(3-chloro-4-fluorophenyl)-1-(8-fluoro-6-oxo-1,4,5,6-tetrahydro-2H-pyrano[3,4-c]isoquinolin-1-yl)-1-(2-hydroxy-2-methylpropyl)urea;
1-(8-fluoro-6-oxo-1,4,5,6-tetrahydro-2H-pyrano[3,4-c]isoquinolin-1-yl)-1-isobutyl-3-(3,4,5-trifluorophenyl)urea;
3-(3-cyano-4-fluorophenyl)-1-(8-fluoro-6-oxo-1,4,5,6-tetrahydro-2H-pyrano[3,4-c]isoquinolin-1-yl)-1-isobutylurea;
1-(8,9-difluoro-6-oxo-1,4,5,6-tetrahydro-2H-pyrano[3,4-c]isoquinolin-1-yl)-3-(3,4-difluorophenyl)-1-methylurea;
3-(3-chlorophenyl)-1-(8,9-difluoro-6-oxo-1,4,5,6-tetrahydro-2H-pyrano[3,4-c]isoquinolin-1-yl)-1-methylurea;
3-(3-cyano-4-fluorophenyl)-1-(8,9-difluoro-6-oxo-1,4,5,6-tetrahydro-2H-pyrano[3,4-c]isoquinolin-1-yl)-1-isobutylurea;
3-(3-(difluoromethyl)-4-fluorophenyl)-1-(8-fluoro-6-oxo-1,4,5,6-tetrahydro-2H-pyrano[3,4-c]isoquinolin-1-yl)-1-methylurea;
1-(8,9-difluoro-6-oxo-1,4,5,6-tetrahydro-2H-pyrano[3,4-c]isoquinolin-1-yl)-3-(3-(difluoromethyl)-4-fluorophenyl)-1-methylurea;
3-(4-fluoro-3-methylphenyl)-1-(8-fluoro-6-oxo-1,4,5,6-tetrahydro-2H-pyrano[3,4-c]isoquinolin-1-yl)-1-isobutylurea;
1-(8,9-difluoro-6-oxo-1,4,5,6-tetrahydro-2H-pyrano[3,4-c]isoquinolin-1-yl)-1-methyl-3-(3,4,5-trifluorophenyl)urea;
1-(3-cyano-4-fluorophenyl)-3-(8,9-difluoro-6-oxo-1,4,5,6-tetrahydro-2H-pyrano[3,4-c]isoquinolin-1-yl)urea;
2-(3-(3-chloro-4-fluorophenyl)-1-(8,9-difluoro-6-oxo-1,4,5,6-tetrahydro-2H-pyrano[3,4-c]isoquinolin-1-yl)ureido)ethane-1-sulfonamide;
3-(3-cyano-4-fluorophenyl)-1-(8,9-difluoro-6-oxo-1,4,5,6-tetrahydro-2H-pyrano[3,4-c]isoquinolin-1-yl)-1-ethylurea;
3-(3-chloro-4-fluorophenyl)-1-(8,9-difluoro-6-oxo-1,4,5,6-tetrahydro-2H-pyrano[3,4-c]isoquinolin-1-yl)-1-(2-(methylsulfonyl)ethyl)urea;
3-(4-chloro-3-fluorophenyl)-1-(8,9-difluoro-6-oxo-1,4,5,6-tetrahydro-2H-pyrano[3,4-c]isoquinolin-1-yl)-1-methylurea;
3-(4-chloro-3-cyanophenyl)-1-(8,9-difluoro-6-oxo-1,4,5,6-tetrahydro-2H-pyrano[3,4-c]isoquinolin-1-yl)-1-methylurea;
3-(3,4-dichlorophenyl)-1-(8,9-difluoro-6-oxo-1,4,5,6-tetrahydro-2H-pyrano[3,4-c]isoquinolin-1-yl)-1-methylurea;
1-(8,9-difluoro-6-oxo-1,2,3,4,5,6-hexahydrophenanthridin-1-yl)-1-methyl-3-(1-(trifluoromethyl)cyclopropyl)urea;
1-(8,9-difluoro-6-oxo-1,4,5,6-tetrahydro-2H-pyrano[3,4-c]isoquinolin-1-yl)-1-methyl-3-(1-(trifluoromethyl)cyclopropyl)urea;
3-(3-Chloro-4-fluorophenyl)-1-(8,9-difluoro-6-oxo-1,4,5,6-tetrahydro-2H-pyrano[3,4-c]isoquinolin-1-yl-1-d)-1-(methyl-d3)urea;
3-(3-Chloro-4-methoxyphenyl)-1-(8,9-difluoro-6-oxo-1,4,5,6-tetrahydro-2H-pyrano[3,4-c]isoquinolin-1-yl)-1-methylurea;
3-(3-Chloro-4-hydroxyphenyl)-1-(8,9-difluoro-6-oxo-1,4,5,6-tetrahydro-2H-pyrano[3,4-c]isoquinolin-1-yl)-1-methylurea;
3-(3-Chloro-4-fluorophenyl)-1-(8,9-difluoro-6-oxo-1,2,3,4,5,6-hexahydrobenzo[c][1,7]naphthyridin-1-yl)-1-methylurea;
3-(3-Chloro-4-fluorophenyl)-1-(8,9-difluoro-4-hydroxy-6-oxo-1,4,5,6-tetrahydro-2H-pyrano[3,4-c]isoquinolin-1-yl)-1-methylurea;
3-(3-chloro-4-Fluorophenyl)-1-(8,9-difluoro-4,6-dioxo-1,4,5,6-tetrahydro-2H-pyrano[3,4-c]isoquinolin-1-yl)-1-methylurea;
3-(3-Chloro-4-fluorophenyl)-1-(8-fluoro-6-oxo-1,2,3,4,5,6-hexahydrobenzo[c][1,7]naphthyridin-1-yl)-1-methylurea;
3-(3-Chloro-4-fluorophenyl)-1-(8-fluoro-3-methyl-6-oxo-1,2,3,4,5,6-hexahydrobenzo[c][1,7]naphthyridin-1-yl)-1-methylurea;
1-(3-Acetyl-8-fluoro-6-oxo-1,2,3,4,5,6-hexahydrobenzo[c][1,7]naphthyridin-1-yl)-3-(3-chloro-4-fluorophenyl)-1-methylurea;
1-(3-Acetyl-8,9-difluoro-6-oxo-1,2,3,4,5,6-hexahydrobenzo[c][1,7]naphthyridin-1-yl)-3-(3-chloro-4-fluorophenyl)-1-methylurea;
3-(3-Cyano-4-fluorophenyl)-1-(8-fluoro-6-oxo-1,2,3,4,5,6-hexahydrobenzo[c][1,7]naphthyridin-1-yl)-1-methylurea;
3-(3-Cyano-4-fluorophenyl)-1-(8-fluoro-3-methyl-6-oxo-1,2,3,4,5,6-hexahydrobenzo[c][1,7]naphthyridin-1-yl)-1-methylurea;
1-(3-Acetyl-8-fluoro-6-oxo-1,2,3,4,5,6-hexahydrobenzo[c][1,7]naphthyridin-1-yl)-3-(3-cyano-4-fluorophenyl)-1-methylurea;
3-(3-Chloro-4-fluorophenyl)-1-(8,9-difluoro-3-methyl-6-oxo-1,2,3,4,5,6-hexahydrobenzo[c][1,7]naphthyridin-1-yl)-1-methylurea;
3-(3-Chloro-4-fluorophenyl)-1-(8-fluoro-3-(2-hydroxyethyl)-6-oxo-1,2,3,4,5,6-hexahydrobenzo[c][1,7]naphthyridin-1-yl)-1-methylurea;
3-(3-Cyano-4-fluorophenyl)-1-(8-fluoro-3-(2-hydroxyethyl)-6-oxo-1,2,3,4,5,6-hexahydrobenzo[c][1,7]naphthyridin-1-yl)-1-methylurea;
3-(3-Cyano-4-fluorophenyl)-1-(8,9-difluoro-3-methyl-6-oxo-1,2,3,4,5,6-hexahydrobenzo[c][1,7]naphthyridin-1-yl)-1-methylurea;
3-(3-Cyano-4-fluorophenyl)-1-(8,9-difluoro-6-oxo-1,2,3,4,5,6-hexahydrobenzo[c][1,7]naphthyridin-1-yl)-1-methylurea;
1-(3-Acetyl-8,9-difluoro-6-oxo-1,2,3,4,5,6-hexahydrobenzo[c][1,7]naphthyridin-1-yl)-3-(3-cyano-4-fluorophenyl)-1-methylurea;
3-(3-Chloro-4-fluorophenyl)-1-(8,9-difluoro-3-(2-hydroxyethyl)-6-oxo-1,2,3,4,5,6-hexahydrobenzo[c][1,7]naphthyridin-1-yl)-1-methylurea;
3-(3-Cyano-4-fluorophenyl)-1-(8,9-difluoro-3-(2-hydroxyethyl)-6-oxo-1,2,3,4,5,6-hexahydrobenzo[c][1,7]naphthyridine-1-yl)-1-methylurea;
3-(3-Chloro-4-fluorophenyl)-1-(8-fluoro-6-oxo-1,4,5,6-tetrahydro-2H-thiopyrano[3,4-c]isoquinolin-1-yl)-1-methylurea;
3-(3-Cyano-4-fluorophenyl)-1-(8-fluoro-6-oxo-1,4,5,6-tetrahydro-2H-thiopyrano[3,4-c]isoquinolin-1-yl)-1-methylurea;
3-(3-Chloro-4-fluorophenyl)-1-(8,9-difluoro-6-oxo-1,4,5,6-tetrahydro-2H-thiopyrano[3,4-c]isoquinolin-1-yl)-1-methylurea;
3-(3-Cyano-4-fluorophenyl)-1-(8,9-difluoro-6-oxo-1,4,5,6-tetrahydro-2H-thiopyrano[3,4-c]isoquinolin-1-yl)-1-methylurea;
1-(8,9-Difluoro-6-oxo-1,2,3,4,5,6-hexahydrobenzo[c][1,7]naphthyridin-1-yl)-3-(3,4-difluorophenyl)-1-methylurea;
3-(3-Chloro-4-fluorophenyl)-1-(8-fluoro-3-oxido-6-oxo-1,4,5,6-tetrahydro-2H-thiopyrano[3,4-c]isoquinolin-1-yl)-1-methylurea;
3-(3-Chloro-4-fluorophenyl)-1-(8-fluoro-3,3-dioxido-6-oxo-1,4,5,6-tetrahydro-2H-thiopyrano[3,4-c]isoquinolin-1-yl)-1-methylurea;
3-(3-Cyano-4-fluorophenyl)-1-(8-fluoro-3-oxido-6-oxo-1,4,5,6-tetrahydro-2H-thiopyrano[3,4-c]isoquinolin-1-yl)-1-methylurea;
3-(3-Chloro-4-fluorophenyl)-1-(8,9-difluoro-3,3-dioxido-6-oxo-1,4,5,6-tetrahydro-2H-thiopyrano[3,4-c]isoquinolin-1-yl)-1-methylurea;
3-(3-Cyano-4-fluorophenyl)-1-(8,9-difluoro-3,3-dioxido-6-oxo-1,4,5,6-tetrahydro-2H-thiopyrano[3,4-c]isoquinolin-1-yl)-1-methylurea;
3-(3-Chloro-4-fluorophenyl)-1-(8,9-difluoro-3-oxido-6-oxo-1,4,5,6-tetrahydro-2H-thiopyrano[3,4-c]isoquinolin-1-yl)-1-methylurea;
3-(3-Cyano-4-fluorophenyl)-1-(8,9-difluoro-3-oxido-6-oxo-1,4,5,6-tetrahydro-2H-thiopyrano[3,4-c]isoquinolin-1-yl)-1-methylurea;
3-(3-Cyano-4-fluorophenyl)-1-(8-fluoro-3,3-dioxido-6-oxo-1,4,5,6-tetrahydro-2H-thiopyrano[3,4-c]isoquinolin-1-yl)-1-methylurea;
1-(8,9-difluoro-6-oxo-1,2,3,4,5,6-hexahydrobenzo[c][1,7]naphthyridin-1-yl)-3-(3-(difluoromethyl)-4-fluorophenyl)-1-methylurea;
N-(8,9-difluoro-6-oxo-1,4,5,6-tetrahydro-2H-pyrano[3,4-c]isoquinolin-1-yl)-N-methylisoindoline-2-carboxamide;
5-chloro-N-(8,9-difluoro-6-oxo-1,4,5,6-tetrahydro-2H-pyrano[3,4-c]isoquinolin-1-yl)-N-methylisoindoline-2-carboxamide;
5-bromo-N-(8,9-difluoro-6-oxo-1,4,5,6-tetrahydro-2H-pyrano[3,4-c]isoquinolin-1-yl)-N-methylisoindoline-2-carboxamide;
5-fluoro-N-(8,9-difluoro-6-oxo-1,4,5,6-tetrahydro-2H-pyrano[3,4-c]isoquinolin-1-yl)-N-methylisoindoline-2-carboxamide;
N-(8,9-difluoro-6-oxo-1,2,3,4,5,6-hexahydrobenzo[c][1,7]naphthyridin-1-yl)-N-methylisoindoline-2-carboxamide;
N-(8,9-difluoro-6-oxo-1,2,3,4,5,6-hexahydrobenzo[c][1,7]naphthyridin-1-yl)-5-fluoro-N-methylisoindoline-2-carboxamide;
N-(8,9-difluoro-6-oxo-1,2,3,4,5,6-hexahydrobenzo[c][1,7]naphthyridin-1-yl)-5-chloro-N-methylisoindoline-2-carboxamide;
N-(8,9-difluoro-6-oxo-1,2,3,4,5,6-hexahydrobenzo[c][1,7]naphthyridin-1-yl)-5-bromo-N-methylisoindoline-2-carboxamide;
N-(8,9-difluoro-6-oxo-1,4,5,6-tetrahydro-2H-pyrano[3,4-c]isoquinolin-1-yl)-N-methyl-5-(trifluoromethyl)isoindoline-2-carboxamide;
N-(8,9-difluoro-6-oxo-1,2,3,4,5,6-hexahydrobenzo[c][1,7]naphthyridin-1-yl)-N-methyl-5-(trifluoromethyl)isoindoline-2-carboxamide;
1-(8,9-difluoro-5-methyl-6-oxo-1,4,5,6-tetrahydro-2H-pyrano[3,4-c]isoquinolin-1-yl)-3-(3-(difluoromethyl)-4-fluorophenyl)-1-methylurea;
1-(8,9-difluoro-6-methoxy-1,4-dihydro-2H-pyrano[3,4-c]isoquinolin-1-yl)-3-(3-(difluoromethyl)-4-fluorophenyl)-1-methylurea;
1-(8,9-difluoro-6-(methylamino)-1,4-dihydro-2H-pyrano[3,4-c]isoquinolin-1-yl)-3-(3-(difluoromethyl)-4-fluorophenyl)-1-methylurea
1-(8,9-difluoro-6-((2-hydroxyethyl)amino)-1,4-dihydro-2H-pyrano[3,4-c]isoquinolin-1-yl)-3-(3-(difluoromethyl)-4-fluorophenyl)-1-methylurea;
1-(8,9-difluoro-6-((2-aminoethyl)amino)-1,4-dihydro-2H-pyrano[3,4-c]isoquinolin-1-yl)-3-(3-(difluoromethyl)-4-fluorophenyl)-1-methylurea
3-(3-chloro-4-fluorophenyl)-1-(8,9-difluoro-6-((2-aminoethyl)amino)-1,4-dihydro-2H-pyrano[3,4-c]isoquinolin-1-yl)-1-methylurea;
3-(3-chloro-4-fluorophenyl)-1-(8,9-difluoro-6-(methylamino)-1,4-dihydro-2H-pyrano[3,4-c]isoquinolin-1-yl)-1-methylurea;
1-(8,9-difluoro-5-methyl-6-oxo-1,4,5,6-tetrahydro-2H-pyrano[3,4-c]isoquinolin-1-yl)-3-(3-chloro-4-fluorophenyl)-1-methylurea;
3-(3-chloro-4-fluorophenyl)-1-(8,9-difluoro-6-methoxy-1,4-dihydro-2H-pyrano[3,4-c]isoquinolin-1-yl)-1-methylurea;
3-(3-chloro-4-fluorophenyl)-1-(8,9-difluoro-6-((2-hydroxyethyl)amino)-1,4-dihydro-2H-pyrano[3,4-c]isoquinolin-1-yl)-1-methylurea;
1-(8,9-difluoro-5-(2-hydroxyethyl)-6-oxo-1,4,5,6-tetrahydro-2H-pyrano[3,4-c]isoquinolin-1-yl)-3-(3-(difluoromethyl)-4-fluorophenyl)-1-methylurea;
1-(6-(2-aminoethoxy)-8,9-difluoro-1,4-dihydro-2H-pyrano[3,4-c]isoquinolin-1-yl)-3-(3-(difluoromethyl)-4-fluorophenyl)-1-methylurea;
1-(5-(2-aminoethyl)-8,9-difluoro-6-oxo-1,4,5,6-tetrahydro-2H-pyrano[3,4-c]isoquinolin-1-yl)-3-(3-(difluoromethyl)-4-fluorophenyl)-1-methylurea;
3-(3-chloro-4-fluorophenyl)-1-(8,9-difluoro-5-(2-hydroxyethyl)-6-oxo-1,4,5,6-tetrahydro-2H-pyrano[3,4-c]isoquinolin-1-yl)-1-methylurea;
3-(3-chloro-4-fluorophenyl)-1-(8,9-difluoro-6-(2-hydroxyethoxy)-1,4-dihydro-2H-pyrano[3,4-c]isoquinolin-1-yl)-1-methylurea;
1-(8,9-difluoro-6-(2-hydroxyethoxy)-1,4-dihydro-2H-pyrano[3,4-c]isoquinolin-1-yl)-3-(3-(difluoromethyl)-4-fluorophenyl)-1-methylurea;
1-(5-(2-aminoethyl)-8,9-difluoro-6-oxo-1,4,5,6-tetrahydro-2H-pyrano[3,4-c]isoquinolin-1-yl)-3-(3-chloro-4-fluorophenyl)-1-methylurea;
1-(6-(2-aminoethoxy)-8,9-difluoro-1,4-dihydro-2H-pyrano[3,4-c]isoquinolin-1-yl)-3-(3-chloro-4-fluorophenyl)-1-methylurea;
or a salt, solvate, prodrug, isotopically labelled, stereoisomer, any mixture of stereoisomers,
tautomer, and/or any mixture of tautomers thereof.
In certain embodiments, the compound is at least one of:
(R)-3-(3-chloro-4-fluorophenyl)-1-isobutyl-1-(2-oxo-4-(trifluoromethyl)-1,2,5,6,7,8-hexahydroquinolin-5-yl)urea;
(S)-3-(3-chloro-4-fluorophenyl)-1-isobutyl-1-(2-oxo-4-(trifluoromethyl)-1,2,5,6,7,8-hexahydroquinolin-5-yl)urea;
(R)-3-(3-chloro-4-fluorophenyl)-1-methyl-1-(2-oxo-4-(trifluoromethyl)-1,2,5,6,7,8-hexahydroquinolin-5-yl)urea;
(S)-3-(3-chloro-4-fluorophenyl)-1-methyl-1-(2-oxo-4-(trifluoromethyl)-1,2,5,6,7,8-hexahydroquinolin-5-yl)urea;
(R)-3-(3-chloro-4-fluorophenyl)-1-(3-hydroxypropyl)-1-(2-oxo-4-(trifluoromethyl)-1,2,5,6,7,8-hexahydroquinolin-5-yl)urea;
(S)-3-(3-chloro-4-fluorophenyl)-1-(3-hydroxypropyl)-1-(2-oxo-4-(trifluoromethyl)-1,2,5,6,7,8-hexahydroquinolin-5-yl)urea;
(R)-3-(3-chloro-4-fluorophenyl)-1-isobutyl-1-(2-oxo-4-(trifluoromethyl)-2,5,6,7-tetrahydro-1H-cyclopenta[b]pyridin-5-yl)urea;
(S)-3-(3-chloro-4-fluorophenyl)-1-isobutyl-1-(2-oxo-4-(trifluoromethyl)-2,5,6,7-tetrahydro-1H-cyclopenta[b]pyridin-5-yl)urea;
(R)-3-(3,4-difluorophenyl)-1-isobutyl-1-(2-oxo-4-(trifluoromethyl)-2,5,6,7-tetrahydro-1H-cyclopenta[b]pyridin-5-yl)urea;
(S)-3-(3,4-difluorophenyl)-1-isobutyl-1-(2-oxo-4-(trifluoromethyl)-2,5,6,7-tetrahydro-1H-cyclopenta[b]pyridin-5-yl)urea;
(R)-3-(3-chloro-4-fluorophenyl)-1-methyl-1-(6-oxo-1,2,3,4,5,6-hexahydrophenanthridin-1-yl)urea;
(S)-3-(3-chloro-4-fluorophenyl)-1-methyl-1-(6-oxo-1,2,3,4,5,6-hexahydrophenanthridin-1-yl)urea;
(R)-3-(3-chloro-4-fluorophenyl)-1-(3-hydroxypropyl)-1-(6-oxo-1,2,3,4,5,6-hexahydrophenanthridin-1-yl)urea;
(S)-3-(3-chloro-4-fluorophenyl)-1-(3-hydroxypropyl)-1-(6-oxo-1,2,3,4,5,6-hexahydrophenanthridin-1-yl)urea;
(R)-3-(3-chloro-4-fluorophenyl)-1-isobutyl-1-(6-oxo-1,2,3,4,5,6-hexahydrophenanthridin-1-yl)urea;
(S)-3-(3-chloro-4-fluorophenyl)-1-isobutyl-1-(6-oxo-1,2,3,4,5,6-hexahydrophenanthridin-1-yl)urea;
(R)-3-(3-chloro-4-fluorophenyl)-1-(8-fluoro-6-oxo-1,2,3,4,5,6-hexahydrophenanthridin-1-yl)-1-methylurea;
(S)-3-(3-chloro-4-fluorophenyl)-1-(8-fluoro-6-oxo-1,2,3,4,5,6-hexahydrophenanthridin-1-yl)-1-methylurea;
(R)-3-(3-chloro-4-fluorophenyl)-1-(8-fluoro-6-oxo-1,2,3,4,5,6-hexahydrophenanthridin-1-yl)-1-isobutylurea;
(S)-3-(3-chloro-4-fluorophenyl)-1-(8-fluoro-6-oxo-1,2,3,4,5,6-hexahydrophenanthridin-1-yl)-1-isobutylurea;
(R)-3-(3-chloro-4-fluorophenyl)-1-(8-fluoro-6-oxo-1,2,3,4,5,6-hexahydrophenanthridin-1-yl)-1-(3-hydroxypropyl)urea;
(S)-3-(3-chloro-4-fluorophenyl)-1-(8-fluoro-6-oxo-1,2,3,4,5,6-hexahydrophenanthridin-1-yl)-1-(3-hydroxypropyl)urea;
(R)-3-(3-chloro-4-fluorophenyl)-1-methyl-1-(5-oxo-2,3,4,5-tetrahydro-1H-cyclopenta[c]isoquinolin-1-yl)urea;
(S)-3-(3-chloro-4-fluorophenyl)-1-methyl-1-(5-oxo-2,3,4,5-tetrahydro-1H-cyclopenta[c]isoquinolin-1-yl)urea;
(R)-3-(3-chloro-4-fluorophenyl)-1-isobutyl-1-(5-oxo-2,3,4,5-tetrahydro-1H-cyclopenta[c]isoquinolin-1-yl)urea;
(S)-3-(3-chloro-4-fluorophenyl)-1-isobutyl-1-(5-oxo-2,3,4,5-tetrahydro-1H-cyclopenta[c]isoquinolin-1-yl)urea;
(R)-3-(3-chloro-4-fluorophenyl)-1-(3-hydroxypropyl)-1-(5-oxo-2,3,4,5-tetrahydro-1H-cyclopenta[c]isoquinolin-1-yl)urea;
(S)-3-(3-chloro-4-fluorophenyl)-1-(3-hydroxypropyl)-1-(5-oxo-2,3,4,5-tetrahydro-1H-cyclopenta[c]isoquinolin-1-yl)urea;
(R)-3-(3-chloro-4-fluorophenyl)-1-(8,9-difluoro-6-oxo-1,2,3,4,5,6-hexahydrophenanthridin-1-yl)-1-methylurea;
(S)-3-(3-chloro-4-fluorophenyl)-1-(8,9-difluoro-6-oxo-1,2,3,4,5,6-hexahydrophenanthridin-1-yl)-1-methylurea;
(R)-3-(3-chloro-4-fluorophenyl)-1-methyl-1-(6-oxo-1,2,3,4,5,6-hexahydrophenanthridin-1-yl)urea;
(S)-3-(3-chloro-4-fluorophenyl)-1-methyl-1-(6-oxo-1,2,3,4,5,6-hexahydrophenanthridin-1-yl)urea;
(R)-3-(3-chloro-4-fluorophenyl)-1-methyl-1-(6-oxo-1,4,5,6-tetrahydro-2H-pyrano[3,4-c]isoquinolin-1-yl)urea;
(S)-3-(3-chloro-4-fluorophenyl)-1-methyl-1-(6-oxo-1,4,5,6-tetrahydro-2H-pyrano[3,4-c]isoquinolin-1-yl)urea;
(R)-1-(8,9-difluoro-6-oxo-1,2,3,4,5,6-hexahydrophenanthridin-1-yl)-3-(4-fluorophenyl)-1-methylurea;
(S)-1-(8,9-difluoro-6-oxo-1,2,3,4,5,6-hexahydrophenanthridin-1-yl)-3-(4-fluorophenyl)-1-methylurea;
(R)-3-(3-chloro-4-fluorophenyl)-1-(8,10-difluoro-6-oxo-1,2,3,4,5,6-hexahydrophenanthridin-1-yl)-1-methylurea;
(S)-3-(3-chloro-4-fluorophenyl)-1-(8,10-difluoro-6-oxo-1,2,3,4,5,6-hexahydrophenanthridin-1-yl)-1-methylurea;
(R)-3-(3-chloro-4-fluorophenyl)-1-methyl-1-(5-oxo-6,7,8,9,10,11-hexahydro-5H-cyclohepta[c]isoquinolin-11-yl)urea;
(S)-3-(3-chloro-4-fluorophenyl)-1-methyl-1-(5-oxo-6,7,8,9,10,11-hexahydro-5H-cyclohepta[c]isoquinolin-11-yl)urea;
(R)-3-(3-chloro-4-fluorophenyl)-1-(3-hydroxypropyl)-1-(5-oxo-6,7,8,9,10,11-hexahydro-5H-cyclohepta[c]isoquinolin-11-yl)urea;
(S)-3-(3-chloro-4-fluorophenyl)-1-(3-hydroxypropyl)-1-(5-oxo-6,7,8,9,10,11-hexahydro-5H-cyclohepta[c]isoquinolin-11-yl)urea;
(R)-3-(3-chloro-4-fluorophenyl)-1-(8-fluoro-6-oxo-1,4,5,6-tetrahydro-2H-pyrano[3,4-c]isoquinolin-1-yl)-1-methylurea;
(S)-3-(3-chloro-4-fluorophenyl)-1-(8-fluoro-6-oxo-1,4,5,6-tetrahydro-2H-pyrano[3,4-c]isoquinolin-1-yl)-1-methylurea;
(R)-3-(3-chloro-4-fluorophenyl)-1-(8,9-difluoro-6-oxo-1,4,5,6-tetrahydro-2H-pyrano[3,4-c]isoquinolin-1-yl)-1-methylurea;
(S)-3-(3-chloro-4-fluorophenyl)-1-(8,9-difluoro-6-oxo-1,4,5,6-tetrahydro-2H-pyrano[3,4-c]isoquinolin-1-yl)-1-methylurea;
(R)-3-(3-chloro-4-fluorophenyl)-1-(8,10-difluoro-6-oxo-1,4,5,6-tetrahydro-2H-pyrano[3,4-c]isoquinolin-1-yl)-1-methylurea;
(S)-3-(3-chloro-4-fluorophenyl)-1-(8,10-difluoro-6-oxo-1,4,5,6-tetrahydro-2H-pyrano[3,4-c]isoquinolin-1-yl)-1-methylurea;
3-(3-chloro-4-fluorophenyl)-1-methyl-(1R)-(3R-methyl-6-oxo-1,2,3,4,5,6-hexahydrophenanthridin-1-yl)urea;
3-(3-chloro-4-fluorophenyl)-1-methyl-(1R)-(3 S-methyl-6-oxo-1,2,3,4,5,6-hexahydrophenanthridin-1-yl)urea;
3-(3-chloro-4-fluorophenyl)-1-methyl-(1S)-(3R-methyl-6-oxo-1,2,3,4,5,6-hexahydrophenanthridin-1-yl)urea;
3-(3-chloro-4-fluorophenyl)-1-methyl-(1S)-(3S-methyl-6-oxo-1,2,3,4,5,6-hexahydrophenanthridin-1-yl)urea;
(R)-3-(3-chloro-4-fluorophenyl)-1-(3,3-dimethyl-6-oxo-1,2,3,4,5,6-hexahydrophenanthridin-1-yl)-1-methylurea;
(S)-3-(3-chloro-4-fluorophenyl)-1-(3,3-dimethyl-6-oxo-1,2,3,4,5,6-hexahydrophenanthridin-1-yl)-1-methylurea;
(R)-3-(3-chloro-4-fluorophenyl)-1-(7,8-difluoro-6-oxo-1,2,3,4,5,6-hexahydrophenanthridin-1-yl)-1-methylurea;
(S)-3-(3-chloro-4-fluorophenyl)-1-(7,8-difluoro-6-oxo-1,2,3,4,5,6-hexahydrophenanthridin-1-yl)-1-methylurea;
(R)-3-(3-chloro-5-fluorophenyl)-1-methyl-1-(6-oxo-1,4,5,6-tetrahydro-2H-pyrano[3,4-c]isoquinolin-1-yl)urea;
(S)-3-(3-chloro-5-fluorophenyl)-1-methyl-1-(6-oxo-1,4,5,6-tetrahydro-2H-pyrano[3,4-c]isoquinolin-1-yl)urea;
(R)-3-(3-chloro-4-fluorophenyl)-1-isobutyl-1-(6-oxo-1,4,5,6-tetrahydro-2H-pyrano[3,4-c]isoquinolin-1-yl)urea;
(S)-3-(3-chloro-4-fluorophenyl)-1-isobutyl-1-(6-oxo-1,4,5,6-tetrahydro-2H-pyrano[3,4-c]isoquinolin-1-yl)urea;
(R)-3-(3-chloro-4-fluorophenyl)-1-methyl-1-(5-methyl-6-oxo-1,2,3,4,5,6-hexahydrophenanthridin-1-yl)urea;
(S)-3-(3-chloro-4-fluorophenyl)-1-methyl-1-(5-methyl-6-oxo-1,2,3,4,5,6-hexahydrophenanthridin-1-yl)urea;
(R)-3-(3,4-difluorophenyl)-1-(8-fluoro-6-oxo-1,2,3,4,5,6-hexahydrophenanthridin-1-yl)-1-methylurea;
(S)-3-(3,4-difluorophenyl)-1-(8-fluoro-6-oxo-1,2,3,4,5,6-hexahydrophenanthridin-1-yl)-1-methylurea;
(R)-1-(8-fluoro-6-oxo-1,2,3,4,5,6-hexahydrophenanthridin-1-yl)-1-methyl-3-(3,4,5-trifluorophenyl)urea;
(S)-1-(8-fluoro-6-oxo-1,2,3,4,5,6-hexahydrophenanthridin-1-yl)-1-methyl-3-(3,4,5-trifluorophenyl)urea;
(R)-3-(3-chloro-4-fluorophenyl)-1-(6-methoxy-1,2,3,4-tetrahydrophenanthridin-1-yl)-1-methylurea;
(S)-3-(3-chloro-4-fluorophenyl)-1-(6-methoxy-1,2,3,4-tetrahydrophenanthridin-1-yl)-1-methylurea;
(R)-3-(3-chloro-4-fluorophenyl)-1-(7,8-difluoro-6-oxo-1,4,5,6-tetrahydro-2H-pyrano[3,4-c]isoquinolin-1-yl)-1-methylurea;
(S)-3-(3-chloro-4-fluorophenyl)-1-(7,8-difluoro-6-oxo-1,4,5,6-tetrahydro-2H-pyrano[3,4-c]isoquinolin-1-yl)-1-methylurea;
(R)-3-(3-chloro-4-fluorophenyl)-1-(8-fluoro-6-oxo-1,4,5,6-tetrahydro-2H-pyrano[3,4-c]isoquinolin-1-yl)-1-isobutylurea;
(S)-3-(3-chloro-4-fluorophenyl)-1-(8-fluoro-6-oxo-1,4,5,6-tetrahydro-2H-pyrano[3,4-c]isoquinolin-1-yl)-1-isobutylurea;
(R)-3-(3-chloro-4-fluorophenyl)-1-ethyl-1-(8-fluoro-6-oxo-1,4,5,6-tetrahydro-2H-pyrano[3,4-c]isoquinolin-1-yl)urea;
(S)-3-(3-chloro-4-fluorophenyl)-1-ethyl-1-(8-fluoro-6-oxo-1,4,5,6-tetrahydro-2H-pyrano[3,4-c]isoquinolin-1-yl)urea;
(R)-3-(3-chloro-4-fluorophenyl)-1-(8,9-difluoro-6-oxo-1,4,5,6-tetrahydro-2H-pyrano[3,4-c]isoquinolin-1-yl)-1-ethylurea;
(S)-3-(3-chloro-4-fluorophenyl)-1-(8,9-difluoro-6-oxo-1,4,5,6-tetrahydro-2H-pyrano[3,4-c]isoquinolin-1-yl)-1-ethylurea;
(R)-3-(3-chloro-4-fluorophenyl)-1-methyl-1-(6-oxo-1,2,3,4,5,6,7,8,9,10-decahydrophenanthridin-1-yl)urea;
(S)-3-(3-chloro-4-fluorophenyl)-1-methyl-1-(6-oxo-1,2,3,4,5,6,7,8,9,10-decahydrophenanthridin-1-yl)urea;
(R)-3-(3,4-difluorophenyl)-1-methyl-1-(6-oxo-1,2,3,4,5,6,7,8,9,10-decahydrophenanthridin-1-yl)urea;
(S)-3-(3,4-difluorophenyl)-1-methyl-1-(6-oxo-1,2,3,4,5,6,7,8,9,10-decahydrophenanthridin-1-yl)urea;
(R)-3-(3,4-difluorophenyl)-1-ethyl-1-(6-oxo-1,2,3,4,5,6,7,8,9,10-decahydrophenanthridin-1-yl)urea;
(S)-3-(3,4-difluorophenyl)-1-ethyl-1-(6-oxo-1,2,3,4,5,6,7,8,9,10-decahydrophenanthridin-1-yl)urea;
(R)-3-(3-chloro-4-fluorophenyl)-1-ethyl-1-(6-oxo-1,2,3,4,5,6,7,8,9,10-decahydrophenanthridin-1-yl)urea;
(S)-3-(3-chloro-4-fluorophenyl)-1-ethyl-1-(6-oxo-1,2,3,4,5,6,7,8,9,10-decahydrophenanthridin-1-yl)urea;
(R)-3-(3-chloro-4-fluorophenyl)-1-(8-chloro-6-oxo-1,4,5,6-tetrahydro-2H-pyrano[3,4-c]isoquinolin-1-yl)-1-methylurea;
(S)-3-(3-chloro-4-fluorophenyl)-1-(8-chloro-6-oxo-1,4,5,6-tetrahydro-2H-pyrano[3,4-c]isoquinolin-1-yl)-1-methylurea;
(R)-3-(3-chloro-4-fluorophenyl)-1-(8-chloro-6-oxo-1,4,5,6-tetrahydro-2H-pyrano[3,4-c]isoquinolin-1-yl)-1-ethylurea;
(S)-3-(3-chloro-4-fluorophenyl)-1-(8-chloro-6-oxo-1,4,5,6-tetrahydro-2H-pyrano[3,4-c]isoquinolin-1-yl)-1-ethylurea;
(R)-3-(4-fluoro-3-methylphenyl)-1-(8-fluoro-6-oxo-1,4,5,6-tetrahydro-2H-pyrano[3,4-c]isoquinolin-1-yl)-1-methylurea;
(S)-3-(4-fluoro-3-methylphenyl)-1-(8-fluoro-6-oxo-1,4,5,6-tetrahydro-2H-pyrano[3,4-c]isoquinolin-1-yl)-1-methylurea;
(R)-1-(8-chloro-6-oxo-1,4,5,6-tetrahydro-2H-pyrano[3,4-c]isoquinolin-1-yl)-3-(4-fluoro-3-methylphenyl)-1-methylurea;
(S)-1-(8-chloro-6-oxo-1,4,5,6-tetrahydro-2H-pyrano[3,4-c]isoquinolin-1-yl)-3-(4-fluoro-3-methylphenyl)-1-methylurea;
(R)-1-(8-chloro-6-oxo-1,4,5,6-tetrahydro-2H-pyrano[3,4-c]isoquinolin-1-yl)-1-ethyl-3-(4-fluoro-3-methylphenyl)urea;
(S)-1-(8-chloro-6-oxo-1,4,5,6-tetrahydro-2H-pyrano[3,4-c]isoquinolin-1-yl)-1-ethyl-3-(4-fluoro-3-methylphenyl)urea;
(R)-3-(3-chloro-4-fluorophenyl)-1-methyl-1-(5-oxo-1,4,5,6,7,8,9,10-octahydro-2H-pyrano[3,4-c]quinolin-10-yl)urea;
(S)-3-(3-chloro-4-fluorophenyl)-1-methyl-1-(5-oxo-1,4,5,6,7,8,9,10-octahydro-2H-pyrano[3,4-c]quinolin-10-yl)urea;
(R)-3-(3,4-difluorophenyl)-1-methyl-1-(5-oxo-1,4,5,6,7,8,9,10-octahydro-2H-pyrano[3,4-c]quinolin-10-yl)urea;
(S)-3-(3,4-difluorophenyl)-1-methyl-1-(5-oxo-1,4,5,6,7,8,9,10-octahydro-2H-pyrano[3,4-c]quinolin-10-yl)urea;
(R)-3-(3-cyano-4-fluorophenyl)-1-(8-fluoro-6-oxo-1,4,5,6-tetrahydro-2H-pyrano[3,4-c]isoquinolin-1-yl)-1-methylurea;
(S)-3-(3-cyano-4-fluorophenyl)-1-(8-fluoro-6-oxo-1,4,5,6-tetrahydro-2H-pyrano[3,4-c]isoquinolin-1-yl)-1-methylurea;
(R)-1-(8-chloro-6-oxo-1,4,5,6-tetrahydro-2H-pyrano[3,4-c]isoquinolin-1-yl)-3-(3-cyano-4-fluorophenyl)-1-methylurea;
(S)-1-(8-chloro-6-oxo-1,4,5,6-tetrahydro-2H-pyrano[3,4-c]isoquinolin-1-yl)-3-(3-cyano-4-fluorophenyl)-1-methylurea;
(R)-1-(8-chloro-6-oxo-1,4,5,6-tetrahydro-2H-pyrano[3,4-c]isoquinolin-1-yl)-3-(3-cyano-4-fluorophenyl)-1-ethylurea;
(S)-1-(8-chloro-6-oxo-1,4,5,6-tetrahydro-2H-pyrano[3,4-c]isoquinolin-1-yl)-3-(3-cyano-4-fluorophenyl)-1-ethylurea;
(R)-1-(3-chloro-4-fluorophenyl)-3-(8-fluoro-6-oxo-1,4,5,6-tetrahydro-2H-pyrano[3,4-c]isoquinolin-1-yl)urea;
(S)-1-(3-chloro-4-fluorophenyl)-3-(8-fluoro-6-oxo-1,4,5,6-tetrahydro-2H-pyrano[3,4-c]isoquinolin-1-yl)urea;
(R)-1-(3-chloro-4-fluorophenyl)-3-(8,9-difluoro-6-oxo-1,4,5,6-tetrahydro-2H-pyrano[3,4-c]isoquinolin-1-yl)urea;
(S)-1-(3-chloro-4-fluorophenyl)-3-(8,9-difluoro-6-oxo-1,4,5,6-tetrahydro-2H-pyrano[3,4-c]isoquinolin-1-yl)urea;
(R)-1-(8,9-difluoro-6-oxo-1,4,5,6-tetrahydro-2H-pyrano[3,4-c]isoquinolin-1-yl)-3-(4-fluoro-3-methylphenyl)-1-methylurea;
(S)-1-(8,9-difluoro-6-oxo-1,4,5,6-tetrahydro-2H-pyrano[3,4-c]isoquinolin-1-yl)-3-(4-fluoro-3-methylphenyl)-1-methylurea;
(R)-3-(3-chloro-4-fluorophenyl)-1-(9-fluoro-6-oxo-1,4,5,6-tetrahydro-2H-pyrano[3,4-c]isoquinolin-1-yl)-1-methylurea;
(S)-3-(3-chloro-4-fluorophenyl)-1-(9-fluoro-6-oxo-1,4,5,6-tetrahydro-2H-pyrano[3,4-c]isoquinolin-1-yl)-1-methylurea;
(R)-3-(3-chloro-4-fluorophenyl)-1-ethyl-1-(9-fluoro-6-oxo-1,4,5,6-tetrahydro-2H-pyrano[3,4-c]isoquinolin-1-yl)urea;
(S)-3-(3-chloro-4-fluorophenyl)-1-ethyl-1-(9-fluoro-6-oxo-1,4,5,6-tetrahydro-2H-pyrano[3,4-c]isoquinolin-1-yl)urea;
(R)-3-(4-fluoro-3-methylphenyl)-1-(9-fluoro-6-oxo-1,4,5,6-tetrahydro-2H-pyrano[3,4-c]isoquinolin-1-yl)-1-methylurea;
(S)-3-(4-fluoro-3-methylphenyl)-1-(9-fluoro-6-oxo-1,4,5,6-tetrahydro-2H-pyrano[3,4-c]isoquinolin-1-yl)-1-methylurea;
(R)-1-ethyl-3-(4-fluoro-3-methylphenyl)-1-(9-fluoro-6-oxo-1,4,5,6-tetrahydro-2H-pyrano[3,4-c]isoquinolin-1-yl)urea;
(S)-1-ethyl-3-(4-fluoro-3-methylphenyl)-1-(9-fluoro-6-oxo-1,4,5,6-tetrahydro-2H-pyrano[3,4-c]isoquinolin-1-yl)urea;
(R)-3-(3-cyano-4-fluorophenyl)-1-(9-fluoro-6-oxo-1,4,5,6-tetrahydro-2H-pyrano[3,4-c]isoquinolin-1-yl)-1-methylurea;
(S)-3-(3-cyano-4-fluorophenyl)-1-(9-fluoro-6-oxo-1,4,5,6-tetrahydro-2H-pyrano[3,4-c]isoquinolin-1-yl)-1-methylurea;
(R)-3-(3-cyano-4-fluorophenyl)-1-ethyl-1-(9-fluoro-6-oxo-1,4,5,6-tetrahydro-2H-pyrano[3,4-c]isoquinolin-1-yl)urea;
(S)-3-(3-cyano-4-fluorophenyl)-1-ethyl-1-(9-fluoro-6-oxo-1,4,5,6-tetrahydro-2H-pyrano[3,4-c]isoquinolin-1-yl)urea;
(R)-3-(3-chloro-4-fluorophenyl)-1-methyl-1-(5-oxo-3,4,5,6,7,8,9,10-octahydro-1H-pyrano[4,3-c]quinolin-10-yl)urea;
(S)-3-(3-chloro-4-fluorophenyl)-1-methyl-1-(5-oxo-3,4,5,6,7,8,9,10-octahydro-1H-pyrano[4,3-c]quinolin-10-yl)urea;
(R)-3-(3,4-difluorophenyl)-1-methyl-1-(5-oxo-3,4,5,6,7,8,9,10-octahydro-1H-pyrano[4,3-c]quinolin-10-yl)urea;
(S)-3-(3,4-difluorophenyl)-1-methyl-1-(5-oxo-3,4,5,6,7,8,9,10-octahydro-1H-pyrano[4,3-c]quinolin-10-yl)urea;
(R)-3-(3-chloro-4-fluorophenyl)-1-methyl-1-(5-oxo-4,5,6,7,9,10-hexahydro-1H,3H-dipyrano[3,4-b:3′,4′-d]pyridin-10-yl)urea;
(S)-3-(3-chloro-4-fluorophenyl)-1-methyl-1-(5-oxo-4,5,6,7,9,10-hexahydro-1H,3H-dipyrano[3,4-b:3′,4′-d]pyridin-10-yl)urea;
(R)-3-(3,4-difluorophenyl)-1-methyl-1-(5-oxo-4,5,6,7,9,10-hexahydro-1H,3H-dipyrano[3,4-b:3′,4′-d]pyridin-10-yl)urea;
(S)-3-(3,4-difluorophenyl)-1-methyl-1-(5-oxo-4,5,6,7,9,10-hexahydro-1H,3H-dipyrano[3,4-b:3′,4′-d]pyridin-10-yl)urea;
(R)-3-(3-chloro-4-fluorophenyl)-1-(8-cyano-6-oxo-1,4,5,6-tetrahydro-2H-pyrano[3,4-c]isoquinolin-1-yl)-1-methylurea;
(S)-3-(3-chloro-4-fluorophenyl)-1-(8-cyano-6-oxo-1,4,5,6-tetrahydro-2H-pyrano[3,4-c]isoquinolin-1-yl)-1-methylurea;
(R)-3-(3-chloro-4-fluorophenyl)-1-(8-cyano-6-oxo-1,4,5,6-tetrahydro-2H-pyrano[3,4-c]isoquinolin-1-yl)-1-ethylurea;
(S)-3-(3-chloro-4-fluorophenyl)-1-(8-cyano-6-oxo-1,4,5,6-tetrahydro-2H-pyrano[3,4-c]isoquinolin-1-yl)-1-ethylurea;
(R)-3-(3-chloro-4-fluorophenyl)-1-methyl-1-(6-oxo-1,2,4,5,6,7,9,10-octahydrodipyrano[3,4-b:4′,3′-d]pyridin-1-yl)urea;
(S)-3-(3-chloro-4-fluorophenyl)-1-methyl-1-(6-oxo-1,2,4,5,6,7,9,10-octahydrodipyrano[3,4-b:4′,3′-d]pyridin-1-yl)urea;
(R)-3-(3,4-difluorophenyl)-1-methyl-1-(6-oxo-1,2,4,5,6,7,9,10-octahydrodipyrano[3,4-b:4′,3′-d]pyridin-1-yl)urea;
(S)-3-(3,4-difluorophenyl)-1-methyl-1-(6-oxo-1,2,4,5,6,7,9,10-octahydrodipyrano[3,4-b:4′,3′-d]pyridin-1-yl)urea;
(R)-3-(3-chloro-4-fluorophenyl)-1-methyl-1-(6-oxo-1,4,5,6-tetrahydro-2H-pyrano[3,4-b]thieno[3,2-d]pyridin-1-yl)urea;
(S)-3-(3-chloro-4-fluorophenyl)-1-methyl-1-(6-oxo-1,4,5,6-tetrahydro-2H-pyrano[3,4-b]thieno[3,2-d]pyridin-1-yl)urea;
(R)-3-(3-chloro-4-fluorophenyl)-1-methyl-1-(4-oxo-4,6,8,9-tetrahydro-5H-pyrano[3,4-b]thieno[2,3-d]pyridin-9-yl)urea;
(S)-3-(3-chloro-4-fluorophenyl)-1-methyl-1-(4-oxo-4,6,8,9-tetrahydro-5H-pyrano[3,4-b]thieno[2,3-d]pyridin-9-yl)urea;
(R)-3-(3,5-dichloro-4-fluorophenyl)-1-(8-fluoro-6-oxo-1,4,5,6-tetrahydro-2H-pyrano[3,4-c]isoquinolin-1-yl)-1-methylurea;
(S)-3-(3,5-dichloro-4-fluorophenyl)-1-(8-fluoro-6-oxo-1,4,5,6-tetrahydro-2H-pyrano[3,4-c]isoquinolin-1-yl)-1-methylurea;
(R)-3-(3,4-difluorophenyl)-1-(8-fluoro-6-oxo-1,4,5,6-tetrahydro-2H-pyrano[3,4-c]isoquinolin-1-yl)-1-isobutylurea;
(S)-3-(3,4-difluorophenyl)-1-(8-fluoro-6-oxo-1,4,5,6-tetrahydro-2H-pyrano[3,4-c]isoquinolin-1-yl)-1-isobutylurea;
(R)-1-(8,9-difluoro-6-oxo-1,4,5,6-tetrahydro-2H-pyrano[3,4-c]isoquinolin-1-yl)-1-methyl-3-phenylurea;
(S)-1-(8,9-difluoro-6-oxo-1,4,5,6-tetrahydro-2H-pyrano[3,4-c]isoquinolin-1-yl)-1-methyl-3-phenylurea;
(R)-1-(8,9-difluoro-6-oxo-1,4,5,6-tetrahydro-2H-pyrano[3,4-c]isoquinolin-1-yl)-3-(4-fluorophenyl)-1-methylurea;
(S)-1-(8,9-difluoro-6-oxo-1,4,5,6-tetrahydro-2H-pyrano[3,4-c]isoquinolin-1-yl)-3-(4-fluorophenyl)-1-methylurea;
(R)-3-(3-chloro-4-fluorophenyl)-1-methyl-1-(4-oxo-4,5,8,9-tetrahydro-6H-pyrano[3,4-b]thieno[3,4-d]pyridin-9-yl)urea;
(S)-3-(3-chloro-4-fluorophenyl)-1-methyl-1-(4-oxo-4,5,8,9-tetrahydro-6H-pyrano[3,4-b]thieno[3,4-d]pyridin-9-yl)urea;
(R)-3-(3-cyano-4-fluorophenyl)-1-(8,9-difluoro-6-oxo-1,4,5,6-tetrahydro-2H-pyrano[3,4-c]isoquinolin-1-yl)-1-methylurea;
(S)-3-(3-cyano-4-fluorophenyl)-1-(8,9-difluoro-6-oxo-1,4,5,6-tetrahydro-2H-pyrano[3,4-c]isoquinolin-1-yl)-1-methylurea;
(R)-3-(3-chloro-4,5-difluorophenyl)-1-(8-fluoro-6-oxo-1,4,5,6-tetrahydro-2H-pyrano[3,4-c]isoquinolin-1-yl)-1-methylurea;
(S)-3-(3-chloro-4,5-difluorophenyl)-1-(8-fluoro-6-oxo-1,4,5,6-tetrahydro-2H-pyrano[3,4-c]isoquinolin-1-yl)-1-methylurea;
(R)-3-(3-chloro-4-fluorophenyl)-1-(8-fluoro-6-oxo-1,4,5,6-tetrahydro-2H-pyrano[3,4-c]isoquinolin-1-yl)-1-(3-hydroxypropyl)urea;
(S)-3-(3-chloro-4-fluorophenyl)-1-(8-fluoro-6-oxo-1,4,5,6-tetrahydro-2H-pyrano[3,4-c]isoquinolin-1-yl)-1-(3-hydroxypropyl)urea;
(R)-3-(3-chloro-4-fluorophenyl)-1-(8-fluoro-6-oxo-1,4,5,6-tetrahydro-2H-pyrano[3,4-c]isoquinolin-1-yl)-1-(2-hydroxy-2-methylpropyl)urea;
(S)-3-(3-chloro-4-fluorophenyl)-1-(8-fluoro-6-oxo-1,4,5,6-tetrahydro-2H-pyrano[3,4-c]isoquinolin-1-yl)-1-(2-hydroxy-2-methylpropyl)urea;
(R)-1-(8-fluoro-6-oxo-1,4,5,6-tetrahydro-2H-pyrano[3,4-c]isoquinolin-1-yl)-1-isobutyl-3-(3,4,5-trifluorophenyl)urea;
(S)-1-(8-fluoro-6-oxo-1,4,5,6-tetrahydro-2H-pyrano[3,4-c]isoquinolin-1-yl)-1-isobutyl-3-(3,4,5-trifluorophenyl)urea;
(R)-3-(3-cyano-4-fluorophenyl)-1-(8-fluoro-6-oxo-1,4,5,6-tetrahydro-2H-pyrano[3,4-c]isoquinolin-1-yl)-1-isobutylurea;
(S)-3-(3-cyano-4-fluorophenyl)-1-(8-fluoro-6-oxo-1,4,5,6-tetrahydro-2H-pyrano[3,4-c]isoquinolin-1-yl)-1-isobutylurea;
(R)-1-(8,9-difluoro-6-oxo-1,4,5,6-tetrahydro-2H-pyrano[3,4-c]isoquinolin-1-yl)-3-(3,4-difluorophenyl)-1-methylurea;
(S)-1-(8,9-difluoro-6-oxo-1,4,5,6-tetrahydro-2H-pyrano[3,4-c]isoquinolin-1-yl)-3-(3,4-difluorophenyl)-1-methylurea;
(R)-3-(3-chlorophenyl)-1-(8,9-difluoro-6-oxo-1,4,5,6-tetrahydro-2H-pyrano[3,4-c]isoquinolin-1-yl)-1-methylurea;
(S)-3-(3-chlorophenyl)-1-(8,9-difluoro-6-oxo-1,4,5,6-tetrahydro-2H-pyrano[3,4-c]isoquinolin-1-yl)-1-methylurea;
(R)-3-(3-cyano-4-fluorophenyl)-1-(8,9-difluoro-6-oxo-1,4,5,6-tetrahydro-2H-pyrano[3,4-c]isoquinolin-1-yl)-1-isobutylurea;
(S)-3-(3-cyano-4-fluorophenyl)-1-(8,9-difluoro-6-oxo-1,4,5,6-tetrahydro-2H-pyrano[3,4-c]isoquinolin-1-yl)-1-isobutylurea;
(R)-3-(3-(difluoromethyl)-4-fluorophenyl)-1-(8-fluoro-6-oxo-1,4,5,6-tetrahydro-2H-pyrano[3,4-c]isoquinolin-1-yl)-1-methylurea;
(S)-3-(3-(difluoromethyl)-4-fluorophenyl)-1-(8-fluoro-6-oxo-1,4,5,6-tetrahydro-2H-pyrano[3,4-c]isoquinolin-1-yl)-1-methylurea;
(R)-1-(8,9-difluoro-6-oxo-1,4,5,6-tetrahydro-2H-pyrano[3,4-c]isoquinolin-1-yl)-3-(3-(difluoromethyl)-4-fluorophenyl)-1-methylurea;
(S)-1-(8,9-difluoro-6-oxo-1,4,5,6-tetrahydro-2H-pyrano[3,4-c]isoquinolin-1-yl)-3-(3-(difluoromethyl)-4-fluorophenyl)-1-methylurea;
(R)-3-(4-fluoro-3-methylphenyl)-1-(8-fluoro-6-oxo-1,4,5,6-tetrahydro-2H-pyrano[3,4-c]isoquinolin-1-yl)-1-isobutylurea;
(S)-3-(4-fluoro-3-methylphenyl)-1-(8-fluoro-6-oxo-1,4,5,6-tetrahydro-2H-pyrano[3,4-c]isoquinolin-1-yl)-1-isobutylurea;
(R)-1-(8,9-difluoro-6-oxo-1,4,5,6-tetrahydro-2H-pyrano[3,4-c]isoquinolin-1-yl)-1-methyl-3-(3,4,5-trifluorophenyl)urea;
(S)-1-(8,9-difluoro-6-oxo-1,4,5,6-tetrahydro-2H-pyrano[3,4-c]isoquinolin-1-yl)-1-methyl-3-(3,4,5-trifluorophenyl)urea;
(R)-1-(3-cyano-4-fluorophenyl)-3-(8,9-difluoro-6-oxo-1,4,5,6-tetrahydro-2H-pyrano[3,4-c]isoquinolin-1-yl)urea;
(S)-1-(3-cyano-4-fluorophenyl)-3-(8,9-difluoro-6-oxo-1,4,5,6-tetrahydro-2H-pyrano[3,4-c]isoquinolin-1-yl)urea;
(R)-2-(3-(3-chloro-4-fluorophenyl)-1-(8,9-difluoro-6-oxo-1,4,5,6-tetrahydro-2H-pyrano[3,4-c]isoquinolin-1-yl)ureido)ethane-1-sulfonamide;
(S)-2-(3-(3-chloro-4-fluorophenyl)-1-(8,9-difluoro-6-oxo-1,4,5,6-tetrahydro-2H-pyrano[3,4-c]isoquinolin-1-yl)ureido)ethane-1-sulfonamide;
(R)-3-(3-cyano-4-fluorophenyl)-1-(8,9-difluoro-6-oxo-1,4,5,6-tetrahydro-2H-pyrano[3,4-c]isoquinolin-1-yl)-1-ethylurea;
(S)-3-(3-cyano-4-fluorophenyl)-1-(8,9-difluoro-6-oxo-1,4,5,6-tetrahydro-2H-pyrano[3,4-c]isoquinolin-1-yl)-1-ethylurea;
(R)-3-(3-chloro-4-fluorophenyl)-1-(8,9-difluoro-6-oxo-1,4,5,6-tetrahydro-2H-pyrano[3,4-c]isoquinolin-1-yl)-1-(2-(methylsulfonyl)ethyl)urea;
(S)-3-(3-chloro-4-fluorophenyl)-1-(8,9-difluoro-6-oxo-1,4,5,6-tetrahydro-2H-pyrano[3,4-c]isoquinolin-1-yl)-1-(2-(methylsulfonyl)ethyl)urea;
(R)-3-(4-chloro-3-fluorophenyl)-1-(8,9-difluoro-6-oxo-1,4,5,6-tetrahydro-2H-pyrano[3,4-c]isoquinolin-1-yl)-1-methylurea;
(S)-3-(4-chloro-3-fluorophenyl)-1-(8,9-difluoro-6-oxo-1,4,5,6-tetrahydro-2H-pyrano[3,4-c]isoquinolin-1-yl)-1-methylurea;
(R)-3-(4-chloro-3-cyanophenyl)-1-(8,9-difluoro-6-oxo-1,4,5,6-tetrahydro-2H-pyrano[3,4-c]isoquinolin-1-yl)-1-methylurea;
(S)-3-(4-chloro-3-cyanophenyl)-1-(8,9-difluoro-6-oxo-1,4,5,6-tetrahydro-2H-pyrano[3,4-c]isoquinolin-1-yl)-1-methylurea;
(R)-3-(3,4-dichlorophenyl)-1-(8,9-difluoro-6-oxo-1,4,5,6-tetrahydro-2H-pyrano[3,4-c]isoquinolin-1-yl)-1-methylurea;
(S)-3-(3,4-dichlorophenyl)-1-(8,9-difluoro-6-oxo-1,4,5,6-tetrahydro-2H-pyrano[3,4-c]isoquinolin-1-yl)-1-methylurea;
(R)-1-(8,9-difluoro-6-oxo-1,2,3,4,5,6-hexahydrophenanthridin-1-yl)-1-methyl-3-(1-(trifluoromethyl)cyclopropyl)urea;
(S)-1-(8,9-difluoro-6-oxo-1,2,3,4,5,6-hexahydrophenanthridin-1-yl)-1-methyl-3-(1-(trifluoromethyl)cyclopropyl)urea;
(R)-1-(8,9-difluoro-6-oxo-1,4,5,6-tetrahydro-2H-pyrano[3,4-c]isoquinolin-1-yl)-1-methyl-3-(1-(trifluoromethyl)cyclopropyl)urea;
(S)-1-(8,9-difluoro-6-oxo-1,4,5,6-tetrahydro-2H-pyrano[3,4-c]isoquinolin-1-yl)-1-methyl-3-(1-(trifluoromethyl)cyclopropyl)urea;
(R)-3-(3-Chloro-4-fluorophenyl)-1-(8,9-difluoro-6-oxo-1,4,5,6-tetrahydro-2H-pyrano[3,4-c]isoquinolin-1-yl-1-d)-1-(methyl-d3)urea;
(S)-3-(3-Chloro-4-fluorophenyl)-1-(8,9-difluoro-6-oxo-1,4,5,6-tetrahydro-2H-pyrano[3,4-c]isoquinolin-1-yl-1-d)-1-(methyl-d3)urea;
(R)-3-(3-Chloro-4-methoxyphenyl)-1-(8,9-difluoro-6-oxo-1,4,5,6-tetrahydro-2H-pyrano[3,4-c]isoquinolin-1-yl)-1-methylurea;
(S)-3-(3-Chloro-4-methoxyphenyl)-1-(8,9-difluoro-6-oxo-1,4,5,6-tetrahydro-2H-pyrano[3,4-c]isoquinolin-1-yl)-1-methylurea;
(R)-3-(3-Chloro-4-hydroxyphenyl)-1-(8,9-difluoro-6-oxo-1,4,5,6-tetrahydro-2H-pyrano[3,4-c]isoquinolin-1-yl)-1-methylurea;
(S)-3-(3-Chloro-4-hydroxyphenyl)-1-(8,9-difluoro-6-oxo-1,4,5,6-tetrahydro-2H-pyrano[3,4-c]isoquinolin-1-yl)-1-methylurea;
(R)-3-(3-Chloro-4-fluorophenyl)-1-(8,9-difluoro-6-oxo-1,2,3,4,5,6-hexahydrobenzo[c][1,7]naphthyridin-1-yl)-1-methylurea;
(S)-3-(3-Chloro-4-fluorophenyl)-1-(8,9-difluoro-6-oxo-1,2,3,4,5,6-hexahydrobenzo[c][1,7]naphthyridin-1-yl)-1-methylurea;
3-(3-Chloro-4-fluorophenyl)-(1R)-(8,9-difluoro-4R-hydroxy-6-oxo-1,4,5,6-tetrahydro-2H-pyrano[3,4-c]isoquinolin-1-yl)-1-methylurea;
3-(3-Chloro-4-fluorophenyl)-(1R)-(8,9-difluoro-4S-hydroxy-6-oxo-1,4,5,6-tetrahydro-2H-pyrano[3,4-c]isoquinolin-1-yl)-1-methylurea;
3-(3-Chloro-4-fluorophenyl)-(1S)-(8,9-difluoro-4R-hydroxy-6-oxo-1,4,5,6-tetrahydro-2H-pyrano[3,4-c]isoquinolin-1-yl)-1-methylurea;
3-(3-Chloro-4-fluorophenyl)-(1S)-(8,9-difluoro-4S-hydroxy-6-oxo-1,4,5,6-tetrahydro-2H-pyrano[3,4-c]isoquinolin-1-yl)-1-methylurea;
(R)-3-(3-chloro-4-Fluorophenyl)-1-(8,9-difluoro-4,6-dioxo-1,4,5,6-tetrahydro-2H-pyrano[3,4-c]isoquinolin-1-yl)-1-methylurea;
(S)-3-(3-chloro-4-Fluorophenyl)-1-(8,9-difluoro-4,6-dioxo-1,4,5,6-tetrahydro-2H-pyrano[3,4-c]isoquinolin-1-yl)-1-methylurea;
(R)-3-(3-Chloro-4-fluorophenyl)-1-(8-fluoro-6-oxo-1,2,3,4,5,6-hexahydrobenzo[c][1,7]naphthyridin-1-yl)-1-methylurea;
(S)-3-(3-Chloro-4-fluorophenyl)-1-(8-fluoro-6-oxo-1,2,3,4,5,6-hexahydrobenzo[c][1,7]naphthyridin-1-yl)-1-methylurea;
(R)-3-(3-Chloro-4-fluorophenyl)-1-(8-fluoro-3-methyl-6-oxo-1,2,3,4,5,6-hexahydrobenzo[c][1,7]naphthyridin-1-yl)-1-methylurea;
(S)-3-(3-Chloro-4-fluorophenyl)-1-(8-fluoro-3-methyl-6-oxo-1,2,3,4,5,6-hexahydrobenzo[c][1,7]naphthyridin-1-yl)-1-methylurea;
(R)-1-(3-Acetyl-8-fluoro-6-oxo-1,2,3,4,5,6-hexahydrobenzo[c][1,7]naphthyridin-1-yl)-3-(3-chloro-4-fluorophenyl)-1-methylurea;
(S)-1-(3-Acetyl-8-fluoro-6-oxo-1,2,3,4,5,6-hexahydrobenzo[c][1,7]naphthyridin-1-yl)-3-(3-chloro-4-fluorophenyl)-1-methylurea;
(R)-1-(3-Acetyl-8,9-difluoro-6-oxo-1,2,3,4,5,6-hexahydrobenzo[c][1,7]naphthyridin-1-yl)-3-(3-chloro-4-fluorophenyl)-1-methylurea;
(S)-1-(3-Acetyl-8,9-difluoro-6-oxo-1,2,3,4,5,6-hexahydrobenzo[c][1,7]naphthyridin-1-yl)-3-(3-chloro-4-fluorophenyl)-1-methylurea;
(R)-3-(3-Cyano-4-fluorophenyl)-1-(8-fluoro-6-oxo-1,2,3,4,5,6-hexahydrobenzo[c][1,7]naphthyridin-1-yl)-1-methylurea;
(S)-3-(3-Cyano-4-fluorophenyl)-1-(8-fluoro-6-oxo-1,2,3,4,5,6-hexahydrobenzo[c][1,7]naphthyridin-1-yl)-1-methylurea;
(R)-3-(3-Cyano-4-fluorophenyl)-1-(8-fluoro-3-methyl-6-oxo-1,2,3,4,5,6-hexahydrobenzo[c][1,7]naphthyridin-1-yl)-1-methylurea;
(S)-3-(3-Cyano-4-fluorophenyl)-1-(8-fluoro-3-methyl-6-oxo-1,2,3,4,5,6-hexahydrobenzo[c][1,7]naphthyridin-1-yl)-1-methylurea;
(R)-1-(3-Acetyl-8-fluoro-6-oxo-1,2,3,4,5,6-hexahydrobenzo[c][1,7]naphthyridin-1-yl)-3-(3-cyano-4-fluorophenyl)-1-methylurea;
(S)-1-(3-Acetyl-8-fluoro-6-oxo-1,2,3,4,5,6-hexahydrobenzo[c][1,7]naphthyridin-1-yl)-3-(3-cyano-4-fluorophenyl)-1-methylurea;
(R)-3-(3-Chloro-4-fluorophenyl)-1-(8,9-difluoro-3-methyl-6-oxo-1,2,3,4,5,6-hexahydrobenzo[c][1,7]naphthyridin-1-yl)-1-methylurea;
(S)-3-(3-Chloro-4-fluorophenyl)-1-(8,9-difluoro-3-methyl-6-oxo-1,2,3,4,5,6-hexahydrobenzo[c][1,7]naphthyridin-1-yl)-1-methylurea;
(R)-3-(3-Chloro-4-fluorophenyl)-1-(8-fluoro-3-(2-hydroxyethyl)-6-oxo-1,2,3,4,5,6-hexahydrobenzo[c][1,7]naphthyridin-1-yl)-1-methylurea;
(S)-3-(3-Chloro-4-fluorophenyl)-1-(8-fluoro-3-(2-hydroxyethyl)-6-oxo-1,2,3,4,5,6-hexahydrobenzo[c][1,7]naphthyridin-1-yl)-1-methylurea;
(R)-3-(3-Cyano-4-fluorophenyl)-1-(8-fluoro-3-(2-hydroxyethyl)-6-oxo-1,2,3,4,5,6-hexahydrobenzo[c][1,7]naphthyridin-1-yl)-1-methylurea;
(S)-3-(3-Cyano-4-fluorophenyl)-1-(8-fluoro-3-(2-hydroxyethyl)-6-oxo-1,2,3,4,5,6-hexahydrobenzo[c][1,7]naphthyridin-1-yl)-1-methylurea;
(R)-3-(3-Cyano-4-fluorophenyl)-1-(8,9-difluoro-3-methyl-6-oxo-1,2,3,4,5,6-hexahydrobenzo[c][1,7]naphthyridin-1-yl)-1-methylurea;
(S)-3-(3-Cyano-4-fluorophenyl)-1-(8,9-difluoro-3-methyl-6-oxo-1,2,3,4,5,6-hexahydrobenzo[c][1,7]naphthyridin-1-yl)-1-methylurea;
(R)-3-(3-Cyano-4-fluorophenyl)-1-(8,9-difluoro-6-oxo-1,2,3,4,5,6-hexahydrobenzo[c][1,7]naphthyridin-1-yl)-1-methylurea;
(S)-3-(3-Cyano-4-fluorophenyl)-1-(8,9-difluoro-6-oxo-1,2,3,4,5,6-hexahydrobenzo[c][1,7]naphthyridin-1-yl)-1-methylurea;
(R)-1-(3-Acetyl-8,9-difluoro-6-oxo-1,2,3,4,5,6-hexahydrobenzo[c][1,7]naphthyridin-1-yl)-3-(3-cyano-4-fluorophenyl)-1-methylurea;
(S)-1-(3-Acetyl-8,9-difluoro-6-oxo-1,2,3,4,5,6-hexahydrobenzo[c][1,7]naphthyridin-1-yl)-3-(3-cyano-4-fluorophenyl)-1-methylurea;
(R)-3-(3-Chloro-4-fluorophenyl)-1-(8,9-difluoro-3-(2-hydroxyethyl)-6-oxo-1,2,3,4,5,6-hexahydrobenzo[c][1,7]naphthyridin-1-yl)-1-methylurea;
(S)-3-(3-Chloro-4-fluorophenyl)-1-(8,9-difluoro-3-(2-hydroxyethyl)-6-oxo-1,2,3,4,5,6-hexahydrobenzo[c][1,7]naphthyridin-1-yl)-1-methylurea;
(R)-3-(3-Cyano-4-fluorophenyl)-1-(8,9-difluoro-3-(2-hydroxyethyl)-6-oxo-1,2,3,4,5,6-hexahydrobenzo[c][1,7]naphthyridine-1-yl)-1-methylurea;
(S)-3-(3-Cyano-4-fluorophenyl)-1-(8,9-difluoro-3-(2-hydroxyethyl)-6-oxo-1,2,3,4,5,6-hexahydrobenzo[c][1,7]naphthyridine-1-yl)-1-methylurea;
(R)-3-(3-Chloro-4-fluorophenyl)-1-(8-fluoro-6-oxo-1,4,5,6-tetrahydro-2H-thiopyrano[3,4-c]isoquinolin-1-yl)-1-methylurea;
(S)-3-(3-Chloro-4-fluorophenyl)-1-(8-fluoro-6-oxo-1,4,5,6-tetrahydro-2H-thiopyrano[3,4-c]isoquinolin-1-yl)-1-methylurea;
(R)-3-(3-Cyano-4-fluorophenyl)-1-(8-fluoro-6-oxo-1,4,5,6-tetrahydro-2H-thiopyrano[3,4-c]isoquinolin-1-yl)-1-methylurea;
(S)-3-(3-Cyano-4-fluorophenyl)-1-(8-fluoro-6-oxo-1,4,5,6-tetrahydro-2H-thiopyrano[3,4-c]isoquinolin-1-yl)-1-methylurea;
(R)-3-(3-Chloro-4-fluorophenyl)-1-(8,9-difluoro-6-oxo-1,4,5,6-tetrahydro-2H-thiopyrano[3,4-c]isoquinolin-1-yl)-1-methylurea;
(S)-3-(3-Chloro-4-fluorophenyl)-1-(8,9-difluoro-6-oxo-1,4,5,6-tetrahydro-2H-thiopyrano[3,4-c]isoquinolin-1-yl)-1-methylurea;
(R)-3-(3-Cyano-4-fluorophenyl)-1-(8,9-difluoro-6-oxo-1,4,5,6-tetrahydro-2H-thiopyrano[3,4-c]isoquinolin-1-yl)-1-methylurea;
(S)-3-(3-Cyano-4-fluorophenyl)-1-(8,9-difluoro-6-oxo-1,4,5,6-tetrahydro-2H-thiopyrano[3,4-c]isoquinolin-1-yl)-1-methylurea;
(R)-1-(8,9-Difluoro-6-oxo-1,2,3,4,5,6-hexahydrobenzo[c][1,7]naphthyridin-1-yl)-3-(3,4-difluorophenyl)-1-methylurea;
(S)-1-(8,9-Difluoro-6-oxo-1,2,3,4,5,6-hexahydrobenzo[c][1,7]naphthyridin-1-yl)-3-(3,4-difluorophenyl)-1-methylurea;
3-(3-Chloro-4-fluorophenyl)-(1R)-(8-fluoro-3R-oxido-6-oxo-1,4,5,6-tetrahydro-2H-thiopyrano[3,4-c]isoquinolin-1-yl)-1-methylurea;
3-(3-Chloro-4-fluorophenyl)-(1R)-(8-fluoro-3 S-oxido-6-oxo-1,4,5,6-tetrahydro-2H-thiopyrano[3,4-c]isoquinolin-1-yl)-1-methylurea;
3-(3-Chloro-4-fluorophenyl)-(1S)-(8-fluoro-3R-oxido-6-oxo-1,4,5,6-tetrahydro-2H-thiopyrano[3,4-c]isoquinolin-1-yl)-1-methylurea;
3-(3-Chloro-4-fluorophenyl)-(1S)-(8-fluoro-3S-oxido-6-oxo-1,4,5,6-tetrahydro-2H-thiopyrano[3,4-c]isoquinolin-1-yl)-1-methylurea;
(R)-3-(3-Chloro-4-fluorophenyl)-1-(8-fluoro-3,3-dioxido-6-oxo-1,4,5,6-tetrahydro-2H-thiopyrano[3,4-c]isoquinolin-1-yl)-1-methylurea;
(S)-3-(3-Chloro-4-fluorophenyl)-1-(8-fluoro-3,3-dioxido-6-oxo-1,4,5,6-tetrahydro-2H-thiopyrano[3,4-c]isoquinolin-1-yl)-1-methylurea;
3-(3-Cyano-4-fluorophenyl)-(1R)-(8-fluoro-3R-oxido-6-oxo-1,4,5,6-tetrahydro-2H-thiopyrano[3,4-c]isoquinolin-1-yl)-1-methylurea;
3-(3-Cyano-4-fluorophenyl)-(1R)-(8-fluoro-3 S-oxido-6-oxo-1,4,5,6-tetrahydro-2H-thiopyrano[3,4-c]isoquinolin-1-yl)-1-methylurea;
3-(3-Cyano-4-fluorophenyl)-(1S)-(8-fluoro-3R-oxido-6-oxo-1,4,5,6-tetrahydro-2H-thiopyrano[3,4-c]isoquinolin-1-yl)-1-methylurea;
3-(3-Cyano-4-fluorophenyl)-(1S)-(8-fluoro-3S-oxido-6-oxo-1,4,5,6-tetrahydro-2H-thiopyrano[3,4-c]isoquinolin-1-yl)-1-methylurea;
(R)-3-(3-Chloro-4-fluorophenyl)-1-(8,9-difluoro-3,3-dioxido-6-oxo-1,4,5,6-tetrahydro-2H-thiopyrano[3,4-c]isoquinolin-1-yl)-1-methylurea;
(S)-3-(3-Chloro-4-fluorophenyl)-1-(8,9-difluoro-3,3-dioxido-6-oxo-1,4,5,6-tetrahydro-2H-thiopyrano[3,4-c]isoquinolin-1-yl)-1-methylurea;
(R)-3-(3-Cyano-4-fluorophenyl)-1-(8,9-difluoro-3,3-dioxido-6-oxo-1,4,5,6-tetrahydro-2H-thiopyrano[3,4-c]isoquinolin-1-yl)-1-methylurea;
(S)-3-(3-Cyano-4-fluorophenyl)-1-(8,9-difluoro-3,3-dioxido-6-oxo-1,4,5,6-tetrahydro-2H-thiopyrano[3,4-c]isoquinolin-1-yl)-1-methylurea;
3-(3-Chloro-4-fluorophenyl)-(1R)-(8,9-difluoro-3R-oxido-6-oxo-1,4,5,6-tetrahydro-2H-thiopyrano[3,4-c]isoquinolin-1-yl)-1-methylurea;
3-(3-Chloro-4-fluorophenyl)-(1R)-(8,9-difluoro-3S-oxido-6-oxo-1,4,5,6-tetrahydro-2H-thiopyrano[3,4-c]isoquinolin-1-yl)-1-methylurea;
3-(3-Chloro-4-fluorophenyl)-(1S)-(8,9-difluoro-3R-oxido-6-oxo-1,4,5,6-tetrahydro-2H-thiopyrano[3,4-c]isoquinolin-1-yl)-1-methylurea;
3-(3-Chloro-4-fluorophenyl)-(1S)-(8,9-difluoro-3S-oxido-6-oxo-1,4,5,6-tetrahydro-2H-thiopyrano[3,4-c]isoquinolin-1-yl)-1-methylurea;
3-(3-Cyano-4-fluorophenyl)-(1R)-(8,9-difluoro-3R-oxido-6-oxo-1,4,5,6-tetrahydro-2H-thiopyrano[3,4-c]isoquinolin-1-yl)-1-methylurea;
3-(3-Cyano-4-fluorophenyl)-(1R)-(8,9-difluoro-3 S-oxido-6-oxo-1,4,5,6-tetrahydro-2H-thiopyrano[3,4-c]isoquinolin-1-yl)-1-methylurea;
3-(3-Cyano-4-fluorophenyl)-(1S)-(8,9-difluoro-3R-oxido-6-oxo-1,4,5,6-tetrahydro-2H-thiopyrano[3,4-c]isoquinolin-1-yl)-1-methylurea;
3-(3-Cyano-4-fluorophenyl)-(1S)-(8,9-difluoro-3S-oxido-6-oxo-1,4,5,6-tetrahydro-2H-thiopyrano[3,4-c]isoquinolin-1-yl)-1-methylurea;
(R)-3-(3-Cyano-4-fluorophenyl)-1-(8-fluoro-3,3-dioxido-6-oxo-1,4,5,6-tetrahydro-2H-thiopyrano[3,4-c]isoquinolin-1-yl)-1-methylurea;
(S)-3-(3-Cyano-4-fluorophenyl)-1-(8-fluoro-3,3-dioxido-6-oxo-1,4,5,6-tetrahydro-2H-thiopyrano[3,4-c]isoquinolin-1-yl)-1-methylurea;
(R)-1-(8,9-difluoro-6-oxo-1,2,3,4,5,6-hexahydrobenzo[c][1,7]naphthyridin-1-yl)-3-(3-(difluoromethyl)-4-fluorophenyl)-1-methylurea;
(S)-1-(8,9-difluoro-6-oxo-1,2,3,4,5,6-hexahydrobenzo[c][1,7]naphthyridin-1-yl)-3-(3-(difluoromethyl)-4-fluorophenyl)-1-methylurea;
(R)—N-(8,9-difluoro-6-oxo-1,4,5,6-tetrahydro-2H-pyrano[3,4-c]isoquinolin-1-yl)-N-methylisoindoline-2-carboxamide;
(S)—N-(8,9-difluoro-6-oxo-1,4,5,6-tetrahydro-2H-pyrano[3,4-c]isoquinolin-1-yl)-N-methylisoindoline-2-carboxamide;
(R)-5-chloro-N-(8,9-difluoro-6-oxo-1,4,5,6-tetrahydro-2H-pyrano[3,4-c]isoquinolin-1-yl)-N-methylisoindoline-2-carboxamide;
(S)-5-chloro-N-(8,9-difluoro-6-oxo-1,4,5,6-tetrahydro-2H-pyrano[3,4-c]isoquinolin-1-yl)-N-methylisoindoline-2-carboxamide;
(R)-5-bromo-N-(8,9-difluoro-6-oxo-1,4,5,6-tetrahydro-2H-pyrano[3,4-c]isoquinolin-1-yl)-N-methylisoindoline-2-carboxamide;
(S)-5-bromo-N-(8,9-difluoro-6-oxo-1,4,5,6-tetrahydro-2H-pyrano[3,4-c]isoquinolin-1-yl)-N-methylisoindoline-2-carboxamide;
(R)-5-fluoro-N-(8,9-difluoro-6-oxo-1,4,5,6-tetrahydro-2H-pyrano[3,4-c]isoquinolin-1-yl)-N-methylisoindoline-2-carboxamide;
(S)-5-fluoro-N-(8,9-difluoro-6-oxo-1,4,5,6-tetrahydro-2H-pyrano[3,4-c]isoquinolin-1-yl)-N-methylisoindoline-2-carboxamide;
(R)—N-(8,9-difluoro-6-oxo-1,2,3,4,5,6-hexahydrobenzo[c][1,7]naphthyridin-1-yl)-N-methylisoindoline-2-carboxamide;
(S)—N-(8,9-difluoro-6-oxo-1,2,3,4,5,6-hexahydrobenzo[c][1,7]naphthyridin-1-yl)-N-methylisoindoline-2-carboxamide;
(R)—N-(8,9-difluoro-6-oxo-1,2,3,4,5,6-hexahydrobenzo[c][1,7]naphthyridin-1-yl)-5-fluoro-N-methylisoindoline-2-carboxamide;
(S)—N-(8,9-difluoro-6-oxo-1,2,3,4,5,6-hexahydrobenzo[c][1,7]naphthyridin-1-yl)-5-fluoro-N-methylisoindoline-2-carboxamide;
(R)—N-(8,9-difluoro-6-oxo-1,2,3,4,5,6-hexahydrobenzo[c][1,7]naphthyridin-1-yl)-5-chloro-N-methylisoindoline-2-carboxamide;
(S)—N-(8,9-difluoro-6-oxo-1,2,3,4,5,6-hexahydrobenzo[c][1,7]naphthyridin-1-yl)-5-chloro-N-methylisoindoline-2-carboxamide;
(R)—N-(8,9-difluoro-6-oxo-1,2,3,4,5,6-hexahydrobenzo[c][1,7]naphthyridin-1-yl)-5-bromo-N-methylisoindoline-2-carboxamide;
(S)—N-(8,9-difluoro-6-oxo-1,2,3,4,5,6-hexahydrobenzo[c][1,7]naphthyridin-1-yl)-5-bromo-N-methylisoindoline-2-carboxamide;
(R)—N-(8,9-difluoro-6-oxo-1,4,5,6-tetrahydro-2H-pyrano[3,4-c]isoquinolin-1-yl)-N-methyl-5-(trifluoromethyl)isoindoline-2-carboxamide;
(S)—N-(8,9-difluoro-6-oxo-1,4,5,6-tetrahydro-2H-pyrano[3,4-c]isoquinolin-1-yl)-N-methyl-5-(trifluoromethyl)isoindoline-2-carboxamide;
(R)—N-(8,9-difluoro-6-oxo-1,2,3,4,5,6-hexahydrobenzo[c][1,7]naphthyridin-1-yl)-N-methyl-5-(trifluoromethyl)isoindoline-2-carboxamide;
(S)—N-(8,9-difluoro-6-oxo-1,2,3,4,5,6-hexahydrobenzo[c][1,7]naphthyridin-1-yl)-N-methyl-5-(trifluoromethyl)isoindoline-2-carboxamide;
(R)-1-(8,9-difluoro-5-methyl-6-oxo-1,4,5,6-tetrahydro-2H-pyrano[3,4-c]isoquinolin-1-yl)-3-(3-(difluoromethyl)-4-fluorophenyl)-1-methylurea;
(S)-1-(8,9-difluoro-5-methyl-6-oxo-1,4,5,6-tetrahydro-2H-pyrano[3,4-c]isoquinolin-1-yl)-3-(3-(difluoromethyl)-4-fluorophenyl)-1-methylurea;
(R)-1-(8,9-difluoro-6-methoxy-1,4-dihydro-2H-pyrano[3,4-c]isoquinolin-1-yl)-3-(3-(difluoromethyl)-4-fluorophenyl)-1-methylurea;
(S)-1-(8,9-difluoro-6-methoxy-1,4-dihydro-2H-pyrano[3,4-c]isoquinolin-1-yl)-3-(3-(difluoromethyl)-4-fluorophenyl)-1-methylurea;
(R)-1-(8,9-difluoro-6-(methylamino)-1,4-dihydro-2H-pyrano[3,4-c]isoquinolin-1-yl)-3-(3-(difluoromethyl)-4-fluorophenyl)-1-methylurea
(S)-1-(8,9-difluoro-6-(methylamino)-1,4-dihydro-2H-pyrano[3,4-c]isoquinolin-1-yl)-3-(3-(difluoromethyl)-4-fluorophenyl)-1-methylurea;
(R)-1-(8,9-difluoro-6-((2-hydroxyethyl)amino)-1,4-dihydro-2H-pyrano[3,4-c]isoquinolin-1-yl)-3-(3-(difluoromethyl)-4-fluorophenyl)-1-methylurea;
(S)-1-(8,9-difluoro-6-((2-hydroxyethyl)amino)-1,4-dihydro-2H-pyrano[3,4-c]isoquinolin-1-yl)-3-(3-(difluoromethyl)-4-fluorophenyl)-1-methylurea;
(R)-1-(8,9-difluoro-6-((2-aminoethyl)amino)-1,4-dihydro-2H-pyrano[3,4-c]isoquinolin-1-yl)-3-(3-(difluoromethyl)-4-fluorophenyl)-1-methylurea
(S)-1-(8,9-difluoro-6-((2-aminoethyl)amino)-1,4-dihydro-2H-pyrano[3,4-c]isoquinolin-1-yl)-3-(3-(difluoromethyl)-4-fluorophenyl)-1-methylurea;
(R)-3-(3-chloro-4-fluorophenyl)-1-(8,9-difluoro-6-((2-aminoethyl)amino)-1,4-dihydro-2H-pyrano[3,4-c]isoquinolin-1-yl)-1-methylurea;
(S)-3-(3-chloro-4-fluorophenyl)-1-(8,9-difluoro-6-((2-aminoethyl)amino)-1,4-dihydro-2H-pyrano[3,4-c]isoquinolin-1-yl)-1-methylurea;
(R)-3-(3-chloro-4-fluorophenyl)-1-(8,9-difluoro-6-(methylamino)-1,4-dihydro-2H-pyrano[3,4-c]isoquinolin-1-yl)-1-methylurea;
(S)-3-(3-chloro-4-fluorophenyl)-1-(8,9-difluoro-6-(methylamino)-1,4-dihydro-2H-pyrano[3,4-c]isoquinolin-1-yl)-1-methylurea;
(R)-1-(8,9-difluoro-5-methyl-6-oxo-1,4,5,6-tetrahydro-2H-pyrano[3,4-c]isoquinolin-1-yl)-3-(3-chloro-4-fluorophenyl)-1-methylurea;
(S)-1-(8,9-difluoro-5-methyl-6-oxo-1,4,5,6-tetrahydro-2H-pyrano[3,4-c]isoquinolin-1-yl)-3-(3-chloro-4-fluorophenyl)-1-methylurea;
(R)-3-(3-chloro-4-fluorophenyl)-1-(8,9-difluoro-6-methoxy-1,4-dihydro-2H-pyrano[3,4-c]isoquinolin-1-yl)-1-methylurea;
(S)-3-(3-chloro-4-fluorophenyl)-1-(8,9-difluoro-6-methoxy-1,4-dihydro-2H-pyrano[3,4-c]isoquinolin-1-yl)-1-methylurea;
(R)-3-(3-chloro-4-fluorophenyl)-1-(8,9-difluoro-6-((2-hydroxyethyl)amino)-1,4-dihydro-2H-pyrano[3,4-c]isoquinolin-1-yl)-1-methylurea;
(S)-3-(3-chloro-4-fluorophenyl)-1-(8,9-difluoro-6-((2-hydroxyethyl)amino)-1,4-dihydro-2H-pyrano[3,4-c]isoquinolin-1-yl)-1-methylurea;
(R)-1-(8,9-difluoro-5-(2-hydroxyethyl)-6-oxo-1,4,5,6-tetrahydro-2H-pyrano[3,4-c]isoquinolin-1-yl)-3-(3-(difluoromethyl)-4-fluorophenyl)-1-methylurea;
(S)-1-(8,9-difluoro-5-(2-hydroxyethyl)-6-oxo-1,4,5,6-tetrahydro-2H-pyrano[3,4-c]isoquinolin-1-yl)-3-(3-(difluoromethyl)-4-fluorophenyl)-1-methylurea;
(R)-1-(6-(2-aminoethoxy)-8,9-difluoro-1,4-dihydro-2H-pyrano[3,4-c]isoquinolin-1-yl)-3-(3-(difluoromethyl)-4-fluorophenyl)-1-methylurea;
(S)-1-(6-(2-aminoethoxy)-8,9-difluoro-1,4-dihydro-2H-pyrano[3,4-c]isoquinolin-1-yl)-3-(3-(difluoromethyl)-4-fluorophenyl)-1-methylurea;
(R)-1-(5-(2-aminoethyl)-8,9-difluoro-6-oxo-1,4,5,6-tetrahydro-2H-pyrano[3,4-c]isoquinolin-1-yl)-3-(3-(difluoromethyl)-4-fluorophenyl)-1-methylurea;
(S)-1-(5-(2-aminoethyl)-8,9-difluoro-6-oxo-1,4,5,6-tetrahydro-2H-pyrano[3,4-c]isoquinolin-1-yl)-3-(3-(difluoromethyl)-4-fluorophenyl)-1-methylurea;
(R)-3-(3-chloro-4-fluorophenyl)-1-(8,9-difluoro-5-(2-hydroxyethyl)-6-oxo-1,4,5,6-tetrahydro-2H-pyrano[3,4-c]isoquinolin-1-yl)-1-methylurea;
(S)-3-(3-chloro-4-fluorophenyl)-1-(8,9-difluoro-5-(2-hydroxyethyl)-6-oxo-1,4,5,6-tetrahydro-2H-pyrano[3,4-c]isoquinolin-1-yl)-1-methylurea;
(R)-3-(3-chloro-4-fluorophenyl)-1-(8,9-difluoro-6-(2-hydroxyethoxy)-1,4-dihydro-2H-pyrano[3,4-c]isoquinolin-1-yl)-1-methylurea;
(S)-3-(3-chloro-4-fluorophenyl)-1-(8,9-difluoro-6-(2-hydroxyethoxy)-1,4-dihydro-2H-pyrano[3,4-c]isoquinolin-1-yl)-1-methylurea;
(R)-1-(8,9-difluoro-6-(2-hydroxyethoxy)-1,4-dihydro-2H-pyrano[3,4-c]isoquinolin-1-yl)-3-(3-(difluoromethyl)-4-fluorophenyl)-1-methylurea;
(S)-1-(8,9-difluoro-6-(2-hydroxyethoxy)-1,4-dihydro-2H-pyrano[3,4-c]isoquinolin-1-yl)-3-(3-(difluoromethyl)-4-fluorophenyl)-1-methylurea;
(R)-1-(5-(2-aminoethyl)-8,9-difluoro-6-oxo-1,4,5,6-tetrahydro-2H-pyrano[3,4-c]isoquinolin-1-yl)-3-(3-chloro-4-fluorophenyl)-1-methylurea;
(S)-1-(5-(2-aminoethyl)-8,9-difluoro-6-oxo-1,4,5,6-tetrahydro-2H-pyrano[3,4-c]isoquinolin-1-yl)-3-(3-chloro-4-fluorophenyl)-1-methylurea;
(R)-1-(6-(2-aminoethoxy)-8,9-difluoro-1,4-dihydro-2H-pyrano[3,4-c]isoquinolin-1-yl)-3-(3-chloro-4-fluorophenyl)-1-methylurea;
(S)-1-(6-(2-aminoethoxy)-8,9-difluoro-1,4-dihydro-2H-pyrano[3,4-c]isoquinolin-1-yl)-3-(3-chloro-4-fluorophenyl)-1-methylurea;
   or a salt, solvate, prodrug, isotopically labelled, stereoisomer, any mixture of stereoisomers, tautomer, and/or any mixture of tautomers thereof.

The compounds of the disclosure may possess one or more stereocenters, and each stereocenter may exist independently in either the (R)- or (S)-configuration. In certain embodiments, compounds described herein are present in optically active or racemic forms. The compounds described herein encompass racemic, optically active, regioisomeric and stereoisomeric forms, or combinations thereof that possess the therapeutically useful properties described herein. Preparation of optically active forms is achieved in any suitable manner, including, by way of non-limiting example, by resolution of the racemic form with recrystallization techniques, synthesis from optically active starting materials, chiral synthesis, or chromatographic separation using a chiral stationary phase. A compound illustrated herein by the racemic formula further represents either of the two enantiomers or any mixtures thereof, or in the case where two or more chiral centers are present, all diastereomers or any mixtures thereof.

In certain embodiments, the compounds of the disclosure exist as tautomers. All tautomers are included within the scope of the compounds recited herein.

Compounds described herein also include isotopically labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S. In certain embodiments, substitution with heavier isotopes such as deuterium affords greater chemical stability. Isotopically labeled compounds are prepared by any suitable method or by processes using an appropriate isotopically labeled reagent in place of the non-labeled reagent otherwise employed.

In certain embodiments, the compounds described herein are labeled by other means, including, but not limited to, the use of chromophores or fluorescent moieties, bioluminescent labels, or chemiluminescent labels.

In all of the embodiments provided herein, examples of suitable optional substituents are not intended to limit the scope of the claimed disclosure. The compounds of the disclosure may contain any of the substituents, or combinations of substituents, provided herein.

### Salts

The compounds described herein may form salts with acids or bases, and such salts are included in the present disclosure. The term “salts” embraces addition salts of free acids or bases that are useful within the methods of the disclosure. The term “pharmaceutically acceptable salt” refers to salts that possess toxicity profiles within a range that affords utility in pharmaceutical applications. In certain embodiments, the salts are pharmaceutically acceptable salts. Pharmaceutically unacceptable salts may nonetheless possess properties such as high crystallinity, which have utility in the practice of the present disclosure, such as for example utility in process of synthesis, purification or formulation of compounds useful within the methods of the disclosure.

Suitable pharmaceutically acceptable acid addition salts may be prepared from an inorganic acid or from an organic acid. Examples of inorganic acids include sulfate, hydrogen sulfate, hydrochloric, hydrobromic, hydriodic, nitric, carbonic, sulfuric, and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (or pamoic), methanesulfonic, ethanesulfonic, benzenesulfonic, pantothenic, sulfanilic, 2-hydroxyethanesulfonic, trifluoromethanesulfonic, p-toluenesulfonic, cyclohexylaminosulfonic, stearic, alginic, β-hydroxybutyric, salicylic, galactaric, galacturonic acid, glycerophosphonic acids and saccharin (e.g., saccharinate, saccharate). Salts may be comprised of a fraction of one, one or more than one molar equivalent of acid or base with respect to any compound of the disclosure.

Suitable pharmaceutically acceptable base addition salts of compounds of the disclosure include, for example, ammonium salts and metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N-dibenzylethylene-diamine, chloroprocaine, choline, diethanolamine, ethylenediamine, meglumine (or N-methylglucamine) and procaine. All of these salts may be prepared from the corresponding compound by reacting, for example, the appropriate acid or base with the compound.

### Combination Therapies

In one aspect, the compounds of the disclosure are useful within the methods of the disclosure in combination with one or more additional agents useful for treating HBV and/or HDV infections. These additional agents may comprise compounds or compositions identified herein, or compounds (e.g., commercially available compounds) known to treat, prevent, or reduce the symptoms of HBV and/or HDV infections.

Non-limiting examples of one or more additional agents useful for treating HBV and/or HDV infections include: (a) reverse transcriptase inhibitors; (b) capsid inhibitors; (c) cccDNA formation inhibitors; (d) RNA destabilizers; (e) oligomeric nucleotides targeted against the HBV genome; (f) immunostimulators, such as checkpoint inhibitors (e.g., PD-L1 inhibitors); and (g) GalNAc-siRNA conjugates targeted against an HBV gene transcript.

(a) Reverse Transcriptase Inhibitors

In certain embodiments, the reverse transcriptase inhibitor is a reverse-transcriptase inhibitor (NARTI or NRTI). In other embodiments, the reverse transcriptase inhibitor is a nucleotide analog reverse-transcriptase inhibitor (NtARTI or NtRTI).

Reported reverse transcriptase inhibitors include, but are not limited to, entecavir, clevudine, telbivudine, lamivudine, adefovir, and tenofovir, tenofovir disoproxil, tenofovir alafenamide, adefovir dipovoxil, (1R,2R,3R,5R)-3-(6-amino-9H-9-purinyl)-2-fluoro-5-(hydroxymethyl)-4-methylenecyclopentan-1-ol (described in U.S. Pat. No. 8,816,074, incorporated herein in its entirety by reference), emtricitabine, abacavir, elvucitabine, ganciclovir, lobucavir, famciclovir, penciclovir, and amdoxovir.

Reported reverse transcriptase inhibitors further include, but are not limited to, entecavir, lamivudine, and (1R,2R,3R,5R)-3-(6-amino-9H-9-purinyl)-2-fluoro-5-(hydroxymethyl)-4-methylenecyclopentan-1-ol.

Reported reverse transcriptase inhibitors further include, but are not limited to, a covalently bound phosphoramidate or phosphonamidate moiety of the above-mentioned reverse transcriptase inhibitors, or as described in for example U.S. Pat. No. 8,816,074, US Patent Application Publications No. US 2011/0245484 A1, and US 2008/0286230A1, all of which incorporated herein in their entireties by reference.

Reported reverse transcriptase inhibitors further include, but are not limited to, nucleotide analogs that comprise a phosphoramidate moiety, such as, for example, methyl ((((1R,3R,4R,5R)-3-(6-amino-9H-purin-9-yl)-4-fluoro-5-hydroxy-2-methylenecyclopentyl) methoxy)(phenoxy) phosphoryl)-(D or L)-alaninate and methyl ((((1R,2R,3R,4R)-3-fluoro-2-hydroxy-5-methylene-4-(6-oxo-1,6-dihydro-9H-purin-9-yl)cyclopentyl)methoxy)(phenoxy) phosphoryl)-(D or L)-alaninate. Also included are the individual diastereomers thereof, which include, for example, methyl ((R)-(((1R,3R,4R,5R)-3-(6-amino-9H-purin-9-yl)-4-fluoro-5-hydroxy-2-methylenecyclopentyl)methoxy)(phenoxy)phosphoryl)-(D or L)-alaninate and methyl ((S)-(((1R,3R,4R,5R)-3-(6-amino-9H-purin-9-yl)-4-fluoro-5-hydroxy-2-methylenecyclopentyl) methoxy)(phenoxy)phosphoryl)-(D or L)-alaninate.

Reported reverse transcriptase inhibitors further include, but are not limited to, compounds comprising a phosphonamidate moiety, such as, for example, tenofovir alafenamide, as well as those described in U.S. Patent Application Publication No. US 2008/0286230 A1, incorporated herein in its entirety by reference. Methods for preparing stereoselective phosphoramidate or phosphonamidate containing actives are described in, for example, U.S. Pat. No. 8,816,074, as well as U.S. Patent Application Publications No. US 2011/0245484 A1 and US 2008/0286230 A1, all of which incorporated herein in their entireties by reference.

(b) Capsid Inhibitors

As described herein, the term “capsid inhibitor” includes compounds that are capable of inhibiting the expression and/or function of a capsid protein either directly or indirectly. For example, a capsid inhibitor may include, but is not limited to, any compound that inhibits capsid assembly, induces formation of non-capsid polymers, promotes excess capsid assembly or misdirected capsid assembly, affects capsid stabilization, and/or inhibits encapsidation of RNA (pgRNA). Capsid inhibitors also include any compound that inhibits capsid function in a downstream event(s) within the replication process (e.g., viral DNA synthesis, transport of relaxed circular DNA (rcDNA) into the nucleus, covalently closed circular DNA (cccDNA) formation, virus maturation, budding and/or release, and the like). For example, in certain embodiments, the inhibitor detectably inhibits the expression level or biological activity of the capsid protein as measured, e.g., using an assay described herein. In certain embodiments, the inhibitor inhibits the level of rcDNA and downstream products of viral life cycle by at least 5%, at least 10%, at least 20%, at least 50%, at least 75%, or at least 90%.

Reported capsid inhibitors include, but are not limited to, compounds described in International Patent Applications Publication Nos WO 2013006394, WO 2014106019, and WO2014089296, all of which incorporated herein in their entireties by reference.

Reported capsid inhibitors also include, but are not limited to, the following compounds and pharmaceutically acceptable salts and/or solvates thereof: Bay-41-4109 (see Int'l Patent Application Publication No. WO 2013144129), AT-61 (see Int'l Patent Application Publication No. WO 1998033501; and King, et al., 1998, Antimicrob. Agents Chemother. 42(12):3179-3186), DVR-01 and DVR-23 (see Int'l Patent Application Publication No. WO 2013006394; and Campagna, et al., 2013, J. Virol. 87(12):6931, all of which incorporated herein in their entireties by reference.

In addition, reported capsid inhibitors include, but are not limited to, those generally and specifically described in U.S. Patent Application Publication Nos. US 2015/0225355, US 2015/0132258, US 2016/0083383, US 2016/0052921, US 2019/0225593, and Int'l Patent Application Publication Nos. WO 2013096744, WO 2014165128, WO 2014033170, WO 2014033167, WO 2014033176, WO 2014131847, WO 2014161888, WO 2014184350, WO 2014184365, WO 2015059212, WO 2015011281, WO 2015118057, WO 2015109130, WO 2015073774, WO 2015180631, WO 2015138895, WO 2016089990, WO 2017015451, WO 2016183266, WO 2017011552, WO 2017048950, WO2017048954, WO 2017048962, WO 2017064156, WO 2018052967, WO 2018172852, WO 2020023710 and are incorporated herein in their entirety by reference.

(c) cccDNA Formation Inhibitors

Covalently closed circular DNA (cccDNA) is generated in the cell nucleus from viral rcDNA and serves as the transcription template for viral mRNAs. As described herein, the term “cccDNA formation inhibitor” includes compounds that are capable of inhibiting the formation and/or stability of cccDNA either directly or indirectly. For example, a cccDNA formation inhibitor may include, but is not limited to, any compound that inhibits capsid disassembly, rcDNA entry into the nucleus, and/or the conversion of rcDNA into cccDNA. For example, in certain embodiments, the inhibitor detectably inhibits the formation and/or stability of the cccDNA as measured, e.g., using an assay described herein. In certain embodiments, the inhibitor inhibits the formation and/or stability of cccDNA by at least 5%, at least 10%, at least 20%, at least 50%, at least 75%, or at least 90%.

Reported cccDNA formation inhibitors include, but are not limited to, compounds described in Int'l Patent Application Publication No. WO 2013130703, and are incorporated herein in their entirety by reference.

In addition, reported cccDNA formation inhibitors include, but are not limited to, those generally and specifically described in U.S. Patent Application Publication No. US 2015/0038515 A1, and are incorporated herein in their entirety by reference.

(d) RNA Destabilizer

As used herein, the term “RNA destabilizer” refers to a molecule, or a salt or solvate thereof, that reduces the total amount of HBV RNA in mammalian cell culture or in a live human subject. In a non-limiting example, an RNA destabilizer reduces the amount of the RNA transcript(s) encoding one or more of the following HBV proteins: surface antigen, core protein, RNA polymerase, and e antigen. In certain embodiments, the RNA destabilizer reduces the total amount of HBV RNA in mammalian cell culture or in a live human subject by at least 5%, at least 10%, at least 20%, at least 50%, at least 75%, or at least 90%.

Reported RNA destabilizers include compounds described in U.S. Pat. No. 8,921,381, as well as compounds described in U.S. Patent Application Publication Nos. US 2015/0087659 and US 2013/0303552, all of which are incorporated herein in their entireties by reference.

In addition, reported RNA destabilizers include, but are not limited to, those generally and specifically described in Int'l Patent Application Publication Nos. WO 2015113990, WO 2015173164, US 2016/0122344, WO 2016107832, WO 2016023877, WO 2016128335, WO 2016177655, WO 2016071215, WO 2017013046, WO 2017016921, WO 2017016960, WO 2017017042, WO 2017017043, WO 2017102648, WO 2017108630, WO 2017114812, WO 2017140821, WO 2018085619, and are incorporated herein in their entirety by reference.

(e) Oligomeric Nucleotides Targeted Against the HBV Genome

Reported oligomeric nucleotides targeted against the HBV genome include, but are not limited to, Arrowhead-ARC-520 (see U.S. Pat. No. 8,809,293; and Wooddell et al., 2013, Molecular Therapy 21(5):973-985, all of which incorporated herein in their entireties by reference).

In certain embodiments, the oligomeric nucleotides can be designed to target one or more genes and/or transcripts of the HBV genome. Oligomeric nucleotide targeted to the HBV genome also include, but are not limited to, isolated, double stranded, siRNA molecules, that each include a sense strand and an antisense strand that is hybridized to the sense strand. In certain embodiments, the siRNA target one or more genes and/or transcripts of the HBV genome.

(f) Immunostimulators

Checkpoint Inhibitors

As described herein, the term “checkpoint inhibitor” includes any compound that is capable of inhibiting immune checkpoint molecules that are regulators of the immune system (e.g., stimulate or inhibit immune system activity). For example, some checkpoint inhibitors block inhibitory checkpoint molecules, thereby stimulating immune system function, such as stimulation of T cell activity against cancer cells. A non-limiting example of a checkpoint inhibitor is a PD-L1 inhibitor.

As described herein, the term “PD-L1 inhibitor” includes any compound that is capable of inhibiting the expression and/or function of the protein Programmed Death-Ligand 1 (PD-L1) either directly or indirectly. PD-L1, also known as cluster of differentiation 274 (CD274) or B7 homolog 1 (B7-H1), is a type 1 transmembrane protein that plays a major role in suppressing the adaptive arm of immune system during pregnancy, tissue allograft transplants, autoimmune disease, and hepatitis. PD-L1 binds to its receptor, the inhibitory checkpoint molecule PD-1 (which is found on activated T cells, B cells, and myeloid cells) so as to modulate activation or inhibition of the adaptive arm of immune system. In certain embodiments, the PD-L1 inhibitor inhibits the expression and/or function of PD-L1 by at least 5%, at least 10%, at least 20%, at least 50%, at least 75%, or at least 90%.

Reported PD-L1 inhibitors include, but are not limited to, compounds recited in one of the following patent application publications: US 2018/0057455; US 2018/0057486; WO 2017/106634; WO 2018/026971; WO 2018/045142; WO 2018/118848; WO 2018/119221; WO 2018/119236; WO 2018/119266; WO 2018/119286; WO 2018/121560; WO 2019/076343; WO 2019/087214; and are incorporated herein in their entirety by reference.

(g) GalNAc-siRNA Conjugates Targeted Against an HBV Gene Transcript

“GalNAc” is the abbreviation for N-acetylgalactosamine, and “siRNA” is the abbreviation for small interfering RNA. An siRNA that targets an HBV gene transcript is covalently bonded to GalNAc in a GalNAc-siRNA conjugate useful in the practice of the present disclosure. While not wishing to be bound by theory, it is believed that GalNAc binds to asialoglycoprotein receptors on hepatocytes thereby facilitating the targeting of the siRNA to the hepatocytes that are infected with HBV. The siRNA enter the infected hepatocytes and stimulate destruction of HBV gene transcripts by the phenomenon of RNA interference.

Examples of GalNAc-siRNA conjugates useful in the practice of this aspect of the present disclosure are set forth in published international application PCT/CA2017/050447 (PCT Application Publication number WO/2017/177326, published on Oct. 19, 2017) which is hereby incorporated by reference in its entirety.

A synergistic effect may be calculated, for example, using suitable methods such as, for example, the Sigmoid-Eₘₐₓ equation (Holford & Scheiner, 1981, Clin. Pharmacokinet. 6:429-453), the equation of Loewe additivity (Loewe & Muischnek, 1926, Arch. Exp. Pathol Pharmacol. 114: 313-326) and the median-effect equation (Chou & Talalay, 1984, Adv. Enzyme Regul. 22:27-55). Each equation referred to elsewhere herein may be applied to experimental data to generate a corresponding graph to aid in assessing the effects of the drug combination. The corresponding graphs associated with the equations referred to elsewhere herein are the concentration-effect curve, isobologram curve and combination index curve, respectively.

### Synthesis

The present disclosure further provides methods of preparing compounds of the present disclosure. Compounds of the present teachings can be prepared in accordance with the procedures outlined herein, from commercially available starting materials, compounds known in the literature, or readily prepared intermediates, by employing standard synthetic methods and procedures known to those skilled in the art. Standard synthetic methods and procedures for the preparation of organic molecules and functional group transformations and manipulations can be readily obtained from the relevant scientific literature or from standard textbooks in the field.

It is appreciated that where typical or preferred process conditions (i.e., reaction temperatures, times, mole ratios of reactants, solvents, pressures, and so forth) are given, other process conditions can also be used unless otherwise stated. Optimum reaction conditions can vary with the particular reactants or solvent used, but such conditions can be determined by one skilled in the art by routine optimization procedures. Those skilled in the art of organic synthesis will recognize that the nature and order of the synthetic steps presented can be varied for the purpose of optimizing the formation of the compounds described herein.

The processes described herein can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry, or by chromatography such as high-performance liquid chromatography (HPLC), gas chromatography (GC), gel-permeation chromatography (GPC), or thin layer chromatography (TLC).

Preparation of the compounds can involve protection and deprotection of various chemical groups. The need for protection and deprotection and the selection of appropriate protecting groups can be readily determined by one skilled in the art. The chemistry of protecting groups can be found, for example, in Greene, et al., Protective Groups in Organic Synthesis, 2d. Ed. (Wiley & Sons, 1991), the entire disclosure of which is incorporated by reference herein for all purposes.

The reactions or the processes described herein can be carried out in suitable solvents that can be readily selected by one skilled in the art of organic synthesis. Suitable solvents typically are substantially nonreactive with the reactants, intermediates, and/or products at the temperatures at which the reactions are carried out, i.e., temperatures that can range from the solvent's freezing temperature to the solvent's boiling temperature. A given reaction can be carried out in one solvent or a mixture of more than one solvent. Depending on the particular reaction step, suitable solvents for a particular reaction step can be selected.

A compound of formula (I) can be prepared from commercially available or previously documented starting materials, for example, according to the synthetic methods outlined in Scheme 1 (wherein, in certain embodiments, Y is O or NH).

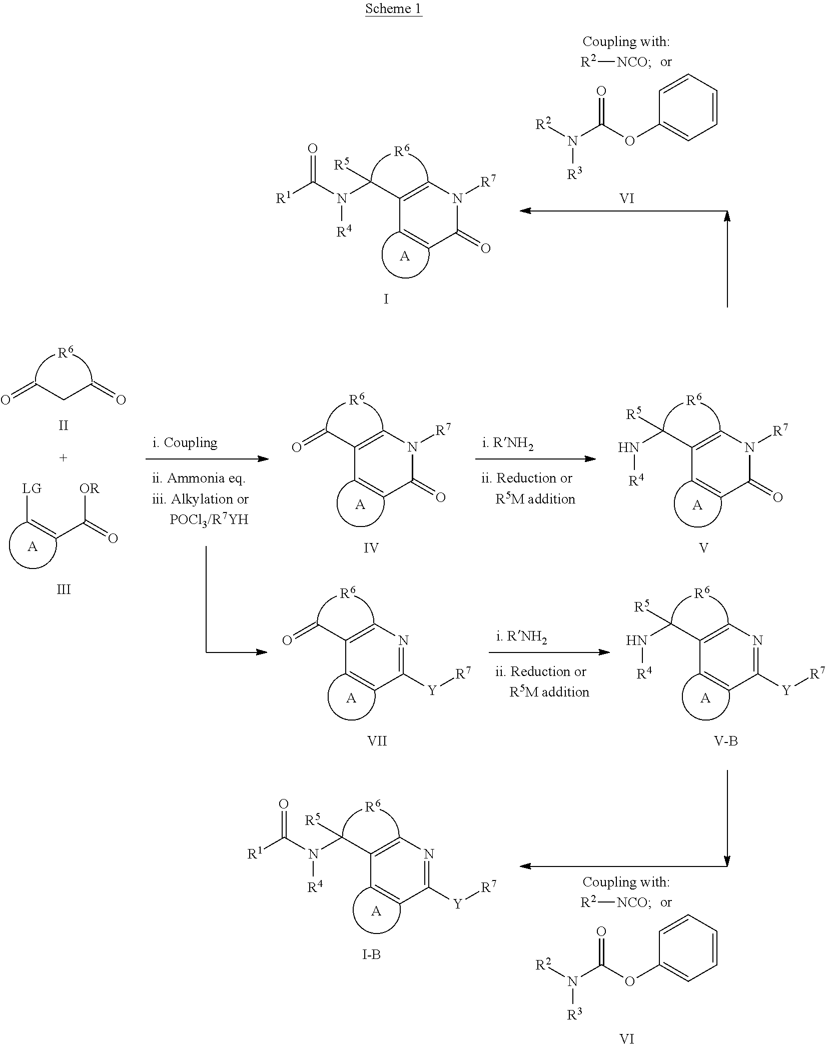

Bi- or tri-cyclic ketones IV can be prepared from 1,3-diketones II and carboxylic acid derivatives III by a coupling reaction (when LG in III is, in non-limiting examples, a halogen or a TfO— group) in the presence of a metal catalyst such as, but not limited to, copper iodide, or by an aldol-type condensation (when III is a β-ketoacid or β-ketoester), followed by reaction of the generated intermediates, either isolated or in situ, with ammonia or amines and then optionally by alkylation. In the latter case, O-alkylation provides ketone VII. N-Alkylated ketones VII (Y═NH) can also be prepared from ketones IV (with R⁷═H) by treatment with, in a non-limiting example, POCl₃, followed by nucleophilic displacement of the resulting chlorides with appropriate ammonia or amines. Ketones IV and VII are condensed with amines and the resulting intermediate imines are reacted with a reducing agent, such as but not limited to sodium borohydride, or carbon-based nucleophiles, such as but not limited to a Grignard reagent or an alkyl/aryl lithium reagent to afford amines V, or V-B. In certain embodiments, the primary R′NH₂ amine can be racemic, scalemic, or enantiopure, and can be used to influence the stereochemical outcome of the imine reduction or carbon-based nucleophile addition. The resulting secondary amine can be further reacted with an aldehyde and a reducing agent such as but not limited to sodium triacetoxyborohydride, and the R′ group can be removed to provide V, or V-B. Alternatively, IV and VII can be reacted with a primary sulfinamide to form a sulfinimine, which is subsequently reacted with a reducing agent, such as but not limited to sodium borohydride, or a carbon-based nucleophile, such as but not limited to a Grignard reagent or an alkyl/aryl lithium. In certain embodiments, the primary sulfinamide can be racemic, scalemic, or enantiopure, and can be used to influence the stereochemical outcome of the sulfinimine reduction. The resulting secondary sulfinamide can be further functionalized with an electrophile, such as but not limited to an alkyl halide, in the presence of base, such as but not limited to sodium hydride, and the sulfonamido group can be removed to provide V, or V-B. Functionalization of V or V-B with a variety of electrophiles, for example an isocyanate or a phenyl carbamate VI, provides I, or I-B.

The protocols incorporated elsewhere herein exemplify synthesis of representative compounds of the present disclosure. Analogous compounds can be synthesized in a similar fashion to those exemplified using the appropriately substituted intermediates and reagents.

### Methods

The disclosure provides a method of treating, ameliorating, and/or preventing hepatitis virus infection in a subject. In certain embodiments, the infection comprises hepatitis B virus (HBV) infection. In other embodiments, the method comprises administering to the subject in need thereof a therapeutically effective amount of at least one compound and/or composition of the disclosure. In yet other embodiments, the at least one compound of the disclosure is the only antiviral agent administered to the subject. In yet other embodiments, the at least one compound is administered to the subject in a pharmaceutically acceptable composition. In yet other embodiments, the subject is further administered at least one additional agent useful for treating the hepatitis infection. In yet other embodiments, the at least one additional agent comprises at least one selected from the group consisting of reverse transcriptase inhibitor; capsid inhibitor; cccDNA formation inhibitor; RNA destabilizer; oligomeric nucleotide targeted against the HBV genome; immunostimulator, such as checkpoint inhibitor (e.g., PD-L1 inhibitor); and GalNAc-siRNA conjugate targeted against an HBV gene transcript. In yet other embodiments, the subject is co-administered the at least one compound and the at least one additional agent. In yet other embodiments, the at least one compound and the at least one additional agent are coformulated.

The disclosure further provides a method of inhibiting expression and/or function of a viral capsid protein either directly or indirectly in a subject. In certain embodiments, the method comprises administering to the subject in need thereof a therapeutically effective amount of at least one compound and/or composition of the disclosure. In other embodiments, the at least one compound is administered to the subject in a pharmaceutically acceptable composition. In yet other embodiments, the at least one compound of the disclosure is the only antiviral agent administered to the subject. In yet other embodiments, the subject is further administered at least one additional agent useful for treating HBV infection. In yet other embodiments, the at least one additional agent comprises at least one selected from the group consisting of reverse transcriptase inhibitor; capsid inhibitor; cccDNA formation inhibitor; RNA destabilizer; oligomeric nucleotide targeted against the HBV genome; immunostimulator, such as checkpoint inhibitor (e.g., PD-L1 inhibitor); and GalNAc-siRNA conjugate targeted against an HBV gene transcript. In yet other embodiments, the subject is co-administered the at least one compound and the at least one additional agent. In yet other embodiments, the at least one compound and the at least one additional agent are coformulated.

In certain embodiments, the subject is a mammal. In other embodiments, the mammal is a human.

### Pharmaceutical Compositions and Formulations

The disclosure provides pharmaceutical compositions comprising at least one compound of the disclosure or a salt or solvate thereof, which are useful to practice methods of the disclosure. Such a pharmaceutical composition may consist of at least one compound of the disclosure or a salt or solvate thereof, in a form suitable for administration to a subject, or the pharmaceutical composition may comprise at least one compound of the disclosure or a salt or solvate thereof, and one or more pharmaceutically acceptable carriers, one or more additional ingredients, or any combinations of these. At least one compound of the disclosure may be present in the pharmaceutical composition in the form of a physiologically acceptable salt, such as in combination with a physiologically acceptable cation or anion, as is well known in the art.

In certain embodiments, the pharmaceutical compositions useful for practicing the method of the disclosure may be administered to deliver a dose of between 1 ng/kg/day and 100 mg/kg/day. In other embodiments, the pharmaceutical compositions useful for practicing the disclosure may be administered to deliver a dose of between 1 ng/kg/day and 1,000 mg/kg/day.

The relative amounts of the active ingredient, the pharmaceutically acceptable carrier, and any additional ingredients in a pharmaceutical composition of the disclosure will vary, depending upon the identity, size, and condition of the subject treated and further depending upon the route by which the composition is to be administered. By way of example, the composition may comprise between 0.1% and 100% (w/w) active ingredient.

Pharmaceutical compositions that are useful in the methods of the disclosure may be suitably developed for nasal, inhalational, oral, rectal, vaginal, pleural, peritoneal, parenteral, topical, transdermal, pulmonary, intranasal, buccal, ophthalmic, epidural, intrathecal, intravenous, or another route of administration. A composition useful within the methods of the disclosure may be directly administered to the brain, the brainstem, or any other part of the central nervous system of a mammal or bird. Other contemplated formulations include projected nanoparticles, microspheres, liposomal preparations, coated particles, polymer conjugates, resealed erythrocytes containing the active ingredient, and immunologically-based formulations.

In certain embodiments, the compositions of the disclosure are part of a pharmaceutical matrix, which allows for manipulation of insoluble materials and improvement of the bioavailability thereof, development of controlled or sustained release products, and generation of homogeneous compositions. By way of example, a pharmaceutical matrix may be prepared using hot melt extrusion, solid solutions, solid dispersions, size reduction technologies, molecular complexes (e.g., cyclodextrins, and others), microparticulate, and particle and formulation coating processes. Amorphous or crystalline phases may be used in such processes.

The route(s) of administration will be readily apparent to the skilled artisan and will depend upon any number of factors including the type and severity of the disease being treated, the type and age of the veterinary or human patient being treated, and the like.

The formulations of the pharmaceutical compositions described herein may be prepared by any method known or hereafter developed in the art of pharmacology and pharmaceutics. In general, such preparatory methods include the step of bringing the active ingredient into association with a carrier or one or more other accessory ingredients, and then, if necessary or desirable, shaping or packaging the product into a desired single-dose or multi-dose unit.

As used herein, a “unit dose” is a discrete amount of the pharmaceutical composition comprising a predetermined amount of the active ingredient. The amount of the active ingredient is generally equal to the dosage of the active ingredient that would be administered to a subject or a convenient fraction of such a dosage such as, for example, one-half or one-third of such a dosage. The unit dosage form may be for a single daily dose or one of multiple daily doses (e.g., about 1 to 4 or more times per day). When multiple daily doses are used, the unit dosage form may be the same or different for each dose.

Although the descriptions of pharmaceutical compositions provided herein are principally directed to pharmaceutical compositions suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the disclosure is contemplated include, but are not limited to, humans and other primates, mammals including commercially relevant mammals such as cattle, pigs, horses, sheep, cats, and dogs.

In certain embodiments, the compositions of the disclosure are formulated using one or more pharmaceutically acceptable excipients or carriers. In certain embodiments, the pharmaceutical compositions of the disclosure comprise a therapeutically effective amount of at least one compound of the disclosure and a pharmaceutically acceptable carrier. Pharmaceutically acceptable carriers, which are useful, include, but are not limited to, glycerol, water, saline, ethanol, recombinant human albumin (e.g., RECOMBUMIN®), solubilized gelatins (e.g., GELOFUSINE®), and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey).

The carrier may be a solvent or dispersion medium containing, for example, water, ethanol, polyol (for example, glycerol, propylene glycol, and liquid polyethylene glycol, and the like), recombinant human albumin, solubilized gelatins, suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, isotonic agents, for example, sugars, sodium chloride, or polyalcohols such as mannitol and sorbitol, are included in the composition. Prolonged absorption of the injectable compositions may be brought about by including in the composition an agent that delays absorption, for example, aluminum monostearate or gelatin.

Formulations may be employed in admixtures with conventional excipients, i.e., pharmaceutically acceptable organic or inorganic carrier substances suitable for oral, parenteral, nasal, inhalational, intravenous, subcutaneous, transdermal enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring, and/or fragrance-conferring substances and the like. They may also be combined where desired with other active agents, e.g., other analgesic, anxiolytics or hypnotic agents. As used herein, “additional ingredients” include, but are not limited to, one or more ingredients that may be used as a pharmaceutical carrier.

The composition of the disclosure may comprise a preservative from about 0.005% to 2.0% by total weight of the composition. The preservative is used to prevent spoilage in the case of exposure to contaminants in the environment. Examples of preservatives useful in accordance with the disclosure include but are not limited to those selected from the group consisting of benzyl alcohol, sorbic acid, parabens, imidurea and any combinations thereof. One such preservative is a combination of about 0.5% to 2.0% benzyl alcohol and 0.05-0.5% sorbic acid.

The composition may include an antioxidant and a chelating agent that inhibit the degradation of the compound. Antioxidants for some compounds are BHT, BHA, alpha-tocopherol and ascorbic acid in the exemplary range of about 0.01% to 0.3%, or BHT in the range of 0.03% to 0.1% by weight by total weight of the composition. The chelating agent may be present in an amount of from 0.01% to 0.5% by weight by total weight of the composition. Exemplary chelating agents include edetate salts (e.g. disodium edetate) and citric acid in the weight range of about 0.01% to 0.20%, or in the range of 0.02% to 0.10% by weight by total weight of the composition. The chelating agent is useful for chelating metal ions in the composition that may be detrimental to the shelf life of the formulation. While BHT and disodium edetate are exemplary antioxidant and chelating agent, respectively, for some compounds, other suitable and equivalent antioxidants and chelating agents may be substituted therefore as would be known to those skilled in the art.

Liquid suspensions may be prepared using conventional methods to achieve suspension of the active ingredient in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethyl cellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, acacia, and ionic or non-ionic surfactants. Known preservatives include, but are not limited to, methyl, ethyl, or n-propyl para-hydroxybenzoates, ascorbic acid, and sorbic acid. Known sweetening agents include, for example, glycerol, propylene glycol, sorbitol, sucrose, and saccharin.

Liquid solutions of the active ingredient in aqueous or oily solvents may be prepared in substantially the same manner as liquid suspensions, the primary difference being that the active ingredient is dissolved, rather than suspended in the solvent. As used herein, an “oily” liquid is one which comprises a carbon-containing liquid molecule and which exhibits a less polar character than water. Liquid solutions of the pharmaceutical composition of the disclosure may comprise each of the components described with regard to liquid suspensions, it being understood that suspending agents will not necessarily aid dissolution of the active ingredient in the solvent. Aqueous solvents include, for example, water, and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the disclosure may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, ionic and non-ionic surfactants, and a preservative. Additional excipients, such as fillers and sweetening, flavoring, or coloring agents, may also be included in these formulations.

A pharmaceutical composition of the disclosure may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying. Methods for mixing components include physical milling, the use of pellets in solid and suspension formulations and mixing in a transdermal patch, as known to those skilled in the art.

### Administration/Dosing

The regimen of administration may affect what constitutes an effective amount. The therapeutic formulations may be administered to the patient either prior to or after the onset of a disease or disorder. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present disclosure to a patient, such as a mammal, such as a human, may be carried out using known procedures, at dosages and for periods of time effective to treat a disease or disorder contemplated herein. An effective amount of the therapeutic compound necessary to achieve a therapeutic effect may vary according to factors such as the activity of the particular compound employed; the time of administration; the rate of excretion of the compound; the duration of the treatment; other drugs, compounds or materials used in combination with the compound; the state of the disease or disorder, age, sex, weight, condition, general health and prior medical history of the patient being treated, and like factors well-known in the medical arts. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the disclosure is from about 0.01 mg/kg to 100 mg/kg of body weight/per day. One of ordinary skill in the art would be able to study the relevant factors and make the determination regarding the effective amount of the therapeutic compound without undue experimentation.

The compound may be administered to an animal as frequently as several times daily, or it may be administered less frequently, such as once a day, once a week, once every two weeks, once a month, or even less frequently, such as once every several months or even once a year or less. It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on. The frequency of the dose is readily apparent to the skilled artisan and depends upon a number of factors, such as, but not limited to, type and severity of the disease being treated, and type and age of the animal.

Actual dosage levels of the active ingredients in the pharmaceutical compositions of this disclosure may be varied so as to obtain an amount of the active ingredient that is effective to achieve the desired therapeutic response for a particular patient, composition, and mode of administration, without being toxic to the patient.

A medical doctor, e.g., physician or veterinarian, having ordinary skill in the art may readily determine and prescribe the effective amount of the pharmaceutical composition required. For example, the physician or veterinarian could start doses of the compounds of the disclosure employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the disclosure are dictated by and directly dependent on (a) the unique characteristics of the therapeutic compound and the particular therapeutic effect to be achieved, and (b) the limitations inherent in the art of compounding/formulating such a therapeutic compound for the treatment of a disease or disorder in a patient.

In certain embodiments, the compositions of the disclosure are administered to the patient in dosages that range from one to five times per day or more. In other embodiments, the compositions of the disclosure are administered to the patient in range of dosages that include, but are not limited to, once every day, every two days, every three days to once a week, and once every two weeks. It will be readily apparent to one skilled in the art that the frequency of administration of the various combination compositions of the disclosure will vary from subject to subject depending on many factors including, but not limited to, age, disease or disorder to be treated, gender, overall health, and other factors. Thus, the disclosure should not be construed to be limited to any particular dosage regime and the precise dosage and composition to be administered to any patient will be determined by the attending physician taking all other factors about the patient into account.

Compounds of the disclosure for administration may be in the range of from about 1 pg to about 7,500 mg, about 20 μg to about 7,000 mg, about 40 μg to about 6,500 mg, about 80 μg to about 6,000 mg, about 100 μg to about 5,500 mg, about 200 μg to about 5,000 mg, about 400 μg to about 4,000 mg, about 800 μg to about 3,000 mg, about 1 mg to about 2,500 mg, about 2 mg to about 2,000 mg, about 5 mg to about 1,000 mg, about 10 mg to about 750 mg, about 20 mg to about 600 mg, about 30 mg to about 500 mg, about 40 mg to about 400 mg, about 50 mg to about 300 mg, about 60 mg to about 250 mg, about 70 mg to about 200 mg, about 80 mg to about 150 mg, and any and all whole or partial increments there-in-between.

In some embodiments, the dose of a compound of the disclosure is from about 0.5 μg and about 5,000 mg. In some embodiments, a dose of a compound of the disclosure used in compositions described herein is less than about 5,000 mg, or less than about 4,000 mg, or less than about 3,000 mg, or less than about 2,000 mg, or less than about 1,000 mg, or less than about 800 mg, or less than about 600 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, the present disclosure is directed to a packaged pharmaceutical composition comprising a container holding a therapeutically effective amount of a compound of the disclosure, alone or in combination with a second pharmaceutical agent; and instructions for using the compound to treat, prevent, or reduce one or more symptoms of a disease or disorder in a patient.

The term “container” includes any receptacle for holding the pharmaceutical composition or for managing stability or water uptake. For example, in certain embodiments, the container is the packaging that contains the pharmaceutical composition, such as liquid (solution and suspension), semisolid, lyophilized solid, solution and powder or lyophilized formulation present in dual chambers. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions may contain information pertaining to the compound's ability to perform its intended function, e.g., treating, preventing, or reducing a disease or disorder in a patient.

### Administration

Routes of administration of any of the compositions of the disclosure include inhalational, oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, epidural, intrapleural, intraperitoneal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, emulsions, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present disclosure are not limited to the particular formulations and compositions that are described herein.

### Oral Administration

For oral application, particularly suitable are tablets, dragees, liquids, drops, capsules, caplets and gelcaps. Other formulations suitable for oral administration include, but are not limited to, a powdered or granular formulation, an aqueous or oily suspension, an aqueous or oily solution, a paste, a gel, toothpaste, a mouthwash, a coating, an oral rinse, or an emulsion. The compositions intended for oral use may be prepared according to any method known in the art and such compositions may contain one or more agents selected from the group consisting of inert, non-toxic, generally recognized as safe (GRAS) pharmaceutically excipients which are suitable for the manufacture of tablets. Such excipients include, for example an inert diluent such as lactose; granulating and disintegrating agents such as cornstarch; binding agents such as starch; and lubricating agents such as magnesium stearate.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and U.S. Pat. No. 4,265,874 to form osmotically controlled release tablets. Tablets may further comprise a sweetening agent, a flavoring agent, a coloring agent, a preservative, or some combination of these in order to provide for pharmaceutically elegant and palatable preparation. Hard capsules comprising the active ingredient may be made using a physiologically degradable composition, such as gelatin. The capsules comprise the active ingredient, and may further comprise additional ingredients including, for example, an inert solid diluent such as calcium carbonate, calcium phosphate, or kaolin.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin from animal-derived collagen or from a hypromellose, a modified form of cellulose, and manufactured using optional mixtures of gelatin, water and plasticizers such as sorbitol or glycerol. Such soft capsules comprise the active ingredient, which may be mixed with water or an oil medium such as peanut oil, liquid paraffin, or olive oil.

For oral administration, the compounds of the disclosure may be in the form of tablets or capsules prepared by conventional means with pharmaceutically acceptable excipients such as binding agents; fillers; lubricants; disintegrates; or wetting agents. If desired, the tablets may be coated using suitable methods and coating materials such as OPADRY® film coating systems available from Colorcon, West Point, Pa. (e.g., OPADRY® OY Type, OYC Type, Organic Enteric OY-P Type, Aqueous Enteric OY-A Type, OY-PM Type and OPADRY® White, 32K18400). It is understood that similar type of film coating or polymeric products from other companies may be used.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface-active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycolate. Known surface-active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Granulating techniques are well known in the pharmaceutical art for modifying starting powders or other particulate materials of an active ingredient. The powders are typically mixed with a binder material into larger permanent free-flowing agglomerates or granules referred to as a “granulation.” For example, solvent-using “wet” granulation processes are generally characterized in that the powders are combined with a binder material and moistened with water or an organic solvent under conditions resulting in the formation of a wet granulated mass from which the solvent must then be evaporated.

Melt granulation generally consists in the use of materials that are solid or semi-solid at room temperature (i.e., having a relatively low softening or melting point range) to promote granulation of powdered or other materials, essentially in the absence of added water or other liquid solvents. The low melting solids, when heated to a temperature in the melting point range, liquefy to act as a binder or granulating medium. The liquefied solid spreads itself over the surface of powdered materials with which it is contacted, and on cooling, forms a solid granulated mass in which the initial materials are bound together. The resulting melt granulation may then be provided to a tablet press or be encapsulated for preparing the oral dosage form. Melt granulation improves the dissolution rate and bioavailability of an active (i.e., drug) by forming a solid dispersion or solid solution.

U.S. Pat. No. 5,169,645 discloses directly compressible wax-containing granules having improved flow properties. The granules are obtained when waxes are admixed in the melt with certain flow improving additives, followed by cooling and granulation of the admixture. In certain embodiments, only the wax itself melts in the melt combination of the wax(es) and additives(s), and in other cases both the wax(es) and the additives(s) will melt.

The present disclosure also includes a multi-layer tablet comprising a layer providing for the delayed release of one or more compounds useful within the methods of the disclosure, and a further layer providing for the immediate release of one or more compounds useful within the methods of the disclosure. Using a wax/pH-sensitive polymer mix, a gastric insoluble composition may be obtained in which the active ingredient is entrapped, ensuring its delayed release.

Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl para-hydroxy benzoates or sorbic acid). Liquid formulations of a pharmaceutical composition of the disclosure which are suitable for oral administration may be prepared, packaged, and sold either in liquid form or in the form of a dry product intended for reconstitution with water or another suitable vehicle prior to use.

### Parenteral Administration

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intravenous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multidose containers containing a preservative. Injectable formulations may also be prepared, packaged, or sold in devices such as patient-controlled analgesia (PCA) devices. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally acceptable diluent or solvent, such as water or 1,3-butanediol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form in a recombinant human albumin, a fluidized gelatin, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

### Topical Administration

An obstacle for topical administration of pharmaceuticals is the stratum corneum layer of the epidermis. The stratum corneum is a highly resistant layer comprised of protein, cholesterol, sphingolipids, free fatty acids and various other lipids, and includes cornified and living cells. One of the factors that limit the penetration rate (flux) of a compound through the stratum corneum is the amount of the active substance that can be loaded or applied onto the skin surface. The greater the amount of active substance which is applied per unit of area of the skin, the greater the concentration gradient between the skin surface and the lower layers of the skin, and in turn the greater the diffusion force of the active substance through the skin. Therefore, a formulation containing a greater concentration of the active substance is more likely to result in penetration of the active substance through the skin, and more of it, and at a more consistent rate, than a formulation having a lesser concentration, all other things being equal.

Formulations suitable for topical administration include, but are not limited to, liquid or semi-liquid preparations such as liniments, lotions, oil-in-water or water-in-oil emulsions such as creams, ointments or pastes, and solutions or suspensions. Topically administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein.

Enhancers of permeation may be used. These materials increase the rate of penetration of drugs across the skin. Typical enhancers in the art include ethanol, glycerol monolaurate, PGML (polyethylene glycol monolaurate), dimethylsulfoxide, and the like. Other enhancers include oleic acid, oleyl alcohol, ethoxydiglycol, laurocapram, alkanecarboxylic acids, dimethylsulfoxide, polar lipids, or N-methyl-2-pyrrolidone.

One acceptable vehicle for topical delivery of some of the compositions of the disclosure may contain liposomes. The composition of the liposomes and their use are known in the art (i.e., U.S. Pat. No. 6,323,219).

In alternative embodiments, the topically active pharmaceutical composition may be optionally combined with other ingredients such as adjuvants, anti-oxidants, chelating agents, surfactants, foaming agents, wetting agents, emulsifying agents, viscosifiers, buffering agents, preservatives, and the like. In other embodiments, a permeation or penetration enhancer is included in the composition and is effective in improving the percutaneous penetration of the active ingredient into and through the stratum corneum with respect to a composition lacking the permeation enhancer. Various permeation enhancers, including oleic acid, oleyl alcohol, ethoxydiglycol, laurocapram, alkanecarboxylic acids, dimethylsulfoxide, polar lipids, or N-methyl-2-pyrrolidone, are known to those of skill in the art. In another aspect, the composition may further comprise a hydrotropic agent, which functions to increase disorder in the structure of the stratum corneum, and thus allows increased transport across the stratum corneum. Various hydrotropic agents such as isopropyl alcohol, propylene glycol, or sodium xylene sulfonate, are known to those of skill in the art.

The topically active pharmaceutical composition should be applied in an amount effective to affect desired changes. As used herein “amount effective” shall mean an amount sufficient to cover the region of skin surface where a change is desired. An active compound should be present in the amount of from about 0.0001% to about 15% by weight volume of the composition. For example, it should be present in an amount from about 0.0005% to about 5% of the composition; for example, it should be present in an amount of from about 0.001% to about 1% of the composition. Such compounds may be synthetically- or naturally derived.

### Buccal Administration

A pharmaceutical composition of the disclosure may be prepared, packaged, or sold in a formulation suitable for buccal administration. Such formulations may, for example, be in the form of tablets or lozenges made using conventional methods, and may contain, for example, 0.1 to 20% (w/w) of the active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, may have an average particle or droplet size in the range from about 0.1 to about 200 nanometers, and may further comprise one or more of the additional ingredients described herein. The examples of formulations described herein are not exhaustive and it is understood that the disclosure includes additional modifications of these and other formulations not described herein, but which are known to those of skill in the art.

### Rectal Administration

A pharmaceutical composition of the disclosure may be prepared, packaged, or sold in a formulation suitable for rectal administration. Such a composition may be in the form of, for example, a suppository, a retention enema preparation, and a solution for rectal or colonic irrigation.

Suppository formulations may be made by combining the active ingredient with a non-irritating pharmaceutically acceptable excipient which is solid at ordinary room temperature (i.e., about 20° C.) and which is liquid at the rectal temperature of the subject (i.e., about 37° C. in a healthy human). Suitable pharmaceutically acceptable excipients include, but are not limited to, cocoa butter, polyethylene glycols, and various glycerides. Suppository formulations may further comprise various additional ingredients including, but not limited to, antioxidants, and preservatives.

Retention enema preparations or solutions for rectal or colonic irrigation may be made by combining the active ingredient with a pharmaceutically acceptable liquid carrier. As is well known in the art, enema preparations may be administered using, and may be packaged within, a delivery device adapted to the rectal anatomy of the subject. Enema preparations may further comprise various additional ingredients including, but not limited to, antioxidants, and preservatives.

### Additional Administration Forms

Additional dosage forms of this disclosure include dosage forms as described in U.S. Pat. Nos. 6,340,475, 6,488,962, 6,451,808, 5,972,389, 5,582,837, and 5,007,790. Additional dosage forms of this disclosure also include dosage forms as described in U.S. Patent Applications Nos. 20030147952, 20030104062, 20030104053, 20030044466, 20030039688, and 20020051820. Additional dosage forms of this disclosure also include dosage forms as described in PCT Applications Nos. WO 03/35041, WO 03/35040, WO 03/35029, WO 03/35177, WO 03/35039, WO 02/96404, WO 02/32416, WO 01/97783, WO 01/56544, WO 01/32217, WO 98/55107, WO 98/11879, WO 97/47285, WO 93/18755, and WO 90/11757.

### Controlled Release Formulations and Drug Delivery Systems:

In certain embodiments, the compositions and/or formulations of the present disclosure may be, but are not limited to, short-term, rapid-onset and/or rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use the method of the disclosure may be administered in the form of microparticles, for example, by injection or in the form of wafers or discs by implantation.

In certain embodiments of the disclosure, the compounds useful within the disclosure are administered to a subject, alone or in combination with another pharmaceutical agent, using a sustained release formulation.

The term delayed release is used herein in its conventional sense to refer to a drug formulation that provides for an initial release of the drug after some delay following drug administration and that may, although not necessarily, include a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

Those skilled in the art will recognize or be able to ascertain using no more than routine experimentation, numerous equivalents to the specific procedures, embodiments, claims, and examples described herein. Such equivalents were considered to be within the scope of this disclosure and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that, wherever values and ranges are provided herein, the description in range format is merely for convenience and brevity and should not be construed as an inflexible limitation on the scope of the disclosure. Accordingly, all values and ranges encompassed by these values and ranges are meant to be encompassed within the scope of the present disclosure. Moreover, all values that fall within these ranges, as well as the upper or lower limits of a range of values, are also contemplated by the present application. The description of a range should be considered to have specifically disclosed all the possible sub-ranges as well as individual numerical values within that range and, when appropriate, partial integers of the numerical values within ranges. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

The following examples further illustrate aspects of the present disclosure. However, they are in no way a limitation of the teachings or disclosure of the present disclosure as set forth herein.

### Examples

The disclosure is now described with reference to the following Examples. These Examples are provided for the purpose of illustration only, and the disclosure is not limited to these Examples, but rather encompasses all variations that are evident as a result of the teachings provided herein.

### Materials & Methods

The following procedures can be utilized in evaluating and selecting compounds that inhibit hepatitis B virus infection.

HepDE19 Assay with bDNA Quantitation of HBV rcDNA:

HepDE19 cell culture system is a HepG2 (human hepatocarcinoma) derived cell line that supports HBV DNA replication and cccDNA formation in a tetracycline (Tet)-regulated manner and produces HBV rcDNA and a detectable reporter molecule dependent on the production and maintenance of cccDNA (Guo, et al., 2007, J. Virol. 81:12472-12484).

HepDE19 (50,000 cells/well) were plated in 96-well collagen-coated tissue-culture treated microtiter plates in DMEM/F12 medium supplemented with 10% fetal bovine serum, 1% penicillin-streptomycin and 1 μg/mL tetracycline and incubated in a humidified incubator at 37° C. and 5% CO₂ overnight. Next day, the cells were switched to fresh medium without tetracycline and incubated for 4 hours at 37° C. and 5% CO₂. The cells were treated with fresh Tet-free medium with compounds at concentrations starting at 25 μM and a serial, 12 log, 8-point, titration series in duplicate. The final DMSO concentration in the assay was 0.5%. The plates were incubated for 7 days in a humidified incubator at 37° C. and 5% CO₂. Following a 7 day-incubation, the level of rcDNA present in the inhibitor-treated wells was measured using a Quantigene 2.0 bDNA assay kit (Affymetrix, Santa Clara, Calif.) with HBV specific custom probe set and manufacturers instructions. Concurrently, the effect of compounds on cell viability was assessed using replicate plates, plated at a density of 5,000 cells/well and incubated for 4 days, to determine the ATP content as a measure of cell viability using the cell-titer glo reagent (CTG; Promega Corporation, Madison, Wis.) as per manufacturer's instructions. The plates were read using a Victor luminescence plate reader (PerkinElmer Model 1420 Multilabel counter) and the relative luminescence units (RLU) data generated from each well was calculated as % inhibition of the untreated control wells and analyzed using XL-Fit module in Microsoft Excel to determine EC₅₀ and EC₉₀ (bDNA) and CC₅₀ (CTG) values using a 4-parameter curve fitting algorithm.

### LCMS Methods:

LCMS Method A: Waters Acquity UPLC system employing a Waters Acquity UPLC BEH C18, 1.7 μm, 50×2.1 mm column with an aqueous acetonitrile based solvent gradient of 2-98% CH₃CN/H₂O (0.05% TFA) over 9.5 mins. Flow rate=0.8 mL/min.

LCMS Method B: Waters Acquity UPLC system employing a Waters Acquity UPLC BEH C18, 1.7 μm, 50×2.1 mm column with an aqueous acetonitrile based solvent gradient of 2-98% CH₃CN/H₂O (0.05% TFA) over 1.0 mins. Flow rate=0.8 mL/min.

LCMS Method C: Shimadzu UFLC system employing an ACE UltraCore Super PhenylHexyl, 2.5 μm, 50×2.1 mm column with an aqueous acetonitrile based solvent gradient of 5-100% CH₃CN/H₂O (0.05% Formic acid) over 5.0 mins. Flow rate=1.0 mL/min.

LCMS Method D: Waters Acquity UPLC system employing a Waters Acquity UPLC C18, 1.7 μm, 50×2.1 mm column with an aqueous acetonitrile based solvent gradient of 5-95% CH₃CN/H₂O (0.05% Formic acid) over 4.0 mins. Flow rate=0.5 mL/min LCMS Method E: X Bridge BEH C18, 2.5 μm, 50×2.1 mm column with an aqueous acetonitrile based solvent gradient of 5-95% CH₃CN/(10 mM Ammonium Acetate in Water) over 4.0 mins. Flow rate=0.5 mL/min

As described herein, “Enantiomer I” or “Diastereomer I” refers to the first enantiomer or diastereomer eluded from the chiral column under the specific chiral analytical conditions detailed for examples provided elsewhere herein; and “Enantiomer II” or “Diastereomer II” refers to the second enantiomer or diastereomer eluded from the chiral column under the specific chiral analytical conditions detailed for examples provided elsewhere herein. Such nomenclature does not imply or impart any particular relative and/or absolute configuration for these compounds.

### Example 1: Compounds

### 2,3,4,5-Tetrahydrophenanthridine-1,6-dione (IVa)

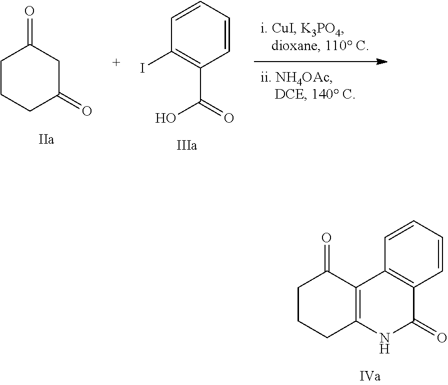 [SMILES: O=C1CCCC(=O)C1 ; O=C(O)c1ccccc1I ; O=C1CCCc2[nH]c(=O)c3ccccc3c21]

Step i: 2-Iodobenzoic acid (IIIa, 2.00 g, 8.06 mmol), cyclohexane-1,3-dione (IIa, 1.09 g, 9.68 mmol), copper (I) iodide (0.15 g, 0.81 mmol), and potassium phosphate (2.39 g, 11.29 mmol) were combined in dry 1,4-dioxane (12 mL) in a sealed tube under a nitrogen atmosphere. The mixture was stirred at room temperature for 30 min, and then at 110° C. for 3 hours. The reaction mixture was diluted with ethyl acetate (10 mL) and filtered through CELITE®, and the pad was washed with ethyl acetate (3×10 mL). The solvent was evaporated under high vacuum and the product was further purified by flash chromatography (Silicagel, ethyl acetate/hexane 0-90% gradient) to provide the intermediate 3,4-dihydro-2H-benzo[c]chromene-1,6-dione (1.09 g, 63%). ¹H NMR (400 MHz, CDCl₃): δ 9.05 (ddd, 1H), 8.28 (ddd, 1H), 7.83-7.74 (m, 1H), 7.52 (ddd, 1H), 2.94 (t, 2H), 2.70-2.62 (m, 2H), 2.17 (tt, 2H).

Step ii: 3,4-Dihydro-2H-benzo[c]chromene-1,6-dione (1.09 g, 5.11 mmol), obtained in Step i, and ammonium acetate (2.36 g, 30.67 mmol) were stirred in 1,2-dichloroethane (24 mL) at 140° C. in a sealed tube for 10 hours. The reaction mixture was allowed to cool to room temperature, diluted with dichloromethane (100 mL) and the organic phase was washed with saturated ammonium chloride (50 mL). The aqueous phase was extracted three times with dichloromethane (50 mL each) and the combined organic extracts were dried on sodium sulfate, then filtered. The solvent was evaporated, and the product was isolated by flash chromatography (Silicagel, methanol/dichloromethane 0-5% gradient), to provide 784 mg (72% yield) of 2,3,4,5-tetrahydrophenanthridine-1,6-dione (IVa). LCMS: m/z found 214.1 [M+H]⁺; ¹H NMR (400 MHz, CDCl₃): δ 11.32 (s, 1H), 9.32 (ddd, 1H), 8.41 (ddd, 1H), 7.83-7.74 (m, 1H), 7.52 (ddd, 1H), 3.05 (t, 2H), 2.70 (dd, 2H), 2.28-2.11 (m, 2H).

### 1-(Methylamino)-2,3,4,5-tetrahydro-1H-phenanthridin-6-one (Va)

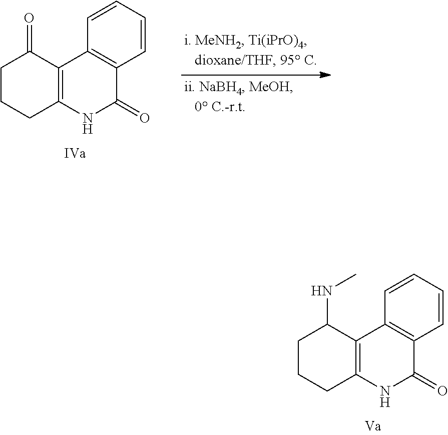 [SMILES: O=C1CCCc2[nH]c(=O)c3ccccc3c21 ; CNC1CCCc2[nH]c(=O)c3ccccc3c21]

Tetraisopropoxytitanium (0.28 mL, 0.94 mmol) was added to a mixture of 2,3,4,5-tetrahydrophenanthridine-1,6-dione (50 mg, 0.23 mmol) in 1,4-dioxane (1 mL) and a 2 M solution of methylamine in THE (1.17 mL, 2.34 mmol). The mixture then was heated at 95° C. under a nitrogen atmosphere in a sealed tube for 16 hours. The reaction mixture was diluted with 1 mL of anhydrous methanol and cooled in an ice bath. Sodium borohydride (17.75 mg, 0.47 mmol) was added in one portion and after 5 minutes the ice bath was removed. After an additional 20 min the reaction was quenched by addition of brine (1 mL), diluted with 20 mL of ethyl acetate, and stirred for an additional 15 min. The mixture was filtered through CELITE®, and the filter cake was washed with an additional 25 mL of ethyl acetate. The combined organic solution was dried over sodium sulfate, filtered and the solvent evaporated to provide 45 mg (84% yield) of 1-(methylamino)-2,3,4,5-tetrahydro-1H-phenanthridin-6-one (Va), which was used without further purification in the next step. LCMS: m/z found 216 [M−(MeNH₂)+H₂O+H]⁺; ¹H NMR (400 MHz, CDCl₃): δ 9.67-9.59 (m, 1H), 8.39 (ddd, 1H), 7.85-7.61 (m, 2H), 7.44 (ddd, 1H), 3.87 (d, 1H), 2.73-2.52 (m, 5H), 2.28-2.15 (m, 1H), 2.17-1.96 (m, 1H), 1.89-1.80 (m, 1H), 1.63-1.49 (m, 1H).

### 3-(3-Chloro-4-fluorophenyl)-1-methyl-1-(6-oxo-1,2,3,4,5,6-hexahydrophenanthridin-1-yl)urea (Compound 6/Compound 16/Compound 17)

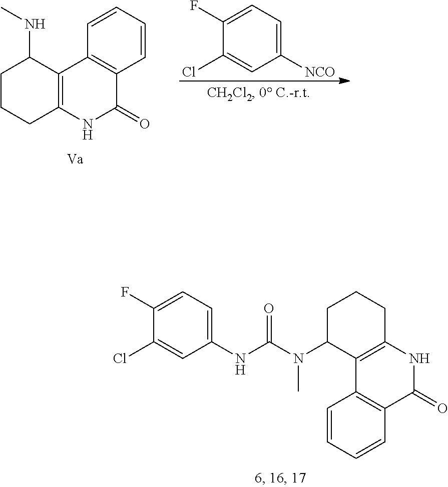

A solution of 2-chloro-1-fluoro-4-isocyanato-benzene (21 uL, 0.16 mmol) in 0.5 mL dichloromethane was added slowly to a stirred mixture of crude 1-(methylamino)-2,3,4,5-tetrahydro-1H-phenanthridin-6-one (Va, 45 mg, 0.20 mmol) in 1 mL of dichloromethane at 0° C. After 10 min, methanol (0.1 mL) was added and the reaction mixture was evaporated to one half of the initial volume, then directly loaded on a preconditioned Silicagel column. The product was purified by flash chromatography (Silicagel, ethyl acetate/hexane 20-100%) to provide racemic 3-(3-chloro-4-fluorophenyl)-1-methyl-1-(6-oxo-1,2,3,4,5,6-hexahydrophenanthridin-1-yl)urea (Compound 6, 38 mg, 48%). LCMS: m/z found 400.2/402.2 [M+H]⁺; RT=4.31 min, (Method A); ¹H NMR (400 MHz, DMSO-d6) δ 11.31 (s, 1H), 8.46 (s, 1H), 8.20 (dd, J=8.0, 1.4 Hz, 1H), 7.90 (dd, 1H), 7.70 (ddd, 1H), 7.53 (ddd, 1H), 7.48-7.37 (m, 2H), 7.32 (t, 1H), 5.59 (br. s, 1H), 2.73-2.60 (m, 4H), 2.57 (t, 1H), 1.89 (s, 1H), 1.95-1.69 (m, 1H).

The enantiomers were subsequently separated by SFC (Waters), Column: IG-semiprep (10 mm×250 mm) 5μ, 35% IPA in CO₂, Flow rate 9 ml/min to provide 9.5 mg and 7.2 mg of the resolved enantiomers.

3-(3-Chloro-4-fluorophenyl)-1-methyl-1-(6-oxo-1,2,3,4,5,6-hexahydrophenanthridin-1-yl)urea: Enantiomer I (Compound 16). LCMS: m/z found 400.1/402.1 [M+H]⁺; RT=4.38 min, (Method A); ¹H NMR (400 MHz, CDCl₃) δ 11.12 (s, 1H), 8.44 (dd, 1H), 7.74-7.61 (m, 2H), 7.54 (d, 1H), 7.51-7.42 (m, 1H), 7.31-7.22 (m, 1H), 7.08 (td, 1H), 6.41 (s, 1H), 5.81 (s, 1H), 2.92-2.74 (m, 2H), 2.73 (s, 3H), 2.13-2.02 (m, 2H), 1.91 (dt, 2H); Chiral analytical SFC: RT=6.23 min, Column: IG-analytical; 35% IPA in CO₂; Flow rate=3.0 g/min.

3-(3-Chloro-4-fluorophenyl)-1-methyl-1-(6-oxo-1,2,3,4,5,6-hexahydrophenanthridin-1-yl)urea: Enantiomer II (Compound 17). LCMS: m/z found 400.1/402.2 [M+H]⁺; RT=4.38 min, (Method A); ¹H NMR (400 MHz, CDCl₃) δ 11.02 (s, 1H), 8.44 (dd, 1H), 7.74-7.62 (m, 2H), 7.54 (dd, 1H), 7.47 (ddd, 1H), 7.31-7.22 (m, 1H), 7.08 (t, 1H), 6.40 (s, 1H), 5.81 (s, 1H), 2.91-2.74 (m, 2H), 2.73 (s, 3H), 2.13-2.02 (m, 2H), 1.91 (dt, 2H); Chiral analytical SFC: RT=11.47 min, Column: IG-analytical; 35% IPA in CO₂; Flow rate=3.0 g/min.

Compound 17 was also prepared independently as described in the procedures below, according to Scheme 2.

### 6-Methoxy-3,4-dihydro-2H1-phenanthridin-1-one (VII-A) and 5-methyl-3,4-dihydro-2H1-phenanthridine-1,6-dione (IV-B)

2,3,4,5-Tetrahydrophenanthridine-1,6-dione (IVa, 0.22 g, 1.03 mmol), iodomethane (321 uL, 5.16 mmol), and silver carbonate (0.17 g, 0.62 mmol) were stirred in chloroform (4 mL) at room temperature in a sealed tube for 48 hours. LCMS analysis indicated ˜60% conversion. The reaction mixture was heated at 50° C. for 2 h, when LCMS analysis indicated complete conversion, with no significant change in regioselectivity. The reaction mixture was cooled to room temperature, diluted with dichloromethane, and filtered through CELITE®. The solvent was evaporated under reduced pressure and the products were separated by flash chromatography (Silicagel, ethyl acetate/hexanes 0-30%) to provide: 6-methoxy-3,4-dihydro-2H-phenanthridin-1-one (VII-A, 192 mg, 82% yield); LCMS: m/z found 228.1 [M+H]⁺; RT=1.31 min, (Method B); ¹H NMR (400 MHz, CDCl₃) δ 9.37 (dt, 1H), 8.23 (ddq, 1H), 7.80-7.70 (m, 1H), 7.55-7.46 (m, 1H), 4.16 (s, 3H), 3.17-3.09 (m, 2H), 2.72 (ddd, 2H), 2.17 (pd, 2H); and 5-methyl-3,4-dihydro-2H-phenanthridine-1,6-dione (IV-B, 29 mg, 12%); LCMS: m/z found 228.2 [M+H]⁺; RT=0.96 min, (Method B); ¹H NMR (400 MHz, CDCl₃) δ 9.22 (dt, 1H), 8.42 (ddd, 1H), 7.72 (ddd, 1H), 7.49 (ddd, 1H), 3.69 (s, 3H), 3.03 (t, 2H), 2.69-2.61 (m, 2H), 2.26-2.14 (m, 2H).

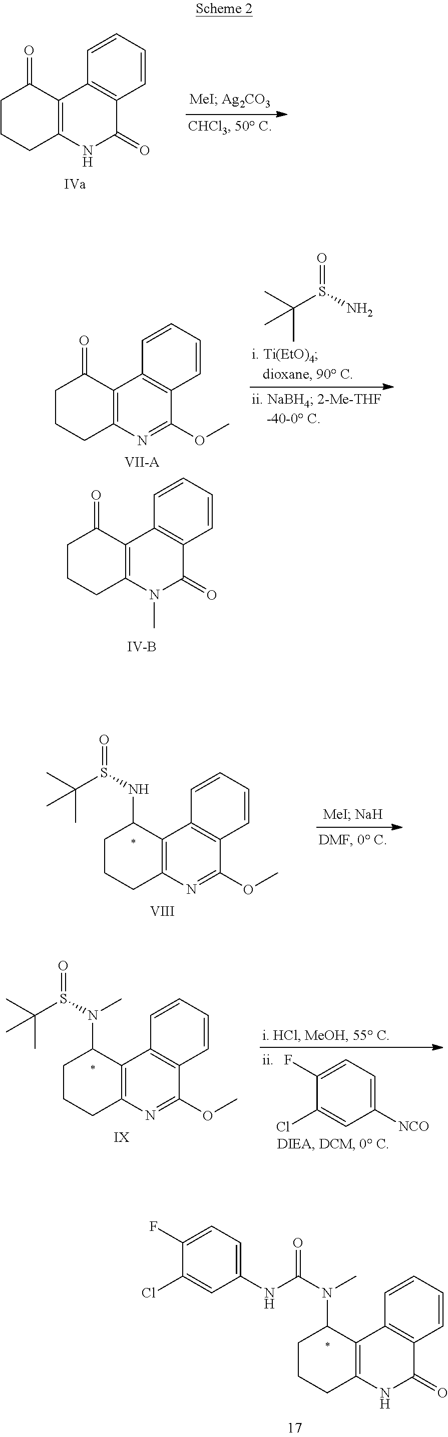

### (R)—N-(6-Methoxy-1,2,3,4-tetrahydrophenanthridin-1-yl)-2-methyl-propane-2-sulfinamide (VIII)

A mixture of (R)-2-methylpropane-2-sulfinamide (0.21 g, 1.76 mmol) and technical grade tetraethoxytitanium (0.28 mL, 1.32 mmol) were stirred in a sealed vial at 90° C. for 48 hours. A solution of 6-methoxy-3,4-dihydro-2H-phenanthridin-1-one (VII-A, 50.00 mg, 0.22 mmol) in anhydrous dioxane (0.2 mL) was then added and stirring was continued for 24 hours. Another portion of (R)-2-methylpropane-2-sulfinamide (0.21 g, 1.76 mmol) was added and stirring was continued for an additional 24 hours. The reaction mixture was allowed to cool to room temperature and diluted with 3 mL anhydrous 2-methyltetrahydrofuran, then cooled further to −40° C. Sodium borohydride (25 mg, 0.66 mmol) was added in one portion. The reaction temperature was maintained between −40 and −20° C. for 50 min, then at −10° C. for 1 hours. The reaction was then slowly warmed to 0° C. over 30 min. The reaction was quenched with 0.5 mL brine at 0° C. and was diluted with ethyl acetate (25 mL), filtered through CELITE®, and the filter cake was washed with ethyl acetate (3×20 mL). The solvent was evaporated and the product was isolated by flash chromatography (Silicagel, EtOAc/Hexane 0-25%) to provide (R)—N-(6-methoxy-1,2,3,4-tetrahydrophenanthridin-1-yl)-2-methyl-propane-2-sulfinamide (VIII, 18.7 mg, 26%). LCMS m/z found 333.3 [M+H]⁺; RT=4.45 min (Method B); ¹H NMR (400 MHz, CDCl₃) δ 8.27-8.15 (m, 2H), 7.75 (ddd, 1H), 7.49 (ddd, 1H), 5.07 (s, 1H), 4.10 (s, 3H), 3.29 (s, 1H), 3.00-2.79 (m, 2H), 2.31-2.21 (m, 1H), 2.14 (dddd, 1H), 1.91-1.69 (m, 1H), 1.45 (s, 9H), 1.37-1.21 (m, 1H).

### (R)—N-(6-methoxy-1,2,3,4-tetrahydrophenanthridin-1-yl)-N,2-dimethyl-propane-2-sulfinamide (IX)

A solution of (R)—N-(6-methoxy-1,2,3,4-tetrahydrophenanthridin-1-yl)-2-methyl-propane-2-sulfinamide (VIII, 18.0 mg, 0.05 mmol) in 1 mL anhydrous DMF, under nitrogen, was cooled in an ice bath and sodium hydride, 60% w/w in mineral oil (4.33 mg, 0.11 mmol) was added in one portion. After 20 minutes at 0° C., iodomethane (6.7 uL, 0.11 mmol) was added. The reaction was then stirred at 0° C. for 90 minutes. The reaction mixture was quenched by the slow addition of 1 mL of water, extracted with diethyl ether, and the combined organic extracts were washed 2× with water, then with brine, dried over sodium sulfate, decanted, and the solvent was evaporated under vacuum. The crude product was used in the next step without further purification: (R)—N-(6-methoxy-1,2,3,4-tetrahydrophenanthridin-1-yl)-N,2-dimethyl-propane-2-sulfinamide (IX, 16 mg, 85%). LCMS m/z found 347.3 [M+H]⁺; RT=4.49 min (Method B); ¹H NMR (400 MHz, CDCl₃) δ 8.21 (ddd, 1H), 7.90 (dt, 1H), 7.69 (ddd, 1H), 7.46 (ddd, 1H), 4.86 (dd, 1H), 4.11 (s, 3H), 3.13-2.99 (m, 1H), 2.86 (ddd, 1H), 2.58 (s, 3H), 2.35-2.19 (m, 1H), 2.00-1.80 (m, 1H), 1.63 (s, 1H), 1.01 (s, 8H), 0.92-0.80 (m, 1H).

### 3-(3-Chloro-4-fluorophenyl)-1-methyl-1-(6-oxo-1,2,3,4,5,6-hexahydrophenanthridin-1-yl)urea: Enantiomer II (Compound 17)

Step i: Hydrogen chloride 1.25 M in methanol (1.85 mL, 2.31 mmol) was added to (R)—N-(6-methoxy-1,2,3,4-tetrahydrophenanthridin-1-yl)-N,2-dimethyl-propane-2-sulfinamide (IX, 16.0 mg, 0.05 mmol) and the mixture was stirred at room temperature for 1 hour and then at 55° C. for 16 hours. The volatiles were removed in vacuo and the residue was azeotroped 2× with toluene, then further dried under high vacuum and used in the next reaction without further purification.

Step ii: The crude, enantioenriched 1-(methylamino)-2,3,4,5-tetrahydro-1H-phenanthridin-6-one (10.54 mg, 0.05 mmol) obtained in the previous step was suspended in 0.5 mL dichloromethane and treated at 0° C. with diisopropylethylamine (20 uL, 0.12 mmol). A solution of 2-chloro-1-fluoro-4-isocyanato-benzene (5.2 uL, 0.04 mmol) in 0.5 mL dichloromethane was added slowly and stirring was continued for 1 hour. The reaction was quenched with 0.5 mL MeOH, and after 5 min the mixture was adsorbed onto Silicagel and the product was isolated by flash-chromatography (Silicagel, ethyl acetate/hexanes 10-95%) and dried under high vacuum to provide 3-(3-chloro-4-fluorophenyl)-1-methyl-1-(6-oxo-1,2,3,4,5,6-hexahydrophenanthridin-1-yl)urea: Enantiomer II (Compound 17, 10.5 mg, 57%). LCMS: m/z found 400.1/402.2 [M+H]⁺; RT=4.38 min, (Method A); ¹H NMR (400 MHz, Methanol-d4) δ 8.32 (ddd, 1H), 7.76-7.66 (m, 2H), 7.58-7.44 (m, 2H), 7.39 (ddd, 1H), 7.17 (t, 1H), 5.72 (s, 1H), 2.83-2.60 (m, 2H), 2.69 (s, 3H), 2.14-1.81 (m, 4H); Chiral analytical SFC: RT=11.62 min, Column: IG-analytical; 35% IPA in CO₂; Flow rate=3.0 g/min.

### 3-(3-Chloro-4-fluorophenyl)-1-methyl-1-(5-methyl-6-oxo-1,2,3,4,5,6-hexahydrophenanthridin-1-yl)urea (Compound 31)

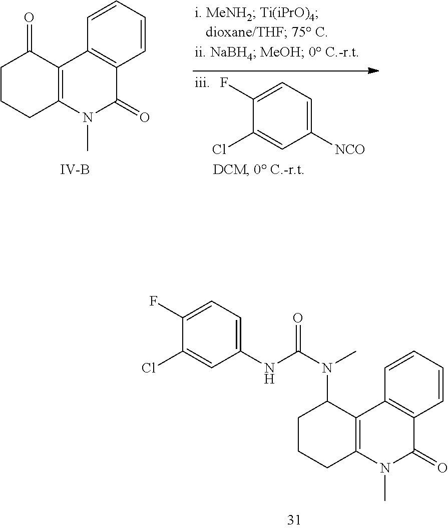

3-(3-Chloro-4-fluorophenyl)-1-methyl-1-(5-methyl-6-oxo-1,2,3,4,5,6-hexahydrophenanthridin-1-yl)urea was synthesized in an analogous manner as described above for Compound 6, from 5-methyl-3,4-dihydro-2H-phenanthridine-1,6-dione (IV-B). LCMS: m/z found 414.3/416.3 [M+H]⁺; RT=4.59 min, (Method A); ¹H NMR (400 MHz, DMSO-d6) δ 8.46 (s, 1H), 8.27 (ddd, 1H), 7.91 (dd, 1H), 7.71 (ddd, 1H), 7.54 (ddd, 1H), 7.51-7.39 (m, 2H), 7.32 (t, 1H), 5.64 (s, 1H), 3.56 (s, 3H), 2.91 (d, 1H), 2.81-2.70 (m, 1H), 2.63 (s, 3H), 1.87 (m, 4H).

### 3-(3-Chloro-4-fluorophenyl)-1-(6-methoxy-1,2,3,4-tetrahydrophenanthridin-1-yl)-1-methylurea (Compound 34)

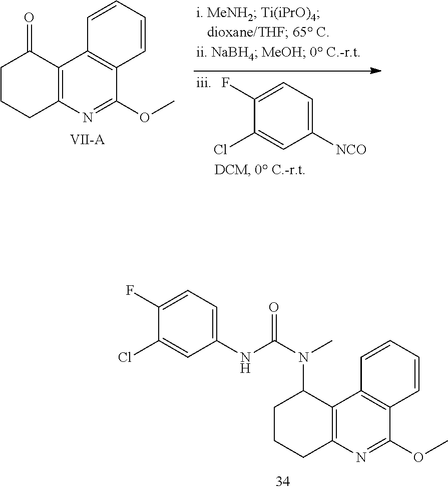

3-(3-Chloro-4-fluorophenyl)-1-(6-methoxy-1,2,3,4-tetrahydrophenanthridin-1-yl)-1-methylurea was synthesized in an analogous manner as described above for Compound 6, from 6-methoxy-3,4-dihydro-2H-phenanthridin-1-one (VII-A). LCMS: m/z found 414.3/416.3 [M+H]⁺; RT=5.60 min, (Method A); ¹H NMR (400 MHz, DMSO-d6) δ 8.49 (s, 1H), 8.22-8.14 (m, 1H), 7.91 (dd, 1H), 7.76 (ddd, 1H), 7.70-7.63 (m, 1H), 7.60-7.50 (m, 2H), 7.32 (t, 1H), 5.87 (s, 1H), 4.06 (s, 3H), 3.04-2.93 (m, 1H), 2.81 (dt, 1H), 2.51 (s, 3H), 2.05-1.90 (m, 3H), 1.85-1.69 (m, 1H).

### 1-(3-Hydroxypropylamino)-2,3,4,5-tetrahydro-1H-phenanthridin-6-one (Vb)

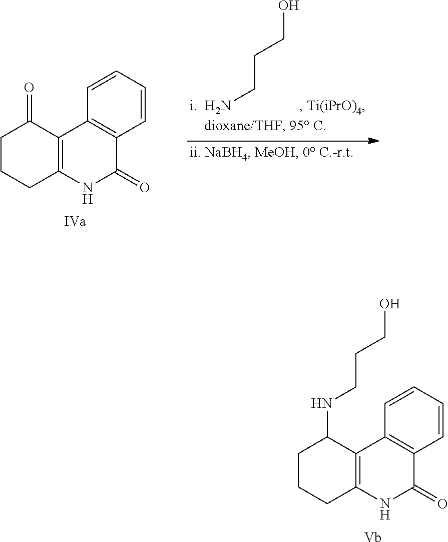

1-(3-Hydroxypropylamino)-2,3,4,5-tetrahydro-1H-phenanthridin-6-one was synthesized in an analogous manner as described above from 2,3,4,5-tetrahydrophenanthridine-1,6-dione (IVa) and 3-aminopropan-1-ol. ¹H NMR (400 MHz, CDCl₃) δ 8.46-8.36 (m, 1H), 7.70 (ddd, 1H), 7.59 (d, 1H), 7.44 (ddd, 1H), 3.95 (t, 1H), 2.71-2.58 (m, 2H), 2.27-2.17 (m, 1H), 2.08-1.91 (m, 1H), 1.87 (td, 1H), 1.74 (tt, 2H), 1.70-1.64 (m, 3H), 1.59 (tt, 1H), 1.29-1.20 (m, 1H).

### 3-(3-Chloro-4-fluorophenyl)-1-(3-hydroxypropyl)-1-(6-oxo-1,2,3,4,5,6-hexahydrophenanthridin-1-yl)urea (Compound 7)

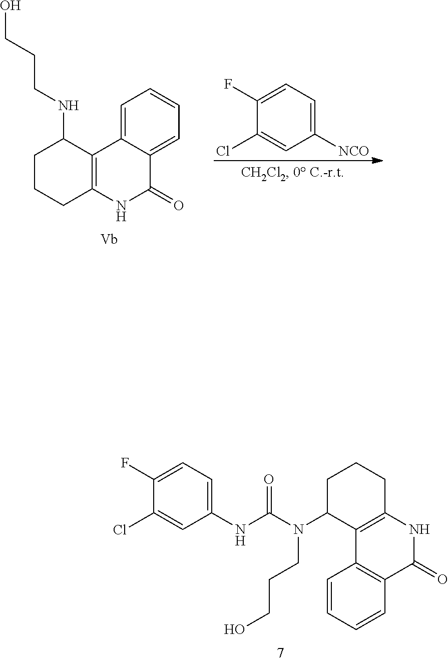

3-(3-Chloro-4-fluorophenyl)-1-(3-hydroxypropyl)-1-(6-oxo-1,2,3,4,5,6-hexahydrophenanthridin-1-yl)urea was synthesized in an analogous manner as described above from racemic 1-(3-hydroxypropylamino)-2,3,4,5-tetrahydro-1H-phenanthridin-6-one (Vb) and 2-chloro-1-fluoro-4-isocyanato-benzene. LCMS: m/z found 444.1/446.2 [M+H]⁺; RT=4.21 min, (Method A); ¹H NMR (400 MHz, DMSO-d₆) δ 11.30 (s, 1H), 8.68 (s, 1H), 8.19 (dd, 1H), 7.85 (dd, 1H), 7.69 (ddd, 1H), 7.51-7.38 (m, 3H), 7.33 (t, 1H), 5.62 (s, 1H), 4.94 (s, 1H), 3.15 (tt, 4H), 2.68 (m, 1H), 2.56 (q, 1H), 2.01-1.82 (m, 3H), 1.74 (s, 1H), 1.31-1.22 (m, 1H), 1.14 (s, 1H).

### 1-(Isobutylamino)-2,3,4,5-tetrahydro-1H-phenanthridin-6-one (Vc)

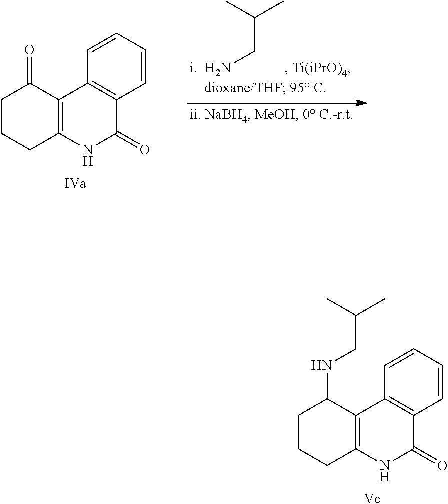

1-(Isobutylamino)-2,3,4,5-tetrahydro-1H-phenanthridin-6-one was synthesized in an analogous manner as described above from 2,3,4,5-tetrahydrophenanthridine-1,6-dione (IVa) and 2-methylpropan-1-amine. LCMS: m/z found 198.1 [M−(Me₂CHCH₂NH)]⁺; RT=0.66 min, (Method B); ¹H NMR (400 MHz, CDCl₃) δ 10.45 (s, 1H), 8.41 (dt, 1H), 7.76-7.63 (m, 2H), 7.52-7.39 (m, 1H), 3.93 (t, 1H), 2.71-2.60 (m, 3H), 2.54 (dd, 1H), 2.23-2.13 (m, 1H), 2.08 (tdd, 1H), 1.80 (ddd, 1H), 1.70 (dp, 1H), 1.54 (tt, 1H), 1.25 (s, 1H), 1.08-0.79 (m, 6H).

### 3-(3-Chloro-4-fluorophenyl)-1-isobutyl-1-(6-oxo-1,2,3,4,5,6-hexahydrophenanthridin-1-yl)urea (Compound 8)

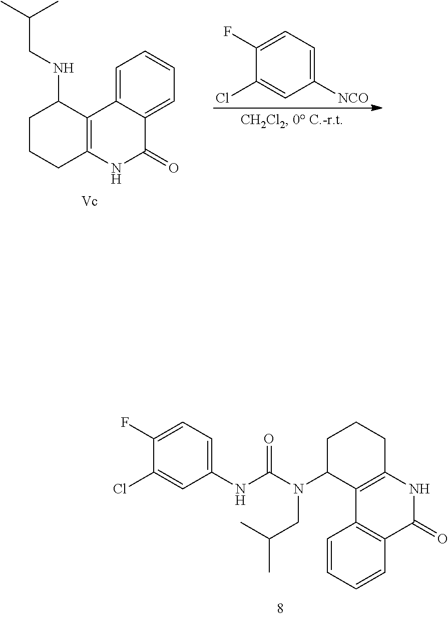

3-(3-Chloro-4-fluorophenyl)-1-isobutyl-1-(6-oxo-1,2,3,4,5,6-hexahydrophenanthridin-1-yl)urea was synthesized in an analogous manner as described above from racemic 1-(isobutylamino)-2,3,4,5-tetrahydro-1H-phenanthridin-6-one (Vc) and 2-chloro-1-fluoro-4-isocyanato-benzene. LCMS: m/z found 442.2/444.3 [M+H]⁺; RT=1.08 min, (Method B); ¹H NMR (400 MHz, DMSO-d₆) δ 11.26 (s, 1H), 8.49 (s, 1H), 8.17 (dd, 1H), 7.81 (dd, 1H), 7.69 (ddd, 1H), 7.55 (d, 1H), 7.48 (ddd, 1H), 7.46-7.37 (m, 1H), 7.30 (t, 1H), 5.62 (s, 1H), 3.00 (dd, 1H), 2.83 (dd, 1H), 2.72-2.65 (m, 1H), 2.54 (q, 1H), 2.00 (m, 1H), 1.89-1.80 (m, 2H), 1.70 (m, 1H), 1.34 (dt, 1H), 0.61 (d, J=6.7 Hz, 3H), 0.43 (d, 3H).

### 8-Fluoro-2,3,4,5-tetrahydrophenanthridine-1,6-dione (IVb)

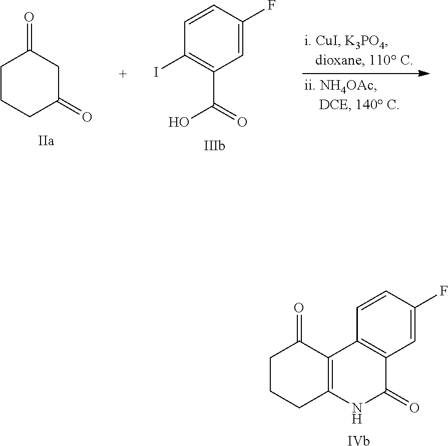

Step i: 5-Fluoro-2-iodo-benzoic acid (IIIb, 543.7 mg, 2.04 mmol), cyclohexane-1,3-dione (IIa, 275.02 mg, 2.45 mmol), copper (I) iodide (38.93 mg, 0.20 mmol), and potassium phosphate (606.64 mg, 2.86 mmol) were combined in a tube under a nitrogen atmosphere. Anhydrous 1,4-dioxane (1.5 mL) was added and the reaction tube was purged with nitrogen and stirred at room temperature for 30 min, and then heated at 110° C. for 4 hours. The reaction mixture was allowed to cool to room temperature, diluted with ethyl acetate (10 mL), filtered through CELITE®, and the pad was washed with ethyl acetate (3×25 mL). The combined organic extracts were dried over sodium sulfate, filtered and the solvent was evaporated under high vacuum to afford 434 mg (91% yield) of 8-fluoro-3,4-dihydro-2H-benzo[c]chromene-1,6-dione of satisfactory purity. ¹H NMR (400 MHz, CDCl₃) δ 9.11 (ddd, 1H), 7.91 (ddd, 1H), 7.49 (ddd, 1H), 2.93 (t, 2H), 2.69-2.58 (m, 2H), 2.23-2.11 (m, 2H).

Step ii: 8-fluoro-3,4-dihydro-2H-benzo[c]chromene-1,6-dione (434.00 mg, 1.87 mmol) from Step i and ammonium acetate (1.44 g, 18.69 mmol) were stirred in 1,2-dichloroethane (2 mL) at 140° C. in sealed tube for 10 h. The reaction mixture was cooled and diluted with dichloromethane and washed with saturated ammonium chloride. The aqueous phase was extracted three times with dichloromethane and the combined organic extracts were dried over sodium sulfate, then filtered. The solvent was evaporated, and the product was isolated by flash chromatography (Silicagel, methanol/dichloromethane 0-5% gradient), to provide 225 mg (52% yield) of 8-fluoro-2,3,4,5-tetrahydrophenanthridine-1,6-dione (IVb). LCMS: m/z found 232.1 [M+H]⁺; RT=0.82 min, (Method B); ¹H NMR (400 MHz, CDCl₃) δ 9.21 (dd, 1H), 7.87 (ddd, 1H), 7.38 (dddd, 1H), 2.83 (t, 2H), 2.57 (dd, 2H), 2.09 (dt, 2H).

### 8-Fluoro-1-(methylamino)-2,3,4,5-tetrahydro-1H-phenanthridin-6-one (Vd)

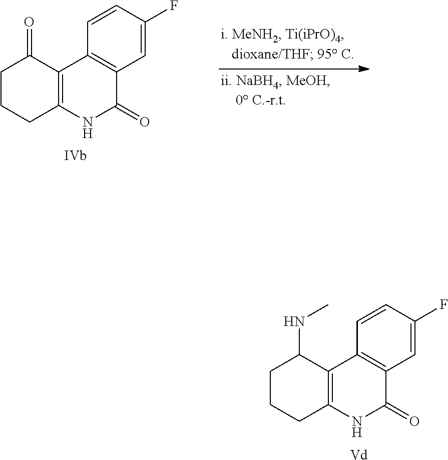

8-Fluoro-1-(methylamino)-2,3,4,5-tetrahydro-1H-phenanthridin-6-one was synthesized in an analogous manner as described above from 8-fluoro-2,3,4,5-tetrahydrophenanthridine-1,6-dione (IVb) and methanamine. LCMS: m/z found 216.1 [M−MeNH)]⁺; RT=0.60 min, (Method B); ¹H NMR (400 MHz, CDCl₃) δ 10.49 (s, 1H), 8.12-7.97 (m, 1H), 7.69 (dd, 1H), 7.43 (ddd, 1H), 3.86-3.82 (m, 1H), 2.65 (dd, 1H), 2.58 (s, 3H), 2.24 (dd, 1H), 2.08-1.91 (m, 1H), 1.85 (dt, 1H), 1.56 (tt, 1H), 1.25 (m, 1H).

### 3-(3-Chloro-4-fluorophenyl)-1-(8-fluoro-6-oxo-1,2,3,4,5,6-hexahydrophenanthridin-1-yl)-1-methylurea (Compound 9)

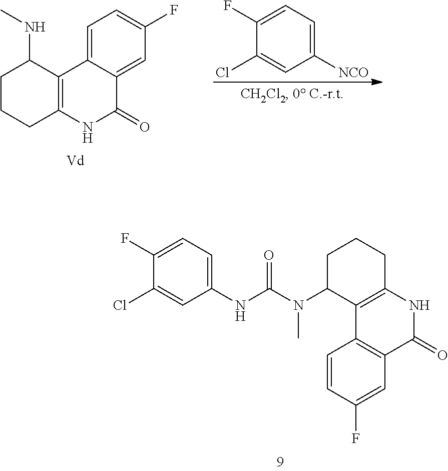

3-(3-Chloro-4-fluorophenyl)-1-(8-fluoro-6-oxo-1,2,3,4,5,6-hexahydrophenanthridin-1-yl)-1-methylurea was synthesized in an analogous manner as described above from racemic 8-fluoro-1-(methylamino)-2,3,4,5-tetrahydro-1H-phenanthridin-6-one (Vd) and 2-chloro-1-fluoro-4-isocyanato-benzene. LCMS: m/z found 418.1/420.1 [M+H]⁺; RT=4.56 min, (Method A); ¹H NMR (400 MHz, DMSO-d₆) δ 11.45 (s, 1H), 8.47 (s, 1H), 8.02-7.78 (m, 2H), 7.65 (td, 1H), 7.59-7.40 (m, 2H), 7.32 (t, 1H), 5.59 (s, 1H), 2.74-2.52 (m, 2H), 2.62 (s, 3H), 1.98-1.65 (m, 4H).

### 3-(3,4-Difluorophenyl)-1-(8-fluoro-6-oxo-1,2,3,4,5,6-hexahydrophenanthridin-1-yl)-1-methylurea (Compound 32)

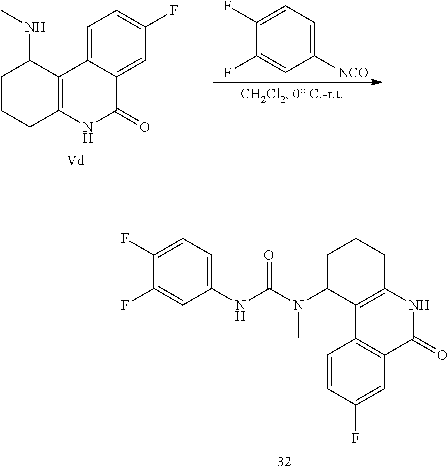

3-(3,4-Difluorophenyl)-1-(8-fluoro-6-oxo-1,2,3,4,5,6-hexahydrophenanthridin-1-yl)-1-methylurea was synthesized in an analogous manner as described above from racemic 8-fluoro-1-(methylamino)-2,3,4,5-tetrahydro-1H-phenanthridin-6-one (Vd) and 1,2-difluoro-4-isocyanato-benzene. LCMS: m/z found 402.3 [M+H]⁺; RT=4.23 min, (Method A); ¹H NMR (400 MHz, DMSO-d₆) δ 11.45 (s, 1H), 8.48 (s, 1H), 7.85 (dd, 1H), 7.77 (ddd, 1H), 7.65 (td, 1H), 7.47 (dd, 1H), 7.40-7.26 (m, 2H), 5.59 (s, 1H), 2.67 (dd, 1H), 2.62 (s, 3H), 2.56 (t, 1H), 1.97-1.61 (m, 4H).

### 1-(8-Fluoro-6-oxo-1,2,3,4,5,6-hexahydrophenanthridin-1-yl)-1-methyl-3-(3,4,5-trifluorophenyl)urea (Compound 33)

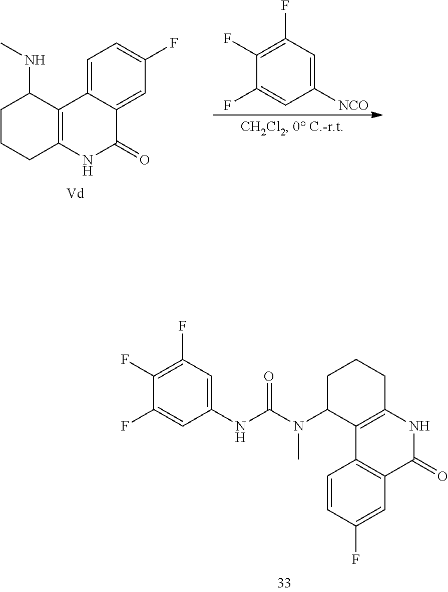

1-(8-Fluoro-6-oxo-1,2,3,4,5,6-hexahydrophenanthridin-1-yl)-1-methyl-3-(3,4,5-trifluorophenyl)urea was synthesized in an analogous manner as described above from racemic 8-fluoro-1-(methylamino)-2,3,4,5-tetrahydro-1H-phenanthridin-6-one (Vd) and 1,2,3-trifluoro-5-isocyanato-benzene. LCMS: m/z found 420.2 [M+H]⁺; RT=4.58 min, (Method A); ¹H NMR (400 MHz, DMSO-d₆) δ 11.46 (s, 1H), 8.62 (s, 1H), 7.85 (dd, 1H), 7.70-7.56 (m, 2H), 7.60-7.51 (m, 1H), 7.44 (dd, 1H), 5.58 (s, 1H), 2.67 (dd, 1H), 2.62 (s, 3H), 2.56 (t, 1H), 2.05-1.57 (m, 4H).

### 8-Fluoro-1-(isobutylamino)-2,3,4,5-tetrahydro-1H-phenanthridin-6-one (Ve)

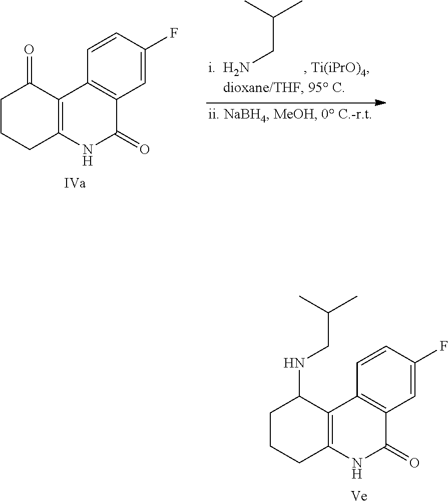

8-Fluoro-1-(isobutylamino)-2,3,4,5-tetrahydro-1H-phenanthridin-6-one was synthesized in an analogous manner as described above from 8-fluoro-2,3,4,5-tetrahydrophenanthridine-1,6-dione (IVb) and 2-methylpropan-1-amine. The product was purified by flash-chromatography (Silicagel, MeOH/DCM 0-10%). LCMS: m/z found 216.1 [M−(Me₂CHCH₂NH)]⁺; RT=0.70 min, (Method B); ¹H NMR (400 MHz, CDCl₃) δ 10.86 (s, 1H), 8.04 (dd, 1H), 7.73 (dd, 1H), 7.41 (ddd, 1H), 3.90 (t, 1H), 2.72-2.58 (m, 3H), 2.50 (dd, 1H), 2.25-2.14 (m, 1H), 2.08-1.94 (m, 1H), 1.83 (dt, 1H), 1.70 (dq, 1H), 1.54 (tt, 1H), 0.94 (dd, 6H).

### 8-Fluoro-1-(3-hydroxypropylamino)-2,3,4,5-tetrahydro-1H-phenanthridin-6-one (Vf)

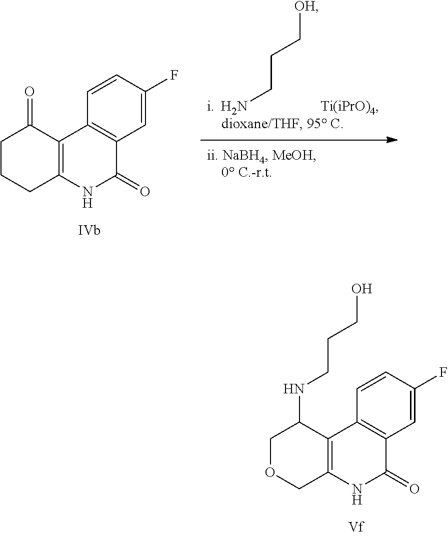 [SMILES: O=C1CCCc2[nH]c(=O)c3cc(F)ccc3c21 ; NCCCO ; O=c1[nH]c2c(c3ccc(F)cc13)C(NCCCO)COC2]

8-Fluoro-1-(3-hydroxypropylamino)-2,3,4,5-tetrahydro-1H-phenanthridin-6-one was synthesized in an analogous manner as described above from 8-fluoro-2,3,4,5-tetrahydrophenanthridine-1,6-dione (IVb) and 3-aminopropan-1-ol. LCMS: m/z found 216.1 [M−(HO(CH₂)₃NH)]⁺; RT=0.62 min, (Method B); ¹H NMR (400 MHz, CDCl₃) δ 10.96 (s, 1H), 8.03 (dd, 1H), 7.63 (dd, 1H), 7.43 (dddd, 1H), 3.94 (d, 1H), 3.82 (td, 2H), 3.11 (dt, 1H), 3.01 (dt, 1H), 2.89 (s, 1H), 2.66 (dd, 2H), 2.28-2.18 (m, 1H), 2.07-1.83 (m, 1H), 1.75 (hept, 2H), 1.66-1.53 (m, 1H).

### 3-(3-Chloro-4-fluorophenyl)-1-(8-fluoro-6-oxo-1,2,3,4,5,6-hexahydrophenanthridin-1-yl)-1-isobutylurea (Compound 10)

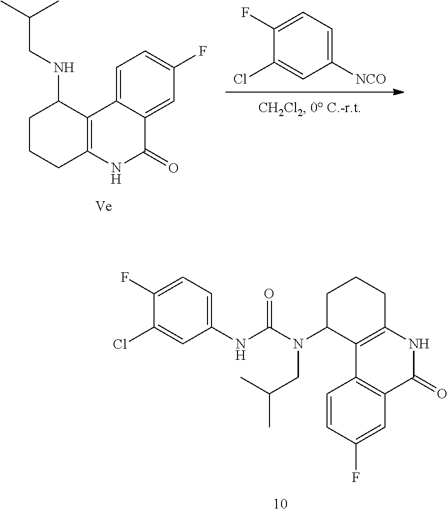

3-(3-Chloro-4-fluorophenyl)-1-(8-fluoro-6-oxo-1,2,3,4,5,6-hexahydrophenanthridin-1-yl)-1-isobutylurea was synthesized in an analogous manner as described above from racemic 8-fluoro-1-(isobutylamino)-2,3,4,5-tetrahydro-1H-phenanthridin-6-one (Ve) and 2-chloro-1-fluoro-4-isocyanato-benzene. LCMS: m/z found 460.2/462.2 [M+H]⁺; RT=5.28 min, (Method A); ¹H NMR (400 MHz, DMSO-d₆) δ 11.43 (s, 1H), 8.52 (s, 1H), 7.84 (ddd, 2H), 7.66 (dd, 2H), 7.49 (ddd, 1H), 7.32 (t, 1H), 5.65 (s, 1H), 3.05 (dd, 1H), 2.82 (dd, 1H), 2.69 (dt, 1H), 2.57 (t, 1H), 2.04-1.95 (m, 1H), 1.86 (m, 2H), 1.72 (m, 1H), 1.32 (dq, 1H), 0.63 (d, 3H), 0.44 (d, 3H).

### 3-(3-Chloro-4-fluorophenyl)-1-(8-fluoro-6-oxo-1,2,3,4,5,6-hexahydrophenanthridin-1-yl)-1-(3-hydroxypropyl)urea (Compound 11)

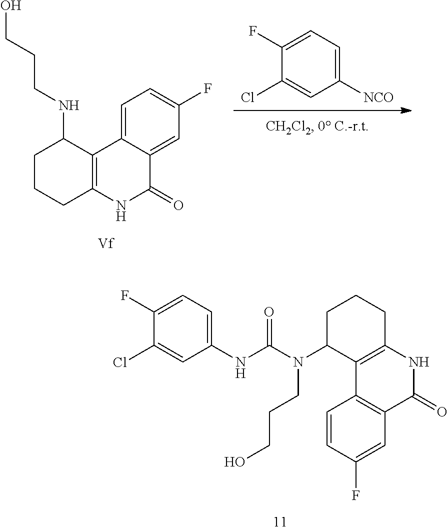

3-(3-Chloro-4-fluorophenyl)-1-(8-fluoro-6-oxo-1,2,3,4,5,6-hexahydrophenanthridin-1-yl)-1-(3-hydroxypropyl)urea was synthesized in an analogous manner as described above from racemic 8-fluoro-1-(3-hydroxypropylamino)-2,3,4,5-tetrahydro-1H-phenanthridin-6-one (Vf) and 2-chloro-1-fluoro-4-isocyanato-benzene. LCMS: m/z found 462.2 [M+H]⁺; RT=4.43 min, (Method A); ¹H NMR (400 MHz, DMSO-d₆) δ 11.45 (s, 1H), 8.68 (s, 1H), 7.85 (ddd, 2H), 7.65 (td, 1H), 7.58-7.42 (m, 2H), 7.33 (td, 1H), 5.62 (s, 1H), 4.94 (s, 1H), 3.24-2.96 (m, 4H), 2.80-2.62 (m, 1H), 2.57 (t, 1H), 2.05-1.79 (m, 3H), 1.74 (s, 1H), 1.25 (d, 1H), 1.11 (s, 1H).

### 3,4-Dihydro-2H-cyclopenta[c]isoquinoline-1,5-dione (IVc)

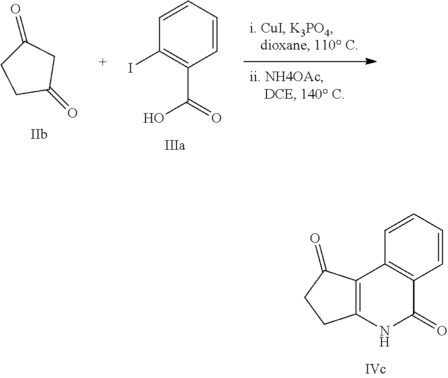 [SMILES: O=C1CCC(=O)C1 ; O=C(O)c1ccccc1I ; O=C1CCc2[nH]c(=O)c3ccccc3c21]

Step i: 2,3-Dihydrocyclopenta[c]isochromene-1,5-dione was synthesized in an analogous manner as described above from 2-iodobenzoic acid (IIIa) and cyclopentane-1,3-dione (IIb). ¹H NMR (400 MHz, CDCl₃): δ 8.51 (ddd, 1H), 8.29 (ddd, 1H), 7.82 (ddd, 1H), 7.58 (ddd, 1H), 3.08-3.00 (m, 2H), 2.80-2.72 (m, 2H).

Step ii: 3,4-Dihydro-2H-cyclopenta[c]isoquinoline-1,5-dione was synthesized in an analogous manner as described above from 2,3-dihydrocyclopenta[c]isochromene-1,5-dione and ammonium acetate. LCMS: m/z found 200.1 [M+H]⁺; RT=0.75 min (Method B); ¹H NMR (400 MHz, DMSO-d₆) δ 12.37 (s, 1H), 8.56 (ddd, 1H), 8.19 (ddd, 1H), 7.88-7.75 (m, 1H), 7.54 (ddd, 1H), 2.98-2.91 (m, 2H), 2.64-2.54 (m, 2H).

### 1-(Methylamino)-1,2,3,4-tetrahydrocyclopenta[c]isoquinolin-5-one (Vg)

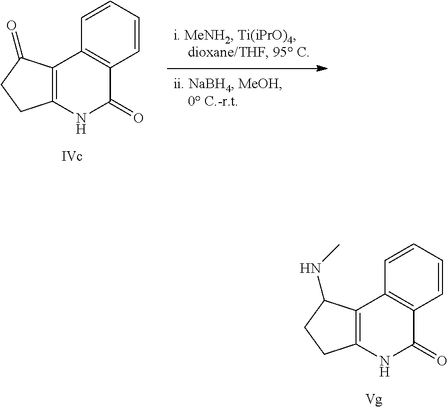 [SMILES: O=C1CCc2[nH]c(=O)c3ccccc3c21 ; CNC1CCc2[nH]c(=O)c3ccccc3c21]

1-(Methylamino)-1,2,3,4-tetrahydrocyclopenta[c]isoquinolin-5-one was synthesized in an analogous manner as described above from 3,4-dihydro-2H-cyclopenta[c]isoquinoline-1,5-dione (IVc) and methanamine. LCMS: m/z found 184.1 [M−MeNH)]⁺; RT=0.58 min, (Method B); ¹H NMR (400 MHz, CDCl₃) δ 11.17 (s, 1H), 8.38 (ddd, 1H), 7.76 (dt, 1H), 7.66 (ddd, 1H), 7.41 (ddd, 1H), 4.48 (dt, 1H), 3.22-3.04 (m, 1H), 2.83 (ddd, 1H), 2.47 (s, 3H), 2.39 (ddt, 1H), 2.13 (ddt, 1H).

### 1-(Isobutylamino)-1,2,3,4-tetrahydrocyclopenta[c]isoquinolin-5-one (Vh)

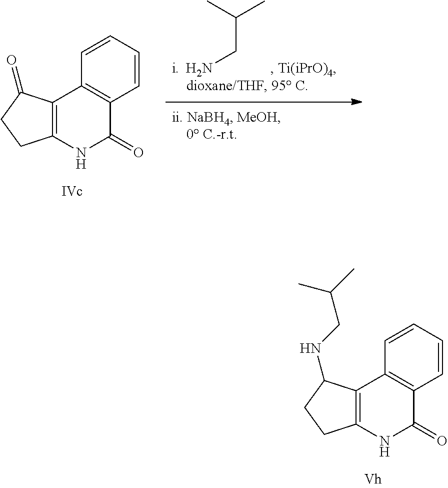

1-(Isobutylamino)-1,2,3,4-tetrahydrocyclopenta[c]isoquinolin-5-one was synthesized in an analogous manner as described above from 3,4-dihydro-2H-cyclopenta[c]isoquinoline-1,5-dione (IVc) and 2-methylpropan-1-amine. LCMS: m/z found 184.1 [M−(Me₂CHCH₂NH)]⁺; RT=0.69 min, (Method B); ¹H NMR (400 MHz, CDCl₃) δ 11.41 (s, 1H), 8.40 (dp, 1H), 7.82 (dt, 1H), 7.77-7.61 (m, 1H), 7.46-7.37 (m, 1H), 4.52-4.44 (m, 1H), 3.16-3.02 (m, 1H), 2.82 (ddd, 1H), 2.58-2.41 (m, 2H), 2.45-2.32 (m, 1H), 2.11-1.97 (m, 2H), 1.79-1.61 (m, 1H), 0.91 (ddd, 6H).

### 1-(3-Hydroxypropylamino)-1,2,3,4-tetrahydrocyclopenta[c]isoquinolin-5-one (Vi)

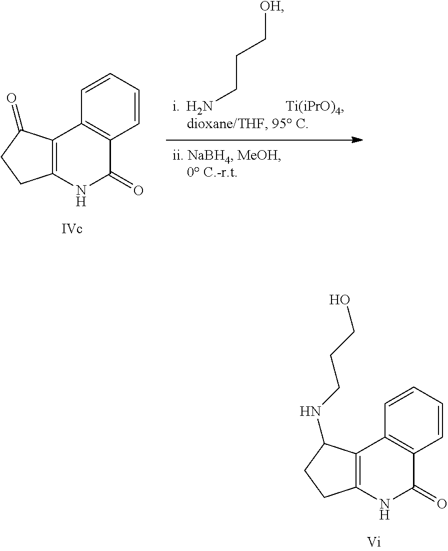 [SMILES: O=C1CCc2[nH]c(=O)c3ccccc3c21 ; NCCCO ; O=c1[nH]c2c(c3ccccc13)C(NCCCO)CC2]

1-(3-Hydroxypropylamino)-1,2,3,4-tetrahydrocyclopenta[c]isoquinolin-5-one was synthesized in an analogous manner as described above from 3,4-dihydro-2H-cyclopenta[c]isoquinoline-1,5-dione (IVc) and 3-aminopropan-1-ol. LCMS: m/z found 184.1 [M−(HO(CH₂)₃NH)]⁺; RT=0.59 min, (Method B); ¹H NMR (400 MHz, CDCl₃) δ 8.43-8.34 (m, 1H), 7.73-7.57 (m, 2H), 7.42 (ddd, 1H), 4.49 (dt, 1H), 3.88-3.70 (m, 2H), 3.08 (dddd, 1H), 2.97 (ddd, 1H), 2.90 (ddd, 1H), 2.82 (ddd, 1H), 2.36 (ddt, 1H), 2.21-2.06 (m, 1H), 1.82-1.61 (m, 2H).

### 3-(3-Chloro-4-fluorophenyl)-1-methyl-1-(5-oxo-2,3,4,5-tetrahydro-1H-cyclopenta[c]isoquinolin-1-yl)urea (Compound 12)

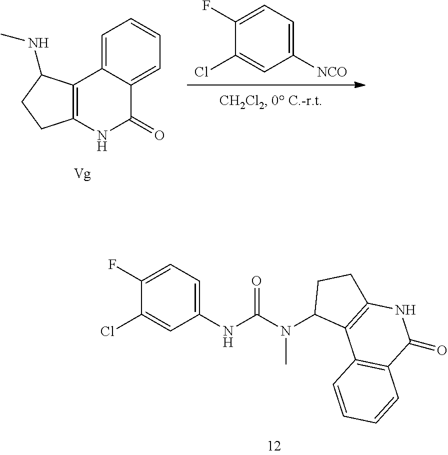

3-(3-Chloro-4-fluorophenyl)-1-methyl-1-(5-oxo-2,3,4,5-tetrahydro-1H-cyclopenta[c]isoquinolin-1-yl)urea was synthesized in an analogous manner as described above from racemic 1-(methylamino)-1,2,3,4-tetrahydrocyclopenta[c]isoquinolin-5-one (Vg) and 2-chloro-1-fluoro-4-isocyanato-benzene. LCMS: m/z found 386.2/388.2 [M+H]⁺; RT=4.23 min, (Method A); ¹H NMR (400 MHz, DMSO-d₆) δ 11.64 (s, 1H), 8.56 (s, 1H), 8.19 (dq, 1H), 7.87 (ddd, 1H), 7.73-7.64 (m, 1H), 7.52 (dddd, 1H), 7.47-7.38 (m, 2H), 7.32 (td, 1H), 6.06 (d, 1H), 2.98 (dt, 1H), 2.73 (ddd, 1H), 2.59 (s, 3H), 2.56-2.43 (m, 1H), 1.96-1.83 (m, 1H).

### 3-(3-Chloro-4-fluorophenyl)-1-isobutyl-1-(5-oxo-2,3,4,5-tetrahydro-1H-cyclopenta[c]isoquinolin-1-yl)urea (Compound 13)

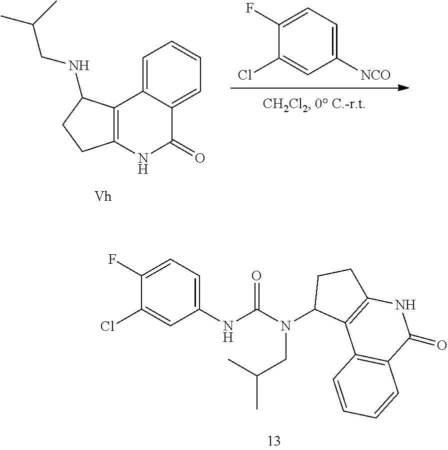

3-(3-Chloro-4-fluorophenyl)-1-isobutyl-1-(5-oxo-2,3,4,5-tetrahydro-1H-cyclopenta[c]isoquinolin-1-yl)urea urea was synthesized in an analogous manner as described above from racemic 1-(isobutylamino)-1,2,3,4-tetrahydrocyclopenta[c]isoquinolin-5-one (Vh) and 2-chloro-1-fluoro-4-isocyanato-benzene. LCMS: m/z found 428.1/430.3 [M+H]⁺; RT=1.05 min, (Method B); ¹H NMR (400 MHz, DMSO-d₆) δ 11.61 (s, 1H), 8.54 (s, 1H), 8.18 (ddd, 1H), 7.78 (dd, 1H), 7.70 (ddd, 1H), 7.56-7.49 (m, 1H), 7.49-7.37 (m, 2H), 7.30 (t, 1H), 5.85 (s, 1H), 3.10-2.84 (m, 3H), 2.72 (ddd, 1H), 2.66-2.53 (m, 1H), 1.99 (s, 1H), 1.50 (s, 1H), 0.64 (dd, 6H).

### 3-(3-Chloro-4-fluorophenyl)-1-(3-hydroxypropyl)-1-(5-oxo-2,3,4,5-tetrahydro-1H-cyclopenta[c]isoquinolin-1-yl)urea (Compound 14)

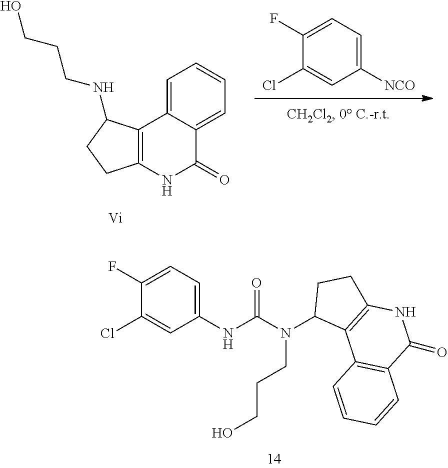

3-(3-Chloro-4-fluorophenyl)-1-(3-hydroxypropyl)-1-(5-oxo-2,3,4,5-tetrahydro-1H-cyclopenta[c]isoquinolin-1-yl)urea was synthesized in an analogous manner as described above from racemic 1-(3-hydroxypropylamino)-1,2,3,4-tetrahydrocyclopenta[c]isoquinolin-5-one (Vi) and 2-chloro-1-fluoro-4-isocyanato-benzene. LCMS: m/z found 430.2 [M+H]⁺; RT=4.11 min, (Method A); ¹H NMR (400 MHz, DMSO-d₆) δ 11.64 (s, 1H), 8.73 (s, 1H), 8.18 (dd, 1H), 7.81 (dd, 1H), 7.73-7.64 (m, 1H), 7.49-7.37 (m, 3H), 7.32 (t, 1H), 6.04 (d, 1H), 4.92 (t, 1H), 3.23 (d, 1H), 3.24-3.10 (m, 1H), 3.06 (td, 2H), 2.73 (ddd, 1H), 2.57 (dt, 1H), 2.01-1.86 (m, 1H), 1.32 (d, 2H).

### 8,9-Difluoro-2,3,4,5-tetrahydrophenanthridine-1,6-dione (IVd)

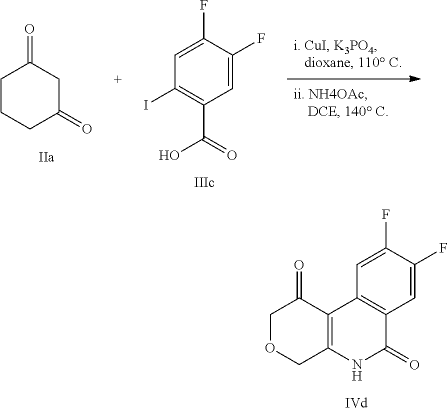 [SMILES: O=C1CCCC(=O)C1 ; O=C(O)c1cc(F)c(F)cc1I ; O=C1COCc2[nH]c(=O)c3cc(F)c(F)cc3c21]

Step i: 8,9-Difluoro-3,4-dihydro-2H-benzo[c]chromene-1,6-dione was synthesized in an analogous manner as described above from 4,5-difluoro-2-iodo-benzoic acid (IIc) and cyclohexane-1,3-dione (IIa). ¹H NMR (400 MHz, CDCl₃) δ 8.99 (dd, 1H), 8.04 (dd, 1H), 2.94 (t, 2H), 2.73-2.58 (m, 2H), 2.24-2.12 (m, 2H).

Step ii: 8,9-Difluoro-2,3,4,5-tetrahydrophenanthridine-1,6-dione was synthesized in an analogous manner as described above from 8,9-difluoro-3,4-dihydro-2H-benzo[c]chromene-1,6-dione and ammonium acetate. LCMS: m/z found 250.1 [M+H]⁺; RT=0.90 min (Method B); ¹H NMR (400 MHz, DMSO-d₆) δ 12.15 (s, 1H), 9.14 (dd, 1H), 8.06 (dd, 1H), 2.88 (t, 2H), 2.54 (dd, 2H), 2.02 (h, 2H).

### 8,9-Difluoro-1-(methylamino)-2,3,4,5-tetrahydro-1H-phenanthridin-6-one (Vj)

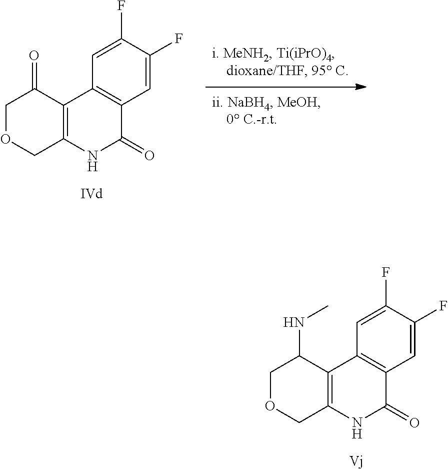 [SMILES: O=C1COCc2[nH]c(=O)c3cc(F)c(F)cc3c21 ; CNC1COCc2[nH]c(=O)c3cc(F)c(F)cc3c21]

Tetraisopropoxytitanium (0.48 mL, 1.58 mmol) was added to a mixture of 8,9-difluoro-2,3,4,5-tetrahydrophenanthridine-1,6-dione (0.1 g, 0.40 mmol) and a 2 M methylamine solution in THE (0.36 mL, 0.71 mmol) in 1,4-dioxane (5 mL). The mixture was stirred under nitrogen at room temperature for 2 h. An additional 0.1 mL of a 2 M solution of methylamine and 0.2 mL of tetraisopropoxytitanium were added to the reaction and stirring was continued at room temperature for 2 hours and then at 45° C. for a further 1 hours. The reaction mixture was diluted with 2 mL of anhydrous methanol and cooled in an ice bath. Sodium borohydride (30 mg, 0.79 mmol) was added in one portion, the reaction mixture was stirred for 5 minutes and the ice bath was removed. After an additional 15 min the reaction was quenched by the addition of brine (1.5 mL), diluted with 20 mL of ethyl acetate, and stirred for additional 15 min. The mixture was filtered through CELITE® and the filter cake was washed with 25 mL of ethyl acetate. The product was isolated by flash-chromatography (Silicagel, MeOH/DCM 0-10%) to provide 84 mg (80% yield) of 8,9-difluoro-1-(methylamino)-2,3,4,5-tetrahydro-1H-phenanthridin-6-one. LCMS: m/z found 265.2 [M+H]⁺, 234.1 [M−MeNH]⁺; RT=0.70 min, (Method B); ¹H NMR (400 MHz, CDCl₃) δ 8.02 (dd, 1H), 7.33 (dd, 1H), 3.65 (dd, 1H), 3.59-3.37 (bs, exchangeable protons), 2.59-2.45 (m, 5H), 2.21-2.11 (m, 1H), 1.96-1.79 (m, 1H), 1.78 (tt, 1H), 1.49 (tt, 1H).

### 3-(3-Chloro-4-fluorophenyl)-1-(8,9-difluoro-6-oxo-1,2,3,4,5,6-hexahydrophenanthridin-1-yl)-1-methylurea (Compound 15/Compound 38/Compound 39)

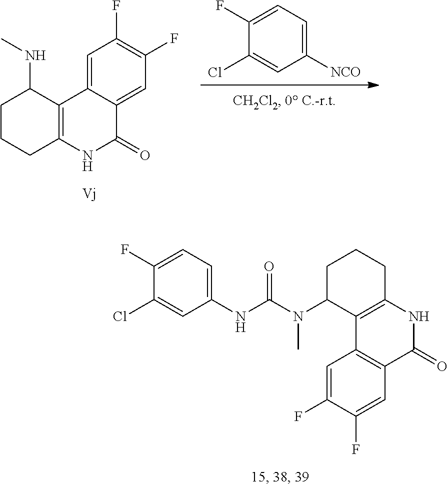

3-(3-Chloro-4-fluorophenyl)-1-(8,9-difluoro-6-oxo-1,2,3,4,5,6-hexahydrophenanthridin-1-yl)-1-methylurea (racemic) was synthesized in an analogous manner as described above from racemic 8,9-difluoro-1-(methylamino)-2,3,4,5-tetrahydro-1H-phenanthridin-6-one (Vj) and 2-chloro-1-fluoro-4-isocyanato-benzene. LCMS: m/z found 436.1/438.1 [M+H]⁺; RT=4.76 min, (Method A); ¹H NMR (400 MHz, DMSO-d₆) δ 11.54 (s, 1H), 8.50 (s, 1H), 8.07 (dd, 1H), 7.85 (dd, 1H), 7.52 (ddd, 1H), 7.38-7.26 (m, 2H), 5.56 (s, 1H), 2.71-2.63 (m, 2H), 2.63 (s, 3H), 2.56 (t, 1H), 2.01-1.89 (m, 1H), 1.84 (q, 1H), 1.74 (m, 1H). The enantiomers were subsequently separated by preparative SFC: Method isocratic, Mobile phase MeOH: CO₂ —40:60. Column: CHIRALPAK IC (30×150 mm) 5 m; 40% Methanol; Total flow: 90 g/min.

Enantiomer I (Compound 38). LCMS: m/z found 436.1/438.1 [M+H]⁺; RT=4.51 min, (Method A); Chiral analytical SFC: RT=1.40 min, Column: CHIRALPAK IC-3 (4.6×150 mm) 3 m; 40% Methanol; Total flow: 3 g/min.

Enantiomer II (Compound 39). LCMS: m/z found 436.2/438.1 [M+H]⁺; RT=4.51 min, (Method A); Chiral analytical SFC: RT=2.16 min, Column: CHIRALPAK IC-3 (4.6×150 mm) 3 m; 40% Methanol; Total flow: 3 g/min.

### 1-(8,9-Difluoro-6-oxo-1,2,3,4,5,6-hexahydrophenanthridin-1-yl)-3-(4-fluorophenyl)-1-methylurea (Compound 19)

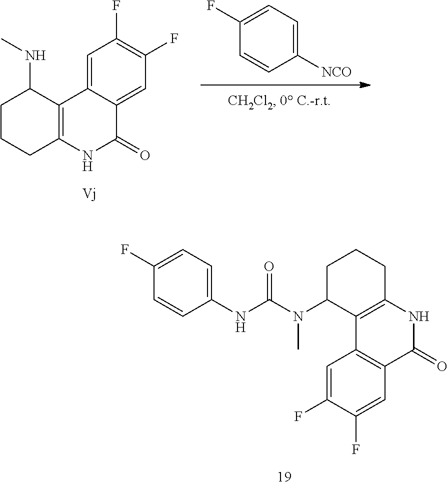

1-(8,9-Difluoro-6-oxo-1,2,3,4,5,6-hexahydrophenanthridin-1-yl)-3-(4-fluorophenyl)-1-methylurea was synthesized in an analogous manner as described above from racemic 8,9-difluoro-1-(methylamino)-2,3,4,5-tetrahydro-1H-phenanthridin-6-one (Vj) and 1-fluoro-4-isocyanato-benzene. LCMS: m/z found 402.2 [M+H]⁺; RT=4.21 min, (Method A); ¹H NMR (400 MHz, DMSO-d₆) δ 11.53 (s, 1H), 8.35 (s, 1H), 8.07 (dd, 1H), 7.60-7.49 (m, 2H), 7.35 (dd, 1H), 7.17-7.06 (m, 2H), 5.58 (d, 1H), 2.72-2.62 (m, 1H), 2.62 (s, 3H), 2.55 (m, 1H), 2.01-1.88 (m, 1H), 1.83 (m, 2H), 1.77-1.70 (m, 1H).

### 1-(8,9-Difluoro-6-oxo-1,2,3,4,5,6-hexahydrophenanthridin-1-yl)-1-methyl-3-(1-(trifluoromethyl)cyclopropyl)urea (Compound 144)

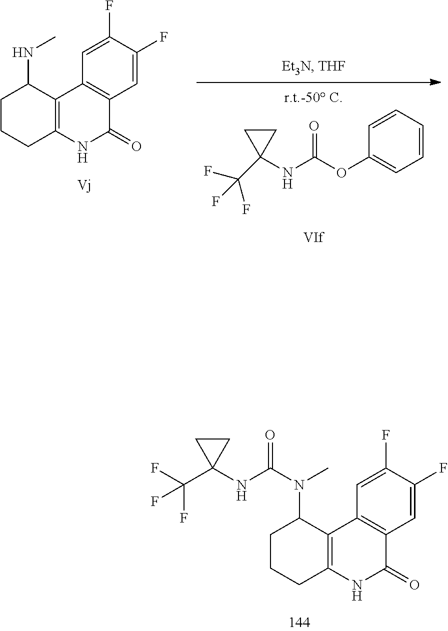 [SMILES: CNC1CCCc2[nH]c(=O)c3cc(F)c(F)cc3c21 ; O=C(NC1(C(F)(F)F)CC1)Oc1ccccc1 ; CN(C(=O)NC1(C(F)(F)F)CC1)C1CCCc2[nH]c(=O)c3cc(F)c(F)cc3c21]

Triethylamine (47 uL, 0.34 mmol) was added to a mixture of racemic 8,9-difluoro-1-(methylamino)-2,3,4,5-tetrahydro-1H-phenanthridin-6-one (Vj, 23.0 mg, 0.09 mmol) and phenyl N-[1-(trifluoromethyl)cyclopropyl]carbamate (VIf—prepared similarly to VIb—21.3 mg, 0.09 mmol) in 1 mL anhydrous THF, and the reaction mixture was stirred at room temperature for 5 min, then at 50° C. overnight. The reaction mixture was diluted with 30 mL EtOAc and washed with 0.2N HCl (10 mL), then with 5% aqueous NaHCO₃(15 mL), then with water, and brine, and dried over sodium sulfate. The organic solution was filtered, and the solvent was evaporated, and the residue was adsorbed onto Silicagel. The product was purified by flash-chromatography (Silicagel, EtOAc/hexanes 20-95%), and dried overnight under high vacuum, to provide 19.9 mg (55% yield) of 1-(8,9-difluoro-6-oxo-1,2,3,4,5,6-hexahydrophenanthridin-1-yl)-1-methyl-3-(1-(trifluoromethyl)cyclopropyl)urea. LCMS m/z found 416.2 [M+H]⁺; RT=3.30 min (Method A); ¹H NMR (400 MHz, DMSO-d₆) δ 11.48 (s, 1H), 8.05 (dd, 1H), 7.56-7.04 (m, 2H), 5.50 (s, 1H), 2.72-2.58 (m, 1H), 2.46 (d, 2H), 1.97-1.60 (m, 3H), 1.35-1.18 (m, 2H), 1.18-0.96 (m, 2H).

### 4,5-Dihydropyrano[3,4-c]isoquinoline-1,6-dione (IVe)

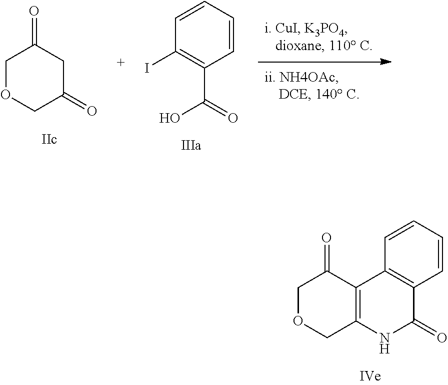 [SMILES: O=C1COCC(=O)C1 ; O=C(O)c1ccccc1I ; O=C1COCc2[nH]c(=O)c3ccccc3c21]

Step i: 2-Iodobenzoic acid (IIIa, 0.99 g, 4.0 mmol), tetrahydropyran-3,5-dione, (IIc, 0.55 g, 4.8 mmol), copper (I) iodide (76 mg, 0.4 mmol), and potassium phosphate (1.19 g, 5.6 mmol) were combined in a tube under a nitrogen atmosphere. Anhydrous 1,4-dioxane (10 mL) was added and the reaction tube was purged with nitrogen and stirred at room temperature for 30 min, and then at 110° C. for a further 3 hours. The reaction mixture was diluted with ethyl acetate (10 mL), filtered through CELITE® and the pad was washed with ethyl acetate (3×10 mL). The filtrate was evaporated under high vacuum and the residue was purified by flash chromatography (Silicagel, ethyl acetate/Hexanes 0-90%) to provide 0.41 g (47% yield) of 4H-pyrano[3,4-c]isochromene-1,6-dione. LCMS m/z found 217.1 [M+H]⁺; RT=0.94 min (Method B); ¹H NMR (400 MHz, CDCl₃) δ 8.92 (ddd, 1H), 8.30 (ddd, 1H), 7.84 (ddd, 1H), 7.59 (ddd, 1H), 4.74 (d, J=0.9 Hz, 2H), 4.36-4.30 (m, 2H).

Step ii: 4H-Pyrano[3,4-c]isochromene-1,6-dione (80 mg, 0.37 mmol) and ammonium acetate (0.17 g, 2.22 mmol) were stirred in 1,2-dichloroethane (4 mL) at 140° C. in a sealed tube for 7 hours. The reaction mixture was allowed to cool to room temperature, diluted with dichloromethane/methanol, and adsorbed onto Silicagel. The product was isolated by flash chromatography (Silicagel, dryloaded, MeOH/DCM 0-4%) to afford 60 mg (75% yield) of 4,5-dihydropyrano[3,4-c]isoquinoline-1,6-dione. LCMS: m/z found 216.1 [M+H]⁺; RT=0.87 min (Method B); ¹H NMR (400 MHz, DMSO-d₆) δ 12.12 (s, 1H), 9.02 (dq, 1H), 8.23 (ddd, 1H), 7.82 (ddd, 1H), 7.56 (ddd, 1H), 4.79 (d, 2H), 4.27 (d, 2H).

### 1-(Methylamino)-1,2,4,5-tetrahydropyrano[3,4-c]isoquinolin-6-one (Vk)

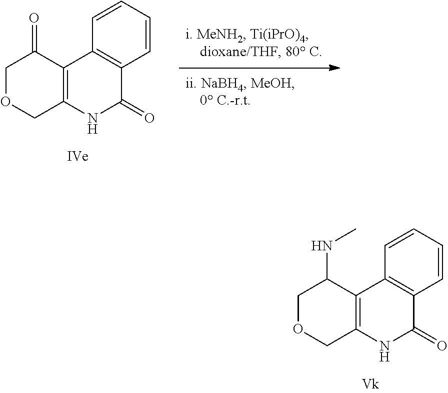 [SMILES: O=C1COCc2[nH]c(=O)c3ccccc3c21 ; CNC1COCc2[nH]c(=O)c3ccccc3c21]

Tetraisopropoxytitanium (0.56 mL, 1.86 mmol) was added to a mixture of 4,5-dihydropyrano[3,4-c]isoquinoline-1,6-dione (IVe, 0.10 g, 0.46 mmol) and a 2 M methylamine solution in THE (0.46 mL, 0.93 mmol), and 1,4-dioxane (5 mL). The mixture was stirred under nitrogen at 80° C. for 3 hours. The reaction mixture was diluted with 2 mL of anhydrous MeOH, cooled to 0° C., treated with sodium borohydride (35.2 mg, 0.93 mmol) and allowed to stir for 1 h. The reaction was quenched by addition of brine (1.5 mL), diluted with 20 mL of ethyl acetate, and stirred for additional 15 min. The mixture was filtered through CELITE®, and the filter cake was washed with an additional 25 mL ethyl acetate. The combined filtrate was dried over sodium sulfate, filtered, and the solvent was evaporated under reduced pressure. The product was isolated by flash-chromatography (Silicagel, MeOH/DCM 0-10%) to afford 86 mg (80% yield) of 1-(methylamino)-1,2,4,5-tetrahydropyrano[3,4-c]isoquinolin-6-one. LCMS: m/z found 200.1 [M−(MeNH)]⁺; RT=0.59 min, (Method B); ¹H NMR (400 MHz, CDCl₃) δ 11.61 (s, 1H), 8.46-8.39 (m, 1H), 7.79-7.68 (m, 2H), 7.49 (ddd, 1H), 4.72 (d, 1H), 4.60 (dd, 1H), 4.42 (d, 1H), 3.69-3.60 (m, 2H), 2.62 (s, 3H).

### 3-(3-Chloro-4-fluorophenyl)-1-methyl-1-(6-oxo-1,4,5,6-tetrahydro-2H-pyrano[3,4-c]isoquinolin-1-yl)urea (Compound 18/Compound 36/Compound 37)

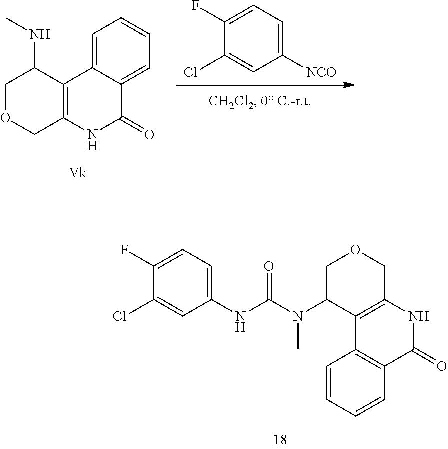

2-Chloro-1-fluoro-4-isocyanato-benzene (24.3 μL, 0.19 mmol) in 0.5 mL of dichloromethane was added slowly to a stirred mixture of 1-(methylamino)-1,2,4,5-tetrahydropyrano[3,4-c]isoquinolin-6-one (Vk, 61 mg, 0.26 mmol) in 2 mL dichloromethane at 0° C. The reaction was stirred for 1.5 hours allowing the cooling bath to warm to room temperature. MeOH (0.1 mL) was added and after 5 min the crude reaction mixture was directly adsorbed onto Silicagel. The product was isolated by flash chromatography (4 g Silicagel, 20%-80% ethyl acetate/hexane gradient), followed by trituration from acetate/hexane, and dried under high vacuum to provide racemic 3-(3-chloro-4-fluorophenyl)-1-methyl-1-(6-oxo-1,4,5,6-tetrahydro-2H-pyrano[3,4-c]isoquinolin-1-yl)urea (Compound 18, 49 mg, 46.0%). LCMS: m/z found 402.1/404.1 [M+H]⁺; RT=4.17 min, (Method A); ¹H NMR (400 MHz, Methanol-d₄) δ 8.34 (ddd, 1H), 7.80-7.66 (m, 2H), 7.62 (d, 1H), 7.53 (ddd, 1H), 7.39 (ddd, 1H), 7.18 (t, 1H), 5.55 (s, 1H), 4.67 (d, 1H), 4.54 (dd, 1H), 4.21 (dd, 1H), 4.01 (dd, 1H), 2.89 (s, 3H). The enantiomers were subsequently separated by preparative SFC: Method isocratic, Mobile phase MeOH: CO₂—40:60. Column: CHIRALPAK AD (30×150 mm) 5 m; Total flow: 90 g/min.

3-(3-Chloro-4-fluorophenyl)-1-methyl-1-(6-oxo-1,4,5,6-tetrahydro-2H-pyrano[3,4-c]isoquinolin-1-yl)urea: Enantiomer I (Compound 36). LCMS: m/z found 402.2/404.1 [M+H]⁺; RT=3.80 min, (Method A); Chiral analytical SFC: RT=1.77 min, Column: CHIRALPAK AD-3 (4.6×150 mm) 3 m; 40% Methanol; Total flow: 3 g/min.

3-(3-Chloro-4-fluorophenyl)-1-methyl-1-(6-oxo-1,4,5,6-tetrahydro-2H-pyrano[3,4-c]isoquinolin-1-yl)urea: Enantiomer II (Compound 37). LCMS: m/z found 402.2/404.1 [M+H]⁺; RT=3.79 min, (Method A); Chiral analytical SFC: RT=4.30 min, Column: CHIRALPAK AD-3 (4.6×150 mm) 3 m; 40% Methanol; Total flow: 3 g/min.

### 1-(Isobutylamino)-2,3,4,5-tetrahydro-1H-phenanthridin-6-one (Vel)

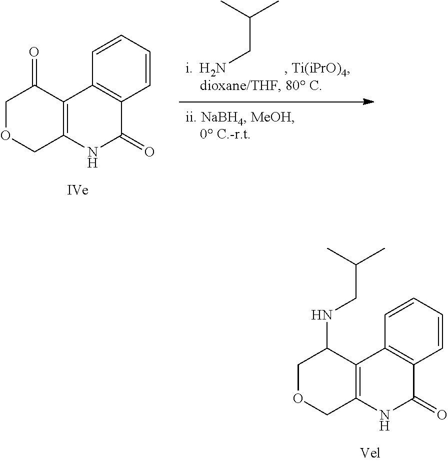

1-(Isobutylamino)-2,3,4,5-tetrahydro-1H-phenanthridin-6-one was synthesized in an analogous manner as described above from 4,5-dihydropyrano[3,4-c]isoquinoline-1,6-dione (IVe) and 2-methylpropan-1-amine. LCMS: m/z found 200.1 [M−(Me₂CHCH₂NH)]⁺; RT=0.77 min, (Method B); ¹H NMR (400 MHz, CDCl₃) δ 11.76 (s, 1H), 8.45-8.31 (m, 1H), 7.81-7.62 (m, 2H), 7.48 (ddd, 1H), 4.72 (d, 1H), 4.59 (dd, 1H), 4.37 (dd, 1H), 3.70-3.55 (m, 2H), 2.77 (dd, 1H), 2.50 (dd, 1H), 1.76 (dq, 1H), 0.95 (t, 6H).

### 3-(3-Chloro-4-fluorophenyl)-1-isobutyl-1-(6-oxo-1,4,5,6-tetrahydro-2H-pyrano[3,4-c]isoquinolin-1-yl)urea (Compound 30)

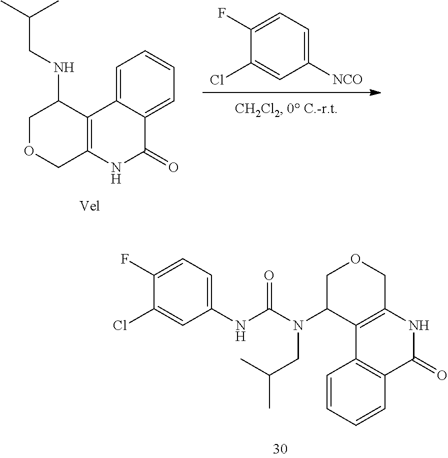

2-Chloro-1-fluoro-4-isocyanato-benzene (10 μL, 0.07 mmol) in 0.5 mL dichloromethane was added slowly to a stirred solution of 1-(isobutylamino)-1,2,4,5-tetrahydropyrano[3,4-c]isoquinolin-6-one (Vel, 28 mg, 0.10 mmol) in 1.5 mL dichloromethane at 0° C. The reaction was stirred for 1.5 hours allowing the cooling bath to warm to room temperature. MeOH (˜1.5 mL) was added and after 15 min the solvent was evaporated under vacuum to near dryness. The product was triturated from methanol and the product was collected by filtration, washed with methanol, then with ˜1:1 methanol/dichloromethane, then hexane, and high vacuum dried to provide 3-(3-chloro-4-fluorophenyl)-1-isobutyl-1-(6-oxo-1,4,5,6-tetrahydro-2H-pyrano[3,4-c]isoquinolin-1-yl)urea (33.3 mg, 73.0%). LCMS m/z found 444.2/446.2 [M+H]⁺; RT=4.67 min, (Method A); ¹H NMR (400 MHz, DMSO-d₆) δ 11.40 (s, 1H), 8.53 (s, 1H), 8.25-8.18 (m, 1H), 7.82 (dd, 1H), 7.75 (ddd, 1H), 7.57-7.44 (m, 3H), 7.33 (t, 1H), 5.42 (s, 1H), 4.57 (d, 1H), 4.44 (dd, 1H), 4.14-4.05 (m, 1H), 3.93 (dd, 1H), 3.33-3.20 (m, 1H), 2.96 (dd, 1H), 1.64 (p, 1H), 0.67 (d, 3H), 0.60 (d, 3H).

### 8,10-Difluoro-2,3,4,5-tetrahydrophenanthridine-1,6-dione (IVf)

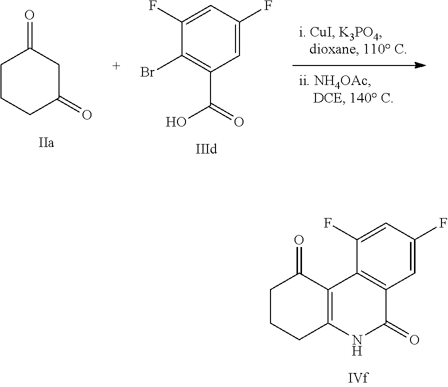 [SMILES: O=C1CCCC(=O)C1 ; O=C(O)c1cc(F)cc(F)c1Br ; O=C1CCCc2[nH]c(=O)c3cc(F)cc(F)c3c21]

8,10-Difluoro-2,3,4,5-tetrahydrophenanthridine-1,6-dione was synthesized in an analogous manner as described above, from cyclohexane-1,3-dione (IIa) and 2-bromo-3,5-difluoro-benzoic acid (IIId). LCMS: m/z found 250.1 [M+H]⁺; RT=0.85 min, (Method B); ¹H NMR (400 MHz, CDCl₃) δ 7.75 (dddd, 1H), 7.21-7.05 (m, 1H), 2.83-2.70 (m, 2H), 2.60 (td, 2H), 2.17-2.05 (m, 2H).

### 8,10-Difluoro-1-(methylamino)-2,3,4,5-tetrahydro-1H-phenanthridin-6-one (Vm)

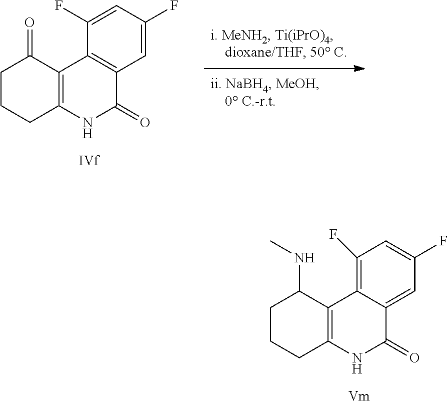 [SMILES: O=C1CCCc2[nH]c(=O)c3cc(F)cc(F)c3c21 ; CNC1CCCc2[nH]c(=O)c3cc(F)cc(F)c3c21]

8,10-Difluoro-1-(methylamino)-2,3,4,5-tetrahydro-1H-phenanthridin-6-one was synthesized in an analogous manner as described above from 8,10-difluoro-2,3,4,5-tetrahydrophenanthridine-1,6-dione (IVf) and methanamine. LCMS: m/z found 234.1 [M−MeNH]⁺; RT=0.71 min, (Method B); ¹H NMR (400 MHz, Methanol-d₄) δ 7.85 (dd, 1H), 7.36-7.25 (m, 1H), 4.29 (s, 1H), 2.70-2.60 (m, 2H), 2.54 (s, 3H), 2.28-2.17 (m, 1H), 2.02 (dddd, 1H), 1.83 (dt, 1H), 1.68 (tt, 1H).

### 3-(3-Chloro-4-fluorophenyl)-1-(8,10-difluoro-6-oxo-1,2,3,4,5,6-hexahydrophenanthridin-1-yl)-1-methylurea (Compound 20)

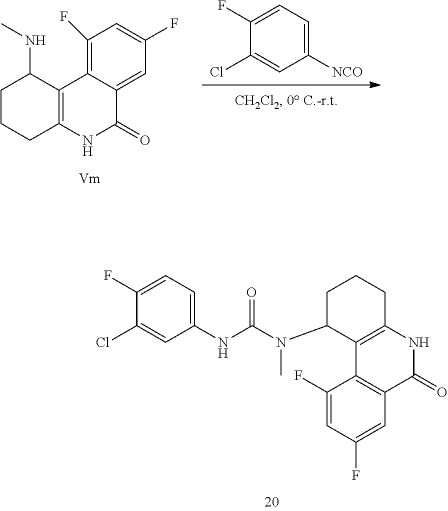

3-(3-Chloro-4-fluorophenyl)-1-(8,10-difluoro-6-oxo-1,2,3,4,5,6-hexahydrophenanthridin-1-yl)-1-methylurea was synthesized in an analogous manner as described above from racemic 8,10-difluoro-1-(methylamino)-2,3,4,5-tetrahydro-1H-phenanthridin-6-one (Vm) and 2-chloro-1-fluoro-4-isocyanato-benzene. LCMS: m/z found 438.1/440.1 [M+H]⁺; RT=4.21 min, (Method A); ¹H NMR (400 MHz, DMSO-d₆) δ 11.76 (s, 1H), 8.51 (s, 1H), 7.88-7.76 (m, 2H), 7.79-7.66 (m, 1H), 7.48 (ddd, 1H), 7.30 (t, 1H), 5.37 (s, 1H), 4.59 (d, 1H), 4.52-4.42 (m, 1H), 4.04 (dd, 1H), 3.85 (dd, 1H), 2.80 (s, 3H).

### 7,8,9,10-Tetrahydro-6H-cyclohepta[c]isoquinoline-5,11-dione (IVg)

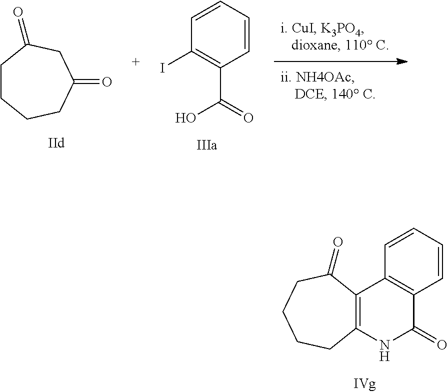 [SMILES: O=C1CCCCC(=O)C1 ; O=C(O)c1ccccc1I ; O=C1CCCCc2[nH]c(=O)c3ccccc3c21]

Step i: 7,8,9,10-Tetrahydrocyclohepta[c]isochromene-5,11-dione was synthesized in an analogous manner as described above from cycloheptane-1,3-dione (Id) and 2-iodobenzoic acid (IIIa). LCMS: m/z found 229.1 [M+H]⁺; RT=1.03 min, (Method B); ¹H NMR (400 MHz, CDCl₃) δ 8.29 (ddd, 1H), 8.11 (ddd, 1H), 7.79-7.68 (m, 1H), 7.51 (ddd, 1H), 3.00-2.88 (m, 2H), 2.87-2.75 (m, 2H), 2.04-1.90 (m, 4H).

Step ii: 7,8,9,10-Tetrahydro-6H-cyclohepta[c]isoquinoline-5,11-dione was synthesized in an analogous manner as described above from 7,8,9,10-tetrahydrocyclohepta[c]isochromene-5,11-dione and ammonium acetate. LCMS: m/z found 228.1 [M+H]⁺; RT=0.87 min, (Method B); ¹H NMR (400 MHz, CDCl₃) δ 11.81 (s, 1H), 8.43 (ddd, 1H), 8.30 (dt, 1H), 7.71 (ddd, 1H), 7.50 (ddd, 1H), 3.09-3.01 (m, 2H), 2.82-2.74 (m, 2H), 2.05-1.98 (m, 2H), 1.98-1.85 (m, 2H).

### 11-(Methylamino)-6,7,8,9,10,11-hexahydrocyclohepta[c]isoquinolin-5-one (Vn)

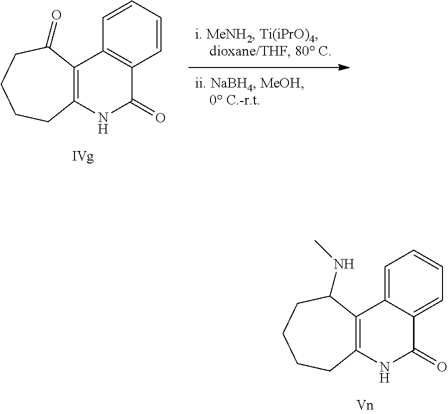 [SMILES: O=C1CCCCc2[nH]c(=O)c3ccccc3c21 ; CNC1CCCCc2[nH]c(=O)c3ccccc3c21]

11-(Methylamino)-6,7,8,9,10,11-hexahydrocyclohepta[c]isoquinolin-5-one was synthesized in an analogous manner as described above, from 7,8,9,10-tetrahydro-6H-cyclohepta[c]isoquinoline-5,11-dione (IVg) and methanamine. LCMS: m/z found 212.1 [M−MeNH]⁺; RT=0.71 min, (Method B); ¹H NMR (400 MHz, Methanol-d₄) δ 8.34 (ddd, 1H), 7.97 (dd, 1H), 7.77 (ddd, 1H), 7.48 (ddd, 1H), 4.60 (dd, 1H), 3.32-3.20 (m, 1H), 2.70-2.59 (m, 1H), 2.42 (s, 3H), 2.30-2.18 (m, 1H), 2.09-1.91 (m, 2H), 1.95-1.73 (m, 2H), 1.68-1.52 (m, 1H).

### 11-(3-Hydroxypropylamino)-6,7,8,9,10,11-hexahydrocyclohepta[c]isoquinolin-5-one (Vo)

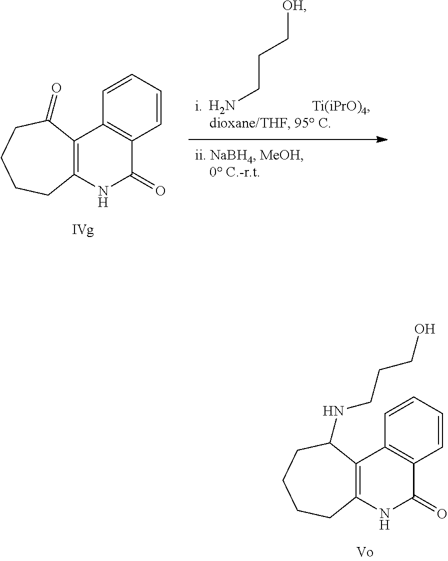 [SMILES: O=C1CCCCc2[nH]c(=O)c3ccccc3c21 ; NCCCO ; O=c1[nH]c2c(c3ccccc13)C(NCCCO)CCCC2]

11-(3-Hydroxypropylamino)-6,7,8,9,10,11-hexahydrocyclohepta[c]isoquinolin-5-one was synthesized in an analogous manner as described above, from 7,8,9,10-tetrahydro-6H-cyclohepta[c]isoquinoline-5,11-dione (IVg) and 3-aminopropan-1-ol. LCMS: m/z found 212.1 [M−(HO(CH₂)₃NH)]⁺; RT=0.71 min, (Method B); ¹H NMR (400 MHz, Methanol-d₄) δ 8.34 (dd, 1H), 7.90-7.83 (m, 1H), 7.71 (ddd, 1H), 7.44 (ddd, 1H), 4.52 (dd, 1H), 3.71-3.54 (m, 2H), 3.22 (ddd, 1H), 2.81 (dt, 1H), 2.70-2.51 (m, 2H), 2.25-2.13 (m, 1H), 2.07-1.89 (m, 2H), 1.82-1.64 (m, 4H), 1.68-1.51 (m, 1H).

### 3-(3-Chloro-4-fluorophenyl)-1-methyl-1-(5-oxo-6,7,8,9,10,11-hexahydro-5H-cyclohepta[c]isoquinolin-11-yl)urea (Compound 21)

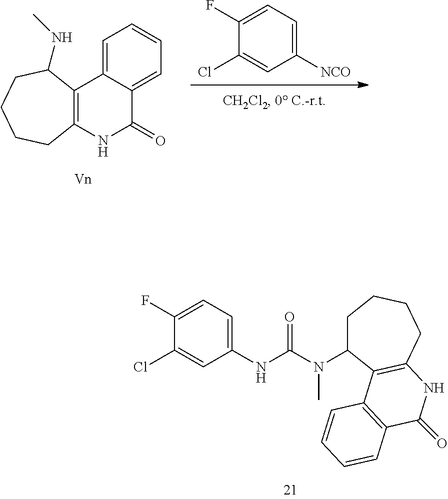

3-(3-Chloro-4-fluorophenyl)-1-methyl-1-(5-oxo-6,7,8,9,10,11-hexahydro-5H-cyclohepta[c]isoquinolin-11-yl)urea was synthesized in an analogous manner as described above from racemic 11-(methylamino)-6,7,8,9,10,11-hexahydrocyclohepta[c]isoquinolin-5-one (Vn) and 2-chloro-1-fluoro-4-isocyanato-benzene. LCMS: m/z found 414.2/416.2 [M+H]⁺; RT=4.61 min, (Method A); ¹H NMR (400 MHz, DMSO-d₆) δ 11.19 (s, 1H), 8.47 (s, 1H), 8.24-8.17 (m, 1H), 7.87 (ddd, 1H), 7.73-7.63 (m, 1H), 7.59 (d, 1H), 7.56-7.47 (m, 1H), 7.42 (ddd, 1H), 7.32 (td, 1H), 5.73 (t, 1H), 3.25-3.12 (m, 1H), 2.71 (s, 3H), 2.61 (d, 1H), 2.13-1.87 (m, 2H), 1.76 (d, 3H), 1.28 (dd, 1H).

### 3-(3-Chloro-4-fluorophenyl)-1-(3-hydroxypropyl)-1-(5-oxo-6,7,8,9,10,11-hexahydro-5H-cyclohepta[c]isoquinolin-11-yl)urea (Compound 22)

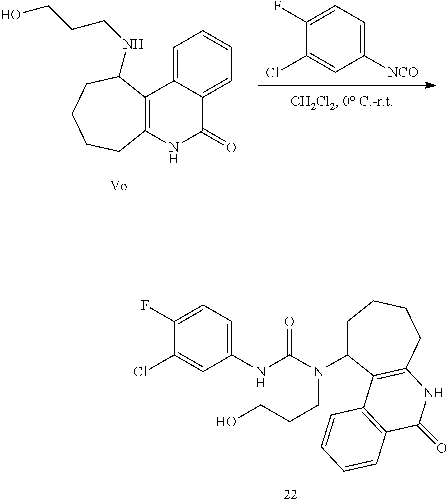

3-(3-Chloro-4-fluorophenyl)-1-(3-hydroxypropyl)-1-(5-oxo-6,7,8,9,10,11-hexahydro-5H-cyclohepta[c]isoquinolin-11-yl)urea was synthesized in an analogous manner as described above from racemic 11l-(3-hydroxypropylamino)-6,7,8,9,10,11-hexahydrocyclohepta[c]isoquinolin-5-one (Vo) and 2-chloro-1-fluoro-4-isocyanato-benzene. LCMS: m/z found 458.2/460.2 [M+H]⁺; RT=4.50 min, (Method A); ¹H NMR (400 MHz, DMSO-d₆) δ 11.17 (s, 1H), 8.73 (s, 1H), 8.19 (dd, 1H), 7.81 (dd, 1H), 7.73-7.64 (m, 1H), 7.57 (d, 1H), 7.48-7.37 (m, 2H), 7.33 (t, 1H), 5.73 (t, 1H), 5.06 (s, 1H), 3.33-3.15 (m, 2H), 3.15 (d, 2H), 2.59 (d, 1H), 2.15-2.06 (m, 1H), 1.92 (q, 1H), 1.77 (m, 3H), 1.21 (m, 4H).

### 8-Fluoro-4,5-dihydropyrano[3,4-c]isoquinoline-1,6-dione (IVh)

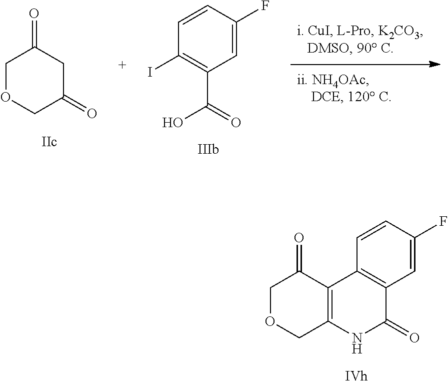 [SMILES: O=C1COCC(=O)C1 ; O=C(O)c1cc(F)ccc1I ; O=C1COCc2[nH]c(=O)c3cc(F)ccc3c21]

Step i: 5-Fluoro-2-iodo-benzoic acid (IIIb, 2.51 g, 9.44 mmol), tetrahydropyran-3,5-dione (IIc, 3.23 g, 28.31 mmol), copper (I) iodide (0.18 g, 0.94 mmol), L-proline (0.22 g, 1.89 mmol), and potassium dicarbonate (8.69 g, 37.74 mmol) were combined in a tube and evacuated and filled with nitrogen. Dry DMSO (30 mL) was added and the reaction mixture was purged with nitrogen, sealed, and stirred at room temperature for 10 min, and then at 90° C. for 2.5 hours. The reaction mixture was allowed to cool, diluted with 8 mL water, acidified with 2 M HCl to pH<2, and extracted with ethyl acetate (3×100 mL). The combined organic extracts were washed 3 times with water and once with brine, dried over sodium sulfate, and filtered. The solvent was evaporated under high vacuum to afford a crude product which was further dried under high vacuum overnight (when complete solidification occurred) and used in the next step without further purification.

Step ii: Crude 5-fluoro-2-(3-hydroxy-5-oxo-2H-pyran-4-yl)benzoic acid (2.38 g, 9.44 mmol) obtained in previous step and ammonium acetate (7.27 g, 94.37 mmol) were stirred in 1,2-dichloroethane (100 mL) at 120° C., in a sealed tube for 5 h. The reaction mixture was diluted with dichloromethane/methanol and adsorbed onto Silicagel, then submitted to flash chromatography (Silicagel, MeOH/DCM 0-10%). The desired product was further triturated with EtOAc/Hexanes to afford 8-fluoro-4,5-dihydropyrano[3,4-c]isoquinoline-1,6-dione (1.15 g, 52.3%). LCMS m/z found 234.1 [M+H]⁺; RT=0.77 min, (Method B); ¹H NMR (400 MHz, DMSO-d₆) δ 12.18 (s, 1H), 9.06 (dd, 1H), 7.86 (dd, 1H), 7.70 (ddd, 1H), 4.76 (s, 2H), 4.25 (s, 2H).

### 8-Fluoro-1-(methylamino)-1,2,4,5-tetrahydropyrano[3,4-c]isoquinolin-6-one (Vp)

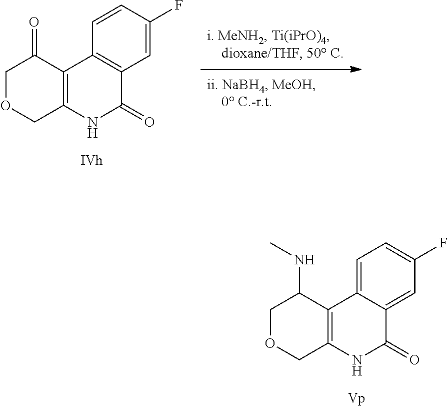 [SMILES: O=C1COCc2[nH]c(=O)c3cc(F)ccc3c21 ; CNC1COCc2[nH]c(=O)c3cc(F)ccc3c21]

8-Fluoro-1-(methylamino)-1,2,4,5-tetrahydropyrano[3,4-c]isoquinolin-6-one was synthesized in an analogous manner as described above, from 8-fluoro-4,5-dihydropyrano[3,4-c]isoquinoline-1,6-dione (IVh) and methanamine. LCMS: m/z found 249.2 [M+H]⁺; RT=0.49 min, (Method B); ¹H NMR (400 MHz, CDCl₃) δ 8.00-7.92 (m, 1H), 7.68 (dd, 1H), 7.42 (dddd, 1H), 4.60 (d, 1H), 4.53-4.44 (m, 1H), 4.36 (dd, 1H), 3.60 (dd, 1H), 3.55 (dt, 1H), 2.55 (d, 3H).

### 3-(3-Chloro-4-fluorophenyl)-1-(8-fluoro-6-oxo-1,4,5,6-tetrahydro-2H-pyrano[3,4-c]isoquinolin-1-yl)-1-methylurea (Compound 23/Compound 40/Compound 41)

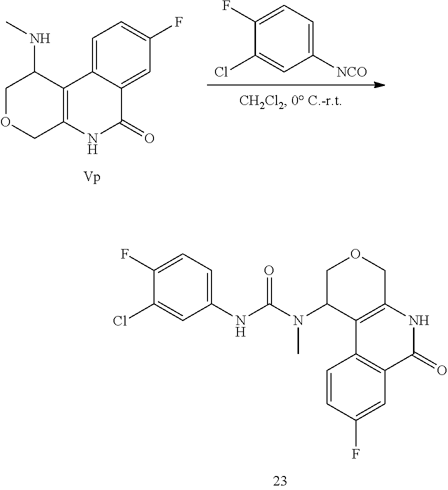

A solution of 2-chloro-1-fluoro-4-isocyanato-benzene (23 μL, 0.17 mmol) in 0.5 mL of dichloromethane was added dropwise to a stirred mixture of racemic 8-fluoro-1-(methylamino)-1,2,4,5-tetrahydropyrano[3,4-c]isoquinolin-6-one (Vp, 54 mg, 0.22 mmol) in 2 mL of dichloromethane at 0° C. The reaction was stirred for 1.5 hours while allowing it to warm to room temperature. Methanol (1.5 mL) was added and after 15 min the product was collected by filtration, washed with methanol, followed by 1:1 methanol/dichloromethane, and then with hexane, and dried under high vacuum at 50° C. to provide racemic 3-(3-chloro-4-fluorophenyl)-1-(8-fluoro-6-oxo-1,4,5,6-tetrahydro-2H-pyrano[3,4-c]isoquinolin-1-yl)-1-methylurea (Compound 23, 75.0 mg, 82%). LCMS: m/z found 420.2/422.1 [M+H]⁺; RT=4.28 min, (Method A); ¹H NMR (400 MHz, DMSO-d₆) δ 11.58 (s, 1H), 8.57 (s, 1H), 7.92-7.84 (m, 2H), 7.69 (td, 1H), 7.61-7.48 (m, 2H), 7.32 (t, 1H), 5.44 (s, 1H), 4.58 (d, 1H), 4.47-4.38 (m, 1H), 4.05 (d, 1H), 3.93 (dd, 1H), 2.80 (s, 3H).

The enantiomers were subsequently separated by preparative SFC: Method isocratic, Mobile phase MeOH: CO₂—40:60. Column: CHIRALPAK AD (30×150 mm) 5 m; Total flow: 90 g/min.

(R)-3-(3-Chloro-4-fluorophenyl)-1-(8-fluoro-6-oxo-1,4,5,6-tetrahydro-2H-pyrano[3,4-c]isoquinolin-1-yl)-1-methylurea: Enantiomer I (Compound 40). LCMS: m/z found 420.1/422.1 [M+H]⁺; RT=4.03 min, (Method A); Chiral analytical SFC: RT=1.37 min, Column: CHIRALPAK AD-3 (4.6×150 mm) 3 m; 40% Methanol; Total flow: 3 g/min.

(S)-3-(3-Chloro-4-fluorophenyl)-1-(8-fluoro-6-oxo-1,4,5,6-tetrahydro-2H-pyrano[3,4-c]isoquinolin-1-yl)-1-methylurea: Enantiomer II (Compound 41). LCMS: m/z found 420.1/422.1 [M+H]⁺; RT=4.02, (Method A); ¹H NMR (400 MHz, CDCl₃) δ 11.25 (s, 1H), 8.08 (dd, 1H), 7.69 (td, 2H), 7.46 (td, 1H), 7.24 (d, 1H), 7.10 (t, 1H), 6.42 (s, 1H), 5.71 (d, 1H), 4.78 (d, 1H), 4.62 (d, 1H), 4.30 (d, 1H), 3.99 (dd, 1H), 2.93 (s, 3H); Chiral analytical SFC: RT=7.15 min, Column: CHIRALPAK AD-3 (4.6×150 mm) 3 m; 40% Methanol; Total flow: 3 g/min.

Compound 41 was also prepared independently as described below and according to the general Scheme 3.

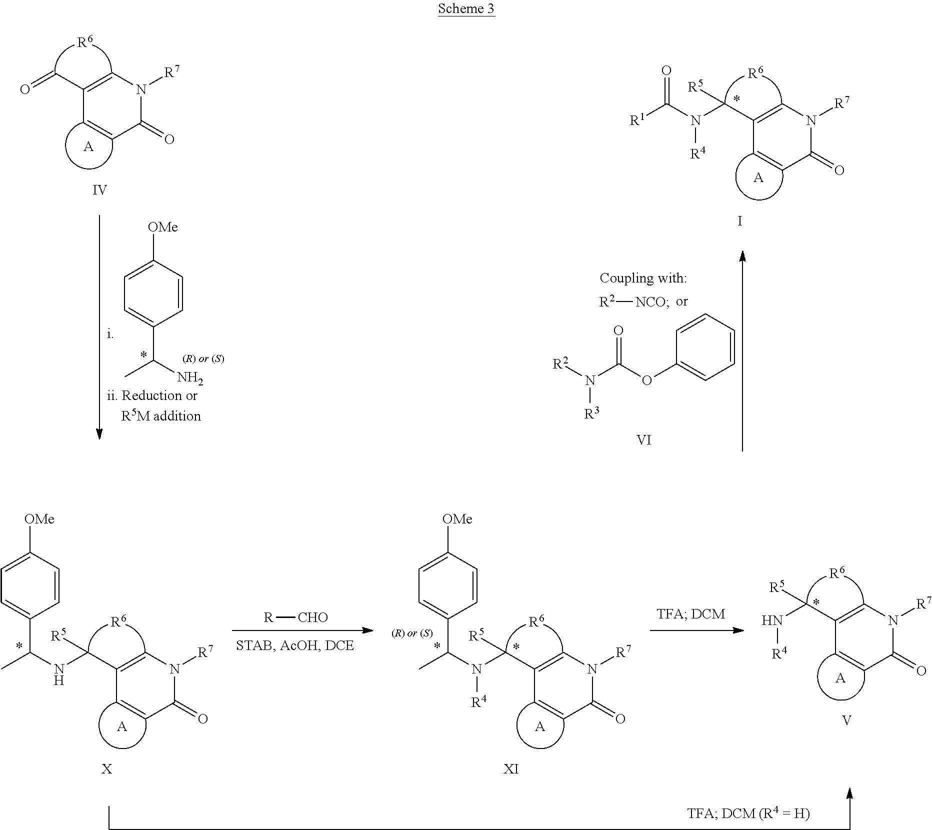

### (S)-8-Fluoro-1-(((R)-1-(4-methoxyphenyl)ethyl)amino)-1,5-dihydro-2H1-pyrano[3,4-c]isoquinolin-6(4H)-one (Xa)

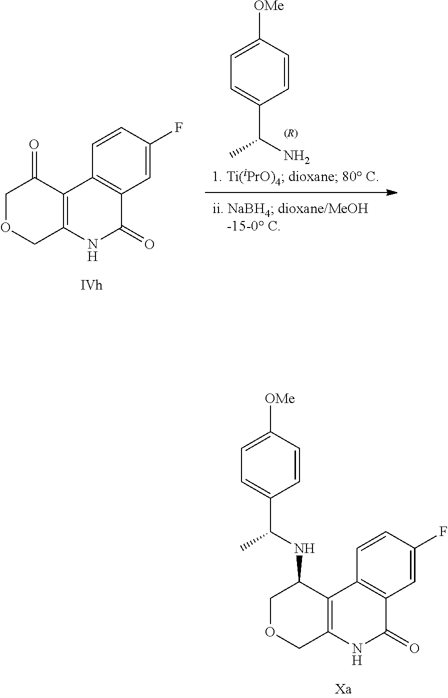

Tetraisopropoxytitanium (1.95 mL, 6.43 mmol) was added to a mixture of 8-fluoro-4,5-dihydropyrano[3,4-c]isoquinoline-1,6-dione (IVh, 500 mg, 2.14 mmol) and (1R)-1-(4-methoxyphenyl)ethanamine, (400 uL, 2.65 mmol), combined in 1,4-dioxane (5 mL). The mixture was stirred under nitrogen at 80° C. for 3 hours. The reaction mixture was diluted with 5 mL of dioxane, then cooled to −12° C. and treated with sodium borohydride (162 mg, 4.29 mmol) in 10 mL anhydrous MeOH. The reaction mixture was stirred for 1 hour, allowing the cooling bath to warm to 0° C. Stirring was continued for 30 min at 0° C., when LCMS indicated complete conversion of starting material. The reaction was quenched by the addition of 3 mL of brine and 15 mL of EtOAc at 0° C. The mixture was poured in a stirred mixture of 10 mL of brine and 40 mL of EtOAc and maintained at room temperature. After 15 min the mixture was filtered through CELITE®, and the filter cake was washed with an additional 40 mL of EtOAc. The combined filtrate was dried on sodium sulfate, filtered, and the solvent was evaporated under reduced pressure to provide a crude material as a mixture of diastereomers (d.r. ˜5:1, by LCMS DAD integration). The major diastereoisomer was isolated by flash chromatography (Silicagel, MeOH/DCM 0-2% 15 min gradient, then isocratic, then 3% to elute the minor isomer) to afford (S)-8-fluoro-1-(((R)-1-(4-methoxyphenyl)ethyl)amino)-1,5-dihydro-2H-pyrano[3,4-c]isoquinolin-6(4H)-one (Xa, 522.0 mg, 66% yield; d.r.=49:1 by LCMS DAD integration). LCMS: m/z found 369.3 [M+H]⁺; RT=0.59, (Method B); ¹H NMR (400 MHz, CDCl₃) δ 11.12 (s, 1H), 8.04 (dd, 1H), 7.87 (dd, 1H), 7.47 (dddd, 1H), 7.34-7.23 (m, 2H), 6.92-6.80 (m, 2H), 4.63 (d, 1H), 4.56-4.47 (m, 1H), 4.17-4.03 (m, 2H), 3.87 (t, 1H), 3.78 (d, 3H), 3.52 (dd, 1H), 1.45 (dd, 3H).

### (S)-8-Fluoro-1-(((R)-1-(4-methoxyphenyl)ethyl)(methyl)amino)-1,5-dihydro-2H-pyrano[3,4-c]isoquinolin-6(4H)-one (XIa)

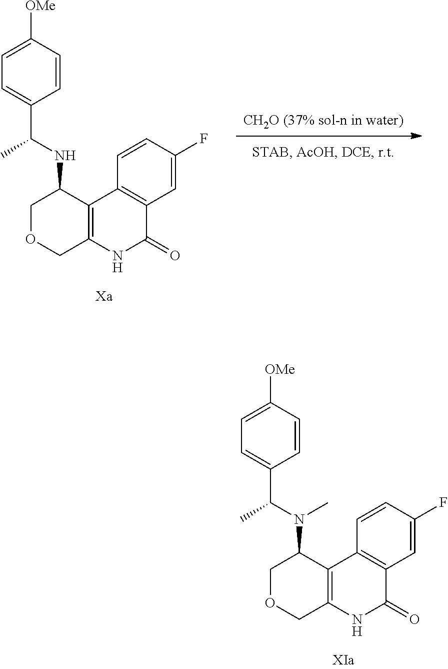 [SMILES: COc1ccc([C@@H](C)N[C@@H]2COCc3[nH]c(=O)c4cc(F)ccc4c32)cc1 ; COc1ccc([C@@H](C)N(C)[C@@H]2COCc3[nH]c(=O)c4cc(F)ccc4c32)cc1]

A mixture of diastereomerically pure (S)-8-fluoro-1-(((R)-1-(4-methoxyphenyl)ethyl)amino)-1,5-dihydro-2H-pyrano[3,4-c]isoquinolin-6(4H)-one (Xa, 120 mg, 0.33 mmol), an aqueous solution (37%) of formaldehyde (70 uL, 0.85 mmol), sodium triacetoxyborohydride (124 mg, 0.59 mmol), and acetic acid (34 uL, 0.59 mmol) were stirred in 1,2-dichloroethane (1.5 mL) overnight at room temperature. The reaction mixture was diluted with 5 mL of dichloromethane and neutralized with 1 M aqueous NaOH. The aqueous phase was extracted with dichloromethane twice more, and the combined organic extracts were washed with brine (1.5 mL), dried (sodium sulfate) and the solvent was evaporated under reduced pressure. The product was further purified by flash-chromatography (Silicagel, EtOAc/hexanes) to provide diastereomerically pure (S)-8-fluoro-1-(((R)-1-(4-methoxyphenyl)ethyl) (methyl)amino)-1,5-dihydro-2H-pyrano[3,4-c]isoquinolin-6(4H)-one (XIa, 80.6 mg, 65%). LCMS: m/z found 383.3 [M+H]⁺; RT=2.09, (Method A); ¹H NMR (400 MHz, CDCl₃) δ 11.05 (s, 1H), 8.23 (dd, 1H), 8.05 (dd, 1H), 7.48 (ddd, 1H), 7.19-7.11 (m, 2H), 6.83-6.74 (m, 2H), 4.67 (d, 1H), 4.57-4.48 (m, 1H), 4.46 (d, 1H), 4.20 (s, 1H), 3.92 (q, 1H), 3.77 (s, 3H), 3.63 (dd, 1H), 2.16 (s, 3H), 1.50 (d, 3H).

### (S)-3-(3-Chloro-4-fluorophenyl)-1-(8-fluoro-6-oxo-1,4,5,6-tetrahydro-2H-pyrano[3,4-c]isoquinolin-1-yl)-1-methylurea: Enantiomer II (Compound 41)

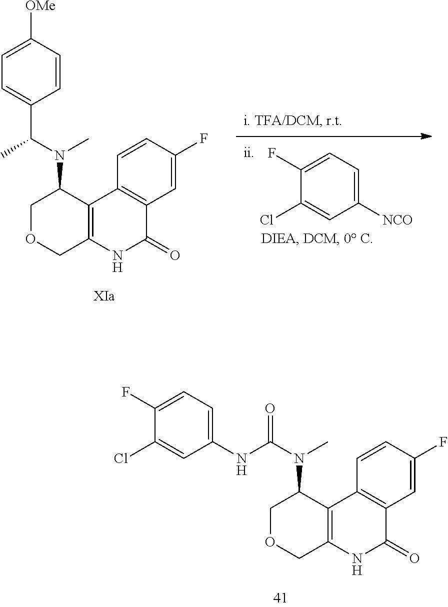

Step i: Diastereomerically pure (S)-8-fluoro-1-(((R)-1-(4-methoxyphenyl)ethyl)(methyl)amino)-1,5-dihydro-2H-pyrano[3,4-c]isoquinolin-6(4H)-one (XIa, 11 mg, 0.03 mmol) was stirred overnight with trifluoroacetic acid (0.12 mL, 1.05 mmol) in dichloromethane (0.12 mL), at room temperature. The reaction mixture was treated with 0.2 mL of MeOH, when the deep purple mixture transitioned almost instantaneously to a colorless, transparent solution. The volatiles were evaporated and the residue was azeotroped 2× with toluene and further dried on high vacuum to provide crude, enantiomerically pure (S)-8-fluoro-1-(methylamino)-1,2,4,5-tetrahydropyrano[3,4-c]isoquinolin-6-one (Vp), which was used in the next step without further purification. LCMS: m/z found 249.3 [M+H]⁺; RT=0.45, (Method B).

Step ii: Diisopropylethylamine (13 uL, 0.07 mmol) was added to the residue obtained in the step above, suspended in 0.5 mL of dichloromethane at 0° C. A solution of 2-chloro-1-fluoro-4-isocyanato-benzene (3 uL, 0.03 mmol) in 0.5 mL of dichloromethane was added slowly, and stirring was continued for 1 hour. The reaction was quenched with 0.5 mL of MeOH, and after 5 min the mixture was directly adsorbed onto Silicagel. The product was isolated by flash-chromatography (Silicagel, EtOAc/hexanes 10-95%), and dried under high vacuum to provide (S)-3-(3-chloro-4-fluorophenyl)-1-(8-fluoro-6-oxo-1,4,5,6-tetrahydro-2H-pyrano[3,4-c]isoquinolin-1-yl)-1-methylurea: Enantiomer II (Compound 41, 10 mg, 82.2%). LCMS m/z 420.2/422.2 [M+H]⁺; RT=4.02, (Method A); ¹H NMR (400 MHz, CDCl₃) δ 12.06 (s, 1H), 8.08 (ddd, 1H), 7.74-7.63 (m, 2H), 7.45 (tdd, 1H), 7.33-7.20 (m, 1H), 7.10 (td, 1H), 6.46 (s, 1H), 5.70 (d, 1H), 4.83 (d, 1H), 4.64 (dt, 1H), 4.34-4.26 (m, 1H), 3.99 (dd, 1H), 2.93 (d, 3H); Chiral analytical SFC: RT=4.83 min, Column: OD-10 analytical; 35% Methanol; Total flow: 3 g/min; ee=98%.

### (S)-3-(3-Cyano-4-fluorophenyl)-1-(8-fluoro-6-oxo-1,4,5,6-tetrahydro-2H-pyrano[3,4-c]isoquinolin-1-yl)-1-methylurea (Compound 64)

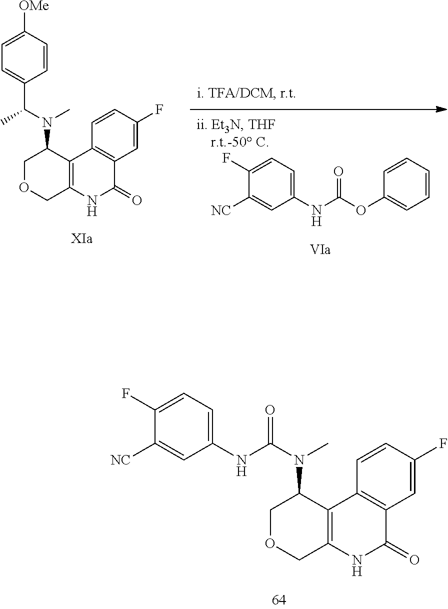 [SMILES: COc1ccc([C@@H](C)N(C)[C@@H]2COCc3[nH]c(=O)c4cc(F)ccc4c32)cc1 ; N#Cc1cc(NC(=O)Oc2ccccc2)ccc1F ; CN(C(=O)Nc1ccc(F)c(C#N)c1)[C@@H]1COCc2[nH]c(=O)c3cc(F)ccc3c21]

(S)-3-(3-Cyano-4-fluorophenyl)-1-(8-fluoro-6-oxo-1,4,5,6-tetrahydro-2H-pyrano[3,4-c]isoquinolin-1-yl)-1-methylurea was synthesized in an analogous manner as described for Compounds 41 and 70, from diastereomerically pure (S)-8-fluoro-1-(((R)-1-(4-methoxyphenyl)ethyl)(methyl)amino)-1,5-dihydro-2H-pyrano[3,4-c]isoquinolin-6(4H)-one (XIa) and phenyl N-(3-cyano-4-fluoro-phenyl)carbamate (VIa). LCMS m/z 411.3 [M+H]⁺; RT=0.80 min (Method B); ¹H NMR (400 MHz, DMSO-d₆) δ 11.59 (s, 1H), 8.75 (s, 1H), 8.09 (dd, 1H), 7.93-7.84 (m, 2H), 7.70 (td, 1H), 7.57 (dd, 1H), 7.46 (t, 1H), 5.45 (s, 1H), 4.58 (d, 1H), 4.48-4.38 (m, 1H), 4.06 (d, 1H), 3.94 (dd, 1H), 2.81 (s, 3H).

### (S)-1-(3-Chloro-4-fluorophenyl)-3-(8-fluoro-6-oxo-1,4,5,6-tetrahydro-2H-pyrano[3,4-c]isoquinolin-1-yl)urea (Compound 67)

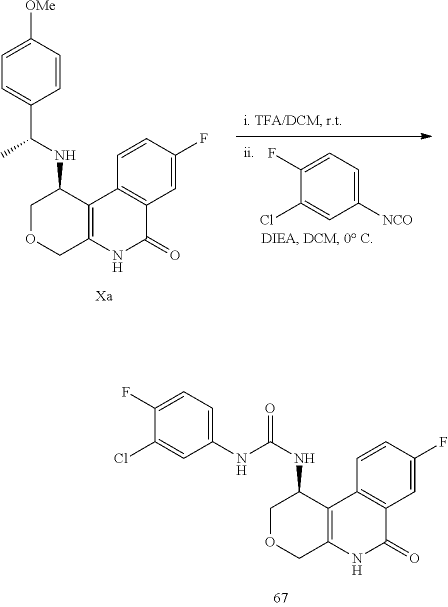

(S)-1-(3-Chloro-4-fluorophenyl)-3-(8-fluoro-6-oxo-1,4,5,6-tetrahydro-2H-pyrano[3,4-c]isoquinolin-1-yl)urea was synthesized in an analogous manner as described above for Compound 41, from diastereomerically pure (S)-8-fluoro-1-(((R)-1-(4-methoxyphenyl)ethyl)amino)-1,5-dihydro-2H-pyrano[3,4-c]isoquinolin-6(4H)-one (Xa). LCMS m/z 406.1/408.2 [M+H]⁺; RT=3.91 min (Method A); ¹H NMR (400 MHz, DMSO-d₆) δ 11.48 (s, 1H), 8.57 (s, 1H), 7.91-7.66 (m, 4H), 7.28 (t, 1H), 7.20 (ddd, 1H), 6.76 (d, 1H), 4.91 (d, 1H), 4.55-4.40 (m, 2H), 4.00 (dd, 1H), 3.83 (dd, 1H).

### 8-Fluoro-1-(isobutylamino)-1,2,4,5-tetrahydropyrano[3,4-c]isoquinolin-6-one (Vq)

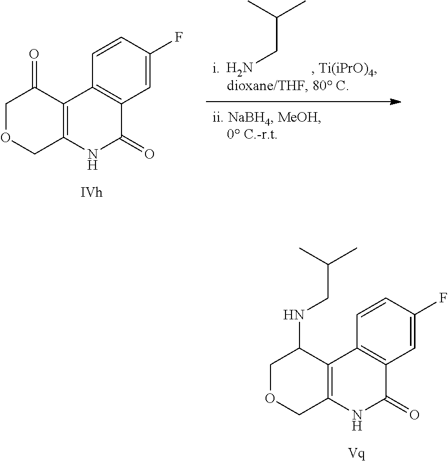

8-Fluoro-1-(isobutylamino)-1,2,4,5-tetrahydropyrano[3,4-c]isoquinolin-6-one was synthesized in an analogous manner as described above from 8-fluoro-4,5-dihydropyrano[3,4-c]isoquinoline-1,6-dione (IVh) and 2-methylpropan-1-amine. LCMS: m/z found 291.2.2 [M+H]⁺; RT=0.52 min, (Method B); ¹H NMR (400 MHz, CDCl₃) δ 11.52 (s, 1H), 8.06-7.98 (m, 1H), 7.82 (dd, 1H), 7.44 (dddd, 1H), 4.69 (d, 1H), 4.57 (d, 1H), 4.39 (d, 1H), 3.69-3.58 (m, 2H), 2.82-2.72 (m, 1H), 2.47 (dd, 1H), 1.74 (hept, 1H), 1.10-0.91 (m, 6H).

### 3-(3-Chloro-4-fluorophenyl)-1-(8-fluoro-6-oxo-1,4,5,6-tetrahydro-2H-pyrano[3,4-c]isoquinolin-1-yl)-1-isobutylurea (Compound 43)

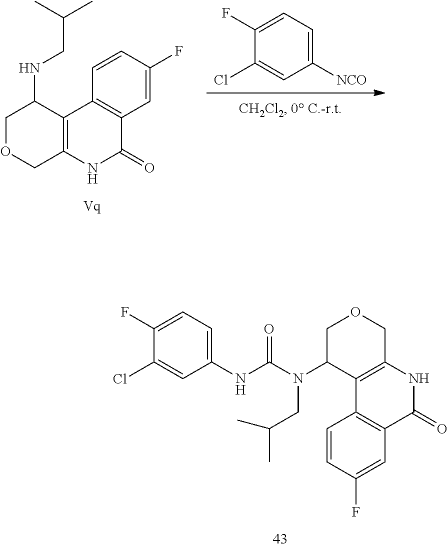

3-(3-Chloro-4-fluorophenyl)-1-(8-fluoro-6-oxo-1,4,5,6-tetrahydro-2H-pyrano[3,4-c]isoquinolin-1-yl)-1-isobutylurea was synthesized in an analogous manner as described above, from racemic 8-fluoro-1-(isobutylamino)-1,2,4,5-tetrahydropyrano[3,4-c]isoquinolin-6-one (Vq). LCMS: m/z found 462.3/464.3 [M+H]⁺; RT=4.83, (Method A); ¹H NMR (400 MHz, DMSO-d₆) δ 11.56 (s, 1H), 8.53 (s, 1H), 7.92-7.78 (m, 2H), 7.71 (td, 1H), 7.62 (dd, 1H), 7.49 (ddd, 1H), 7.33 (t, 1H), 5.44 (s, 1H), 4.57 (d, 1H), 4.43 (d, 1H), 4.09 (d, 1H), 3.93 (dd, 1H), 3.33-3.20 (m, 1H), 3.00 (dd, 1H), 1.61 (dt, 1H), 0.67 (d, 3H), 0.58 (d, 3H).

### 3-(3,4-Difluorophenyl)-1-(8-fluoro-6-oxo-2,4,5,6-tetrahydro-1H-pyrano[3,4-c]isoquinolin-1-yl)-1-isobutylurea (Compounds 99 and 100)

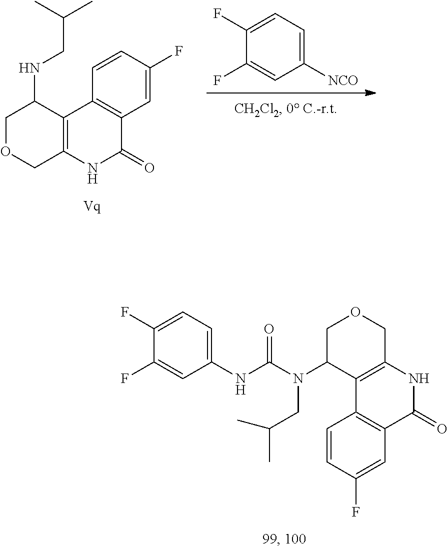

3-(3,4-Difluorophenyl)-1-(8-fluoro-6-oxo-2,4,5,6-tetrahydro-1H-pyrano[3,4-c]isoquinolin-1-yl)-1-isobutylurea was synthesized in an analogous manner as described above, from 8-fluoro-1-(isobutylamino)-1,2,4,5-tetrahydropyrano[3,4-c]isoquinolin-6-one (Vq) and 1,2-difluoro-4-isocyanatobenzene. The enantiomers were subsequently separated by preparative SFC: Method isocratic, Mobile phase MeOH: CO₂—20:80. Column: (R,R) Whelk-01 (30×250 mm), 5 μm, flow rate: 100 g/min.

Enantiomer I (Compound 99): LCMS: m/z found 446.3 [M+H]⁺, RT=4.48 min, (Method A); ¹H NMR (400 MHz, DMSO-d₆) δ 11.57 (br s, 1H), 8.53 (br s, 1H), 7.89-7.85 (m, 1H), 7.73-7.64 (m, 2H), 7.62-7.58 (m, 1H), 7.36-7.26 (m, 2H), 5.44-5.43 (m, 1H), 4.56 (d, 1H), 4.43 (d, 1H), 4.8 (d, 1H), 3.95-3.90 (m, 1H), 3.31-3.22 (m, 1H), 3.03-2.97 (m, 1H), 1.66-1.58 (m, 1H), 0.67 (d, 3H), 0.58 (d, 3H); Chiral analytical SFC: RT=6.35 min; Column ((R,R) Whelk-01 (4.6×250 mm) 3.5μ, 15% methanol, Flow rate: 3.0 g/min.

Enantiomer II (Compound 100): LCMS: m/z found 446.3 [M+H]⁺, RT=4.48, (Method A); ¹H NMR (400 MHz, DMSO-d₆): δ 11.57 (br s, 1H), 8.53 (br s, 1H), 7.89-7.85 (m, 1H), 7.73-7.64 (m, 2H), 7.62-7.58 (m, 1H), 7.36-7.26 (m, 2H), 5.44-5.43 (m, 1H), 4.56 (d, 1H), 4.43 (d, 1H), 4.8 (d, 1H), 3.95-3.90 (m, 1H), 3.31-3.22 (m, 1H), 3.03-2.97 (m, 1H), 1.66-1.58 (m, 1H), 0.67 (d, 3H), 0.58 (d, 3H); Chiral analytical SFC: RT=7.24 min; Column ((R,R) Whelk-01 (4.6×250 mm) 3.5μ, 15% methanol, Flow rate: 3.0 g/min.

### 1-(8-Fluoro-6-oxo-2,4,5,6-tetrahydro-1H-pyrano[3,4-c]isoquinolin-1-yl)-1-isobutyl-3-(3,4,5-trifluorophenyl)urea (Compounds 113 and 114)

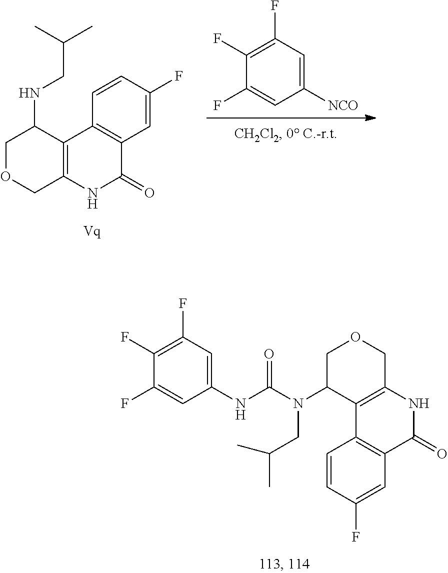

1-(8-Fluoro-6-oxo-2,4,5,6-tetrahydro-1H-pyrano[3,4-c]isoquinolin-1-yl)-1-isobutyl-3-(3,4,5-trifluorophenyl)urea was synthesized in an analogous manner as described above, from 8-fluoro-1-(isobutylamino)-1,2,4,5-tetrahydropyrano[3,4-c]isoquinolin-6-one (Vq) and 1,2,3-trifluoro-5-isocyanatobenzene. The enantiomers were subsequently separated by preparative SFC: Method isocratic, Mobile phase MeOH: CO₂—15:85. Column: Chiralpak IC (30×250 mm), 5 μm, flow rate: 90 g/min.

Enantiomer I (Compound 113): LCMS: m/z found 464.3 [M+H]⁺, RT=4.88 min, (Method A); ¹H NMR (400 MHz, DMSO-d₆) δ 11.57 (br s, 1H), 8.66 (br s, 1H), 7.88-7.85 (m, 1H), 7.71-7.66 (m, 1H), 7.59-7.48 (m, 3H), 5.42-5.41 (m, 1H), 4.56 (d, 1H), 4.43 (d, 1H), 4.09 (d, 1H), 3.94-3.90 (m, 1H), 3.31-3.22 (m, 1H), 3.02-2.96 (m, 1H), 1.65-1.58 (m, 1H), 0.66 (d, 3H), 0.58 (d, 3H); Chiral analytical SFC: RT=3.01 min, Column CHIRALPAK IC-3 (4.6×150 mm) 3 μm, 25% methanol, Flow rate: 3.0 g/min.

Enantiomer II (Compound 114): LCMS: m/z found 464.3 [M+H]⁺, RT=4.88 min, (Method A); ¹H NMR (400 MHz, DMSO-d₆) δ 11.57 (br s, 1H), 8.68 (br s, 1H), 7.88-7.85 (m, 1H), 7.71-7.66 (m, 1H), 7.59-7.48 (m, 3H), 5.42-5.41 (m, 1H), 4.56 (d, 1H), 4.43 (d, 1H), 4.09 (d, 1H), 3.94-3.90 (m, 1H), 3.31-3.22 (m, 1H), 3.02-2.96 (m, 1H), 1.65-1.58 (m, 1H), 0.66 (d, 3H), 0.58 (d, 3H); Chiral analytical SFC: RT=3.91 min, Column CHIRALPAK IC-3 (4.6×150 mm) 3 μm, 25% methanol, Flow rate: 3.0 g/min.

### 3-(3-Cyano-4-fluorophenyl)-1-(8-fluoro-6-oxo-1,4,5,6-tetrahydro-2H-pyrano[3,4-c]isoquinolin-1-yl)-1-isobutylurea (Compounds 115 and 116)

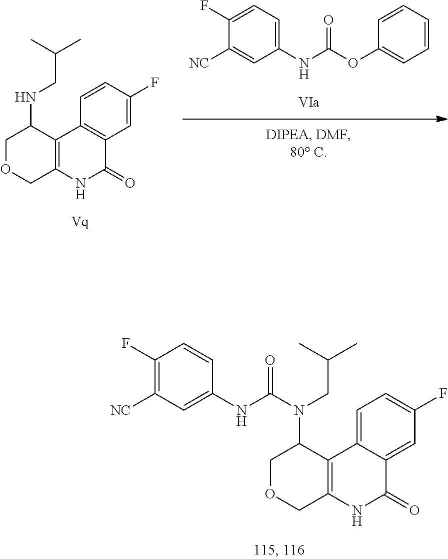 [SMILES: CC(C)CNC1COCc2[nH]c(=O)c3cc(F)ccc3c21 ; N#Cc1cc(NC(=O)Oc2ccccc2)ccc1F ; CC(C)CN(C(=O)Nc1ccc(F)c(C#N)c1)C1COCc2[nH]c(=O)c3cc(F)ccc3c21]

To a stirred solution of 100 mg (0.34 mmol) of 8-fluoro-1-(isobutylamino)-1,5-dihydro-2H-pyrano[3,4-c]isoquinolin-6(4H)-one (Vq) in 5 mL of DMF were added 0.13 mL (1.03 mmol) of DIPEA followed by 105.9 mg (0.43 mmol) of phenyl (3-cyano-4-fluorophenyl)carbamate (VIa) at room temperature. The reaction mixture was heated to 80° C. and stirred for 4 hours. The mixture was diluted with water (20 mL) and stirred at room temperature for a further 30 min. The solid formed from the reaction was collected by filtration and dried under vacuum. The obtained crude product was triturated with Et₂O (10 mL) and MTBE (10 mL) at room temperature, the solid filtered and dried under vacuum to afford 130 mg (0.28 mmol, 84%) of 3-(3-cyano-4-fluorophenyl)-1-(8-fluoro-6-oxo-1,4,5,6-tetrahydro-2H-pyrano[3,4-c]isoquinolin-1-yl)-1-isobutylurea. The enantiomers were subsequently separated by preparative SFC: Method isocratic, Mobile phase MeOH: CO₂ —45:55. Column: Chiralpak IG (30×250)mm, 5μ, flow rate: 100 g/min.

Enantiomer I (Compound 115): LCMS: m/z found 453.3 [M+H]⁺, RT=4.20 min, (Method A); ¹H NMR (400 MHz, DMSO-d₆) δ 11.57 (br s, 1H), 8.69 (br s, 1H), 8.05-8.02 (m, 1H), 7.89-7.83 (m, 2H), 7.72-7.66 (m, 1H), 7.62-7.58 (m, 1H), 7.46 (t, 1H), 5.44 (s, 1H), 4.57 (d, 1H), 4.43 (d, 1H), 4.10 (d, 1H), 3.95-3.91 (m, 1H), 3.30-3.23 (m, 1H), 3.03-2.97 (m, 1H), 1.64-1.58 (m, 1H), 0.67 (d, 3H), 0.58 (d, 3H); Chiral analytical SFC: RT=4.42 min; Column CHIRALPAK IC-3 (4.6×150 mm) 3 um, 20% methanol, Flow rate: 3.0 g/min.

Enantiomer II (Compound 116): LCMS: m/z found 453.3 [M+H]⁺, RT=4.20 min, (Method A); ¹H NMR (400 MHz, DMSO-d₆): δ 11.57 (br s, 1H), 8.69 (br s, 1H), 8.05-8.02 (m, 1H), 7.89-7.83 (m, 2H), 7.72-7.66 (m, 1H), 7.62-7.58 (m, 1H), 7.46 (t, 1H), 5.44 (s, 1H), 4.57 (d, 1H), 4.43 (d, 1H), 4.10 (d, 1H), 3.95-3.91 (m, 1H), 3.30-3.23 (m, 1H), 3.03-2.97 (m, 1H), 1.64-1.58 (m, 1H), 0.67 (d, 3H), 0.58 (d, 3H); Chiral analytical SFC: RT=5.85 min; Column CHIRALPAK IC-3 (4.6×150 mm) 3 um, 20% methanol, Flow rate: 3.0 g/min.

### 3-(4-Fluoro-3-methylphenyl)-1-(8-fluoro-6-oxo-1,4,5,6-tetrahydro-2H-pyrano[3,4-c]isoquinolin-1-yl)-1-isobutylurea (Compounds 131 and 132)

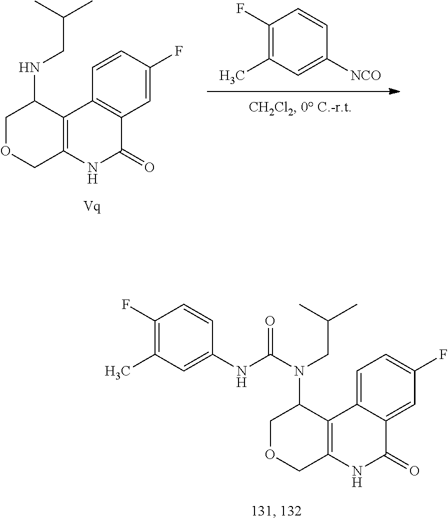

3-(4-Fluoro-3-methylphenyl)-1-(8-fluoro-6-oxo-1,4,5,6-tetrahydro-2H-pyrano[3,4-c]isoquinolin-1-yl)-1-isobutylurea was synthesized in an analogous manner as described above, from 8-fluoro-1-(isobutylamino)-1,2,4,5-tetrahydropyrano[3,4-c]isoquinolin-6-one (Vq) and 1-fluoro-4-isocyanato-2-methylbenzene. The enantiomers were subsequently separated by preparative SFC: Method isocratic, Mobile phase MeOH: CO₂—20:80. Column: Chiralpak IC (30×250 mm), 5μ, flow rate: 90 g/min.

Enantiomer I (Compound 131): LCMS: m/z found 442.3 [M+H]⁺, RT=4.50 min, (Method A); ¹H NMR (400 MHz, DMSO-d₆ δ 11.52 (br s, 1H), 8.28 (br s, 1H), 7.89-7.86 (m, 1H), 7.72-7.62 (m, 2H), 7.42-7.39 (m, 1H), 7.34-7.30 (m, 1H), 7.03 (t, 1H), 5.45-5.44 (m, 1H), 4.57 (d, 1H), 4.43 (d, 1H), 4.07 (d, 1H), 3.94-3.90 (m, 1H), 3.31-3.21 (m, 1H), 3.02-2.96 (m, 1H), 2.21 (s, 3H), 1.64-1.59 (m, 1H), 0.67 (d, 3H), 0.58 (d, 3H); Chiral analytical SFC: RT=3.49 min, Column: CHIRALPAK IC-3 (4.6×150 mm) 3 μm, 20% methanol, Flow rate: 3.0 g/min.

Enantiomer II (Compound 132): LCMS: m/z found 442.3 [M+H]⁺, RT=4.51 min, (Method A); ¹H NMR (400 MHz, DMSO-d₆ δ 11.52 (br s, 1H), 8.28 (br s, 1H), 7.89-7.86 (m, 1H), 7.72-7.62 (m, 2H), 7.42-7.39 (m, 1H), 7.34-7.30 (m, 1H), 7.03 (t, 1H), 5.45-5.44 (m, 1H), 4.57 (d, 1H), 4.43 (d, 1H), 4.07 (d, 1H), 3.94-3.90 (m, 1H), 3.31-3.21 (m, 1H), 3.02-2.96 (m, 1H), 2.21 (s, 3H), 1.64-1.59 (m, 1H), 0.67 (d, 3H), 0.58 (d, 3H); Chiral analytical SFC: RT=4.84 min, Column: CHIRALPAK IC-3 (4.6×150 mm) 3 μm, 20% methanol, Flow rate: 3.0 g/min.

### 1-(Ethylamino)-8-fluoro-1,2,4,5-tetrahydropyrano[3,4-c]isoquinolin-6-one (Vr)

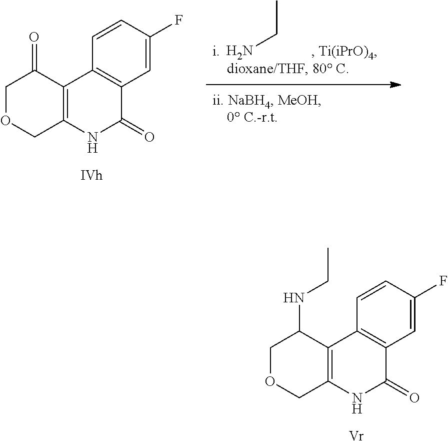

1-(Ethylamino)-8-fluoro-1,2,4,5-tetrahydropyrano[3,4-c]isoquinolin-6-one was synthesized in an analogous manner as described above from 8-fluoro-4,5-dihydropyrano[3,4-c]isoquinoline-1,6-dione (IVh) and ethanamine. LCMS: m/z found 263.2 [M+H]⁺; RT=0.44 min, (Method B); ¹H NMR (400 MHz, CDCl₃) δ 7.96-7.81 (m, 1H), 7.68 (dd, 1H), 7.41 (td, 1H), 4.60-4.32 (m, 2H), 4.30 (d, 1H), 3.65 (dd, 1H), 3.63-3.54 (m, 1H), 3.08 (br s, exch. protons), 2.89 (dq, 1H), 2.73 (dq, 1H), 1.14 (td, 3H).

### 3-(3-Chloro-4-fluorophenyl)-1-ethyl-1-(8-fluoro-6-oxo-1,4,5,6-tetrahydro-2H-pyrano[3,4-c]isoquinolin-1-yl)urea (Compound 44)

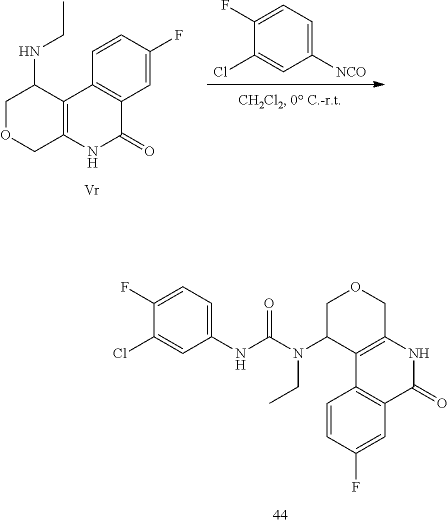

3-(3-Chloro-4-fluorophenyl)-1-ethyl-1-(8-fluoro-6-oxo-1,4,5,6-tetrahydro-2H-pyrano[3,4-c]isoquinolin-1-yl)urea was synthesized in an analogous manner as described above, from racemic 1-(ethylamino)-8-fluoro-1,2,4,5-tetrahydropyrano[3,4-c]isoquinolin-6-one (Vr). LCMS: m/z found 434.2/436.1 [M+H]⁺; RT=4.22 min (Method A); ¹H NMR (400 MHz, DMSO-d₆) δ 11.58 (s, 1H), 8.47 (s, 1H), 7.95-7.82 (m, 2H), 7.75-7.63 (m, 1H), 7.59-7.50 (m, 2H), 7.33 (td1H), 5.46 (s, 1H), 4.59 (d, 1H), 4.48-4.39 (m, 1H), 4.03 (d, 1H), 3.91 (dd, 1H), 3.48-3.34 (m, 1H), 3.23 (ddd, 1H), 0.84 (t, 3H).

### (S)-1-(Ethyl((R)-1-(4-methoxyphenyl)ethyl)amino)-8-fluoro-1,5-dihydro-2H-pyrano[3,4-c]isoquinolin-6(4H)-one (XIb)

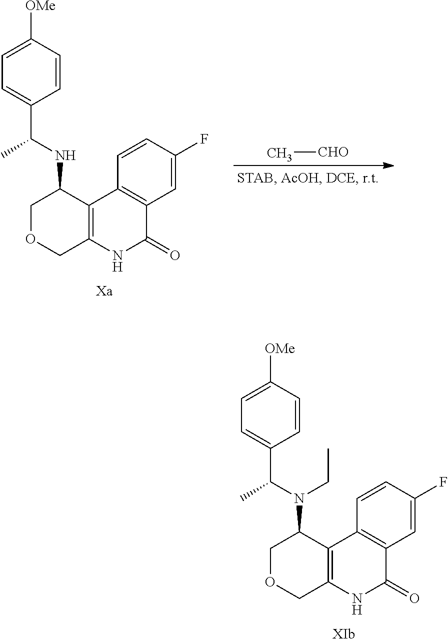

Diastereomerically pure (S)-1-(ethyl((R)-1-(4-methoxyphenyl)ethyl)amino)-8-fluoro-1,5-dihydro-2H-pyrano[3,4-c]isoquinolin-6(4H)-one was synthesized in an analogous manner as described above for XIa, in 86% yield, starting from diastereomerically pure (S)-8-fluoro-1-(((R)-1-(4-methoxyphenyl)ethyl)amino)-1,5-dihydro-2H-pyrano[3,4-c]isoquinolin-6(4H)-one (Xa) and acetaldehyde. LCMS m/z found 397.4 [M+H]⁺; RT=0.64 min (Method B); ¹H NMR (400 MHz, CDCl₃) δ 12.03 (s, 1H), 8.11 (dd, 1H), 8.02 (dd, 1H), 7.39 (ddd, 1H), 7.03 (d, 2H), 6.74-6.65 (m, 2H), 4.77 (d, 1H), 4.64-4.49 (m, 2H), 4.19-4.06 (m, 2H), 3.74 (s, 3H), 3.66 (dd, 1H), 2.83 (dq, 1H), 2.72 (dq, 1H), 1.48 (d, 3H), 0.90 (t, 3H).

### (S)-3-(3-Chloro-4-fluorophenyl)-1-ethyl-1-(8-fluoro-6-oxo-1,4,5,6-tetrahydro-2H-pyrano[3,4-c]isoquinolin-1-yl)urea (Compound 87)

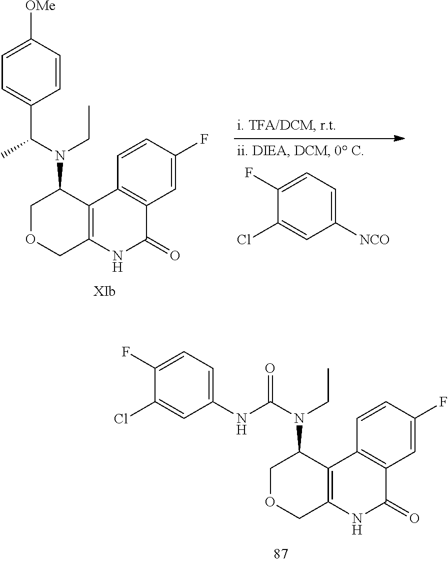

Optically pure (S)-3-(3-chloro-4-fluorophenyl)-1-ethyl-1-(8-fluoro-6-oxo-1,4,5,6-tetrahydro-2H-pyrano[3,4-c]isoquinolin-1-yl)urea was synthesized in an analogous manner as described above for Compound 41, from diastereomerically pure (S)-1-(ethyl((R)-1-(4-methoxyphenyl)ethyl)amino)-8-fluoro-1,5-dihydro-2H-pyrano[3,4-c]isoquinolin-6(4H)-one (XIb). LCMS m/z 434.3/436.3 [M+H]⁺; RT=6.73 min (Method A); ¹H NMR (400 MHz, DMSO-d₆) δ 11.58 (s, 1H), 8.47 (s, 1H), 7.93-7.84 (m, 2H), 7.70 (td, 1H), 7.55 (dddd, 2H), 7.33 (td, 1H), 5.46 (s, 1H), 4.59 (d, 1H), 4.44 (dd, 1H), 4.03 (d1H), 3.91 (dd, 1H), 3.41 (dd, 1H), 3.33-3.15 (m, 1H), 0.84 (t, 3H); Chiral analytical SFC: RT=7.74 min, Column: AD-analytical; 35% Methanol; Total flow: 3 g/min; ee=98.5%.

### 8-Fluoro-1-((3-Hydroxypropyl)amino)-1,5-dihydro-2H-pyrano[3,4-c]isoquinolin-6(4H)-one (Vpa)

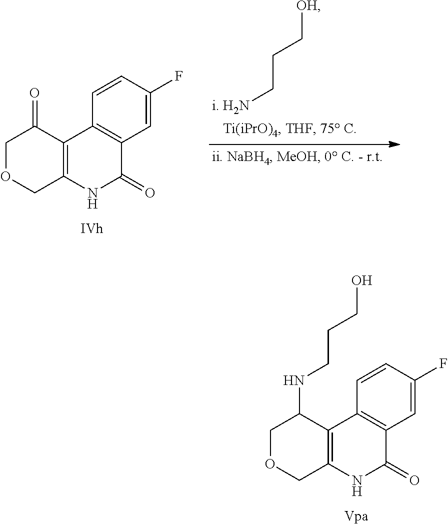

8-Fluoro-1-((3-hydroxypropyl)amino)-1,5-dihydro-2H-pyrano[3,4-c]isoquinolin-6(4H)-one was synthesized in an analogous manner as described above, from 8-fluoro-4,5-dihydropyrano[3,4-c]isoquinoline-1,6-dione (IVh) and 3-aminopropan-1-ol. LCMS: m/z found 293.1 [M+H]⁺; RT=1.70 min, (Method A); ¹H NMR (300 MHz, DMSO-d₆) δ 7.91-7.79 (m, 2H), 7.65-7.58 (m, 1H), 4.46-4.31 (m, 2H), 4.21 (d, 1H), 3.67 (s, 1H), 3.58-3.53 (m, 1H), 3.48-3.40 (m, 3H), 2.87-2.78 (m, 1H), 2.73-2.67 (m, 1H), 1.63-1.48 (m, 4H).

### 3-(3-Chloro-4-fluorophenyl)-1-(8-fluoro-6-oxo-1,4,5,6-tetrahydro-2H-pyrano[3,4-c]isoquinolin-1-yl)-1-(3-hydroxypropyl)urea (Compounds 109 and 110)

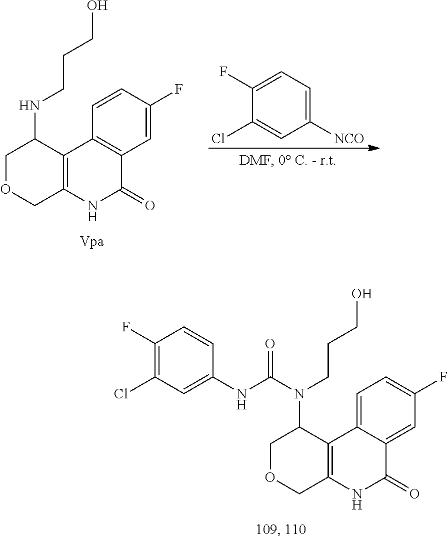

3-(3-Chloro-4-fluorophenyl)-1-(8-fluoro-6-oxo-1,4,5,6-tetrahydro-2H-pyrano[3,4-c]isoquinolin-1-yl)-1-(3-hydroxypropyl)urea was synthesized in an analogous manner as described above, except for using DMF as solvent, from 8-fluoro-1-((3-hydroxypropyl)amino)-1,5-dihydro-2H-pyrano[3,4-c]isoquinolin-6(4H)-one (Vpa) and 2-chloro-1-fluoro-4-isocyanatobenzene. The enantiomers were subsequently separated by preparative SFC: Method isocratic, Mobile phase Methanol: CO₂—25:75. Column: Chiralpak IG (30×250 mm), 5μ, flow rate: 90 g/min.

Enantiomer I (Compound 109): LCMS: m/z found 464.3/466.3 [M+H]⁺, RT=3.94 min, (Method A); ¹H NMR (400 MHz, DMSO-d₆): δ 11.56 (br s, 1H), 8.82 (br s, 1H), 7.89-7.86 (m, 1H), 7.84-7.81 (m, 1H), 7.72-7.66 (m, 1H), 7.56-7.53 (m, 1H), 7.46-7.42 (m, 1H), 7.33 (t, 1H), 5.47-5.46 (m, 1H), 5.02 (br s, 1H), 4.58 (d, 1H), 4.44 (d, 1H), 4.04 (d, 1H), 3.93-3.89 (m, 1H), 3.50-3.42 (m, 1H), 3.25-3.17 (m, 3H), 1.41-1.31 (m, 2H); Chiral analytical SFC: RT=2.04 min, Column CHIRALPAK IG-3 (4.6×150 mm) 3 μm, 25% methanol, Flow rate: 3.0 g/min.

Enantiomer II (Compound 110): LCMS: m/z found 464.2/466.2 [M+H]⁺, RT=3.93 min, (Method A); ¹H NMR (400 MHz, DMSO-d₆): δ 11.56 (br s, 1H), 8.82 (br s, 1H), 7.89-7.86 (m, 1H), 7.84-7.81 (m, 1H), 7.72-7.66 (m, 1H), 7.56-7.53 (m, 1H), 7.46-7.42 (m, 1H), 7.33 (t, 1H), 5.47-5.46 (m, 1H), 5.02 (br s, 1H), 4.58 (d, 1H), 4.44 (d, 1H), 4.04 (d, 1H), 3.93-3.89 (m, 1H), 3.50-3.42 (m, 1H), 3.25-3.17 (m, 3H), 1.41-1.31 (m, 2H); Chiral analytical SFC: RT=3.0 min, Column CHIRALPAK IG-3 (4.6×150 mm) 3 μm, 25% methanol, Flow rate: 3.0 g/min.

### 8-Fluoro-1-((2-hydroxy-2-methylpropyl)amino)-1,5-dihydro-2H-pyrano[3,4-c]isoquinolin-6(4H)-one (Vpb)

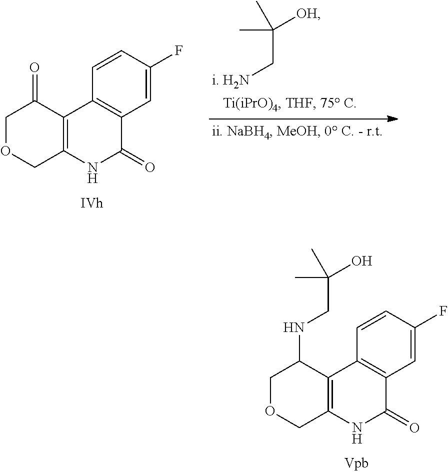

8-Fluoro-1-((2-hydroxy-2-methylpropyl)amino)-1,5-dihydro-2H-pyrano[3,4-c]isoquinolin-6(4H)-one was synthesized in an analogous manner as described above, from 8-fluoro-4,5-dihydropyrano[3,4-c]isoquinoline-1,6-dione (IVh) and 1-amino-2-methylpropan-2-ol. LCMS: m/z found 307.22 [M+H]⁺; RT=1.42 min, (Method A); ¹H NMR (300 MHz, DMSO-d₆) δ 7.91-7.79 (m, 2H), 7.65-7.58 (m, 1H), 4.46-4.31 (m, 2H), 4.21 (d, 1H), 3.67 (s, 1H), 3.58-3.53 (m, 1H), 3.48-3.40 (m, 3H), 2.87-2.78 (m, 1H), 2.73-2.67 (m, 1H), 1.63-1.48 (m, 4H).

### 3-(3-Chloro-4-fluorophenyl)-1-(8-fluoro-6-oxo-1,4,5,6-tetrahydro-2H-pyrano[3,4-c]isoquinolin-1-yl)-1-(2-hydroxy-2-methylpropyl)urea (Compounds 111 and 112)

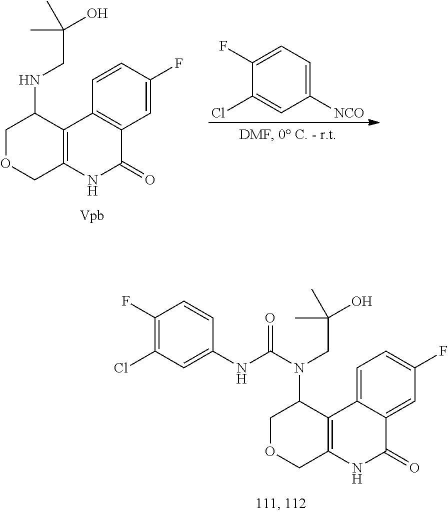

3-(3-Chloro-4-fluorophenyl)-1-(8-fluoro-6-oxo-1,4,5,6-tetrahydro-2H-pyrano[3,4-c]isoquinolin-1-yl)-1-(2-hydroxy-2-methylpropyl)urea was synthesized in an analogous manner as described above, from 8-fluoro-1-((2-hydroxy-2-methylpropyl)amino)-1,5-dihydro-2H-pyrano[3,4-c]isoquinolin-6(4H)-one (Vpb) and 2-chloro-1-fluoro-4-isocyanatobenzene. The product was purified by preparative HPLC (Column: SYMMETRY C18 (300×19) mm 7 u; Mobile phase A: 10 mM Ammonium Bicarbonate (Aq); Mobile phase B: Acetonitrile; Method T/% B=0.1/40, 11/70, 11.1/100, 13/100, 13.1/40, 15/40 Flow rate: 19 mL/min. The enantiomers were subsequently separated by preparative SFC: Method isocratic, Mobile phase Methanol: CO₂—15:85. Column: CHIRALPAK-IC (30×250 mm), 5μ, flow rate: 100 g/min.

Enantiomer I (Compound 111): LCMS: m/z found 478.3/480.2 [M+H]⁺, RT=4.36 min, (Method A); ¹H NMR (400 MHz, DMSO-d₆): δ 11.56 (br s, 1H), 10.70 (br s, 1H), 7.88-7.84 (m, 1H), 7.80-7.77 (m, 1H), 7.71-7.61 (m, 2H), 7.34 (t, 1H), 7.28-7.23 (m, 1H), 6.16 (br s, 1H), 5.65-5.64 (m, 1H), 4.58 (d, 1H), 4.42 (d, 1H), 4.10 (d, 1H), 3.95-3.91 (m, 1H), 3.44 (d, 1H), 3.34-3.31 (m, 1H), 1.12 (s, 3H), 0.57 (s, 3H); Chiral analytical SFC: RT=2.66 min, Column CHIRALPAK IC-3 (4.6×150 mm) 3 μm, 20% methanol, Flow rate: 3.0 g/min.

Enantiomer II (Compound 112): LCMS: m/z found 478.3/480.2 [M+H]⁺, RT=4.36 min, (Method A); ¹H NMR (400 MHz, DMSO-d₆): δ 11.56 (br s, 1H), 10.70 (br s, 1H), 7.88-7.84 (m, 1H), 7.80-7.77 (m, 1H), 7.71-7.61 (m, 2H), 7.34 (t, 1H), 7.28-7.23 (m, 1H), 6.16 (br s, 1H), 5.65-5.64 (m, 1H), 4.58 (d, 1H), 4.42 (d, 1H), 4.10 (d, 1H), 3.95-3.91 (m, 1H), 3.44 (d, 1H), 3.34-3.31 (m, 1H), 1.12 (s, 3H), 0.57 (s, 3H); Chiral analytical SFC: RT=3.94 min, Column CHIRALPAK IC-3 (4.6×150 mm) 3 μm, 20% methanol, Flow rate: 3.0 g/min.

### 3-(4-Fluoro-3-methylphenyl)-1-(8-fluoro-6-oxo-1,4,5,6-tetrahydro-2H-pyrano[3,4-c]isoquinolin-1-yl)-1-methylurea (Compound 57)

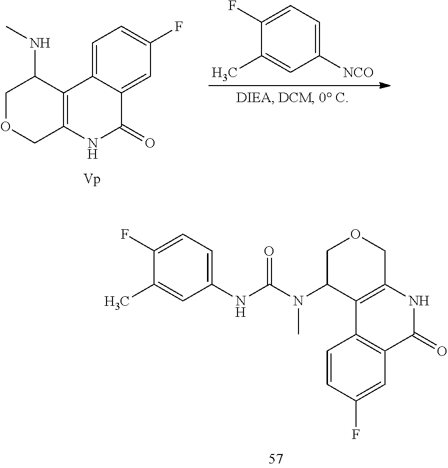

3-(4-Fluoro-3-methylphenyl)-1-(8-fluoro-6-oxo-1,4,5,6-tetrahydro-2H-pyrano[3,4-c]isoquinolin-1-yl)-1-methylurea was synthesized in an analogous manner as described above for Compound 41, from 8-fluoro-1-(methylamino)-1,2,4,5-tetrahydropyrano[3,4-c]isoquinolin-6-one trifluoroacetate salt (Vp) and 1-fluoro-4-isocyanato-2-methyl-benzene. LCMS m/z found 400.3 [M+H]⁺; RT=3.69 min (Method A); ¹H NMR (400 MHz, DMSO-d6) δ 11.57 (s, 1H), 8.34 (s, 1H), 7.88 (dd, 1H), 7.74-7.64 (m, 1H), 7.59 (dd, 1H), 7.51-7.43 (m, 1H), 7.36 (ddd, 1H), 7.03 (t, 1H), 6.77 (s, 3H), 5.45 (s, 1H), 4.58 (d, 1H), 4.47-4.38 (m, 1H), 4.03 (d, 1H), 3.92 (dd, 1H), 2.81-2.76 (m, 3H), 2.21 (d, 3H).

### 3-(3,5-Dichloro-4-fluorophenyl)-1-(8-fluoro-6-oxo-1,4,5,6-tetrahydro-2H-pyrano[3,4-c]isoquinolin-1-yl)-1-methylurea (Compounds 97 and 98)

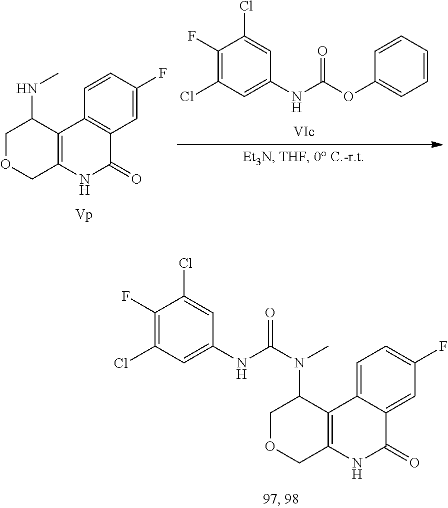

To a stirred solution of 8-fluoro-1-(methylamino)-1,5-dihydro-2H-pyrano[3,4-c]isoquinolin-6(4H)-one (Vp, 0.18 g, 0.72 mmol) in 5 mL of THE at 0° C. were added 0.22 g (2.17 mmol) of triethylamine followed by 0.22 g (0.72 mmol) of phenyl (3,5-dichloro-4-fluorophenyl)carbamate (VIc, prepared similarly to VIb) and stirring was continued at room temperature for 16 hours. The mixture was poured in to ice cold water (50 mL), and the precipitated solid was collected by filtration, washed with water (10 mL) and n-pentane (10 mL) and dried under vacuum to afford 105 mg (0.23 mmol, 32%) of 3-(3,5-dichloro-4-fluorophenyl)-1-(8-fluoro-6-oxo-1,4,5,6-tetrahydro-2H-pyrano[3,4-c]isoquinolin-1-yl)-1-methylurea. The enantiomers were subsequently separated by preparative SFC: Method isocratic, Mobile phase MeOH: CO₂—50:50. Column: Chiralcel OD-H (30×250 mm), 5μ, flow rate: 70 g/min.

Enantiomer I (Compound 97): LCMS: m/z found 454.2/456.2 [M+H]⁺, RT=4.70 min, (Method A); ¹H NMR (400 MHz, DMSO-d₆): δ 11.57 (br s, 1H), 8.69 (br s, 1H) 7.89-7.83 (m, 3H), 7.72-7.65 (m, 1H), 7.55-7.51 (m, 1H), 5.43-5.42 (m, 1H), 4.57 (d, 1H), 4.42 (d, 1H), 4.05 (d, 1H), 3.95-3.91 (m, 1H), 2.79 (s, 3H); Chiral analytical SFC: RT=1.31 min, Column CHIRALCEL OD-3 (4.6×150 mm) 3 m; 40% Methanol, Flow rate: 3.0 g/min.

Enantiomer II (Compound 98): LCMS: m/z found 454.3/456.2 [M+H]⁺, RT=4.70 min, (Method A); ¹H NMR (400 MHz, DMSO-d₆): δ 11.57 (br s, 1H), 8.69 (br s, 1H) 7.89-7.83 (m, 3H), 7.72-7.65 (m, 1H), 7.55-7.51 (m, 1H), 5.43-5.42 (m, 1H), 4.57 (d, 1H), 4.42 (d, 1H), 4.05 (d, 1H), 3.95-3.91 (m, 1H), 2.79 (s, 3H); UPLC: 99.66%, RT=3.43 min; Chiral analytical SFC: RT=2.03 min Column: Chiralcel OD-H (30×250 mm), 5μ, flow rate: 70 g/min.

### 3-(3-Chloro-4,5-difluorophenyl)-1-(8-fluoro-6-oxo-1,4,5,6-tetrahydro-2H-pyrano[3,4-c]isoquinolin-1-yl)-1-methylurea (Compounds 107 and 108)

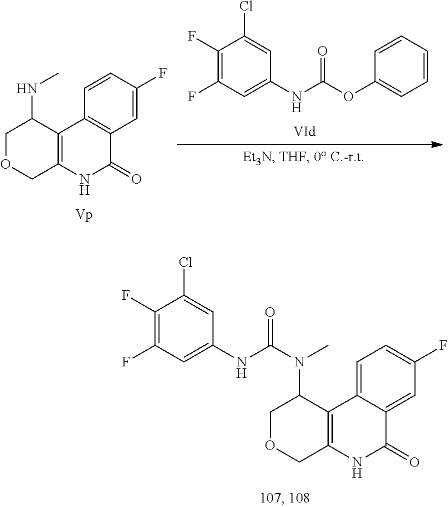

3-(3-Chloro-4,5-difluorophenyl)-1-(8-fluoro-6-oxo-1,4,5,6-tetrahydro-2H-pyrano[3,4-c]isoquinolin-1-yl)-1-methylurea was synthesized in an analogous manner as described above, from 8-fluoro-1-(methylamino)-1,5-dihydro-2H-pyrano[3,4-c]isoquinolin-6(4H)-one (Vp) and phenyl (3-chloro-4,5-difluorophenyl)carbamate (VId). The enantiomers were subsequently separated by preparative SFC: Method isocratic, Mobile phase 2-propanol: CO₂ —40:60. Column: Chiralpak IA 30×250 mm, 5μ, flow rate: 60 g/min.

Enantiomer I (Compound 107): LCMS: m/z found 438.2/440.2 [M+H]⁺, RT=4.40 min, (Method A); ¹H NMR (400 MHz, DMSO-d₆): δ 11.57 (br s, 1H), 8.70 (s, 1H), 7.90-7.87 (m, 1H), 7.75-7.66 (m, 3H), 7.56-7.52 (m, 1H), 5.43-5.42 (m, 1H), 4.58 (d, 1H), 4.43 (d, 1H), 4.06 (d, 1H), 3.95-3.91 (m, 1H), 2.80 (s, 3H); Chiral analytical SFC: RT=3.84 min, Column: Chiralpak IA (250×4.6 mm), 5μ, 25% 2-propanol, Flow rate: 3.0 ml/min.

Enantiomer II (Compound 108): LCMS: m/z found 438.2/440.2 [M+H]⁺, RT=4.40 min, (Method A); ¹H NMR (400 MHz, DMSO-d₆): δ 11.57 (br s, 1H), 8.70 (s, 1H), 7.90-7.87 (m, 1H), 7.75-7.66 (m, 3H), 7.56-7.52 (m, 1H), 5.43-5.42 (m, 1H), 4.58 (d, 1H), 4.43 (d, 1H), 4.06 (d, 1H), 3.95-3.91 (m, 1H), 2.80 (s, 3H); Chiral analytical SFC: RT=8.02 min, Column: Chiralpak IA (250×4.6 mm), 5μ, 25% 2-propanol, Flow rate: 3.0 ml/min.

### 3-(3-(Difluoromethyl)-4-fluorophenyl)-1-(8-fluoro-6-oxo-1,4,5,6-tetrahydro-2H-pyrano[3,4-c]isoquinolin-1-yl)-1-methylurea (Compounds 123 and 124)

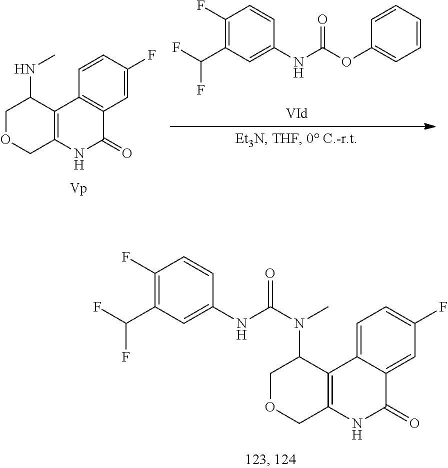

3-(3-(Difluoromethyl)-4-fluorophenyl)-1-(8-fluoro-6-oxo-1,4,5,6-tetrahydro-2H-pyrano[3,4-c]isoquinolin-1-yl)-1-methylurea was synthesized in an analogous manner as described above, from 8-fluoro-1-(methylamino)-1,5-dihydro-2H-pyrano[3,4-c]isoquinolin-6(4H)-one (Vp) and phenyl (3-chloro-4,5-difluorophenyl)carbamate (VIe). The enantiomers were subsequently separated by preparative SFC: Method isocratic, Mobile phase 2-propanol: CO₂— 50:50. Column: Chiralpak IC (30×250 mm), 5μ, flow rate: 100 g/min.

Enantiomer I (Compound 123): LCMS: m/z found 436.3 [M+H]⁺, RT=3.77 min, (Method A); ¹H NMR (400 MHz, DMSO-d₆): δ 11.59 (br s, 1H), 8.62 (br s, 1H), 7.92-7.87 (m, 2H), 7.77-7.65 (m, 2H), 7.59-7.56 (m, 1H), 7.34-7.06 (m, 2H), 5.45-5.44 (m, 1H), 4.58 (d, 1H), 4.43 (d, 1H), 4.05 (d, 1H), 3.95-3.91 (m, 1H), 2.81 (s, 3H); Chiral analytical SFC: RT=0.98 min, Column CHIRALPAK IG-3 (4.6×150 mm) 3 μm, 40% Methanol, Flow rate: 3.0 g/min.

Enantiomer II (Compound 124): LCMS: m/z found 436.2 [M+H]⁺, RT=3.77 min, (Method A); ¹H NMR (400 MHz, DMSO-d₆): δ 11.59 (br s, 1H), 8.62 (br s, 1H), 7.92-7.87 (m, 2H), 7.77-7.65 (m, 2H), 7.59-7.56 (m, 1H), 7.34-7.06 (m, 2H), 5.45-5.44 (m, 1H), 4.58 (d, 1H), 4.43 (d, 1H), 4.05 (d, 1H), 3.95-3.91 (m, 1H), 2.81 (s, 3H); Chiral analytical SFC: RT=7.09 min, Column CHIRALPAK IG-3 (4.6×150 mm) 3 μm, 40% Methanol, Flow rate: 3.0 g/min.

### 8,9-Difluoro-4,5-dihydropyrano[3,4-c]isoquinoline-1,6-dione (IVi)

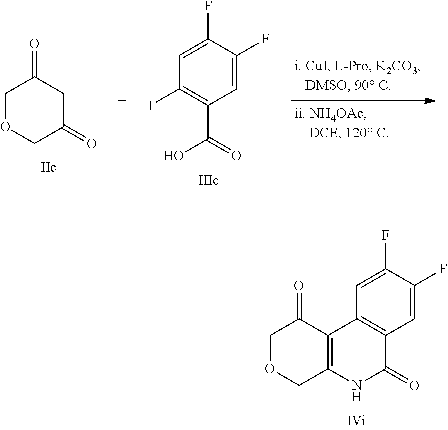 [SMILES: O=C1COCC(=O)C1 ; O=C(O)c1cc(F)c(F)cc1I ; O=C1COCc2[nH]c(=O)c3cc(F)c(F)cc3c21]

Step i: 4,5-Difluoro-2-iodo-benzoic acid (IIIc, 7.50 g, 26.4 mmol), tetrahydropyran-3,5-dione (IIc, 7.53 g, 66.0 mmol), copper (I) iodide (0.50 g, 2.64 mmol), L-Proline (0.61 g, 5.28 mmol), and potassium dicarbonate (21.3 g, 92.43 mmol) were combined in a 250 mL round-bottom flask, which was then evacuated and back-filled with nitrogen. Anhydrous DMSO (90 mL) was added and the reaction mixture was purged with nitrogen, and stirred under a nitrogen atmosphere at room temperature for 10 min, then at 90° C. (preheated bath temperature) for 4 hours. LCMS analysis indicated almost complete conversion of starting material acid to product (<4% left, DAD integration). The reaction mixture was cooled to room temperature, diluted slowly with water until homogeneous, and then acidified with 2 M aqueous HCl to pH<2 at 0° C., and extracted with ethyl acetate (3×400 mL). The combined organic extracts were washed with 5% brine 3 times and with saturated brine once, dried on sodium sulfate, and the solvent was evaporated under vacuum to a residue, which was further dried by azeotropic evaporation with toluene (50 mL), and then on high vacuum overnight, to provide crude 8,9-difluoro-4H-pyrano[3,4-c]isochromene-1,6-dione, which was used in the next step without further purification. ¹H NMR (400 MHz, DMSO-d₆) δ 8.72 (ddd, 1H), 8.25 (ddd, 1H), 4.82 (s, 2H), 4.35 (d, 2H).

Step ii: The crude 8,9-difluoro-4H-pyrano[3,4-c]isochromene-1,6-dione obtained in the step above and ammonium acetate (10.2 g, 132.1 mmol) were stirred in 1,2-dichloroethane (150 mL) at 120° C., in a sealed tube for 5 h. The volatiles were evaporated under vacuum, and the residue was suspended in water and stirred for 15 min, then the product was collected by filtration, washed with water, followed by methanol, and then by diethyl ether, and dried on high vacuum overnight to provide 8,9-difluoro-4,5-dihydropyrano[3,4-c]isoquinoline-1,6-dione (4.53 g, 68%). 252.2 [M+H]⁺; RT=0.74 min (Method B); ¹H NMR (400 MHz, DMSO-d₆) δ 12.33 (s, 1H), 8.90 (dd, 1H), 8.08 (dd, 1H), 4.77 (s, 2H), 4.27 (s, 2H).

### 8,9-Difluoro-1-(methylamino)-1,2,4,5-tetrahydropyrano[3,4-c]isoquinolin-6-one (Vs)

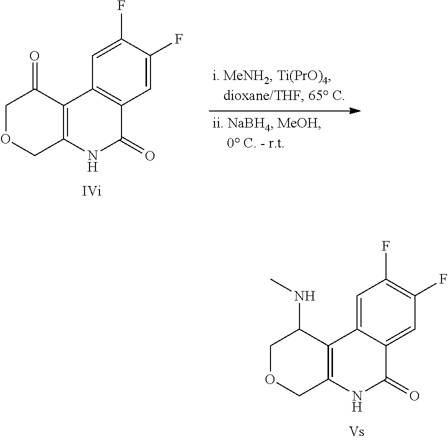 [SMILES: O=C1COCc2[nH]c(=O)c3cc(F)c(F)cc3c21 ; CNC1COCc2[nH]c(=O)c3cc(F)c(F)cc3c21]

8,9-Difluoro-1-(methylamino)-1,2,4,5-tetrahydropyrano[3,4-c]isoquinolin-6-one was synthesized in an analogous manner as described above for Vp, in 87% yield, from 8,9-difluoro-4,5-dihydropyrano[3,4-c]isoquinoline-1,6-dione (IVi) and methylamine. LCMS m/z found 267.1 [M+H]⁺; RT=0.45 min (Method B); ¹H NMR (400 MHz, CDCl₃) δ 11.40 (s, 1H), 8.16 (dd, 1H), 7.54 (dd, 1H), 4.66 (d, 1H), 4.56 (d, 1H), 4.43 (d, 1H), 3.63 (dd, 1H), 3.49 (d, 1H), 2.61 (s, 3H).

### 3-(3-Chloro-4-fluorophenyl)-1-(8,9-difluoro-6-oxo-1,4,5,6-tetrahydro-2H-pyrano[3,4-c]isoquinolin-1-yl)-1-methylurea (Compound 24/Compound 71/Compound 72)

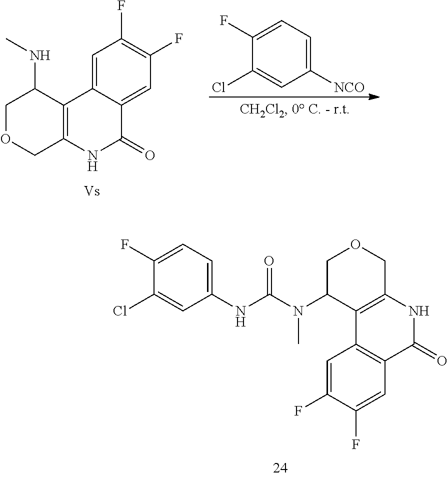

2-Chloro-1-fluoro-4-isocyanato-benzene (32.8 μL, 0.25 mmol) in 0.5 mL of dichloromethane was added slowly to a stirred mixture of 8,9-difluoro-1-(methylamino)-1,2,4,5-tetrahydropyrano[3,4-c]isoquinolin-6-one (Vs, 83.4 mg, 0.31 mmol) in 5 mL of dichloromethane at 0° C. The reaction was stirred for 1.5 hours while allowing it to warm to room temperature. MeOH (1.5 mL) was added and after 15 min the solvent was evaporated under vacuum to near dryness. The product was triturated with methanol and was collected by filtration, washed with methanol, followed by 1:1 methanol/dichloromethane, and then with hexane, and dried in high vacuum to provide racemic 3-(3-chloro-4-fluorophenyl)-1-(8,9-difluoro-6-oxo-1,4,5,6-tetrahydro-2H-pyrano[3,4-c]isoquinolin-1-yl)-1-methylurea (118.0 mg, 86.0%). LCMS: m/z found 438.1/440.2 [M+H]⁺; RT=4.24 min, (Method A); ¹H NMR (400 MHz, DMSO-d₆) δ 11.67 (s, 1H), 8.60 (s, 1H), 8.11 (dd, 1H), 7.84 (dd, 1H), 7.56-7.43 (m, 2H), 7.34 (t, 1H), 5.41 (s, 1H), 4.59 (d, 1H), 4.47-4.37 (m, 1H), 4.10-4.02 (m, 1H), 3.93 (dd, 1H), 2.82 (s, 3H). The enantiomers were subsequently separated by preparative SFC: Method isocratic, Mobile phase MeOH: CO₂—40:60. Column: CHIRALPAK AD (30×150 mm) 5 m; Total flow: 90 g/min.

(R)-3-(3-Chloro-4-fluorophenyl)-1-(8,9-difluoro-6-oxo-1,4,5,6-tetrahydro-2H-pyrano[3,4-c]isoquinolin-1-yl)-1-methylurea: Enantiomer I (Compound 71). LCMS m/z found 438.2/440.2 [M+H]⁺; RT=4.26 min (Method A); Chiral analytical SFC: RT=4.06 min, Column: CHIRALPAK IC-3 (4.6×150 mm) 3 m; 40% Methanol; Total flow: 3 g/min.

(S)-3-(3-Chloro-4-fluorophenyl)-1-(8,9-difluoro-6-oxo-1,4,5,6-tetrahydro-2H-pyrano[3,4-c]isoquinolin-1-yl)-1-methylurea: Enantiomer II (Compound 72). LCMS m/z found 438.2/440.2 [M+H]⁺; RT=4.26 min (Method A); Chiral analytical SFC: RT=6.55 min, Column: CHIRALPAK IC-3 (4.6×150 mm) 3 m; 20% Methanol; Total flow: 3 g/min.

Compound 72 was also prepared independently as described below and according to the general Scheme 3.

### (S)-8,9-Difluoro-1-(((R)-1-(4-methoxyphenyl)ethyl)amino)-1,5-dihydro-2H-pyrano[3,4-c]isoquinolin-6(4H)-one (Xb)

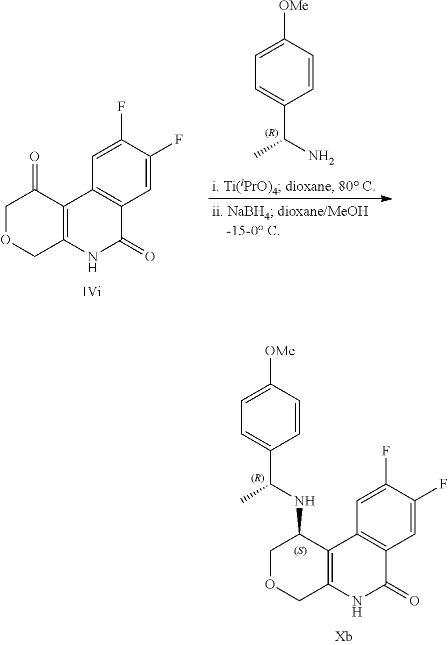

Diastereomerically pure (S)-8,9-difluoro-1-(((R)-1-(4-methoxyphenyl)ethyl)amino)-1,5-dihydro-2H-pyrano[3,4-c]isoquinolin-6(4H)-one was synthesized in an analogous manner as described above for Xa, in 69% yield, starting from 8,9-difluoro-4,5-dihydropyrano[3,4-c]isoquinoline-1,6-dione (IVi). LCMS m/z found 387.27 [M+H]⁺; RT=0.60 min (Method B); ¹H NMR (400 MHz, CDCl₃) δ 11.29 (s, 1H), 8.14 (dd, 1H), 7.67 (dd, 1H), 7.31-7.27 (m, 2H), 6.90-6.79 (m, 2H), 4.61 (d, 1H), 4.55-4.46 (m, 1H), 4.23-4.15 (m, 1H), 4.08 (q, 1H), 3.84-3.76 (m, 1H), 3.78 (s, 3H), 3.54 (dd, 1H), 1.47 (d, 3H).

The stereochemistry of the newly generated chiral α-center was shown to be (S)-based on X-ray crystallographic analysis of Compound 72 (see elsewhere herein).

### (S)-1-(3-Chloro-4-fluorophenyl)-3-(8,9-difluoro-6-oxo-1,4,5,6-tetrahydro-2H-pyrano[3,4-c]isoquinolin-1-yl)urea (Compound 68)

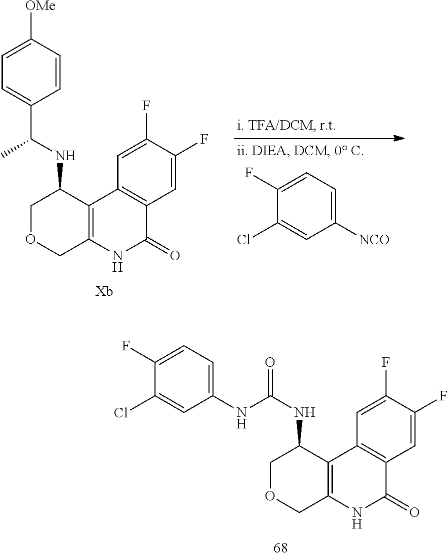

Enantiomerically pure (S)-1-(3-chloro-4-fluorophenyl)-3-(8,9-difluoro-6-oxo-1,4,5,6-tetrahydro-2H-pyrano[3,4-c]isoquinolin-1-yl)urea was synthesized in an analogous manner as described above for Compound 41, from optically pure (S)-8,9-difluoro-1-(((R)-1-(4-methoxyphenyl)ethyl)amino)-1,5-dihydro-2H-pyrano[3,4-c]isoquinolin-6(4H)-one (Xb). LCMS m/z 424.2 [M+H]⁺; RT=4.20 min (Method A); ¹H NMR (400 MHz, DMSO-d6) δ 11.57 (s, 1H), 8.62 (s, 1H), 8.09 (dd, 1H), 7.78 (dd, 1H), 7.68 (dd, 1H), 7.29 (t, 1H), 7.22 (ddd, 1H), 6.80 (d, 1H), 4.88 (d, 1H), 4.55-4.40 (m, 2H), 4.00 (dd, 1H), 3.84 (dd, 1H).

### (S)-1-(3-Cyano-4-fluorophenyl)-3-(8,9-difluoro-6-oxo-1,4,5,6-tetrahydro-2H-pyrano[3,4-c]isoquinolin-1-yl)urea(Compound 135)

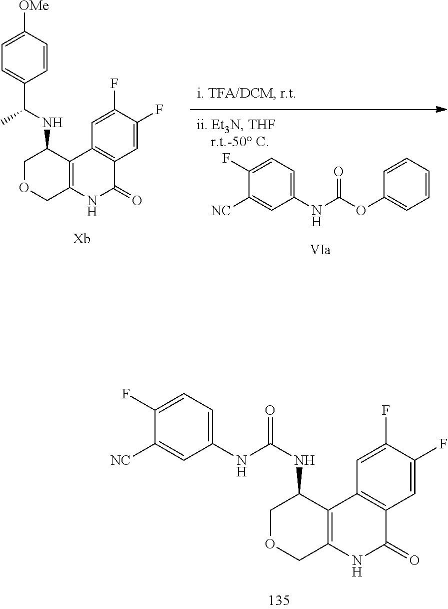 [SMILES: COc1ccc([C@@H](C)N[C@@H]2COCc3[nH]c(=O)c4cc(F)c(F)cc4c32)cc1 ; N#Cc1cc(NC(=O)Oc2ccccc2)ccc1F ; N#Cc1cc(NC(=O)N[C@@H]2COCc3[nH]c(=O)c4cc(F)c(F)cc4c32)ccc1F]

Enantiomerically pure (S)-1-(3-cyano-4-fluorophenyl)-3-(8,9-difluoro-6-oxo-1,4,5,6-tetrahydro-2H-pyrano[3,4-c]isoquinolin-1-yl)urea was synthesized in an analogous manner as described for Compounds 41 and 70, from optically pure (S)-8,9-difluoro-1-(((R)-1-(4-methoxyphenyl)ethyl)amino)-1,5-dihydro-2H-pyrano[3,4-c]isoquinolin-6(4H)-one (Xb) and phenyl N-(3-cyano-4-fluoro-phenyl)carbamate (VIa). LCMS m/z 415.3 [M+H]⁺; RT=3.67 min (Method A); ¹H NMR (400 MHz, DMSO-d₆) δ 11.58 (s, 1H), 8.76 (s, 1H), 8.09 (dd, 1H), 7.94 (dd, 1H), 7.73-7.62 (m, 2H), 7.43 (t, 1H), 6.91 (d, 1H), 4.88 (d, 1H), 4.55-4.41 (m, 2H), 4.01 (d, 1H), 3.84 (dd, 1H).

### (S)-8,9-Difluoro-1-(((R)-1-(4-methoxyphenyl)ethyl)(methyl)amino)-1,5-dihydro-2H-pyrano[3,4-c]isoquinolin-6(4H)-one (XIc)

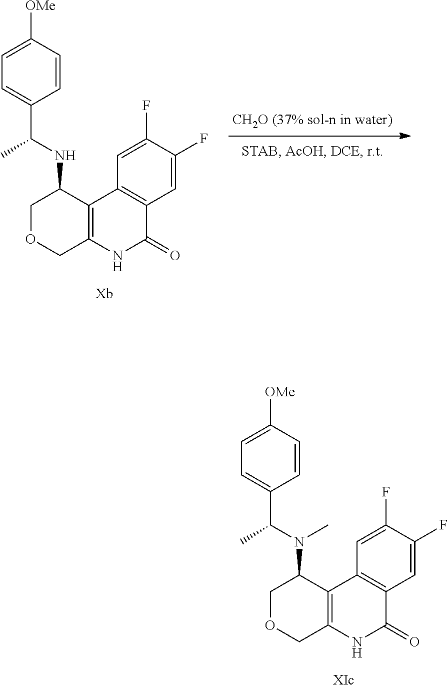 [SMILES: COc1ccc([C@@H](C)N[C@@H]2COCc3[nH]c(=O)c4cc(F)c(F)cc4c32)cc1 ; COc1ccc([C@@H](C)N(C)[C@@H]2COCc3[nH]c(=O)c4cc(F)c(F)cc4c32)cc1]

Diastereomerically pure (S)-8,9-difluoro-1-(((R)-1-(4-methoxyphenyl)ethyl)(methyl)amino)-1,5-dihydro-2H-pyrano[3,4-c]isoquinolin-6(4H)-one was synthesized in an analogous manner as described above for XIa, in 82% yield, starting from (S)-8,9-difluoro-1-(((R)-1-(4-methoxyphenyl)ethyl)amino)-1,5-dihydro-2H-pyrano[3,4-c]isoquinolin-6(4H)-one (Xb). LCMS m/z found 401.3 [M+H]⁺; RT=2.24 min (Method A); H NMR (400 MHz, CDCl₃) δ 12.01 (s, 1H), 8.14 (dd, 1H), 8.03 (dd, 1H), 7.22-7.10 (m, 2H), 6.85-6.74 (m, 2H), 4.70 (d, 1H), 4.53 (dd, 1H), 4.45 (d, 1H), 4.19-4.06 (m, 1H), 3.90 (q, 1H), 3.78 (s, 3H), 3.63 (dd, 1H), 2.14 (s, 3H), 1.51 (d, 3H).

### (S)-8,9-Difluoro-1-(methylamino)-1,5-dihydro-2H-pyrano[3,4-c]isoquinolin-6(4H)-one (Vs); enantiomerically pure

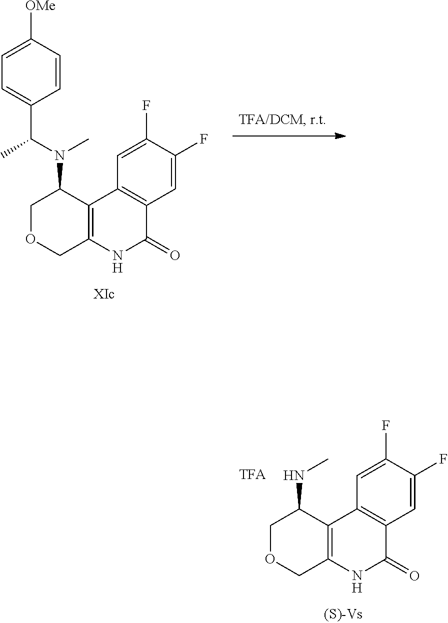 [SMILES: COc1ccc([C@@H](C)N(C)[C@@H]2COCc3[nH]c(=O)c4cc(F)c(F)cc4c32)cc1 ; CN[C@@H]1COCc2[nH]c(=O)c3cc(F)c(F)cc3c21]

(S)-8,9-Difluoro-1-(((R)-1-(4-methoxyphenyl)ethyl)(methyl)amino)-1,5-dihydro-2H-pyrano[3,4-c]isoquinolin-6(4H)-one (XIc, 1.93 g, 4.82 mmol) was stirred overnight with trifluoroacetic acid (20 mL, 175.4 mmol) in dichloromethane (20.0 mL) at room temperature, under nitrogen. The reaction mixture was then treated with 40 mL of MeOH and the mixture stirred for 20 min, when the deep purple, opaque mixture transitioned to a yellow, transparent solution. The volatiles were evaporated, and the residue was dried further by azeotropic evaporation with a 1:1 v/v methanol/toluene mixture, then once with toluene. Trituration with diethyl ether for 15 min generated a precipitate that was collected by filtration, washed with diethyl ether, and dried under high vacuum to provide (S)-8,9-difluoro-1-(methylamino)-1,5-dihydro-2H-pyrano[3,4-c]isoquinolin-6(4H)-one as a single enantiomer, mono-TFA salt (1.67 g, 91%). LCMS found m/z 267.2 [M+H]⁺; RT=0.47 min (Method B); ¹H NMR (400 MHz, Methanol-d₄) δ 8.17 (dd, 1H), 7.83 (dd, 1H), 4.89 (s, 1H), 4.76-4.60 (m, 2H), 4.58 (s, 1H), 4.51 (dd, 1H), 3.98 (dd, 1H), 2.86 (s, 3H). A portion of the TFA salt of (S)-Vs, obtained as above, was partitioned between ethyl acetate and saturated sodium bicarbonate. The aqueous phase was further extracted with ethyl acetate, ensuring a pH >8.5 after the final extraction, and the combined organic extracts were dried over sodium sulfate, filtered, the solvent was evaporated under reduced pressure and the solid residue was further dried under high vacuum to afford enantiomerically pure (S)-8,9-difluoro-1-(methylamino)-1,5-dihydro-2H-pyrano[3,4-c]isoquinolin-6(4H)-one (Vs) as a free base. ¹H NMR (400 MHz, DMSO-d₆) δ 11.40 (br s, 1H), 8.03 (dd, 1H), 7.73 (dd, 1H), 4.41 (d, 1H), 4.34 (d, 1H), 4.22 (dd, 1H), 3.59-3.51 (m, 1H), 3.33 (s, 1H), 2.39 (s, 3H), 1.90 (br s, 1H).

### (S)-3-(3-Chloro-4-fluorophenyl)-1-(8,9-difluoro-6-oxo-1,4,5,6-tetrahydro-2H-pyrano[3,4-c]isoquinolin-1-yl)-1-methylurea: Enantiomer II (Compound 72)

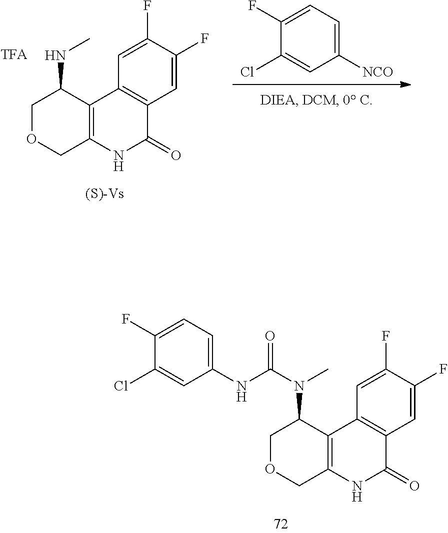

Enantiomerically pure (S)-3-(3-chloro-4-fluorophenyl)-1-(8,9-difluoro-6-oxo-1,4,5,6-tetrahydro-2H-pyrano[3,4-c]isoquinolin-1-yl)-1-methylurea: Enantiomer II was synthesized in an analogous manner as described above for Compound 41, in 75% yield, starting from enantiomerically pure (S)-8,9-difluoro-1-(methylamino)-1,5-dihydro-2H-pyrano[3,4-c]isoquinolin-6(4H)-one mono-TFA salt (Vs). LCMS m/z found 438.2/440.2 [M+H]⁺; RT=4.26 min (Method A); ¹H NMR (400 MHz, DMSO-d₆) δ 11.66 (s, 1H), 8.60 (s, 1H), 8.10 (dd, 1H), 7.84 (dt, 1H), 7.57-7.42 (m, 2H), 7.34 (t, 1H), 5.41 (d, 1H), 4.59 (d, 1H), 4.42 (dd, 1H), 4.06 (dd, 1H), 3.93 (dd, 1H), 2.82 (s, 3H); Chiral analytical SFC: RT=4.83 min, Column: OD-10 analytical; 20% Methanol; Total flow: 3 g/min; ee=99.5%.

### X-Ray Structure Determination of Compound 72

Crystals of Compound 72 were grown by vapor diffusion, using 1:3 v/v methanol:dichloromethane as the solvent and 1:2 v/v diethyl ether:hexanes as the anti-solvent. Compound 72 (Molecular formula: C₂₀H₁₅ClF₃N₃O₃), crystallizes in the orthorhombic space group P2₁2₁2₁ (systematic absences h00: h=odd, 0k0: k=odd, and 00l: 1=odd) with a=7.53710(10)Å, b=8.59410(10)Å, c=27.5971(2)Å, V=1787.59(3)Å³, Z=4, and d_{calc}=1.627 g/cm³. X-ray intensity data were collected on a Rigaku XtaLAB Synergy-S diffractometer equipped with an HPC area detector (HyPix-6000HE) and employing confocal multilayer optic-monochromated Cu-Kα radiation (λ=1.54184 Å) at a temperature of 100K. Preliminary indexing was performed from a series of sixty 0.5° rotation frames with exposures of 0.25 second for θ=±47.2° and 1 second for θ=107.75°. A total of 4658 frames (41 runs) were collected employing co scans with a crystal to detector distance of 34.0 mm, rotation widths of 0.5° and exposures of 0.05 second for θ=±47.2° and 0.1 second for θ=107.75°.

Rotation frames were integrated using CrysAlisPro (CrysAlisPro 1.171.40.53: Rigaku Oxford Diffraction, Rigaku Corporation, Oxford, U K, 2019), producing a listing of unaveraged F² and σ(F²) values. A total of 30097 reflections were measured over the ranges 6.406≤2θ≤148.832°, −9≤h≤9, −10≤k≤10, −27≤1≤34 yielding 3668 unique reflections (Rᵢₙₜ=0.0517). The intensity data were corrected for Lorentz and polarization effects and for absorption using SCALE3 ABSPACK (SCALE3 ABSPACK v1.0.7: an Oxford Diffraction program; Oxford Diffraction Ltd: Abingdon, U K, 2005) (minimum and maximum transmission 0.6358, 1.0000). The structure was solved by direct methods—SHELXT (SHELXT v2014/4: Sheldrick, G. M., Acta Cryst., A, 71, 3-8 (2015)). Refinement was by full-matrix least squares based on F² using SHELXL-2018 (SHELXL-2018/3: Sheldrick, G. M., Acta Cryst., A, 71, 3-8 (2015)). All reflections were used during refinement. The weighting scheme used was w=1/[σ²(Fₒ²)+(0.0398P)²+0.4843P] where P=(Fₒ²+2F_{c}²)/3. Non-hydrogen atoms were refined anisotropically and hydrogen atoms were refined using a riding model. Refinement converged to R1=0.0255 and wR2=0.0666 for 3642 observed reflections for which F>4σ(F) and R1=0.0257 and wR2=0.0667 and GOF=1.028 for all 3668 unique, non-zero reflections and 272 variables. The maximum Δ/σ in the final cycle of least squares was 0.001 and the two most prominent peaks in the final difference Fourier were +0.18 and −0.31 e/Å³.

Table 1. lists cell information, data collection parameters, and refinement data for Compound 72.

TABLE 1
Summary of Structure Determination of Compound 72

| Empirical formula | C₂₀H₁₅ClF₃N₃O₃ |
|---|---|
| Formula weight | 437.80 |
| Diffractometer | Rigaku XtaLAB Synergy-S |
| | (HyPix-6000HE) |
| Temperature/K | 100 |
| Crystal system | orthorhombic |
| Space group | P2₁2₁2₁ |
| a | 7.53710(10) Å |
| b | 8.59410(10) Å |
| c | 27.5971(2) Å |
| Volume | 1787.59(3) Å³ |
| Z | 4 |
| d_{calc} | 1.627 g/cm³ |
| μ | 2.455 mm⁻¹ |
| F(000) | 896.0 |
| Crystal size, mm | 0.29 × 0.16 × 0.08 |
| 2θ range for data collection | 6.406-148.832° |
| Index ranges | −9 ≤ h ≤ 9, −10 ≤ k ≤ |
| | 10, −27 ≤ l ≤ 34 |
| Reflections collected | 30097 |
| Independent reflections | 3668[R(int) = 0.0517] |
| Data/restraints/parameters | 3668/0/272 |
| Goodness-of-fit on F² | 1.028 |
| Final R indexes [I >= 2σ (I)] | R₁ = 0.0255, wR₂ = 0.0666 |
| Final R indexes [all data] | R₁ = 0.0257, wR₂ = 0.0667 |
| Largest diff. peak/hole | 0.18/−0.31 eÅ⁻³ |
| Flack parameter | 0.005(6) |

Final positional and equivalent isotropic thermal parameters for Compound 72 are given in Table 2.

TABLE 2
Refined Positional Parameters for Compound 72

| Atom | x | y | z | U(eq) |
|---|---|---|---|---|
| Cl1 | 0.54718(6) | 0.99723(6) | 0.31799(2) | 0.02003(12) |
| F1 | 0.19703(17) | 1.17189(13) | 0.65035(4) | 0.0237(3) |
| F2 | 0.28355(18) | 1.09655(14) | 0.55966(4) | 0.0219(3) |
| F3 | 0.37770(18) | 0.77377(15) | 0.25306(4) | 0.0241(3) |
| O1 | 0.41654(19) | 0.71994(18) | 0.75288(5) | 0.0206(3) |
| O2 | 0.6208(2) | 0.29469(16) | 0.59308(5) | 0.0177(3) |
| O3 | 0.58862(19) | 0.73504(17) | 0.47876(5) | 0.0177(3) |
| N1 | 0.5005(2) | 0.54255(19) | 0.69614(5) | 0.0141(3) |
| N2 | 0.4538(2) | 0.54977(18) | 0.52504(5) | 0.0137(3) |
| N3 | 0.3861(2) | 0.5726(2) | 0.44343(5) | 0.0152(3) |
| C1 | 0.4381(2) | 0.6860(2) | 0.70974(6) | 0.0147(4) |
| C2 | 0.3979(2) | 0.7924(2) | 0.66959(7) | 0.0139(4) |
| C3 | 0.3156(2) | 0.9352(2) | 0.67993(7) | 0.0162(4) |
| C4 | 0.2793(3) | 1.0346(2) | 0.64268(7) | 0.0169(4) |
| C5 | 0.3271(3) | 0.9962(2) | 0.59535(6) | 0.0161(4) |
| C6 | 0.4094(3) | 0.8599(2) | 0.58428(7) | 0.0144(4) |
| C7 | 0.4438(2) | 0.7523(2) | 0.62163(6) | 0.0125(3) |
| C8 | 0.5214(2) | 0.6022(2) | 0.61194(6) | 0.0126(4) |
| C9 | 0.5417(2) | 0.5004(2) | 0.64896(6) | 0.0132(3) |
| C10 | 0.6132(3) | 0.3387(2) | 0.64287(7) | 0.0158(4) |
| C11 | 0.7055(3) | 0.4140(2) | 0.56535(7) | 0.0175(4) |
| C12 | 0.5926(3) | 0.5614(2) | 0.56234(6) | 0.0133(4) |
| C13 | 0.3115(3) | 0.4371(2) | 0.53117(7) | 0.0180(4) |
| C14 | 0.4833(3) | 0.6258(2) | 0.48232(7) | 0.0137(4) |
| C15 | 0.3941(3) | 0.6286(2) | 0.39528(6) | 0.0134(4) |
| C16 | 0.4676(3) | 0.7715(2) | 0.38255(7) | 0.0145(3) |
| C17 | 0.4601(3) | 0.8183(2) | 0.33442(7) | 0.0155(4) |
| C18 | 0.3847(3) | 0.7238(2) | 0.29946(7) | 0.0172(4) |
| C19 | 0.3166(3) | 0.5803(3) | 0.31153(7) | 0.0177(4) |
| C20 | 0.3204(3) | 0.5332(2) | 0.35951(7) | 0.0158(4) |

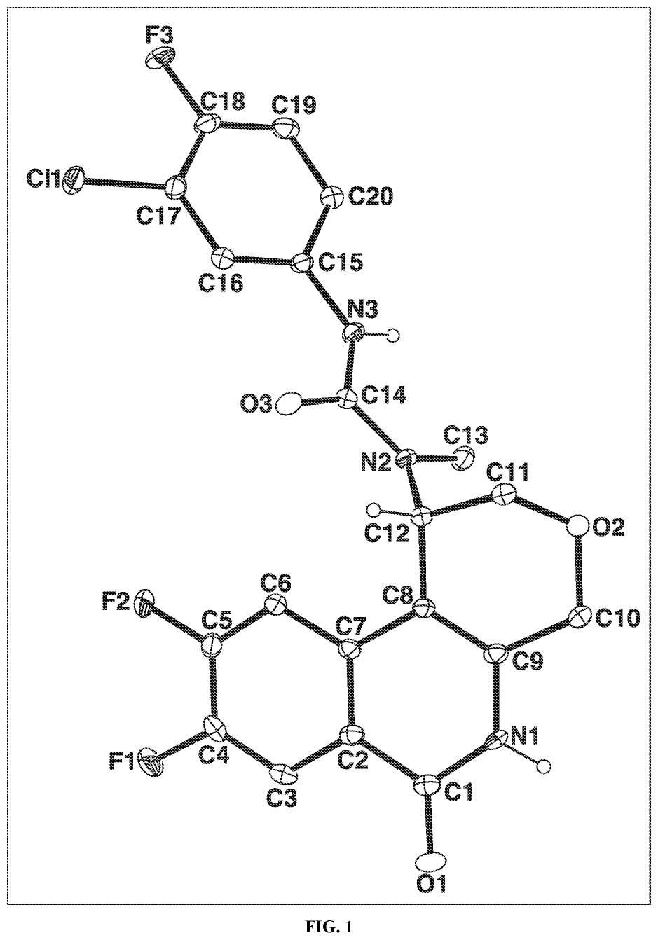

The ORTEP representation of Compound 72 defining the absolute configuration of Compound 72 as (S)- (and consequently the absolute configuration of the α-methyl substituent of the major diastereoisomer of XIc as (S)-) is shown in FIG. 1.

### 8,9-Difluoro-6-methoxy-N—((R)-1-(4-methoxyphenyl)ethyl)-N-methyl-1,4-dihydro-2H-pyrano[3,4-c]isoquinolin-1-amine (XIe) and 8,9-difluoro-1-(((R)-1-(4-methoxyphenyl)ethyl)(methyl)amino)-5-methyl-1,5-dihydro-2H-pyrano[3,4-c]isoquinolin-6(41H)-one (XIf)

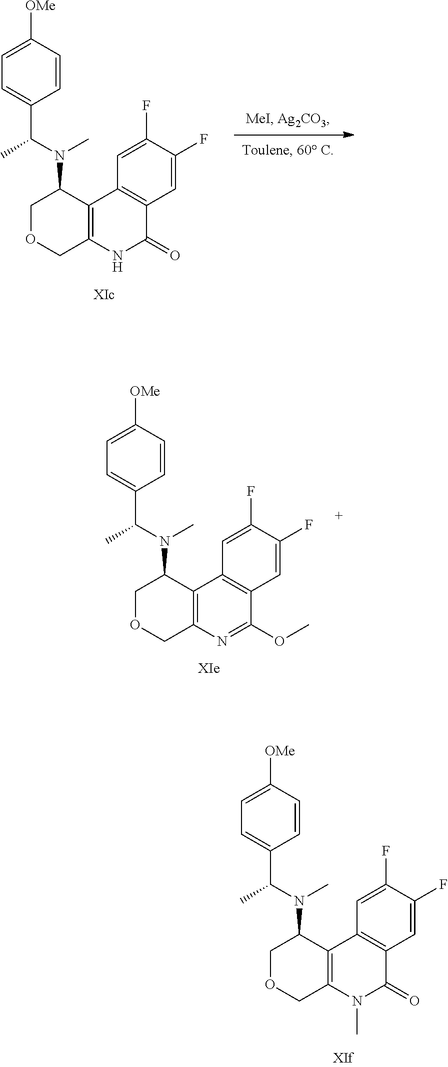 [SMILES: COc1ccc([C@@H](C)N(C)[C@@H]2COCc3[nH]c(=O)c4cc(F)c(F)cc4c32)cc1 ; COc1ccc([C@@H](C)N(C)[C@@H]2COCc3nc(OC)c4cc(F)c(F)cc4c32)cc1 ; COc1ccc([C@@H](C)N(C)[C@@H]2COCc3c2c2cc(F)c(F)cc2c(=O)n3C)cc1]

To a stirred solution of 1 g (2.4 mmol, 1.0 eq) of diastereomerically pure (S)-8,9-difluoro-1-(((R)-1-(4-methoxyphenyl)ethyl)(methyl)amino)-1,5-dihydro-2H-pyrano[3,4-c]isoquinolin-6(4H)-one (XIc) in 10 mL of toluene at room temperature was added 1.3 g (4.9 mmol, 2.0 eq) of silver carbonate and 0.2 mL (4.9 mmol, 2.0 eq) of methyl iodide. The reaction mixture was stirred at 60° C. for 16 h. Upon cooling, the reaction mixture was filtered through a pad of Celite and the Celite bed was washed with EtOAc (100 mL). The combined filtrate was concentrated under reduced pressure and the crude product mixture was purified by column chromatography (using 10%-20% of ethyl acetate in petroleum ether as a linear gradient) to afford 400 mg of (S)-8,9-difluoro-6-methoxy-N—((R)-1-(4-methoxyphenyl)ethyl)-N-methyl-1,4-dihydro-2H-pyrano[3,4-c]isoquinolin-1-amine (XIe, 0.96 mmol, 38%) and 300 mg of (S)-8,9-difluoro-1-(((R)-1-(4-methoxyphenyl)ethyl)(methyl)amino)-5-methyl-1,5-dihydro-2H-pyrano[3,4-c]isoquinolin-6(4H)-one (XIf, 0.72 mmol, 30%) as pale yellow solids. XIe: LCMS: m/z found 415.31 [M+H]⁺; RT=1.76 min, (Method D); ¹HNMR (400 MHz, DMSO-d6): δ 8.23 (m, 1H), 8.05 (m, 1H), 7.16 (d, 2H), 6.83 (d, 2H), 4.69 (d, 1H), 4.55 (d, 1H), 4.37 (d, 2H), 4.01 (s, 3H), 3.94 (m, 1H), 3.70 (s, 3H), 3.64 (m, 1H), 1.94 (s, 3H), 1.46 (d, 3H). XIf: LCMS: m/z found 415.31 [M+H]⁺; RT=1.42 min, (Method D); ¹HNMR (400 MHz, DMSO-d6): δ 8.12-7.99 (m, 2H), 7.14 (d, 2H), 6.81 (d, 2H), 4.84 (d, 1H), 4.51 (d, 1H), 4.29 (d, 1H), 4.16 (s, 1H), 3.98 (m, 1H), 3.75 (s, 3H), 3.53 (m, 1H), 3.40 (s, 3H), 2.25 (s, 3H), 1.46 (d, 3H).

### 8,9-Difluoro-5-methyl-1-(methylamino)-1,5-dihydro-2H-pyrano[3,4-c]isoquinolin-6(4H)-one (Vaaa)

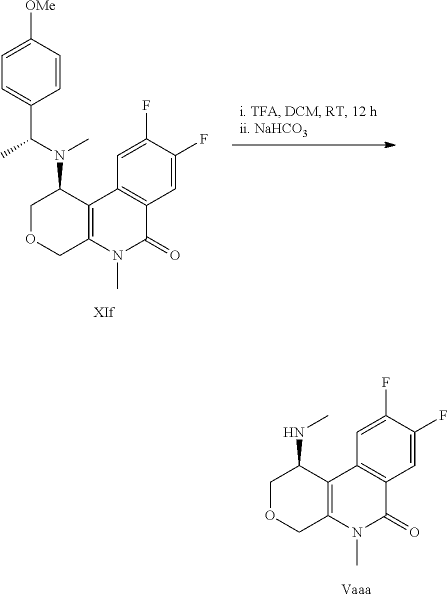 [SMILES: COc1ccc([C@@H](C)N(C)[C@@H]2COCc3c2c2cc(F)c(F)cc2c(=O)n3C)cc1 ; CN[C@@H]1COCc2c1c1cc(F)c(F)cc1c(=O)n2C]

To a stirred solution of 300 mg (0.18 mmol, 1.0 eq) of (S)-8,9-difluoro-1-(((R)-1-(4-methoxyphenyl)ethyl)(methyl)amino)-5-methyl-1,5-dihydro-2H-pyrano[3,4-c]isoquinolin-6(4H)-one (XIf) in 5 mL of DCM was added 0.5 mL of TFA at 0° C. The mixture was allowed to warm to RT and stirred for 12 h. The mixture was quenched with saturated aqueous sodium bicarbonate solution and extracted with EtOAc (2×50 mL). The combined organics were washed with ice-water, dried over sodium sulfate and evaporated to provide 120 mg crude (S)-8,9-difluoro-5-methyl-1-(methylamino)-1,5-dihydro-2H-pyrano[3,4-c]isoquinolin-6(4H)-one, which was directly used for the next step. LCMS: m/z found 281.2 [M+H]⁺; RT=0.84 min, (Method D).

### (S)-1-(8,9-Difluoro-5-methyl-6-oxo-1,4,5,6-tetrahydro-2H-pyrano[3,4-c]isoquinolin-1-yl)-3-(3-(difluoromethyl)-4-fluorophenyl)-1-methylurea (Compound 233)

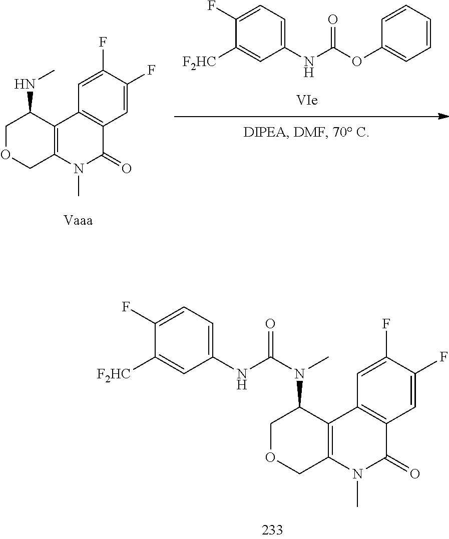 [SMILES: CN[C@@H]1COCc2c1c1cc(F)c(F)cc1c(=O)n2C ; O=C(Nc1ccc(F)c(C(F)F)c1)Oc1ccccc1 ; CN(C(=O)Nc1ccc(F)c(C(F)F)c1)[C@@H]1COCc2c1c1cc(F)c(F)cc1c(=O)n2C]

To a stirred solution of 80 mg (0.28 mmol, 1.0 eq) of crude (S)-8,9-difluoro-5-methyl-1-(methylamino)-1,5-dihydro-2H-pyrano[3,4-c]isoquinolin-6(4H)-one (Vaaa) in 2 mL of DMF at room temperature were added 0.12 mL (0.86 mmol, 3.0 eq) of DIPEA followed by 80 mg (0.28 mmol, 1.0 eq) of phenyl (3-(difluoromethyl)-4-fluorophenyl)carbamate (VIe) under inert atmosphere. The reaction mixture was heated to 70° C. with stirring for 1 h. After reaction completion, the reaction mixture was diluted with ice-cold water (40 mL). The resulting precipitate was filtered, washed with water (10 mL) and dried under vacuum. The obtained crude solid product was purified by flash chromatography (Silicagel, MeOH/DCM 5 —10%) to provide 30 mg (0.06 mmol, 22%) of (S)-1-(8,9-difluoro-5-methyl-6-oxo-1,4,5,6-tetrahydro-2H-pyrano[3,4-c]isoquinolin-1-yl)-3-(3-(difluoromethyl)-4-fluorophenyl)-1-methylurea as an off white solid. LCMS m/z found 468.1 [M+H]⁺; RT=4.35 min, (Method A); ¹H (400 MHz, DMSO-d6): δ 8.64 (s, 1H), 8.20 (m, 1H), 7.88-7.87 (m, 1H), 7.74-7.66 (m, 1H), 7.52-7.50 (m, 1H), 7.34-7.07 (m, 2H), 5.47 (s, 1H), 4.96 (d, 1H), 4.64 (d, 1H), 4.08 (d, 1H), 3.90 (m, 1H), 3.44 (s, 3H), 2.82 (s, 3H). Chiral analytical SFC: RT=3.29 min, Column: ChiralPakl AD-3 (4.6×150 mm) 3 μm, 30% (0.5% DEA in methanol), Flow rate: 3 g/min.

### (S)-3-(3-Chloro-4-fluorophenyl)-1-(8,9-difluoro-5-methyl-6-oxo-1,4,5,6-tetrahydro-2H-pyrano[3,4-c]isoquinolin-1-yl)-1-methylurea (Compound 243)

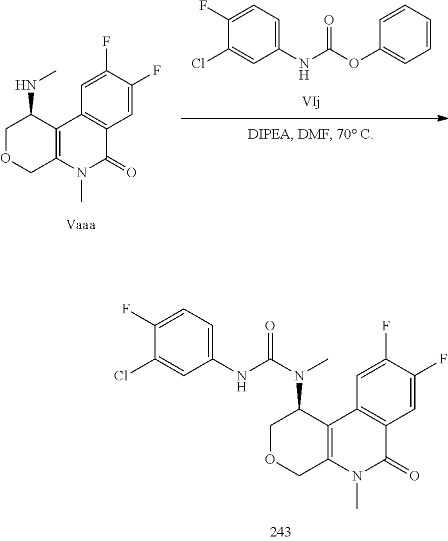 [SMILES: CN[C@@H]1COCc2c1c1cc(F)c(F)cc1c(=O)n2C ; O=C(Nc1ccc(F)c(Cl)c1)Oc1ccccc1 ; CN(C(=O)Nc1ccc(F)c(Cl)c1)[C@@H]1COCc2c1c1cc(F)c(F)cc1c(=O)n2C]

(S)-3-(3-chloro-4-fluorophenyl)-1-(8,9-difluoro-5-methyl-6-oxo-1,4,5,6-tetrahydro-2H-pyrano[3,4-c]isoquinolin-1-yl)-1-methylurea was synthesized in an analogous manner as above from of crude (S)-8,9-difluoro-5-methyl-1-(methylamino)-1,5-dihydro-2H-pyrano[3,4-c]isoquinolin-6(4H)-one (Vaaa) and phenyl (3-chloro-4-fluorophenyl)carbamate (VIj). LCMS m/z found 452.1/454.0 [M+H]⁺; RT=5.89 min, (Method A); ¹H (400 MHz, DMSO-d6): δ 8.59 (s, 1H), 8.20 (m, 1H), 7.88-7.87 (m, 1H), 7.56-7.45 (m, 2H), 7.36 (t, 1H), 5.46 (s, 1H), 4.96 (d, 1H), 4.64 (d, 1H), 4.08 (d, 1H), 3.90 (m, 1H), 3.45 (s, 3H), 2.82 (s, 3H). Chiral analytical SFC: RT=2.34 min, Column: ChiralPak AD-3 (4.6×150 mm) 3 μm, 30% (0.5% DEA in methanol), Flow rate: 3 g/min.

### 8,9-difluoro-6-methoxy-N-methyl-1,4-dihydro-2H-pyrano[3,4-c]isoquinolin-1-amine (V-Bc)

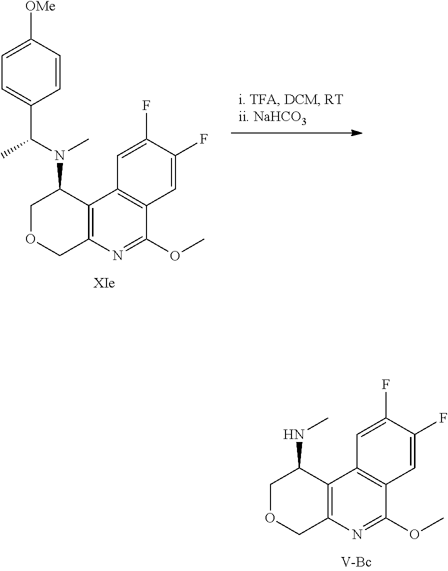 [SMILES: COc1ccc([C@@H](C)N(C)[C@@H]2COCc3nc(OC)c4cc(F)c(F)cc4c32)cc1 ; CN[C@@H]1COCc2nc(OC)c3cc(F)c(F)cc3c21]

To a stirred solution of 400 mg (0.18 mmol, 1.0 eq) (S)-8,9-difluoro-6-methoxy-N—((R)-1-(4-methoxyphenyl)ethyl)-N-methyl-1,4-dihydro-2H-pyrano[3,4-c]isoquinolin-1-amine (XIe) in 5 mL of DCM was added 0.5 mL of TFA at 0° C. The mixture was allowed to warm to RT and stirred for 12 h. The reaction mixture was quenched with saturated aqueous sodium bicarbonate solution and extracted with EtOAc (2×50 mL). The combined organics were washed with ice-water, dried over sodium sulfate and evaporated to dryness to provide 200 mg crude (S)-8,9-difluoro-6-methoxy-N-methyl-1,4-dihydro-2H-pyrano[3,4-c]isoquinolin-1-amine was directly used for the next step. LCMS: m/z found 281.23 [M+H]⁺.

### (S)-1-(8,9-Difluoro-6-methoxy-1,4-dihydro-2H-pyrano[3,4-c]isoquinolin-1-yl)-3-(3-(difluoromethyl)-4-fluorophenyl)-1-methylurea (Compound 234)

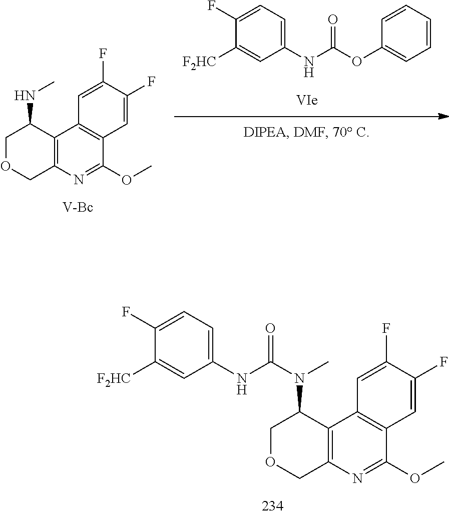 [SMILES: CN[C@@H]1COCc2nc(OC)c3cc(F)c(F)cc3c21 ; O=C(Nc1ccc(F)c(C(F)F)c1)Oc1ccccc1 ; COc1nc2c(c3cc(F)c(F)cc13)[C@H](N(C)C(=O)Nc1ccc(F)c(C(F)F)c1)COC2]

To a stirred solution of 80 mg (0.28 mmol, 1.0 eq) 8,9-difluoro-6-methoxy-N-methyl-1,4-dihydro-2H-pyrano[3,4-c]isoquinolin-1-amine (V-Bc) in 2 mL of DMF at room temperature were added 0.12 mL (0.86 mmol, 3.0 eq) of DIPEA followed by 80 mg (0.28 mmol, 1.0 eq) of phenyl (3-(difluoromethyl)-4-fluorophenyl)carbamate (VIe) under inert atmosphere. The reaction mixture was heated to 70° C. with stirring for 1 h. Upon cooling, the reaction mixture was diluted with ice-cold water (40 mL). The solid precipitated was filtered, washed with water (10 mL) and dried under vacuum. The obtained crude solid product was purified by flash chromatography (Silicagel, MeOH/DCM 5-10%) to provide 30 mg (0.06 mmol, 22%) of (S)-1-(8,9-difluoro-6-methoxy-1,4-dihydro-2H-pyrano[3,4-c]isoquinolin-1-yl)-3-(3-(difluoromethyl)-4-fluorophenyl)-1-methylurea as an off white solid. LCMS m/z found 468.1 [M+H]⁺; RT=5.63 min, (Method A); ¹H (400 MHz, DMSO-d6): δ 8.66 (s, 1H), 8.17 (m, 1H), 7.88-7.83 (m, 1H), 7.75-7.70 (m, 2H), 7.35-7.05 (m, 2H), 5.69 (s, 1H), 4.82 (d, 1H), 4.67 (d, 1H), 4.17 (d, 1H), 4.06 (s, 3H), 3.99 (m, 1H), 2.77 (s, 3H). Chiral analytical SFC: RT=1.22 min, Column: ChiralPak IC-3 (4.6×150 mm) 3 μm, 30% (0.5% DEA in methanol), Flow rate: 3 g/min.

### (S)-3-(3-Chloro-4-fluorophenyl)-1-(8,9-difluoro-6-methoxy-1,4-dihydro-2H-pyrano[3,4-c]isoquinolin-1-yl)-1-methylurea (Compound 244)

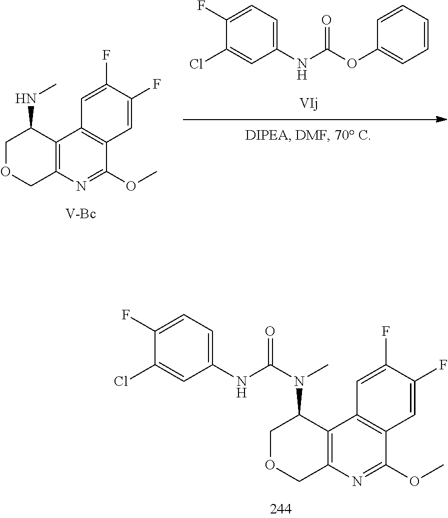 [SMILES: CN[C@@H]1COCc2nc(OC)c3cc(F)c(F)cc3c21 ; O=C(Nc1ccc(F)c(Cl)c1)Oc1ccccc1 ; COc1nc2c(c3cc(F)c(F)cc13)[C@H](N(C)C(=O)Nc1ccc(F)c(Cl)c1)COC2]

(S)-3-(3-Chloro-4-fluorophenyl)-1-(8,9-difluoro-6-methoxy-1,4-dihydro-2H-pyrano[3,4-c]isoquinolin-1-yl)-1-methylurea was synthesized in an analogous manner as above from crude (S)-8,9-difluoro-6-methoxy-N-methyl-1,4-dihydro-2H-pyrano[3,4-c]isoquinolin-1-amine (V-Bc) and phenyl (3-chloro-4-fluorophenyl)carbamate (VIj). LCMS m/z found 452.1/454.0 [M+H]⁺; RT=5.89 min, (Method A); ¹H (400 MHz, DMSO-d6): δ 8.61 (s, 1H), 8.17 (m, 1H), 7.86 (m, 1H), 7.72 (m, 1H), 7.35-7.55 (m, 1H), 7.37 (m, 1H), 5.68 (s, 1H), 4.82 (d, 1H), 4.67 (d, 1H), 4.16 (d, 1H), 4.02 (s, 3H), 3.99 (m, 1H), 2.76 (s, 3H). Chiral analytical SFC: RT=2.057 min, Column: ChiralPakl AD-3 (4.6×150 mm) 3 μm, 30% (0.5% DEA in methanol), Flow rate: 3 g/min.

### 2-(((S)-8,9-Difluoro-1-(((R)-1-(4-methoxyphenyl)ethyl)(methyl)amino)-1,4-dihydro-2H-pyrano[3,4-c]isoquinolin-6-yl)oxy)ethyl acetate (XIg) and 2-((S)-8,9-Difluoro-1-(((R)-1-(4-methoxyphenyl)ethyl)(methyl)amino)-6-oxo-1,2,4,6-tetrahydro-5H-pyrano[3,4-c]isoquinolin-5-yl)ethyl acetate (XIh)

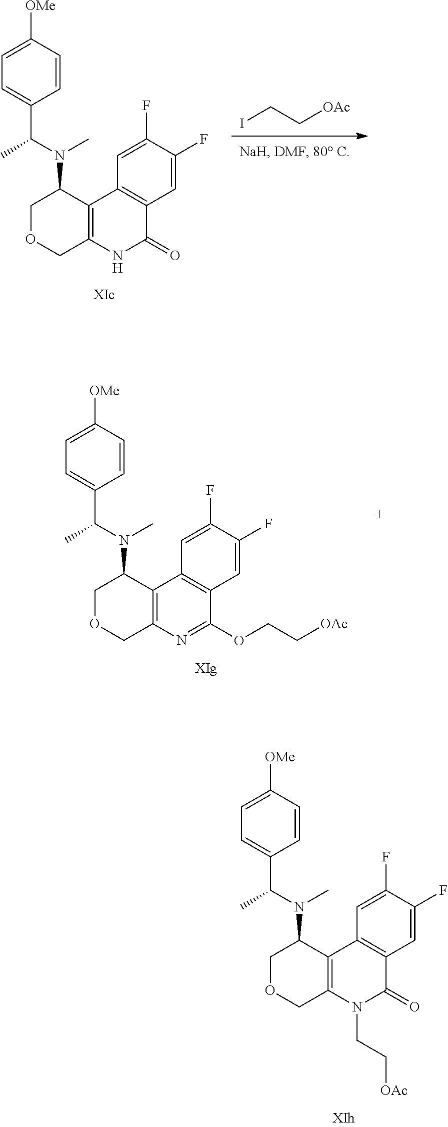

To a stirred solution of 2.0 g (5.0 mmol, 1.0 eq.) of 8,9-difluoro-1-(((R)-1-(4-methoxyphenyl)ethyl) (methyl)amino)-1,5-dihydro-2H-pyrano[3,4-c]isoquinolin-6(4H)-one (XIc) in 20 mL of DMF was added 180 mg of NaH (7.5 mmol, 1.5 eq.) at 0° C. and the reaction mixture was stirred for 15 min. 2.44 g of 2-iodoethyl acetate (10.0 mmol, 2.0 eq.) were added and the reaction mixture was heated at 80° C. for 3 h. After completion of the reaction, the mixture was cooled to room temperature, quenched with ice-cold water, and extracted with ethyl acetate (2×200 mL). Combined organic layers were washed with ice-cold water (100 mL), dried over anhydrous sodium sulfate, filtered, and the volatiles were evaporated. Column chromatography (Silica gel 100-200 mesh, 20% ethyl acetate in petroleum ether as a linear gradient) afforded 410 mg (17% yield) of 2-(8,9-difluoro-1-(((R)-1-(4-methoxyphenyl)ethyl)(methyl)amino)-6-oxo-1,2,4,6-tetrahydro-5H-pyrano[3,4-c]isoquinolin-5-yl)ethyl acetate (XIg) and 430 mg (18% yield) of 2-(8,9-difluoro-1-(((R)-1-(4-methoxyphenyl)ethyl)(methyl)amino)-6-oxo-1,2,4,6-tetrahydro-5H-pyrano[3,4-c]isoquinolin-5-yl)ethyl acetate (XIh), as off white solids. XIg: LCMS: m/z found 487.32 [M+H]⁺, RT=1.71 min, (Method D); XIh: LCMS: m/z found 487.32 [M+H]⁺, RT=1.55 min, (Method D).

### Benzyl (2-(8,9-difluoro-1-(((R)-1-(4-methoxyphenyl)ethyl)(methyl)amino)-6-oxo-1,2,4,6-tetrahydro-5H-pyrano[3,4-c]isoquinolin-5-yl)ethyl)carbamate (XIi) and Benzyl (2-((8,9-difluoro-1-(((R)-1-(4-methoxyphenyl)ethyl)(methyl)amino)-1,4-dihydro-2H-pyrano[3,4-c]isoquinolin-6-yl)oxy)ethyl)carbamate (XIj)

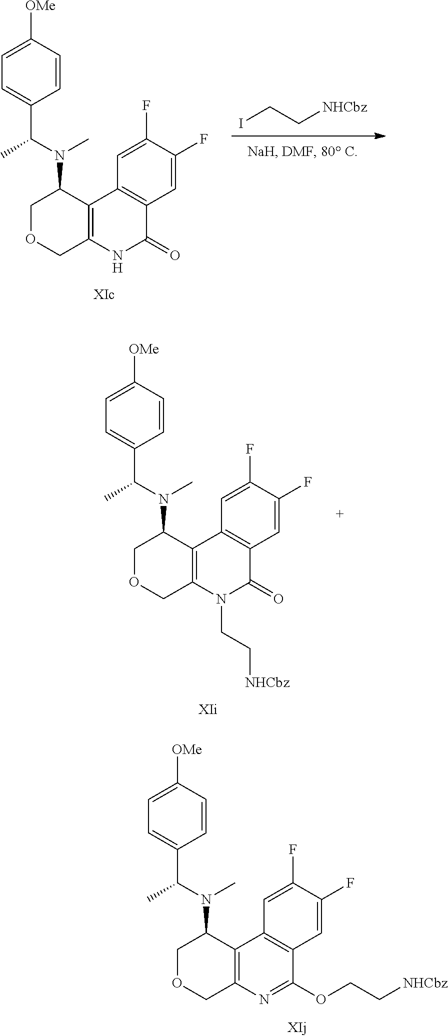

8,9-Difluoro-1-(((R)-1-(4-methoxyphenyl)ethyl) (methyl)amino)-1,5-dihydro-2H-pyrano[3,4-c]isoquinolin-6(4H)-one (XIc) was converted to benzyl (2-(8,9-difluoro-1-(((R)-1-(4-methoxyphenyl)ethyl)(methyl)amino)-6-oxo-1,2,4,6-tetrahydro-5H-pyrano[3,4-c]isoquinolin-5-yl)ethyl)carbamate (XIi) and benzyl (2-((8,9-difluoro-1-(((R)-1-(4-methoxyphenyl)ethyl)(methyl)amino)-1,4-dihydro-2H-pyrano[3,4-c]isoquinolin-6-yl)oxy)ethyl)carbamate (XIj) in an analogous manner as above. XIi: LCMS: m/z found 578.70 [M+H]⁺, RT=1.62 min, (Method D); XIj: LCMS: m/z found 578.70 [M+H]⁺, RT=1.77 min, (Method D).

### 2-((8,9-Difluoro-1-(methylamino)-1,4-dihydro-2H-pyrano[3,4-c]isoquinolin-6-yl)oxy)ethyl acetate (V-Be)

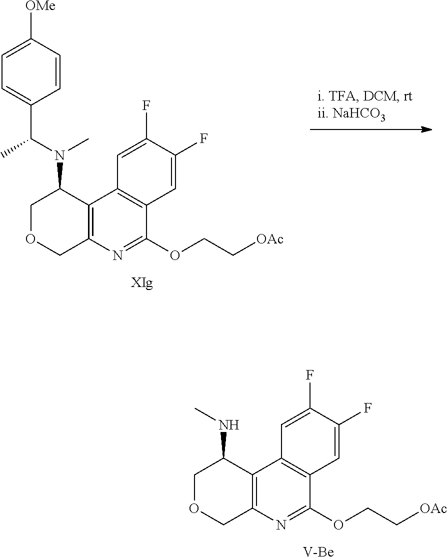

To a stirred solution of 350 mg (0.72 mmol, 1.0 eq.) of 2-(8,9-difluoro-1-(((R)-1-(4-methoxyphenyl)ethyl)(methyl)amino)-6-oxo-1,2,4,6-tetrahydro-5H-pyrano[3,4-c]isoquinolin-5-yl)ethyl acetate (XIg) in 3.5 mL of methylene chloride was added 1.72 mL of trifluoroacetic acid at 0° C. and the mixture was stirred for 12 h. After completion of the reaction, the mixture was quenched with saturated sodium bicarbonate and extracted with ethyl acetate (2×50 mL). Combined organic layers were washed with ice-cold water, dried over anhydrous Na₂SO₄ and evaporated to obtained 280 mg 2-((8,9-difluoro-1-(methylamino)-1,4-dihydro-2H-pyrano[3,4-c]isoquinolin-6-yl)oxy)ethyl acetate. LCMS: m/z found 352.9 [M+H]⁺, RT=1.26 min, (Method D).

### Benzyl (S)-(2-((8,9-difluoro-1-(methylamino)-1,4-dihydro-2H-pyrano[3,4-c]isoquinolin-6-yl)oxy)ethyl)carbamate (V-Bf)

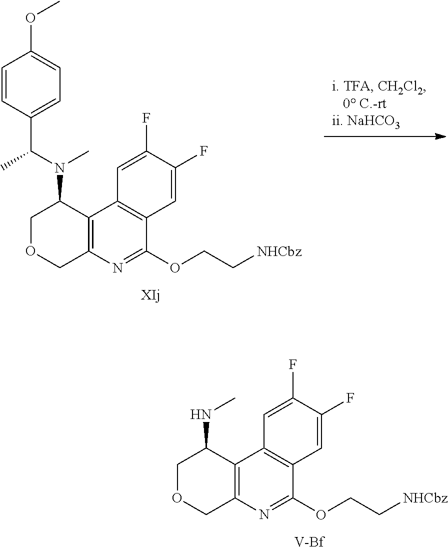

Benzyl (S)-(2-((8,9-difluoro-1-(methylamino)-1,4-dihydro-2H-pyrano[3,4-c]isoquinolin-6-yl)oxy)ethyl)carbamate was synthesized in an analogous manner as above from benzyl (2-((8,9-difluoro-1-(((R)-1-(4-methoxyphenyl)ethyl)(methyl)amino)-1,4-dihydro-2H-pyrano[3,4-c]isoquinolin-6-yl)oxy)ethyl)carbamate (XIj). LCMS: m/z found 444.32 [M+H]⁺, RT=2.02 min, (Method D).

### 2-(8,9-Difluoro-1-(methylamino)-6-oxo-1,2,4,6-tetrahydro-5H-pyrano[3,4-c]isoquinolin-5-yl)ethyl acetate (Vaab)

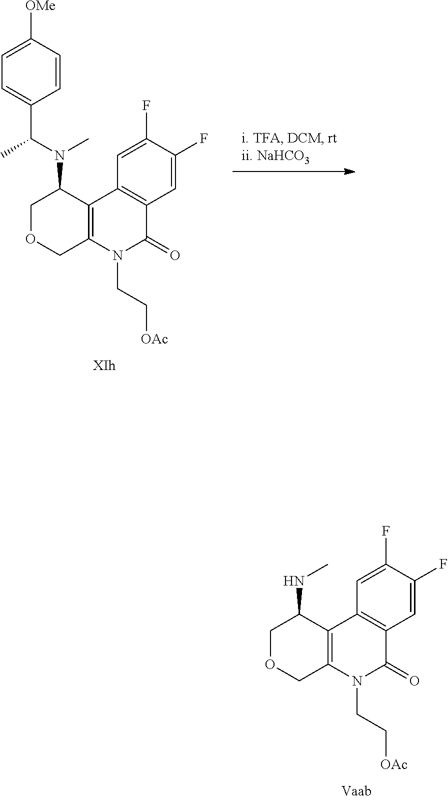 [SMILES: COc1ccc([C@@H](C)N(C)[C@@H]2COCc3c2c2cc(F)c(F)cc2c(=O)n3CCOC(C)=O)cc1 ; CN[C@@H]1COCc2c1c1cc(F)c(F)cc1c(=O)n2CCOC(C)=O]

2-(8,9-Difluoro-1-(methylamino)-6-oxo-1,2,4,6-tetrahydro-5H-pyrano[3,4-c]isoquinolin-5-yl)ethyl acetate (Vaab) was synthesized in an analogous manner as described above, from 2-(8,9-difluoro-1-(((R)-1-(4-methoxyphenyl)ethyl)(methyl)amino)-6-oxo-1,2,4,6-tetrahydro-5H-pyrano[3,4-c]isoquinolin-5-yl)ethyl acetate (XIh). LCMS m/z found 352.9 [M+H]⁺; RT=1.01 min, (Method D).

### Benzyl (2-(8,9-difluoro-1-(methylamino)-6-oxo-1,2,4,6-tetrahydro-5H-pyrano[3,4-c]isoquinolin-5-yl)ethyl)carbamate (Vaac)

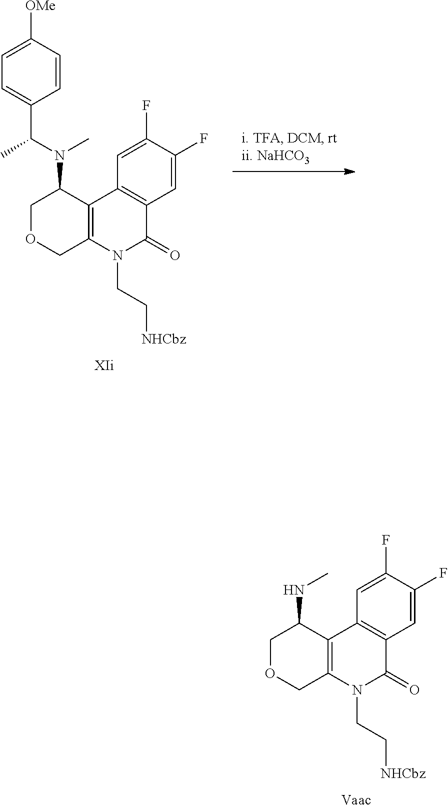 [SMILES: COc1ccc([C@@H](C)N(C)[C@@H]2COCc3c2c2cc(F)c(F)cc2c(=O)n3CCNC(=O)OCc2ccccc2)cc1 ; CN[C@@H]1COCc2c1c1cc(F)c(F)cc1c(=O)n2CCNC(=O)OCc1ccccc1]

Benzyl (2-(8,9-difluoro-1-(methylamino)-6-oxo-1,2,4,6-tetrahydro-5H-pyrano[3,4-c]isoquinolin-5-yl)ethyl)carbamate (Vaac) was synthesized in an analogous manner as described above, from benzyl (2-(8,9-difluoro-1-(((R)-1-(4-methoxyphenyl)ethyl)(methyl)amino)-6-oxo-1,2,4,6-tetrahydro-5H-pyrano[3,4-c]isoquinolin-5-yl)ethyl)carbamate (XIi). LCMS m/z found 444.32 [M+H]⁺; RT=1.35 min, (Method D).

### (S)-1-(8,9-Difluoro-6-(2-hydroxyethoxy)-1,4-dihydro-2H-pyrano[3,4-c]isoquinolin-1-yl)-3-(3-(difluoromethyl)-4-fluorophenyl)-1-methylurea (Compound 256)

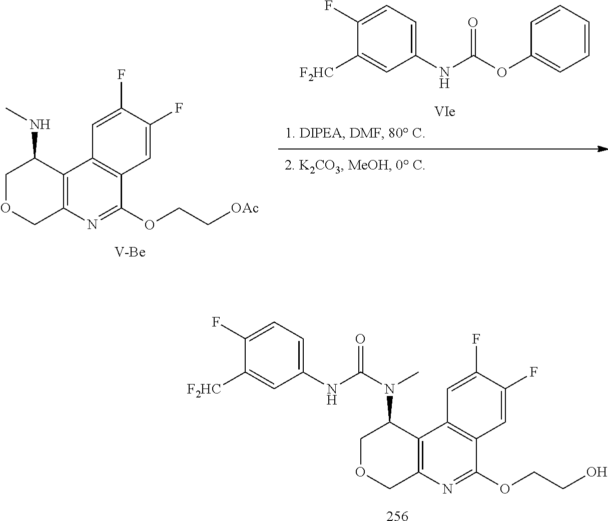

Step 1. To a stirred solution of 50 mg (0.14 mmol, 1.0 eq.) of 2-(8,9-difluoro-1-(methylamino)-6-oxo-1,2,4,6-tetrahydro-5H-pyrano[3,4-c]isoquinolin-5-yl)ethyl acetate (V-Be) and 39 mg (0.14 mmol, 1.0 eq.) of phenyl (3-(difluoromethyl)-4-fluorophenyl)carbamate (VIe) in 1 mL of DMF were added 0.07 mL (0.42 mmol, 2.0 eq.) of DIPEA at room temperature. The resulting mixture was stirred at 80° C. for 1 h. After completion of reaction, the mixture was cooled and poured into ice-cold water (20 mL) and stirred for 30 min. The resulting solid was collected by filtration, washed with Et₂O (2×10 mL) and dried under vacuum to afford 55 mg of crude (S)-2-((1-(3-(3-(difluoromethyl)-4-fluorophenyl)-1-methylureido)-8,9-difluoro-1,4-dihydro-2H-pyrano[3,4-c]isoquinolin-6-yl)oxy)ethyl acetate, which was used in the next step without further purification. LCMS: m/z found 539.1 [M−1]⁻, RT=2.21 min, (Method D). Step 2. To a stirred solution of 50 mg (0.092 mmol, 1.0 eq.) (S)-2-((1-(3-(3-(difluoromethyl)-4-fluorophenyl)-1-methylureido)-8,9-difluoro-1,4-dihydro-2H-pyrano[3,4-c]isoquinolin-6-yl)oxy)ethyl acetate in 1 mL of MeOH was added 24 mg of K₂CO₃ (0.18 mmol, 2.0 eq.) at 0° C. and the reaction mixture was stirred at room temperature for 2 h. After completion of the reaction, the mixture was diluted with 10 ml of MeOH and filtered and the filtrate was evaporated. The product was purified by column chromatography (Silica gel (60-120 mesh, 60% ethyl acetate in petroleum ether as a linear gradient) to afford 26 mg of (S)-1-(8,9-difluoro-6-(2-hydroxyethoxy)-1,4-dihydro-2H-pyrano[3,4-c]isoquinolin-1-yl)-3-(3-(difluoromethyl)-4-fluorophenyl)-1-methylurea (single enantiomer) as an off white solid. LCMS m/z found 498.17 [M+H]⁺; RT=1.94 min, (Method D); ¹H (400 MHz, DMSO-d6): δ 8.64 (s, 1H), 8.32 (t, 1H), 7.89 (d, 1H), 7.74 (m, 2H), 7.35-7.05 (m, 2H), 5.69 (s, 1H), 5.01 (t, 1H), 4.79-4.61 (m, 2H), 4.46 (t, 2H), 4.17-4.02 (m, 2H), 3.84 (t, 2H), 2.76 (s, 3H). Chiral analytical SFC: RT=2.15 min, Column: Chiralcel IC-3 (4.6×150 mm) 3 μm, 30% (0.5% DEA in methanol), Flow rate: 3 g/min.

### (S)-3-(3-Chloro-4-fluorophenyl)-1-(8,9-difluoro-6-(2-hydroxyethoxy)-1,4-dihydro-2H-pyrano[3,4-c]isoquinolin-1-yl)-1-methylurea (Compound 255)

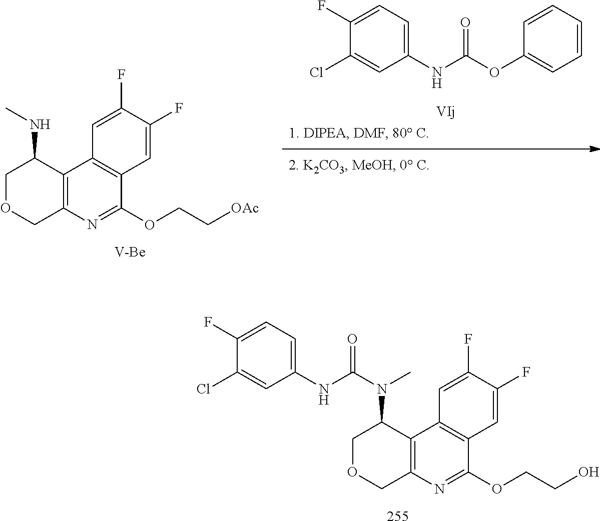

(S)-3-(3-Chloro-4-fluorophenyl)-1-(8,9-difluoro-6-(2-hydroxyethoxy)-1,4-dihydro-2H-pyrano[3,4-c]isoquinolin-1-yl)-1-methylurea was synthesized in an analogous manner as above from 2-(8,9-difluoro-1-(methylamino)-6-oxo-1,2,4,6-tetrahydro-5H-pyrano[3,4-c]isoquinolin-5-yl)ethyl acetate (V-Be) and (3-chloro-4-fluorophenyl)carbamate (VIj). LCMS m/z found 482.10 [M+H]⁺; RT=2.02 min, (Method D); ¹H (400 MHz, DMSO-d6): δ 8.61 (s, 1H), 8.32 (t, 1H), 7.86 (d, 1H), 7.73 (1, 2H), 7.55 (1, 2H), 7.37 (t, 1H), 5.68 (s, 1H), 5.01 (t, 1H), 4.79-4.61 (m, 2H), 4.46 (t, 2H), 4.16-4.01 (m, 2H), 3.84 (t, 2H), 2.75 (s, 3H). Chiral analytical SFC: RT=4.41 min, Column: Chiralcel IC-3 (4.6×150 mm) 3 μm, 30% (0.5% DEA in methanol), Flow rate: 3 g/min.

### (S)-1-(8,9-Difluoro-5-(2-hydroxyethyl)-6-oxo-1,4,5,6-tetrahydro-2H-pyrano[3,4-c]isoquinolin-1-yl)-3-(3-(difluoromethyl)-4-fluorophenyl)-1-methylurea (Compound 251)

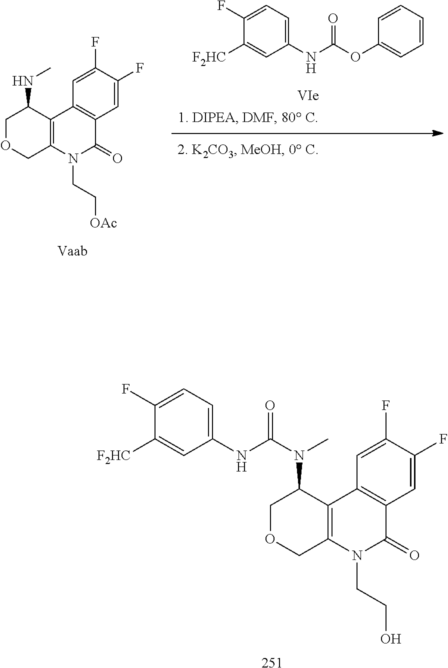 [SMILES: CN[C@@H]1COCc2c1c1cc(F)c(F)cc1c(=O)n2CCOC(C)=O ; O=C(Nc1ccc(F)c(C(F)F)c1)Oc1ccccc1 ; CN(C(=O)Nc1ccc(F)c(C(F)F)c1)[C@@H]1COCc2c1c1cc(F)c(F)cc1c(=O)n2CCO ; "251"]

(S)-1-(8,9-Difluoro-5-(2-hydroxyethyl)-6-oxo-1,4,5,6-tetrahydro-2H-pyrano[3,4-c]isoquinolin-1-yl)-3-(3-(difluoromethyl)-4-fluorophenyl)-1-methylurea was synthesized in an analogous manner as above from 2-(8,9-difluoro-1-(methylamino)-6-oxo-1,2,4,6-tetrahydro-5H-pyrano[3,4-c]isoquinolin-5-yl)ethyl acetate (Vaab) and phenyl (3-(difluoromethyl)-4-fluorophenyl)carbamate (VIe). LCMS m/z found 496.35 [M−H]⁻; RT=1.82 min, (Method D); ¹H (400 MHz, DMSO-d6): δ 8.64 (s, 1H), 8.19 (t, 1H), 7.88 (d, 1H), 7.74 (d, 1H), 7.52 (m, 1H), 7.34-7.07 (m, 2H), 5.46 (s, 1H), 5.14-5.02 (m, 2H), 4.70 (d, 1H), 4.07-3.84 (m, 6H), 2.82 (s, 3H). Chiral analytical SFC: RT=1.36 min, Column: Chiralcel OX-3 (4.6×150 mm) 3 μm, 30% (0.5% DEA in methanol), Flow rate: 3 g/min.

### (S)-3-(3-Chloro-4-fluorophenyl)-1-(8,9-difluoro-5-(2-hydroxyethyl)-6-oxo-1,4,5,6-tetrahydro-2H-pyrano[3,4-c]isoquinolin-1-yl)-1-methylurea (Compound 254)

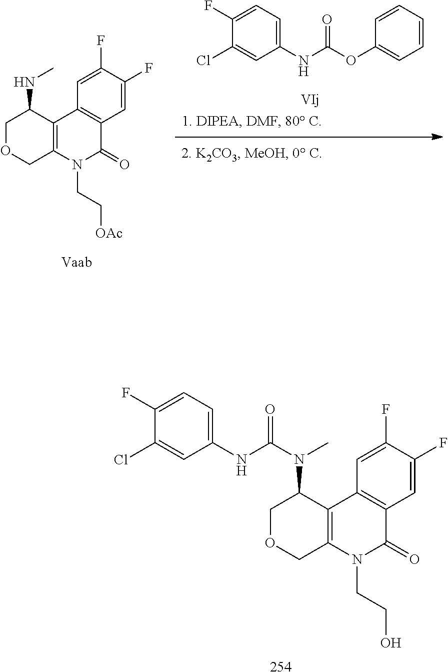 [SMILES: CN[C@@H]1COCc2c1c1cc(F)c(F)cc1c(=O)n2CCOC(C)=O ; O=C(Nc1ccc(F)c(Cl)c1)Oc1ccccc1 ; CN(C(=O)Nc1ccc(F)c(Cl)c1)[C@@H]1COCc2c1c1cc(F)c(F)cc1c(=O)n2CCO ; "254"]

(S)-3-(3-Chloro-4-fluorophenyl)-1-(8,9-difluoro-5-(2-hydroxyethyl)-6-oxo-1,4,5,6-tetrahydro-2H-pyrano[3,4-c]isoquinolin-1-yl)-1-methylurea was synthesized in an analogous manner as above from 2-(8,9-difluoro-1-(methylamino)-6-oxo-1,2,4,6-tetrahydro-5H-pyrano[3,4-c]isoquinolin-5-yl)ethyl acetate (Vaab) and (3-chloro-4-fluorophenyl)carbamate (VIj). LCMS m/z found 482.41 [M−H]; RT=1.87 min, (Method D); ¹H (400 MHz, DMSO-d6): δ 8.59 (s, 1H), 8.19 (t, 1H), 7.86 (m, 1H), 7.54-7.32 (m, 3H), 5.45 (s, 1H), 5.14-5.02 (m, 2H), 4.69 (d, 1H), 4.06-3.84 (m, 6H), 2.81 (s, 3H). Chiral analytical SFC: RT=1.92 min, Column: Chiralcel OX-3 (4.6×150 mm) 3 μm, 30% (0.5% DEA in methanol), Flow rate: 3 g/min.

### (S)-1-(6-(2-Aminoethoxy)-8,9-difluoro-1,4-dihydro-2H-pyrano[3,4-c]isoquinolin-1-yl)-3-(3-(difluoromethyl)-4-fluorophenyl)-1-methylurea (Compound 252)

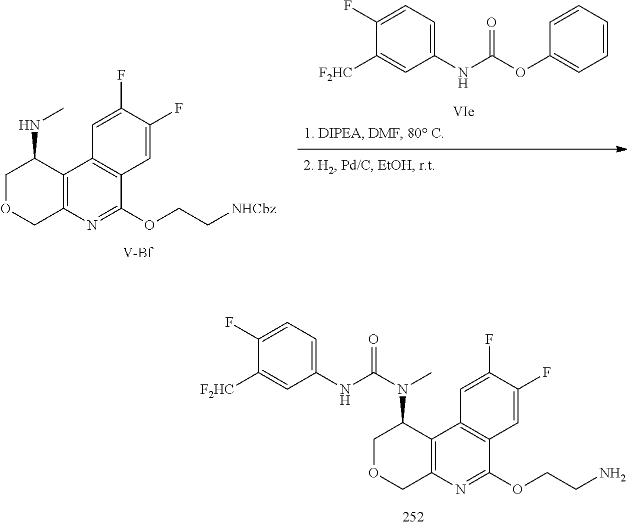

Step 1. Benzyl (S)-(2-((1-(3-(3-(difluoromethyl)-4-fluorophenyl)-1-methylureido)-8,9-difluoro-1,4-dihydro-2H-pyrano[3,4-c]isoquinolin-6-yl)oxy)ethyl)carbamate was synthesized in an analogous manner as above from benzyl (S)-(2-((8,9-difluoro-1-(methylamino)-1,4-dihydro-2H-pyrano[3,4-c]isoquinolin-6-yl)oxy)ethyl)carbamate (V-Bf) and phenyl (3-(difluoromethyl)-4-fluorophenyl)carbamate (VIe). LCMS m/z found 631.2 [M−H]; RT=2.10 min, (Method D). Step 2. To a stirred solution of 150 mg (0.23 mmol, 1.0 eq.) of benzyl (S)-(2-((1-(3-(3-(difluoromethyl)-4-fluorophenyl)-1-methylureido)-8,9-difluoro-1,4-dihydro-2H-pyrano[3,4-c]isoquinolin-6-yl)oxy)ethyl)carbamate (from Step 1) in 10 mL of EtOH was added 80 mg (0.11 mmol, 0.6 eq.) of Pd/C and the mixture was stirred for 6 h under an atmosphere of hydrogen (balloon). After completion of the reaction, the mixture was filtered through a pad of celite and washed with 10% DMF/MeOH (20 mL). Combined filtrate was concentrated under reduced pressure and crude material was purified by preparative HPLC to afford 12 mg of (S)-1-(6-(2-aminoethoxy)-8,9-difluoro-1,4-dihydro-2H-pyrano[3,4-c]isoquinolin-1-yl)-3-(3-(difluoromethyl)-4-fluorophenyl)-1-methylurea (single enantiomer) as an off white solid. LCMS m/z found 495.42 [M−H]; RT=5.26 min, (Method: Mobile phase-A: 10 mM Ammonium Acetate in Water Mobile phase-B: ACN., Column—Ascentis R Express C18, 2.7 μm, 50×2.1 mm, Flow-0.5 mL/min, Temp: 40° C., Time (min) and % B:0-3;0.3-3;2.5-97;3.7-97;4-3;4.6-3); ¹H (400 MHz, DMSO-d6): δ 8.67 (s, 1H), 8.35 (t, 1H), 7.88 (d, 1H), 7.73-7.07 (m, 4H), 5.68 (s, 1H), 4.79 (d, 1H), 4.65 (d, 1H), 4.37 (bs, 2H), 4.16-4.02 (m, 2H), 2.97 (bs, 2H), 2.76 (s, 3H). Chiral analytical SFC: RT=3.68 min, Column: Chiralcel IC-3 (4.6×150 mm) 3 μm, 25% (0.5% DEA in methanol), Flow rate: 3 g/min.

### (S)-1-(5-(2-Aminoethyl)-8,9-difluoro-6-oxo-1,4,5,6-tetrahydro-2H-pyrano[3,4-c]isoquinolin-1-yl)-3-(3-(difluoromethyl)-4-fluorophenyl)-1-methylurea (Compound 253)

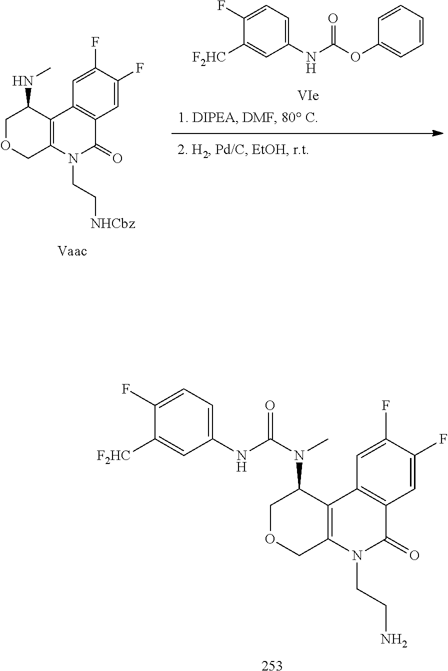 [SMILES: CN[C@@H]1COCc2c1c1cc(F)c(F)cc1c(=O)n2CCNC(=O)OCc1ccccc1 ; O=C(Nc1ccc(F)c(C(F)F)c1)Oc1ccccc1 ; CN(C(=O)Nc1ccc(F)c(C(F)F)c1)[C@@H]1COCc2c1c1cc(F)c(F)cc1c(=O)n2CCN ; "253"]

(S)-1-(5-(2-Aminoethyl)-8,9-difluoro-6-oxo-1,4,5,6-tetrahydro-2H-pyrano[3,4-c]isoquinolin-1-yl)-3-(3-(difluoromethyl)-4-fluorophenyl)-1-methylurea was synthesized in an analogous manner as above from benzyl (2-(8,9-difluoro-1-(methylamino)-6-oxo-1,2,4,6-tetrahydro-5H-pyrano[3,4-c]isoquinolin-5-yl)ethyl)carbamate (Vaac) and phenyl (3-(difluoromethyl)-4-fluorophenyl)carbamate (VIe). LCMS m/z found 497.45 [M+H]⁺, RT=1.52 min, (Method: Mobile phase-A: 10 mM Ammonium Acetate in Water Mobile phase-B: ACN., Column—Ascentis R Express C18, 2.7 μm, 50×2.1 mm, Flow-0.5 mL/min, Temp: 40° C., Time (min) and % B: 0-3;0.3-3;2.5-97;3.7-97;4-3;4.6-3); ¹H (400 MHz, DMSO-d6): δ 8.65 (s, 1H), 8.18 (t, 1H), 7.88 (d, 1H), 7.73 (d, 1H), 7.51 (m, 1H), 7.34-7.07 (m, 2H), 5.45 (s, 1H), 5.12 (d, 1H), 4.71 (d, 1H), 4.07-3.74 (m, 4H), 2.82 (s, 5H), 1.23 (bs, 2H). Chiral analytical SFC: RT=2.98 min, Column: Chiralcel OX-3 (4.6×150 mm) 3 μm, 30% (0.5% DEA in methanol), Flow rate: 3 g/min.

### (S)-1-(5-(2-Aminoethyl)-8,9-difluoro-6-oxo-1,4,5,6-tetrahydro-2H-pyrano[3,4-c]isoquinolin-1-yl)-3-(3-chloro-4-fluorophenyl)-1-methylurea (Compound 257)

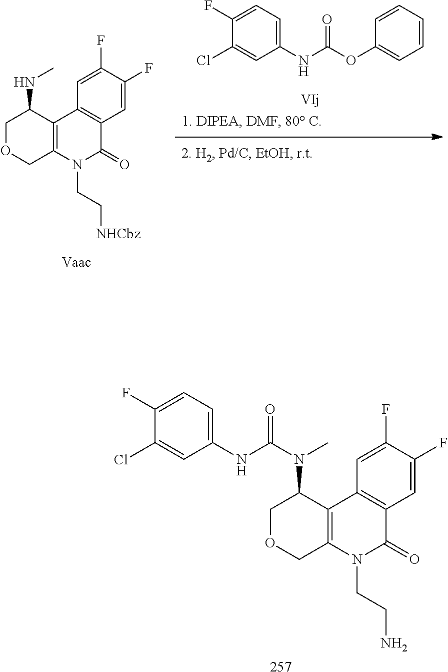 [SMILES: CN[C@@H]1COCc2c1c1cc(F)c(F)cc1c(=O)n2CCNC(=O)OCc1ccccc1 ; O=C(Nc1ccc(F)c(Cl)c1)Oc1ccccc1 ; CN(C(=O)Nc1ccc(F)c(Cl)c1)[C@@H]1COCc2c1c1cc(F)c(F)cc1c(=O)n2CCN ; "257"]

(S)-1-(5-(2-Aminoethyl)-8,9-difluoro-6-oxo-1,4,5,6-tetrahydro-2H-pyrano[3,4-c]isoquinolin-1-yl)-3-(3-chloro-4-fluorophenyl)-1-methylurea was synthesized in an analogous manner as above from benzyl (2-(8,9-difluoro-1-(methylamino)-6-oxo-1,2,4,6-tetrahydro-5H-pyrano[3,4-c]isoquinolin-5-yl)ethyl)carbamate (Vaac) and (3-chloro-4-fluorophenyl)carbamate (VIj). LCMS m/z found 481.11 [M+H]⁺, RT=1.54 min, (Method: Mobile phase-A: 10 mM Ammonium Acetate in Water Mobile phase-B: ACN., Column—Ascentis R Express C18, 2.7 μm, 50×2.1 mm, Flow-0.5 mL/min, Temp: 40° C., Time (min) and % B: 0-3;0.3-3;2.5-97;3.7-97;4-3;4.6-3); ¹H (400 MHz, DMSO-d6): δ 8.59 (s, 1H), 8.18 (t, 1H), 7.86-7.83 (m, 1H), 7.54-7.32 (m, 3H), 5.44 (s, 1H), 5.11 (d, 1H), 4.70 (d, 1H), 4.07-3.82 (m, 4H), 2.82 (s, 5H), 1.23 (bs, 2H). Chiral analytical SFC: RT=3.59 min, Column: Chiralcel OX-3 (4.6×150 mm) 3 μm, 30% (0.5% DEA in methanol), Flow rate: 3 g/min.

### (S)-1-(6-(2-Aminoethoxy)-8,9-difluoro-1,4-dihydro-2H-pyrano[3,4-c]isoquinolin-1-yl)-3-(3-chloro-4-fluorophenyl)-1-methylurea (Compound 258)

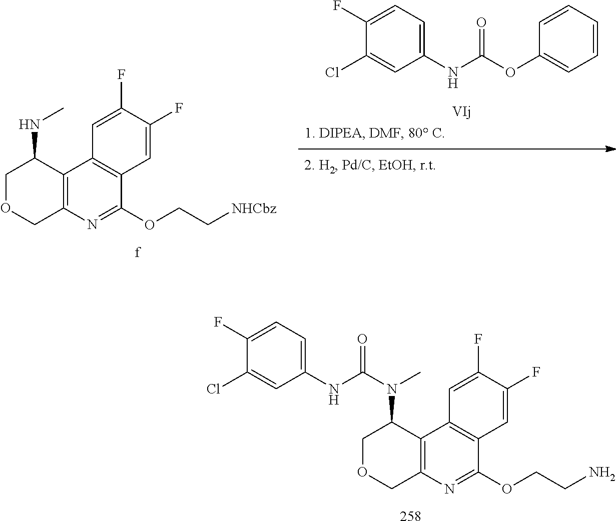

(S)-1-(6-(2-Aminoethoxy)-8,9-difluoro-1,4-dihydro-2H-pyrano[3,4-c]isoquinolin-1-yl)-3-(3-chloro-4-fluorophenyl)-1-methylurea was synthesized in an analogous manner as above from benzyl (S)-(2-((8,9-difluoro-1-(methylamino)-1,4-dihydro-2H-pyrano[3,4-c]isoquinolin-6-yl)oxy)ethyl)carbamate (V-Bf) and (3-chloro-4-fluorophenyl)carbamate (VIj). LCMS m/z found 481.41 [M+H]⁺, RT=1.51 min, (Method: Mobile phase-A: 10 mM Ammonium Acetate in Water Mobile phase-B: ACN., Column—Ascentis R Express C18, 2.7 μm, 50×2.1 mm, Flow-0.5 mL/min, Temp: 40° C., Time (min) and % B: 0-3;0.3-3;2.5-97;3.7-97;4-3;4.6-3); ¹H (400 MHz, DMSO-d6): δ 8.62 (s, 1H), 8.34 (t, 1H), 7.86 (m, 1H), 7.72 (m, 1H), 7.55 (m, 1H) 7.37 (t, 1H), 5.68 (s, 1H), 4.79 (d, 1H), 4.65 (d, 1H), 4.37 (bs, 2H), 4.16-4.02 (m, 2H), 2.97 (bs, 2H), 2.75 (s, 3H), 1.98 (bs, 2H). Chiral analytical SFC: RT=4.25 min, Column: Chiralcel IC-3 (4.6×150 mm) 3 μm, 25% (0.5% DEA in methanol), Flow rate: 3 g/min.

### (S)-3-(3-Chloro-4-fluorophenyl)-1-(8,9-difluoro-6-oxo-1,4,5,6-tetrahydro-2H-pyrano[3,4-c]isoquinolin-1-yl-1-d)-1-(methyl-d3)urea: Enantiomer II (Compound 146)

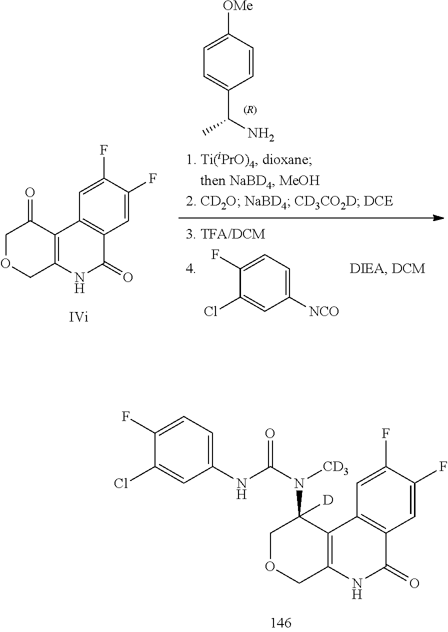

C₃ (S)-3-(3-Chloro-4-fluorophenyl)-1-(8,9-difluoro-6-oxo-1,4,5,6-tetrahydro-2H-pyrano[3,4-c]isoquinolin-1-yl-1-d)-1-(methyl-d3)urea: Enantiomer II was synthesized by an analogous chiral synthesis sequence as described above for Compound 72, starting from 8,9-difluoro-4,5-dihydropyrano[3,4-c]isoquinoline-1,6-dione (IVi), except for using deuterium-labeled reagents: NaBD₄, CD₂O, and CD₃CO₂D. LCMS m/z found 442.2/444.2 [M+H]⁺; RT=4.25 min (Method A); ¹H NMR (400 MHz, DMSO-d₆) δ 11.66 (s, 1H), 8.59 (s, 1H), 8.10 (dd, 1H), 7.84 (dd, 1H), 7.59-7.41 (m, 2H), 7.34 (t, 1H), 4.59 (d, 1H), 4.42 (d, 1H), 4.10-4.04 (m, 1H), 3.93 (d, 1H).

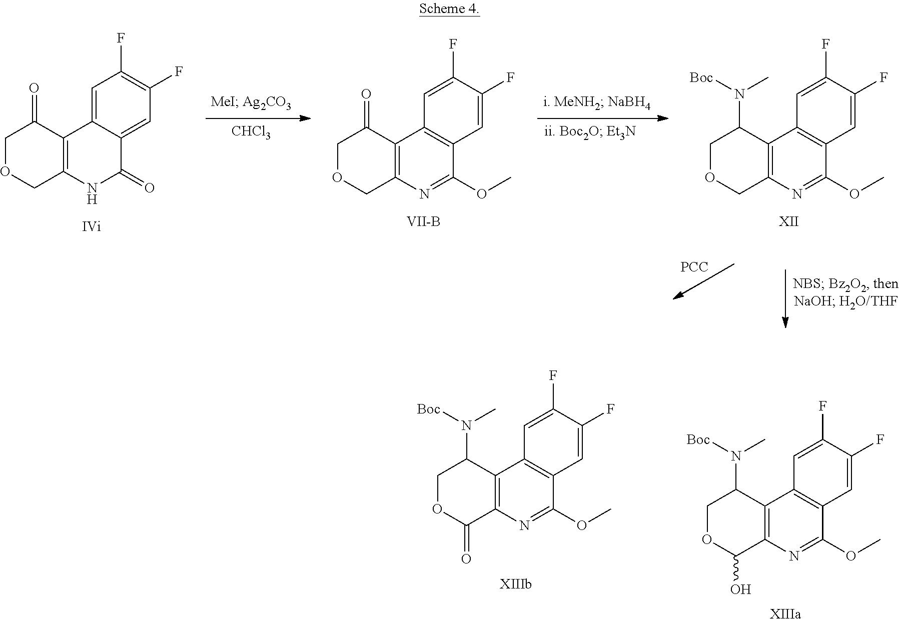

### 8,9-Difluoro-6-methoxy-2H-pyrano[3,4-c]isoquinolin-1(4H)-one (VII-B)

8,9-Difluoro-4,5-dihydropyrano[3,4-c]isoquinoline-1,6-dione (IVi, 1 g, 4.0 mmol), iodomethane, (3.25 mL, 52.2 mmol), and silver carbonate (2.74 g, 10 mmol) were stirred in chloroform (150 mL) at 55° C. under an atmosphere of nitrogen for 48 h. The reaction mixture was cooled to room temperature, diluted with dichloromethane, and filtered through CELITE®. The solvent was evaporated under reduced pressure and the regioisomeric products were separated by flash chromatography (Silicagel, EtOAc/Hexanes 0-30%), to afford 8,9-difluoro-6-methoxy-2H-pyrano[3,4-c]isoquinolin-1(4H)-one (VII-B in Scheme 4; 497 mg, 47% yield) as the major regioisomer: ¹H NMR (400 MHz, Chloroform-d) δ 9.16 (dd, 1H), 8.00 (dd, 1H), 4.94 (d, 2H), 4.39 (dd, 2H), 4.16 (s, 3H), and 8,9-difluoro-5-methyl-4H-pyrano[3,4-c]isoquinoline-1,6-dione as the minor isomer: ¹H NMR (400 MHz, Chloroform-d) δ 9.05 (dd, 1H), 8.18 (dd, 1H), 4.93 (s, 2H), 4.45-4.22 (m, 2H), 3.58 (s, 3H).

### tert-Butyl N-(8,9-Difluoro-6-methoxy-2,4-dihydro-1H-pyrano[3,4-c]isoquinolin-1-yl)-N-methyl-carbamate (XII)

Step i. Tetraisopropoxytitanium (915 μL, 3.0 mmol) was added to a mixture of 8,9-difluoro-6-methoxy-4H-pyrano[3,4-c]isoquinolin-1-one (VII-B, 200 mg, 0.75 mmol) and a 2 M solution of methylamine (1.13 mL, 2.26 mmol) in THF, combined in 1,4-dioxane (10 mL). The mixture was stirred under nitrogen for 2 h at 65° C. The reaction mixture was then diluted with 4 mL anhydrous methanol, and allowed to cool in an ice bath. Sodium borohydride (57 mg, 1.5 mmol) was added in one portion. The reaction mixture was stirred for 5 minutes, and the ice bath was removed. After an additional 1 h, the reaction was quenched by addition of brine (2 mL), diluted with 20 mL of ethyl acetate, and stirred for additional 15 min. The mixture was filtered through CELITE®, and the filter cake was washed with an additional 25 mL of ethyl acetate. The combined organic filtrates were dried over sodium sulfate, filtered again, and the solvent was evaporated. The crude product was used without further purification in the next step. ¹H NMR (400 MHz, Methanol-d₄) δ 8.13-7.97 (m, 1H), 7.79 (dd, 1H), 4.76 (d, 1H), 4.65 (d, 1H), 4.42 (dd, 1H), 4.07 (s, 3H), 3.85 (dt, 1H), 3.70 (dd, 1H), 2.55 (s, 3H).

Step ii. 8,9-Difluoro-6-methoxy-N-methyl-2,4-dihydro-1H-pyrano[3,4-c]isoquinolin-1-amine obtained as described above (210 mg, 0.75 mmol) in 5 mL of dichloromethane at 0° C. was treated with triethylamine (0.21 mL, 1.5 mmol), followed by tert-butoxycarbonyl tert-butyl carbonate (180 mg, 0.82 mmol) in dichloromethane (5 mL). After the addition was complete, the reaction was continued overnight allowing it to warm to room temperature. The reaction mixture was diluted with 30 mL of dichloromethane, washed with 0.5% HCl (20 mL), then with 5%. Sodium bicarbonate (20 mL), water (20 mL), and brine (20 mL). The organic extract was dried on sodium sulfate, filtered, concentrated and the product purified by flash chromatography (Silicagel, EtOAc/hexanes 0-15%) to afford racemic tert-butyl N-(8,9-difluoro-6-methoxy-2,4-dihydro-1H-pyrano[3,4-c]isoquinolin-1-yl)-N-methyl-carbamate (XII in Scheme 4, 226 mg, 78% yield over two steps). LCMS m/z 381.3 [M+H]⁺, RT=1.18 min (Method B); ¹H NMR (400 MHz, Chloroform-d) δ 7.96 (ddd, 1H), 7.56 (ddd, 1H), 5.49 (p, 1H), 5.25 (s)*, 4.81 (d, 1H), 4.65 (dd, 1H), 4.30-4.18 (m, 1H), 4.07 (s, 3H), 3.96 (ddd, 1H), 2.64 (d, 3H), 1.61 (s)*, 1.53 (s, 6H). “*” denotes signals of minor carbamate rotamer.

### tert-Butyl N-(8,9-Difluoro-4-hydroxy-6-methoxy-2,4-dihydro-1H1-pyrano[3,4-c]isoquinolin-1-yl)-N-methyl-carbamate (XIIIa)

tert-Butyl N-(8,9-Difluoro-6-methoxy-2,4-dihydro-1H-pyrano[3,4-c]isoquinolin-1-yl)-N-methyl-carbamate (XII, 100 mg, 0.26 mmol) in 5 mL of carbon tetracloride was treated with 1-bromopyrrolidine-2,5-dione (47 mg, 0.26 mmol) and benzoyl benzenecarboperoxoate (3 mg, 0.01 mmol) at 80° C. for 1 h. The reaction mixture was filtered, and solvent was evaporated. The residue was redissolved in a THF/water 1:1 v/v mixture (12 mL) and treated with 1 mL of a 1 M NaOH solution and stirred at 75° C. for 1 h. The reaction mixture was cooled to room temperature and treated with 2 M HCl, followed by saturated sodium bicarbonate to pH ˜6, and extracted with EtOAc. The organic extracts were dried on sodium sulfate, filtered and the solvent was evaporated. The product was isolated by flash-chromatography (Silicagel, EtOAc/Hexanes 0-55%) to afford tert-butyl N-(8,9-difluoro-4-hydroxy-6-methoxy-2,4-dihydro-1H-pyrano[3,4-c]isoquinolin-1-yl)-N-methyl-carbamate as a mixture of anomers (XIIIa in Scheme 4, 66 mg, 63% yield). LCMS m/z 397.3 [M+H]⁺, RT=1.02 min (minor anomer: RT=1.01 min) (Method B). ¹H NMR (400 MHz, Chloroform-d) δ 8.14-7.90 (m, 1H), 7.78-7.65 (m, 1H), 5.99 (s, 1H), 5.49 (d, 1H), 4.61 (td, 1H), 4.15 (s, 3H), 4.06 (d, 1H), 2.67-2.51 (m, 3H), 1.53 (s, 9H). NOTE: signals of the major anomer are reported.

### tert-Butyl N-(8,9-Difluoro-6-methoxy-4-oxo-1,2-dihydropyrano[3,4-c]isoquinolin-1-yl)-N-methyl-carbamate (XIIIb)

tert-Butyl N-(8,9-Difluoro-6-methoxy-2,4-dihydro-1H-pyrano[3,4-c]isoquinolin-1-yl)-N-methyl-carbamate (XII, 95 mg, 0.25 mmol) in 5 mL of dichloromethane was treated with pyridinium chlorochromate (323 mg, 1.5 mmol) and the reaction was stirred at 55° C. for 48 hours The reaction mixture was adsorbed directly on Silicagel and the product was purified by flash chromatography to afford tert-butyl N-(8,9-difluoro-6-methoxy-4-oxo-1,2-dihydropyrano[3,4-c]isoquinolin-1-yl)-N-methyl-carbamate (XIIIb in Scheme 4, 38 mg, 39% yield). LCMS m/z 395.2 [M+H], RT=1.01 min (Method B). ¹H NMR (400 MHz, Chloroform-d) δ 8.12 (dd, 1H), 7.98 (dd, 1H), 5.98 (d, 1H), 4.82 (dd, 1H), 4.72 (dd, 1H), 4.26 (s, 3H), 2.69 (s, 3H), 1.55 (s, 9H).

### 3-(3-Chloro-4-fluorophenyl)-1-(8,9-difluoro-4-hydroxy-6-oxo-1,4,5,6-tetrahydro-2H1-pyrano[3,4-c]isoquinolin-1-yl)-1-methylurea (Compound 151)

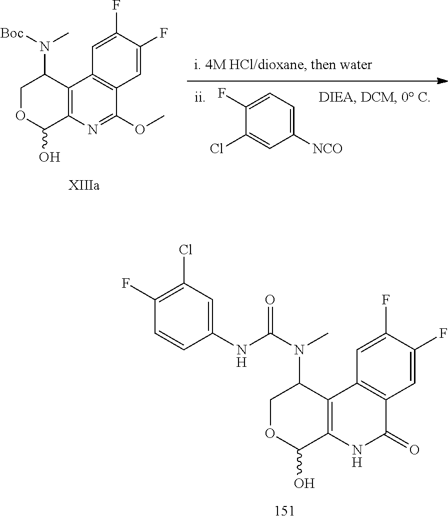

Step i. tert-Butyl N-(8,9-Difluoro-4-hydroxy-6-methoxy-2,4-dihydro-1H-pyrano[3,4-c]isoquinolin-1-yl)-N-methyl-carbamate (XIIIa, 26 mg, 0.07 mmol) was treated with 4 M hydrogen chloride (2 mL, 8.00 mmol) in dioxane for 1 h at room temperature. Water (0.4 mL, 22.2 mmol) was added dropwise and the reaction was continued for 16 h. The volatiles were evaporated, and the residue was azeotroped with toluene, then dried under high vacuum for 1 h, prior to use without further purification in the next step. LCMS m/z 283.1 [M+H]⁺, RT=0.45 min (minor anomer: RT=0.50 min) (Method B).

Step ii. Diisopropylethylamine (29 μL, 0.16 mmol) was added to a precooled (ice bath) mixture of 8,9-difluoro-4-hydroxy-1-(methylamino)-1,2,4,5-tetrahydropyrano[3,4-c]isoquinolin-6-one hydrochloride salt (18.5 mg, 0.07 mmol), obtained as described above, and 2-chloro-1-fluoro-4-isocyanato-benzene (8 μL, 0.06 mmol) in 1 mL of dichloromethane, and the reaction mixture was stirred for 1 h, allowing the bath to warm to room temperature. The reaction mixture was diluted with 10 mL EtOAc and washed with 0.2 M HCl (5 mL) and 5% sodium carbonate (5 mL), then with water and with brine, and dried over sodium sulfate. The organic solution was filtered, and the solvent was evaporated. The product was triturated with ethyl acetate, filtered and dried overnight under high vacuum, to provide 3-(3-chloro-4-fluorophenyl)-1-(8,9-difluoro-4-hydroxy-6-oxo-1,4,5,6-tetrahydro-2H-pyrano[3,4-c]isoquinolin-1-yl)-1-methylurea as a mixture of diastereoisomers (13.4 mg, 36% yield). LCMS m/z found 454.1/456.1 [M+H]⁺; RT=4.00 min (Method A); ¹H NMR (400 MHz, Acetonitrile-d₃) δ 9.75 (s, 1H), 8.19-8.07 (m, 1H), 7.76 (dd, 1H), 7.66-7.50 (m, 1H), 7.43 (dddd, 1H), 7.29 (d, 1H), 7.19 (t, 1H), 5.75-5.70 (m, 1H), 5.56-5.45 (m, 2H), 5.14 (s, 1H), 4.40 (ddd, 1H), 4.20-4.03 (m, 1H), 4.03-3.84 (m, 1H), 3.84-3.67 (m, 1H), 2.87 (s, 1H), 2.81 (s, 2H).

### 3-(3-Chloro-4-Fluorophenyl)-1-(8,9-difluoro-4,6-dioxo-1,4,5,6-tetrahydro-2H-pyrano[3,4-c]isoquinolin-1-yl)-1-methylurea (Compound 152)

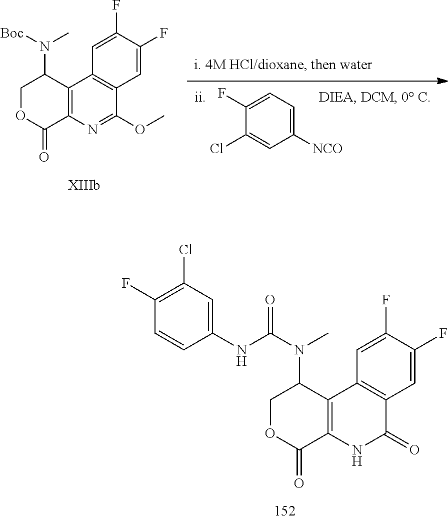

Racemic 3-(3-chloro-4-fluorophenyl)-1-(8,9-difluoro-4,6-dioxo-1,4,5,6-tetrahydro-2H-pyrano[3,4-c]isoquinolin-1-yl)-1-methylurea was synthesized by a two-step procedure in an analogous manner as described above for Compound 151, starting from racemic tert-butyl N-(8,9-difluoro-6-methoxy-4-oxo-1,2-dihydropyrano[3,4-c]isoquinolin-1-yl)-N-methyl-carbamate (XIIIb). LCMS m/z found 452.1/454.1 [M+H]⁺; RT=0.90 min (Method B); ¹H NMR (400 MHz, Acetonitrile-d₃; D₂O/CD₃OD) δ 8.23 (dd, 1H), 7.92 (dd, 1H), 7.70 (dd, 1H), 7.39 (ddd, 1H), 7.18 (t, 1H), 6.04 (d, 1H), 4.86 (dd, 1H), 4.70 (dd, 1H), 2.80 (s, 3H).

### (S)-1-(8,9-Difluoro-6-oxo-1,4,5,6-tetrahydro-2H-pyrano[3,4-c]isoquinolin-1-yl)-3-(4-fluoro-3-methylphenyl)-1-methylurea (Compound 69)

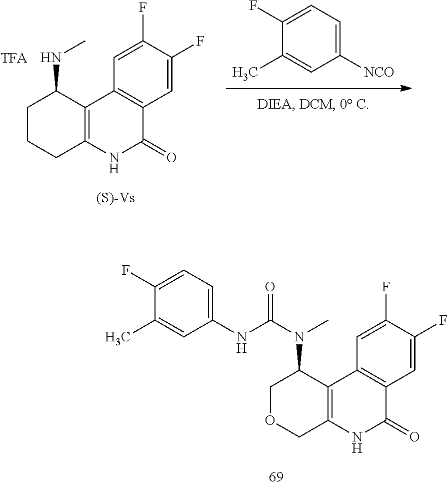

Enantiomerically pure (S)-1-(8,9-difluoro-6-oxo-1,4,5,6-tetrahydro-2H-pyrano[3,4-c]isoquinolin-1-yl)-3-(4-fluoro-3-methylphenyl)-1-methylurea was synthesized in an analogous manner as described above for Compound 41, starting from enantiomerically pure (S)-8,9-difluoro-1-(methylamino)-1,5-dihydro-2H-pyrano[3,4-c]isoquinolin-6(4H)-one mono-TFA salt (Vs) and 1-fluoro-4-isocyanato-2-methyl-benzene. LCMS m/z found 418 [M+H]⁺; RT=3.24 min (Method C); ¹H NMR (400 MHz, DMSO-d₆) δ 11.66 (s, 1H), 8.37 (s, 1H), 8.11 (dd, 1H), 7.50 (dd, 1H), 7.44 (dd, 1H), 7.39-7.30 (m, 1H), 7.05 (t, 1H), 5.42 (s, 1H), 4.58 (d, 1H), 4.47-4.37 (m, 1H), 4.04 (d, 1H), 3.93 (dd, 1H), 2.80 (s, 3H), 2.21 (d, 3H).

### (S)-3-(3-Chloro-4-methoxyphenyl)-1-(8,9-difluoro-6-oxo-1,4,5,6-tetrahydro-2H-pyrano[3,4-c]isoquinolin-1-yl)-1-methylurea (Compound 147)

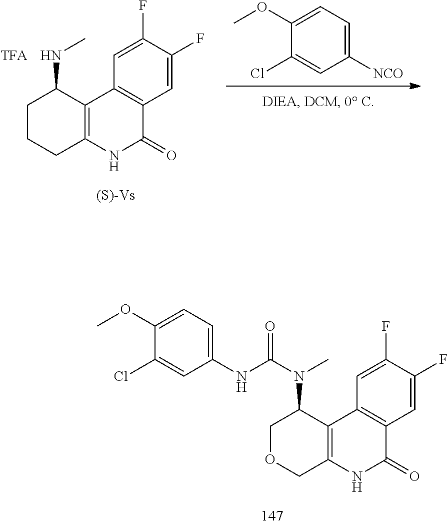

Enantiomerically pure (S)-3-(3-chloro-4-methoxyphenyl)-1-(8,9-difluoro-6-oxo-1,4,5,6-tetrahydro-2H-pyrano[3,4-c]isoquinolin-1-yl)-1-methylurea was synthesized in an analogous manner as described above for Compound 41, starting from enantiomerically pure (S)-8,9-difluoro-1-(methylamino)-1,5-dihydro-2H-pyrano[3,4-c]isoquinolin-6(4H)-one mono-TFA salt (Vs) and 2-chloro-4-isocyanato-1-methoxybenzene. LCMS m/z found 450.2/452.2 [M+H]⁺; RT=3.81 min (Method A); ¹H NMR (400 MHz, DMSO-d₆) δ 11.66 (s, 1H), 8.40 (s, 1H), 8.10 (dd, 1H), 7.69 (dd, 1H), 7.60-7.33 (m, 2H), 7.09 (d, 1H), 5.42 (s, 1H), 4.58 (d, 1H), 4.51-4.31 (m, 1H), 4.09-4.02 (m, 1H), 3.93 (dd, 1H), 3.82 (s, 3H), 2.80 (s, 3H).

### (S)-3-(3-Chloro-4-hydroxyphenyl)-1-(8,9-difluoro-6-oxo-1,4,5,6-tetrahydro-2H-pyrano[3,4-c]isoquinolin-1-yl)-1-methylurea (Compound 148)

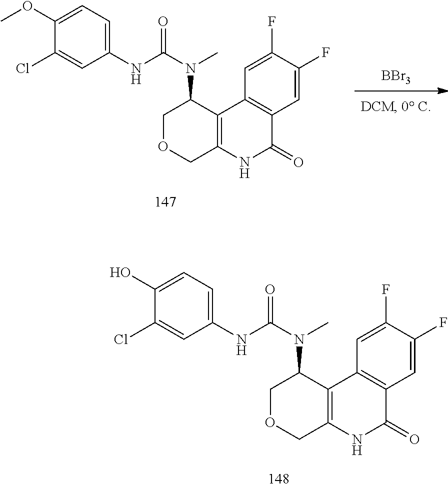

Tribromoborane (25 μL, 0.27 mmol) was added to a mixture of enantiomerically pure (S)-3-(3-chloro-4-methoxyphenyl)-1-(8,9-difluoro-6-oxo-1,4,5,6-tetrahydro-2H-pyrano[3,4-c]isoquinolin-1-yl)-1-methylurea (Compound 147.48 mg, 0.11 mmol) in 2.0 mL of dichloromethane that was precooled in an ice bath, and the reaction mixture was stirred for 1 h, then quenched by the slow addition of 1 mL of methanol, followed by a 5% solution of sodium bicarbonate. The reaction mixture was extracted with 30 mL of EtOAc and washed with 0.2 m HCl (10 mL) and a 5% solution of sodium bicarbonate (15 mL), then with water and with brine (10 mL each) and dried over sodium sulfate. The organic solution was filtered, and the solvent was evaporated. The product was triturated with ethyl acetate, filtered and dried overnight under high vacuum, to provide (S)-3-(3-chloro-4-hydroxyphenyl)-1-(8,9-difluoro-6-oxo-1,4,5,6-tetrahydro-2H-pyrano[3,4-c]isoquinolin-1-yl)-1-methylurea (28 mg, 60% yield). LCMS m/z found 436.2/438.1 [M+H]⁺; RT=2.97 min (Method A); ¹H NMR (400 MHz, DMSO-d₆) δ 11.65 (s, 1H), 9.76 (s, 1H), 8.29 (s, 1H), 8.10 (dd, 1H), 7.59-7.45 (m, 2H), 7.26 (dd, 1H), 6.92-6.78 (m, 1H), 5.41 (s, 1H), 4.58 (d, 1H), 4.46-4.37 (m, 1H), 4.12-3.99 (m, 1H), 3.92 (dd, 1H), 2.78 (s, 3H).

### (S)-3-(3-Cyano-4-fluorophenyl)-1-(8,9-difluoro-6-oxo-1,4,5,6-tetrahydro-2H-pyrano[3,4-c]isoquinolin-1-yl)-1-methylurea (Compound 70)

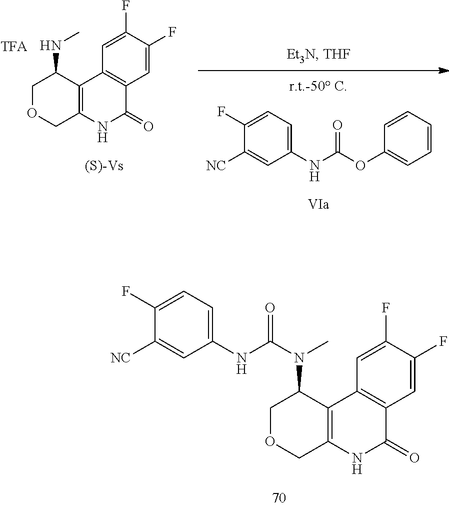 [SMILES: CN[C@@H]1COCc2[nH]c(=O)c3cc(F)c(F)cc3c21 ; N#Cc1cc(NC(=O)Oc2ccccc2)ccc1F ; CN(C(=O)Nc1ccc(F)c(C#N)c1)[C@@H]1COCc2[nH]c(=O)c3cc(F)c(F)cc3c21]

Triethylamine (26 uL, 0.19 mmol) was added to enantiomerically pure (S)-8,9-difluoro-1-(methylamino)-1,5-dihydro-2H-pyrano[3,4-c]isoquinolin-6(4H)-one mono-TFA salt (Vs) (29 mg, 0.08 mmol) in 1 mL anhydrous THF. Phenyl N-(3-cyano-4-fluoro-phenyl)carbamate (VIa, 19 mg, 0.08 mmol) was added, and the reaction mixture was stirred at room temperature for 5 min, then at 50° C. for 2 h. The reaction mixture was diluted with 30 mL EtOAc and washed with 0.2 M HCl (10 mL), then with a 5% NaHCO₃ aqueous solution (15 mL), and then with brine, and dried over sodium sulfate. The organic solution was filtered and the solvent was evaporated to a white solid, which was triturated from methanol and the product was collected by filtration, washed with methanol, then with 1:1 methanol/dichloromethane, then hexane, and dried overnight under high vacuum at 50° C. to provide enantiomerically pure (S)-3-(3-cyano-4-fluorophenyl)-1-(8,9-difluoro-6-oxo-1,4,5,6-tetrahydro-2H-pyrano[3,4-c]isoquinolin-1-yl)-1-methylurea (25.0 mg, 77.7%). LCMS m/z found 429.2 [M+H]⁺; RT=3.68 min, (Method A); ¹H NMR (400 MHz, DMSO-d₆) δ 11.67 (s, 1H), 8.77 (s, 1H), 8.10 (dd, 1H), 8.04 (dd, 1H), 7.89 (ddd, 1H), 7.52-7.40 (m, 2H), 5.41 (d, 1H), 4.59 (d, 1H), 4.47-4.38 (m, 1H), 4.12-4.05 (m, 1H), 3.93 (dd, 1H), 2.83 (s, 3H). Chiral analytical SFC: RT=4.83 min, Column: OD-10 analytical; 30% Methanol; Total flow: 3 g/min.

### (S)-3-(4-Chloro-3-fluorophenyl)-1-(8,9-difluoro-6-oxo-1,4,5,6-tetrahydro-2H-pyrano[3,4-c]isoquinolin-1-yl)-1-methylurea (Compound 141)

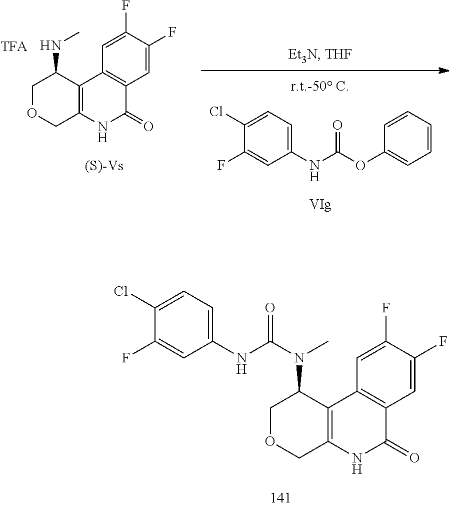 [SMILES: CN[C@@H]1COCc2[nH]c(=O)c3cc(F)c(F)cc3c21 ; O=C(Nc1ccc(Cl)c(F)c1)Oc1ccccc1 ; CN(C(=O)Nc1ccc(Cl)c(F)c1)[C@@H]1COCc2[nH]c(=O)c3cc(F)c(F)cc3c21]

Enantiomerically pure (S)-3-(4-chloro-3-fluorophenyl)-1-(8,9-difluoro-6-oxo-1,4,5,6-tetrahydro-2H-pyrano[3,4-c]isoquinolin-1-yl)-1-methylurea was synthesized in an analogous manner as described above, starting from enantiopure (S)-8,9-difluoro-1-(methylamino)-1,5-dihydro-2H-pyrano[3,4-c]isoquinolin-6(4H)-one mono-TFA salt (Vs) and phenyl N-(4-chloro-3-fluoro-phenyl)carbamate (VIg). LCMS m/z found 438.1 [M+H]⁺; RT=4.41 min (Method A); ¹H NMR (400 MHz, DMSO-d₆) δ 11.68 (s, 1H), 8.72 (s, 1H), 8.11 (dd, 1H), 7.74 (dd, 1H), 7.52-7.37 (m, 3H), 5.41 (s, 1H), 4.59 (d, 1H), 4.47-4.38 (m, 1H), 4.11-4.03 (m, 1H), 3.93 (dd, 1H), 2.83 (s, 3H).

### (S)-3-(4-Chloro-3-cyanophenyl)-1-(8,9-difluoro-6-oxo-1,4,5,6-tetrahydro-2H-pyrano[3,4-c]isoquinolin-1-yl)-1-methylurea (Compound 142)

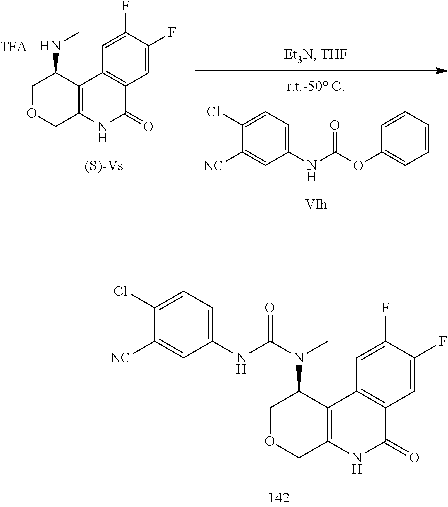 [SMILES: CN[C@@H]1COCc2[nH]c(=O)c3cc(F)c(F)cc3c21 ; N#Cc1cc(NC(=O)Oc2ccccc2)ccc1Cl ; CN(C(=O)Nc1ccc(Cl)c(C#N)c1)[C@@H]1COCc2[nH]c(=O)c3cc(F)c(F)cc3c21]

Enantiomerically pure (S)-3-(4-chloro-3-cyanophenyl)-1-(8,9-difluoro-6-oxo-1,4,5,6-tetrahydro-2H-pyrano[3,4-c]isoquinolin-1-yl)-1-methylurea was synthesized in an analogous manner as described above, starting from enantiopure (S)-8,9-difluoro-1-(methylamino)-1,5-dihydro-2H-pyrano[3,4-c]isoquinolin-6(4H)-one mono-TFA salt (Vs) and phenyl N-(4-chloro-3-cyano-phenyl)carbamate (VIh). LCMS m/z found 445.2/447.2 [M+H]⁺; RT=4.08 min (Method A); ¹H NMR (400 MHz, DMSO-d₆) δ 11.68 (s, 1H), 8.86 (s, 1H), 8.18-8.06 (m, 2H), 7.90 (dt, 1H), 7.66 (d, 1H), 7.45 (dd, 1H), 5.41 (s, 1H), 4.59 (d, 1H), 4.48-4.38 (m, 1H), 4.12-4.04 (m, 1H), 3.94 (dd, 1H), 2.84 (s, 3H).

### (S)-3-(3,4-Dichlorophenyl)-1-(8,9-difluoro-6-oxo-1,4,5,6-tetrahydro-2H-pyrano[3,4-c]isoquinolin-1-yl)-1-methylurea (Compound 143)

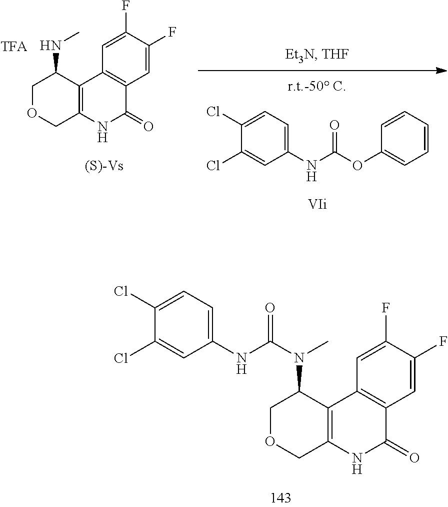 [SMILES: CN[C@@H]1COCc2[nH]c(=O)c3cc(F)c(F)cc3c21 ; O=C(Nc1ccc(Cl)c(Cl)c1)Oc1ccccc1 ; CN(C(=O)Nc1ccc(Cl)c(Cl)c1)[C@@H]1COCc2[nH]c(=O)c3cc(F)c(F)cc3c21]

Enantiomerically pure (S)-3-(3,4-dichlorophenyl)-1-(8,9-difluoro-6-oxo-1,4,5,6-tetrahydro-2H-pyrano[3,4-c]isoquinolin-1-yl)-1-methylurea was synthesized in an analogous manner as described above, starting from enantiopure (S)-8,9-difluoro-1-(methylamino)-1,5-dihydro-2H-pyrano[3,4-c]isoquinolin-6(4H)-one mono-TFA salt (Vs) and 1,2-dichloro-4-isocyanato-benzene (VIi). LCMS m/z found 454.1/456.2 [M+H]⁺; RT=4.71 min (Method A); ¹H NMR (400 MHz, DMSO-d₆) δ 11.67 (s, 1H), 8.69 (s, 1H), 8.11 (dd, 1H), 7.94 (d, 1H), 7.61-7.49 (m, 2H), 7.46 (dd, 1H), 5.41 (s, 1H), 4.59 (d, 1H), 4.47-4.38 (m, 1H), 4.11-4.01 (m, 1H), 3.93 (dd, 1H), 2.82 (s, 3H).

### (S)-1-(8,9-Difluoro-6-oxo-1,4,5,6-tetrahydro-2H-pyrano[3,4-c]isoquinolin-1-yl)-1-methyl-3-(1-(trifluoromethyl)cyclopropyl)urea (Compound 145)

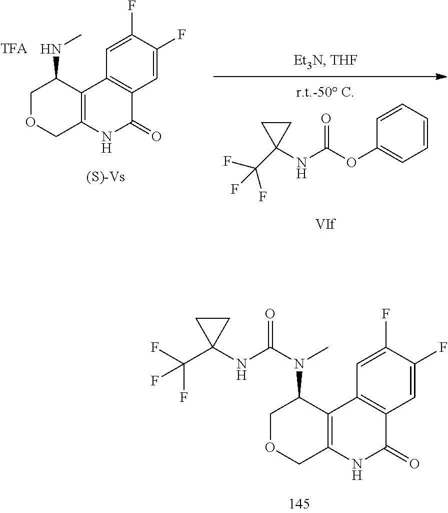 [SMILES: CN[C@@H]1COCc2[nH]c(=O)c3cc(F)c(F)cc3c21 ; O=C(NC1(C(F)(F)F)CC1)Oc1ccccc1 ; CN(C(=O)NC1(C(F)(F)F)CC1)[C@@H]1COCc2[nH]c(=O)c3cc(F)c(F)cc3c21]

Enantiomerically pure (S)-1-(8,9-difluoro-6-oxo-1,4,5,6-tetrahydro-2H-pyrano[3,4-c]isoquinolin-1-yl)-1-methyl-3-(1-(trifluoromethyl)cyclopropyl)urea was synthesized in an analogous manner as described above, starting from enantiopure (S)-8,9-difluoro-1-(methylamino)-1,5-dihydro-2H-pyrano[3,4-c]isoquinolin-6(4H)-one mono-TFA salt (Vs) and phenyl N-[1-(trifluoromethyl) cyclopropyl]carbamate (VIf). LCMS m/z found 418.35 [M+H]⁺; RT=3.10 min (Method A); ¹H NMR (400 MHz, DMSO-d₆) δ 11.48 (s, 1H), 7.95 (dd, 1H), 7.34 (dd, 1H), 7.25-7.20 (m, 1H), 5.23 (s, 1H), 4.41 (d, 1H), 4.30-4.21 (m, 1H), 3.86-3.71 (m, 2H), 2.50 (s, 3H), 1.19-1.06 (m, 2H), 1.02 (d, 1H), 0.92 (s, 1H).

### (S)—N-(8,9-Difluoro-6-oxo-1,4,5,6-tetrahydro-2H-pyrano[3,4-c]isoquinolin-1-yl)-N-methylisoindoline-2-carboxamide (Compound 218)

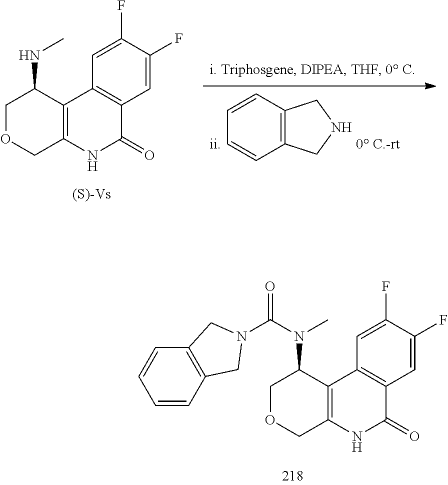 [SMILES: CN[C@@H]1COCc2[nH]c(=O)c3cc(F)c(F)cc3c21 ; c1ccc2c(c1)CNC2 ; CN(C(=O)N1Cc2ccccc2C1)[C@@H]1COCc2[nH]c(=O)c3cc(F)c(F)cc3c21]

To a stirred solution of 30 mg (0.113 mmol, 1 eq) of (S)-8,9-difluoro-1-(methylamino)-1,5-dihydro-2H-pyrano[3,4-c]isoquinolin-6(4H)-one, (S)-Vs in 1 mL of THE was added 0.04 ml DIPEA (0.28 mmol, 2.5 eq) and 20 mg (0.068 mmol, 0.6 eq) of triphosgene at 0° C. and the reaction mixture was stirred at the same temperature for 30 min. Isoindoline (13.4 mg, 0.113 mmol, 1 eq) was added and the reaction was continued for 4 h. The reaction mixture was poured into water (20 mL) and extracted with ethyl acetate (2×10 mL). Combined organic layers were washed with water (10 mL), dried over anhydrous sodium sulfate and concentrated under reduced pressure. The product was purified by column chromatography (Silicagel, isocratic 60% ethyl acetate in petroleum ether) to afford 10 mg (22% yield) of (S)—N-(8,9-difluoro-6-oxo-1,4,5,6-tetrahydro-2H-pyrano[3,4-c]isoquinolin-1-yl)-N-methylisoindoline-2-carboxamide as an off white solid. LCMS m/z found 412.3 [M+H]⁺; RT=7.24 min (Method A); ¹H NMR (400 MHz, DMSO-d₆) δ 11.64 (s, 1H), 8.13-8.08 (m, 1H), 7.77-7.72 (q, 1H), 7.34-7.27 (m, 4H) 5.14 (s, 1H), 4.82 (d, 2H), 4.71 (d, 2H), 4.58 (d, 1H), 4.42 (d, 1H), 4.23 (d, 1H), 3.96-3.92 (m, 1H), 2.79 (s, 3H).

### (S)-5-Chloro-N-(8,9-difluoro-6-oxo-1,4,5,6-tetrahydro-2H-pyrano[3,4-c]isoquinolin-1-yl)-N-methylisoindoline-2-carboxamide (Compound 219)

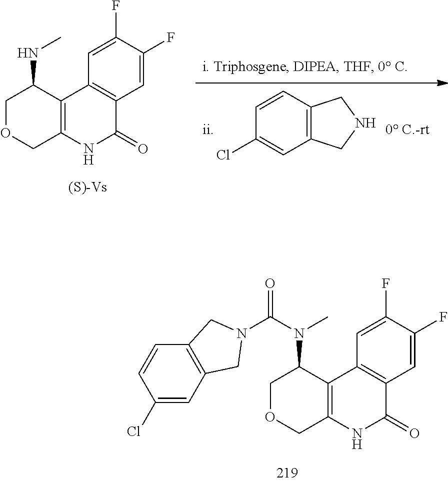 [SMILES: CN[C@@H]1COCc2[nH]c(=O)c3cc(F)c(F)cc3c21 ; Clc1ccc2c(c1)CNC2 ; CN(C(=O)N1Cc2ccc(Cl)cc2C1)[C@@H]1COCc2[nH]c(=O)c3cc(F)c(F)cc3c21]

Enantiomerically pure (S)-5-chloro-N-(8,9-difluoro-6-oxo-1,4,5,6-tetrahydro-2H-pyrano[3,4-c]isoquinolin-1-yl)-N-methylisoindoline-2-carboxamide was synthesized in an analogous manner as described above, starting from enantiopure (S)-8,9-difluoro-1-(methylamino)-1,5-dihydro-2H-pyrano[3,4-c]isoquinolin-6(4H)-one (Vs) and 5-chloroisoindoline. LCMS m/z found 446.3/448.3 [M+H]⁺; RT=7.54 min (Method A); ¹H NMR (400 MHz, DMSO-d₆) δ 11.64 (s, 1H), 8.1 (t, 1H), 7.76-7.71 (q, 1H), 7.43 (s, 1H), 7.37-7.32 (m, 2H), 5.13 (s, 1H), 4.83-4.66 (m, 4H), 4.57 (d, 1H), 4.42 (d, 1H), 4.22 (d, 1H), 3.95-3.91 (m, 1H), 2.77 (s, 3H).

### (S)-5-Bromo-N-(8,9-difluoro-6-oxo-1,4,5,6-tetrahydro-2H-pyrano[3,4-c]isoquinolin-1-yl)-N-methylisoindoline-2-carboxamide (Compound 220)

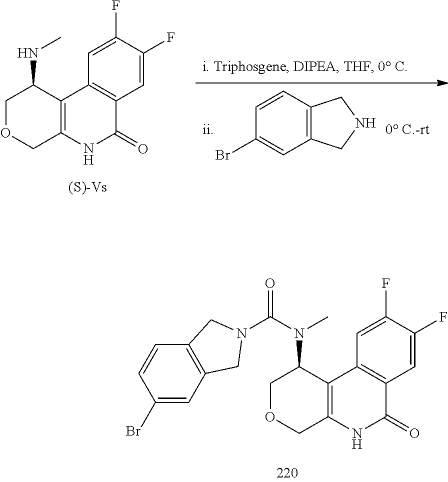 [SMILES: CN[C@@H]1COCc2[nH]c(=O)c3cc(F)c(F)cc3c21 ; Brc1ccc2c(c1)CNC2 ; CN(C(=O)N1Cc2ccc(Br)cc2C1)[C@@H]1COCc2[nH]c(=O)c3cc(F)c(F)cc3c21]

Enantiomerically pure (S)-5-bromo-N-(8,9-difluoro-6-oxo-1,4,5,6-tetrahydro-2H-pyrano[3,4-c]isoquinolin-1-yl)-N-methylisoindoline-2-carboxamide was synthesized in an analogous manner as described above, starting from enantiopure (S)-8,9-difluoro-1-(methylamino)-1,5-dihydro-2H-pyrano[3,4-c]isoquinolin-6(4H)-one (Vs) and 5-bromoisoindoline. LCMS m/z found 490.2 [M+H]⁺; RT=7.57 min (Method A); ¹H NMR (400 MHz, DMSO-d₆) δ 11.64 (s, 1H), 8.1 (t, 1H), 7.77-7.72 (q, 1H), 7.56 (s, 1H), 7.46 (d, 1H), 7.30 (d, 1H), 5.13 (s, 1H), 4.83-4.55 (m, 5H), 4.42 (d, 1H), 4.22 (d, 1H), 3.95-3.91 (m, 1H), 2.77 (s, 3H).

### (S)—N-(8,9-Difluoro-6-oxo-1,4,5,6-tetrahydro-2H-pyrano[3,4-c]isoquinolin-1-yl)-5-fluoro-N-methylisoindoline-2-carboxamide (Compound 221)

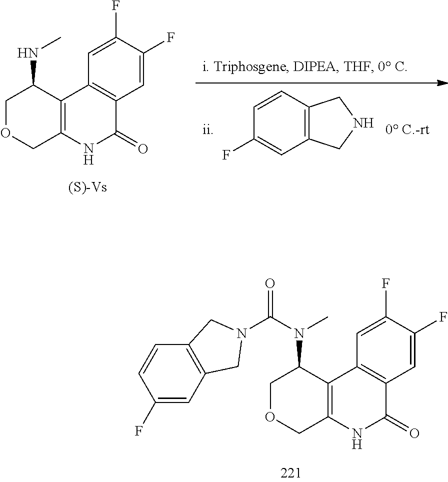 [SMILES: CN[C@@H]1COCc2[nH]c(=O)c3cc(F)c(F)cc3c21 ; Fc1ccc2c(c1)CNC2 ; CN(C(=O)N1Cc2ccc(F)cc2C1)[C@@H]1COCc2[nH]c(=O)c3cc(F)c(F)cc3c21]

Enantiomerically pure (S)—N-(8,9-difluoro-6-oxo-1,4,5,6-tetrahydro-2H-pyrano[3,4-c]isoquinolin-1-yl)-5-fluoro-N-methylisoindoline-2-carboxamide was synthesized in an analogous manner as described above, starting from enantiopure (S)-8,9-difluoro-1-(methylamino)-1,5-dihydro-2H-pyrano[3,4-c]isoquinolin-6(4H)-one (Vs) and 5-fluoroisoindoline. LCMS m/z found 430.2 [M+H]⁺; RT=6.39 min (Method A); ¹H NMR (400 MHz, DMSO-d₆) δ 11.64 (s, 1H), 8.1 (t, 1H), 7.76-7.71 (q, 1H), 7.36 (t, 1H), 7.18 (d, 1H), 7.08 (t, 1H), 5.13 (s, 1H), 4.83-4.66 (m, 4H), 4.57 (d, 1H), 4.42 (d, 1H), 4.22 (d, 1H), 3.95-3.91 (m, 1H), 2.77 (s, 3H).

### (S)—N-(8,9-Difluoro-6-oxo-1,4,5,6-tetrahydro-2H-pyrano[3,4-c]isoquinolin-1-yl)-N-methyl-5-(trifluoromethyl)isoindoline-2-carboxamide (Compound 230)

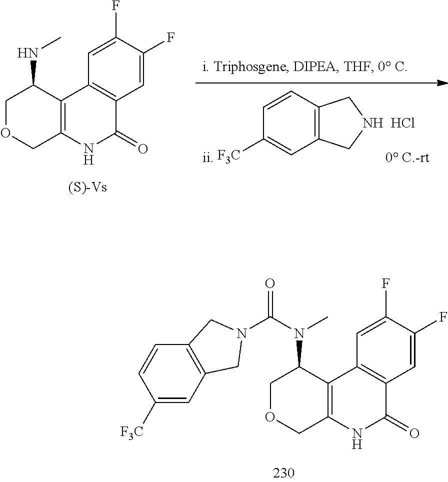 [SMILES: CN[C@@H]1COCc2[nH]c(=O)c3cc(F)c(F)cc3c21 ; FC(F)(F)c1ccc2c(c1)CNC2 ; Cl ; CN(C(=O)N1Cc2ccc(C(F)(F)F)cc2C1)[C@@H]1COCc2[nH]c(=O)c3cc(F)c(F)cc3c21]

Enantiomerically pure (S)—N-(8,9-difluoro-6-oxo-1,4,5,6-tetrahydro-2H-pyrano[3,4-c]isoquinolin-1-yl)-N-methyl-5-(trifluoromethyl)isoindoline-2-carboxamide was synthesized in an analogous manner as described above, except for employing dimethyl formamide as the solvent, starting from enantiopure (S)-8,9-difluoro-1-(methylamino)-1,5-dihydro-2H-pyrano[3,4-c]isoquinolin-6(4H)-one (Vs) and 5-(trifluoromethyl)isoindoline hydrochloride. LCMS m/z found 480.2 [M+H]⁺; RT=4.72 min (Method A); ¹H NMR (400 MHz, DMSO-d₆) δ 11.64 (s, 1H), 8.13 (t, 1H), 7.78-7.73 (m, 2H), 7.66 (d, 1H), 7.57 (d, 1H), 5.14 (s, 1H), 4.90-4.76 (q, 4H), 4.60 (d, 1H), 4.44 (d, 1H), 4.25 (d, 1H), 3.95 (d, 1H), 2.79 (s, 3H).

### 3-(3-Cyano-4-fluorophenyl)-1-(8,9-difluoro-6-oxo-1,4,5,6-tetrahydro-2H-pyrano[3,4-c]isoquinolin-1-yl)-1-methylurea (Compound 106)

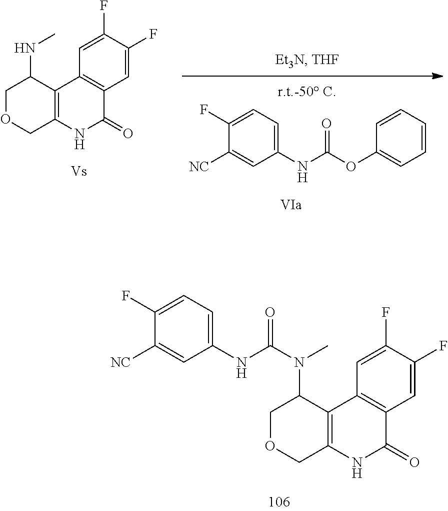 [SMILES: CNC1COCc2[nH]c(=O)c3cc(F)c(F)cc3c21 ; N#Cc1cc(NC(=O)Oc2ccccc2)ccc1F ; CN(C(=O)Nc1ccc(F)c(C#N)c1)C1COCc2[nH]c(=O)c3cc(F)c(F)cc3c21]

Racemic 3-(3-cyano-4-fluorophenyl)-1-(8,9-difluoro-6-oxo-1,4,5,6-tetrahydro-2H-pyrano[3,4-c]isoquinolin-1-yl)-1-methylurea was synthesized in an analogous manner as described above, starting from racemic 8,9-difluoro-1-(methylamino)-1,2,4,5-tetrahydropyrano[3,4-c]isoquinolin-6-one (Vs). LCMS m/z found 429.3 [M+H]⁺; RT=3.68 min, (Method A); ¹H NMR (400 MHz, DMSO-d₆) δ 11.68 (s, 1H), 8.77 (s, 1H), 8.11 (dd, 1H), 8.04 (dd, 1H), 7.89 (ddd, 1H), 7.52-7.41 (m, 2H), 5.41 (d, 1H), 4.59 (d, 1H), 4.43 (dd, 1H), 4.12-3.97 (m, 1H), 3.93 (dd, 1H), 2.83 (s, 3H).

### 1-(8,9-Difluoro-6-oxo-1,4,5,6-tetrahydro-2H-pyrano[3,4-c]isoquinolin-1-yl)-3-(3-(difluoromethyl)-4-fluorophenyl)-1-methylurea (Compounds 125 and 126)

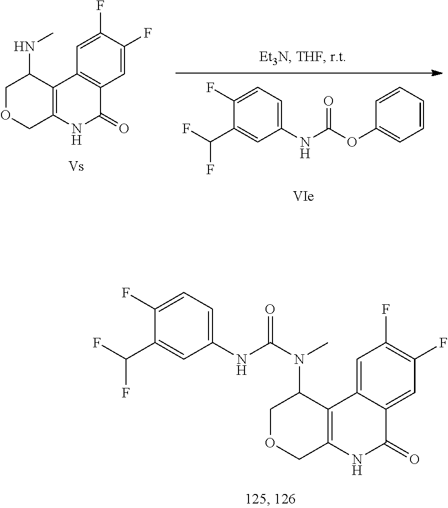 [SMILES: CNC1COCc2[nH]c(=O)c3cc(F)c(F)cc3c21 ; O=C(Nc1ccc(F)c(C(F)F)c1)Oc1ccccc1 ; CN(C(=O)Nc1ccc(F)c(C(F)F)c1)C1COCc2[nH]c(=O)c3cc(F)c(F)cc3c21]

1-(8,9-Difluoro-6-oxo-1,4,5,6-tetrahydro-2H-pyrano[3,4-c]isoquinolin-1-yl)-3-(3-(difluoromethyl)-4-fluorophenyl)-1-methylurea was synthesized in an analogous manner as described above, starting from 8,9-difluoro-1-(methylamino)-1,2,4,5-tetrahydropyrano[3,4-c]isoquinolin-6-one (Vs) and 3-(difluoromethyl)-4-fluorophenylcarbamate (VIe). The enantiomers were subsequently separated by preparative SFC: Method isocratic, Mobile phase MeOH: CO₂—40:60. Column: Chiralpak IG (30×250 mm), 5μ, flow rate: 90 g/min.

(R)-1-(8,9-Difluoro-6-oxo-1,4,5,6-tetrahydro-2H-pyrano[3,4-c]isoquinolin-1-yl)-3-(3-(difluoromethyl)-4-fluorophenyl)-1-methylurea: Enantiomer I (Compound 125): LCMS: m/z found 454.3 [M+H]⁺, RT=4.00 min, (Method A); ¹H NMR (400 MHz, DMSO-d₆): δ 11.62 (br s, 1H), 8.64 (s, 1H), 8.11-8.06 (m, 1H), 7.89-7.86 (m, 1H), 7.75-7.71 (m, 1H), 7.46-7.41 (m, 1H), 7.32-7.27 (m, 1H), 7.21 (t, 1H), 5.41 (s, 1H), 4.57 (d, 1H), 4.41 (d, 1H), 4.06 (d, 1H), 3.95-3.91 (m, 1H), 2.82 (s, 3H); Chiral analytical SFC: RT=0.96 min, Column CHIRALPAK IG-3 (4.6×150 mm) 3 um, 35% Methanol, Flow rate: 3.0 g/min.

(S)-1-(8,9-Difluoro-6-oxo-1,4,5,6-tetrahydro-2H-pyrano[3,4-c]isoquinolin-1-yl)-3-(3-(difluoromethyl)-4-fluorophenyl)-1-methylurea: Enantiomer II (Compound 126): LCMS: m/z found 454.3 [M+H]⁺, RT=4.00 min, (Method A); ¹H NMR (400 MHz, DMSO-d₆): δ 11.62 (br s, 1H), 8.64 (s, 1H), 8.11-8.06 (m, 1H), 7.89-7.86 (m, 1H), 7.75-7.71 (m, 1H), 7.46-7.41 (m, 1H), 7.32-7.27 (m, 1H), 7.21 (t, 1H), 5.41 (s, 1H), 4.57 (d, 1H), 4.41 (d, 1H), 4.06 (d, 1H), 3.95-3.91 (m, 1H), 2.82 (s, 3H); Chiral analytical SFC: RT=7.24 min, Column CHIRALPAK IG-3 (4.6×150 mm) 3 um, 35% Methanol, Flow rate: 3.0 g/min. Enantiomer II (Compound 126) was also prepared independently in an analogous manner as described above, starting from enantiomerically pure (S)-8,9-difluoro-1-(methylamino)-1,2,4,5-tetrahydropyrano[3,4-c]isoquinolin-6-one ((S)-Vs) and 3-(difluoromethyl)-4-fluorophenylcarbamate (VIe), in 62% yield after recrystallization from ethyl acetate.

### 1-(8,9-Difluoro-6-oxo-1,4,5,6-tetrahydro-2H-pyrano[3,4-c]isoquinolin-1-yl)-1-methyl-3-phenylurea (Compounds 101 and 102)

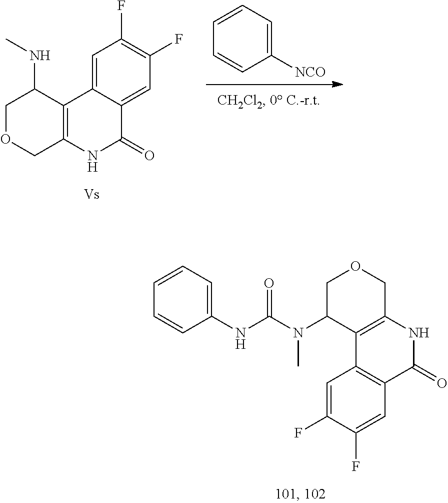

1-(8,9-Difluoro-6-oxo-1,4,5,6-tetrahydro-2H-pyrano[3,4-c]isoquinolin-1-yl)-1-methyl-3-phenylurea was synthesized in an analogous manner as described above for Compound 24, from racemic 8,9-difluoro-1-(methylamino)-1,2,4,5-tetrahydropyrano[3,4-c]isoquinolin-6-one (Vs) and isocyanatobenzene. The enantiomers were subsequently separated by preparative SFC: Method isocratic, Mobile phase MeOH: CO₂—30:70. Column: Chiralpak IC (30×250 mm), 5μ, flow rate: 90 g/min.

Enantiomer I (Compound 101): LCMS: m/z found 386.2 [M+H]⁺, RT=3.46 min, (Method A); ¹H NMR (400 MHz, DMSO-d₆) δ 11.61 (br s, 1H), 8.38 (br s, 1H), 8.12-8.07 (m, 1H), 7.55-7.49 (m, 3H), 7.29-7.25 (m, 2H), 7.00-6.96 (m, 1H), 5.43 (s, 1H), 4.58 (d, 1H), 4.41 (d, 1H), 4.05 (d, 1H), 3.95-3.91 (m, 1H), 2.82 (s, 3H); Chiral analytical SFC: RT=3.55 min, Column: CHIRALPAK IC-3 (4.6×150 mm) 3 um, 25% methanol, Flow rate: 3 g/min.

Enantiomer II (Compound 102): LCMS: m/z found 386.2 [M+H]⁺, RT=3.44 min, (Method A); ¹H NMR (400 MHz, DMSO-d₆) δ 11.61 (br s, 1H), 8.38 (br s, 1H), 8.12-8.07 (m, 1H), 7.55-7.49 (m, 3H), 7.29-7.25 (m, 2H), 7.00-6.96 (m, 1H), 5.43 (s, 1H), 4.58 (d, 1H), 4.41 (d, 1H), 4.05 (d, 1H), 3.95-3.91 (m, 1H), 2.82 (s, 3H); Chiral analytical SFC: RT=5.79 min, Column: CHIRALPAK IC-3 (4.6×150 mm) 3 um, 25% methanol, Flow rate: 3 g/min.

### 1-(8,9-Difluoro-6-oxo-1,4,5,6-tetrahydro-2H-pyrano[3,4-c]isoquinolin-1-yl)-3-(4-fluorophenyl)-1-methylurea (Compounds 103 and 104)

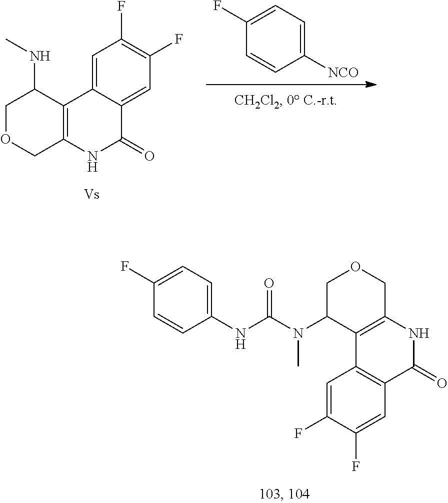

1-(8,9-Difluoro-6-oxo-1,4,5,6-tetrahydro-2H-pyrano[3,4-c]isoquinolin-1-yl)-3-(4-fluorophenyl)-1-methylurea was synthesized in an analogous manner as described above, from 8,9-difluoro-1-(methylamino)-1,2,4,5-tetrahydropyrano[3,4-c]isoquinolin-6-one (Vs) and 1-fluoro-4-isocyanatobenzene. The enantiomers were subsequently separated by preparative SFC: Method isocratic, Mobile phase MeOH: CO₂—30:70. Column: Chiralpak IC (30×250 mm), 5μ, flow rate: 100 g/min.

Enantiomer I (Compound 103): LCMS: m/z found 404.2 [M+H]⁺, RT=3.59 min, (Method A); ¹H NMR (400 MHz, DMSO-d₆) δ 11.61 (br s, 1H), 8.43 (br s, 1H), 8.13-8.08 (m, 1H), 7.56-7.48 (m, 3H), 7.14-7.09 (m, 2H), 5.43 (s, 1H), 4.58 (d, 1H), 4.42 (d, 1H), 4.05 (d, 1H), 3.95-3.91 (m, 1H), 2.81 (s, 3H); Chiral analytical SFC: RT=3.60 min, Column: CHIRALPAK IC-3 (4.6×150 mm) 3 μm, 20% methanol, Flow rate: 3 g/min.

Enantiomer II (Compound 104): LCMS: m/z found 404.2 [M+H]⁺, RT=3.60 min, (Method A); ¹H NMR (400 MHz, DMSO-d₆) δ 11.61 (br s, 1H), 8.43 (br s, 1H), 8.13-8.08 (m, 1H), 7.56-7.48 (m, 3H), 7.14-7.09 (m, 2H), 5.43 (s, 1H), 4.58 (d, 1H), 4.42 (d, 1H), 4.05 (d, 1H), 3.95-3.91 (m, 1H), 2.81 (s, 3H); Chiral analytical SFC: RT=5.55 min, Column: CHIRALPAK IC-3 (4.6×150 mm) 3 μm, 20% methanol, Flow rate: 3 g/min.

### 1-(8,9-Difluoro-6-oxo-1,4,5,6-tetrahydro-2H-pyrano[3,4-c]isoquinolin-1-yl)-3-(3,4-difluorophenyl)-1-methylurea (Compounds 117 and 118)

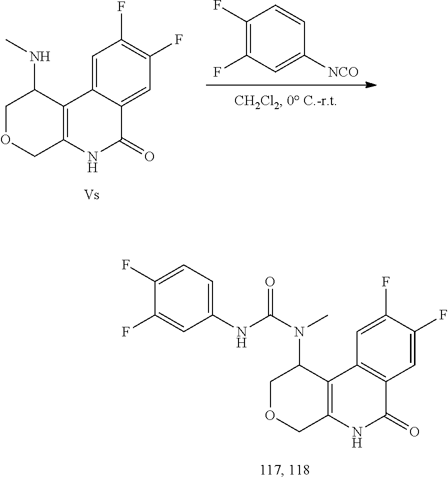

1-(8,9-Difluoro-6-oxo-1,4,5,6-tetrahydro-2H-pyrano[3,4-c]isoquinolin-1-yl)-3-(3,4-difluorophenyl)-1-methylurea was synthesized in an analogous manner as described above, from 8,9-difluoro-1-(methylamino)-1,2,4,5-tetrahydropyrano[3,4-c]isoquinolin-6-one (Vs) and 1,2-difluoro-4-isocyanatobenzene. The enantiomers were subsequently separated by preparative SFC: Method isocratic, Mobile phase MeOH: CO₂—20:80. Column: Chiralpak IC (30×250 mm), 5μ, flow rate: 90 g/min.

Enantiomer I (Compound 117): LCMS: m/z found 422.2 [M+H]⁺, RT=3.95 min, (Method A); ¹H NMR (400 MHz, DMSO-d₆): δ 11.61 (br s, 1H), 8.60 (br s, 1H), 8.13-8.08 (m, 1H), 7.74-7.68 (m, 1H), 7.50-7.44 (m, 1H), 7.36-7.31 (m, 2H), 5.41 (s, 1H), 4.58 (d, 1H), 4.42 (d, 1H), 4.05 (d, 1H), 3.95-3.91 (m, 1H), 2.82 (s, 3H); Chiral analytical SFC: RT=3.03 min, Column: CHIRALPAK IC-3 (4.6×150 mm) 3 μm, 20% methanol, Flow rate: 3 g/min.

Enantiomer II (Compound 118): LCMS: m/z found 422.3 [M+H]⁺, RT=4.95 min, (Method A); ¹H NMR (400 MHz, DMSO-d₆): δ 11.61 (br s, 1H), 8.60 (br s, 1H), 8.13-8.08 (m, 1H), 7.74-7.68 (m, 1H), 7.50-7.45 (m, 1H), 7.36-7.31 (m, 2H), 5.41 (s, 1H), 4.58 (d, 1H), 4.42 (d, 1H), 4.05 (d, 1H), 3.95-3.91 (m, 1H), 2.82 (s, 3H); HPLC: 98.95%, RT=10.65 min; Chiral analytical SFC: RT=4.49 min, Column: CHIRALPAK IC-3 (4.6×150 mm) 3 μm, 20% methanol, Flow rate: 3 g/min.

### 3-(3-Chlorophenyl)-1-(8,9-difluoro-6-oxo-1,4,5,6-tetrahydro-2H-pyrano[3,4-c]isoquinolin-1-yl)-1-methylurea (Compounds 119 and 120)

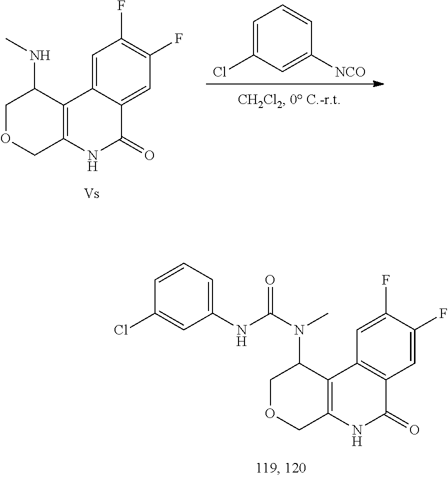

3-(3-Chlorophenyl)-1-(8,9-difluoro-6-oxo-1,4,5,6-tetrahydro-2H-pyrano[3,4-c]isoquinolin-1-yl)-1-methylurea was synthesized in an analogous manner as described above, from 8,9-difluoro-1-(methylamino)-1,2,4,5-tetrahydropyrano[3,4-c]isoquinolin-6-one (Vs) and 1-chloro-3-isocyanatobenzene. The enantiomers were subsequently separated by preparative SFC: Method isocratic, Mobile phase MeOH: CO₂—35:65, Column: Chiralpak IC (30×250 mm), 5μ, flow rate: 90 g/min.

Enantiomer I (Compound 119): LCMS: m/z found 420.2/422.2 [M+H]⁺, RT=4.16 min, (Method A); ¹H NMR (400 MHz, DMSO-d₆): δ 11.65 (br s, 1H), 8.57 (br s, 1H), 8.13-8.08 (m, 1H), 7.75-7.74 (m, 1H), 7.51-7.45 (m, 2H), 7.32-7.28 (m, 1H), 7.04-7.02 (m, 1H), 5.42 (s, 1H), 4.58 (d, 1H), 4.42 (d, 1H), 4.06 (d, 1H), 3.95-3.91 (m, 1H), 2.82 (s, 3H); Chiral SFC: RT=2.25 min, Column: CHIRALPAK IC-3 (4.6×150 mm) 3 μm, 30% methanol, Flow rate: 3 g/min.

Enantiomer II (Compound 120): LCMS: m/z found 420.2/422.2 [M+H]⁺, RT=4.16 min, (Method A); ¹H NMR (400 MHz, DMSO-d₆): δ 11.65 (br s, 1H), 8.57 (br s, 1H), 8.13-8.08 (m, 1H), 7.75-7.74 (m, 1H), 7.51-7.45 (m, 2H), 7.32-7.28 (m, 1H), 7.04-7.02 (m, 1H), 5.42 (s, 1H), 4.58 (d, 1H), 4.42 (d, 1H), 4.06 (d, 1H), 3.95-3.91 (m, 1H), 2.82 (s, 3H); Chiral SFC: RT=3.44 min, Column: CHIRALPAK IC-3 (4.6×150 mm) 3 μm, 30% methanol, Flow rate: 3 g/min.

### 1-(8,9-Difluoro-6-oxo-1,4,5,6-tetrahydro-2H-pyrano[3,4-c]isoquinolin-1-yl)-1-methyl-3-(3,4,5-trifluorophenyl)urea (Compounds 133 and 134)

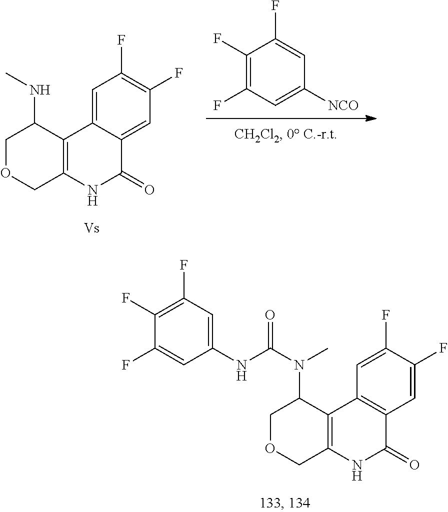

1-(8,9-Difluoro-6-oxo-1,4,5,6-tetrahydro-2H-pyrano[3,4-c]isoquinolin-1-yl)-1-methyl-3-(3,4,5-trifluorophenyl)urea was synthesized in an analogous manner as described above, from 8,9-difluoro-1-(methylamino)-1,2,4,5-tetrahydropyrano[3,4-c]isoquinolin-6-one (Vs) and 1,2,3-trifluoro-5-isocyanatobenzene. The enantiomers were subsequently separated by preparative SFC: Method isocratic, Mobile phase MeOH: CO₂—15:85, Column: (R, R) WHELK-01 (30×250 mm), 5μ, flow rate: 100 g/min.

Enantiomer I (Compound 133): LCMS: m/z found 440.2 [M+H]⁺, RT=4.37 min, (Method A); ¹H NMR (400 MHz, DMSO-d₆): δ 11.66 (br s, 1H), 8.74 (br s, 1H), 8.13-8.07 (m, 1H), 7.57-7.52 (m, 2H), 7.44-7.39 (m, 1H), 5.40 (s, 1H), 4.58 (d, 1H), 4.42 (d, 1H), 4.06 (d, 1H), 3.94-3.90 (m, 1H), 2.82 (s, 3H); Chiral analytical SFC: RT=3.88 min, Column: (R,R)WHELK-01 (4.6×150 mm) 3.5 μm, 20% methanol, Flow rate: 3 g/min.

Enantiomer II (Compound 134): LCMS: m/z found 440.3 [M+H]⁺, RT=4.37 min, (Method A); ¹H NMR (400 MHz, DMSO-d₆): δ 11.66 (br s, 1H), 8.74 (br s, 1H), 8.13-8.07 (m, 1H), 7.57-7.52 (m, 2H), 7.44-7.39 (m, 1H), 5.40 (s, 1H), 4.58 (d, 1H), 4.42 (d, 1H), 4.06 (d, 1H), 3.94-3.90 (m, 1H), 2.82 (s, 3H); Chiral analytical SFC: RT=4.56 min, Column: (R,R)WHELK-01 (4.6×150 mm) 3.5 μm, 20% methanol, Flow rate: 3 g/min.

### 3-(3-Chloro-4-fluorophenyl)-1-(8,9-difluoro-6-oxo-1,4,5,6-tetrahydro-2H-pyrano[3,4-c]isoquinolin-1-yl)-1-ethylurea (Compound 45)

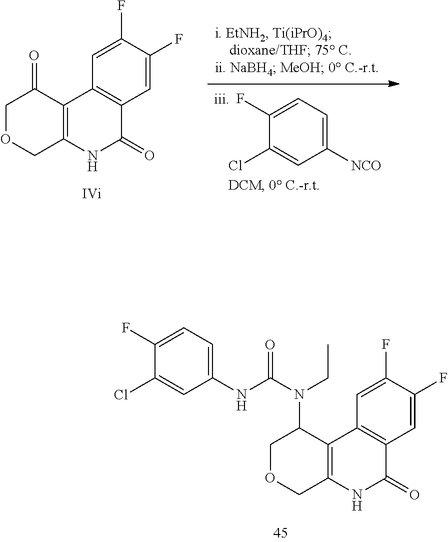

Racemic 3-(3-chloro-4-fluorophenyl)-1-(8,9-difluoro-6-oxo-1,4,5,6-tetrahydro-2H-pyrano[3,4-c]isoquinolin-1-yl)-1-ethylurea was synthesized in an analogous manner as described above for Compound 44, from 8,9-difluoro-4,5-dihydropyrano[3,4-c]isoquinoline-1,6-dione (IVi). LCMS: m/z found 452.1/454.2 [M+H]⁺; RT=4.46 min, (Method A); ¹H NMR (400 MHz, DMSO-d6) δ 11.67 (s, 1H), 8.50 (s, 1H), 8.11 (dd, 1H), 7.84 (dd, 1H), 7.53 (ddd, 1H), 7.44 (dd, 1H), 7.34 (t, 1H), 5.42 (d, 1H), 4.60 (d, 1H), 4.44 (d, 1H), 4.04 (d, 1H), 3.92 (dd, 1H), 3.43 (dq 1H), 3.28 (dd, 1H), 0.85 (t, 3H).

### (S)-1-(Ethyl((R)-1-(4-methoxyphenyl)ethyl)amino)-8,9-difluoro-1,5-dihydro-2H-pyrano[3,4-c]isoquinolin-6(4H)-one (XId)

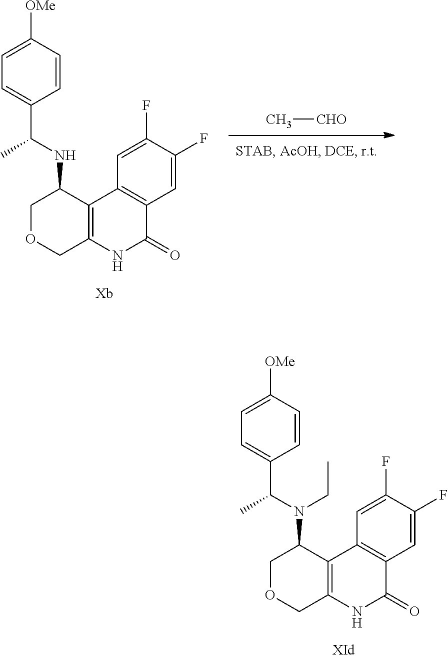

(S)-1-(Ethyl((R)-1-(4-methoxyphenyl)ethyl)amino)-8,9-difluoro-1,5-dihydro-2H-pyrano[3,4-c]isoquinolin-6(4H)-one was synthesized in an analogous manner as described above for XIa, in 79% yield, starting from from (S)-8,9-difluoro-1-(((R)-1-(4-methoxyphenyl)ethyl) amino)-1,5-dihydro-2H-pyrano[3,4-c]isoquinolin-6(4H)-one (Xb) and acetaldehyde. LCMS m/z found 415.4 [M+H]⁺; RT=0.67 min (Method B); ¹H NMR (400 MHz, CDCl₃) δ 12.10 (s, 1H), 8.11 (dd, 1H), 7.88 (dd, 1H), 7.08 (d, 2H), 6.79-6.70 (m, 2H), 4.74 (d, 1H), 4.61-4.48 (m, 2H), 4.17-4.05 (m, 2H), 3.75 (s, 3H), 3.66 (dd, 1H), 2.84 (dq, 1H), 2.72 (dq, 1H), 1.48 (d, 3H), 0.90 (t, 3H).

### (S)-3-(3-Chloro-4-fluorophenyl)-1-(8,9-difluoro-6-oxo-1,4,5,6-tetrahydro-2H-pyrano[3,4-c]isoquinolin-1-yl)-1-ethylurea (Compound 88)

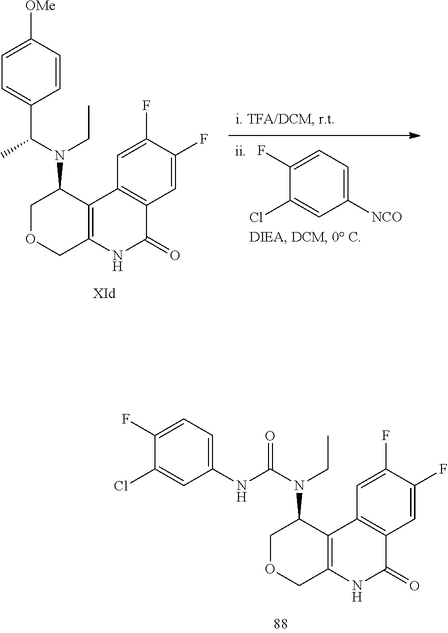

Optically pure (S)-3-(3-chloro-4-fluorophenyl)-1-(8,9-difluoro-6-oxo-1,4,5,6-tetrahydro-2H-pyrano[3,4-c]isoquinolin-1-yl)-1-ethylurea was synthesized in an analogous manner as described above for Compound 41, from (S)-1-(ethyl((R)-1-(4-methoxyphenyl)ethyl)amino)-8,9-difluoro-1,5-dihydro-2H-pyrano[3,4-c]isoquinolin-6(4H)-one (XId). LCMS m/z 452.2/454.3 [M+H]⁺; RT=4.49 min (Method A); ¹H NMR (400 MHz, DMSO-d₆) δ 11.67 (s, 1H), 8.50 (s, 1H), 8.11 (dd, 1H), 7.84 (ddd, 1H), 7.53 (dddd, 1H), 7.44 (dd, 1H), 7.34 (td, 1H), 5.42 (s, 1H), 4.60 (d, 1H), 4.44 (d, 1H), 4.04 (d, 1H), 3.92 (dd, 1H), 3.50-3.36 (m, 1H), 3.28 (dd, 1H), 0.85 (t, 3H); Chiral analytical SFC: RT=6.32 min, Column: OD-10-analytical; 25% Methanol; Total flow: 3 g/min; ee=99.99%.

### (S)-3-(3-Cyano-4-fluorophenyl)-1-(8,9-difluoro-6-oxo-1,4,5,6-tetrahydro-2H-pyrano[3,4-c]isoquinolin-1-yl)-1-ethylurea (Compound 138)

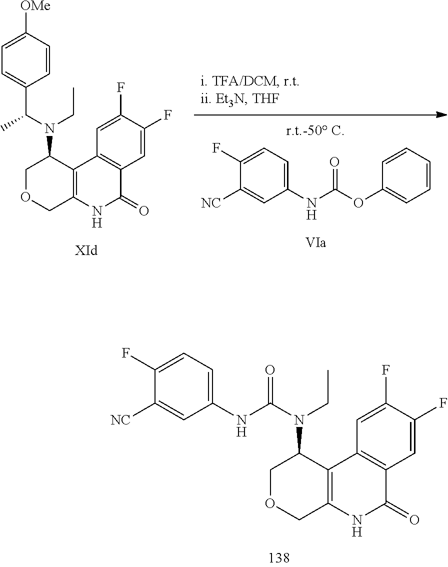 [SMILES: CCN([C@H](C)c1ccc(OC)cc1)[C@@H]1COCc2[nH]c(=O)c3cc(F)c(F)cc3c21 ; N#Cc1cc(NC(=O)Oc2ccccc2)ccc1F ; CCN(C(=O)Nc1ccc(F)c(C#N)c1)[C@@H]1COCc2[nH]c(=O)c3cc(F)c(F)cc3c21]

Optically pure (S)-3-(3-cyano-4-fluorophenyl)-1-(8,9-difluoro-6-oxo-1,4,5,6-tetrahydro-2H-pyrano[3,4-c]isoquinolin-1-yl)-1-ethylurea was synthesized in an analogous manner as above and as described for Compound 70, from (S)-1-(ethyl((R)-1-(4-methoxyphenyl)ethyl)amino)-8,9-difluoro-1,5-dihydro-2H-pyrano[3,4-c]isoquinolin-6(4H)-one (XId) and phenyl (3-cyano-4-fluorophenyl)carbamate (VIa). LCMS found m/z 443.15 [M+H]⁺, RT=2.88 min (Method C); ¹H NMR (400 MHz, DMSO-d₆) δ 11.68 (s, 1H), 8.66 (s, 1H), 8.11 (dd, 1H), 8.05 (dd, 1H), 7.91 (ddd, 1H), 7.53-7.37 (m, 2H), 5.42 (s, 1H), 4.60 (d, 1H), 4.49-4.40 (m, 1H), 4.09-3.99 (m, 1H), 3.92 (dd, 1H), 3.43 (dt, 1H), 3.34-3.23 (m, 1H), 0.86 (t, 3H).

### 8,9-Difluoro-1-(isobutylamino)-1,5-dihydro-2H-pyrano[3,4-c]isoquinolin-6(4H)-one (Vx)

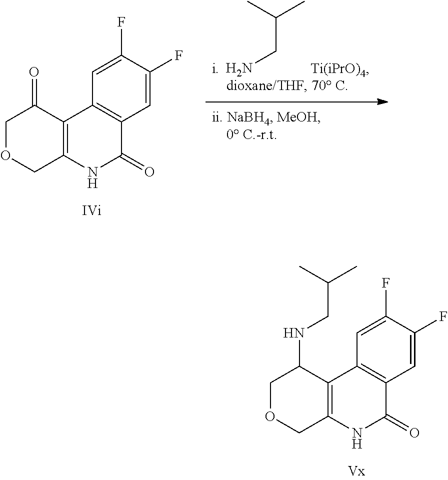

Racemic 8,9-difluoro-1-(isobutylamino)-1,5-dihydro-2H-pyrano[3,4-c]isoquinolin-6(4H)-one was synthesized in an analogous manner as described above for Vs, from 8,9-difluoro-4,5-dihydropyrano[3,4-c]isoquinoline-1,6-dione (IVi) and 2-methylpropan-1-amine. LCMS: m/z found 309.19 [M+H]⁺, RT=1.23 min (Method A).

### 3-(3-Cyano-4-fluorophenyl)-1-(8,9-difluoro-6-oxo-1,4,5,6-tetrahydro-2H-pyrano[3,4-c]isoquinolin-1-yl)-1-isobutylurea (Compounds 121 and 122)

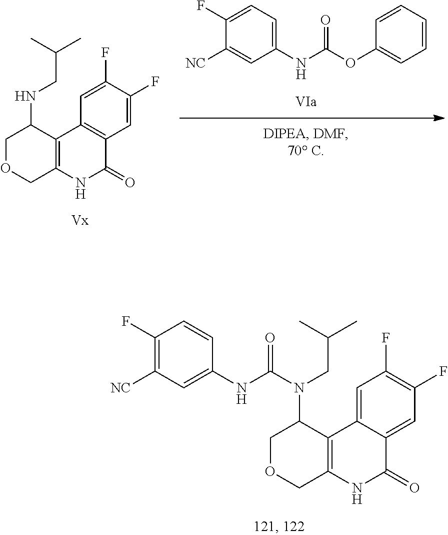 [SMILES: CC(C)CNC1COCc2[nH]c(=O)c3cc(F)c(F)cc3c21 ; N#Cc1cc(NC(=O)Oc2ccccc2)ccc1F ; CC(C)CN(C(=O)Nc1ccc(F)c(C#N)c1)C1COCc2[nH]c(=O)c3cc(F)c(F)cc3c21]

To a solution of 150 mg (0.48 mmol) of 8,9-difluoro-1-(isobutylamino)-1,5-dihydro-2H-pyrano[3,4-c]isoquinolin-6(4H)-one (Vx) in 4 mL of DMF at room temperature were added 0.26 mL (1.46 mmol) of DIPEA followed by 125 mg (0.48 mmol) of phenyl (3-cyano-4-fluorophenyl)carbamate (VIa) and the mixture was stirred at 70° C. for 3 hours. The reaction mixture was cooled to room temperature and diluted with ice-cold water (30 mL). The resulting precipitated was collected by filtration, washed with water (10 mL), n-pentane (10 mL) and dried under vacuum to afford 190 mg (0.40 mmol, 84%) of 3-(3-cyano-4-fluorophenyl)-1-(8,9-difluoro-6-oxo-1,4,5,6-tetrahydro-2H-pyrano[3,4-c]isoquinolin-1-yl)-1-isobutylurea. The enantiomers were subsequently separated by preparative SFC: Method isocratic, Mobile phase MeOH: CO₂—15:85, Column: Chiralpak IC (30×250 mm), 5μ, flow rate: 90 g/min.

Enantiomer I (Compound 121): LCMS: m/z found 471.3 [M+H]⁺, RT=4.41 min, (Method A); ¹H NMR (400 MHz, DMSO-d₆): δ 11.62 (br s, 1H), 8.74 (br s, 1H), 8.13-8.08 (m, 1H), 8.00-7.97 (m, 1H), 7.87-7.83 (m, 1H), 7.55-7.45 (m, 2H), 5.39 (s, 1H), 4.58 (d, 1H), 4.43 (d, 1H), 4.11 (d, 1H), 3.95-3.91 (m, 1H), 3.32-3.24 (m, 1H), 3.11-3.04 (m, 1H), 1.64-1.58 (m, 1H), 0.68 (d, 3H), 0.57 (d, 3H); Chiral analytical SFC RT=3.12 min, Column: Chiralpak IC (4.6×150 mm) 3μ, 20% Methanol, Flow rate: 3.0 g/min.

Enantiomer II (Compound 122): LCMS: m/z found 471.3 [M+H]⁺, RT=4.42 min, (Method A); ¹H NMR (400 MHz, DMSO-d₆): δ 11.62 (br s, 1H), 8.74 (br s, 1H), 8.13-8.08 (m, 1H), 8.00-7.97 (m, 1H), 7.87-7.83 (m, 1H), 7.55-7.45 (m, 2H), 5.39 (s, 1H), 4.58 (d, 1H), 4.43 (d, 1H), 4.11 (d, 1H), 3.95-3.91 (m, 1H), 3.32-3.24 (m, 1H), 3.11-3.04 (m, 1H), 1.64-1.58 (m, 1H), 0.68 (d, 3H), 0.57 (d, 3H); Chiral analytical SFC RT=3.90 min, Column: Chiralpak IC (4.6×150 mm) 3μ, 20% Methanol, Flow rate: 3.0 g/min.

### 2-((8,9-Difluoro-6-oxo-1,4,5,6-tetrahydro-2H-pyrano[3,4-c]isoquinolin-1-yl)amino)ethane-1-sulfonamide (Vsa)

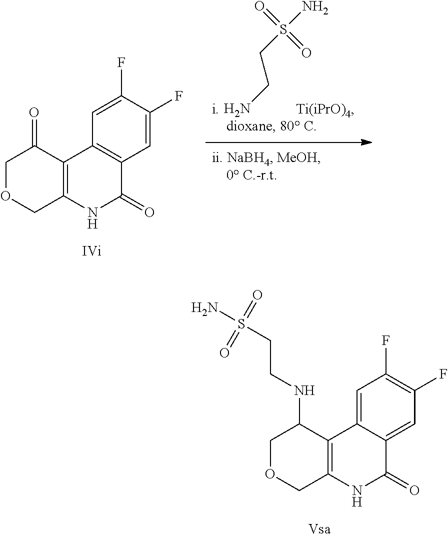 [SMILES: O=C1COCc2[nH]c(=O)c3cc(F)c(F)cc3c21 ; NCCS(N)(=O)=O ; NS(=O)(=O)CCNC1COCc2[nH]c(=O)c3cc(F)c(F)cc3c21]

2-((8,9-Difluoro-6-oxo-1,4,5,6-tetrahydro-2H-pyrano[3,4-c]isoquinolin-1-yl)amino)ethane-1-sulfonamide was synthesized in an analogous manner as described above, from 8,9-difluoro-4,5-dihydropyrano[3,4-c]isoquinoline-1,6-dione (IVi) and 2-aminoethane-1-sulfonamide. The crude product was purified by reverse-phase chromatography (REVELERIS® C-18-12 g column: 10-30% linear gradient of 0.1% formic acid in water with MeOH+THF (1:1)). LCMS: m/z found 360.13 [M+H]⁺, RT=1.05 min, (Method A); ¹H NMR (300 MHz, DMSO-d₆): δ 11.40 (br s, 1H), 8.07-8.00 (m, 1H), 7.85-7.78 (m, 1H), 6.76 (br s, 2H), 4.47-4.33 (m, 2H), 4.20 (d, 1H), 3.73 (s, 1H), 3.61-3.57 (m, 1H), 3.22-3.03 (m, 5H).

### 2-(3-(3-Chloro-4-fluorophenyl)-1-(8,9-difluoro-6-oxo-1,4,5,6-tetrahydro-2H-pyrano[3,4-c]isoquinolin-1-yl)ureido)ethane-1-sulfonamide (Compounds 136 and 137)

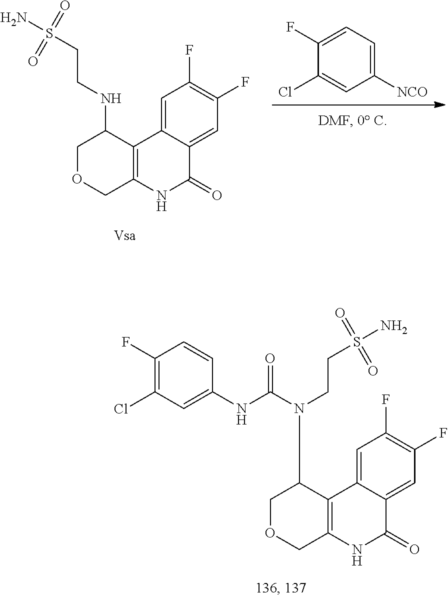

To a solution of 0.2 g (0.55 mmol) of 2-((8,9-difluoro-6-oxo-1,4,5,6-tetrahydro-2H-pyrano[3,4-c]isoquinolin-1-yl)amino)ethane-1-sulfonamide (Vsa) in 5 mL of DMF was added 95 mg (0.55 mmol) of 2-chloro-1-fluoro-4-isocyanatobenzene at 0° C. and the mixture was stirred for 1 hours. The mixture was diluted with water (20 mL), and the resulting solid was collected by filtration, washed with ethanol (5 mL) and dried under vacuum to afford 110 mg (0.20 mmol, 37%) of 2-(3-(3-chloro-4-fluorophenyl)-1-(8,9-difluoro-6-oxo-1,4,5,6-tetrahydro-2H-pyrano[3,4-c]isoquinolin-1-yl)ureido)ethane-1-sulfonamide. The enantiomers were subsequently separated by preparative SFC: Method isocratic, Mobile phase MeOH: CO₂—45:55, Column: Chiralpak IC (30×250 mm), 5μ, flow rate: 60 g/min.

Enantiomer I (Compound 136): LCMS: m/z found 531.2/533.2 [M+H]⁺, RT=3.99 min, (Method A); ¹H NMR (400 MHz, DMSO-d₆): δ 11.69 (br s, 1H), 8.76 (br s, 1H), 8.16-8.11 (m, 1H), 7.78-7.75 (m, 1H), 7.49-7.44 (m, 1H), 7.39-7.30 (m, 2H), 6.84 (br s, 2H), 5.30 (s, 1H), 4.62 (d, 1H), 4.45 (d, 1H), 4.12 (d, 1H), 3.92-3.88 (m, 1H), 3.77-3.69 (m, 1H), 3.45-3.38 (m, 1H), 3.32-3.26 (m, 1H), 3.08-3.03 (m, 1H); Chiral analytical SFC: RT=1.45 min, Column: Chiralpak IC-3 (4.6×150 mm) 3μ, 40% Methanol, Flow rate: 3.0 g/min.

Enantiomer II (Compound 137): LCMS: m/z found 531.2/533.2 [M+H]⁺, RT=3.99 min, (Method A); ¹H NMR (400 MHz, DMSO-d₆): δ 11.69 (br s, 1H), 8.76 (br s, 1H), 8.16-8.11 (m, 1H), 7.78-7.75 (m, 1H), 7.49-7.44 (m, 1H), 7.39-7.30 (m, 2H), 6.84 (br s, 2H), 5.30 (s, 1H), 4.62 (d, 1H), 4.45 (d, 1H), 4.12 (d, 1H), 3.92-3.88 (m, 1H), 3.77-3.69 (m, 1H), 3.45-3.38 (m, 1H), 3.32-3.26 (m, 1H), 3.08-3.03 (m, 1H); Chiral analytical SFC: RT=2.90 min, Column: Chiralpak IC-3 (4.6×150 mm) 3μ, 40% Methanol, Flow rate: 3.0 g/min.

### 8,9-Difluoro-1-((2-(methylsulfonyl)ethyl)amino)-1,5-dihydro-2H-pyrano[3,4-c]isoquinolin-6(4H)-one (Vsb)

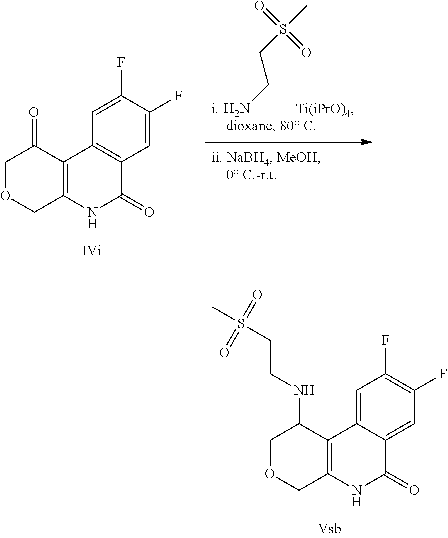

8,9-Difluoro-1-((2-(methylsulfonyl)ethyl)amino)-1,5-dihydro-2H-pyrano[3,4-c]isoquinolin-6(4H)-one was synthesized in an analogous manner as described above, from 8,9-difluoro-4,5-dihydropyrano[3,4-c]isoquinoline-1,6-dione (IVi) and 2-(methylsulfonyl)ethan-1-amine. LCMS: m/z found 359.17 [M+H]⁺, RT=1.46, (Method A); H NMR (400 MHz, CDCl₃): δ 10.01 (br s, 1H), 7.29-7.23 (m, 1H), 7.18-7.12 (m, 1H), 2.84-2.79 (m, 2H), 2.61-2.56 (m, 2H), 2.34 (s, 3H), 2.19-2.14 (m, 1H), 2.05-1.94 (m, 5H).

### 3-(3-Chloro-4-fluorophenyl)-1-(8,9-difluoro-6-oxo-1,4,5,6-tetrahydro-2H-pyrano[3,4-c]isoquinolin-1-yl)-1-(2-(methylsulfonyl)ethyl)urea (Compounds 139 and 140)

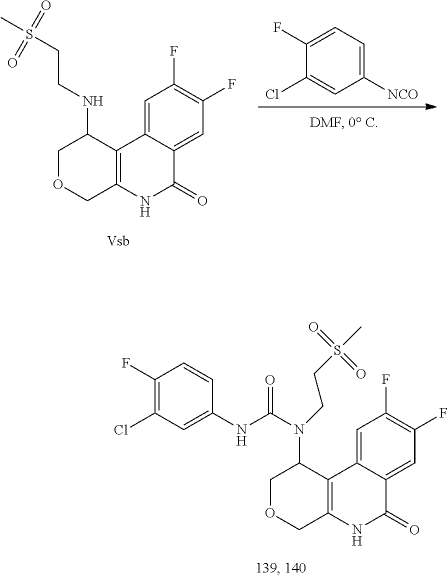

3-(3-Chloro-4-fluorophenyl)-1-(8,9-difluoro-6-oxo-1,4,5,6-tetrahydro-2H-pyrano[3,4-c]isoquinolin-1-yl)-1-(2-(methylsulfonyl)ethyl)urea was synthesized in an analogous manner as described above, from 8,9-difluoro-1-((2-(methylsulfonyl)ethyl)amino)-1,5-dihydro-2H-pyrano[3,4-c]isoquinolin-6(4H)-one (Vsb) and 2-chloro-1-fluoro-4-isocyanatobenzene. The enantiomers were subsequently separated by preparative SFC: Method isocratic, Mobile phase MeOH: CO₂—35:65, Column: Chiralcel OD-H (30×250 mm), 5μ, flow rate: 60 g/min.

Enantiomer I (Compound 139): LCMS: m/z found 530.2/532.2 [M+H]⁺, RT=4.19 min, (Method A); ¹H NMR (400 MHz, DMSO-d₆): δ 11.68 (br s, 1H), 8.78 (br s, 1H), 8.14-8.09 (m, 1H), 7.79-7.77 (m, 1H), 7.49-7.45 (m, 1H), 7.39-7.31 (m, 2H), 5.34 (s, 1H), 4.58 (d, 1H), 4.46 (d, 1H), 4.13 (d, 1H), 3.93-3.89 (m, 1H), 3.83-3.76 (m, 1H), 3.47-3.36 (m, 2H), 3.17-3.11 (m, 1H), 2.88 (s, 3H); Chiral analytical SFC: RT=2.87 min, Column: Chiralcel OD-3 (4.6×150 mm), 3μ, 25% Methanol, Flow rate: 3.0 g/min.

Enantiomer II (Compound 140): LCMS: m/z found 530.2/532.2 [M+H]⁺, RT=4.19 min, (Method A); ¹H NMR (400 MHz, DMSO-d₆): δ 11.68 (br s, 1H), 8.78 (br s, 1H), 8.14-8.09 (m, 1H), 7.79-7.77 (m, 1H), 7.49-7.45 (m, 1H), 7.39-7.31 (m, 2H), 5.34 (s, 1H), 4.58 (d, 1H), 4.46 (d, 1H), 4.13 (d, 1H), 3.93-3.89 (m, 1H), 3.83-3.76 (m, 1H), 3.47-3.36 (m, 2H), 3.17-3.11 (m, 1H), 2.88 (s, 3H); Chiral analytical SFC: RT=4.52 min, Column: Chiralcel OD-3 (4.6×150 mm), 3μ, 25% Methanol, Flow rate: 3.0 g/min.

### 6-Chloro-8,9-difluoro-2H-pyrano[3,4-c]isoquinolin-1(4H)-one (VIIa)

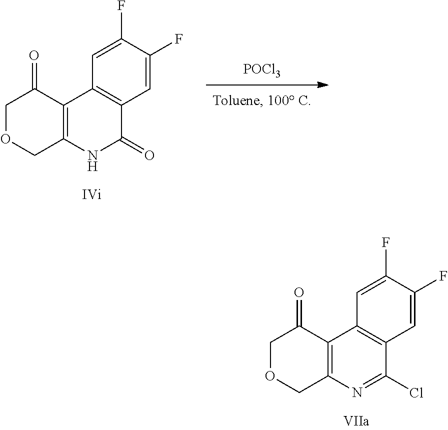 [SMILES: O=C1COCc2[nH]c(=O)c3cc(F)c(F)cc3c21 ; "POCl3" ; O=C1COCc2nc(Cl)c3cc(F)c(F)cc3c21]

A round bottom flask was charged with 3 g (11.9 mmol, 1 eq.) of 8,9-difluoro-2H-pyrano[3,4-c]isoquinoline-1,6(4H,5H)-dione (VIi) in 15 mL of toluene and 3.3 mL (35.8 mmol, 0.2 eq.) of POCl₃ were added under inert atmosphere. The reaction mixture was stirred for 4 h at 110° C. After completion of reaction, the mixture was basified with a saturated sodium bicarbonate solution (50 mL). The generated solid was filtered, and the filtrate was extracted with ethyl acetate (3×200 mL). The combined organic layers were washed with brine (100 mL), dried over anhydrous sodium sulfate, filtered and concentrated under reduced pressure to afford 1.6 g of 6-chloro-8,9-difluoro-2H-pyrano[3,4-c]isoquinolin-1(4H)-one (VIIa) as a pale yellow solid, which was taken into the step without purification. LCMS: m/z found 270.13 [M]⁻.

### 8,9-Difluoro-6-(methylamino)-2H1-pyrano[3,4-c]isoquinolin-1(4H)-one (VIIb)

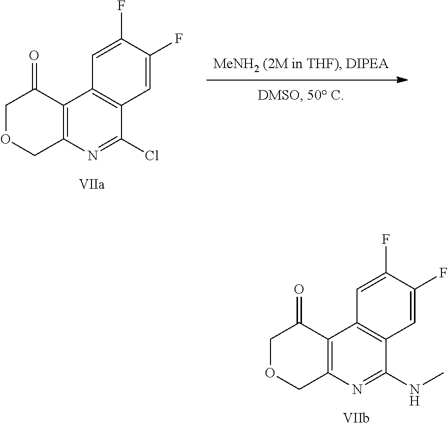 [SMILES: O=C1COCc2nc(Cl)c3cc(F)c(F)cc3c21 ; CNc1nc2c(c3cc(F)c(F)cc13)C(=O)COC2]

To a solution of 400 mg (1.48 mmol, 1 eq.) of 6-chloro-8,9-difluoro-2H-pyrano[3,4-c]isoquinolin-1(4H)-one (VIIa) in 4 mL of dimethyl sulfoxide in a sealed tube, 2.2 mL (4.4 mmol, 3 eq.) of methyl amine in THF(2M) and diisopropylethylamine (0.5 mL) were added and the reaction mixture was stirred at 50° C. for 16 h. After completion of reaction, the mixture was cooled to room temperature and poured into ice-cold water (20 mL), then extracted with ethyl acetate (2×50 mL). The combined organic layers were washed with brine (50 mL), dried over anhydrous sodium sulfate, filtered, and concentrated under reduced pressure. The resulting crude product was triturated with diethyl ether to afford 320 mg (81% yield) of 8,9-difluoro-6-(methylamino)-2H-pyrano[3,4-c]isoquinolin-1(4H)-one (VIIb) as a brown solid. LCMS: m/z found 265.34 [M]⁻.

### Benzyl(2-((8,9-difluoro-1-oxo-1,4-dihydro-2H1-pyrano[3,4-c]isoquinolin-6-yl)amino)ethyl)carbamate (VIIc)

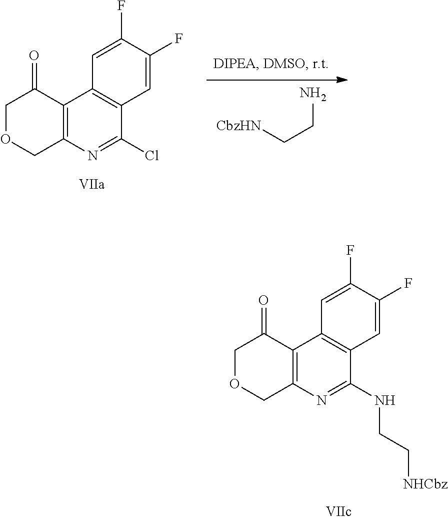 [SMILES: O=C1COCc2nc(Cl)c3cc(F)c(F)cc3c21 ; NCCNC(=O)OCc1ccccc1 ; O=C(NCCNc1nc2c(c3cc(F)c(F)cc13)C(=O)COC2)OCc1ccccc1]

To a solution of 600 mg (2.22 mmol, 1 eq.) of 6-chloro-8,9-difluoro-2H-pyrano[3,4-c]isoquinolin-1(4H)-one (VIIa) in 4 mL of DMSO in a sealed tube, were added 469 mg (2.6 mmol, 1.2 eq.) of benzyl (2-aminoethyl)carbamate and 0.77 mL (4.45 mmol, 3.0 eq.) of diisopropylethylamine, and mixture was stirred at room temperature for 16 h. After completion of reaction, the mixture was poured into ice-cold water (20 mL), then extracted with ethyl acetate (2×50 mL). The combined organic layers were washed with brine (50 mL), dried over anhydrous sodium sulfate, filtered, and concentrated under reduced pressure. The resulting product was triturated with diethyl ether to afford 650 mg (68% yield) of benzyl(2-((8,9-difluoro-1-oxo-1,4-dihydro-2H-pyrano[3,4-c]isoquinolin-6-yl)amino)ethyl)carbamate (VIIc) as a brown solid. LCMS: m/z found 265.34 [M]⁻.

### 2-((8,9-Difluoro-1-oxo-1,4-dihydro-2H1-pyrano[3,4-c]isoquinolin-6-yl)amino)ethyl acetate (VIId)

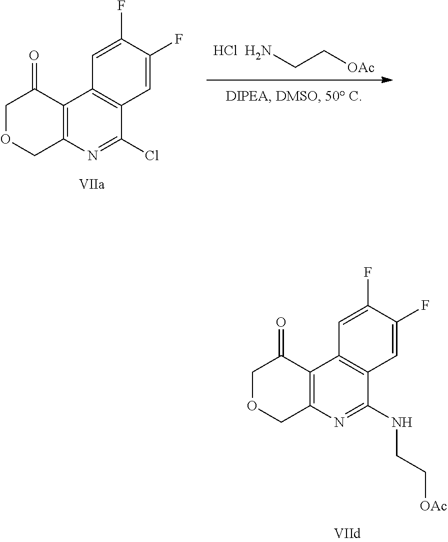 [SMILES: O=C1COCc2nc(Cl)c3cc(F)c(F)cc3c21 ; Cl ; CC(=O)OCCN ; CC(=O)OCCNc1nc2c(c3cc(F)c(F)cc13)C(=O)COC2]

To a solution of 500 mg (1.85 mmol, 1.0 eq) 6-chloro-8,9-difluoro-2H-pyrano[3,4-c]isoquinolin-1(4H)-one (VIIa) in 5 mL of DMSO in a sealed tube were added 309 mg (2.22 mmol, 1.2 eq) of 2-aminoethyl acetate hydrochloride and DIPEA (0.09 mL, 3.0 eq). The tube was capped and heated at 50° C. for 16 h. Upon cooling, the mixture was poured into ice cold water (20 mL) and extracted with EtOAC (2×50 mL). The combined organics were washed with brine (50 mL), dried over sodium sulfate, filtered, and concentrated under reduced pressure. The resulting crude product was triturated with diethyl ether to afford 420 mg (1.2 mmol, 67%) 2-((8,9-difluoro-1-oxo-1,4-dihydro-2H-pyrano[3,4-c]isoquinolin-6-yl)amino)ethyl acetate as a brown solid. LCMS: m/z found 335.17 [M+H]⁺; RT=1.83 min, (Method D).

### 8,9-Difluoro-N¹,N⁶-dimethyl-1,4-dihydro-2H1-pyrano[3,4-c]isoquinoline-1,6-diamine (V-Ba)

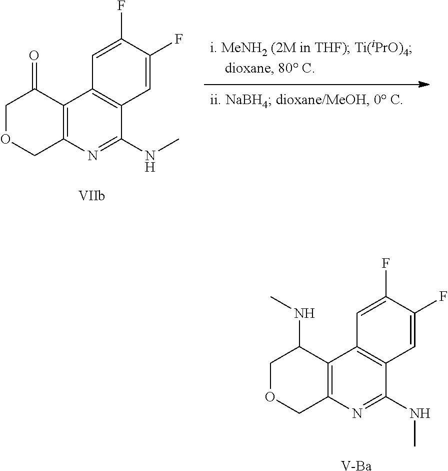 [SMILES: CNc1nc2c(c3cc(F)c(F)cc13)C(=O)COC2 ; CNc1nc2c(c3cc(F)c(F)cc13)C(NC)COC2 ; "V-Ba"]

To a stirred solution of 239 mg (0.9 mmol, 1 eq.) of 8,9-difluoro-6-(methylamino)-2H-pyrano[3,4-c]isoquinolin-1(4H)-one (VIIb) in 2.5 mL of THF at room temperature under inert atmosphere, 0.1 mL (2 mmol, 2.2 eq.) of a 2M methylamine solution in THF, followed by 0.72 mL of titanium isopropoxide were added and the mixture was stirred at 80° C. for 24 h. After imine formation, the reaction was cooled to 0° C. and diluted with anhydrous methanol (2 mL). To this mixture, 85 mg (2.2 mmol, 2.5 eq.) of NaBH₄ was added portionwise at 0° C. and the reaction mixture was stirred at room temperature for 4 h. After completion of reaction, the mixture was diluted with water (50 mL), filtered through Celite, and the filter cake was washed with ethyl acetate (50 ml). The organic layer was separated, and the aqueous layer was extracted with ethyl acetate (3×100 mL). The combined organic layers were dried over anhydrous sodium sulfate, filtered, and concentrated under reduced pressure. The resulting crude product was triturated with diethyl ether (10 mL), the resulting solids were collected by filtration, and dried under vacuum to afford 200 mg of racemic 8,9-difluoro-N¹,N⁶-dimethyl-1,4-dihydro-2H-pyrano[3,4-c]isoquinoline-1,6-diamine(V-Ba) as a light brown solid, which was carried i to the next step. LCMS: m/z found 251.19 [M+H]⁺.

### Benzyl(2-((8,9-difluoro-1-(methylamino)-1,4-dihydro-2H-pyrano[3,4-c]isoquinolin-6-yl)amino)ethyl)carbamate (V-Bb)

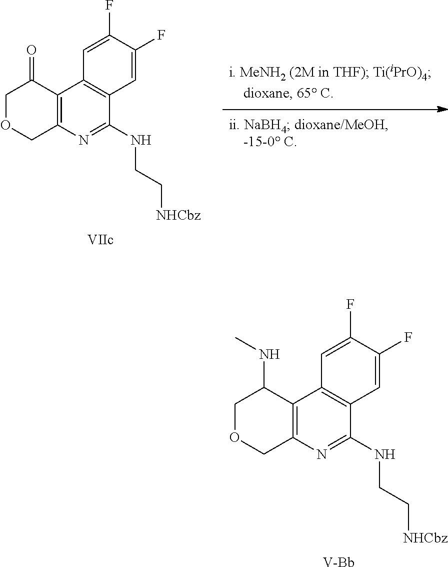 [SMILES: O=C(NCCNc1nc2c(c3cc(F)c(F)cc13)C(=O)COC2)OCc1ccccc1 ; "VIIc" ; CNC1COCc2nc(NCCNC(=O)OCc3ccccc3)c3cc(F)c(F)cc3c21 ; "V-Bb"]

Racemic benzyl(2-((8,9-difluoro-1-(methylamino)-1,4-dihydro-2H-pyrano[3,4-c]isoquinolin-6-yl)amino)ethyl)carbamate was synthesized in an analogous manner as described above, from benzyl(2-((8,9-difluoro-1-oxo-1,4-dihydro-2H-pyrano[3,4-c]isoquinolin-6-yl)amino)ethyl)carbamate (VIIc). LCMS: m/z found 443.29 [M+H]⁺.

### 2-((9-Fluoro-1-(methylamino)-1,4-dihydro-2H-pyrano[3,4-c]isoquinolin-6-yl)amino)ethan-1-ol (V-Bd)

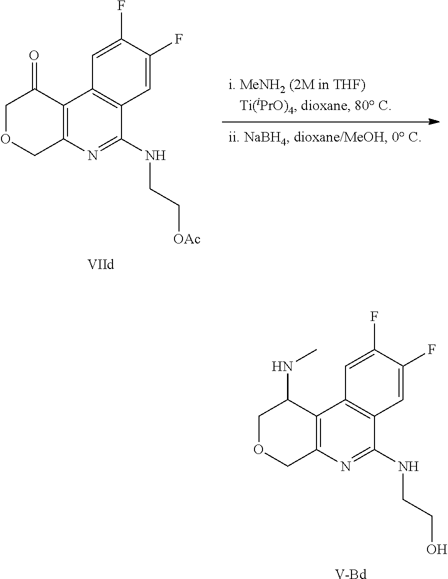 [SMILES: CC(=O)OCCNc1nc2c(c3cc(F)c(F)cc13)C(=O)COC2 ; "VIId" ; CNC1COCc2nc(NCCO)c3cc(F)c(F)cc3c21 ; "V-Bd"]

To a stirred solution of 240 mg (0.9 mmol, 1.0 eq) of 2-((8,9-difluoro-1-oxo-1,4-dihydro-2H-pyrano[3,4-c]isoquinolin-6-yl)amino)ethyl acetate(VIId) in 3 mL of THE at room temperature under inert atmosphere, was added 1.07 mL (1.96 mmol, 2.2 eq) of 2M methylamine solution in THE followed by 1.5 mL (5 vol) of titanium isopropoxide. The vessel was capped and heated at 50° C. for 24 h. The reaction was cooled to 0° C. and diluted with methanol (2 mL). To this mixture, 84 mg (2.2 mmol, 2.5 eq) of NaBH₄ was added portion-wise at 0° C. and stirred at room temperature for 4 h. The reaction mixture was diluted with water (50 mL), filtered and the filtrate washed with ethyl acetate (50 ml). The organic layer was separated, and the aqueous layer was extracted with ethyl acetate (3×100 mL). The combined organic layers were dried over anhydrous sodium sulfate and concentrated under reduced pressure. The resulting crude product was triturated with diethyl ether (10 mL). The resulting precipitate was collected and dried under vacuum to afford 250 mg of 2-((9-fluoro-1-(methylamino)-1,4-dihydro-2H-pyrano[3,4-c]isoquinolin-6-yl)amino)ethan-1-ol as a light brown solid, which was carried as such to the next step. LCMS: m/z found 310.26 [M+H]⁺; RT=0.86 min, (Method D).

### 1-(8,9-Difluoro-6-(methylamino)-1,4-dihydro-2H-pyrano[3,4-c]isoquinolin-1-yl)-3-(3-(difluoromethyl)-4-fluorophenyl)-1-methylurea (Compounds 235 and 236)

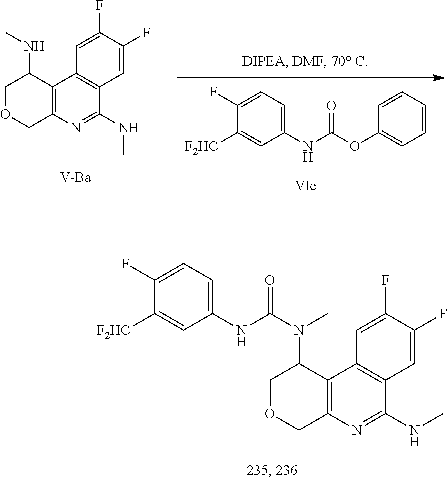 [SMILES: CNc1nc2c(c3cc(F)c(F)cc13)C(NC)COC2 ; O=C(Nc1ccc(F)c(C(F)F)c1)Oc1ccccc1 ; "V-Ba" ; CNc1nc2c(c3cc(F)c(F)cc13)C(N(C)C(=O)Nc1ccc(F)c(C(F)F)c1)COC2]

To a stirred solution of 200 mg (0.71 mmol, 1 eq.) of racemic 8,9-difluoro-N¹,N⁶-dimethyl-1,4-dihydro-2H-pyrano[3,4-c]isoquinoline-1,6-diamine (V-Ba) in 2 mL of DMF at room temperature were added 0.24 mL (0.86 mmol, 2 eq.) of diisopropylethylamine followed by 241 mg (0.86 mmol, 1 eq.) of phenyl (3-(difluoromethyl)-4-fluorophenyl)carbamate (VIe) under inert atmosphere. The reaction mixture was stirred at 70° C. for 1 h. After completion of reaction, the mixture was diluted with ice-cold water (40 mL). The precipitated solid was filtered, washed with water (10 mL), and dried under vacuum. The product was purified by MPLC (Grace system, Silica gel-40 g column; eluted with 5-10% linear gradient of methanol in dichloromethane) to afford 140 mg (42% yield) of racemic 1-(8,9-difluoro-6-(methylamino)-1,4-dihydro-2H-pyrano[3,4-c]isoquinolin-1-yl)-3-(3-(difluoromethyl)-4-fluorophenyl)-1-methylurea as an off white solid. LCMS: m/z found 467.30 [M+H]⁺. The enantiomers were subsequently separated by preparative SFC: Method isocratic, Mobile phase MeOH: CO₂—17:83, Column: Chiralpak IG (30×250 mm), 5μ, flow rate: 90 g/min.

Enantiomer I (Compound 235): LCMS: m/z found 467.1 [M+H]⁺, RT=3.31 min, (Method A); ¹H NMR (400 MHz, DMSO-d₆): δ 8.61 (br s, 1H), 8.35-8.30 (m, 1H), 7.88-7.87 (m, 1H), 7.74-7.66 (m, 2H), 7.55-7.50 (m, 1H), 7.34-7.07 (m, 2H), 5.54 (s, 1H), 4.68 (d, 1H), 4.55 (d, 1H), 4.11 (d, 1H), 3.96 (d, 1H), 2.95 (d, 3H), 2.75 (s, 3H); Chiral analytical SFC: RT=1.89 min, Column: Chiralpak IG-3 (4.6×150 mm), 3μ, 20% Methanol, Flow rate: 3.0 g/min.

Enantiomer II (Compound 236): LCMS: m/z found 467.1 [M+H]⁺, RT=3.31 min, (Method A); ¹H NMR (400 MHz, DMSO-d₆): δ 8.61 (br s, 1H), 8.35-8.30 (m, 1H), 7.88-7.87 (m, 1H), 7.74-7.66 (m, 2H), 7.55-7.50 (m, 1H), 7.34-7.07 (m, 2H), 5.54 (s, 1H), 4.68 (d, 1H), 4.55 (d, 1H), 4.11 (d, 1H), 3.96 (d, 1H), 2.95 (d, 3H), 2.75 (s, 3H); Chiral analytical SFC: RT=2.86 min, Column: Chiralpak IG-3 (4.6×150 mm), 3μ, 20% Methanol, Flow rate: 3.0 g/min.

### 3-(3-Chloro-4-fluorophenyl)-1-(8,9-difluoro-6-(methylamino)-1,4-dihydro-2H-pyrano[3,4-c]isoquinolin-1-yl)-1-methylurea (Compounds 241 and 242)

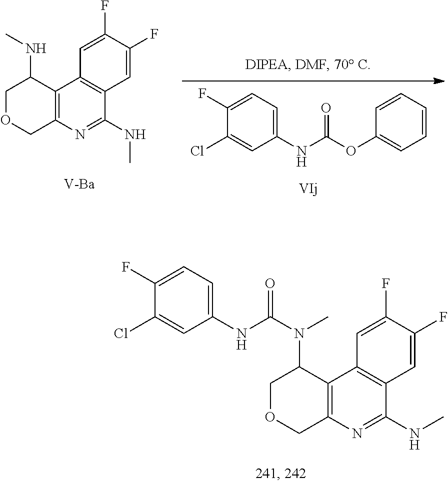 [SMILES: CNc1nc2c(c3cc(F)c(F)cc13)C(NC)COC2 ; O=C(Nc1ccc(F)c(Cl)c1)Oc1ccccc1 ; "V-Ba" ; CNc1nc2c(c3cc(F)c(F)cc13)C(N(C)C(=O)Nc1ccc(F)c(Cl)c1)COC2]

Racemic 3-(3-chloro-4-fluorophenyl)-1-(8,9-difluoro-6-(methylamino)-1,4-dihydro-2H-pyrano[3,4-c]isoquinolin-1-yl)-1-methylurea was synthesized in an analogous manner as described above, from 8,9-difluoro-N¹,N⁶-dimethyl-1,4-dihydro-2H-pyrano[3,4-c]isoquinoline-1,6-diamine (V-Ba) and phenyl (3-chloro-4-fluorophenyl)carbamate (VIj). The enantiomers were subsequently separated by preparative SFC: Method isocratic, Mobile phase MeOH: CO₂—20:80, Column: Chiralpak IG (30×250 mm), 5μ, flow rate: 100 g/min.

Enantiomer I (Compound 241): LCMS: m/z found 451.1 [M+H]⁺, RT=3.57 min, (Method A); ¹H NMR (400 MHz, DMSO-d₆): δ 8.56 (br s, 1H), 8.35-8.32 (m, 1H), 7.88-7.87 (m, 1H), 7.68 (d, 1H), 7.54-7.49 (m, 2H), 7.35-7.31 (t, 1H), 5.52 (s, 1H), 4.68 (d, 1H), 4.55 (d, 1H), 4.10 (d, 1H), 3.96 (d, 1H), 3.17 (d, 3H), 2.74 (s, 3H); Chiral analytical SFC: RT=3.0 min, Column: Chiralpak IG-3 (4.6×150 mm), 3μ, 20% Methanol, Flow rate: 3.0 g/min.

Enantiomer II (Compound 242): LCMS: m/z found 451.1 [M+H]⁺, RT=3.57 min, (Method A); ¹H NMR (400 MHz, DMSO-d₆): δ 8.56 (br s, 1H), 8.35-8.32 (m, 1H), 7.88-7.87 (m, 1H), 7.68 (d, 1H), 7.54-7.49 (m, 2H), 7.35-7.31 (t, 1H), 5.52 (s, 1H), 4.68 (d, 1H), 4.55 (d, 1H), 4.10 (d, 1H), 3.96 (d, 1H), 3.17 (d, 3H), 2.74 (s, 3H); Chiral analytical SFC: RT=5.74 min, Column: Chiralpak IG-3 (4.6×150 mm), 3μ, 20% Methanol, Flow rate: 3.0 g/min.

### 1-(6-((2-Aminoethyl)amino)-8,9-difluoro-1,4-dihydro-2H-pyrano[3,4-c]isoquinolin-1-yl)-3-(3-(difluoromethyl)-4-fluorophenyl)-1-methylurea (Compounds 239, 247, and 248)

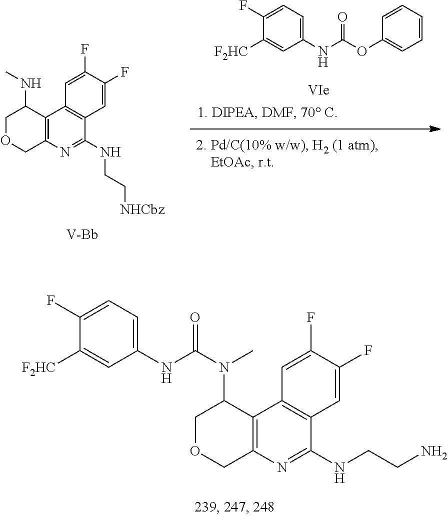 [SMILES: CNC1COCc2nc(NCCNC(=O)OCc3ccccc3)c3cc(F)c(F)cc3c21 ; O=C(Nc1ccc(F)c(C(F)F)c1)Oc1ccccc1 ; "V-Bb" ; CN(C(=O)Nc1ccc(F)c(C(F)F)c1)C1COCc2nc(NCCN)c3cc(F)c(F)cc3c21]

Step 1. Racemic benzyl (2-((1-(3-(3-(difluoromethyl)-4-fluorophenyl)-1-methylureido)-8,9-difluoro-1,4-dihydro-2H-pyrano[3,4-c]isoquinolin-6-yl)amino)ethyl)carbamate was synthesized in an analogous manner as described above, from benzyl(2-((8,9-difluoro-1-(methylamino)-1,4-dihydro-2H-pyrano[3,4-c]isoquinolin-6-yl)amino)ethyl)carbamate (V-Bb) and phenyl (3-(difluoromethyl)-4-fluorophenyl)carbamate (VIe). LCMS: m/z found 614.35.

Step 2. To a stirred solution of 200 mg (0.35 mmol, 1 eq.) of crude benzyl(2-((1-(5,6-difluoro-N-methyl-1H-indole-2-carboxamido)-8,9-difluoro-1,4-dihydro-2H-pyrano[3,4-c]isoquinolin-6-yl)amino)ethyl)carbamate, obtained as described above, in 10 mL of ethyl acetate was added 80 mg (0.195 mmol, 0.6 eq.) Pd/C. The reaction vessel was equipped with a hydrogen balloon and the reaction was continued for 16 h. After completion of the reaction, the mixture was filtered through a pad of celite, which was further washed with methanol and tetrahydrofuran (30 mL). Combined filtrates were concentrated under reduced pressure to afford 180 mg crude which was purified by achiral SFC to afford 80 mg (51% yield) of racemic 1-(6-((2-aminoethyl)amino)-8,9-difluoro-1,4-dihydro-2H-pyrano[3,4-c]isoquinolin-1-yl)-3-(3-(difluoromethyl)-4-fluorophenyl)-1-methylurea (Compound 239) as an off white solid. LCMS: m/z found 496.1 [M+H]⁺, RT=3.33 min, (Method A).

The enantiomers were subsequently separated by preparative SFC.

Enantiomer I (Compound 247): LCMS: m/z found 496.29 [M+H]⁺, RT=1.55 min, (Method D); ¹H NMR (400 MHz, DMSO-d₆): δ 8.61 (br s, 1H), 8.47-8.30 (m, 1H), 7.88-7.86 (m, 1H), 7.74-7.72 (m, 1H), 7.61-7.49 (m, 2H), 7.37-7.07 (m, 2H), 5.53 (s, 1H), 4.65 (d, 1H), 4.49 (d, 1H), 4.10 (d, 1H), 3.96 (d, 1H), 3.48-3.45 (m, 2H), 2.82-2.79 (m, 2H), 2.75 (s, 3H); Chiral analytical SFC: RT=4.1 min, Column: Lux Cellulose-2 (4.6×150 mm), 3μ, 30% (0.2% 7N Methanolic Ammonia in Acetonitrile:Methanol; 1:1), Flow rate: 3.0 g/min.

Enantiomer II (Compound 248): LCMS: m/z found 496.29 [M+H]⁺, RT=1.55 min, (Method D); ¹H NMR (400 MHz, DMSO-d₆): δ 8.61 (br s, 1H), 8.47-8.30 (m, 1H), 7.88-7.86 (m, 1H), 7.74-7.72 (m, 1H), 7.61-7.49 (m, 2H), 7.37-7.07 (m, 2H), 5.53 (s, 1H), 4.65 (d, 1H), 4.49 (d, 1H), 4.10 (d, 1H), 3.96 (d, 1H), 3.48-3.45 (m, 2H), 2.82-2.79 (m, 2H), 2.75 (s, 3H); Chiral analytical SFC: RT=6.58 min, Column: Lux Cellulose-2 (4.6×150 mm), 3μ, 30% (0.2% 7N Methanolic Ammonia in Acetonitrile:Methanol; 1:1), Flow rate: 3.0 g/min.

### N-(6-((2-Aminoethyl)amino)-8,9-difluoro-1,4-dihydro-2H-pyrano[3,4-c]isoquinolin-1-yl)-5,6-difluoro-N-methyl-1H-indole-2-carboxamide (Compounds 240, 249, and 250)

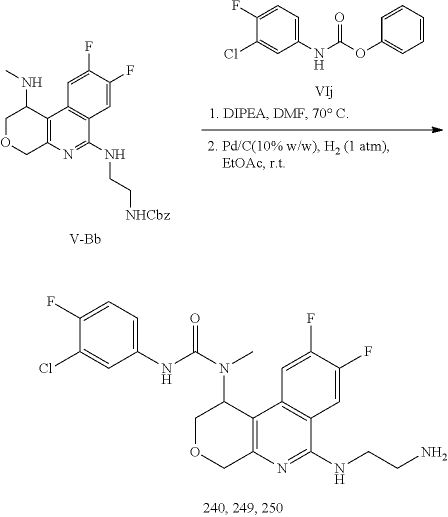 [SMILES: CNC1COCc2nc(NCCNC(=O)OCc3ccccc3)c3cc(F)c(F)cc3c21 ; O=C(Nc1ccc(F)c(Cl)c1)Oc1ccccc1 ; "V-Bb" ; CN(C(=O)Nc1ccc(F)c(Cl)c1)C1COCc2nc(NCCN)c3cc(F)c(F)cc3c21]

Racemic N-(6-((2-aminoethyl)amino)-8,9-difluoro-1,4-dihydro-2H-pyrano[3,4-c]isoquinolin-1-yl)-5,6-difluoro-N-methyl-1H-indole-2-carboxamide (Compound 240) was synthesized in an analogous manner as described above, from benzyl(2-((8,9-difluoro-1-(methylamino)-1,4-dihydro-2H-pyrano[3,4-c]isoquinolin-6-yl)amino)ethyl)carbamate (V-Bb) and phenyl (3-chloro-4-fluorophenyl)carbamate (VIj). LCMS: m/z found 480.1 [M+H]⁺, RT=2.35 min, (Method E); The enantiomers were subsequently separated by preparative SFC.

Enantiomer I (Compound 249): LCMS: m/z found 480.29/482.26 [M+H]⁺, RT=1.58 min, (Method D); ¹H NMR (400 MHz, DMSO-d₆): δ 8.57 (br s, 1H), 8.42-8.30 (m, 1H), 7.86-7.83 (m, 1H), 7.54-7.30 (m, 3H), 5.53 (s, 1H), 4.92 (bs, 1H), 4.61 (t, 1H), 4.52 (d, 1H), 4.08 (d, 1H), 3.95 (d, 1H), 3.57-3.54 (m, 2H), 3.17 (m, 2H), 2.76 (bs, 2H), 2.73 (s, 3H); Chiral analytical SFC: RT=2.14 min, Column: Chiralpak IC-3 (4.6×150 mm), 3μ, 30% (0.5% DEA in Methanol), Flow rate: 3.0 g/min.

Enantiomer II (Compound 250): LCMS: m/z found 480.29/482.26 [M+H]⁺, RT=1.58 min, (Method D); ¹H NMR (400 MHz, DMSO-d₆): δ 8.57 (br s, 1H), 8.42-8.30 (m, 1H), 7.86-7.83 (m, 1H), 7.54-7.30 (m, 3H), 5.53 (s, 1H), 4.92 (bs, 1H), 4.61 (t, 1H), 4.52 (d, 1H), 4.08 (d, 1H), 3.95 (d, 1H), 3.57-3.54 (m, 2H), 3.17 (m, 2H), 2.76 (bs, 2H), 2.73 (s, 3H); Chiral analytical SFC: RT=3.22 min, Column: Chiralpak IC-3 (4.6×150 mm), 3μ, 30% (0.5% DEA in Methanol), Flow rate: 3.0 g/min.

### 1-(8,9-Difluoro-6-((2-hydroxyethyl)amino)-1,4-dihydro-2H-pyrano[3,4-c]isoquinolin-1-yl)-3-(3-(difluoromethyl)-4-fluorophenyl)-1-methylurea (Compounds 237 and 238)

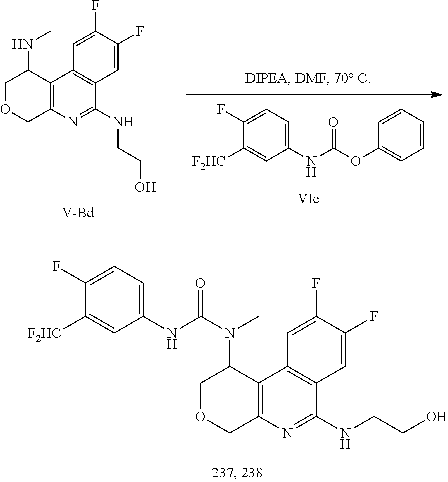 [SMILES: CNC1COCc2nc(NCCO)c3cc(F)c(F)cc3c21 ; O=C(Nc1ccc(F)c(C(F)F)c1)Oc1ccccc1 ; "V-Bd" ; CN(C(=O)Nc1ccc(F)c(C(F)F)c1)C1COCc2nc(NCCO)c3cc(F)c(F)cc3c21]

To a stirred solution of 200 mg (0.64 mmol, 1.0 eq) of 2-((8,9-difluoro-1-(methylamino)-1,4-dihydro-2H-pyrano[3,4-c]isoquinolin-6-yl)amino)ethan-1-ol (V-Bd) in 2 mL of DMF at room temperature was added 0.24 mL (0.86 mmol, 2.0 eq) of DIPEA followed by 145 mg (0.51 mmol, 0.8 eq) of phenyl (3-(difluoromethyl)-4-fluorophenyl)carbamate (1). The mixture was heated to 70° C. with stirring for 1 h. The reaction mixture was diluted with ice-cold water (40 mL). The precipitated solid was collected, washed with water (10 mL) and dried under vacuum. The obtained crude solid product was purified by MPLC (Grace system, Silica gel-40 g column; eluted with 5-10% linear gradient of methanol in dichloromethane) to afford 80 mg (0.16 mmol, 25%) of 1-(8,9-difluoro-6-((2-hydroxyethyl)amino)-1,4-dihydro-2H-pyrano[3,4-c]isoquinolin-1-yl)-3-(3-(difluoromethyl)-4-fluorophenyl)-1-methylurea. The enantiomers were subsequently separated by preparative SFC: Method isocratic, Mobile phase MeOH:CO₂—20:80. Column: Chiralpak-IG-3 (30×250 mm), 5μ, flow rate: 100 g/min.

Enantiomer I (Compound 237): LCMS m/z found 497.1 [M+H]⁺; RT=3.31 min, (Method A); ¹HNMR (400 MHz, DMSO-d6): δ 8.61 (s, 1H), 8.45-8.40 (m, 1H), 7.88-7.86 (m, 1H), 7.75-7.72 (m, 1H), 7.62-7.60 (m, 1H), 7.54-7.49 (m, 1H), 7.34-7.07 (m, 2H), 5.53 (s, 1H), 4.76 (t, 1H), 4.65 (d, 1H), 4.53 (d, 1H), 4.10 (d, 1H), 3.96 (m, 1H), 3.64-3.31 (m, 4H), 2.75 (s, 3H). Chiral analytical SFC: RT=2.08 min, Column: ChiralPak IG-3 (4.6×150 mm) 3 m, 20% Methanol, Flow rate: 3 g/min.

Enantiomer II (Compound 238): LCMS m/z found 497.1 [M+H]⁺; RT=3.29 min, (Method A); ¹HNMR (400 MHz, DMSO-d6): δ 8.61 (s, 1H), 8.45-8.40 (m, 1H), 7.88-7.86 (m, 1H), 7.75-7.72 (m, 1H), 7.62-7.60 (m, 1H), 7.54-7.49 (m, 1H), 7.34-7.07 (m, 2H), 5.53 (s, 1H), 4.76 (t, 1H), 4.65 (d, 1H), 4.53 (d, 1H), 4.10 (d, 1H), 3.96 (m, 1H), 3.64-3.31 (m, 4H), 2.75 (s, 3H). Chiral analytical SFC: RT=3.01 min, Column: ChiralPak IG-3 (4.6×150 mm) 3 m, 20% Methanol, Flow rate: 3 g/min.

### 3-(3-Chloro-4-fluorophenyl)-1-(8,9-difluoro-6-((2-hydroxyethyl)amino)-1,4-dihydro-2H-pyrano[3,4-c]isoquinolin-1-yl)-1-methylurea (Compounds 245 and 246)

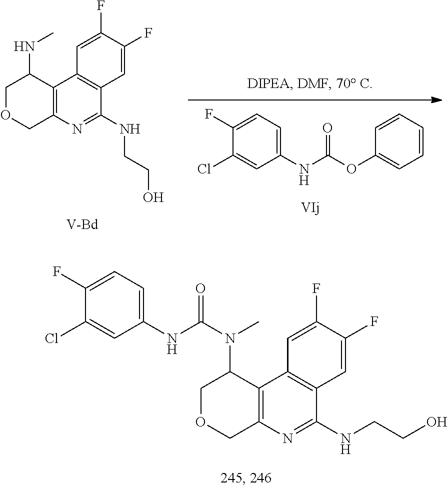 [SMILES: CNC1COCc2nc(NCCO)c3cc(F)c(F)cc3c21 ; O=C(Nc1ccc(F)c(Cl)c1)Oc1ccccc1 ; "V-Bd" ; CN(C(=O)Nc1ccc(F)c(Cl)c1)C1COCc2nc(NCCO)c3cc(F)c(F)cc3c21]

Racemic 3-(3-chloro-4-fluorophenyl)-1-(8,9-difluoro-6-((2-hydroxyethyl)amino)-1,4-dihydro-2H-pyrano[3,4-c]isoquinolin-1-yl)-1-methylurea was synthesized in an analogous manner as above from racemic 2-((8,9-difluoro-1-(methylamino)-1,4-dihydro-2H-pyrano[3,4-c]isoquinolin-6-yl)amino)ethan-1-ol (V-Bd) and phenyl (3-chloro-4-fluorophenyl)carbamate (VIj). The enantiomers were subsequently separated by preparative SFC: Method isocratic, Mobile phase MeOH:CO₂—25:75. Column: Chiralpak-IG-3 (30×250 mm), 5μ, flow rate: 110 g/min.

Enantiomer I (Compound 245): LCMS m/z found 481.0/483.0 [M+H]⁺; RT=3.48 min, (Method A); ¹HNMR (400 MHz, DMSO-d6): δ 8.56 (s, 1H), 8.45-8.40 (m, 1H), 7.86-7.83 (m, 1H), 7.63-7.60 (m, 1H), 7.54-7.48 (m, 2H), 7.36 (t, 1H), 5.52 (s, 1H), 4.76 (t, 1H), 4.65 (d, 1H), 4.53 (d, 1H), 4.10 (d, 1H), 3.96 (m, 1H), 3.64-3.31 (m, 4H), 2.74 (s, 3H). Chiral analytical SFC: RT=3.01 min, Column: ChiralPak IG-3 (4.6×150 mm) 3 μm, 20% Methanol, Flow rate: 3 g/min.

Enantiomer II (Compound 246): LCMS m/z found 481.0/483.1 [M+H]⁺; RT=3.48 min, (Method A); ¹HNMR (400 MHz, DMSO-d6): δ 8.56 (s, 1H), 8.45-8.40 (m, 1H), 7.86-7.83 (m, 1H), 7.63-7.60 (m, 1H), 7.54-7.48 (m, 2H), 7.36 (t, 1H), 5.52 (s, 1H), 4.76 (t, 1H), 4.65 (d, 1H), 4.53 (d, 1H), 4.10 (d, 1H), 3.96 (m, 1H), 3.64-3.31 (m, 4H), 2.74 (s, 3H). Chiral analytical SFC: RT=5.82 min, Column: ChiralPak IG-3 (4.6×150 mm) 3 μm, 20% Methanol, Flow rate: 3 g/min.

### 8,10-Difluoro-4,5-dihydropyrano[3,4-c]isoquinoline-1,6-dione (IVj)

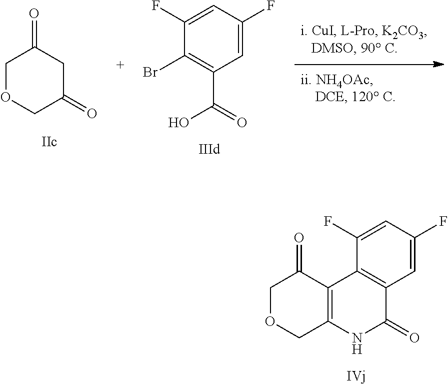 [SMILES: O=C1COCC(=O)C1 ; O=C(O)c1cc(F)cc(F)c1Br ; O=C1COCc2[nH]c(=O)c3cc(F)cc(F)c3c21]

8,10-Difluoro-4,5-dihydropyrano[3,4-c]isoquinoline-1,6-dione was synthesized in an analogous manner as described above, from tetrahydropyran-3,5-dione (IIc) and 2-bromo-3,5-difluoro-benzoic acid (IIId). LCMS: m/z found 252.1 [M+H]⁺; RT=0.87 min, (Method B); ¹H NMR (400 MHz, DMSO-d₆) δ 12.32 (s, 1H), 7.83-7.71 (m, 2H), 4.71 (s, 2H), 4.29 (s, 2H).

### 8,10-Difluoro-1-(methylamino)-1,2,4,5-tetrahydropyrano[3,4-c]isoquinolin-6-one (Vt)

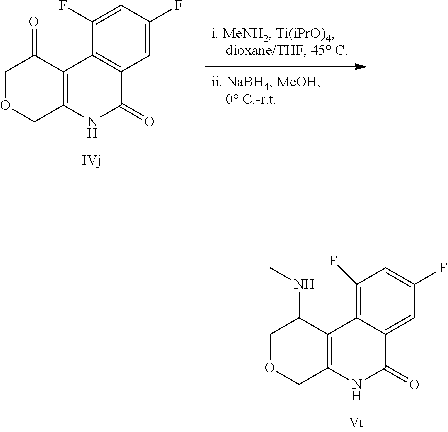 [SMILES: O=C1COCc2[nH]c(=O)c3cc(F)cc(F)c3c21 ; CNC1COCc2[nH]c(=O)c3cc(F)cc(F)c3c21]

8,10-Difluoro-1-(methylamino)-1,2,4,5-tetrahydropyrano[3,4-c]isoquinolin-6-one was synthesized in an analogous manner as described above, from 8,10-difluoro-4,5-dihydropyrano[3,4-c]isoquinoline-1,6-dione (IVj). LCMS m/z found 236.1 [M−MeNH]⁺; RT=0.70 min (Method B); ¹H NMR (400 MHz, CDCl₃) δ 7.98-7.89 (m, 1H), 7.25-7.14 (m, 1H), 4.69 (d, 1H), 4.59 (d, 1H), 4.34 (d, 1H), 3.88 (s, 1H), 3.65 (dd, 1H), 3.49 (s, 1H), 2.58 (s, 3H).

### 3-(3-Chloro-4-fluorophenyl)-1-(8,10-difluoro-6-oxo-1,4,5,6-tetrahydro-2H-pyrano[3,4-c]isoquinolin-1-yl)-1-methylurea (Compound 25)

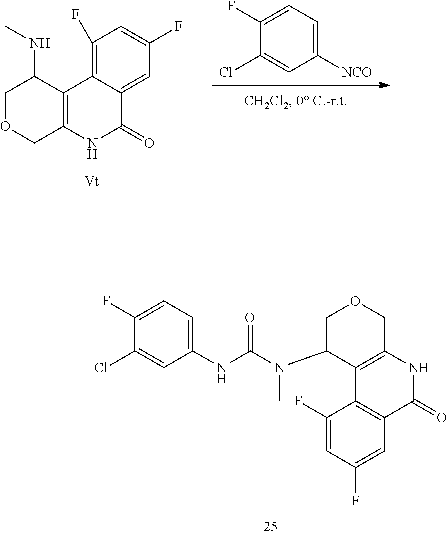

3-(3-Chloro-4-fluorophenyl)-1-(8,10-difluoro-6-oxo-1,4,5,6-tetrahydro-2H-pyrano[3,4-c]isoquinolin-1-yl)-1-methylurea was synthesized in an analogous manner as described above for 24, in 60% yield, from 8,10-difluoro-1-(methylamino)-1,2,4,5-tetrahydropyrano[3,4-c]isoquinolin-6-one (Vt). LCMS: m/z found 438.1/440.1 [M+H]⁺; RT=4.21 min (Method A); ¹H NMR (400 MHz, DMSO-d₆) δ 11.76 (s, 1H), 8.51 (s, 1H), 7.88-7.76 (m, 2H), 7.79-7.66 (m, 1H), 7.48 (ddd, 1H), 7.30 (t, 1H), 5.37 (s, 1H), 4.59 (d, 1H), 4.52-4.42 (m, 1H), 4.04 (dd, 1H), 3.85 (dd, 1H), 2.80 (s, 3H).

### 3-Methyl-2,3,4,5-tetrahydrophenanthridine-1,6-dione (IVk)

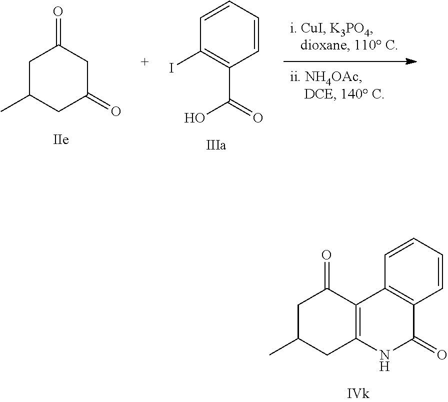

Racemic 3-methyl-2,3,4,5-tetrahydrophenanthridine-1,6-dione was synthesized in an analogous manner as described above for IVa, from 5-methylcyclohexane-1,3-dione (IIe) and 2-iodobenzoic acid (IIa). LCMS: m/z found 228.1 [M+H]⁺; RT=0.92 min (Method B); ¹H NMR (400 MHz, DMSO-d₆) δ 11.91 (s, 1H), 9.19 (dt, 1H), 8.19 (ddd, 1H), 7.75 (ddd, 1H), 7.49 (ddd, 1H), 2.87 (ddd, 1H), 2.70-2.60 (m, 1H), 2.64-2.45 (m, 1H), 2.39-2.23 (m, 2H), 1.05 (d, 3H).

### 3-Methyl-1-(methylamino)-2,3,4,5-tetrahydro-1H-phenanthridin-6-one (Vu, mixture of racemic cis/trans isomers)

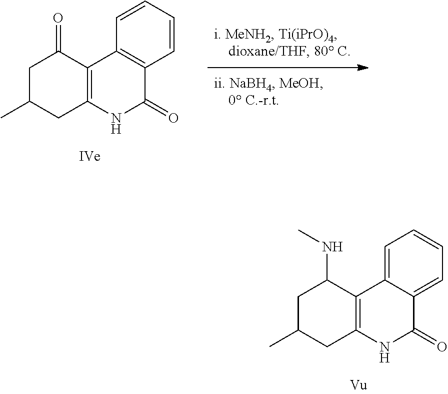 [SMILES: CC1CC(=O)c2c([nH]c(=O)c3ccccc23)C1 ; CNC1CC(C)Cc2[nH]c(=O)c3ccccc3c21]

3-Methyl-1-(methylamino)-2,3,4,5-tetrahydro-1H-phenanthridin-6-one (mixture of racemic cis/trans isomers) was synthesized in an analogous manner as described above, from racemic 3-methyl-2,3,4,5-tetrahydrophenanthridine-1,6-dione (IVk). ¹H NMR (400 MHz, CDCl₃) δ 11.59 (s, 1H), 8.63-8.29 (m, 1H), 7.84 (dd, 1H), 7.76-7.56 (m, 1H), 7.44 (ddd, 1H), 4.09 (t, 1H), 3.86 (dd, 1H)*, 2.85-2.73 (m, 1H)*, 2.67 (ddd, 1H), 2.59 (s, 1H), 2.51-2.35 (m, 5H), 2.35-2.09 (m, 1H), 1.89 (tdd, 1H), 1.40 (ddd, 1H), 1.35-1.23 (m, 1H)*, 1.15 (d, 3H) [“*” denotes distinguishable signals of the minor racemic diastereoisomer].

### 3-(3-Chloro-4-fluorophenyl)-1-methyl-1-(3-methyl-6-oxo-1,2,3,4,5,6-hexahydrophenanthridin-1-yl)urea (Compound 26, mixture of racemic cis/trans isomers)

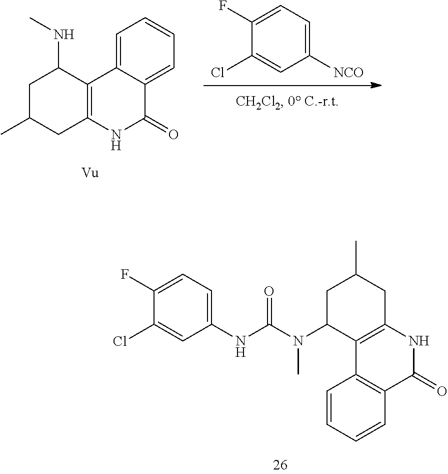

3-(3-Chloro-4-fluorophenyl)-1-methyl-1-(3-methyl-6-oxo-1,2,3,4,5,6-hexahydrophenanthridin-1-yl)urea was synthesized in an analogous manner as described above for 24, in 81% yield, as a mixture of 85% racemic cis and 15% racemic trans isomers, from 3-methyl-1-(methylamino)-2,3,4,5-tetrahydro-1H-phenanthridin-6-one (Vu). LCMS: m/z found 414.2/416.2 [M+H]⁺; RT=4.56 min (major isomer); m/z found 414.2/416.2 [M+H]⁺; RT=4.59 min (minor isomer) (Method A); ¹H NMR (400 MHz, DMSO-d₆) δ 11.33 (s, 1H)*, 11.28 (s, 1H), 8.19 (ddt, 1H), 7.91 (dd, 1H)*, 7.88 (dd, 1H), 7.74-7.69 (m, 1H)*, 7.69-7.62 (m, 1H), 7.53 (dtd, 1H), 7.49-7.40 (m, 2H), 7.38 (d, 1H)*, 7.32 (td, 1H), 5.75 (s, 1H), 5.57 (s, 1H)*, 2.75 (dd, 1H)*, 2.66 (s, 1H), 2.45 (s, 3H), 2.33 (dd, 1H), 2.25-2.16 (m, 1H)*, 2.12 (d, 1H), 1.94 (d, 1H)*, 1.82 (s, 1H), 1.59 (td, 1H)*, 1.33 (q, 1H), 1.03 (dd, 3H), 1.03 (dd, 3H, overlapped)* [“*” denotes distinguishable signals of the minor isomer].

### 3,3-Dimethyl-4,5-dihydro-2H-phenanthridine-1,6-dione (IVm)

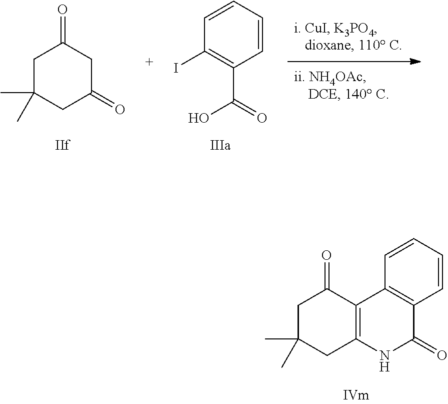

3,3-Dimethyl-4,5-dihydro-2H-phenanthridine-1,6-dione was synthesized in an analogous manner as described above for IVa, from 5,5-dimethylcyclohexane-1,3-dione (IIf) and 2-iodobenzoic acid (IIIa). LCMS: m/z found 242.1 [M+H]⁺; RT=0.97 min (Method B); ¹H NMR (400 MHz, DMSO-d₆) δ 11.91 (s, 1H), 9.20 (ddd, 1H), 8.20 (ddd, 1H), 7.76 (ddd, 1H), 7.50 (ddd, 1H), 2.79 (s, 2H), 2.45 (s, 2H), 1.06 (s, 6H).

### 3,3-Di ethyl-1-(methylamino)-1,2,4,5-tetrahydrophenanthridin-6-one (Vv)

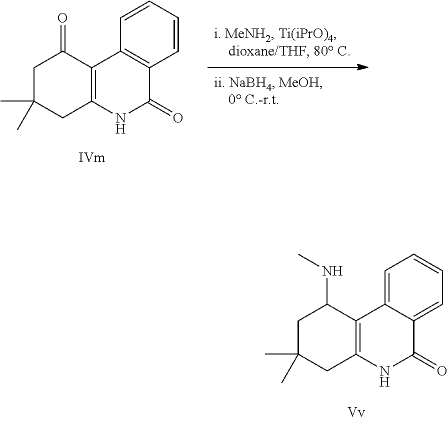 [SMILES: CC1(C)CC(=O)c2c([nH]c(=O)c3ccccc23)C1 ; CNC1CC(C)(C)Cc2[nH]c(=O)c3ccccc3c21]

3,3-Dimethyl-1-(methylamino)-2,3,4,5-tetrahydro-1H-phenanthridin-6-one was synthesized in an analogous manner as described above, from 3,3-dimethyl-4,5-dihydro-2H-phenanthridine-1,6-dione (IVm). ¹H NMR (400 MHz, CDCl₃) δ 11.01 (s, 1H), 8.45 (dd, 1H), 7.82 (dt, 1H), 7.70 (ddd, 1H), 7.45 (ddd, 1H), 3.95 (t, 1H), 2.62 (d, 1H), 2.50 (s, 3H), 2.43 (d, 1H), 1.99-1.89 (m, 1H), 1.74 (dd, 1H), 1.20 (s, 3H), 1.01 (s, 3H).

### 3-(3-Chloro-4-fluorophenyl)-1-(3,3-dimethyl-6-oxo-1,2,3,4,5,6-hexahydrophenanthridin-1-yl)-1-methylurea (Compound 27)

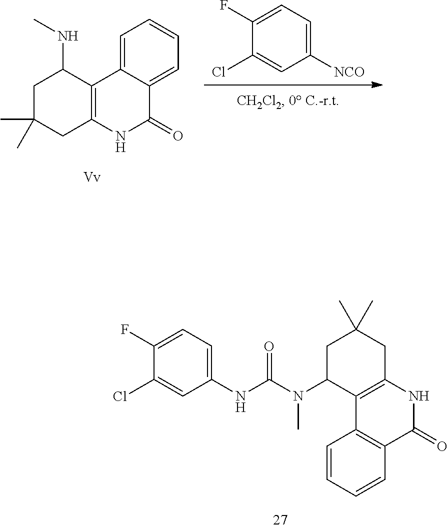

3-(3-Chloro-4-fluorophenyl)-1-(3,3-dimethyl-6-oxo-1,2,3,4,5,6-hexahydrophenanthridin-1-yl)-1-methylurea was synthesized in an analogous manner as described above for 24, from 3,3-dimethyl-1-(methylamino)-1,2,4,5-tetrahydrophenanthridin-6-one (Vv). LCMS: m/z found 428.2/430.2 [M+H]⁺; RT=4.74 min (Method A); ¹H NMR (400 MHz, DMSO-d₆) δ 11.27 (s, 1H), 8.44 (s, 1H), 8.24-8.16 (m, 1H), 7.89 (dd, 1H), 7.67 (ddd, 1H), 7.56-7.38 (m, 3H), 7.32 (t, 1H), 5.68 (s, 1H), 2.62 (d, 1H), 2.47 (s, 3H), 2.22 (dd, 1H), 1.84 (d, 1H), 1.51 (dd, 1H), 1.08 (s, 3H), 0.90 (s, 3H).

### 7,8-Difluoro-2,3,4,5-tetrahydrophenanthridine-1,6-dione (IVn)

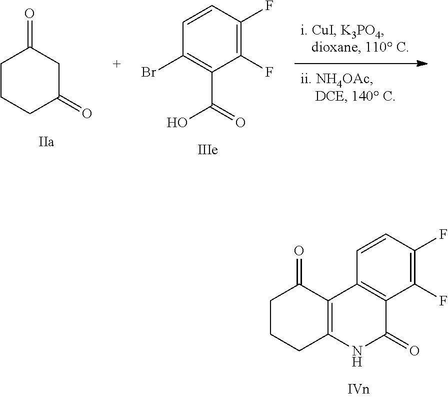 [SMILES: O=C1CCCC(=O)C1 ; O=C(O)c1c(Br)ccc(F)c1F ; O=C1CCCc2[nH]c(=O)c3c(F)c(F)ccc3c21]

7,8-difluoro-2,3,4,5-tetrahydrophenanthridine-1,6-dione was synthesized in an analogous manner as described for IVa, from cyclohexane-1,3-dione (IIa) and 2-bromo-3,4-difluoro-benzoic acid (IIe). LCMS: m/z found 250.1 [M+H]⁺; RT=0.87 min (Method B); ¹H NMR (400 MHz, DMSO-d₆) δ 12.05 (s, 1H), 8.05 (ddd, 1H), 7.59 (ddd, 1H), 2.80 (t, 2H), 2.53 (d, 2H), 2.07-1.96 (m, 2H).

### 7,8-Difluoro-1-(methylamino)-2,3,4,5-tetrahydro-1H-phenanthridin-6-one (Vw)

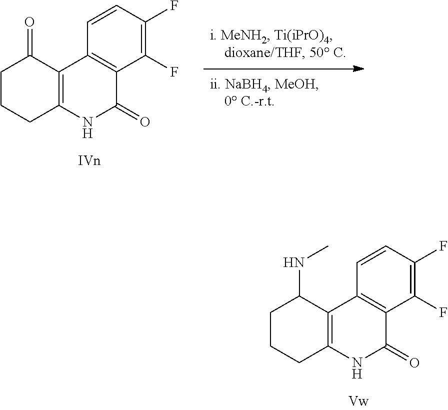 [SMILES: O=C1CCCc2[nH]c(=O)c3c(F)c(F)ccc3c21 ; CNC1CCCc2[nH]c(=O)c3c(F)c(F)ccc3c21]

7,8-Difluoro-1-(methylamino)-2,3,4,5-tetrahydro-1H-phenanthridin-6-one was synthesized in an analogous manner as described above, from 7,8-difluoro-2,3,4,5-tetrahydrophenanthridine-1,6-dione (IVn). LCMS: m/z found 265.28 [M+H]⁺; RT=0.71 min (Method B); ¹H NMR (400 MHz, CDCl₃) δ 8.22 (ddd, 1H), 7.23 (td, 1H), 4.17 (s, 1H), 2.74-2.61 (m, 2H), 2.55 (s, 3H), 2.25-2.16 (m, 1H), 2.07 (s, 1H), 1.78 (d, 1H), 1.56 (t, 1H).

### 3-(3-Chloro-4-fluorophenyl)-1-(7,8-difluoro-6-oxo-1,2,3,4,5,6-hexahydrophenanthridin-1-yl)-1-methylurea (Compound 28)

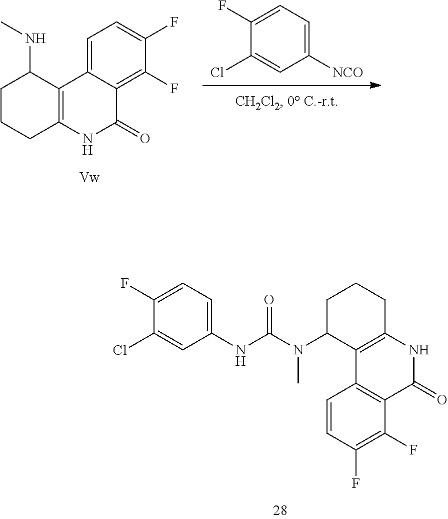

3-(3-Chloro-4-fluorophenyl)-1-(7,8-difluoro-6-oxo-1,2,3,4,5,6-hexahydrophenanthridin-1-yl)-1-methylurea was synthesized in an analogous manner as described above for 24, from 7,8-difluoro-1-(methylamino)-2,3,4,5-tetrahydro-1H-phenanthridin-6-one (Vw). LCMS: m/z found 436.1 [M+H]⁺; RT=4.36 min (Method A); ¹H NMR (400 MHz, DMSO-d₆) δ 11.56 (s, 1H), 8.39 (s, 1H), 8.11 (ddd, 1H), 7.86 (dd, 1H), 7.57-7.45 (m, 2H), 7.30 (t, 1H), 5.57 (s, 1H), 2.69 (s, 3H), 2.59 (dt, 1H), 2.02-1.94 (m, 1H), 1.91-1.59 (m, 4H).

### 7,8-Difluoro-4,5-dihydropyrano[3,4-c]isoquinoline-1,6-dione (IVo)

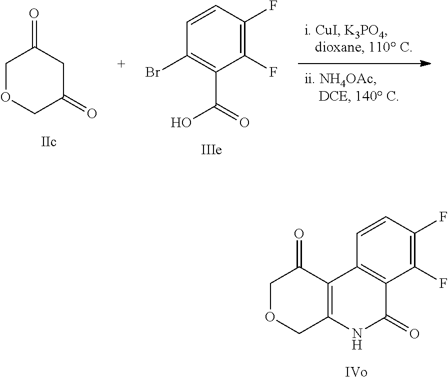 [SMILES: O=C1COCC(=O)C1 ; O=C(O)c1c(Br)ccc(F)c1F ; O=C1COCc2[nH]c(=O)c3c(F)c(F)ccc3c21]

7,8-Difluoro-4,5-dihydropyrano[3,4-c]isoquinoline-1,6-dione was synthesized in an analogous manner as described above, from tetrahydropyran-3,5-dione (IIc) and 2-bromo-3,4-difluoro-benzoic acid (IIIe). LCMS: m/z found 252.1 [M+H]⁺; RT=0.65 min (Method B); ¹H NMR (400 MHz, DMSO-d₆) δ 12.29 (s, 1H), 8.10 (ddd, 1H), 7.67 (ddd, 1H), 4.72 (s, 2H), 4.31 (s, 2H).

### 7,8-Difluoro-1-(methylamino)-1,2,4,5-tetrahydropyrano[3,4-c]isoquinolin-6-one (Vy)

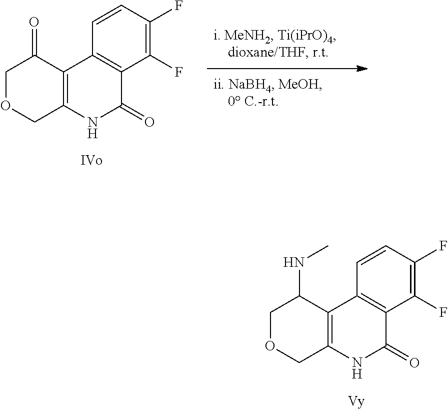 [SMILES: O=C1COCc2[nH]c(=O)c3c(F)c(F)ccc3c21 ; CNC1COCc2[nH]c(=O)c3c(F)c(F)ccc3c21]

7,8-Difluoro-1-(methylamino)-1,2,4,5-tetrahydropyrano[3,4-c]isoquinolin-6-one was synthesized in an analogous manner as described above, from 7,8-difluoro-4,5-dihydropyrano[3,4-c]isoquinoline-1,6-dione (IVo). LCMS: m/z found 267.2 [M+H]⁺; RT=0.46 min (Method B); ¹H NMR (400 MHz, CDCl₃) δ 8.23 (ddd, 1H), 7.28 (td, 1H), 4.71 (d, 1H), 4.65-4.55 (m, 1H), 4.35 (dd, 1H), 3.89 (s, 1H), 3.65 (dd, 1H), 2.58 (s, 3H).

### 3-(3-Chloro-4-fluorophenyl)-1-(7,8-difluoro-6-oxo-1,4,5,6-tetrahydro-2H-pyrano[3,4-c]isoquinolin-1-yl)-1-methylurea (Compound 35)

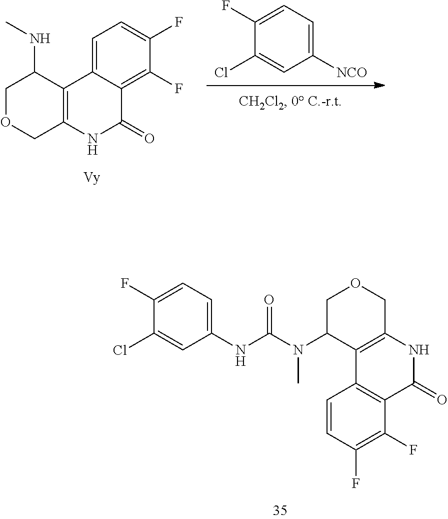

3-(3-Chloro-4-fluorophenyl)-1-(7,8-difluoro-6-oxo-1,4,5,6-tetrahydro-2H-pyrano[3,4-c]isoquinolin-1-yl)-1-methylurea was synthesized in an analogous manner as described above, from racemic 7,8-difluoro-1-(methylamino)-1,2,4,5-tetrahydropyrano[3,4-c]isoquinolin-6-one (Vy). LCMS: m/z found 438.1/440.2 [M+H]⁺; RT=3.88 min (Method A); ¹H NMR (400 MHz, DMSO-d₆) δ 11.69 (s, 1H), 8.52 (s, 1H), 8.13 (dd, 1H), 7.84 (dd, 1H), 7.56 (td, 1H), 7.49 (ddd, 1H), 7.30 (t, 1H), 5.38 (s, 1H), 4.60 (d, 1H), 4.48 (d, 1H), 4.06 (d, 1H), 3.86 (dd, 1H), 2.82 (s, 3H).

### Phenyl N-(3-chloro-5-fluoro-phenyl)carbamate (VIb)

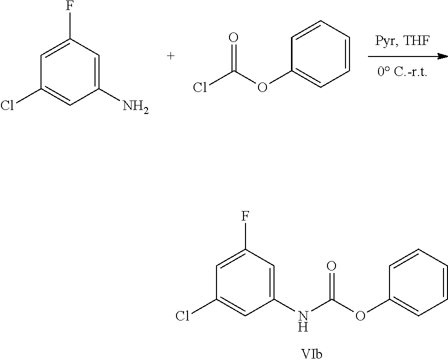 [SMILES: Nc1cc(F)cc(Cl)c1 ; O=C(Cl)Oc1ccccc1 ; O=C(Nc1cc(F)cc(Cl)c1)Oc1ccccc1]

A mixture of 3-chloro-5-fluoro-aniline (1.0 g, 6.87 mmol) and pyridine (2.2 mL, 27.48 mmol) in 10 mL of anhydrous THE was cooled to 0° C. under a nitrogen atmosphere. Phenyl chloroformate (0.95 mL, 7.56 mmol) was added slowly, the ice bath was removed and the mixture was stirred at room temperature for 16 hours. The reaction mixture was diluted with 30 mL of water, and extracted with EtOAc (2×35 mL). The combined organic extracts were washed with brine (10 mL), dried over sodium sulfate, filtered and evaporated to dryness. The product was isolated by flash chromatography (Silicagel, EtOAc/hexanes 0-20%) and dried under high vacuum to provide phenyl N-(3-chloro-5-fluoro-phenyl)carbamate (1.39 g, 76.2%) as a white solid. LCMS: m/z found 266.2 [M+H]⁺; RT=1.29 min (Method B); ¹H NMR (400 MHz, CDCl₃) δ 7.44-7.37 (m, 2H), 7.30-7.24 (m, 1H), 7.24-7.20 (m, 2H), 7.20-7.15 (m, 2H), 7.05 (s, 1H), 6.83 (ddd, 1H).

### 3-(3-Chloro-5-fluorophenyl)-1-methyl-1-(6-oxo-1,4,5,6-tetrahydro-2H-pyrano[3,4-c]isoquinolin-1-yl)urea (Compound 29)

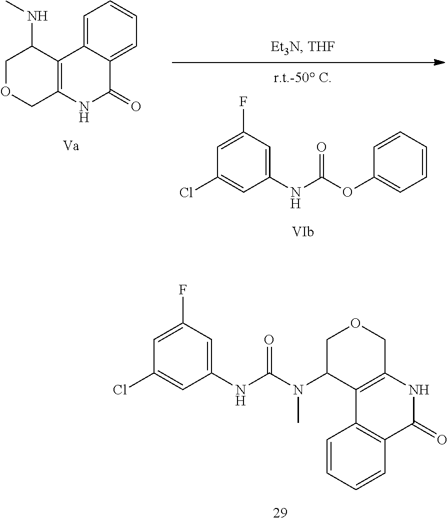 [SMILES: CNC1COCc2[nH]c(=O)c3ccccc3c21 ; O=C(Nc1cc(F)cc(Cl)c1)Oc1ccccc1 ; CN(C(=O)Nc1cc(F)cc(Cl)c1)C1COCc2[nH]c(=O)c3ccccc3c21]

Triethylamine (45 uL, 0.33 mmol) was added to 1-(methylamino)-1,2,4,5-tetrahydropyrano[3,4-c]isoquinolin-6-one (Va, 30 mg, 0.13 mmol) in 1.5 mL of anhydrous THF. A solution of phenyl N-(3-chloro-5-fluoro-phenyl)carbamate (VIb, 31.2 mg, 0.12 mmol) in 0.5 mL anhydrous THE was added, and the reaction mixture was stirred at room temperature for 45 min, then at 50° C. for 2 hours. The reaction mixture was diluted with 30 mL EtOAc and washed with 0.2 M HCl (10 mL), dil. NaHCO₃(15 mL), then with brine, and dried over sodium sulfate. The organic solution was filtered, and the solvent was evaporated to a white solid, which was triturated from methanol. The product was collected by filtration, washed with methanol, then with 1:1 v/v methanol/dichloromethane, then hexane, and dried overnight under high vacuum at 50° C., to provide 3-(3-chloro-5-fluorophenyl)-1-methyl-1-(6-oxo-1,4,5,6-tetrahydro-2H-pyrano[3,4-c]isoquinolin-1-yl)urea (28.1 mg, 54%). LCMS: m/z found 402.2/404.2 [M+H]⁺; RT=4.33 min (Method A); ¹H NMR (400 MHz, DMSO-d₆) δ 11.44 (s, 1H), 8.72 (s, 1H), 8.22 (ddd, 1H), 7.79-7.70 (m, 1H), 7.59 (td, 1H), 7.58-7.44 (m, 3H), 6.99 (ddd, 1H), 5.43 (s, 1H), 4.58 (d, 1H), 4.44 (dd, 1H), 4.13-4.02 (m, 1H), 3.94 (dd, 1H), 2.81 (s, 3H).

### 8-Chloro-4,5-dihydropyrano[3,4-c]isoquinoline-1,6-dione (IVp)

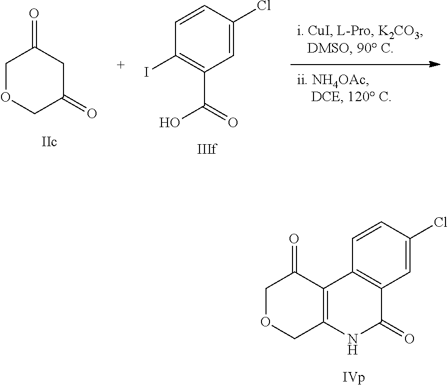 [SMILES: O=C1COCC(=O)C1 ; O=C(O)c1cc(Cl)ccc1I ; O=C1COCc2[nH]c(=O)c3cc(Cl)ccc3c21]

8-Chloro-4,5-dihydropyrano[3,4-c]isoquinoline-1,6-dione was synthesized in an analogous manner as described above for IVi, from tetrahydropyran-3,5-dione (IIc) and 5-chloro-2-iodo-benzoic acid (IIIf). LCMS: m/z found 250.2 [M+H]⁺; RT=0.76 min (Method B); ¹H NMR (400 MHz, DMSO-d₆) δ 12.30 (s, 1H), 9.04 (dd, 1H), 8.15 (dd, 1H), 7.88 (ddd, 1H), 4.81-4.76 (m, 2H), 4.30-4.25 (m, 2H).

### 8-Chloro-1-(methylamino)-1,2,4,5-tetrahydropyrano[3,4-c]isoquinolin-6-one (Vz)

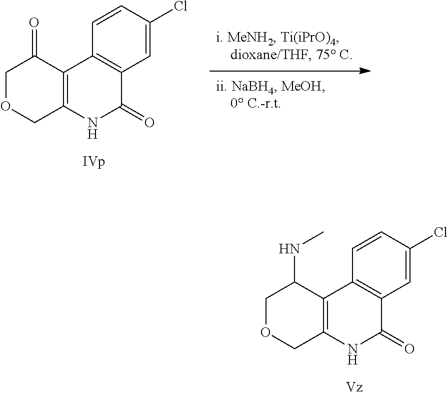 [SMILES: O=C1COCc2[nH]c(=O)c3cc(Cl)ccc3c21 ; CNC1COCc2[nH]c(=O)c3cc(Cl)ccc3c21]

8-Chloro-1-(methylamino)-1,2,4,5-tetrahydropyrano[3,4-c]isoquinolin-6-one was synthesized in an analogous manner as described above, from 8-chloro-4,5-dihydropyrano[3,4-c]isoquinoline-1,6-dione (IVp). LCMS: m/z found 234.1 [M−MeNH]; RT=0.49 min (Method B); ¹H NMR (400 MHz, CDCl₃) δ 11.84 (s, 1H), 8.31 (d, 1H), 7.69-7.58 (m, 2H), 4.69 (d, 1H), 4.58 (d, 1H), 4.42 (d, 1H), 3.64 (dd, 1H), 3.56 (s, 1H), 2.60 (s, 3H).

### 3-(3-Chloro-4-fluorophenyl)-1-(8-chloro-6-oxo-1,4,5,6-tetrahydro-2H-pyrano[3,4-c]isoquinolin-1-yl)-1-methylurea (Compound 55)

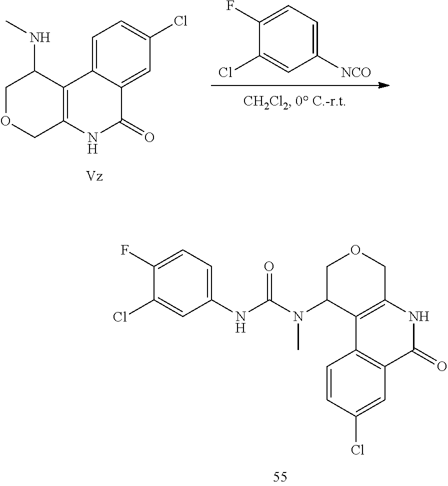

3-(3-Chloro-4-fluorophenyl)-1-(8-chloro-6-oxo-1,4,5,6-tetrahydro-2H-pyrano[3,4-c]isoquinolin-1-yl)-1-methylurea was synthesized in an analogous manner as described above, from 8-chloro-1-(methylamino)-1,2,4,5-tetrahydropyrano[3,4-c]isoquinolin-6-one (Vz). LCMS m/z found 436.1/438.2 [M+H]⁺; RT=4.36 min (Method A); ¹H NMR (400 MHz, DMSO-d₆) δ 11.63 (s, 1H), 8.57 (s, 1H), 8.15 (dd, 1H), 7.86 (ddd, 2H), 7.56-7.47 (m, 2H), 7.32 (td, 1H), 5.43 (s, 1H), 4.58 (d, 1H), 4.43 (dd, 1H), 4.05 (d, 1H), 3.93 (dd, 1H), 2.82-2.77 (s, 3H).

### 8-Chloro-1-(ethylamino)-1,2,4,5-tetrahydropyrano[3,4-c]isoquinolin-6-one (Vaa)

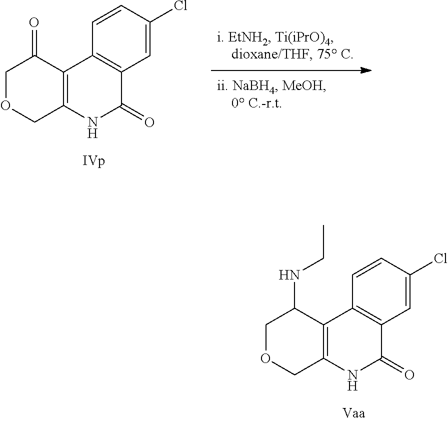 [SMILES: O=C1COCc2[nH]c(=O)c3cc(Cl)ccc3c21 ; CCNC1COCc2[nH]c(=O)c3cc(Cl)ccc3c21]

8-Chloro-1-(ethylamino)-1,2,4,5-tetrahydropyrano[3,4-c]isoquinolin-6-one was synthesized in an analogous manner as described above, from 8-chloro-4,5-dihydropyrano[3,4-c]isoquinoline-1,6-dione (IVp). LCMS: m/z found 279.3 [M+H]⁺; RT=0.52 min (Method B); ¹H NMR (400 MHz, CDCl₃) δ 8.20 (t, 1H), 7.56 (d, 2H), 4.46 (d, 1H), 4.36 (dd, 1H), 4.23 (dd, 1H), 3.83 (br s, exchangeable Hs), 3.63-3.49 (m, 2H), 2.81 (dq, 1H), 2.67 (dq, 1H), 1.07 (t, 3H).

### 3-(3-Chloro-4-fluorophenyl)-1-(8-chloro-6-oxo-1,4,5,6-tetrahydro-2H-pyrano[3,4-c]isoquinolin-1-yl)-1-ethylurea (Compound 56)

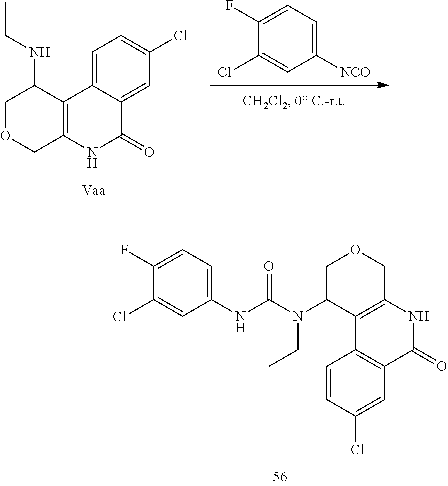

3-(3-Chloro-4-fluorophenyl)-1-(8-chloro-6-oxo-1,4,5,6-tetrahydro-2H-pyrano[3,4-c]isoquinolin-1-yl)-1-ethylurea was synthesized in an analogous manner as described above, from 8-chloro-1-(ethylamino)-1,2,4,5-tetrahydropyrano[3,4-c]isoquinolin-6-one (Vaa). LCMS m/z found 450.2/452.1 [M+H]⁺; RT=4.60 min (Method A); ¹H NMR (400 MHz, DMSO-d₆) δ 11.63 (s, 1H), 8.47 (s, 1H), 8.15 (d, 1H), 7.86 (ddd, 2H), 7.59-7.47 (m, 2H), 7.33 (t, 1H), 5.44 (d, 1H), 4.59 (d, 1H), 4.44 (dd, 1H), 4.03 (d, 1H), 3.91 (dd, 1H), 3.40 (dq, 1H), 3.33-3.13 (m, 1H), 0.84 (t, 3H).

### 1-(8-Chloro-6-oxo-1,4,5,6-tetrahydro-2H-pyrano[3,4-c]isoquinolin-1-yl)-3-(4-fluoro-3-methylphenyl)-1-methylurea (Compound 58)

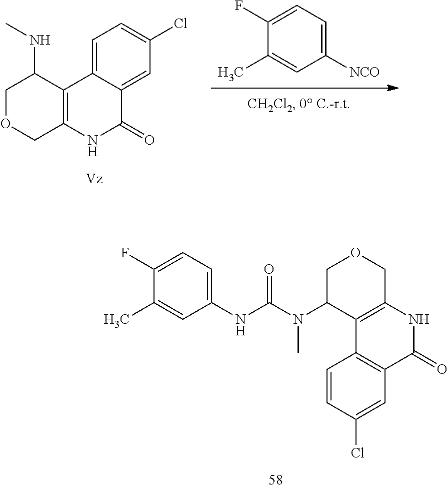

1-(8-Chloro-6-oxo-1,4,5,6-tetrahydro-2H-pyrano[3,4-c]isoquinolin-1-yl)-3-(4-fluoro-3-methylphenyl)-1-methylurea was synthesized in an analogous manner as described above, from 8-chloro-1-(methylamino)-1,2,4,5-tetrahydropyrano[3,4-c]isoquinolin-6-one (Vz). LCMS m/z found 416.2/418.2 [M+H]⁺; RT=4.07 min (Method A); ¹H NMR (400 MHz, DMSO-d₆) δ 11.62 (s, 1H), 8.33 (s, 1H), 8.15 (d, 1H), 7.83 (dd, 1H), 7.55 (d, 1H), 7.47 (dd, 1H), 7.40-7.31 (m, 1H), 7.03 (t, 1H), 5.44 (s, 1H), 4.58 (d, 1H), 4.42 (d, 1H), 4.03 (d, 1H), 3.92 (dd, 1H), 2.81-2.75 (m, 3H), 2.21 (d, 3H).

### 1-(8-Chloro-6-oxo-1,4,5,6-tetrahydro-2H-pyrano[3,4-c]isoquinolin-1-yl)-1-ethyl-3-(4-fluoro-3-methylphenyl)urea (Compound 59)

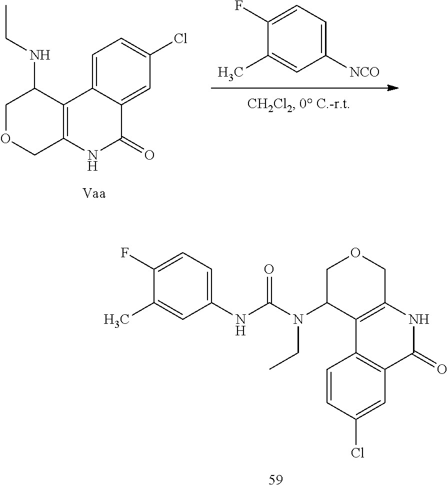

1-(8-Chloro-6-oxo-1,4,5,6-tetrahydro-2H-pyrano[3,4-c]isoquinolin-1-yl)-1-ethyl-3-(4-fluoro-3-methylphenyl)urea was synthesized in an analogous manner as described above, from 8-chloro-1-(ethylamino)-1,2,4,5-tetrahydropyrano[3,4-c]isoquinolin-6-one (Vaa). LCMS m/z found 430.2/432.3 [M+H]⁺; RT=4.31 min (Method A); ¹H NMR (400 MHz, DMSO-d₆) δ 11.62 (s, 1H), 8.24 (s, 1H), 8.15 (d, 1H), 7.83 (dd, 1H), 7.53 (d, 1H), 7.46 (dd, 1H), 7.37 (dt, 1H), 7.03 (t, 1H), 5.45 (s, 1H), 4.59 (d, 1H), 4.48-4.39 (m, 1H), 4.01 (d, 1H), 3.90 (dd, 1H), 3.45-3.36 (m, 1H), 3.32-3.16 (m, 1H), 2.22 (d, 3H), 0.84 (t, 3H).

### 1-(8-Chloro-6-oxo-1,4,5,6-tetrahydro-2H-pyrano[3,4-c]isoquinolin-1-yl)-3-(3-cyano-4-fluorophenyl)-1-methylurea (Compound 65)

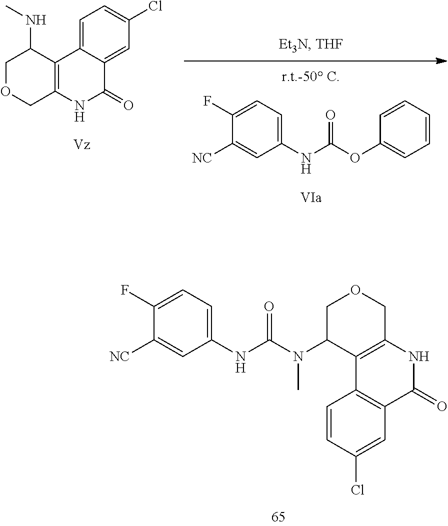 [SMILES: CNC1COCc2[nH]c(=O)c3cc(Cl)ccc3c21 ; N#Cc1cc(NC(=O)Oc2ccccc2)ccc1F ; CN(C(=O)Nc1ccc(F)c(C#N)c1)C1COCc2[nH]c(=O)c3cc(Cl)ccc3c21]

1-(8-Chloro-6-oxo-1,4,5,6-tetrahydro-2H-pyrano[3,4-c]isoquinolin-1-yl)-3-(3-cyano-4-fluorophenyl)-1-methylurea was synthesized in an analogous manner as described above for Compound 29, from 8-chloro-1-(methylamino)-1,2,4,5-tetrahydropyrano[3,4-c]isoquinolin-6-one (Vz) and phenyl N-(3-cyano-4-fluoro-phenyl)carbamate (VIa). LCMS m/z found 427.2/429.2 [M+H]⁺; RT=3.82 min (Method A); ¹H NMR (400 MHz, DMSO-d₆) δ 11.64 (s, 1H), 8.74 (s, 1H), 8.15 (d, 1H), 8.08 (d, 1H), 7.91-7.80 (m, 2H), 7.52 (d, 1H), 7.46 (t, 1H), 5.43 (s, 1H), 4.58 (d, 1H), 4.43 (d, 1H), 4.06 (d, 1H), 3.93 (dd, 1H), 2.80 (s, 3H).

### 1-(8-Chloro-6-oxo-1,4,5,6-tetrahydro-2H-pyrano[3,4-c]isoquinolin-1-yl)-3-(3-cyano-4-fluorophenyl-1-ethylurea Compound 66)

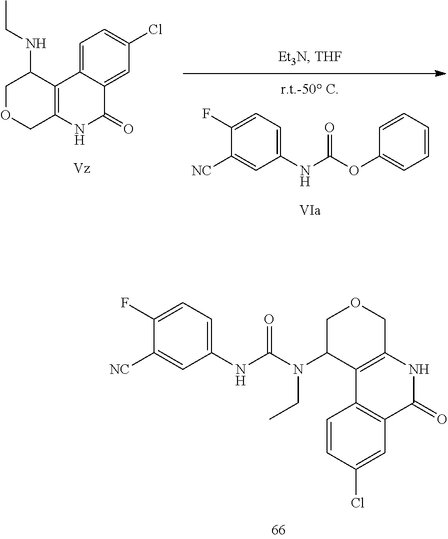 [SMILES: CCNC1COCc2[nH]c(=O)c3cc(Cl)ccc3c21 ; N#Cc1cc(NC(=O)Oc2ccccc2)ccc1F ; CCN(C(=O)Nc1ccc(F)c(C#N)c1)C1COCc2[nH]c(=O)c3cc(Cl)ccc3c21]

1-(8-Chloro-6-oxo-1,4,5,6-tetrahydro-2H-pyrano[3,4-c]isoquinolin-1-yl)-3-(3-cyano-4-fluorophenyl)-1-ethylurea was synthesized in an analogous manner as described above, from 8-chloro-1-(ethylamino)-1,2,4,5-tetrahydropyrano[3,4-c]isoquinolin-6-one (Vaa) and phenyl N-(3-cyano-4-fluoro-phenyl)carbamate (VIa). LCMS m/z found 441.2/443.2 [M+H]⁺; RT=4.03 min (Method A); ¹H NMR (400 MHz, DMSO-d₆) δ 11.64 (s, 1H), 8.67-8.61 (m, 1H), 8.12 (dd, 2H), 7.94-7.87 (m, 1H), 7.84 (d, 1H), 7.54-7.42 (m, 2H), 5.45 (s, 1H), 4.60 (d, 1H), 4.44 (d, 1H), 4.04 (d, 1H), 3.92 (dd, 1H), 3.41 (dd, 1H), 3.32-3.19 (m, 1H), 2.50 (t, 3H), 0.84 (t, 3H).

### 3,4,7,8,9,10-Hexahydrophenanthridine-1,6(2H,5H)-dione (IVq)

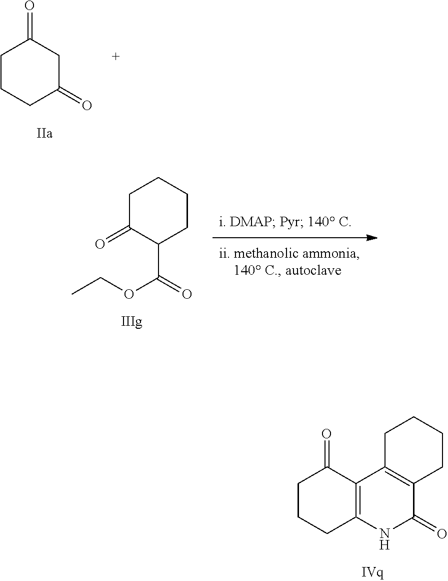 [SMILES: O=C1CCCC(=O)C1 ; CCOC(=O)C1CCCCC1=O ; O=C1CCCc2[nH]c(=O)c3c(c21)CCCC3]

Step i: To a stirred solution of 2.5 g (22.3 mmol) of cyclohexane-1,3-dione (Ha) in 7.5 mL of pyridine at room temperature under a nitrogen atmosphere were added 5.69 g (33.48 mmol) of ethyl 2-oxocyclohexane-1-carboxylate (Ig) followed by 54 mg (0.44 mmol) of 4-dimethylaminopyridine (DMAP). The mixture was then heated at 140° C. for 6 hours. Note: Reaction was performed on 4×2.5 g scales in parallel. All of the reaction mixtures were allowed to cool to room temperature, combined, diluted with water (500 mL) and extracted with ethyl acetate (2×500 mL). The combined organic extracts were washed with 1 M aqueous HCl (200 mL), brine (200 mL), dried over anhydrous Sodium sulfate and concentrated under reduced pressure. The obtained crude product was purified by silicagel column chromatography (eluted with a linear gradient of 10-20% ethyl acetate and petroleum ether). Pure fractions were concentrated under reduced pressure to afford 3.1 g (14.2 mmol, 16% overall yield) of 3,4,7,8,9,10-hexahydro-1H-benzo[c]chromene-1,6(2H)-dione. LCMS: m/z found 219.08 [M+H]⁺, RT=1.73 min, (Method A); ¹H NMR (400 MHz, CDCl₃): δ 2.97-2.95 (m, 2H), 2.86-2.82 (m, 2H), 2.55-2.45 (m, 4H), 2.11-2.05 (m, 2H), 1.71-1.68 (m, 4H).

Step ii: A stirred solution of 1.1 g (5.04 mmol) of 3,4,7,8,9,10-hexahydro-1H-benzo[c]chromene-1,6(2H)-dione, obtained in Step i, in 25 mL of 7 M methanolic ammonia in an autoclave was heated to 140° C. for 4 hours. The reaction mixture was allowed to cool to room temperature and concentrated under reduced pressure. The obtained residue was triturated with pentane (10 mL), filtered, and the solid dried under vacuum to afford 0.7 g (3.22 mmol, 63%) of 3,4,7,8,9,10-hexahydrophenanthridine-1,6(2H,5H)-dione. LCMS: m/z found 218.11 [M+H]⁺, RT=1.41 min, (Method: D); ¹H NMR (300 MHz, DMSO-d₆): 11.80 (br s, 1H), 2.92-2.88 (m, 2H), 2.77-2.73 (m, 2H), 2.42-2.37 (m, 2H), 2.34-2.30 (m, 2H), 1.96-1.87 (m, 2H), 1.60-1.56 (m, 4H).

### 1-(Methylamino)-1,3,4,5,7,8,9,10-octahydrophenanthridin-6(2H)-one (Vab)

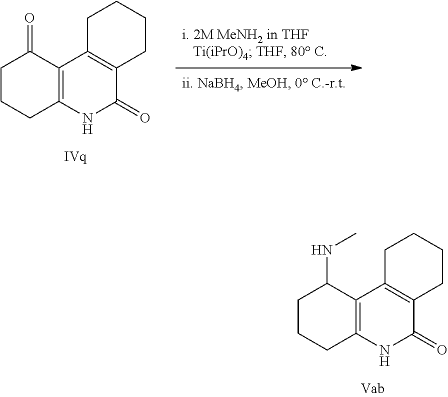 [SMILES: O=C1CCCc2[nH]c(=O)c3c(c21)CCCC3 ; CNC1CCCc2[nH]c(=O)c3c(c21)CCCC3]

To a solution of 0.3 g (1.38 mmol) of 3,4,7,8,9,10-hexahydrophenanthridine-1,6(2H,5H)-dione (IVq) in 3 mL of THE at room temperature under inert atmosphere were added 1.3 mL (2.60 mmol) of a 2 M methylamine solution in THE followed by 1.5 mL of titanium isopropoxide. The mixture was then heated at 80° C. for 6 hours. The reaction was cooled to 0° C., diluted with methanol (1.5 mL) and treated with 0.14 g (4.14 mmol) of sodium borohydride portion wise, and then stirred for at room temperature 2 hours. The mixture was then diluted with water (30 mL) and ethyl acetate (30 mL). The heterogeneous mixture was filtered and washed with ethyl acetate (10 mL). The organic layer was separated and the aqueous layer was extracted with ethyl acetate (2×40 mL). The combined organic extracts were washed with brine (50 mL), dried over anhydrous sodium sulfate and concentrated under reduced pressure to afford 250 mg of 1-(methylamino)-1,3,4,5,7,8,9,10-octahydrophenanthridin-6(2H)-one, which was carried directly to the next step. LCMS: m/z found 233.19 [M+H]⁺.

### 1-(Ethylamino)-1,3,4,5,7,8,9,10-octahydrophenanthridin-6(2H)-one (Vac)

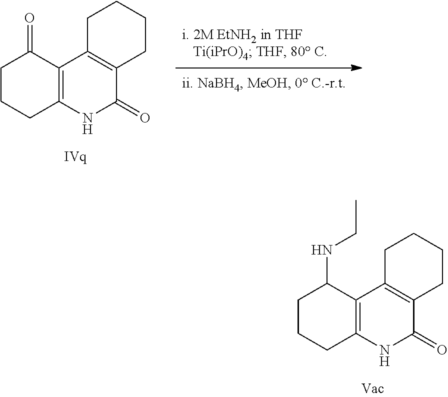 [SMILES: O=C1CCCc2[nH]c(=O)c3c(c21)CCCC3 ; CCNC1CCCc2[nH]c(=O)c3c(c21)CCCC3]

1-(Ethylamino)-1,3,4,5,7,8,9,10-octahydrophenanthridin-6(2H)-one was synthesized in an analogous manner as described above. LCMS: m/z found 247.15 [M+H]⁺, RT=1.04 min, (Method A); ¹H NMR (300 MHz, DMSO-d₆): 10.90 (br s, 1H), 3.46-3.44 (m, 1H), 2.89-2.81 (m, 1H), 2.72-2.63 (m, 1H), 2.51-2.26 (m, 5H), 2.00-1.92 (m, 1H), 1.87-1.73 (m, 1H), 1.68-1.52 (m, 5H), 1.30-11.19 (m, 3H), 1.01 (t, 3H).

### 3-(3-Chloro-4-fluorophenyl)-1-methyl-1-(6-oxo-1,2,3,4,5,6,7,8,9,10-decahydrophenanthridin-1-yl)urea (Compounds 46 and 47)

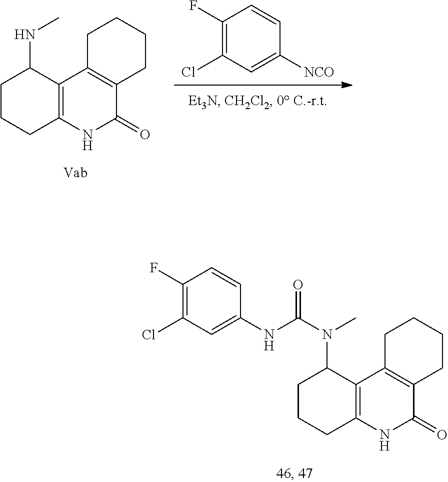

To a stirred solution of 0.16 g of 1-(methylamino)-1,2,3,4,7,8,9,10-octahydrophenanthridin-6(5H)-one (Vab) in 4 mL of dichloromethane at 0° C. were added 0.21 g (2.06 mmol) of triethylamine followed by 70 mg (0.41 mmol) of 2-chloro-1-fluoro-4-isocyanatobenzene and stirring was continued at room temperature for 2 hours. The reaction mixture was then diluted with water (50 mL), the precipitated solid collected by filtration. The solids were washed with pentane (10 mL) and dried under vacuum to afford 0.20 g (0.48 mmol, 54% overall yield in two steps) of racemic 3-(3-chloro-4-fluorophenyl)-1-methyl-1-(6-oxo-1,2,3,4,5,6,7,8,9,10-decahydrophenanthridin-1-yl)urea. The enantiomers were subsequently separated by preparative SFC: Method isocratic, Mobile phase MeOH: CO₂ —50:50. Column: Chiralpak IG (30×250 mm) 5 μm, flow rate: 90 g/min.

Enantiomer I (Compound 46): LCMS: m/z found 404.3/406.2 [M+H]⁺, RT=3.88 min (Method A); ¹H NMR (400 MHz, DMSO-d₆) δ 11.27 (br s, 1H), 8.38 (br s, 1H), 7.85-7.82 (m, 1H), 7.50-7.46 (m, 1H), 7.28 (t, 1H), 5.18-5.17 (m, 1H), 2.67 (s, 3H), 2.51-2.09 (m, 6H), 1.79-1.53 (m, 6H), 1.53-1.48 (m, 2H); Chiral analytical SFC: RT=2.16 min, Column: Chiralpak IG (250×4.6 mm, 5 m), 50% methanol, Flow rate: 4.0 ml/min.

Enantiomer II (Compound 47): LCMS: m/z found 404.2/406.2 [M+H]⁺, RT=3.88 min; (Method A); ¹H NMR (400 MHz, DMSO-d₆): δ 11.27 (br s, 1H), 8.39 (br s, 1H), 7.85-7.82 (m, 1H), 7.50-7.46 (m, 1H), 7.28 (t, 1H), 5.18-5.17 (m, 1H), 2.67 (s, 3H), 2.51-2.09 (m, 6H), 1.79-1.53 (m, 6H), 1.53-1.48 (m, 2H); Chiral analytical SFC: RT=7.76 min, Column: Chiralpak IG (250×4.6 mm) 5 μm, 50% methanol, Flow rate: 4.0 ml/min.

### 3-(3,4-Difluorophenyl)-1-methyl-1-(6-oxo-1,2,3,4,5,6,7,8,9,10-decahydrophenanthridin-1-yl)urea (Compounds 48 and 49)

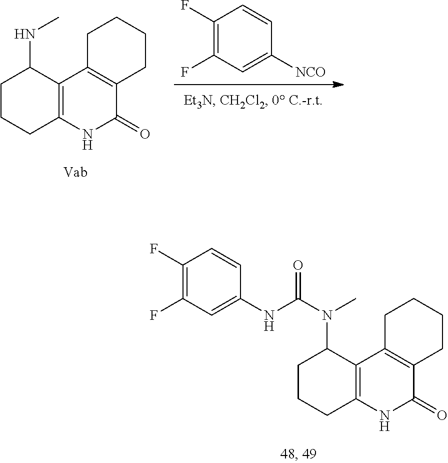

3-(3,4-Difluorophenyl)-1-methyl-1-(6-oxo-1,2,3,4,5,6,7,8,9,10-decahydrophenanthridin-1-yl) urea was synthesized in an analogous manner as described above, from 1,2-difluoro-4-isocyanatobenzene. The enantiomers were subsequently separated by preparative SFC: Method isocratic, Mobile phase MeOH: CO₂—55:45. Column: Chiralpak IG (30×250 mm) 5 μm, flow rate: 90 g/min.

Enantiomer I (Compound 48): LCMS: m/z found 388.3 [M+H]⁺, RT=3.55 min; (Method A); ¹H NMR (400 MHz, DMSO-d₆): δ 11.26 (br s, 1H), 8.39 (br s, 1H), 7.73-7.67 (m, 1H), 7.33-7.27 (m, 2H), 5.19-5.18 (m, 1H), 2.67 (s, 3H), 2.60-2.34 (m, 4H), 2.28-2.07 (m, 2H), 1.79-1.64 (m, 6H), 1.52-1.46 (m, 2H); Chiral analytical SFC: RT=3.62 min; (Column: Chiralpak IG-3 (4.6×150 mm) 3 μm, 0.5% Isopropyl Amine, 30% iso-propanol, Flow rate: 3.0 g/min.

Enantiomer II (Compound 49): LCMS: m/z found 388.3 [M+H]⁺, RT=3.55 min (Method A); ¹H NMR (400 MHz, DMSO-d₆): δ 11.26 (br s, 1H), 8.39 (br s, 1H), 7.73-7.67 (m, 1H), 7.33-7.27 (m, 2H), 5.19-5.18 (m, 1H), 2.67 (s, 3H), 2.60-2.34 (m, 4H), 2.28-2.07 (m, 2H), 1.79-1.64 (m, 6H), 1.52-1.46 (m, 2H); Chiral analytical SFC: RT=7.45 min; (Column: Chiralpak IG-3 (4.6×150 mm) 3 μm, 0.5% Isopropyl Amine, 30% iso-propanol, Flow rate: 3.0 g/min.

### 3-(3,4-Difluorophenyl)-1-ethyl-1-(6-oxo-1,2,3,4,5,6,7,8,9,10-decahydrophenanthridin-1-yl)urea (Compounds 50 and 51)

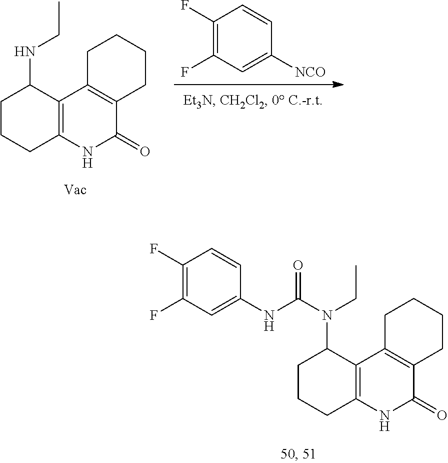

3-(3,4-Difluorophenyl)-1-ethyl-1-(6-oxo-1,2,3,4,5,6,7,8,9,10-decahydrophenanthridin-1-yl)urea was synthesized in an analogous manner as described above, from 1-(ethylamino)-1,3,4,5,7,8,9,10-octahydrophenanthridin-6(2H)-one (Vac) and 1,2-difluoro-4-isocyanatobenzene. The enantiomers were subsequently separated by preparative SFC: Method isocratic, Mobile phase MeOH: CO₂—30:70. Column: Lux Cellulose-2 (30×250 mm), 5μ, flow rate: 90 g/min.

Enantiomer I (Compound 50): LCMS: m/z found 402.3 [M+H]⁺, RT=3.78 min (Method A); ¹H NMR (400 MHz, DMSO-d₆): δ 11.27 (br s, 1H), 8.32 (br s, 1H), 7.77-7.67 (m, 1H), 7.32-7.25 (m, 2H), 5.19-5.18 (m, 1H), 3.23-3.06 (m, 2H), 2.61-2.25 (m, 6H), 1.79-1.52 (m, 8H), 0.91 (t, 3H); Chiral analytical SFC: RT=3.23 min; Column Lux Cellulose-2 (4.6×250 mm), 5μ, 40% methanol, Flow rate: 3.0 g/min.

Enantiomer II (Compound 51): LCMS: m/z found 402.3 [M+H]⁺, RT=3.78 min (Method A); ¹H NMR (400 MHz, DMSO-d₆): δ 11.27 (br s, 1H), 8.32 (br s, 1H), 7.77-7.67 (m, 1H), 7.32-7.25 (m, 2H), 5.19-5.18 (m, 1H), 3.23-3.06 (m, 2H), 2.61-2.25 (m, 6H), 1.79-1.52 (m, 8H), 0.91 (t, 3H); Chiral analytical SFC: RT=4.76 min; Column Lux Cellulose-2 (4.6×250 mm), 5μ, 40% methanol, Flow rate: 3.0 g/min.

### 3-(3-Chloro-4-fluorophenyl)-1-ethyl-1-(6-oxo-1,2,3,4,5,6,7,8,9,10-decahydrophenanthridin-1-yl)urea (Compounds 53 and 54)

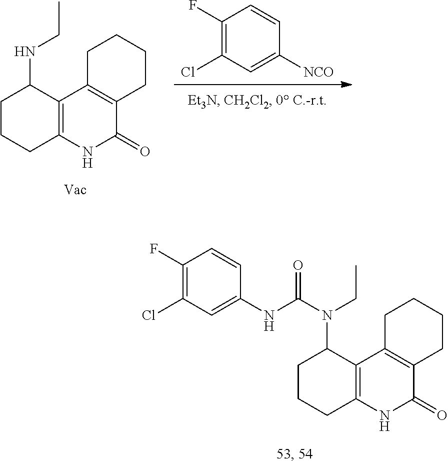

3-(3-Chloro-4-fluorophenyl)-1-ethyl-1-(6-oxo-1,2,3,4,5,6,7,8,9,10-decahydrophenanthridin-1-yl)urea was synthesized in an analogous manner as described above, from 1-(ethylamino)-1,3,4,5,7,8,9,10-octahydrophenanthridin-6(2H)-one (Vac) and 2-chloro-1-fluoro-4-isocyanatobenzene. The enantiomers were subsequently separated by preparative SFC: Method isocratic, Mobile phase MeOH: CO₂—45:55. Column: Chiralpak IG (30×250)mm, 5μ, flow rate: 100 g/min.

Enantiomer I (Compound 53): LCMS: m/z found 418.3/420.3 [M+H]⁺, RT=4.10 min (Method A); ¹H NMR (400 MHz, DMSO-d₆): δ 11.27 (br s, 1H), 8.32 (br s, 1H), 7.84-7.81 (m, 1H), 7.52-7.48 (m, 1H), 7.29 (t, 1H), 5.19-5.17 (m, 1H), 3.28-3.04 (m, 2H), 2.61-2.24 (m, 6H), 1.78-1.51 (m, 8H), 0.91 (t, 3H); Chiral analytical SFC: RT=1.94 min; Column CHIRALPAK IG-3 (4.6×150 mm) 3 μm, 45% methanol, Flow rate: 3.0 g/min.

Enantiomer I (Compound 54): LCMS: m/z found 418.3/420.3 [M+H]⁺, RT=4.09 min (Method A); ¹H NMR (400 MHz, DMSO-d₆): δ 11.27 (br s, 1H), 8.32 (br s, 1H), 7.84-7.81 (m, 1H), 7.52-7.48 (m, 1H), 7.29 (t, 1H), 5.19-5.17 (m, 1H), 3.28-3.04 (m, 2H), 2.61-2.24 (m, 6H), 1.78-1.51 (m, 8H), 0.91 (t, 3H); Chiral analytical SFC: RT=4.02 min; Column CHIRALPAK IG-3 (4.6×150 mm) 3 μm, 45% methanol, Flow rate: 3.0 g/min.

### 4-Bromo-5,6-dihydro-2H-pyran-3-carboxylic acid (IIIh)

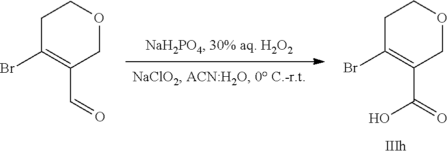 [SMILES: O=CC1=C(Br)CCOC1 ; O=C(O)C1=C(Br)CCOC1]

To a stirred solution of 2.9 g (15.18 mmol) of 4-bromo-5,6-dihydro-2H-pyran-3-carbaldehyde in 29 mL of 1:1 (v/v) acetonitrile:water at 0° C., were added 0.55 g (4.60 mmol) of sodium dihydrogen phosphate (NaH₂PO₄) and 5.8 mL of 30% aqueous H₂O₂ followed by 1.94 g (21.48 mmol) of sodium chlorite (NaClO₂). The reaction was allowed to stir at room temperature for 4 hours. The acetonitrile was evaporated under reduced pressure and the remaining solution was acidified with 1 M aqueous HCl (to pH˜4-5) and extracted with 10% methanol in dichloromethane (3×100 mL). The combined organic extracts were dried over anhydrous Sodium sulfate and concentrated under reduced pressure to afford 2.5 g (12.07 mmol, 79%) of 4-bromo-5,6-dihydro-2H-pyran-3-carboxylic acid. ¹H NMR (300 MHz, DMSO-d₆): δ 13.10 (br s, 1H), 4.24 (t, 2H), 3.73 (t, 2H), 2.67-2.61 (m, 2H).

### 1,2,4,7,8,9-Hexahydro-5H-pyrano[3,4-c]quinoline-5,10(6H)-dione (IVr)

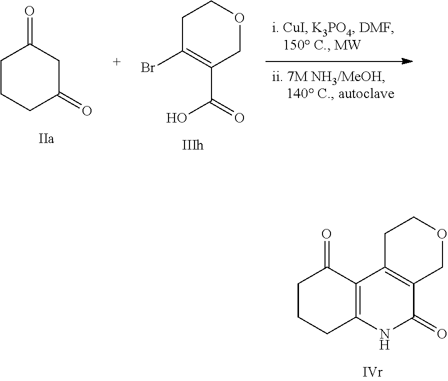 [SMILES: O=C1CCCC(=O)C1 ; O=C(O)C1=C(Br)CCOC1 ; O=C1CCCc2[nH]c(=O)c3c(c21)CCOC3]

Step i: A microwave tube was charged with a solution of 1.0 g (4.85 mmol) of 4-bromo-5,6-dihydro-2H-pyran-3-carboxylic acid (IIIh) in 10 mL of DMF, 0.82 g (7.28 mmol) of cyclohexane-1,3-dione (Ha) and 2.06 g (9.70 mmol) of K₃PO₄ and the mixture was degassed with nitrogen for 5 min. Copper(I)iodide (0.37 g, 1.94 mmol) was added, the vessel sealed and the mixture was subjected to microwave irradiation, maintaining a reaction temperature of 150° C. for 1 hours. Note: Reaction was performed in triplicate on 1.0 g scale. The triplicate reaction mixtures were combined and passed through a silicagel plug (eluted with 40-50% linear gradient of ethyl acetate and petroleum ether). The filtrate was concentrated under reduced pressure and the product was purified by silicagel column chromatography (eluted with 25-35% linear gradient of ethyl acetate and petroleum ether) to afford 0.85 g (3.86 mmol, 26%) of 1,2,4,7,8,9-hexahydro-5H,10H-pyrano[3,4-c]chromene-5,10-dione. LCMS: m/z found 221.04 [M+H]⁺, RT=1.39 min (Method A); ¹H NMR (400, CDCl₃) δ 4.49-4.48 (m, 2H), 3.84 (t, 2H), 3.09-3.06 (m, 2H), 2.89-2.86 (m, 2H), 2.57-2.54 (m, 2H), 2.15-2.08 (m, 2H).

Step ii: An autoclave was charged with 1.1 g (4.31 mmol) of 1,2,4,7,8,9-hexahydro-5H,10H-pyrano[3,4-c]chromene-5,10-dione obtained in Step i and 15 mL of 7 M methanolic ammonia, and the reaction mixture was stirred at 140° C. for 4 hours. The mixture was allowed to cool to room temperature and concentrated under reduced pressure. The residue was triturated with pentane (10 mL), the solid filtered and dried under vacuum to afford 0.81 g (3.69 mmol, 85%) of 1,2,4,7,8,9-hexahydro-5H-pyrano[3,4-c]quinoline-5,10(6H)-dione. LCMS: m/z found 220.07 [M+H]⁺, RT=1.17 min, (Method A); ¹H NMR (300 MHz, DMSO-d₆): δ 9.28 (br s, 1H), 4.34-4.32 (m, 2H), 3.73-3.69 (m, 2H), 2.97-2.93 (m, 2H), 2.81-2.77 (m, 2H), 2.44-2.39 (m, 2H), 1.98-1.92 (m, 2H).

### 10-(Methylamino)-1,2,4,6,7,8,9,10-octahydro-5H-pyrano[3,4-c]quinolin-5-one (Vad)

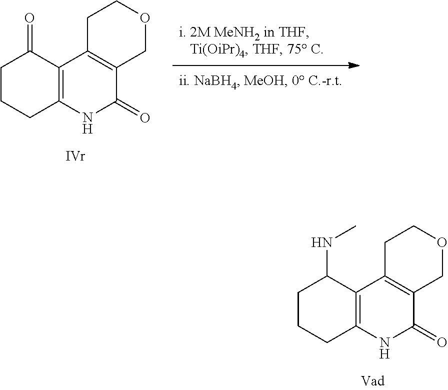 [SMILES: O=C1CCCc2[nH]c(=O)c3c(c21)CCOC3 ; CNC1CCCc2[nH]c(=O)c3c(c21)CCOC3]

10-(Methylamino)-1,2,4,6,7,8,9,10-octahydro-5H-pyrano[3,4-c]quinolin-5-one was synthesized in an analogous manner as described above, from 1,2,4,7,8,9-Hexahydro-5H-pyrano[3,4-c]quinoline-5,10(6H)-dione (IVr) and methylamine. LCMS: m/z found 235.14 [M+H]⁺, RT=0.32 min (Method A).

### 3-(3-Chloro-4-fluorophenyl)-1-methyl-1-(5-oxo-1,4,5,6,7,8,9,10-octahydro-2H-pyrano[3,4-c]quinolin-10-yl)urea (Compounds 60 and 61)

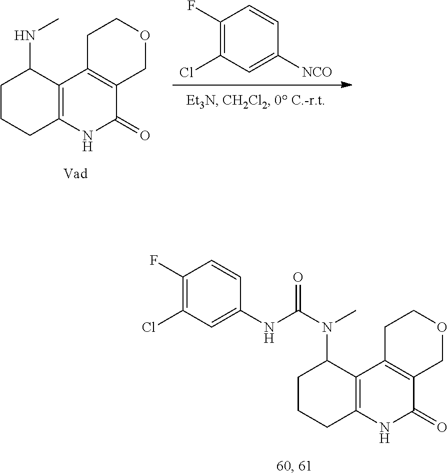

3-(3-Chloro-4-fluorophenyl)-1-methyl-1-(5-oxo-1,4,5,6,7,8,9,10-octahydro-2H-pyrano[3,4-c]quinolin-10-yl)urea was synthesized in an analogous manner as described above, from 10-(methylamino)-1,2,4,6,7,8,9,10-octahydro-5H-pyrano[3,4-c]quinolin-5-one (Vad) and 2-chloro-1-fluoro-4-isocyanatobenzene. The enantiomers were subsequently separated by preparative SFC: Method isocratic, Mobile phase MeOH: CO₂—50:50. Column: Chiralpak IC (30×250 mm), 5μ, flow rate: 90 g/min.

Enantiomer I (Compound 60): LCMS: m/z found 406.2/408.2 [M+H]⁺, RT=3.24 min (Method A); ¹H NMR (400 MHz, DMSO-d₆): δ 11.47 (br s, 1H), 8.40 (br s, 1H), 7.83-7.81 (m, 1H), 7.49-7.45 (m, 1H), 7.29 (t, 1H), 5.23-5.21 (m, 1H), 4.40 (d, 1H), 4.27 (d, 1H), 3.90-3.85 (m, 1H), 3.59-3.53 (m, 1H), 2.65-2.56 (m, 4H), 2.49-2.31 (m, 3H), 1.78-7.65 (m, 4H); Chiral analytical SFC: RT=2.74 min, Column: Chiralpak IC-3 (4.6×150 mm) 3.5 μm, 40% methanol, Flow rate: 3 g/min.

Enantiomer II (Compound 61): LCMS: m/z found 406.3/408.3 [M+H]⁺, RT=3.24 min, (Method A); ¹H NMR (400 MHz, DMSO-d₆): δ 11.47 (br s, 1H), 8.40 (br s, 1H), 7.83-7.81 (m, 1H), 7.49-7.45 (m, 1H), 7.29 (t, 1H), 5.23-5.21 (m, 1H), 4.40 (d, 1H), 4.27 (d, 1H), 3.90-3.85 (m, 1H), 3.59-3.53 (m, 1H), 2.65-2.56 (m, 4H), 2.49-2.31 (m, 3H), 1.78-7.65 (m, 4H); Chiral analytical SFC: RT=5.19 min, Column: Chiralpak IC-3 (4.6×150 mm) 3.5 μm, 40% methanol, Flow rate: 3 g/min.

### 3-(3,4-Difluorophenyl)-1-methyl-1-(5-oxo-1,4,5,6,7,8,9,10-octahydro-2H-pyrano[3,4-c]quinolin-10-yl)urea (Compounds 62 and 63)

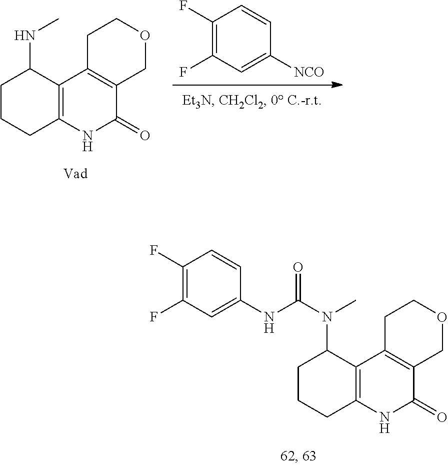

3-(3,4-Difluorophenyl)-1-methyl-1-(5-oxo-1,4,5,6,7,8,9,10-octahydro-2H-pyrano[3,4-c]quinolin-10-yl)urea was synthesized in an analogous manner as described above, from 10-(methylamino)-1,2,4,6,7,8,9,10-octahydro-5H-pyrano[3,4-c]quinolin-5-one (Vad) and 1,2-difluoro-4-isocyanatobenzene. The enantiomers were subsequently separated by preparative SFC: Method isocratic, Mobile phase MeOH: CO₂—50:50. Column: Chiralpak IC (30×250 mm), 5μ, flow rate: 90 g/min.

Enantiomer I (Compound 62): LCMS: m/z found 390.3 [M+H]⁺, RT=2.89 min, (Method A); ¹H NMR (400 MHz, DMSO-d₆): δ 11.47 (br s, 1H), 8.41 (br s, 1H), 7.72-7.66 (m, 1H), 7.33-7.27 (m, 2H), 5.23-5.21 (m, 1H), 4.40 (d, 1H), 4.27 (d, 1H), 3.89-3.85 (m, 1H), 3.57-3.54 (m, 1H), 2.66-2.56 (m, 4H), 2.50-2.32 (m, 3H), 1.82-1.65 (m, 4H); Chiral analytical SFC: RT=2.21 min, Column: Chiralpak IC-3 (4.6×150 mm) 3.5 μm, 40% methanol, Flow rate: 3 g/min.

Enantiomer II (Compound 63): LCMS: m/z found 390.3 [M+H]⁺, RT=2.89 min, (Method A); ¹H NMR (400 MHz, DMSO-d₆): δ 11.47 (br s, 1H), 8.41 (br s, 1H), 7.72-7.66 (m, 1H), 7.33-7.27 (m, 2H), 5.23-5.21 (m, 1H), 4.40 (d, 1H), 4.27 (d, 1H), 3.89-3.85 (m, 1H), 3.57-3.54 (m, 1H), 2.66-2.56 (m, 4H), 2.50-2.32 (m, 3H), 1.82-1.65 (m, 4H); Chiral analytical SFC: RT=3.96 min, Column: Chiralpak IC-3 (4.6×150 mm) 3.5 μm, 40% methanol, Flow rate: 3 g/min.

### 3,4,8,9-Tetrahydro-1H-pyrano[4,3-c]quinoline-5,10(6H,7H)-dione (IVs)

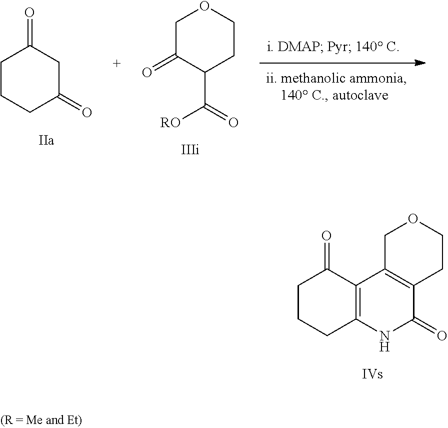

3,4,8,9-Tetrahydro-1H-pyrano[4,3-c]quinoline-5,10(6H,7H)-dione was synthesized in an analogous manner as described above for IVq, from a ˜1:1 mixture of methyl 3-oxotetrahydro-2H-pyran-4-carboxylate/ethyl 3-oxotetrahydro-2H-pyran-4-carboxylate (IIIi) and cyclohexane-1,3-dione (IIa). LCMS: m/z found 220.13 [M+H]⁺; ¹H NMR (400 MHz, DMSO-d₆): δ 11.98 (br s, 1H), 4.78 (s, 2H), 3.74 (t, 2H), 2.80-2.76 (m, 2H), 2.45-2.38 (m, 4H), 1.97-1.91 (m, 2H).

### 10-(Methylamino)-3,4,7,8,9,10-hexahydro-1H-pyrano[4,3-c]quinolin-5(6H)-one (Vae)

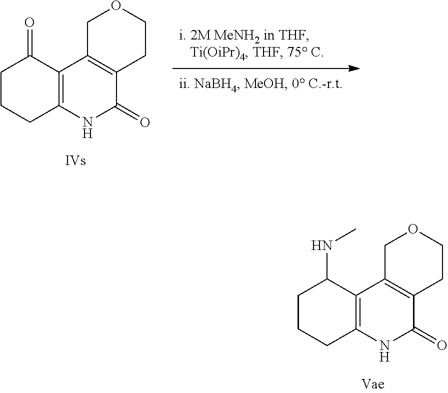 [SMILES: O=C1CCCc2[nH]c(=O)c3c(c21)COCC3 ; CNC1CCCc2[nH]c(=O)c3c(c21)COCC3]

10-(Methylamino)-3,4,7,8,9,10-hexahydro-1H-pyrano[4,3-c]quinolin-5(6H)-one was synthesized in an analogous manner as described above, from 3,4,8,9-tetrahydro-1H-pyrano[4,3-c]quinoline-5,10(6H,7H)-dione (IVs) and methylamine. LCMS: m/z found 235.14 [M+H]⁺, RT=0.29 min (Method A); 1H NMR (400 MHz, DMSO-d₆): δ 11.23 (br s, 1H), 4.81 (d, 1H), 4.51 (d, 1H), 3.76 (t, 2H), 2.43-2.31 (m, 8H), 2.03-1.97 (m, 1H), 1.86-1.62 (m, 2H), 1.63-1.54 (m, 1H), 1.40-1.29 (m, 1H).

### 3-(3-Chloro-4-fluorophenyl)-1-methyl-1-(5-oxo-3,4,5,6,7,8,9,10-octahydro-1H-pyrano[4,3-c]quinolin-10-yl)urea (Compounds 79 and 80)

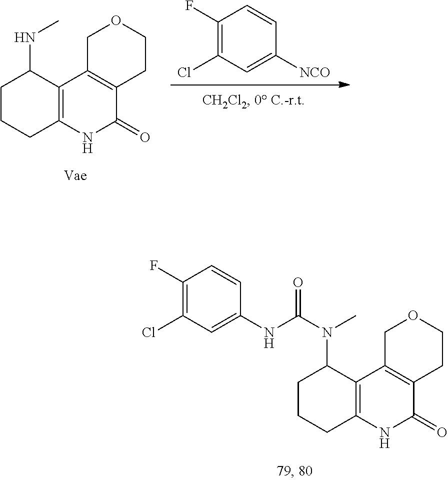

To a stirred solution of 100 mg (0.51 mmol) of 10-(methylamino)-3,4,7,8,9,10-hexahydro-1H-pyrano[4,3-c]quinolin-5(6H)-one (Vae) in 10 mL of dichloromethane at 0° C. was added 43 mg (0.25 mmol) of 1-fluoro-2-chloro-4-isocyanatobenzene and allowed to stir at room temperature for 1 hours. The reaction mixture was diluted with water (50 mL) and extracted with 10% methanol in dichloromethane (2×100 mL). The combined organic layer was washed with brine (30 mL), dried over anhydrous Sodium sulfate and concentrated under reduced pressure. The residue was triturated with diethyl ether (20 mL) at room temperature, the solid filtered and dried under vacuum to afford 120 mg (0.29 mmol, 69%) of 3-(3-chloro-4-fluorophenyl)-1-methyl-1-(5-oxo-3,4,5,6,7,8,9,10-octahydro-1H-pyrano[4,3-c]quinolin-10-yl)urea. The enantiomers were subsequently separated by preparative SFC: Method isocratic, Mobile phase MeOH: CO₂—60:40. Column: Chiralpak IG (30×250 mm), 5μ, flow rate: 90 g/min.

Enantiomer I (Compound 79): LCMS: m/z found 406.3/408.3 [M+H]⁺, RT=3.23 min (Method A); ¹H NMR (400 MHz, DMSO-d₆): δ 11.50 (br s, 1H), 8.42 (br s, 1H), 7.83-7.81 (m, 1H), 7.50-7.46 (m, 1H), 7.30 (t, 1H), 5.17-5.16 (m, 1H), 4.36 (d, 1H), 4.10 (d, 1H), 3.95-3.89 (m, 1H), 3.64-3.58 (m, 1H), 2.67-2.50 (m, 5H), 2.49-2.37 (m, 2H), 1.84-1.61 (m, 4H); Chiral analytical SFC: RT=3.69 min; Column: Chiralpak IG-3 (4.6×150 mm) 3 μm, 40% methanol, Flow rate: 3.0 g/min.

Enantiomer II (Compound 80): LCMS: m/z found 406.3/408.3 [M+H]⁺, RT=3.23 min (Method A); ¹H NMR (400 MHz, DMSO-d₆): δ 11.50 (br s, 1H), 8.42 (br s, 1H), 7.83-7.81 (m, 1H), 7.50-7.46 (m, 1H), 7.30 (t, 1H), 5.17-5.16 (m, 1H), 4.36 (d, 1H), 4.10 (d, 1H), 3.95-3.89 (m, 1H), 3.64-3.58 (m, 1H), 2.67-2.50 (m, 5H), 2.49-2.37 (m, 2H), 1.84-1.61 (m, 4H); Chiral analytical SFC: RT=5.47 min; Column: Chiralpak IG-3 (4.6×150 mm) 3 μm, 40% methanol, Flow rate: 3.0 g/min.

### 3-(3,4-Difluorophenyl)-1-methyl-1-(5-oxo-3,4,5,6,7,8,9,10-octahydro-1H-pyrano[4,3-c]quinolin-10-yl)urea (Compounds 81 and 82)

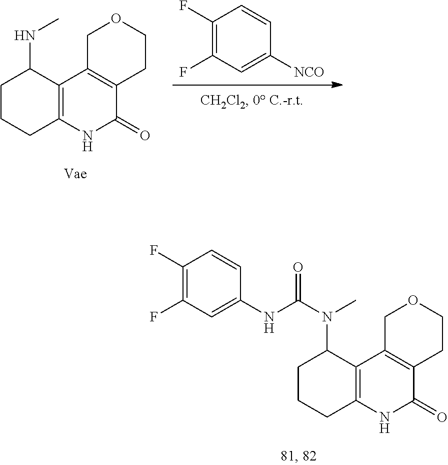

3-(3,4-Difluorophenyl)-1-methyl-1-(5-oxo-3,4,5,6,7,8,9,10-octahydro-1H-pyrano[4,3-c]quinolin-10-yl)urea was synthesized in an analogous manner as described above, from 10-(methylamino)-3,4,7,8,9,10-hexahydro-1H-pyrano[4,3-c]quinolin-5(6H)-one (Vae) and 1,2-difluoro-4-isocyanatobenzene. The enantiomers were subsequently separated by preparative SFC: Method isocratic, Mobile phase MeOH: CO₂—50:50. Column: Chiralpak IG (30×250 mm), 5μ, flow rate: 90 g/min.

Enantiomer I (Compound 81): LCMS: m/z found 390.3 [M+H]⁺, RT=2.89 min, (Method A); ¹H NMR (400 MHz, DMSO-d₆): δ 11.44 (br s, 1H), 8.43 (br s, 1H), 7.72-7.66 (m, 1H), 7.34-7.26 (m, 2H), 5.17-5.16 (m, 1H), 4.36 (d, 1H), 4.10 (d, 1H), 3.95-3.89 (m, 1H), 3.64-3.58 (m, 1H), 2.66-2.51 (m, 5H), 2.49-2.37 (m, 2H), 1.82-1.61 (m, 4H); Chiral analytical SFC: RT=2.93 min, Column: Chiralpak IG-3 (4.6×150 mm) 3 μm, 40% methanol, Flow rate: 3.0 g/min.

Enantiomer II (Compound 82): LCMS: m/z found 390.3 [M+H]⁺, RT=2.88 min, (Method A); ¹H NMR (400 MHz, DMSO-d₆): δ 11.44 (br s, 1H), 8.43 (br s, 1H), 7.72-7.66 (m, 1H), 7.34-7.26 (m, 2H), 5.17-5.16 (m, 1H), 4.36 (d, 1H), 4.10 (d, 1H), 3.95-3.89 (m, 1H), 3.64-3.58 (m, 1H), 2.66-2.51 (m, 5H), 2.49-2.37 (m, 2H), 1.82-1.61 (m, 4H); Chiral analytical SFC: RT=6.21 min, Column: Chiralpak IG-3 (4.6×150 mm) 3 μm, 40% methanol, Flow rate: 3.0 g/min.

### 4,7-Dihydro-1H,3H-dipyrano[3,4-b:3′,4′-d]pyridine-5,10(6H,9H)-dione (IVt)

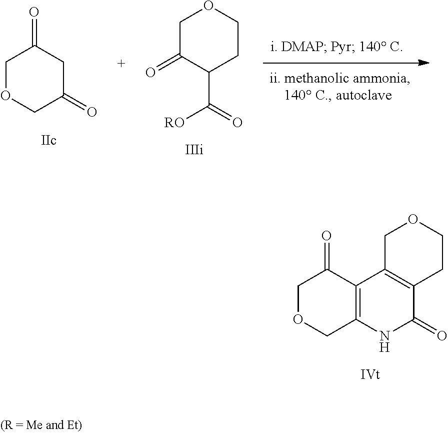

4,7-Dihydro-1H,3H-dipyrano[3,4-b:3′,4′-d]pyridine-5,10(6H,9H)-dione was synthesized in an analogous manner as described above, from a ˜1:1 mixture of methyl 3-oxotetrahydro-2H-pyran-4-carboxylate/ethyl 3-oxotetrahydro-2H-pyran-4-carboxylate (IIIi) and tetrahydropyran-3,5-dione (IIc). LCMS: m/z found 222.12 [M+H]⁺, RT=1.12 min, (Method A); ¹H NMR (300 MHz, DMSO-d₆): δ 11.02 (br s, 1H), 4.78-4.68 (m, 4H), 4.16-4.12 (m, 2H), 3.80-3.76 (m, 2H), 2.43-2.39 (m, 2H).

### 10-(Methylamino)-4,7,9,10-tetrahydro-1H,3H-dipyrano[3,4-b:3′,4′-d]pyridin-5(6H)-one (Vaf)

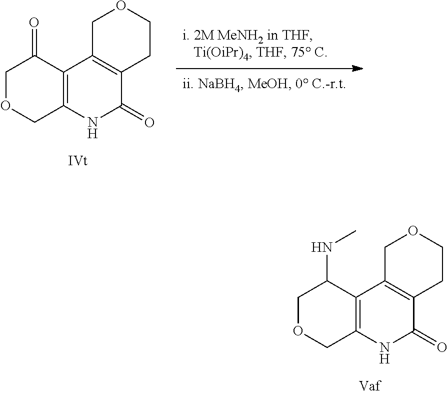 [SMILES: O=C1COCc2[nH]c(=O)c3c(c21)COCC3 ; CNC1COCc2[nH]c(=O)c3c(c21)COCC3]

10-(Methylamino)-4,7,9,10-tetrahydro-1H,3H-dipyrano[3,4-b:3′,4′-d]pyridin-5(6H)-one was synthesized in an analogous manner as described above, from 4,7-dihydro-1H,3H-dipyrano[3,4-b:3′,4′-d]pyridine-5,10(6H,9H)-dione (IVt) and methylamine. LCMS: m/z found 237.13 [M+H]⁺.

### 3-(3-Chloro-4-fluorophenyl)-1-methyl-1-(5-oxo-4,5,6,7,9,10-hexahydro-1H,3H-dipyrano[3,4-b:3′,4′-d]pyridin-10-yl)urea (Compounds 83 and 84)

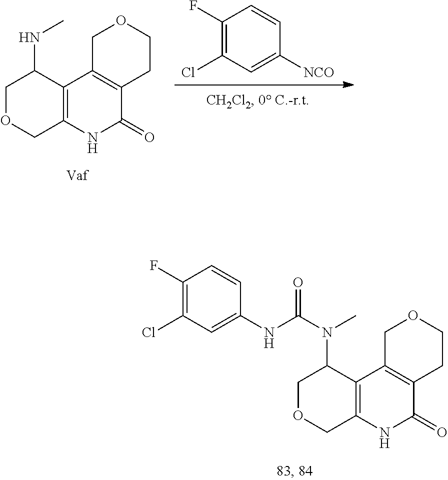

3-(3-Chloro-4-fluorophenyl)-1-methyl-1-(5-oxo-4,5,6,7,9,10-hexahydro-1H,3H-dipyrano[3,4-b:3′,4′-d]pyridin-10-yl)urea was synthesized in an analogous manner as described above, from 10-(methylamino)-4,7,9,10-tetrahydro-1H,3H-dipyrano[3,4-b:3′,4′-d]pyridin-5(6H)-one (Vaf) and 2-chloro-1-fluoro-4-isocyanatobenzene. The enantiomers were subsequently separated by preparative SFC: Method isocratic, Mobile phase MeOH: CO₂— 40:60. Column: Chiralpak IG (30×250 mm), 5μ, flow rate: 90 g/min.

Enantiomer I (Compound 83): LCMS: m/z found 408.3/410.3 [M+H]⁺, RT=2.96 min, (Method A); ¹H NMR (400 MHz, DMSO-d₆): δ 11.50 (br s, 1H), 8.52 (br s, 1H), 7.82-7.79 (m, 1H), 7.49-7.45 (m, 1H), 7.30 (t, 1H), 5.03-5.02 (m, 1H), 4.50 (d, 1H), 4.45 (d, 1H), 4.33 (d, 1H), 4.15 (d, 1H), 3.94-3.85 (m, 2H), 3.81-3.76 (m, 1H), 3.71-3.65 (m, 2H), 2.79 (s, 3H), 2.41-2.37 (br m, 2H); Chiral analytical SFC: RT=2.94 min, Column: Chiralpak IG-3 (4.6×150 mm) 3 μm, 35% methanol, Flow rate: 3.0 g/min.

Enantiomer II (Compound 84): LCMS: m/z found 408.3/410.2 [M+H]⁺, RT=2.96 min, (Method A); ¹H NMR (400 MHz, DMSO-d₆): δ 11.50 (br s, 1H), 8.52 (br s, 1H), 7.82-7.79 (m, 1H), 7.49-7.45 (m, 1H), 7.30 (t, 1H), 5.03-5.02 (m, 1H), 4.50 (d, 1H), 4.45 (d, 1H), 4.33 (d, 1H), 4.15 (d, 1H), 3.94-3.85 (m, 2H), 3.81-3.76 (m, 1H), 3.71-3.65 (m, 2H), 2.79 (s, 3H), 2.41-2.37 (br m, 2H); Chiral analytical SFC: RT=5.93 min, Column: Chiralpak IG-3 (4.6×150 mm) 3 μm, 35% methanol, Flow rate: 3.0 g/min.

### 3-(3,4-Difluorophenyl)-1-methyl-1-(5-oxo-1,3,4,5,6,7,9,10-octahydrodipyrano[3,4-b:3′,4′-d]pyridin-10-yl)urea (Compounds 85 and 86)

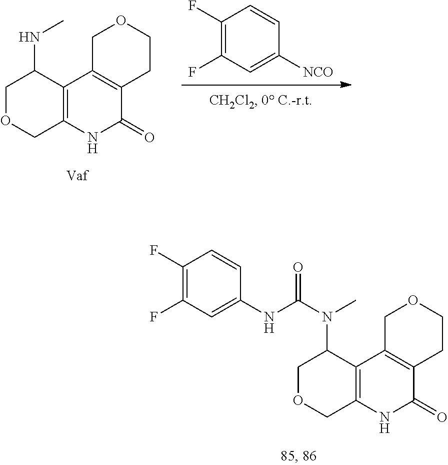

3-(3,4-Difluorophenyl)-1-methyl-1-(5-oxo-1,3,4,5,6,7,9,10-octahydrodipyrano[3,4-b:3′,4′-d]pyridin-10-yl)urea was synthesized in an analogous manner as described above, from 10-(methylamino)-4,7,9,10-tetrahydro-1H,3H-dipyrano[3,4-b:3′,4′-d]pyridin-5(6H)-one (Vaf) and 1,2-difluoro-4-isocyanatobenzene. The enantiomers were subsequently separated by preparative SFC: Method isocratic, Mobile phase MeOH: CO₂—35:65. Column: (R, R) WHELK-01 (30×250 mm), 5μ, flow rate: 90 g/min.

Enantiomer I (Compound 85): LCMS: m/z found 392.3 [M+H]⁺, RT=2.60 min, (Method A); ¹H NMR (400 MHz, DMSO-d₆): δ 11.50 (br s, 1H), 8.53 (br s, 1H), 7.70-7.65 (m, 1H), 7.35-7.29 (m, 2H), 5.03-5.01 (m, 1H), 4.50 (d, 1H), 4.44 (d, 1H), 4.33 (d, 1H), 4.15 (d, 1H), 3.94-3.85 (m, 2H), 3.81-3.77 (m, 1H), 3.71-3.66 (m, 1H), 2.79 (s, 3H), 2.39-2.37 (m, 2H); Chiral analytical SFC: RT=2.04 min; Column: (R,R) WHELK-01(4.6×150 mm) 3.5 m, 35% methanol, Flow rate: 3.0 g/min.

Enantiomer I (Compound 86): LCMS: m/z found 392.3 [M+H]⁺, RT=2.60 min, (Method A); ¹H NMR (400 MHz, DMSO-d₆): δ 11.50 (br s, 1H), 8.53 (br s, 1H), 7.70-7.65 (m, 1H), 7.35-7.29 (m, 2H), 5.03-5.01 (m, 1H), 4.50 (d, 1H), 4.44 (d, 1H), 4.33 (d, 1H), 4.15 (d, 1H), 3.94-3.85 (m, 2H), 3.81-3.77 (m, 1H), 3.71-3.66 (m, 1H), 2.79 (s, 3H), 2.39-2.37 (m, 2H); Chiral analytical SFC: RT=2.75 min; Column: (R,R) WHELK-01 (4.6×150 mm) 3.5 m, 35% methanol, Flow rate: 3.0 g/min.

### 5,7,9,10-Tetrahydrodipyrano[3,4-b:4′,3′-d]pyridine-1,6(2H,4H)-dione (IVu)

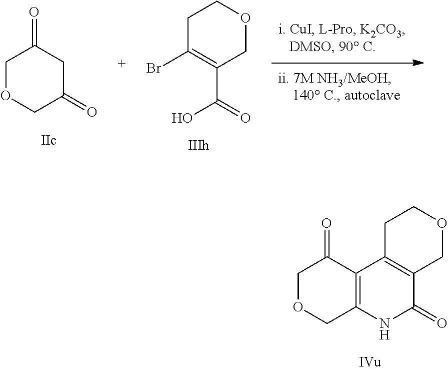 [SMILES: O=C1COCC(=O)C1 ; O=C(O)C1=C(Br)CCOC1 ; O=C1COCc2[nH]c(=O)c3c(c21)CCOC3]

Step i: To a stirred solution of 0.5 g (2.42 mmol) of 4-bromo-5,6-dihydro-2H-pyran-3-carboxylic acid (IIIh) in 5 mL of dry DMSO, were added 0.83 g (7.28 mmol) of 2H-pyran-3,5(4H,6H)-dione (IIc), 1.34 g (9.70 mmol) of potassium carbonate, 46 mg (0.29 mmol) of copper(I)iodide, and 56 mg (0.48 mmol) of L-proline. The reaction mixture was purged with argon for 5 min and stirred in a pre-heated oil bath at 90° C. for 2.5 hours. Note: The above detailed reaction was performed in duplicate on 0.5 g scale each. The reaction mixtures were combined and acidified with 2 M aqueous HCl (30 mL). The resulting solution was extracted with ethyl acetate (3×50 mL), the combined organic extracts washed with brine (50 mL), dried over anhydrous Sodium sulfate and concentrated under reduced pressure. The compound was isolated by silicagel column chromatography (eluted with 40-45% linear gradient of ethyl acetate and petroleum ether) to afford 0.65 g of 4,7,9,10-tetrahydro-6H-dipyrano[3,4-b:4′,3′-d]pyran-1,6(2H)-dione, which was taken the in next step. LCMS: m/z found 223.13 [M+H]⁺, RT=1.31 min, (Method A).

Step ii: An autoclave was charged with 0.65 g of 4,7,9,10-tetrahydro-6H-dipyrano[3,4-b:4′,3′-d]pyran-1,6(2H)-dione obtained in Step i and 20 mL of 7 M methanolic ammonia. The reaction mixture was then stirred at 140° C. for 4 hours. The mixture was allowed to cool to room temperature and concentrated under reduced pressure. The obtained residue was triturated with 1:1 (v/v) ethanol:n-pentane (15 mL), the solid was filtered and then dried under vacuum to afford 0.4 g (1.80 mmol, 37% over two steps) of 5,7,9,10-tetrahydrodipyrano[3,4-b:4′,3′-d]pyridine-1,6(2H,4H)-dione (IVu). LCMS: m/z found 222.11 [M+H]⁺, RT=1.48 min, (Method A); ¹H NMR (300 MHz, DMSO-d₆): δ 11.47 (br s, 1H), 4.69 (s, 2H), 4.36 (s, 2H), 4.16 (s, 2H), 3.77-3.73 (m, 2H), 2.98-2.94 (m, 2H).

### 1-(Methylamino)-1,2,5,7,9,10-hexahydrodipyrano[3,4-b:4′,3′-d]pyridin-6(4H)-one (Vag)

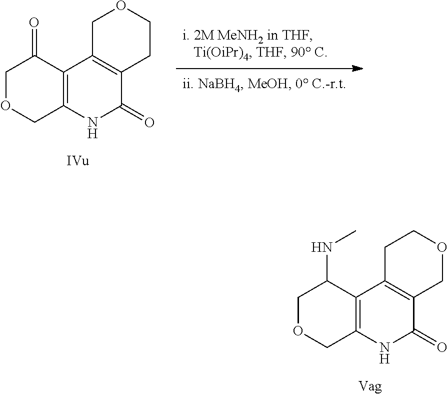 [SMILES: O=C1COCc2[nH]c(=O)c3c(c21)COCC3 ; CNC1COCc2[nH]c(=O)c3c(c21)CCOC3]

1-(Methylamino)-1,2,5,7,9,10-hexahydrodipyrano[3,4-b:4′,3′-d]pyridin-6(4H)-one was synthesized in an analogous manner as described above, from 5,7,9,10-tetrahydrodipyrano[3,4-b:4′,3′-d]pyridine-1,6(2H,4H)-dione (IVu) and methylamine. LCMS: m/z found 237.13 [M+H]⁺.

### 3-(3-Chloro-4-fluorophenyl)-1-methyl-1-(6-oxo-1,2,4,5,6,7,9,10-octahydrodipyrano[3,4-b:4′,3′-d]pyridin-1-yl)urea (Compounds 91 and 92)

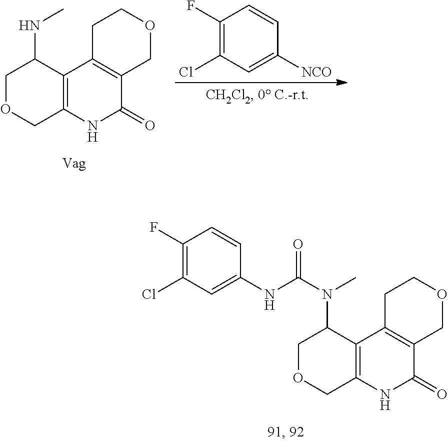

Racemic 3-(3-chloro-4-fluorophenyl)-1-methyl-1-(6-oxo-1,2,4,5,6,7,9,10-octahydrodipyrano[3,4-b:4′,3′-d]pyridin-1-yl)urea was synthesized in an analogous manner as described above, from 1-(methylamino)-1,2,5,7,9,10-hexahydrodipyrano[3,4-b:4′,3′-d]pyridin-6(4H)-one (Vag) and 2-chloro-1-fluoro-4-isocyanatobenzene. The enantiomers were subsequently separated by preparative SFC: Method isocratic, Mobile phase MeOH: CO₂— 60:40. Column: Chiralpak IG (30×250 mm), 5μ, flow rate: 100 g/min.

Enantiomer I (Compound 91): LCMS: m/z found 408.2/410.2 [M+H]⁺, RT=2.95 min, (Method A); ¹H NMR (400 MHz, DMSO-d₆): δ 11.51 (br s, 1H), 8.51 (s, 1H), 7.82-7.80 (m, 1H), 7.48-7.44 (m, 1H), 7.29 (t, 1H), 5.08-5.06 (m, 1H), 4.50 (d, 1H), 4.37-4.28 (m, 3H), 3.96 (d, 1H), 3.88-3.83 (m, 2H), 3.67-3.62 (m, 1H), 2.78 (s, 3H), 2.49-2.39 (m, 1H), 2.33-2.28 (m, 1H); Chiral analytical SFC: RT=1.79 min, Column: Chiralpak IG-3 (4.6×150 mm) 3 μm, 40% methanol, Flow rate: 3 g/min.

Enantiomer II (Compound 92): LCMS: m/z found 408.2/410.3 [M+H]⁺, RT=2.95 min, (Method A); ¹H NMR (400 MHz, DMSO-d₆): δ 11.51 (br s, 1H), 8.51 (s, 1H), 7.82-7.80 (m, 1H), 7.48-7.44 (m, 1H), 7.29 (t, 1H), 5.08-5.06 (m, 1H), 4.50 (d, 1H), 4.37-4.28 (m, 3H), 3.96 (d, 1H), 3.88-3.83 (m, 2H), 3.67-3.62 (m, 1H), 2.78 (s, 3H), 2.49-2.39 (m, 1H), 2.33-2.28 (m, 1H); Chiral analytical SFC: RT=4.90 min, Column: Chiralpak IG-3 (4.6×150 mm) 3 μm, 40% methanol, Flow rate: 3 g/min.

### 3-(3,4-Difluorophenyl)-1-methyl-1-(6-oxo-1,2,4,5,6,7,9,10-octahydrodipyrano[3,4-b:4′,3′-d]pyridin-1-yl)urea (Compounds 93 and 94)

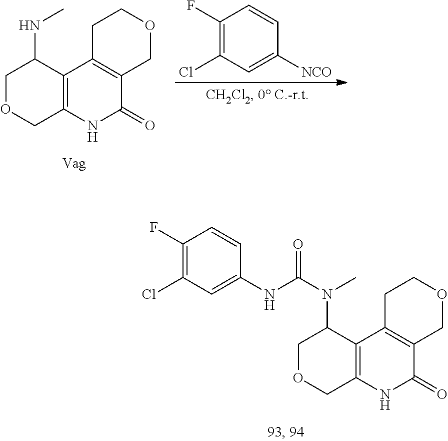

Racemic 3-(3,4-difluorophenyl)-1-methyl-1-(6-oxo-1,2,4,5,6,7,9,10-octahydrodipyrano[3,4-b:4′,3′-d]pyridin-1-yl)urea was synthesized in an analogous manner as described above, from 1-(methylamino)-1,2,5,7,9,10-hexahydrodipyrano[3,4-b:4′,3′-d]pyridin-6(4H)-one (Vag) and 1,2-difluoro-4-isocyanatobenzene. The enantiomers were subsequently separated by preparative SFC: Method isocratic, Mobile phase MeOH: CO₂ —30:70. Column: Chiralpak AD-H (30×250 mm), 5μ, flow rate: 90 g/min.

Enantiomer I (Compound 93): LCMS: m/z found 392.3 [M+H]⁺, RT=2.58 min, (Method A); ¹H NMR (400 MHz, DMSO-d₆): δ 11.51 (br s, 1H), 8.52 (br s, 1H), 7.71-7.65 (m, 1H), 7.34-7.28 (m, 2H), 5.08-5.06 (m, 1H), 4.50 (d, 1H), 4.41-4.27 (m, 3H), 3.96 (d, 1H), 3.87-3.78 (m, 2H), 3.67-3.62 (m, 1H), 2.78 (s, 3H), 2.49-2.37 (m, 1H), 2.34-2.22 (m, 1H); Chiral analytical SFC: RT=1.61 min, Column: Chiralpak AD-3 (4.6×150 mm) 3 μm, 30% methanol, Flow rate: 3 g/min.

Enantiomer II (Compound 94): LCMS: m/z found 392.3 [M+H]⁺, RT=2.58 min, (Method A); ¹H NMR (400 MHz, DMSO-d₆): δ 11.51 (br s, 1H), 8.52 (br s, 1H), 7.71-7.65 (m, 1H), 7.34-7.28 (m, 2H), 5.08-5.06 (m, 1H), 4.50 (d, 1H), 4.41-4.27 (m, 3H), 3.96 (d, 1H), 3.87-3.78 (m, 2H), 3.67-3.62 (m, 1H), 2.78 (s, 3H), 2.49-2.37 (m, 1H), 2.34-2.22 (m, 1H); Chiral analytical SFC: RT=3.85 min, Column: Chiralpak AD-3 (4.6×150 mm) 3 μm, 30% methanol, Flow rate: 3 g/min.

### 9-Fluoro-4,5-dihydropyrano[3,4-c]isoquinoline-1,6-dione (IVv)

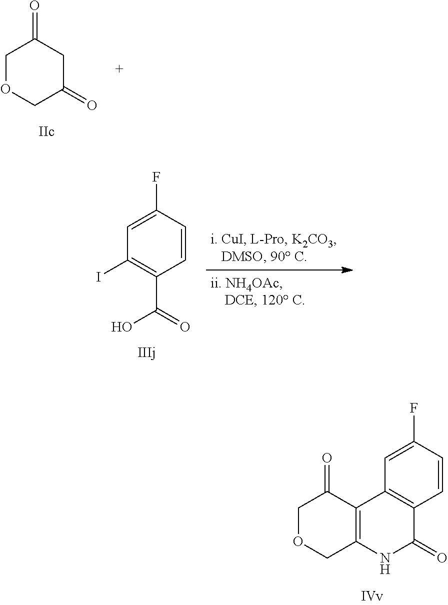 [SMILES: O=C1COCC(=O)C1 ; O=C(O)c1ccc(F)cc1I ; O=C1COCc2[nH]c(=O)c3ccc(F)cc3c21]

9-Fluoro-4,5-dihydropyrano[3,4-c]isoquinoline-1,6-dione was synthesized in an analogous manner as described above for IVh, from 4-fluoro-2-iodo-benzoic acid (IIIj) and tetrahydropyran-3,5-dione (IIc). LCMS m/z found 234; RT=0.69 min, (Method B); ¹H NMR (400 MHz, DMSO-d₆) δ 12.20 (s, 1H), 8.72 (dd, 1H), 8.28 (dd, 1H), 7.42 (td, 1H), 4.79 (s, 2H), 4.27 (s, 2H).

### 9-Fluoro-1-(methylamino)-1,2,4,5-tetrahydropyrano[3,4-c]isoquinolin-6-one (Vah)

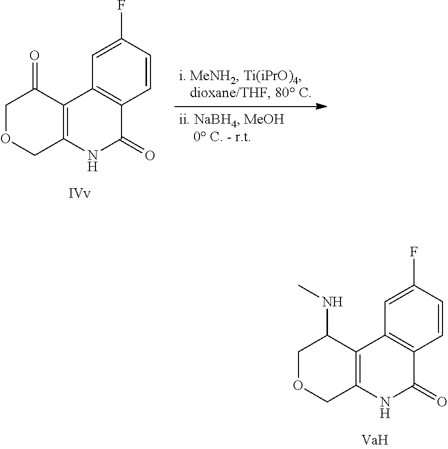 [SMILES: O=C1COCc2[nH]c(=O)c3ccc(F)cc3c21 ; CNC1COCc2[nH]c(=O)c3ccc(F)cc3c21]

9-Fluoro-1-(methylamino)-1,2,4,5-tetrahydropyrano[3,4-c]isoquinolin-6-one was synthesized in an analogous manner as described above, from 9-fluoro-4,5-dihydropyrano[3,4-c]isoquinoline-1,6-dione (IVv) and methylamine. ¹H NMR (400 MHz, CDCl₃) δ 11.78 (s, 1H), 8.41 (dd, 1H), 7.36 (dd, 1H), 7.17 (td, 1H), 4.70 (d, 1H), 4.62-4.53 (m, 1H), 4.42 (dd, 1H), 3.62 (dd, 1H), 3.50 (d, 1H), 2.61 (s, 3H).

### 3-(3-Chloro-4-fluorophenyl)-1-(9-fluoro-6-oxo-1,4,5,6-tetrahydro-2H-pyrano[3,4-c]isoquinolin-1-yl)-1-methylurea (Compound 73)

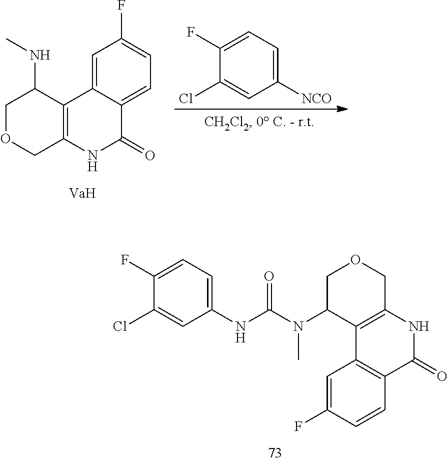

3-(3-Chloro-4-fluorophenyl)-1-(9-fluoro-6-oxo-1,4,5,6-tetrahydro-2H-pyrano[3,4-c]isoquinolin-1-yl)-1-methylurea was synthesized in an analogous manner as described above, from 9-fluoro-1-(methylamino)-1,2,4,5-tetrahydropyrano[3,4-c]isoquinolin-6-one (Vah) and 2-chloro-1-fluoro-4-isocyanatobenzene. LCMS m/z found 420.2 [M+H]⁺; RT=3.24 min (Method C, Shimadzu); ¹H NMR (400 MHz, DMSO-d₆) δ 11.53 (s, 1H), 8.59 (s, 1H), 8.28 (dd, 1H), 7.84 (dd, 1H), 7.51 (ddd, 1H), 7.39-7.29 (m, 2H), 7.23 (dd, 1H), 5.41 (s, 1H), 4.59 (d, 1H), 4.43 (dd, 1H), 4.10-4.02 (m, 1H), 3.94 (dd, 1H), 2.82 (s, 3H).

### 1-(Ethylamino)-9-fluoro-1,2,4,5-tetrahydropyrano[3,4-c]isoquinolin-6-one (Vai)

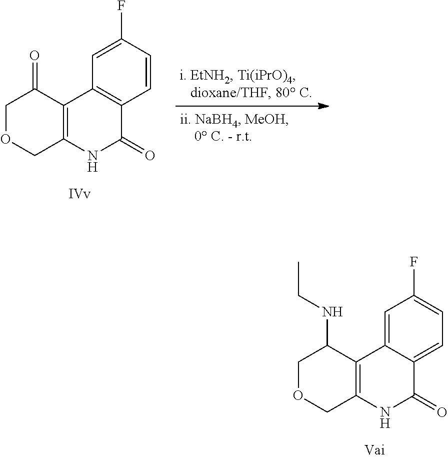 [SMILES: O=C1COCc2[nH]c(=O)c3ccc(F)cc3c21 ; CCNC1COCc2[nH]c(=O)c3ccc(F)cc3c21]

1-(Ethylamino)-9-fluoro-1,2,4,5-tetrahydropyrano[3,4-c]isoquinolin-6-one was synthesized in an analogous manner as described above, from 9-fluoro-4,5-dihydropyrano[3,4-c]isoquinoline-1,6-dione (IVv) and ethylamine. ¹H NMR (400 MHz, CDCl₃) δ 11.91 (s, 1H), 8.39 (dd, 1H), 7.38 (dd, 1H), 7.21-7.07 (m, 1H), 4.71 (d, 1H), 4.57 (d1H), 4.42-4.34 (m, 1H), 3.63 (m, 2H), 2.97 (dq, 1H), 2.78 (dq, 1H), 1.20 (t, 3H).

### 3-(3-Chloro-4-fluorophenyl)-1-ethyl-1-(9-fluoro-6-oxo-1,4,5,6-tetrahydro-2H-pyrano[3,4-c]isoquinolin-1-yl)urea (Compound 74)

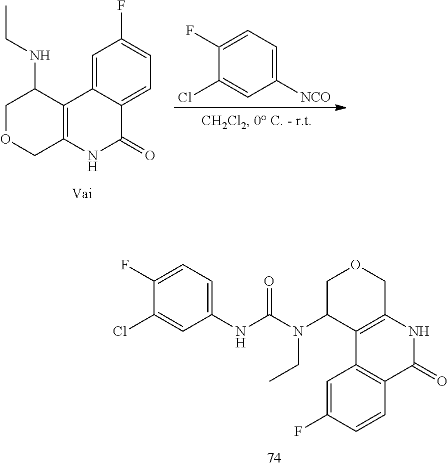

3-(3-Chloro-4-fluorophenyl)-1-ethyl-1-(9-fluoro-6-oxo-1,4,5,6-tetrahydro-2H-pyrano[3,4-c]isoquinolin-1-yl)urea was synthesized in an analogous manner as described above, from 1-(ethylamino)-9-fluoro-1,2,4,5-tetrahydropyrano[3,4-c]isoquinolin-6-one (Vai) and 2-chloro-1-fluoro-4-isocyanatobenzene. LCMS m/z found 434.2 [M+H]⁺; RT=3.33 min (Method C, Shimadzu); ¹H NMR (400 MHz, DMSO-d₆) δ 11.53 (s, 1H), 8.50 (s, 1H), 8.32-8.23 (m, 1H), 7.84 (ddd, 1H), 7.53 (dddd, 1H), 7.34 (ddt, 2H), 7.21 (dd, 1H), 5.42 (s, 1H), 4.60 (d, 1H), 4.49-4.40 (m, 1H), 4.04 (d, 1H), 3.92 (dd, 1H), 3.44 (dt, 1H), 3.33-3.22 (m, 1H), 0.85 (t, 3H).

### 3-(4-Fluoro-3-methylphenyl)-1-(9-fluoro-6-oxo-1,4,5,6-tetrahydro-2H-pyrano[3,4-c]isoquinolin-1-yl)-1-methylurea (Compound 75)

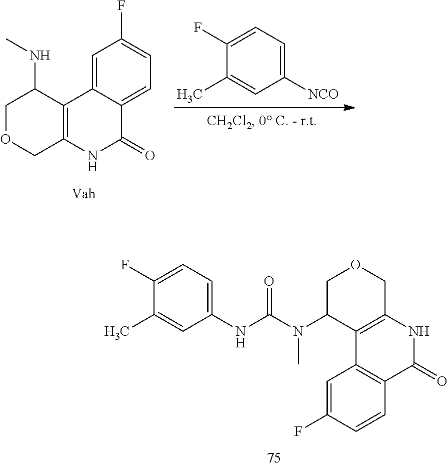

3-(4-Fluoro-3-methylphenyl)-1-(9-fluoro-6-oxo-1,4,5,6-tetrahydro-2H-pyrano[3,4-c]isoquinolin-1-yl)-1-methylurea was synthesized in an analogous manner as described above, from 9-fluoro-1-(methylamino)-1,2,4,5-tetrahydropyrano[3,4-c]isoquinolin-6-one (Vah) and 1-fluoro-4-isocyanato-2-methyl-benzene. LCMS m/z found 400.2 [M+H]⁺; RT=3.15 min (Method C, Shimadzu); ¹H NMR (400 MHz, DMSO-d₆) δ 11.51 (s, 1H), 8.36 (s, 1H), 8.28 (dd, 1H), 7.44 (dd, 1H), 7.39-7.29 (m, 2H), 7.26 (dd, 1H), 7.04 (t, 1H), 5.42 (d, 1H), 4.59 (d, 1H), 4.42 (dd, 1H), 4.08-4.00 (m, 1H), 3.93 (dd, 1H), 2.80 (s, 3H), 2.21 (d, 3H).

### 1-Ethyl-3-(4-fluoro-3-methylphenyl)-1-(9-fluoro-6-oxo-1,4,5,6-tetrahydro-2H-pyrano[3,4-c]isoquinolin-1-yl)urea (Compound 76)

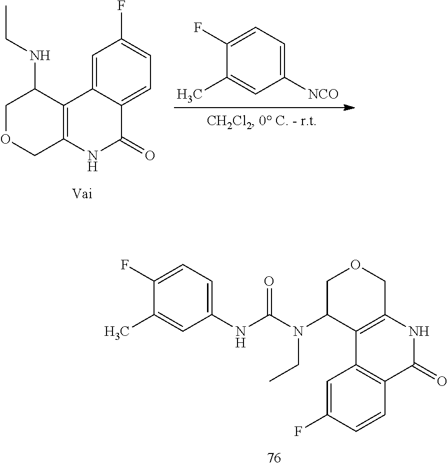

1-Ethyl-3-(4-fluoro-3-methylphenyl)-1-(9-fluoro-6-oxo-1,4,5,6-tetrahydro-2H-pyrano[3,4-c]isoquinolin-1-yl)urea was synthesized in an analogous manner as described above, from 1-(ethylamino)-9-fluoro-1,2,4,5-tetrahydropyrano[3,4-c]isoquinolin-6-one (Vai) and 1-fluoro-4-isocyanato-2-methyl-benzene. LCMS m/z found 414.2 [M+H]⁺; RT=3.21 min (Method C, Shimadzu); ¹H NMR (400 MHz, DMSO-d₆) δ 11.51 (s, 1H), 8.32-8.23 (m, 2H), 7.47-7.39 (m, 1H), 7.40-7.28 (m, 2H), 7.24 (dd, 1H), 7.05 (t, 1H), 5.42 (s, 1H), 4.59 (d, 1H), 4.48-4.39 (m, 1H), 4.02 (d, 1H), 3.92 (dd, 1H), 3.42 (dd, 1H), 3.33-3.13 (m, 1H), 2.22 (d, 3H), 0.85 (t, 3H).

### 3-(3-Cyano-4-fluorophenyl)-1-(9-fluoro-6-oxo-1,4,5,6-tetrahydro-2H-pyrano[3,4-c]isoquinolin-1-yl)-1-methylurea (Compound 77)

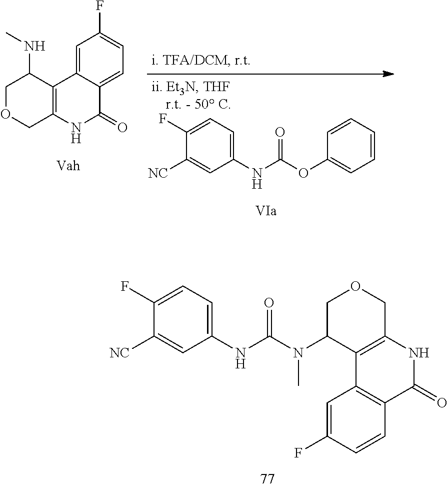 [SMILES: CNC1COCc2[nH]c(=O)c3ccc(F)cc3c21 ; N#Cc1cc(NC(=O)Oc2ccccc2)ccc1F ; CN(C(=O)Nc1ccc(F)c(C#N)c1)C1COCc2[nH]c(=O)c3ccc(F)cc3c21]

3-(3-Cyano-4-fluorophenyl)-1-(9-fluoro-6-oxo-1,4,5,6-tetrahydro-2H-pyrano[3,4-c]isoquinolin-1-yl)-1-methylurea was synthesized in an analogous manner as described above, from 9-fluoro-1-(methylamino)-1,2,4,5-tetrahydropyrano[3,4-c]isoquinolin-6-one (Vah) and phenyl N-(3-cyano-4-fluoro-phenyl)carbamate. LCMS m/z found 411.2 [M+H]⁺; RT=3.07 min (Method C, Shimadzu); ¹H NMR (400 MHz, DMSO-d6) δ 11.53 (s, 1H), 8.77 (s, 1H), 8.28 (dd, 1H), 8.05 (dd, 1H), 7.94-7.84 (m, 1H), 7.47 (t, 1H), 7.34 (td, 1H), 7.22 (dd, 1H), 5.40 (s, 1H), 4.59 (d, J=16.2 Hz, 1H), 4.43 (dd, 1H), 4.07 (d, 1H), 3.94 (dd, 1H), 2.83 (s, 3H).

### 3-(3-Cyano-4-fluorophenyl)-1-ethyl-1-(9-fluoro-6-oxo-1,4,5,6-tetrahydro-2H-pyrano[3,4-c]isoquinolin-1-yl)urea (Compound 78)

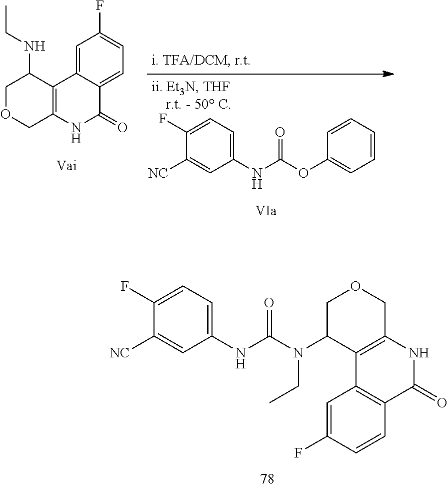 [SMILES: CCNC1COCc2[nH]c(=O)c3ccc(F)cc3c21 ; N#Cc1cc(NC(=O)Oc2ccccc2)ccc1F ; CCN(C(=O)Nc1ccc(F)c(C#N)c1)C1COCc2[nH]c(=O)c3ccc(F)cc3c21]

3-(3-Cyano-4-fluorophenyl)-1-ethyl-1-(9-fluoro-6-oxo-1,4,5,6-tetrahydro-2H-pyrano[3,4-c]isoquinolin-1-yl)urea was synthesized in an analogous manner as described above, from 1-(ethylamino)-9-fluoro-1,2,4,5-tetrahydropyrano[3,4-c]isoquinolin-6-one (Vai) and phenyl N-(3-cyano-4-fluoro-phenyl)carbamate. LCMS m/z found 425.2 [M+H]⁺; RT=3.17 min (Method C); ¹H NMR (400 MHz, DMSO-d₆) δ 11.53 (s, 1H), 8.66 (s, 1H), 8.28 (dd, 1H), 8.05 (dd, 1H), 7.96-7.87 (m, 1H), 7.48 (t, 1H), 7.34 (td, 1H), 7.19 (dd, 1H), 5.42 (d, 1H), 4.60 (d, 1H), 4.44 (dd, 1H), 4.05 (d, 1H), 3.93 (dd, 1H), 3.44 (dq, 1H), 3.29 (dt, 1H), 0.86 (t, 3H).

### 1,6-Dioxo-4,5-dihydropyrano[3,4-c]isoquinoline-8-carbonitrile (IVw)

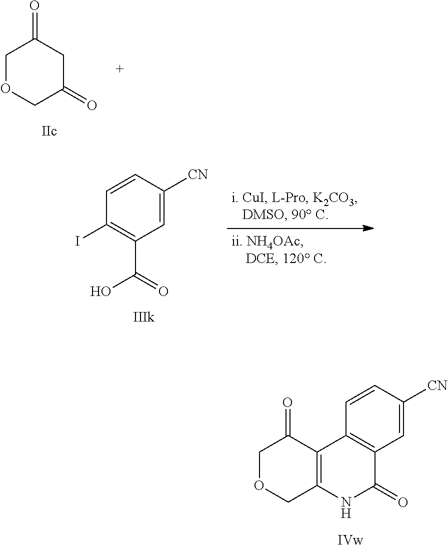 [SMILES: O=C1COCC(=O)C1 ; N#Cc1ccc(I)c(C(=O)O)c1 ; N#Cc1ccc2c3c([nH]c(=O)c2c1)COCC3=O]

1,6-Dioxo-4,5-dihydropyrano[3,4-c]isoquinoline-8-carbonitrile was synthesized in an analogous manner as described above, from 5-cyano-2-iodo-benzoic acid (IIIk) and tetrahydropyran-3,5-dione (IIc). LCMS m/z found 241.2 [M+H]⁺, RT=2.17 min (Method C); ¹H NMR (400 MHz, DMSO-d₆) δ 12.45 (s, 1H), 9.11 (d, 1H), 8.53 (d, 1H), 8.16 (dd, 1H), 4.80 (s, 2H), 4.29 (s, 2H).

### 1-(Methylamino)-6-oxo-1,2,4,5-tetrahydropyrano[3,4-c]isoquinoline-8-carbonitrile (Vaj)

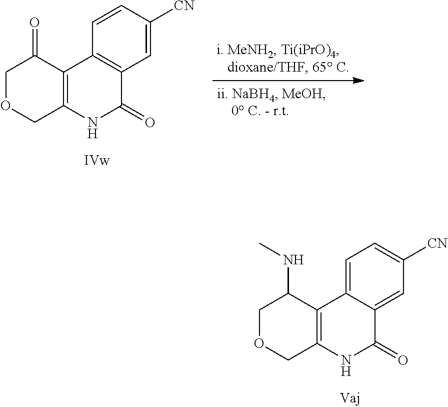 [SMILES: N#Cc1ccc2c3c([nH]c(=O)c2c1)COCC3=O ; CNC1COCc2[nH]c(=O)c3cc(C#N)ccc3c21]

1-(Methylamino)-6-oxo-1,2,4,5-tetrahydropyrano[3,4-c]isoquinoline-8-carbonitrile was synthesized in an analogous manner as described above, from 1,6-dioxo-4,5-dihydropyrano[3,4-c]isoquinoline-8-carbonitrile (IVw) and methanamine. LCMS m/z found 256.2 [M+H]⁺, RT=0.44 min (Method B); ¹H NMR (400 MHz, Methanol-d₄) δ 8.63-8.58 (m, 1H), 7.93 (dd, 1H), 7.84 (d, 1H), 4.59 (d, 1H), 4.48 (dd, 1H), 4.42-4.33 (m, 1H), 3.70-3.55 (m, 2H), 2.55 (s, 3H).

### 1-(Ethylamino)-6-oxo-1,2,4,5-tetrahydropyrano[3,4-c]isoquinoline-8-carbonitrile (Vak)

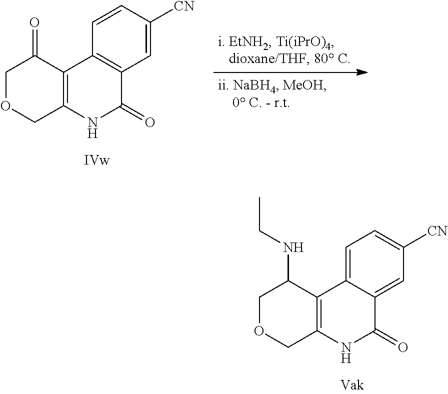 [SMILES: N#Cc1ccc2c3c([nH]c(=O)c2c1)COCC3=O ; CCNC1COCc2[nH]c(=O)c3cc(C#N)ccc3c21]

1-(Ethylamino)-6-oxo-1,2,4,5-tetrahydropyrano[3,4-c]isoquinoline-8-carbonitrile was synthesized in an analogous manner as described above, from 1,6-dioxo-4,5-dihydropyrano[3,4-c]isoquinoline-8-carbonitrile (IVw) and ethylamine. LCMS m/z found 270.2 [M+H]⁺, RT=0.45 min (Method B); ¹H NMR (400 MHz, DMSO-d₆) δ 11.57 (s, 1H), 8.49 (dt, 1H), 8.08 (ddd, 1H), 8.00-7.92 (m, 1H), 4.46 (d, 1H), 4.39 (d, 1H), 4.22 (d, 1H), 3.72 (s, 1H), 3.57 (dd, 1H), 2.79 (dq, 1H), 2.74-2.61 (m, 1H), 1.04 (td, 3H).

### 3-(3-Chloro-4-fluorophenyl)-1-(8-cyano-6-oxo-1,4,5,6-tetrahydro-2H-pyrano[3,4-c]isoquinolin-1-yl)-1-methylurea (Compound 89)

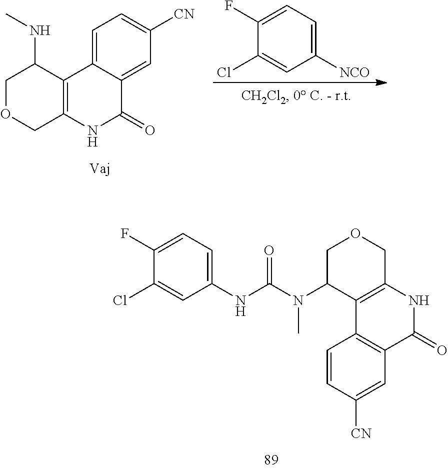

3-(3-Chloro-4-fluorophenyl)-1-(8-cyano-6-oxo-1,4,5,6-tetrahydro-2H-pyrano[3,4-c]isoquinolin-1-yl)-1-methylurea was synthesized in an analogous manner as described above, from 1-(methylamino)-6-oxo-1,2,4,5-tetrahydropyrano[3,4-c]isoquinoline-8-carbonitrile (Vaj) and 2-chloro-1-fluoro-4-isocyanatobenzene. LCMS m/z found 427.25 [M+H]⁺; RT=3.25 min (Method C); ¹H NMR (400 MHz, DMSO-d₆) δ 11.84 (s, 1H), 8.61-8.52 (m, 2H), 8.14 (ddd, 1H), 7.88 (ddd, 1H), 7.67-7.59 (m, 1H), 7.52 (dddd, 1H), 7.33 (td, 1H), 5.46 (d, 1H), 4.62 (d, 1H), 4.46 (dd, 1H), 4.07 (d, 1H), 3.94 (dd, 1H), 2.80 (s, 3H).

### 3-(3-Chloro-4-fluorophenyl)-1-(8-cyano-6-oxo-1,4,5,6-tetrahydro-2H-pyrano[3,4-c]isoquinolin-1-yl)-1-ethylurea (Compound 90)

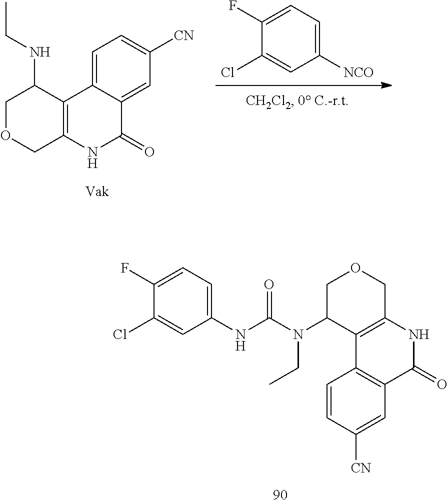

3-(3-Chloro-4-fluorophenyl)-1-(8-cyano-6-oxo-1,4,5,6-tetrahydro-2H-pyrano[3,4-c]isoquinolin-1-yl)-1-ethylurea was synthesized in an analogous manner as described above, from 1-(ethylamino)-6-oxo-1,2,4,5-tetrahydropyrano[3,4-c]isoquinoline-8-carbonitrile (Vak) and 2-chloro-1-fluoro-4-isocyanatobenzene. LCMS m/z found 441.25 [M+H]⁺; RT=3.33 min (Method C); ¹H NMR (400 MHz, DMSO-d₆) δ 11.84 (s, 1H), 8.55 (dd, 1H), 8.48 (s, 1H), 8.14 (dd, 1H), 7.88 (dd, 1H), 7.64-7.57 (m, 1H), 7.54 (ddd, 1H), 7.33 (t, 1H), 5.47 (s, 1H), 4.63 (d, 1H), 4.47 (dd, 1H), 4.04 (d, 1H), 3.92 (dd, 1H), 3.47-3.35 (m, 1H), 3.33-3.18 (m, 1H), 0.84 (t, 3H).

### 2H-Pyrano[3,4-b]thieno[3,2-d]pyridine-1,6(4H,5H)-dione (IVx)

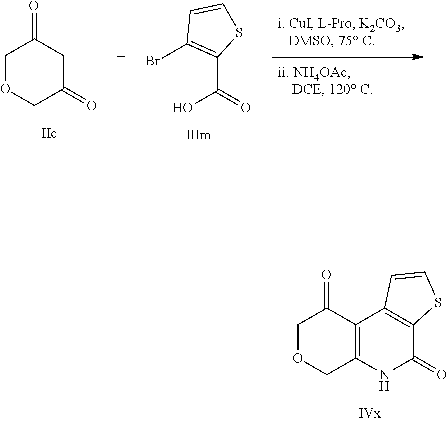 [SMILES: O=C1COCC(=O)C1 ; O=C(O)c1sccc1Br ; O=C1COCc2[nH]c(=O)c3sccc3c21]

2H-Pyrano[3,4-b]thieno[3,2-d]pyridine-1,6(4H,5H)-dione was synthesized in an analogous manner as described above, from 3-bromothiophene-2-carboxylic acid (IIIm) and tetrahydropyran-3,5-dione (IIc). LCMS m/z found 222.1 [M+H]⁺; RT=0.59 min (Method B); ¹H NMR (400 MHz, DMSO-d₆) δ 12.31 (s, 1H), 8.21 (d, 1H), 8.11 (d, 1H), 4.81 (s, 2H), 4.28 (s, 2H).

### 1-(Methylamino)-1,5-dihydro-2H-pyrano[3,4-b]thieno[3,2-d]pyridin-6(4H)-one (Val)

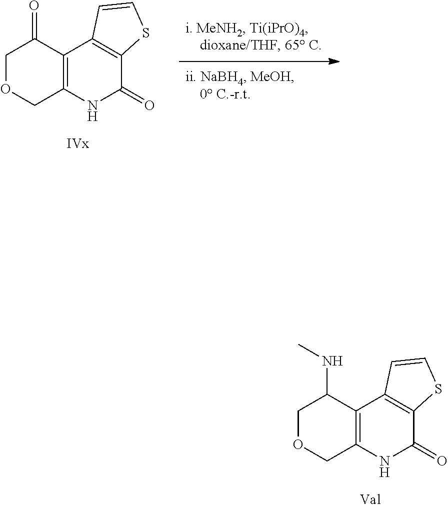 [SMILES: O=C1COCc2[nH]c(=O)c3sccc3c21 ; CNC1COCc2[nH]c(=O)c3sccc3c21]

1-(Methylamino)-1,5-dihydro-2H-pyrano[3,4-b]thieno[3,2-d]pyridin-6(4H)-one was synthesized in an analogous manner as described above, from 2H-pyrano[3,4-b]thieno[3,2-d]pyridine-1,6(4H,5H)-dione (IVx) and methanamine. LCMS m/z found 237.1 [M+H]⁺; RT=0.36 min (Method B); ¹H NMR (400 MHz, CDCl₃) δ 7.78 (d, 1H), 7.38 (d, 1H), 4.78 (d, 1H), 4.61 (d, 1H), 4.35 (d, 1H), 3.70-3.57 (m, 2H), 2.58 (s, 3H).

### 3-(3-Chloro-4-fluorophenyl)-1-methyl-1-(6-oxo-1,4,5,6-tetrahydro-2H-pyrano[3,4-b]thieno[3,2-d]pyridin-1-yl)urea (Compound 95)

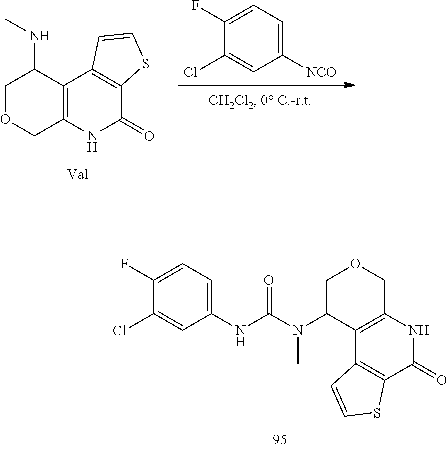

3-(3-Chloro-4-fluorophenyl)-1-methyl-1-(6-oxo-1,4,5,6-tetrahydro-2H-pyrano[3,4-b]thieno[3,2-d]pyridin-1-yl)urea was synthesized in an analogous manner as described above, from 1-(methylamino)-1,5-dihydro-2H-pyrano[3,4-b]thieno[3,2-d]pyridin-6(4H)-one (Val) and 2-chloro-1-fluoro-4-isocyanatobenzene. LCMS m/z found 408.2/410.2 [M+H]⁺; RT=3.46 min (Method A); ¹H NMR (400 MHz, DMSO-d₆) δ 11.63 (s, 1H), 8.58 (s, 1H), 8.08 (dd, 1H), 7.87 (dd, 1H), 7.52 (dddd, 1H), 7.32 (t, 1H), 7.17 (dd, 1H), 5.47 (s, 1H), 4.61 (d, 1H), 4.43 (dd, 1H), 4.02 (dd, 1H), 3.94 (dd, 1H), 2.80 (s, 3H).

5H-pyrano[3,4-b]thieno[2,3-d]pyridine-4,9(6H,8H)-dione (IVy)

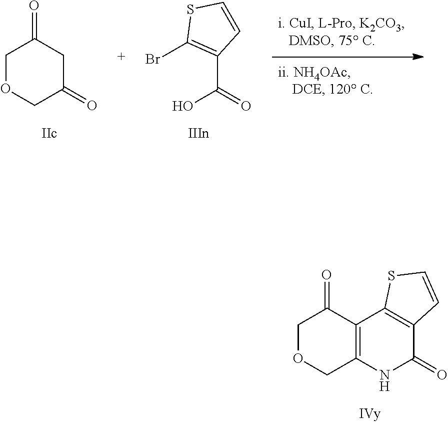

5H-Pyrano[3,4-b]thieno[2,3-d]pyridine-4,9(6H,8H)-dione was synthesized in an analogous manner as described above, from 2-bromothiophene-3-carboxylic acid (IIIn) and tetrahydropyran-3,5-dione (IIc). LCMS m/z found 222.1 [M+H]⁺; RT=0.58 min (Method B); ¹H NMR (400 MHz, DMSO-d₆) δ 12.28 (s, 1H), 7.71 (d, 1H), 7.51 (d, 1H), 4.83 (s, 2H), 4.35 (s, 2H).

### 9-(Methylamino)-8,9-dihydro-5H-pyrano[3,4-b]thieno[2,3-d]pyridin-4(6H)-one (Vam)

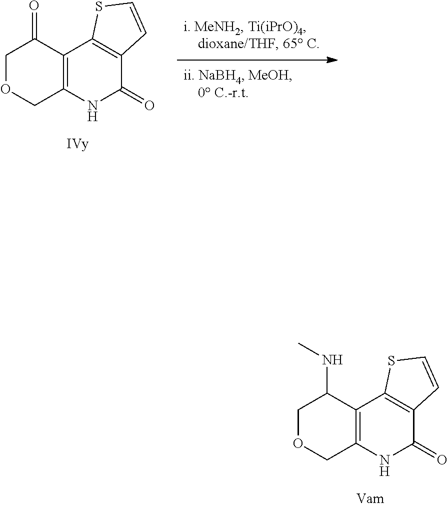 [SMILES: O=C1COCc2[nH]c(=O)c3ccsc3c21 ; CNC1COCc2[nH]c(=O)c3ccsc3c21]

9-(Methylamino)-8,9-dihydro-5H-pyrano[3,4-b]thieno[2,3-d]pyridin-4(6H)-one was synthesized in an analogous manner as described above, from 5H-pyrano[3,4-b]thieno[2,3-d]pyridine-4,9(6H,8H)-dione (IVy) and methylamine. LCMS m/z found 237.1 [M+H]⁺; RT=0.36 min (Method B); ¹H NMR (400 MHz, CDCl₃) δ 7.61 (d, 1H), 7.30 (d, 1H), 4.74 (d, 1H), 4.63-4.57 (m, 1H), 4.25 (dd, 1H), 3.82-3.74 (m, 1H), 3.60 (s, 1H), 2.59 (s, 3H).

### 3-(3-chloro-4-fluorophenyl)-1-methyl-1-(4-oxo-4,6,8,9-tetrahydro-5H-pyrano[3,4-b]thieno[2,3-d]pyridin-9-yl)urea (Compound 96)

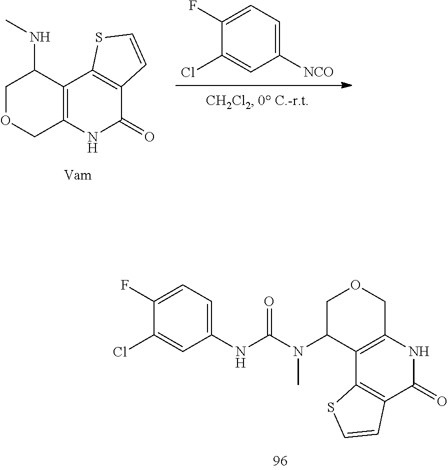

3-(3-Chloro-4-fluorophenyl)-1-methyl-1-(4-oxo-4,6,8,9-tetrahydro-5H-pyrano[3,4-b]thieno[2,3-d]pyridin-9-yl)urea was synthesized in an analogous manner as described above, from 9-(methylamino)-8,9-dihydro-5H-pyrano[3,4-b]thieno[2,3-d]pyridin-4(6H)-one (Vam) and 2-chloro-1-fluoro-4-isocyanatobenzene. LCMS m/z found 408.2/410.2 [M+H]⁺; RT=3.37 min (Method A); ¹H NMR (400 MHz, DMSO-d₆) δ 11.56 (s, 1H), 8.64 (s, 1H), 7.86 (ddd, 1H), 7.58-7.55 (m, 1H), 7.55-7.49 (m, 1H), 7.47 (dd, 1H), 7.33 (td, 1H), 5.36 (s, 1H), 4.60 (d, 1H), 4.44 (d, 1H), 4.04-3.90 (m, 2H), 2.77 (s, 3H).

6H-Pyrano[3,4-b]thieno[3,4-d]pyridine-4,9(5H,8H)-dione (IVz)

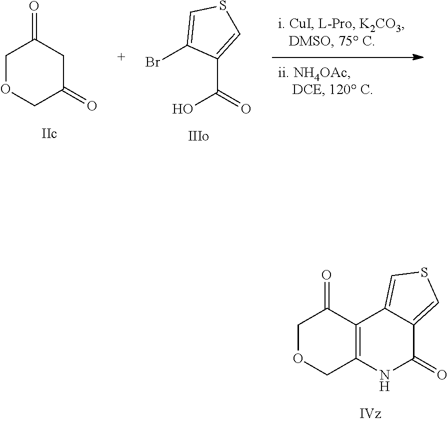

6H-Pyrano[3,4-b]thieno[3,4-d]pyridine-4,9(5H,8H)-dione was synthesized in an analogous manner as described above, from 4-bromothiophene-3-carboxylic acid (IIIo) and tetrahydropyran-3,5-dione (IIc). LCMS m/z found 222.1 [M+H]⁺; RT=0.58 min (Method B); ¹H NMR (400 MHz, DMSO-d₆) δ 12.28 (s, 1H), 7.71 (d, 1H), 7.51 (d, 1H), 4.83 (s, 2H), 4.35 (s, 2H).

### 9-(Methylamino)-8,9-dihydro-6H-pyrano[3,4-b]thieno[3,4-d]pyridin-4(5H)-one (Van)

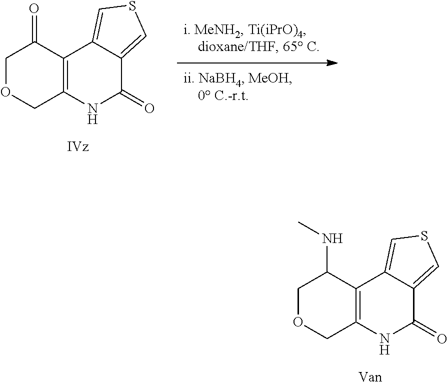 [SMILES: O=C1COCc2[nH]c(=O)c3cscc3c21 ; CNC1COCc2[nH]c(=O)c3cscc3c21]

9-(Methylamino)-8,9-dihydro-6H-pyrano[3,4-b]thieno[3,4-d]pyridin-4(5H)-one was synthesized in an analogous manner as described above, from 6H-pyrano[3,4-b]thieno[3,4-d]pyridine-4,9(5H,8H)-dione (IVz) and methylamine. LCMS m/z found 237.1 [M+H]⁺; RT=0.40 min (Method B); ¹H NMR (400 MHz, CDCl₃) δ 10.72 (s, 1H), 8.37 (dd, 1H), 7.45-7.38 (m, 1H), 4.58 (d, 1H), 4.47 (dd, 1H), 4.29 (dd, 1H), 3.64 (dd, 1H), 3.52 (p, 1H), 2.58 (s, 3H).

### 3-(3-Chloro-4-fluorophenyl)-1-methyl-1-(4-oxo-4,5,8,9-tetrahydro-6H-pyrano[3,4-b]thieno[3,4-d]pyridin-9-yl)urea (Compound 105)

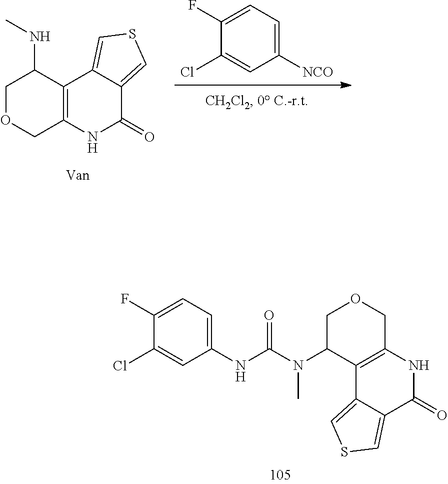

3-(3-Chloro-4-fluorophenyl)-1-methyl-1-(4-oxo-4,5,8,9-tetrahydro-6H-pyrano[3,4-b]thieno[3,4-d]pyridin-9-yl)urea was synthesized in an analogous manner as described above, from 9-(methylamino)-8,9-dihydro-6H-pyrano[3,4-b]thieno[3,4-d]pyridin-4(5H)-one (Van) and 2-chloro-1-fluoro-4-isocyanatobenzene. LCMS m/z found 408.2/410.2 [M+H]⁺; RT=3.64 min (Method A); ¹H NMR (400 MHz, DMSO-d₆) δ 10.78 (s, 1H), 8.56 (s, 1H), 8.46 (dd, 1H), 7.88 (dd, 1H), 7.52 (ddd, 1H), 7.40-7.27 (m, 2H), 5.36 (s, 1H), 4.47 (d, 1H), 4.32 (dd, 1H), 4.02-3.87 (m, 2H), 2.83 (s, 3H).

### 5-(Isobutylamino)-4-(trifluoromethyl)-5,6,7,8-tetrahydro-1H-quinolin-2-one (Vao)

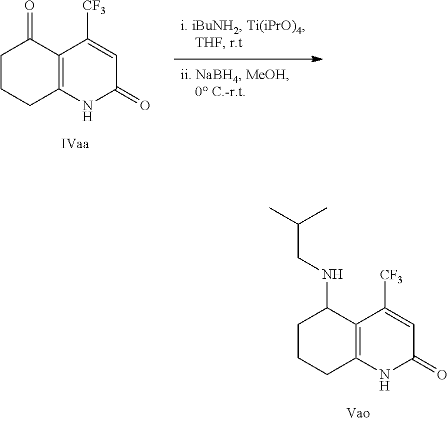 [SMILES: O=C1CCCc2[nH]c(=O)cc(C(F)(F)F)c21 ; CC(C)CNC1CCCc2[nH]c(=O)cc(C(F)(F)F)c21]

Tetraisopropoxytitanium (0.25 g, 0.87 mmol) was added to a mixture of 4-(trifluoromethyl)-1,6,7,8-tetrahydroquinoline-2,5-dione (IVaa, 50 mg, 0.22 mmol) and 2-methylpropan-1-amine, (65 uL, 0.65 mmol) in THF (2 mL) under a nitrogen atmosphere and the mixture was stirred at room temperature for 16 hours. The reaction mixture was diluted with 3 mL anhydrous methanol and cooled in an ice bath. Sodium borohydride (16 mg, 0.43 mmol) was added in one portion and the reaction mixture was stirred for 5 minutes, and the ice bath was removed. After an additional 1 h, the reaction was quenched by addition of brine (1 mL), diluted with 20 mL of EtOAc, and stirred for 15 min. The mixture was filtered through CELITE®, and the filter cake was washed with an additional 15 mL of EtOAc. The combined filtrate was dried over sodium sulfate, filtered and the solvent evaporated. The material was used in the next step without further purification: 5-(isobutylamino)-4-(trifluoromethyl)-5,6,7,8-tetrahydro-1H-quinolin-2-one (40.0 mg, 64.1%). ¹H NMR (400 MHz, Methanol-d₄) δ 6.66 (s, 1H), 3.77 (s, 1H), 2.82-2.57 (m, 2H), 2.51 (dd, 1H), 2.35 (dd, 1H), 2.15 (dd, 2H), 1.66 (hept, 2H), 1.46 (t, 1H), 0.92 (q, 6H).

### 3-(3-Chloro-4-fluorophenyl)-1-isobutyl-1-(2-oxo-4-(trifluoromethyl)-1,2,5,6,7,8-hexahydroquinolin-5-yl)urea (Compound 1)

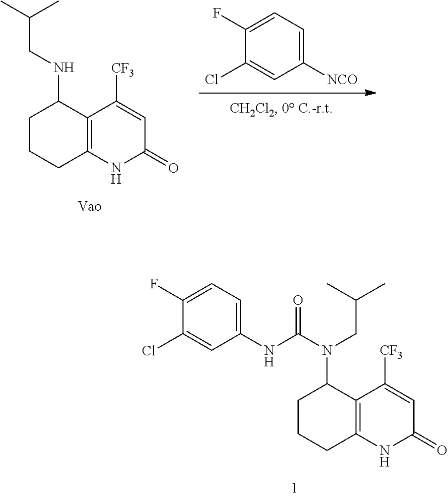

3-(3-Chloro-4-fluorophenyl)-1-isobutyl-1-(2-oxo-4-(trifluoromethyl)-1,2,5,6,7,8-hexahydroquinolin-5-yl)urea was synthesized in an analogous manner as described above, from 5-(isobutylamino)-4-(trifluoromethyl)-5,6,7,8-tetrahydro-1H-quinolin-2-one (Vao) and 2-chloro-1-fluoro-4-isocyanatobenzene. LCMS m/z found 460.1/462.1 [M+H]⁺; RT=4.70 min (Method A); ¹H NMR (400 MHz, Methanol-d₄) δ 7.50 (dd, 1H), 7.28-7.19 (m, 1H), 7.12 (t, 1H), 6.71 (s, 1H), 3.30 (p, 2H), 3.08 (d, 1H), 2.82 (dt, 1H), 2.75-2.64 (m, 1H), 2.12 (s, 1H), 2.06-1.98 (m, 3H), 1.81 (t, 2H), 0.88 (dd, 6H).

### 5-(Methylamino)-4-(trifluoromethyl)-5,6,7,8-tetrahydro-1H-quinolin-2-one (Vap)

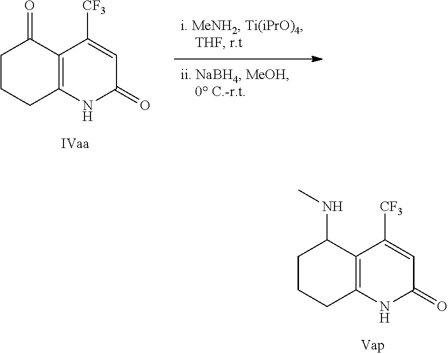 [SMILES: O=C1CCCc2[nH]c(=O)cc(C(F)(F)F)c21 ; CNC1CCCc2[nH]c(=O)cc(C(F)(F)F)c21]

5-(Methylamino)-4-(trifluoromethyl)-5,6,7,8-tetrahydro-1H-quinolin-2-one was synthesized in an analogous manner as described above, from 4-(trifluoromethyl)-1,6,7,8-tetrahydroquinoline-2,5-dione (IVaa) and methylamine. ¹H NMR (400 MHz, CDCl₃) δ 13.83 (s, 1H), 6.73 (s, 1H), 3.69 (d, 1H), 2.94-2.76 (m, 1H), 2.67 (ddd, 1H), 2.40 (d, 3H), 2.22-2.03 (m, 2H), 1.79-1.67 (m, 1H), 1.48-1.34 (m, 1H).

### 3-(3-Chloro-4-fluorophenyl)-1-methyl-1-(2-oxo-4-(trifluoromethyl)-1,2,5,6,7,8-hexahydroquinolin-5-yl)urea (Compound 2)

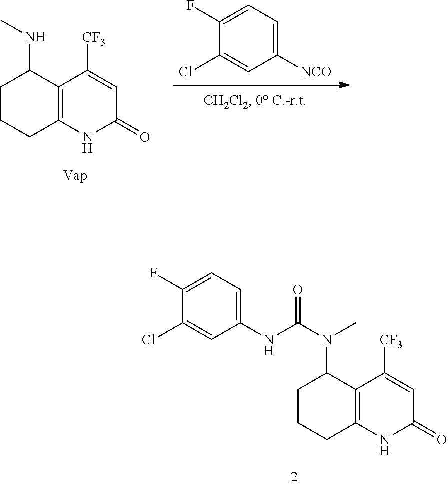

3-(3-Chloro-4-fluorophenyl)-1-methyl-1-(2-oxo-4-(trifluoromethyl)-1,2,5,6,7,8-hexahydroquinolin-5-yl)urea was synthesized in an analogous manner as described above, from 5-(methylamino)-4-(trifluoromethyl)-5,6,7,8-tetrahydro-1H-quinolin-2-one (Vap) and 2-chloro-1-fluoro-4-isocyanatobenzene. LCMS m/z found 418.1/420.2 [M+H]⁺; RT=3.87 min (Method A); ¹H NMR (400 MHz, Methanol-d₄) δ 7.60 (dd, 1H), 7.32 (ddd, 1H), 7.14 (t, 1H), 6.74 (d, 1H), 5.49 (s, 1H), 2.83-2.65 (m, 2H), 2.78 (s, 3H), 2.03 (dt, 1H), 1.89 (m, 3H).

### 5-(3-Hydroxypropylamino)-4-(trifluoromethyl)-5,6,7,8-tetrahydro-1H-quinolin-2-one (Vaq)

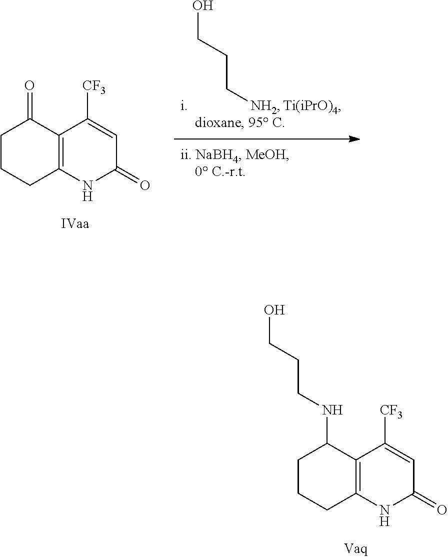 [SMILES: O=C1CCCc2[nH]c(=O)cc(C(F)(F)F)c21 ; NCCCO ; O=c1cc(C(F)(F)F)c2c([nH]1)CCCC2NCCCO]

5-(3-Hydroxypropylamino)-4-(trifluoromethyl)-5,6,7,8-tetrahydro-1H-quinolin-2-one was synthesized in an analogous manner as described above, from 4-(trifluoromethyl)-1,6,7,8-tetrahydroquinoline-2,5-dione (IVaa) and 3-aminopropan-1-ol. LCMS m/z found 291.2 [M+H]⁺; RT=0.49 min (Method B).

### 3-(3-Chloro-4-fluorophenyl)-1-(3-hydroxypropyl)-1-(2-oxo-4-(trifluoromethyl)-1,2,5,6,7,8-hexahydroquinolin-5-yl)urea (Compound 3)

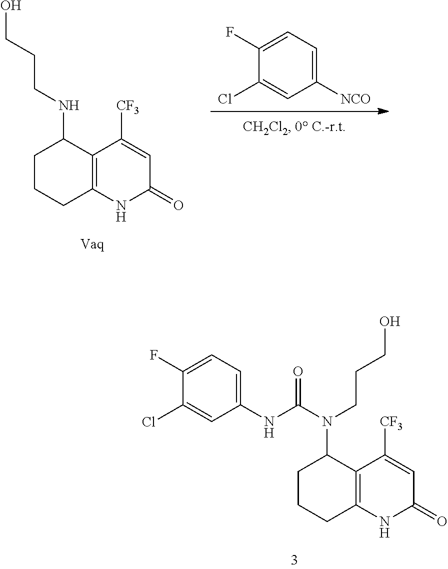

3-(3-Chloro-4-fluorophenyl)-1-(3-hydroxypropyl)-1-(2-oxo-4-(trifluoromethyl)-1,2,5,6,7,8-hexahydroquinolin-5-yl)urea was synthesized in an analogous manner as described above, from 5-(3-hydroxypropylamino)-4-(trifluoromethyl)-5,6,7,8-tetrahydro-1H-quinolin-2-one (Vaq) and 2-chloro-1-fluoro-4-isocyanatobenzene. LCMS m/z found 462.1/464.1 [M+H]⁺; RT=3.80 min (Method A); ¹H NMR (400 MHz, Methanol-d₄) δ 7.61 (dd, 1H), 7.31 (ddd, 1H), 7.13 (t, 1H), 6.73 (s, 1H), 5.48 (s, 1H), 3.52 (hept, 2H), 3.27 (d, 2H), 2.97-2.55 (m, 3H), 2.06 (td, 1H), 2.02-1.79 (m, 4H), 1.78-1.54 (m, 2H).

### 5-(Isobutylamino)-4-(trifluoromethyl)-1,5,6,7-tetrahydrocyclopenta[b]pyridin-2-one (Var)

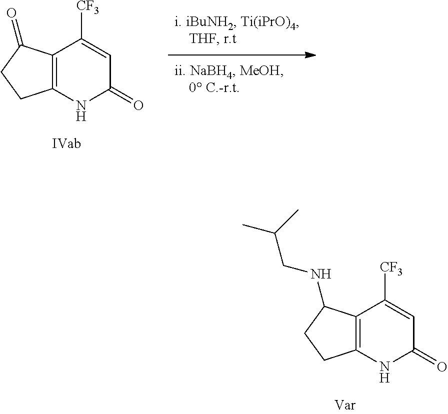 [SMILES: O=C1CCc2[nH]c(=O)cc(C(F)(F)F)c21 ; CC(C)CNC1CCc2[nH]c(=O)cc(C(F)(F)F)c21]

5-(Isobutylamino)-4-(trifluoromethyl)-1,5,6,7-tetrahydrocyclopenta[b]pyridin-2-one was synthesized in an analogous manner as described above, from 4-(trifluoromethyl)-6,7-dihydro-1H-cyclopenta[b]pyridine-2,5-dione (IVab) and 2-methylpropan-1-amine. LCMS m/z found 275.2 [M+H]⁺; RT=0.61 min (Method B); ¹H NMR (400 MHz, CDCl₃) δ 6.68 (d, 1H), 4.30 (d, 1H), 3.14 (dt, 1H), 2.85-2.73 (m, 1H), 2.39 (dd, 2H), 2.33 (dd, 1H), 2.31-2.16 (m, 1H), 2.17-2.01 (m, 1H), 1.67 (dq, 1H), 0.89 (d, 6H). 3-(3-Chloro-4-fluorophenyl)-1-isobutyl-1-(2-oxo-4-(trifluoromethyl)-2,5,6,7-tetrahydro-1H-cyclopenta[b]pyridin-5-yl)urea (Compound 4)

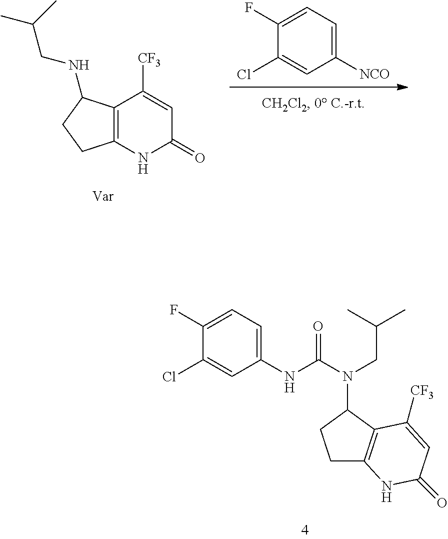

3-(3-Chloro-4-fluorophenyl)-1-isobutyl-1-(2-oxo-4-(trifluoromethyl)-2,5,6,7-tetrahydro-1H-cyclopenta[b]pyridin-5-yl)urea was synthesized in an analogous manner as described above, from 5-(isobutylamino)-4-(trifluoromethyl)-1,5,6,7-tetrahydrocyclopenta[b]pyridin-2-one (Var) and 2-chloro-1-fluoro-4-isocyanatobenzene. LCMS m/z found 446.1/448.1 [M+H]⁺; RT=4.64 min (Method A); ¹H NMR (400 MHz, Methanol-d₄) δ 7.45 (dd, 1H), 7.19 (d, 1H), 7.10 (t, 1H), 6.60 (s, 1H), 3.35 (s, 1H), 3.26-3.01 (m, 3H), 2.82 (ddd, 1H), 2.66 (dtd, 1H), 2.33 (s, 1H), 2.09-1.81 (m, 1H), 0.97 (dd, 6H).

### 3-(3,4-Difluorophenyl)-1-isobutyl-1-(2-oxo-4-(trifluoromethyl)-2,5,6,7-tetrahydro-1H-cyclopenta[b]pyridin-5-yl)urea (Compound 5)

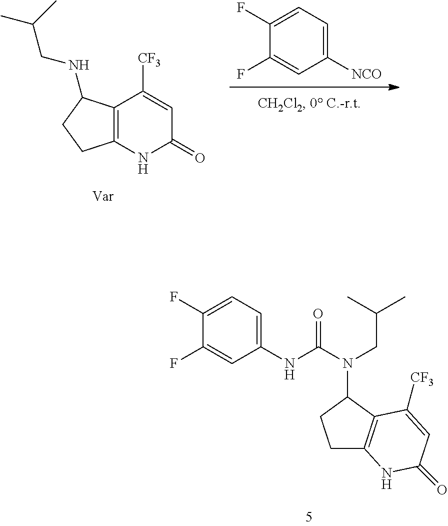

3-(3,4-Difluorophenyl)-1-isobutyl-1-(2-oxo-4-(trifluoromethyl)-2,5,6,7-tetrahydro-1H-cyclopenta[b]pyridin-5-yl)urea was synthesized in an analogous manner as described above, from 5-(isobutylamino)-4-(trifluoromethyl)-1,5,6,7-tetrahydrocyclopenta[b]pyridin-2-one (Var) and 1,2-difluoro-4-isocyanato-benzene. LCMS m/z found 430.2 [M+H]⁺; RT=4.37 min (Method A); ¹H NMR (400 MHz, Methanol-d₄) δ 7.29 (t, 1H), 7.11 (dt, 1H), 7.02 (s, 1H), 6.60 (s, 1H), 3.35 (s, 1H), 3.26-2.94 (m, 2H), 2.82 (ddd, 1H), 2.66 (dtd, 1H), 2.33 (s, 1H), 2.13-1.82 (m, 1H), 0.96 (dd, 6H).

### tert-Butyl-8-fluoro-1,6-dioxo-1,4,5,6-tetrahydrobenzo[c][1,7]naphthyridine-3(2H)-carboxylate (IVac)

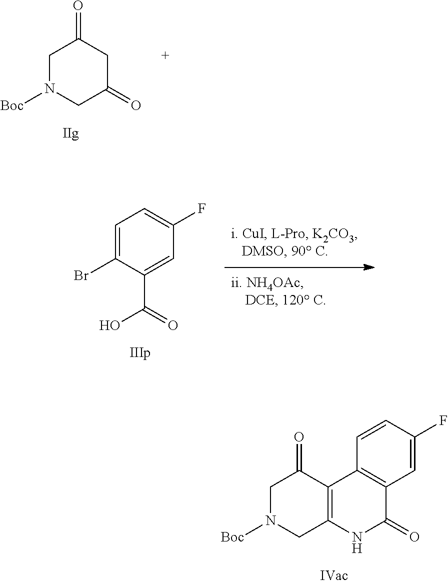 [SMILES: CC(C)(C)OC(=O)N1CC(=O)CC(=O)C1 ; O=C(O)c1cc(F)ccc1Br ; CC(C)(C)OC(=O)N1CC(=O)c2c([nH]c(=O)c3cc(F)ccc23)C1]

Step i: A mixture of 3.0 g (10.56 mmol, 1.0 eq) of 4-fluoro-2-bromobenzoic acid (IIIp), 2.7 g (12.68, 1.2 eq.) of tert-butyl 3,5-dioxopiperidine-1-carboxylate (IIg), 5.8 g (42.2 mmol, 4.0 eq.) of potassium carbonate, 0.25 g (2.11 mmol, 0.2 eq.) of L-proline and 0.2 g (1.05 mmol, 0.1 eq.) of copper(I)iodide in 15 mL of dry DMSO under a nitrogen atmosphere was stirred for 16 h at 110° C. (Note: Reaction was performed on 3×3 g scale in parallel). On cooling to room temperature, the triplicate reaction mixtures were combined and diluted with cold water (100 mL). The mixture was then acidified with saturated citric acid solution (100 mL). The resulting suspension was filtered, and the filtrate was extracted with ethyl acetate (3×200 mL). The combined organic layer was washed with brine (100 mL), dried over anhydrous sodium sulfate, filtered and concentrated under reduced pressure to afford 12.2 g of a mixture of tert-butyl 8-fluoro-1,6-dioxo-1,2,4,6-tetrahydro-3H-isochromeno[3,4-c]pyridine-3-carboxylate and 2-(1-(tert-butoxycarbonyl)-5-hydroxy-3-oxo-1,2,3,6-tetrahydropyridin-4-yl)-4-fluorobenzoic acid which was taken into the next step without purification.

Step ii: To a mixture of 6 g of above prepared crude mixture of tert-butyl 8-fluoro-1,6-dioxo-1,2,4,6-tetrahydro-3H-isochromeno[3,4-c]pyridine-3-carboxylate and 2-(1-(tert-butoxycarbonyl)-5-hydroxy-3-oxo-1,2,3,6-tetrahydropyridin-4-yl)-4-fluorobenzoic acid in 30 mL of 1,2-dichlorethane in a sealed tube was added 3.4 g (4.54 mmol, 2.5 eq.) of ammonium acetate and the mixture was heated at 120° C. for 16 h. (Note: Reaction was performed on 2×6 g scale in parallel). On cooling to room temperature, the duplicate reaction mixtures were combined, poured in ice-cold water (200 mL), and extracted with ethyl acetate (2×25 mL). The combined organic extracts were washed with brine (50 mL), dried over anhydrous sodium sulfate, filtered and concentrated under reduced pressure. The resulting crude product was triturated with acetone (50 mL) to afford 3.8 g (1.14 mmol, 28% over two steps) of tert-butyl 8-fluoro-1,6-dioxo-1,4,5,6-tetrahydrobenzo[c][1,7]naphthyridine-3(2H)-carboxylate (IVac). LCMS: m/z found 349.5 [M−H]; ¹H NMR (400 MHz, DMSO-d₆): δ 12.45 (br s, 1H), 9.03-8.97 (m, 1H), 8.12 (dd, 1H), 7.9 (dd, 1H), 4.71 (br s, 2H), 4.18 (br s, 2H), 1.42 (s, 9H).

### tert-Butyl 8-fluoro-1-(methylamino)-6-oxo-1,4,5,6-tetrahydrobenzo[c][1,7]naphthyridine-3(2H)-carboxylate (Vas)

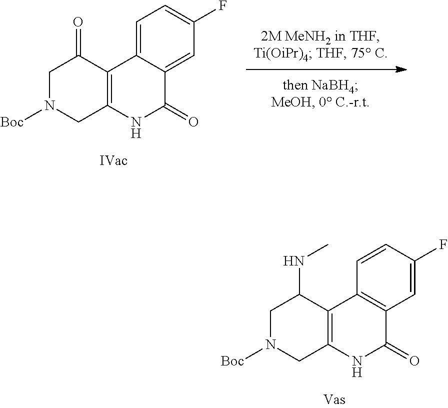 [SMILES: CC(C)(C)OC(=O)N1CC(=O)c2c([nH]c(=O)c3cc(F)ccc23)C1 ; CNC1CN(C(=O)OC(C)(C)C)Cc2[nH]c(=O)c3cc(F)ccc3c21]

To a stirred solution of 2.0 g (6.02 mmol, 1.0 eq.) of tert-butyl 8-fluoro-1,6-dioxo-1,4,5,6-tetrahydrobenzo[c][1,7]naphthyridine-3(2H)-carboxylate (IVac) in 10 mL of THE in sealed tube at room temperature under a nitrogen atmosphere was added 3.6 mL (7.2 mmol, 1.2 eq.) of a 2 M methylamine solution in THF followed by 10 mL (5 vol) of titanium isopropoxide and the reaction mixture was heated at 70° C. for 3 h. The mixture was allowed to cool to room temperature, further cooled to 0° C., and then diluted with methanol (2 mL). To this mixture at 0° C., 0.69 mg (18.64 mmol, 3.0 eq) of NaBH₄ was added portion-wise and then the reaction was continued at room temperature for 2 h. The mixture was then diluted with saturated brine (15 mL) and 10% MeOH in dichloromethane (200 mL). After stirring for 30 min, the heterogeneous mixture was filtered and washed with 10% MeOH in dichloromethane (50 mL). The filtrate was dried over anhydrous sodium sulfate and concentrated under reduced pressure. The resulting crude product was triturated with n-pentane (50 mL), the precipitated solid was collected by filtration and dried under vacuum to afford 1.3 g of tert-butyl 8-fluoro-1-(methylamino)-6-oxo-1,4,5,6-tetrahydrobenzo[c][1,7]naphthyridine-3(2H)-carboxylate (Vas). LCMS: m/z found 348.3 [M+H]⁺.

### 3-(3-Chloro-4-fluorophenyl)-1-(8-fluoro-6-oxo-1,2,3,4,5,6-hexahydrobenzo[c][1,7]naphthyridin-1-yl)-1-methylurea (Compounds 153 and 154)

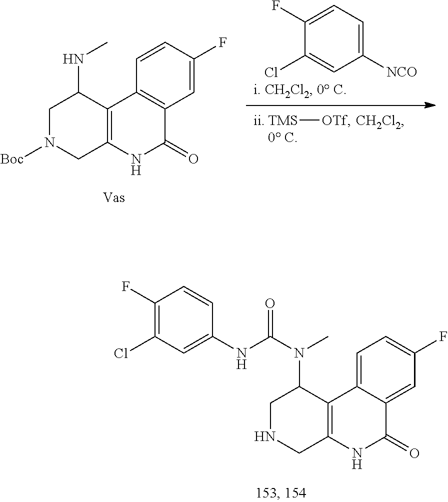

Step i. To a stirred solution of 280 mg (0.81 mmol, 1.0 eq) of tert-butyl 8-fluoro-1-(methylamino)-6-oxo-1,4,5,6-tetrahydrobenzo[c][1,7]naphthyridine-3(2H)-carboxylate (Vas) in 5 mL of dichloromethane at 0° C. was added 0.8 mL (0.645 mmol, 1.0 eq) of 2-chloro-1-fluoro-4-isocyanatobenzene and the resulting mixture was stirred at room temperature for 1 h. The mixture was then diluted with water (20 mL) and extracted with ethyl acetate (2×50 mL). The combined organic extracts were washed with brine (30 mL), dried over anhydrous Na₂SO₄ and concentrated under reduced pressure. The resulting crude product was triturated with diethyl ether (10 mL) to afford 200 mg (0.386 mmol, 47%) of tert-butyl 1-(3-(3-chloro-4-fluorophenyl)-1-methylureido)-8-fluoro-6-oxo-1,4,5,6-tetrahydrobenzo[c][1,7]naphthyridine-3(2H)-carboxylate as a colourless liquid. LCMS: m/z found 517.3 [M−H]; ¹H NMR (400 MHz, DMSO-d₆) at 90° C.: δ 11.57 (br s, 1H), 8.57 (s, 1H), 8.11-8.06 (m, 1H) 7.83 (dd, 1H), 7.52-7.48 (m, 1H), 7.37-7.26 (m, 2H), 5.46 (s, 1H), 3.75 (d, 1H), 3.60 (d, 1H), 3.09-3.02 (m, 2H), 2.80 (s, 3H), 2.75-2.66 (m, 1H), 1.4 (s, 9H).

Step ii. To a stirred solution of 200 mg (0.386 mmol, 1.0 eq) of tert-butyl 1-(3-(3-chloro-4-fluorophenyl)-1-methylureido)-8-fluoro-6-oxo-1,4,5,6-tetrahydrobenzo[c][1,7]naphthyridine-3(2H)-carboxylate, obtained in Step i, in 5 mL of dichloromethane at 0° C. was added 171 mg (0.77 mmol, 2.0 eq) of trimethylsilyl trifluoromethanesulfonate and the resulting mixture was stirred at room temperature for 1 h. The volatiles were then removed under reduced pressure. The resulting residue was diluted with saturated NaHCO₃ solution (20 mL) and precipitated solid was collected by filtration and dried under vacuum; the crude was triturated with dichloromethane (2 ml) and n-pentane (10 mL) to afford 130 mg (0.317 mmol, 75%) of 3-(3-chloro-4-fluorophenyl)-1-(8-fluoro-6-oxo-1,2,3,4,5,6-hexahydrobenzo[c][1,7]naphthyridin-1-yl)-1-methylurea as a white solid. The enantiomers were subsequently separated by preparative SFC: Method isocratic, Mobile phase MeOH: CO₂—30:70. Column: Chiralcel OD-H (30×250 mm), 5μ, flow rate: 100 g/min.

Enantiomer I (Compound 153): LCMS: m/z found 419.2/421.2 [M+H]⁺, RT=3.04 min, (Method A); ¹H NMR (400 MHz, DMSO-d₆) at 90° C.: δ 11.58 (br s, 1H), 8.58 (br s, 1H), 8.11-8.06 (m, 1H) 7.83 (dd, 1H), 7.51-7.47 (m, 1H), 7.37-7.30 (m, 2H), 5.33 (s, 1H), 3.75 (d, 1H), 3.60 (d, 1H), 3.09-3.02 (m, 2H), 2.80 (s, 3H), 2.75-2.66 (m, 1H); Chiral analytical SFC: RT=2.42 min, Column: Chiralcel OD-H (4.6×150 mm) 3 μm, 40% methanol, Flow rate: 3 g/min.

Enantiomer II (Compound 154): LCMS: m/z found 419.2/421.2 [M+H]⁺, RT=3.04 min, (Method A); ¹H NMR (400 MHz, DMSO-d₆) at 90° C.: δ 11.57 (br s, 1H), 8.57 (br s, 1H), 8.11-8.06 (m, 1H) 7.83 (dd, 1H), 7.52-7.48 (m, 1H), 7.37-7.26 (m, 2H), 5.46 (s, 1H), 3.75 (d, 1H), 3.60 (d, 1H), 3.09-3.02 (m, 2H), 2.80 (s, 3H), 2.75-2.66 (m, 1H); Chiral analytical SFC: RT=3.2 min, Column: Chiralcel OD-H (4.6×150 mm) 3 μm, 40% methanol, Flow rate: 3 g/min.

### 3-(3-Cyano-4-fluorophenyl)-1-(8-fluoro-6-oxo-1,2,3,4,5,6-hexahydrobenzo[c][1,7]naphthyridin-1-yl)-1-methylurea (Compounds 161 and 162)

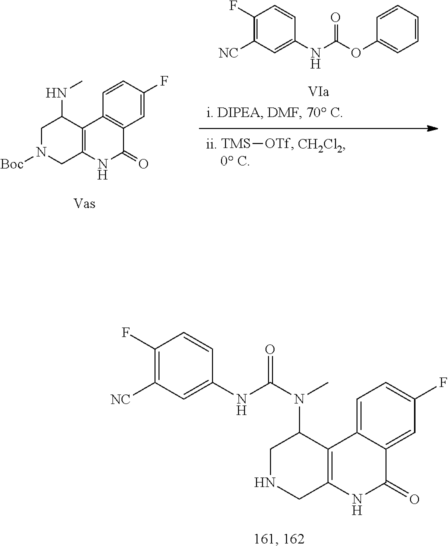 [SMILES: N#Cc1cc(NC(=O)Oc2ccccc2)ccc1F ; CNC1CN(C(=O)OC(C)(C)C)Cc2[nH]c(=O)c3cc(F)ccc3c21 ; C[Si](C)(C)OS(=O)(=O)C(F)(F)F ; CN(C(=O)Nc1ccc(F)c(C#N)c1)C1CNCc2[nH]c(=O)c3cc(F)ccc3c21]

Step i. To a stirred solution of of tert-butyl 8-fluoro-1-(methylamino)-6-oxo-1,4,5,6-tetrahydrobenzo[c][1,7]naphthyridine-3(2H)-carboxylate (Vas, 250 mg, 0.97 mmol, 1.0 eq) in 5 mL of DMF at room temperature, 0.52 mL (4.1 mmol, 2.91 eq) of DIPEA and 338 mg (0.97 mmol, 1.0 eq) of phenyl (3-cyano-4-fluorophenyl)carbamate (VIa) were added, and the resulting reaction mixture was stirred at 70° C. for 3 h. The mixture was then diluted with cold water (15 mL) and stirred for 30 minutes. The precipitated solid was filtered to afford 200 mg (0.37 mmol, 54% yield) of tert-butyl 1-(3-(3-cyano-4-fluorophenyl)-1-methylureido)-8-fluoro-6-oxo-1,4,5,6-tetrahydrobenzo[c][1,7]naphthyridine-3(2H)-carboxylate as an off white solid. LCMS: m/z found 508.3 [M−H]—.

Step ii. To a stirred solution of of tert-butyl 1-(3-(3-chloro-4-fluorophenyl)-1-methylureido)-8-fluoro-6-oxo-1,4,5,6-tetrahydrobenzo[c][1,7]naphthyridine-3(2H)-carboxylate (200 mg, 0.392 mmol, 1.0 eq) in 4 mL of dichloromethane at 0° C., trimethylsilyl trifluoromethanesulfonate (0.145 mL, 0.78 mmol, 2 eq) was added and the resulting reaction mixture was stirred at room temperature for 1 h. The volatiles were then removed under reduced pressure. The resulting residue was diluted with saturated NaHCO₃ solution (15 mL), precipitated solid was collected by filtration and dried under vacuum to afford 120 mg (0.293 mmol, 72% yield) of 3-(3-cyano-4-fluorophenyl)-1-(8-fluoro-6-oxo-1,2,3,4,5,6-hexahydrobenzo[c][1,7]naphthyridin-1-yl)-1-methylurea. The enantiomers were subsequently separated by preparative SFC: Method isocratic, Mobile phase (0.2% 7 M Methanolic Ammonia in Acetonitrile:MeOH (1:1) v/v): CO₂—45:55. Column: Chiralpak-IE (30×250 mm), 5μ, flow rate: 100 g/min.

Enantiomer I (Compound 161): LCMS: m/z found 410.2 [M+H]⁺, RT=2.61 min, (Method A); ¹H NMR (400 MHz, DMSO-d₆): δ 11.65 (br s, 1H), 8.96 (br s, 1H), 8.11-8.04 (m, 1H) 7.91-7.84 (m, 1H), 7.86 (t, 1H), 7.65-7.60 (t, 2H), 7.4 (d, 1H), 5.42 (s, 1H), 4.9 (d, 1H), 4.3 (d, 1H), 3.70 (s, 1H), 3.07 (d, 2H), 2.61 (s, 3H), 2.75-2.66 (m, 1H); Chiral analytical SFC: RT=1.89 min, Column: Chiralpak IE-3 (4.6×150 mm) 3 μm, 40% (0.2% 7M Methanolic Ammonia in ACN:MeOH (1:1)), Flow rate: 3 g/min.

Enantiomer II (Compound 162): LCMS: m/z found 410.2 [M+H]⁺, RT=2.61 min, min, (Method A); ¹H NMR (400 MHz, DMSO-d₆): δ 11.65 (br s, 1H), 8.96 (br s, 1H), 8.11-8.04 (m, 1H) 7.91-7.84 (m, 1H), 7.86 (t, 1H), 7.65-7.60 (t, 2H), 7.4 (d, 1H), 5.42 (s, 1H), 4.9 (d, 1H), 4.3 (d, 1H), 3.70 (s, 1H), 3.07 (d, 2H), 2.61 (s, 3H), 2.75-2.66 (m, 1H); Chiral analytical SFC: RT=2.92 min, Column: Chiralcel OD-H (4.6×150 mm) 3 μm, 40% (0.2% 7M Methanolic Ammonia in ACN:MeOH (1:1)), Flow rate: 3 g/min.

### 3-(2-((tert-Butyldimethylsilyl)oxy)ethyl)-8-fluoro-3,4-dihydrobenzo[c][1,7]naphthyridine-1,6(2H,5H)-dione (IVad)

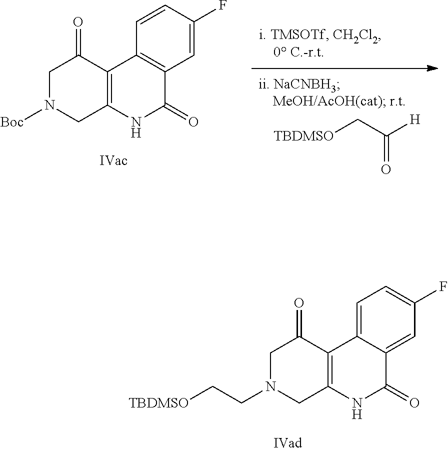 [SMILES: CC(C)(C)OC(=O)N1CC(=O)c2c([nH]c(=O)c3cc(F)ccc23)C1 ; CC(C)(C)[Si](C)(C)OCC=O ; CC(C)(C)[Si](C)(C)OCCN1CC(=O)c2c([nH]c(=O)c3cc(F)ccc23)C1]

Step i: To a stirred solution of 2.0 g (6.024 mmol, 1.0 eq) of tert-butyl 8-fluoro-1,6-dioxo-1,4,5,6-tetrahydrobenzo[c][1,7]naphthyridine-3(2H)-carboxylate (IVac) in 20 mL of dichloromethane at 0° C. was added 1.6 mL (9.03 mmol, 1.5 eq) of trimethylsilyl trifluoromethanesulfonate and the resulting reaction mixture was stirred at room temperature for 1 h. The volatiles were removed under reduced pressure and the residue was triturated with saturated sodium bicarbonate solution (20 mL). The solids were collected by filtration and dried under vacuum to afford 1.3 g (5.85 mmol, 93%) of 8-fluoro-3,4-dihydrobenzo[c][1,7]naphthyridine-1,6-(2H,5H)-dione as a brown solid. LCMS: m/z found 233.4 [M−H]⁻.

Step ii: To a stirred solution of 1.75 g (7.54 mmol, 1.0 eq.) of 8-fluoro-3,4-dihydrobenzo[c][1,7]naphthyridine-1,6(2H,5H)-dione in 17.5 mL of methanol, 1.96 g (11.31, 1.5 eq.) of 2-((tert-butyldimethylsilyl)oxy)acetaldehyde, 0.87 mL of acetic acid and 0.95 g (15.08 mmol, 2.0 eq.) of sodium cyanoborohydride were added and the resulting reaction mixture was stirred at room temperature for 16 h. The reaction mixture was then concentrated, and the residue was diluted with water (50 mL) and stirred for 30 min. The precipitated solid was collected by filtration and dried under vacuum to afford 1.3 g (3.3 mmol, 45%) of 3-(2-((tert-butyldimethylsilyl)oxy)ethyl)-8-fluoro-3,4-dihydrobenzo[c][1,7]naphthyridine-1,6(2H,5H)-dione (IVad). LCMS: m/z found 391.17 [M+H]⁻.

### 3-(2-((tert-Butyldimethylsilyl)oxy)ethyl)-8-fluoro-1-(methylamino)-1,3,4,5-tetrahydrobenzo[c][1,7]naphthyridin-6(2H)-one (Vat)

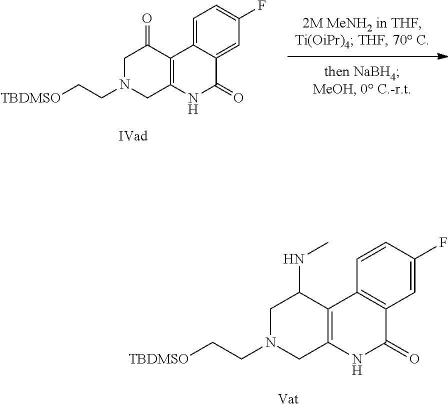 [SMILES: CC(C)(C)[Si](C)(C)OCCN1CC(=O)c2c([nH]c(=O)c3cc(F)ccc23)C1 ; CNC1CN(CCO[Si](C)(C)C(C)(C)C)Cc2[nH]c(=O)c3cc(F)ccc3c21]

3-(2-((tert-Butyldimethylsilyl)oxy)ethyl)-8-fluoro-1-(methylamino)-1,3,4,5-tetrahydrobenzo[c][1,7]naphthyridin-6(2H)-one was prepared from methylamine and 3-(2-((tert-butyldimethylsilyl)oxy)ethyl)-8-fluoro-3,4-dihydrobenzo[c][1,7]naphthyridine-1,6(2H,5H)-dione (IVad) by a procedure similar to the one described above for Vas. LCMS: m/z found 406.5 [M−H]⁻.

### 3-(3-Cyano-4-fluorophenyl)-1-(8-fluoro-3-(2-hydroxyethyl)-6-oxo-1,2,3,4,5,6-hexahydrobenzo[c][1,7]naphthyridin-1-yl)-1-methylurea(Compounds 171 and 172)

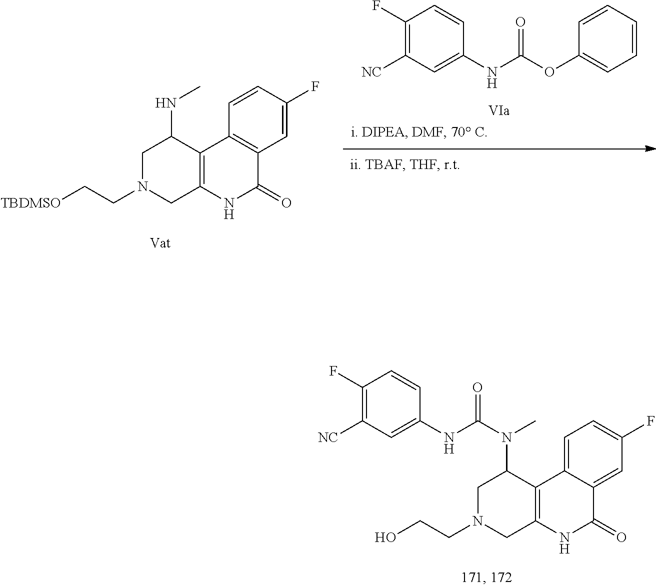 [SMILES: N#Cc1cc(NC(=O)Oc2ccccc2)ccc1F ; CNC1CN(CCO[Si](C)(C)C(C)(C)C)Cc2[nH]c(=O)c3cc(F)ccc3c21 ; CN(C(=O)Nc1ccc(F)c(C#N)c1)C1CN(CCO)Cc2[nH]c(=O)c3cc(F)ccc3c21]

Step i. To a stirred solution of 3-(2-((tert-butyldimethylsilyl)oxy)ethyl)-8-fluoro-1-(methylamino)-1,3,4,5-tetrahydrobenzo[c][1,7]naphthyridin-6(2H)-one (Vat, 350 mg, 0.86 mmol, 1.0 eq) in 7 mL of DMF at room temperature, 0.46 mL (2.58 mmol, 3.0 eq) of DIPEA, 155 mg (0.60 mmol, 0.7 eq) phenyl (3-cyano-4-fluorophenyl)carbamate (VIa) were added and the resulting reaction mixture was stirred at 70° C. for 3 h. The reaction mixture was then diluted with cold water (25 mL) and stirred for 30 minutes. The precipitated solid was filtered and dried to afford 450 mg (0.81 mmol, 55% yield) of 1-(3-(2-((tert-butyldimethylsilyl)oxy)ethyl)-8-fluoro-6-oxo-1,2,3,4,5,6-hexahydrobenzo[c][1,7]naphthyridin-1-yl)-3-(3-cyano-4-fluorophenyl)-1-methylurea as a pale brown solid. LCMS: m/z found 568.50 [M+H]⁻.

Step ii. To a stirred solution of 1-(3-(2-((tert-butyldimethylsilyl)oxy)ethyl)-8-fluoro-6-oxo-1,2,3,4,5,6-hexahydrobenzo[c][1,7]naphthyridin-1-yl)-3-(3-cyano-4-fluorophenyl)-1-methylurea (450 mg, 0.811 mmol, 1.0 eq) in 9 mL of THF at 0° C., TBAF (1.62 mL, 1.62 mmol, 2.0 eq) was added and the reaction was continued at room temperature for 2 h. After completion of the reaction (monitored by TLC), the reaction was quenched with MeOH (1 mL) and then organic volatiles were evaporated under reduced pressure. The residue was diluted with water (20 mL) and stirred for 30 minutes. The precipitated solid was collected by filtration and dried to afford 3-(3-cyano-4-fluorophenyl)-1-(8-fluoro-3-(2-hydroxyethyl)-6-oxo-1,2,3,4,5,6-hexahydrobenzo[c][1,7]naphthyridin-1-yl)-1-methylurea (180 mg, 85%). The enantiomers were subsequently separated by preparative SFC: Method isocratic, Mobile phase MeOH:CO₂—40:60. Column: Chiralcel-OX-H (30×250 mm), 5μ, flow rate: 100 g/min.

Enantiomer I (Compound 171): LCMS: m/z found 454.3 [M+H]⁺, RT=2.87 min, (Method A); ¹H NMR (400 MHz, DMSO-d₆): δ 11.52 (br s, 1H), 8.6 (br s, 1H), 8.09 (s, 1H), 7.86-7.80 (d, 2H) 7.64 (t, 1H), 7.58-7.48 (d, 2H), 5.5 (s, 1H), 4.53 (t, 1H), 3.88 (d, 1H), 3.53-3.58 (m, 2H), 3.17 (d, 1H), 3.02 (d, 1H), 2.83 (s, 3H), 2.73-2.67 (m, 1H), 2.59-2.51 (m, 2H); Chiral analytical SFC: RT=2.51 min, Column: Chiralcel OX-3 (4.6×150 mm) 3 μm, 30% (0.5% DEA in Methanol), Flow rate: 3 g/min.

Enantiomer II (Compound 172): LCMS: m/z found 454.3 [M+H]⁺, RT=2.87 min, min, (Method A); ¹H NMR (400 MHz, DMSO-d₆): δ 11.52 (br s, 1H), 8.6 (br s, 1H), 8.09 (s, 1H), 7.86-7.80 (d, 2H) 7.64 (t, 1H), 7.58-7.48 (d, 2H), 5.5 (s, 1H), 4.53 (t, 1H), 3.88 (d, 1H), 3.53-3.58 (m, 2H), 3.17 (d, 1H), 3.02 (d, 1H), 2.83 (s, 3H), 2.73-2.67 (m, 1H), 2.59-2.51 (m, 2H); Chiral analytical SFC: RT=3.49 min, Column: Chiralcel OX-3 (4.6×150 mm) 3 μm, 30% (0.5% DEA in Methanol), Flow rate: 3 g/min.

### 8-Fluoro-3-methyl-3,4-dihydrobenzo[c][1,7]naphthyridine-1,6(2H,5H)-dione (IVae)

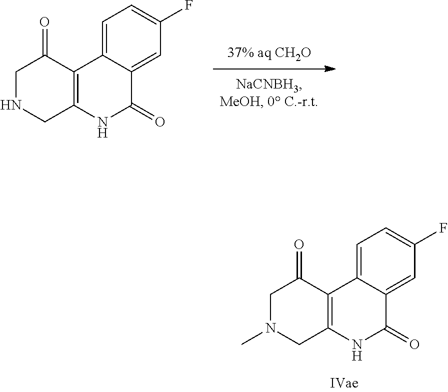 [SMILES: O=C1CNCc2[nH]c(=O)c3cc(F)ccc3c21 ; CN1CC(=O)c2c([nH]c(=O)c3cc(F)ccc23)C1]

To a stirred solution of 1.0 g (4.31 mmol, 1.0 eq.) of 8,9-difluoro-3,4-dihydrobenzo[c][1,7]naphthyridine-1,6(2H,5H)-dione (obtained as described above for IVad, Step i) in 10 mL of methanol were added 5 mL of 37% aqueous solution of formaldehyde and 0.54 g (8.62 mmol, 2.0 eq) of sodium cyanoborohydride and the resulting mixture was stirred at room temperature for 16 h. The mixture was then diluted with water (150 mL) and extracted with ethyl acetate (3×150 mL). The combined organic extracts were dried over anhydrous sodium sulfate and concentrated under reduced pressure to afford crude 0.75 g (3.17 mmol, 78%) of 8-fluoro-3-methyl-3,4-dihydrobenzo[c][1,7]naphthyridine-1,6(2H,5H)-dione (IVae). LCMS: m/z found 247.19 [M+H]⁺.

### 8-Fluoro-3-methyl-1-(methylamino)-1,3,4,5-tetrahydrobenzo[c][1,7]naphthyridin-6(2H)-one (Vau)

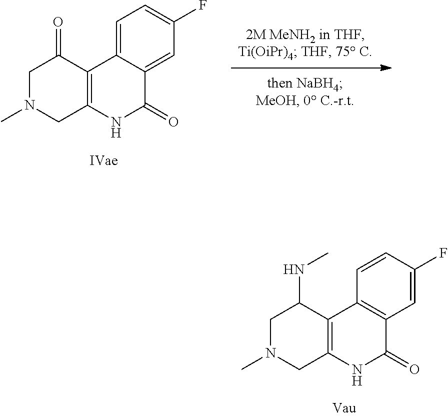 [SMILES: CN1CC(=O)c2c([nH]c(=O)c3cc(F)ccc23)C1 ; CNC1CN(C)Cc2[nH]c(=O)c3cc(F)ccc3c21]

Racemic 8-fluoro-3-methyl-1-(methylamino)-1,3,4,5-tetrahydrobenzo[c][1,7]naphthyridin-6(2H)-one was synthesized in an analogous manner as described above for Vas, from 8-fluoro-3-methyl-3,4-dihydrobenzo[c][1,7]naphthyridine-1,6(2H,5H)-dione (IVae) and methylamine. LCMS: m/z found 262.29 [M+H]⁺.

### 3-(3-Chloro-4-fluorophenyl)-1-(8-fluoro-3-methyl-6-oxo-1,2,3,4,5,6-hexahydrobenzo[c][1,7]naphthyridin-1-yl)-1-methylurea (Compounds 155 and 156)

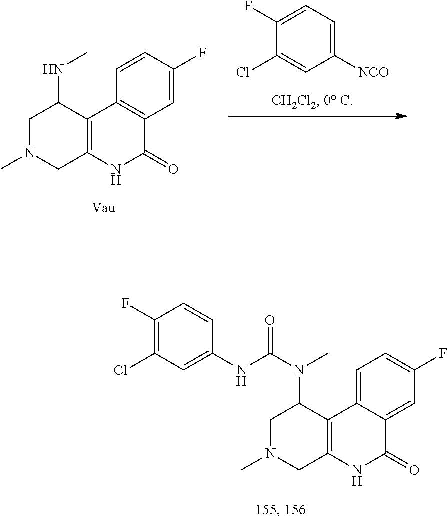

Racemic 3-(3-chloro-4-fluorophenyl)-1-(8-fluoro-3-methyl-6-oxo-1,2,3,4,5,6-hexahydrobenzo[c][1,7]naphthyridin-1-yl)-1-methylurea was synthesized from 8-fluoro-3-methyl-1-(methylamino)-1,3,4,5-tetrahydrobenzo[c][1,7]naphthyridin-6(2H)-one (Vau) in an analogous manner as described above for Compounds 153 and 154 (Step i). The enantiomers were subsequently separated by preparative SFC: Method isocratic, Mobile phase MeOH:CO₂—40:60. Column: Chiralcel OD-H (30×250 mm), 5μ, flow rate: 60 g/min.

Enantiomer I (Compound 155): LCMS: m/z found 433.2/435.2 [M+H]⁺, RT=3.02 min, (Method A); ¹H NMR (400 MHz, DMSO-d₆): δ 11.57 (br s, 1H), 8.51 (br s, 1H), 7.88-7.85 (m, 2H), 7.70-7.65 (m, 1H), 7.51-7.47 (m, 2H), 7.31 (t, 1H), 5.52 (br s, 1H), 3.66 (d, 1H), 3.01 (d, 1H), 2.91 (d, 1H), 2.77 (s, 3H), 2.61-2.57 (m, 1H), 2.33 (s, 3H); Chiral analytical SFC: RT=1.10 min, Column: Chiralcel OD-3 (4.6×150 mm) 3 μm, 40% methanol, Flow rate: 3 g/min.

Enantiomer II (Compound 156): LCMS: m/z found 433.2/435.2 [M+H]⁺, RT=3.02 min, (Method A); ¹H NMR (400 MHz, DMSO-d₆): δ 11.57 (br s, 1H), 8.51 (br s, 1H), 7.89-7.85 (m, 2H), 7.69-7.64 (m, 1H), 7.50-7.47 (m, 2H), 7.31 (t, 1H), 5.52 (br s, 1H), 3.66 (d, 1H), 3.01 (d, 1H), 2.91 (d, 1H), 2.77 (s, 3H), 2.61-2.57 (m, 1H), 2.33 (s, 3H); Chiral analytical SFC: RT=1.55 min, Column: Chiralcel OD-3 (4.6×150 mm) 3 μm, 40% methanol, Flow rate: 3 g/min.

### 3-(3-Cyano-4-fluorophenyl)-1-(8-fluoro-3-methyl-6-oxo-1,2,3,4,5,6-hexahydrobenzo[c][1,7]naphthyridin-1-yl)-1-methylurea (Compounds 163 and 164)

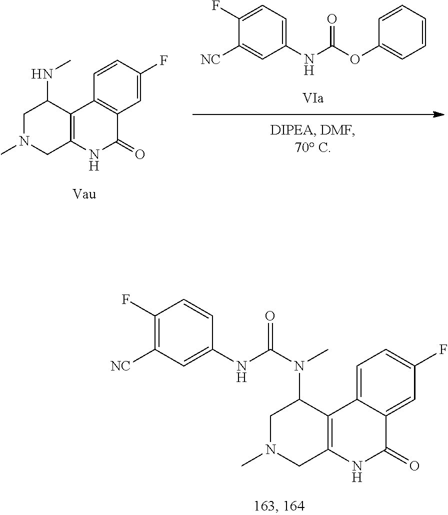 [SMILES: N#Cc1cc(NC(=O)Oc2ccccc2)ccc1F ; CNC1CN(C)Cc2[nH]c(=O)c3cc(F)ccc3c21 ; CN1Cc2[nH]c(=O)c3cc(F)ccc3c2C(N(C)C(=O)Nc2ccc(F)c(C#N)c2)C1]

Racemic 3-(3-cyano-4-fluorophenyl)-1-(8-fluoro-3-methyl-6-oxo-1,2,3,4,5,6-hexahydrobenzo[c][1,7]naphthyridin-1-yl)-1-methylurea was synthesized from 8-fluoro-3-methyl-1-(methylamino)-1,3,4,5-tetrahydrobenzo[c][1,7]naphthyridin-6(2H)-one (Vau) in an analogous manner as described above for Compounds 161 and 162 (Step i). The enantiomers were subsequently separated by preparative SFC: Method isocratic, Mobile phase MeOH:CO₂—35:65. Column: Chiralpak IC (30×250 mm), 5μ, flow rate: 60 g/min.

Enantiomer I (Compound 163): LCMS: m/z found 424.2 [M+H]⁺, RT=2.58 min, (Method A); ¹H NMR (400 MHz, DMSO-d₆): δ 11.58 (br s, 1H), 8.69 (brs, 1H), 8.09 (brs, 1H), 7.88-7.82 (m, 2H), 7.70-7.64 (m, 1H), 7.51-7.43 (m, 2H), 5.52 (brs, 1H), 3.66 (d, 1H), 3.01 (d, 1H), 2.92 (d, 1H), 2.78 (s, 3H), 2.61-2.58 (m, 1H), 2.32 (s, 3H); Chiral analytical SFC: RT=2.45 min, Column: Chiralpak IC-3 (4.6×150 mm) 3 μm, 30% (0.5 DEA in methanol), Flow rate: 3 g/min.

Enantiomer II (Compound 164): LCMS: m/z found 424.2 [M+H]⁺, RT=2.58 min, min, (Method A); ¹H NMR (400 MHz, DMSO-d₆): δ 11.58 (br s, 1H), 8.69 (brs, 1H), 8.09 (brs, 1H), 7.88-7.82 (m, 2H), 7.70-7.64 (m, 1H), 7.51-7.43 (m, 2H), 5.52 (brs, 1H), 3.66 (d, 1H), 3.01 (d, 1H), 2.92 (d, 1H), 2.78 (s, 3H), 2.61-2.58 (m, 1H), 2.32 (s, 3H); Chiral analytical SFC: RT=3.23 min, Column: Chiralpak IC-3 (4.6×150 mm) 3 μm, 30% (0.5 DEA in methanol), Flow rate: 3 g/min.

### 3-Acetyl-8-fluoro-3,4-dihydrobenzo[c][1,7]naphthyridine-1,6(2H,5H)-dione (IVaf)

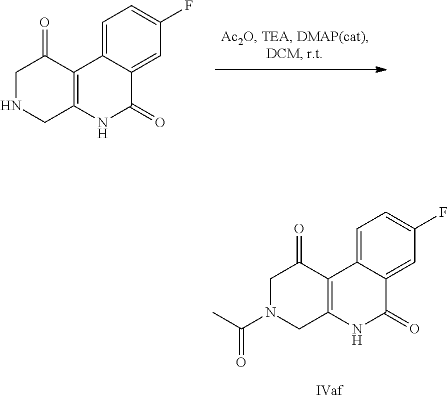 [SMILES: O=C1CNCc2[nH]c(=O)c3cc(F)ccc3c21 ; CC(=O)N1CC(=O)c2c([nH]c(=O)c3cc(F)ccc23)C1]

To a stirred solution of 0.5 g (2.16 mmol, 3.0 eq.) of fluoro-3,4-dihydrobenzo[c][1,7]naphthyridine-1,6(2H,5H)-dione (obtained as described above for IVad, Step i) in 5 mL of dichloromethane, were added 0.6 mL (4.31 mmol, 2.0 eq.) of triethylamine and 0.20 mL (2.16 mmol, 1.0 eq.) of acetic anhydride and the mixture was stirred at room temperature for 4 h. The reaction mixture was concentrated and washed with water (20 mL) to afford 0.4 g of 3-acetyl-8-fluoro-3,4-dihydrobenzo[c][1,7]naphthyridine-1,6(2H,5H)-dione (IVaf) as a pale yellow solid. LCMS: m/z found 275.3 [M+H]⁺. ¹H NMR (400 MHz, DMSO-d₆): δ 9.13-9.09 (m, 1H), 7.89-7.86 (m, 1H), 7.73-7.68 (1H), 4.80-479 (d, 2H), 4.34-4.28 (d 2H), 2.13-2.10 (d, 3H).

### 3-Acetyl-8-fluoro-1-(methylamino)-1,3,4,5-tetrahydrobenzo[c][1,7]naphthyridin-6(2H)-one (Vav)

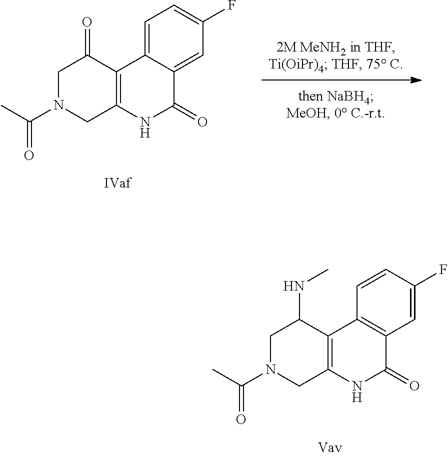 [SMILES: CC(=O)N1CC(=O)c2c([nH]c(=O)c3cc(F)ccc23)C1 ; CNC1CN(C(C)=O)Cc2[nH]c(=O)c3cc(F)ccc3c21]

Racemic 3-acetyl-8-fluoro-1-(methylamino)-1,3,4,5-tetrahydrobenzo[c][1,7]naphthyridin-6(2H)-one was synthesized in an analogous manner as described above for Vas, from 3-acetyl-8-fluoro-3,4-dihydrobenzo[c][1,7]naphthyridine-1,6(2H,5H)-dione (IVaf) and methylamine. LCMS: m/z found 288.4 [M−H]⁺.

### 1-(3-Acetyl-8-fluoro-6-oxo-1,2,3,4,5,6-hexahydrobenzo[c][1,7]naphthyridin-1-yl)-3-(3-chloro-4-fluorophenyl)-1-methylurea (Compounds 157 and 158)

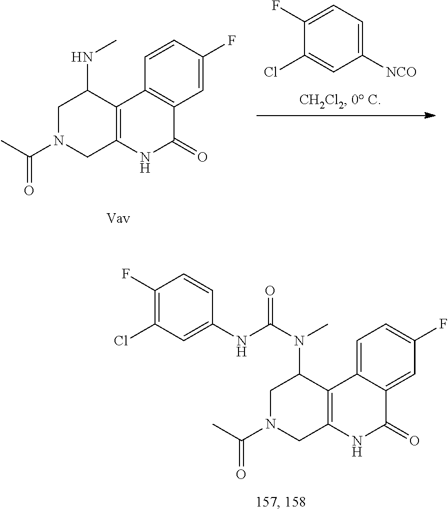

Racemic 1-(3-acetyl-8-fluoro-6-oxo-1,2,3,4,5,6-hexahydrobenzo[c][1,7]naphthyridin-1-yl)-3-(3-chloro-4-fluorophenyl)-1-methylurea was synthesized from 3-acetyl-8-fluoro-1-(methylamino)-1,3,4,5-tetrahydrobenzo[c][1,7]naphthyridin-6(2H)-one (Vav) in an analogous manner as described above. The enantiomers were subsequently separated by preparative SFC: Method isocratic, Mobile phase MeOH:CO₂—40:60. Column: Chiralpak OJ (30×250 mm), 5μ, flow rate: 100 g/min.

Enantiomer I (Compound 157): LCMS: m/z found 461.2 [M+H]⁺, RT=3.85 min, (Method A); ¹H NMR (400 MHz, DMSO-d₆): δ 11.7 (br s, 1H), 8.609-8.50 (d, 1H), 7.90-7.87 (m, 2H), 7.71-7.68 (m, 1H), 7.57-7.48 (m 2H), 7.35-7.31 (t, 1H), 5.61-5.5 (d, 1H), 5.11-4.71 (m, 1H), 4.59-4.3 (m, 1H) 4.10-3.9 (m, 1H), 3.61-3.5 (m, 1H), 2.61 (s 3H), 2.11 (s, 3H); Chiral analytical SFC: RT=2.56 min, Column: Chiralpak OJ-3 (4.6×150 mm) 3 μm, 40% methanol, Flow rate: 3 g/min.

Enantiomer II (Compound 158): LCMS: m/z m/z found 461.2 [M+H]⁺, RT=3.85 min, (Method A); ¹H NMR (400 MHz, DMSO-d₆): δ 11.7 (br s, 1H), 8.60-8.50 (d, 1H), 7.91-7.87 (m, 2H), 7.72-7.67 (m, 1H), 7.57-7.49 (m 2H), 7.35-7.31 (t, 1H), 5.61-5.5 (d, 1H), 5.11-4.71 (m, 1H), 4.59-4.3 (m, 1H) 4.10-3.9 (m, 1H), 3.61-3.5 (m, 1H), 2.61 (s 3H), 2.11 (s, 3H); Chiral analytical SFC: RT=3.60 min, Column: Chiralpak OJ-3 (4.6×150 mm) 3 μm, 40% methanol, Flow rate: 3 g/min.

### 1-(3-Acetyl-8-fluoro-6-oxo-1,2,3,4,5,6-hexahydrobenzo[c][1,7]naphthyridin-1-yl)-3-(3-cyano-4-fluorophenyl)-1-methylurea (Compounds 165 and 166)

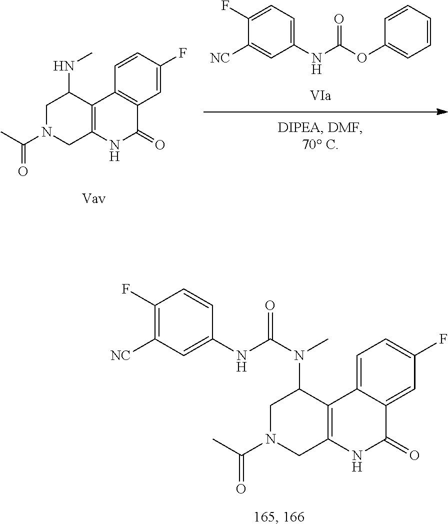 [SMILES: CNC1CN(C(C)=O)Cc2[nH]c(=O)c3cc(F)ccc3c21 ; N#Cc1cc(NC(=O)Oc2ccccc2)ccc1F ; CC(=O)N1Cc2[nH]c(=O)c3cc(F)ccc3c2C(N(C)C(=O)Nc2ccc(F)c(C#N)c2)C1]

Racemic 1-(3-acetyl-8-fluoro-6-oxo-1,2,3,4,5,6-hexahydrobenzo[c][1,7]naphthyridin-1-yl)-3-(3-cyano-4-fluorophenyl)-1-methylurea from 3-acetyl-8-fluoro-1-(methylamino)-1,3,4,5-tetrahydrobenzo[c][1,7]naphthyridin-6(2H)-one (Vav) in an analogous manner as described above. The enantiomers were subsequently separated by preparative SFC: Method isocratic, Mobile phase MeOH:CO₂—40:60. Column: Chiralpak-IC (30×250 mm), 5μ, flow rate: 100 g/min.

Enantiomer I (Compound 165): LCMS: m/z found 452.2 [M+H]⁺, RT=3.36 min, (Method A); ¹H NMR (400 MHz, DMSO-d₆): δ 11.78 (br s, 1H), 8.01-7.98 (m, 1H), 7.92-7.85 (m, 2H), 7.636-7.559 (m, 2H), 7.41-7.37 (t, 1H), 8.79-8.70 (m, 1H), 8.14-8.06 (m, 2H) 7.92-7.89 (m, 1H), 7.50-7.34 (m, 2H), 7.37-7.30 (m, 2H), 5.58 (s, 1H), 5.04-3.62 (m, 4H) 2.61 (s 3H) 2.09 (s 3H); Chiral analytical SFC: RT=3.96 min, Column: Chiralpak IC-3 (4.6×150 mm) 3 μm, 40% methanol, Flow rate: 3 g/min.

Enantiomer II (Compound 166): LCMS: m/z m/z found 452.2 [M+H]⁺, RT=3.36 min, (Method A); ¹H NMR (400 MHz, DMSO-d₆): δ 11.78 (br s, 1H), 8.01-7.98 (m, 1H), 7.92-7.85 (m, 2H), 7.636-7.559 (m, 2H), 7.41-7.37 (t, 1H), 8.79-8.70 (m, 1H), 8.14-8.06 (m, 2H) 7.92-7.89 (m, 1H), 7.50-7.34 (m, 2H), 7.37-7.30 (m, 2H), 5.58 (s, 1H), 5.04-3.62 (m, 4H) 2.61 (s 3H) 2.09 (s 3H); Chiral analytical SFC: RT=5.40 min, Column: Chiralpak IC-3 (4.6×150 mm) 3 μm, 40% methanol, Flow rate: 3 g/min. tert-Butyl 8,9-difluoro-1,6-dioxo-1,4,5,6-tetrahydrobenzo[c][1,7]naphthyridine-3(2H)-carboxylate (IVag)

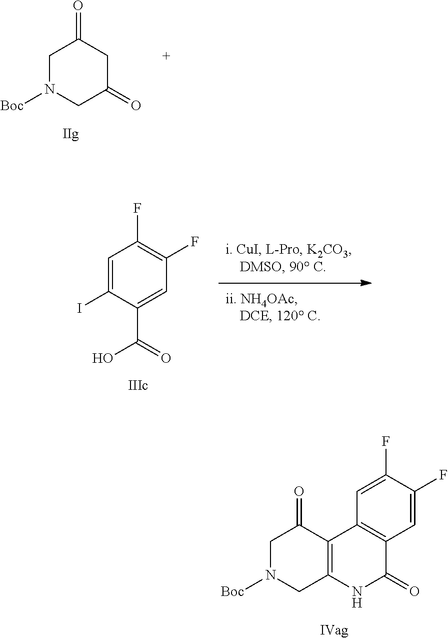 [SMILES: CC(C)(C)OC(=O)N1CC(=O)CC(=O)C1 ; O=C(O)c1cc(F)c(F)cc1I ; CC(C)(C)OC(=O)N1CC(=O)c2c([nH]c(=O)c3cc(F)c(F)cc23)C1]

tert-Butyl 8,9-difluoro-1,6-dioxo-1,4,5,6-tetrahydrobenzo[c][1,7]naphthyridine-3(2H)-carboxylate was synthesized in an analogous manner as described above for IVac, from tert-butyl 3,5-dioxopiperidine-1-carboxylate (Hg) and 4,5-difluoro-2-iodo-benzoic acid (IIIc). ¹H NMR (400 MHz, DMSO-d₆): δ 12.45 (br s, 1H), 9.03-8.97 (m, 1H), 8.12 (dd, 1H), 4.71 (br s, 2H), 4.18 (br s, 2H), 1.42 (s, 9H).

### tert-Butyl 8,9-difluoro-1-(methylamino)-6-oxo-1,4,5,6-tetrahydrobenzo[c][1,7]naphthyridine-3(2H)-carboxylate (Vaw)

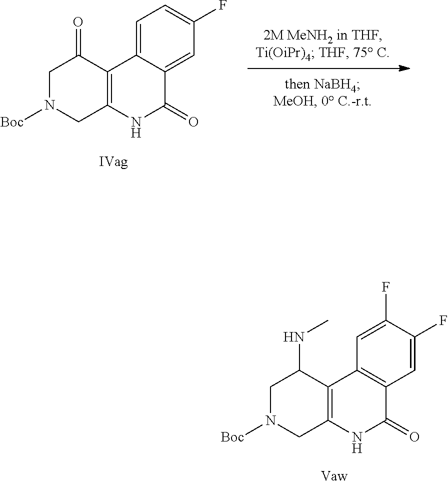 [SMILES: CC(C)(C)OC(=O)N1CC(=O)c2c([nH]c(=O)c3cc(F)ccc23)C1 ; CNC1CN(C(=O)OC(C)(C)C)Cc2[nH]c(=O)c3cc(F)c(F)cc3c21]

Racemic tert-butyl 8,9-difluoro-1-(methylamino)-6-oxo-1,4,5,6-tetrahydrobenzo[c][1,7]naphthyridine-3(2H)-carboxylate was synthesized in an analogous manner as described above for Vas, from tert-butyl 8,9-difluoro-1,6-dioxo-1,4,5,6-tetrahydrobenzo[c][1,7]naphthyridine-3(2H)-carboxylate (IVag) and methylamine. LCMS: m/z found 366.3 [M+H]⁺.

### 3-(3-Chloro-4-fluorophenyl)-1-(8,9-difluoro-6-oxo-1,2,3,4,5,6-hexahydrobenzo[c][1,7]naphthyridin-1-yl)-1-methylurea (Compounds 149 and 150)

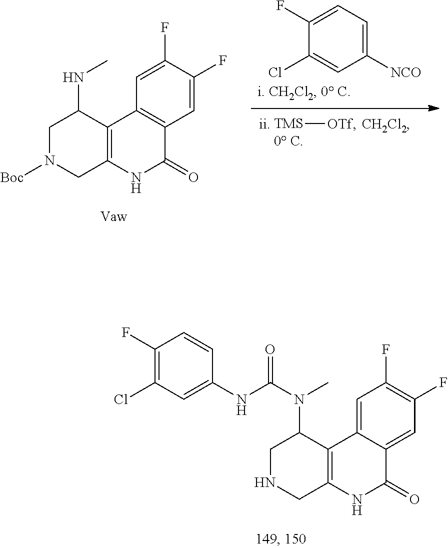

Racemic 3-(3-chloro-4-fluorophenyl)-1-(8,9-difluoro-6-oxo-1,2,3,4,5,6-hexahydrobenzo[c][1,7]naphthyridin-1-yl)-1-methylurea was synthesized from tert-butyl 8,9-difluoro-1-(methylamino)-6-oxo-1,4,5,6-tetrahydrobenzo[c][1,7]naphthyridine-3(2H)-carboxylate (Vaw) in an analogous manner as described above. The enantiomers were subsequently separated by preparative SFC: Method isocratic, Mobile phase (0.2% 7 M Methanolic Ammonia in acetonitrile:MeOH (1:1) v/v): CO₂—45:55. Column: Chiralpak-IE (30×250 mm), 5μ, flow rate: 110 g/min.

Enantiomer I (Compound 149): LCMS: m/z found 437.2/439.2 [M+H]⁺, RT=3.19 min, (Method A); ¹H NMR (400 MHz, DMSO-d₆) at 90° C.: δ 11.58 (br s, 1H), 8.58 (br s, 1H), 8.11-8.06 (m, 1H) 7.83 (dd, 1H), 7.51-7.47 (m, 1H), 7.37-7.30 (m, 2H), 5.33 (s, 1H), 3.75 (d, 1H), 3.60 (d, 1H), 3.09-3.02 (m, 2H), 2.80 (s, 3H), 2.75-2.66 (m, 1H); Chiral analytical SFC: RT=2.92 min, Column: Chiralpak IE-3 (4.6×150 mm) 3 μm, 40% methanol, Flow rate: 3 g/min.

Enantiomer II (Compound 150): LCMS: m/z found 437.2/439.2 [M+H]⁺, RT=3.19 min, (Method A); ¹H NMR (400 MHz, DMSO-d₆) at 90° C.: δ 11.57 (br s, 1H), 8.57 (br s, 1H), 8.11-8.06 (m, 1H) 7.83 (dd, 1H), 7.52-7.48 (m, 1H), 7.37-7.26 (m, 2H), 5.46 (s, 1H), 3.75 (d, 1H), 3.60 (d, 1H), 3.09-3.02 (m, 2H), 2.80 (s, 3H), 2.75-2.66 (m, 1H); Chiral analytical SFC: RT=5.18 min, Column: Chiralpak IE-3 (4.6×150 mm) 3 μm, 40% methanol, Flow rate: 3 g/min.

### 1-(8,9-Difluoro-6-oxo-1,2,3,4,5,6-hexahydrobenzo[c][1,7]naphthyridin-1-yl)-3-(3,4-difluorophenyl)-1-methylurea (Compounds 191 and 192)

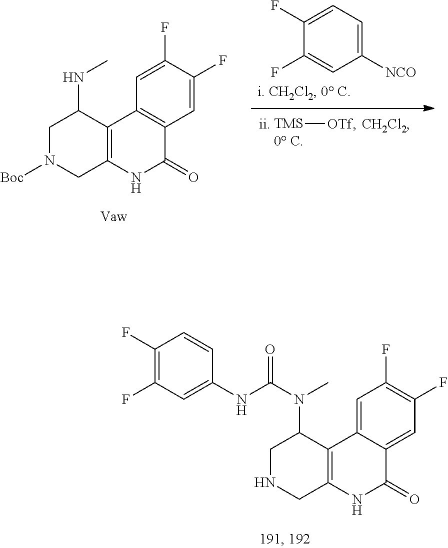

Racemic 1-(8,9-difluoro-6-oxo-1,2,3,4,5,6-hexahydrobenzo[c][1,7]naphthyridin-1-yl)-3-(3,4-difluorophenyl)-1-methylurea was synthesized from tert-butyl 8,9-difluoro-1-(methylamino)-6-oxo-1,4,5,6-tetrahydrobenzo[c][1,7]naphthyridine-3(2H)-carboxylate (Vaw) and 1,2-difluoro-4-isocyanatobenzene in an analogous manner as described above. The enantiomers were subsequently separated by preparative SFC: Method isocratic, Mobile phase (0.2% 7 M Methanolic Ammonia in Acetonitrile:MeOH (1:1) v/v): CO₂—50:50. Column: Chiralcel-IE (30×250 mm), 5μ, flow rate: 120 g/min.

Enantiomer I (Compound 191): LCMS: m/z found 421.2 [M+H]⁺, RT=4.47 min, (Method A); ¹H NMR (400 MHz, DMSO-d₆): δ 11.5 (brs, 1H), 8.59 (brs, 1H), 8.11-8.06 (t, 1H), 7.73-7.68 (m, 1H), 7.37-7.32 (m, 3H), 5.33 (s, 1H), 3.73 (d, 1H), 3.58 (d, 1H), 3.06 (s, 2H),), 2.80-2.66 (m, 4H); Chiral analytical SFC: RT=1.22 min, Column: Chiralcel IE-3 (4.6×150 mm) 3 μm, 50% (0.2% 7M Methanolic ammonia in ACN:MeOH (1:1)), Flow rate: 3 g/min.

Enantiomer II (Compound 192): LCMS: m/z found 421.2 [M+H]⁺, RT=4.47 min, min, (Method A); ¹H NMR (400 MHz, DMSO-d₆): δ 11.5 (brs, 1H), 8.59 (brs, 1H), 8.11-8.06 (t, 1H), 7.73-7.68 (m, 1H), 7.37-7.32 (m, 3H), 5.33 (s, 1H), 3.73 (d, 1H), 3.58 (d, 1H), 3.06 (s, 2H),), 2.80-2.66 (m, 4H); Chiral analytical SFC: RT=2.37 min, Column: Chiralcel IE-3 (4.6×150 mm) 3 μm, 50% (0.2% 7M Methanolic ammonia in ACN:MeOH (1:1)), Flow rate: 3 g/min.

### 3-(3-Cyano-4-fluorophenyl)-1-(8,9-difluoro-6-oxo-1,2,3,4,5,6-hexahydrobenzo[c][1,7]naphthyridin-1-yl)-1-methylurea (Compounds 175 and 176)

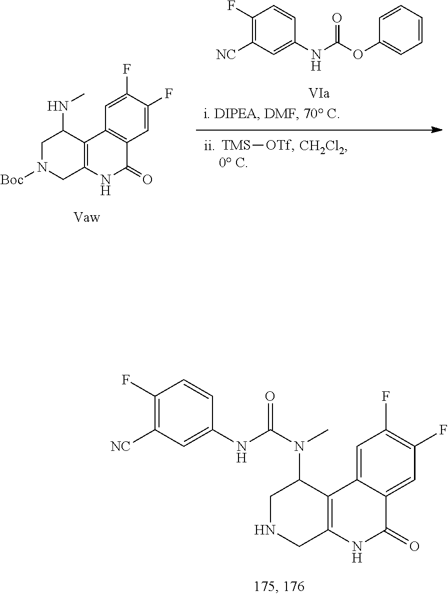 [SMILES: N#Cc1cc(NC(=O)Oc2ccccc2)ccc1F ; CNC1CN(C(=O)OC(C)(C)C)Cc2[nH]c(=O)c3cc(F)c(F)cc3c21 ; C[Si](C)(C)OS(=O)(=O)C(F)(F)F ; CN(C(=O)Nc1ccc(F)c(C#N)c1)C1CNCc2[nH]c(=O)c3cc(F)c(F)cc3c21]

Racemic 3-(3-cyano-4-fluorophenyl)-1-(8,9-difluoro-6-oxo-1,2,3,4,5,6-hexahydrobenzo[c][1,7]naphthyridin-1-yl)-1-methylurea was prepared from tert-butyl 8,9-difluoro-1-(methylamino)-6-oxo-1,4,5,6-tetrahydrobenzo[c][1,7]naphthyridine-3(2H)-carboxylate (Vaw) in an analogous manner as described above. The enantiomers were subsequently separated by preparative SFC: Method isocratic, Mobile phase (0.2% 7 M Methanolic Ammonia in Acetonitrile:MeOH (1:1) v/v): CO₂—45:55. Column: Chiralpak-IE (30×250 mm), 5μ, flow rate: 120 g/min.

Enantiomer I (Compound 175): LCMS: m/z found 428.2 [M+H]⁺, RT=3.19 min, (Method A); ¹H NMR (400 MHz, DMSO-d₆): δ 11.55 (br s, 1H), 8.76 (br s, 1H), 8.11-8.04 (m, 2H) 7.91-7.84 (m, 1H), 7.46 (t, 1H), 7.35-7.30 (m, 2H), 5.32 (s, 1H), 3.76 (d, 1H), 3.60 (d, 1H), 3.12-3.07 (m, 2H), 2.81 (s, 3H), 2.75-2.66 (m, 1H); Chiral analytical SFC: RT=2.84 min, Column: Chiralpak IE-3 (4.6×150 mm) 3 μm, 40% (0.2% 7 M Methanolic ammonia in Acetonitrile:MeOH (1:1) v/v), Flow rate: 3 g/min.

Enantiomer II (Compound 176): LCMS: m/z found 428.2 [M+H]⁺, RT=3.19 min, min, (Method A); ¹H NMR (400 MHz, DMSO-d₆): δ 11.55 (br s, 1H), 8.76 (br s, 1H), 8.11-8.04 (m, 2H) 7.91-7.84 (m, 1H), 7.46 (t, 1H), 7.35-7.30 (m, 2H), 5.32 (s, 1H), 3.76 (d, 1H), 3.60 (d, 1H), 3.12-3.07 (m, 2H), 2.81 (s, 3H), 2.75-2.66 (m, 1H); Chiral analytical SFC: RT=6.65 min, Column: Chiralpak IE-3 (4.6×150 mm) 3 μm, 40% (0.2% 7M Methanolic ammonia in Acetonitrile:MeOH (1:1) v/v), Flow rate: 3 g/min.

### 1-(8,9-Difluoro-6-oxo-1,2,3,4,5,6-hexahydrobenzo[c][1,7]naphthyridin-1-yl)-3-(3-(difluoromethyl)-4-fluorophenyl)-1-methylurea (Compounds 216 and 217)

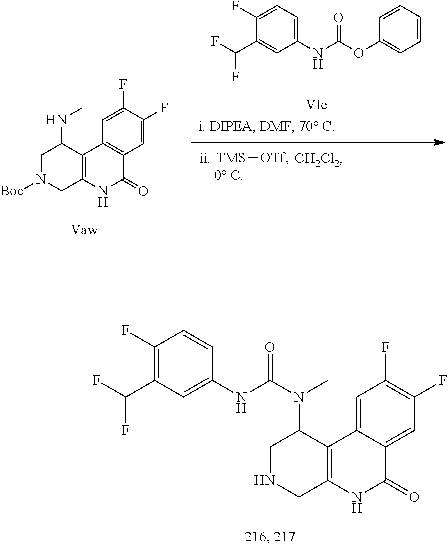 [SMILES: O=C(Nc1ccc(F)c(C(F)F)c1)Oc1ccccc1 ; CNC1CN(C(=O)OC(C)(C)C)Cc2[nH]c(=O)c3cc(F)c(F)cc3c21 ; C[Si](C)(C)OS(=O)(=O)C(F)(F)F ; CN(C(=O)Nc1ccc(F)c(C(F)F)c1)C1CNCc2[nH]c(=O)c3cc(F)c(F)cc3c21]

Racemic tert-butyl 1-(3-(3-(difluoromethyl)-4-fluorophenyl)-1-methylureido)-8,9-difluoro-6-oxo-1,2,5,6-tetrahydrobenzo[c][1,7]naphthyridine-3(4H)-carboxylate was prepared from tert-butyl 8,9-difluoro-1-(methylamino)-6-oxo-1,4,5,6-tetrahydrobenzo[c][1,7]naphthyridine-3(2H)-carboxylate (Vaw) and 3-(difluoromethyl)-4-fluorophenylcarbamate (VIe) in an analogous manner as described above. The enantiomers were subsequently separated by preparative SFC: Method isocratic, Mobile phase MeOH: CO₂— 30:70. Column: Chiralpak-OX-3 (30×250 mm), 5μ, flow rate: 100 g/min. Each enantiomer was converted to a single enantiomer of 1-(8,9-difluoro-6-oxo-1,2,3,4,5,6-hexahydrobenzo[c][1,7]naphthyridin-1-yl)-3-(3-(difluoromethyl)-4-fluorophenyl)-1-methylurea in an analogous manner as described above.

Enantiomer I (Compound 216): LCMS: m/z found 453.3 [M+H]⁺, RT=7.20 min, (Method A); ¹H NMR (400 MHz, DMSO-d₆): δ 11.57 (s, 1H), 8.63 (br s, 1H), 8.09 (t, 1H), 7.87 (d, 1H), 7.73-7.71 (m, 1H), 7.38-7.06 (m, 3H), 5.34 (br s, 1H) 3.75 (d, 1H), 3.60 (d, 1H), 3.09-3.02 (m, 2H), 2.80 (s, 3H), 2.73 (br s, 1H); Chiral analytical SFC: RT=5.00 min, Column: Chiralpak OX-3 (4.6×150 mm) 3 μm, 20% (0.5% of DEA in Methanol), Flow rate: 3 g/min.

Enantiomer II (Compound 217): LCMS: m/z found 453.3 [M+H]⁺, RT=7.20 min, min, (Method A); ¹H NMR (400 MHz, DMSO-d₆): δ 11.57 (s, 1H), 8.63 (br s, 1H), 8.09 (t, 1H), 7.87 (d, 1H), 7.73-7.71 (m, 1H), 7.38-7.06 (m, 3H), 5.34 (br s, 1H) 3.75 (d, 1H), 3.60 (d, 1H), 3.09-3.02 (m, 2H), 2.80 (s, 3H), 2.73 (br s, 1H); Chiral analytical SFC: RT=5.58 min, Column: Chiralpak OX-3 (4.6×150 mm) 3 μm, 20% (0.5% of DEA in Methanol), Flow rate: 3 g/min.

### N-(8,9-Difluoro-6-oxo-1,2,3,4,5,6-hexahydrobenzo[c][1,7]naphthyridin-1-yl)-N-methylisoindoline-2-carboxamide (Compounds 222 and 223)

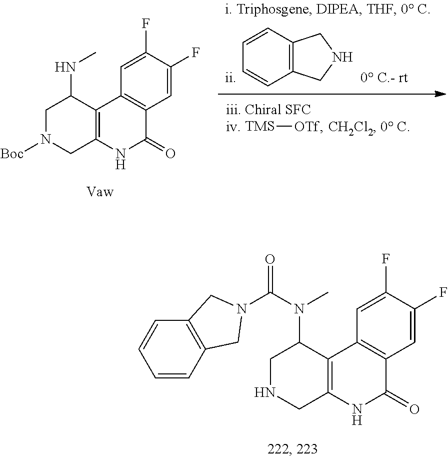

To a stirred solution of 150 mg (0.136 mmol, 1 eq) of racemic tert-butyl 8,9-difluoro-1-(methylamino)-6-oxo-1,4,5,6-tetrahydrobenzo[c][1,7]naphthyridine-3(2H)-carboxylate (Vaw) in 2 mL of THF was added 0.17 ml DIPEA (0.96 mmol, 2.3 eq) and 74 mg (0.82 mmol, 0.6 eq) triphosgene at 0° C. and the reaction mixture was stirred at the same temperature for 30 min. Isoindoline (50 mg, 0.136 mmol, 1 eq) was added and the reaction was continued at the same temperature for 4 h. The reaction mixture was poured into water (20 mL) and extracted with ethyl acetate (2×10 mL). Combined organic layers were washed with water (10 mL), dried over anhydrous sodium sulfate, and concentrated under reduced pressure. The product was purified by column chromatography (Silicagel, isochratic 60% ethyl acetate in petroleum ether) to afford 90 mg (0.17 mmol, 42% yield) of racemic tert-butyl 8,9-difluoro-1-(N-methylisoindoline-2-carboxamido)-6-oxo-1,4,5,6-tetrahydro benzo[c][1,7]naphthyridine-3(2H)-carboxylate as an off white solid. The enantiomers were subsequently separated by preparative SFC: Method isocratic, Mobile phase MeOH: CO₂ —30:70. Column: Chiralcel OD-3 (30×250 mm), 5μ, flow rate: 110 g/min. Each enantiomer was individually converted to the final product by treatment with trimethylsilyl trifluoromethanesulfonate in an analogous manner as described above.

Enantiomer I (Compound 223): LCMS: m/z found 411.2 [M+H]⁺, RT=2.96 min, (Method A); ¹H NMR (400 MHz, DMSO-d₆): δ 11.55 (bs, 1H), 8.08 (t, 1H), 7.63-7.58 (m, 1H), 7.33-7.26 (m, 4H), 5.11 (s, 1H), 4.79 (d, 2H), 4.68 (d, 2H), 3.75 (d, 1H), 3.60 (d, 1H), 3.20 (d, 1H), 3.10-3.06 (m, 1H), 2.79-2.67 (m, 4H); Chiral analytical SFC: RT=4.62 min, Column: Chiralcel OX-3 (4.6×150 mm) 3 μm, 40% (0.5% DEA in Methanol), Flow rate: 3 g/min.

Enantiomer II (Compound 222): LCMS: m/z found 411.2 [M+H]⁺, RT=2.96 min, min, (Method A); ¹H NMR (400 MHz, DMSO-d₆): δ 11.55 (bs, 1H), 8.08 (t, 1H), 7.63-7.58 (m, 1H), 7.33-7.26 (m, 4H), 5.11 (s, 1H), 4.79 (d, 2H), 4.68 (d, 2H), 3.75 (d, 1H), 3.60 (d, 1H), 3.20 (d, 1H), 3.10-3.06 (m, 1H), 2.79-2.63 (m, 4H); Chiral analytical SFC: RT=5.71 min, Column: Chiralcel OX-3 (4.6×150 mm) 3 μm, 40% (0.5% DEA in Methanol), Flow rate: 3 g/min.

### N-(8,9-Difluoro-6-oxo-1,2,3,4,5,6-hexahydrobenzo[c][1,7]naphthyridin-1-yl)-5-fluoro-N-methylisoindoline-2-carboxamide (Compounds 224 and 225)

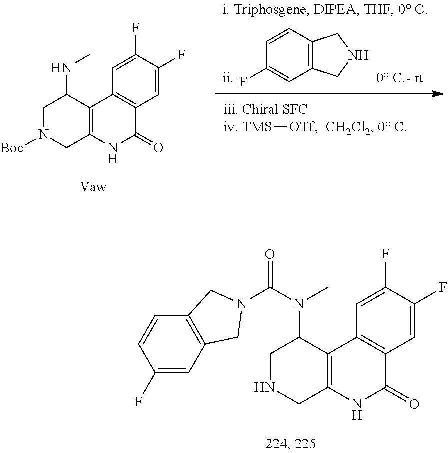

N-(8,9-Difluoro-6-oxo-1,2,3,4,5,6-hexahydrobenzo[c][1,7]naphthyridin-1-yl)-5-fluoro-N-methylisoindoline-2-carboxamide was prepared from tert-butyl 8,9-difluoro-1-(methylamino)-6-oxo-1,4,5,6-tetrahydrobenzo[c][1,7]naphthyridine-3(2H)-carboxylate (Vaw) and 5-fluoro isoindoline in an analogous manner as described above. The enantiomers of intermediate tert-butyl 8,9-difluoro-1-(5-fluoro-N-methylisoindoline-2-carboxamido)-6-oxo-1,4,5,6-tetrahydrobenzo[c][1,7]naphthyridine-3(2H)-carboxylate were separated by preparative SFC: Method isocratic, Mobile phase MeOH: CO₂—30:70. Column: Chiralcel OD-3 (30×250 mm), 5μ, flow rate: 110 g/min.

Enantiomer I (Compound 225): LCMS: m/z found 429.2 [M+H]⁺, RT=3.10 min, (Method A); ¹H NMR (400 MHz, DMSO-d₆): δ 11.55 (bs, 1H), 8.08 (t, 1H), 7.62-7.57 (m, 1H), 7.36-7.33 (m, 1H), 7.19-7.08 (m, 2H), 5.10 (s, 1H), 4.80-4.61 (m, 4H), 3.74 (d, 1H), 3.59 (d, 1H), 3.20 (d, 1H), 3.09 (d, 1H), 2.77 (m, 3H); Chiral analytical SFC: RT=3.88 min, Column: Chiralcel OX-3 (4.6×150 mm) 3 μm, 40% (0.5% DEA in Methanol), Flow rate: 3 g/min.

Enantiomer II (Compound 224): LCMS: m/z found 429.2 [M+H]⁺, RT=3.10 min, min, (Method A); ¹H NMR (400 MHz, DMSO-d₆): δ 11.55 (bs, 1H), 8.08 (t, 1H), 7.62-7.57 (m, 1H), 7.36-7.33 (m, 1H), 7.19-7.08 (m, 2H), 5.10 (s, 1H), 4.80-4.61 (m, 4H), 3.74 (d, 1H), 3.59 (d, 1H), 3.20 (d, 1H), 3.09 (d, 1H), 2.77 (m, 3H); Chiral analytical SFC: RT=4.85 min, Column: Chiralcel OX-3 (4.6×150 mm) 3 μm, 40% (0.5% DEA in Methanol), Flow rate: 3 g/min.

### 5-Chloro-N-(8,9-difluoro-6-oxo-1,2,3,4,5,6-hexahydrobenzo[c][1,7]naphthyridin-1-yl)-N-methylisoindoline-2-carboxamide (Compounds 226 and 227)

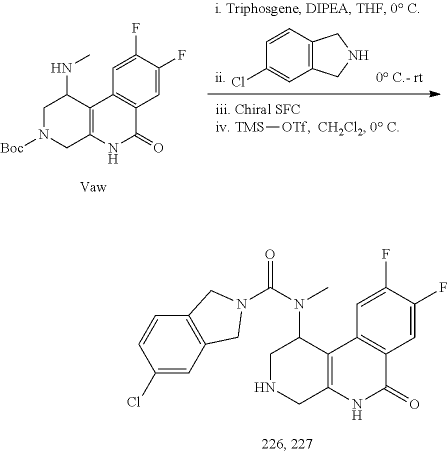

5-Chloro-N-(8,9-difluoro-6-oxo-1,2,3,4,5,6-hexahydrobenzo[c][1,7]naphthyridin-1-yl)-N-methylisoindoline-2-carboxamide was prepared from tert-butyl 8,9-difluoro-1-(methylamino)-6-oxo-1,4,5,6-tetrahydrobenzo[c][1,7]naphthyridine-3(2H)-carboxylate (Vaw) and 5-chloro-isoindoline in an analogous manner as described above. The enantiomers of intermediate tert-butyl-1-(5-chloro-N-methylisoindoline-2-carboxamido)-8,9-difluoro-6-oxo-1,4,5,6-tetrahydrobenzo[c][1,7]naphthyridine-3(2H)-carboxylate were separated by preparative SFC: Method isocratic, Mobile phase MeOH: CO₂—30:70. Column: Chiralcel OJ-3 (30×250 mm), 5μ, flow rate: 110 g/min.

Enantiomer I (Compound 226): LCMS: m/z found 445.2/447.2 [M+H]⁺, RT=3.43 min, (Method A); ¹H NMR (400 MHz, DMSO-d₆): δ 11.55 (bs, 1H), 8.08 (t, 1H), 7.62-7.57 (m, 1H), 7.42 (m, 1H), 7.34 (t, 2H), 5.10 (s, 1H), 4.80-4.64 (m, 4H), 3.74 (d, 1H), 3.59 (d, 1H), 3.18-3.06 (m, 2H), 2.77 (m, 3H); Chiral analytical SFC: RT=3.88 min, Column: Chiralcel OJ-3 (4.6×150 mm) 3 μm, 20% (0.5% DEA in Methanol), Flow rate: 3 g/min.

Enantiomer II (Compound 227): LCMS: m/z found 445.2/447.2 [M+H]⁺, RT=3.43 min, min, (Method A); ¹H NMR (400 MHz, DMSO-d₆): δ 11.55 (bs, 1H), 8.08 (t, 1H), 7.62-7.57 (m, 1H), 7.42 (m, 1H), 7.34 (t, 2H), 5.10 (s, 1H), 4.80-4.64 (m, 4H), 3.74 (d, 1H), 3.59 (d, 1H), 3.18-3.06 (m, 2H), 2.77 (m, 3H); Chiral analytical SFC: RT=4.85 min, Column: Chiralcel OJ-3 (4.6×150 mm) 3 μm, 20% (0.5% DEA in Methanol), Flow rate: 3 g/min.

### 5-Bromo-N-(8,9-difluoro-6-oxo-1,2,3,4,5,6-hexahydrobenzo[c][1,7]naphthyridin-1-yl)-N-methylisoindoline-2-carboxamide (Compounds 228 and 229)

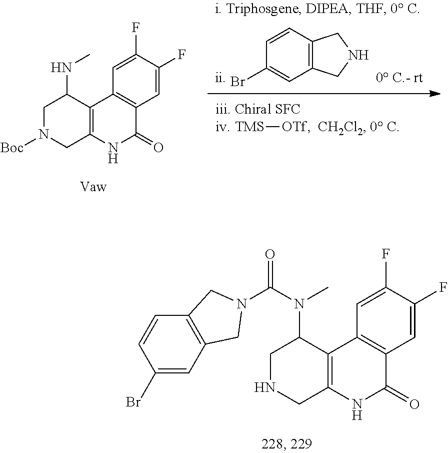

5-Bromo-N-(8,9-difluoro-6-oxo-1,2,3,4,5,6-hexahydrobenzo[c][1,7]naphthyridin-1-yl)-N-methylisoindoline-2-carboxamide was prepared from tert-butyl 8,9-difluoro-1-(methylamino)-6-oxo-1,4,5,6-tetrahydrobenzo[c][1,7]naphthyridine-3(2H)-carboxylate (Vaw) and 5-bromo-isoindoline in an analogous manner as described above. The enantiomers of intermediate tert-butyl-1-(5-bromo-N-methylisoindoline-2-carboxamido)-8,9-difluoro-6-oxo-1,4,5,6-tetrahydrobenzo[c][1,7]naphthyridine-3(2H)-carboxylate were separated by preparative SFC: Method isocratic, Mobile phase MeOH: CO₂—30:70. Column: Chiralcel OJ-3 (30×250 mm), 5μ, flow rate: 110 g/min.

Enantiomer I (Compound 228): LCMS: m/z found 491.1 [M+H]⁺, RT=3.55 min, (Method A); ¹H NMR (400 MHz, DMSO-d₆): δ 11.55 (bs, 1H), 8.08 (t, 1H), 7.62-7.56 (m, 2H), 7.46 (d, 1H), 7.29 (d, 1H), 5.10 (s, 1H), 4.80-4.62 (m, 4H), 3.74 (d, 1H), 3.59 (d, 1H), 3.21-3.08 (m, 2H), 2.77 (m, 3H), 2.61 (s, 1H); Chiral analytical SFC: RT=3.46 min, Column: Chiralcel OJ-3 (4.6×150 mm) 3 μm, 20% (0.5% DEA in Methanol), Flow rate: 3 g/min.

Enantiomer II (Compound 229): LCMS: m/z found 491.1 [M+H]⁺, RT=3.55 min, min, (Method A); ¹H NMR (400 MHz, DMSO-d₆): δ 11.55 (bs, 1H), 8.08 (t, 1H), 7.62-7.56 (m, 2H), 7.46 (d, 1H), 7.29 (d, 1H), 5.10 (s, 1H), 4.80-4.62 (m, 4H), 3.74 (d, 1H), 3.59 (d, 1H), 3.21-3.08 (m, 2H), 2.77 (m, 3H), 2.61 (s, 1H); Chiral analytical SFC: RT=5.42 min, Column: Chiralcel OJ-3 (4.6×150 mm) 3 μm, 20% (0.5% DEA in Methanol), Flow rate: 3 g/min.

### N-(8,9-Difluoro-6-oxo-1,2,3,4,5,6-hexahydrobenzo[c][1,7]naphthyridin-1-yl)-N-methyl-5-(trifluoromethyl)isoindoline-2-carboxamide (Compounds 231 and 232)

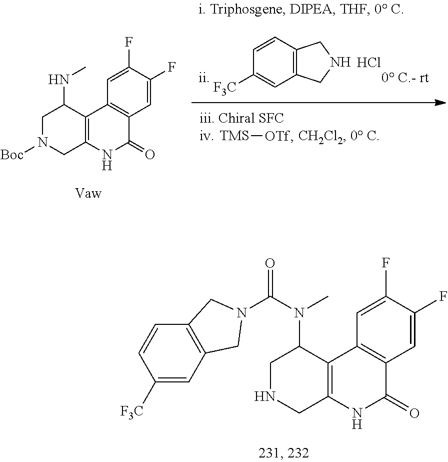 [SMILES: CNC1CN(C(=O)OC(C)(C)C)Cc2[nH]c(=O)c3cc(F)c(F)cc3c21 ; FC(F)(F)c1ccc2c(c1)CNC2 ; C[Si](C)(C)OS(=O)(=O)C(F)(F)F ; Cl ; CN(C(=O)N1Cc2ccc(C(F)(F)F)cc2C1)C1CNCc2[nH]c(=O)c3cc(F)c(F)cc3c21]

N-(8,9-Difluoro-6-oxo-1,2,3,4,5,6-hexahydrobenzo[c][1,7]naphthyridin-1-yl)-N-methyl-5-(trifluoromethyl)isoindoline-2-carboxamide was prepared from tert-butyl 8,9-difluoro-1-(methylamino)-6-oxo-1,4,5,6-tetrahydrobenzo[c][1,7]naphthyridine-3(2H)-carboxylate (Vaw) and 5-(trifluoromethyl)isoindoline hydrochloride in an analogous manner as described above. The enantiomers of intermediate tert-butyl 8,9-difluoro-1-(N-methyl-5-(trifluoromethyl) isoindoline-2-carboxamido)-6-oxo-1,4,5,6-tetrahydrobenzo[c][1,7]naphthyridine-3(2H)-carboxylate were separated by preparative SFC: Method isocratic, Mobile phase MeOH: CO₂—30:70. Column: Chiralcel OJ-3 (30×250 mm), 5μ, flow rate: 110 g/min.

Enantiomer I (Compound 231): LCMS: m/z found 479.1 [M+H]⁺, RT=3.64 min, (Method A); ¹H NMR (400 MHz, DMSO-d₆): δ 11.56 (bs, 1H), 8.09 (t, 1H), 7.73 (s, 1H), 7.65-7.54 (m, 3H), 5.11 (s, 1H), 4.86 (d, 2H), 4.76 (d, 2H), 3.75 (d, 1H), 3.60 (d, 1H), 3.23-3.16 (m, 1H), 3.1-3.06 (m, 1H), 2.79-2.63 (m, 4H); Chiral analytical SFC: RT=1.30 min, Column: Chiralcel OJ-3 (4.6×150 mm) 3 μm, 20% (0.5% DEA in Methanol), Flow rate: 3 g/min.

Enantiomer II (Compound 232): LCMS: m/z found 479.1 [M+H]⁺, RT=3.64 min, min, (Method A); ¹H NMR (400 MHz, DMSO-d₆): δ 11.56 (bs, 1H), 8.09 (t, 1H), 7.73 (s, 1H), 7.65-7.54 (m, 3H), 5.11 (s, 1H), 4.86 (d, 2H), 4.76 (d, 2H), 3.75 (d, 1H), 3.60 (d, 1H), 3.23-3.16 (m, 1H), 3.1-3.06 (m, 1H), 2.79-2.63 (m, 4H); Chiral analytical SFC: RT=1.69 min, Column: Chiralcel OJ-3 (4.6×150 mm) 3 μm, 20% (0.5% DEA in Methanol), Flow rate: 3 g/min.

### 3-(2-((tert-Butyldimethylsilyl)oxy)ethyl)-8,9-difluoro-1-(methylamino)-1,3,4,5-tetrahydrobenzo[c][1,7]naphthyridin-6(2H)-one (Vax)

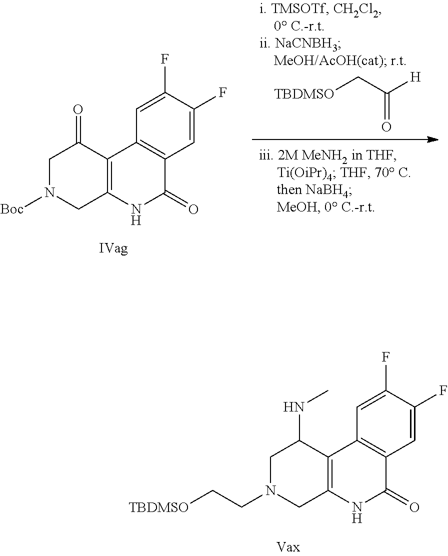 [SMILES: CC(C)(C)OC(=O)N1CC(=O)c2c([nH]c(=O)c3cc(F)c(F)cc23)C1 ; CC(C)(C)[Si](C)(C)OCC=O ; CNC1CN(CCO[Si](C)(C)C(C)(C)C)Cc2[nH]c(=O)c3cc(F)c(F)cc3c21]

Racemic 3-(2-((Tert-butyldimethylsilyl)oxy)ethyl)-8,9-difluoro-1-(methylamino)-1,3,4,5-tetrahydrobenzo[c][1,7]naphthyridin-6(2H)-one was prepared from tert-butyl 8,9-difluoro-1,6-dioxo-1,4,5,6-tetrahydrobenzo[c][1,7]naphthyridine-3(2H)-carboxylate (IVag), 2-((tert-butyldimethylsilyl)oxy)acetaldehyde, and methylamine by a procedure similar to the one described above for Vat. LCMS: m/z found 422.5 [M−H]⁻.

### 3-(3-Chloro-4-fluorophenyl)-1-(8,9-difluoro-3-(2-hydroxyethyl)-6-oxo-1,2,3,4,5,6-hexahydrobenzo[c][1,7]naphthyridin-1-yl)-1-methylurea (Compounds 179 and 180)

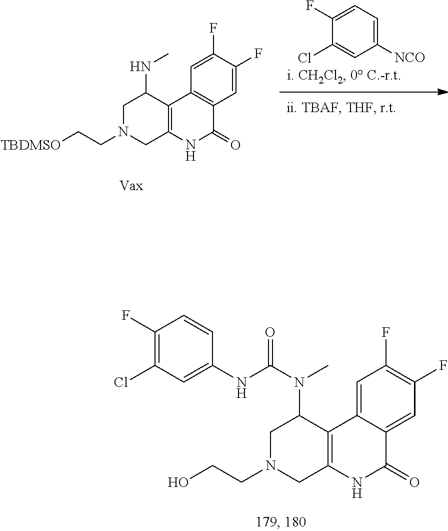

Step i. To a stirred solution of crude 3-(2-((tert-butyldimethylsilyl)oxy)ethyl)-8,9-difluoro-1-(methylamino)-1,3,4,5-tetrahydrobenzo[c][1,7]naphthyridin-6(2H)-one (Vax, 271 mg, 0.64 mmol, 1.0 eq) in 5 mL of dichloromethane at 0° C., 2-chloro-1-fluoro-4-isocyanatobenzene (35 μL, 0.288 mmol, 0.45 eq based on purity of Vax) was added and the resulting reaction mixture was stirred at room temperature for 1 h. The mixture was then diluted with water (15 mL) and extracted with 10% MeOH in dichloromethane (2×30 mL). The combined organic layer was washed with brine (30 mL), dried over anhydrous Na₂SO₄ and concentrated under reduced pressure. The crude product was purified by flash chromatography (Silicagel, 4% MeOH in dichloromethane, isochratic) to afford 113 mg (0.19 mmol, 69%) of 1-(3-(2-((tert-butyldimethylsilyl)oxy)ethyl)-8,9-difluoro-6-oxo-1,2,3,4,5,6-hexahydrobenzo[c][1,7]naphthyridin-1-yl)-3-(3-chloro-4-fluorophenyl)-1-methylurea as a brown resin. LCMS m/z found 595.5 [M+H]⁺.

Step ii. To a stirred solution 113 mg (0.19 mmol, 1.0 eq) of 1-(3-(2-((tert-butyldimethylsilyl)oxy)ethyl)-8,9-difluoro-6-oxo-1,2,3,4,5,6-hexahydrobenzo[c][1,7]naphthyridin-1-yl)-3-(3-chloro-4-fluorophenyl)-1-methylurea in 3 mL of THF at 0° C., TBAF (380 μL, 0.38 mmol, 2.0 eq) was added and the reaction was continued at room temperature for 12 h. The reaction was then quenched with MeOH (0.6 mL) and the organic volatiles were evaporated under reduced pressure. The residue was diluted with water (15 mL) and extracted with ethyl acetate (2×30 mL). The combined organic layer was washed with brine (30 mL), dried over anhydrous Na₂SO₄ and concentrated under reduced pressure. The resulting product was purified by flash chromatography (Silicagel, using 4.8% MeOH in dichloromethane, isocratic) to afford 3-(3-chloro-4-fluorophenyl)-1-(8,9-difluoro-3-(2-hydroxyethyl)-6-oxo-1,2,3,4,5,6-hexahydrobenzo[c][1,7]naphthyridin-1-yl)-1-methylurea (31 mg, 0.064 mmol, 33%). The enantiomers were subsequently separated by preparative SFC: Method isocratic, Mobile phase MeOH:CO₂ —40:60. Column: Chiralpak-IC (30×250 mm), 5μ, flow rate: 100 g/min.

Enantiomer I (Compound 179): LCMS: m/z found 481.1/483.2 [M+H]⁺, RT=4.03 min, (Method A); ¹H NMR (400 MHz, DMSO-d₆): δ 11.62 (br s, 1H), 8.55 (br s, 1H), 8.11-8.06 (m, 1H) 7.84 (dd, 1H), 7.58-7.48 (m, 1H), 7.41-7.30 (m, 2H), 5.47 (s, 1H), 4.53 (t, 1H), 3.78 (d, 1H), 3.51-3.58 (m, 2H), 3.17 (d, 1H), 3.02 (d, 1H), 2.83 (s, 3H), 2.73-2.67 (m, 1H), 2.59-2.51 (m, 2H); Chiral analytical SFC: RT=1.25 min, Column: Chiralpak IC-3 (4.6×150 mm) 3 μm, 40% Methanol, Flow rate: 3 g/min.

Enantiomer II (Compound 180): LCMS: m/z found 481.1/483.2 [M+H]⁺, RT=4.03 min, (Method A); ¹H NMR (400 MHz, DMSO-d₆): δ 11.62 (br s, 1H), 8.55 (br s, 1H), 8.11-8.06 (m, 1H) 7.84 (dd, 1H), 7.58-7.48 (m, 1H), 7.41-7.30 (m, 2H), 5.47 (s, 1H), 4.53 (t, 1H), 3.78 (d, 1H), 3.51-3.58 (m, 2H), 3.17 (d, 1H), 3.02 (d, 1H), 2.83 (s, 3H), 2.73-2.67 (m, 1H), 2.59-2.51 (m, 2H); Chiral analytical SFC: RT=1.83 min, Column: Chiralpak IC-3 (4.6×150 mm) 3 μm, 40% Methanol, Flow rate: 3 g/min.

### 3-(3-Chloro-4-fluorophenyl)-1-(8-fluoro-3-(2-hydroxyethyl)-6-oxo-1,2,3,4,5,6-hexahydrobenzo[c][1,7]naphthyridin-1-yl)-1-methylurea (Compounds 169 and 170)

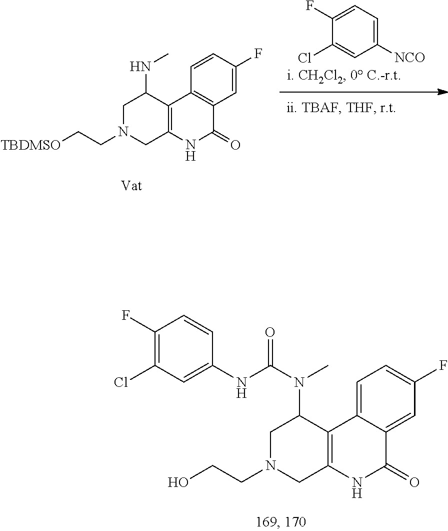

Racemic 3-(3-chloro-4-fluorophenyl)-1-(8-fluoro-3-(2-hydroxyethyl)-6-oxo-1,2,3,4,5,6-hexahydrobenzo[c][1,7]naphthyridin-1-yl)-1-methylurea was synthesized from 3-(2-((tert-butyldimethylsilyl)oxy)ethyl)-8-fluoro-1-(methylamino)-1,3,4,5-tetrahydrobenzo[c][1,7]naphthyridin-6(2H)-one (Vat) in an analogous manner as described above. The enantiomers were subsequently separated by preparative SFC: Method isocratic, Mobile phase MeOH:CO₂—40:60. Column: Chiralpak-IC (30×250 mm), 5μ, flow rate: 100 g/min.

Enantiomer I (Compound 169): LCMS: m/z found 463.2/465.2 [M+H]⁺, RT=3.30 min, (Method A); ¹H NMR (400 MHz, DMSO-d₆): δ 11.52 (br s, 1H), 8.55 (br s, 1H), 7.88-7.86 (m, 2H) 7.64 (dd, 1H), 7.58-7.48 (m, 2H), 7.41-7.30 (t, 1H), 5.5 (s, 1H), 4.53 (t, 1H), 3.88 (d, 1H), 3.53-3.58 (m, 2H), 3.17 (d, 1H), 3.02 (d, 1H), 2.83 (s, 3H), 2.73-2.67 (m, 1H), 2.59-2.51 (m, 2H); Chiral analytical SFC: RT=2.50 min, Column: Chiralpak IC-3 (4.6×150 mm) 3 μm, 40% Methanol, Flow rate: 3 g/min.

Enantiomer II (Compound 170): LCMS: m/z found 463.2/465.2 [M+H]⁺, RT=3.30 min, (Method A); ¹H NMR (400 MHz, DMSO-d₆): δ 11.52 (br s, 1H), 8.55 (br s, 1H), 7.88-7.86 (m, 2H) 7.64 (dd, 1H), 7.58-7.48 (m, 2H), 7.41-7.30 (t, 1H), 5.5 (s, 1H), 4.53 (t, 1H), 3.88 (d, 1H), 3.53-3.58 (m, 2H), 3.17 (d, 1H), 3.02 (d, 1H), 2.83 (s, 3H), 2.73-2.67 (m, 1H), 2.59-2.51 (m, 2H); Chiral analytical SFC: RT=3.65 min, Column: Chiralpak IC-3 (4.6×150 mm) 3 μm, 40% Methanol, Flow rate: 3 g/min.

### 3-(3-Cyano-4-fluorophenyl)-1-(8,9-difluoro-3-(2-hydroxyethyl)-6-oxo-1,2,3,4,5,6-hexahydrobenzo[c][1,7]naphthyridin-1-yl)-1-methylurea (Compounds 181 and 182

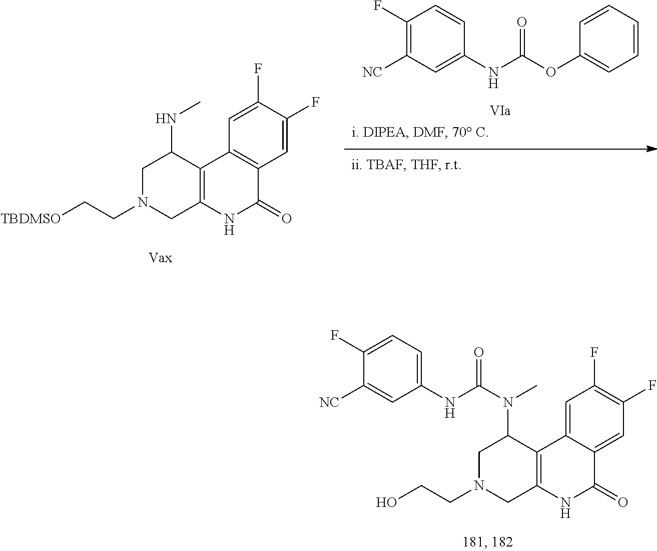 [SMILES: CNC1CN(CCO[Si](C)(C)C(C)(C)C)Cc2[nH]c(=O)c3cc(F)c(F)cc3c21 ; N#Cc1cc(NC(=O)Oc2ccccc2)ccc1F ; CN(C(=O)Nc1ccc(F)c(C#N)c1)C1CN(CCO)Cc2[nH]c(=O)c3cc(F)c(F)cc3c21]

Racemic 3-(3-cyano-4-fluorophenyl)-1-(8,9-difluoro-3-(2-hydroxyethyl)-6-oxo-1,2,3,4,5,6-hexahydrobenzo[c][1,7]naphthyridin-1-yl)-1-methylurea was synthesized from 3-(2-((tert-butyldimethylsilyl)oxy)ethyl)-8,9-difluoro-1-(methylamino)-1,3,4,5-tetrahydrobenzo[c][1,7]naphthyridin-6(2H)-one (Vax) in an analogous manner as described above. The enantiomers were subsequently separated by preparative SFC: Method isocratic, Mobile phase MeOH:CO₂—30:70. Column: Chiralpak-IC (30×250 mm), 5μ, flow rate: 100 g/min.

Enantiomer I (Compound 181): LCMS: m/z found 472.2 [M+H]⁺, RT=3.63 min, (Method A); ¹H NMR (400 MHz, DMSO-d₆): δ 11.63 (br s, 1H), 8.74 (br s, 1H), 8.11-8.06 (m, 2H) 7.86-7.83 (m, 1H), 7.46 (t, 1H), 7.39-7.34 (m, 1H), 5.47 (s, 1H), 4.53 (s, 1H), 3.78 (d, 1H), 3.56 (br s, 2H), 3.20 (d, 1H), 3.03 (dd, 1H), 2.84 (s, 3H), 2.73-2.67 (m, 1H), 2.56-2.49 (m, 2H); Chiral analytical SFC: RT=2.50 min, Column: Chiralpak IC-3 (4.6×150 mm) 3 μm, 40% Methanol, Flow rate: 3 g/min.

Enantiomer II (Compound 182): LCMS: m/z found 472.2 [M+H]⁺, RT=3.63 min, min, (Method A); ¹H NMR (400 MHz, DMSO-d₆): δ 11.63 (br s, 1H), 8.74 (br s, 1H), 8.11-8.06 (m, 2H) 7.86-7.83 (m, 1H), 7.46 (t, 1H), 7.39-7.34 (m, 1H), 5.47 (s, 1H), 4.53 (s, 1H), 3.78 (d, 1H), 3.56 (br s, 2H), 3.20 (d, 1H), 3.03 (dd, 1H), 2.84 (s, 3H), 2.73-2.67 (m, 1H), 2.56-2.49 (m, 2H); Chiral analytical SFC: RT=3.34 min, Column: Chiralpak IC-3 (4.6×150 mm) 3 μm, 40% Methanol, Flow rate: 3 g/min.

### 8,9-Difluoro-3-methyl-1-(methylamino)-1,3,4,5-tetrahydrobenzo[c][1,7]naphthyridin-6(2H)-one (Vay)

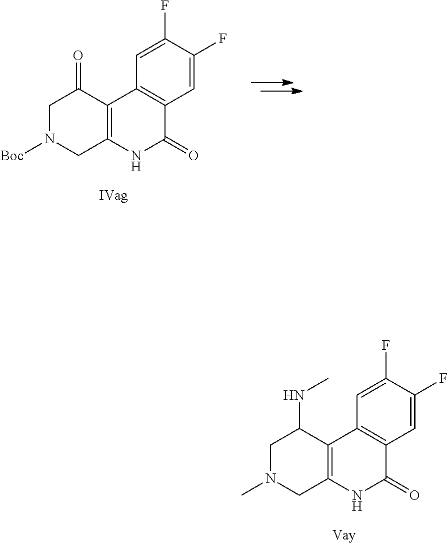 [SMILES: CC(C)(C)OC(=O)N1CC(=O)c2c([nH]c(=O)c3cc(F)c(F)cc23)C1 ; CNC1CN(C)Cc2[nH]c(=O)c3cc(F)c(F)cc3c21]

Racemic 8,9-difluoro-3-methyl-1-(methylamino)-1,3,4,5-tetrahydrobenzo[c][1,7]naphthyridin-6(2H)-one was synthesized in an analogous manner as described above for Vau, from tert-butyl 8,9-difluoro-1,6-dioxo-1,4,5,6-tetrahydrobenzo[c][1,7]naphthyridine-3(2H)-carboxylate (IVag), formaldehyde, and methylamine.

### 3-(3-Chloro-4-fluorophenyl)-1-(8,9-difluoro-3-methyl-6-oxo-1,2,3,4,5,6-hexahydrobenzo[c][1,7]naphthyridin-1-yl)-1-methylurea (Compounds 167 and 168)

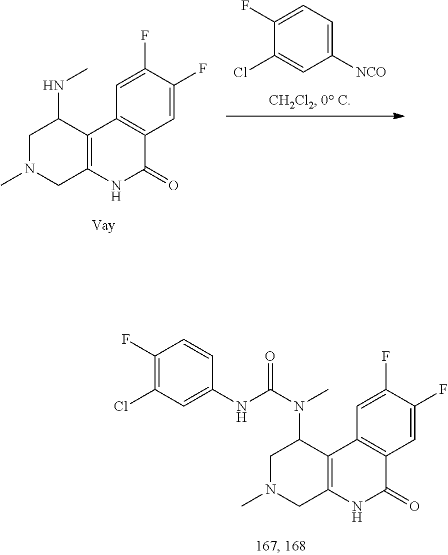

Racemic 3-(3-chloro-4-fluorophenyl)-1-(8,9-difluoro-3-methyl-6-oxo-1,2,3,4,5,6-hexahydrobenzo[c][1,7]naphthyridin-1-yl)-1-methylurea was synthesized from 8,9-difluoro-3-methyl-1-(methylamino)-1,3,4,5-tetrahydrobenzo[c][1,7]naphthyridin-6(2H)-one (Vay) in an analogous manner as described above. The enantiomers were subsequently separated by preparative SFC: Method isocratic, Mobile phase MeOH:CO₂—40:60. Column: Chiralpak-IC (30×250 mm), 5μ, flow rate: 100 g/min.

Enantiomer I (Compound 167): LCMS: m/z found 451.2/453.2 [M+H]⁺, RT=3.27 min, (Method A); ¹H NMR (400 MHz, DMSO-d₆): δ 11.66 (br s, 1H), 8.54 (br s, 1H), 8.09 (dd, 1H) 7.83 (dd, 1H), 7.51-7.47 (m, 1H), 7.38-7.30 (m, 2H), 5.49 (br s, 1H), 3.66 (d, 1H), 3.00 (d, 1H), 2.90 (d, 1H), 2.799 (s, 3H), 2.61 (dd, 1H), 2.33 (s, 3H); Chiral analytical SFC: RT=1.71 min, Column: Chiralpak IC-3 (4.6×150 mm) 3 μm, 30% (0.5% DEA in methanol), Flow rate: 3 g/min.

Enantiomer II (Compound 168): LCMS: m/z found 451.2/453.2 [M+H]⁺, RT=3.27 min, (Method A); ¹H NMR (400 MHz, DMSO-d₆): δ 11.66 (br s, 1H), 8.54 (br s, 1H), 8.09 (dd, 1H) 7.83 (dd, 1H), 7.51-7.47 (m, 1H), 7.38-7.30 (m, 2H), 5.49 (br s, 1H), 3.66 (d, 1H), 3.00 (d, 1H), 2.90 (d, 1H), 2.799 (s, 3H), 2.61 (dd, 1H), 2.33 (s, 3H); Chiral analytical SFC: RT=3.02 min, Column: Chiralpak IC-3 (4.6×150 mm) 3 μm, 30% (0.5% DEA in methanol), Flow rate: 3 g/min.

### 3-(3-Cyano-4-fluorophenyl)-1-(8,9-difluoro-3-methyl-6-oxo-1,2,3,4,5,6-hexahydrobenzo[c][1,7]naphthyridin-1-yl)-1-methylurea (Compounds 173 and 174)

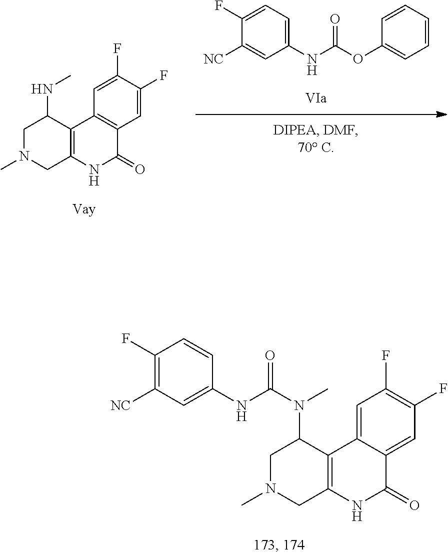 [SMILES: CNC1CN(C)Cc2[nH]c(=O)c3cc(F)c(F)cc3c21 ; N#Cc1cc(NC(=O)Oc2ccccc2)ccc1F ; CN1Cc2[nH]c(=O)c3cc(F)c(F)cc3c2C(N(C)C(=O)Nc2ccc(F)c(C#N)c2)C1]

Racemic 3-(3-cyano-4-fluorophenyl)-1-(8,9-difluoro-3-methyl-6-oxo-1,2,3,4,5,6-hexahydrobenzo[c][1,7]naphthyridin-1-yl)-1-methylurea was synthesized from 8,9-difluoro-3-methyl-1-(methylamino)-1,3,4,5-tetrahydrobenzo[c][1,7]naphthyridin-6(2H)-one (Vay) in an analogous manner as described above. The enantiomers were subsequently separated by preparative SFC: Method isocratic, Mobile phase (0.2% 7 M Methanolic Ammonia in Acetonitrile:MeOH (1:1) v/v):CO₂—25:75. Column: Chiralpak-IC (30×250 mm), 5μ, flow rate: 100 g/min.

Enantiomer I (Compound 173): LCMS: m/z found 442.2 [M+H]⁺, RT=3.18 min, (Method A); ¹H NMR (400 MHz, DMSO-d₆): δ 11.66 (br s, 1H), 8.72 (br s, 1H), 8.12-8.04 (m, 2H) 7.92-7.85 (m, 1H), 7.46 (t, 1H), 7.35 (dd, 1H), 5.49 (s, 1H), 3.67 (d, 1H), 3.00 (d, 1H), 2.95 (d, 1H), 2.81 (s, 3H), 2.62-2.58 (m, 1H), 2.33 (s, 3H); Chiral analytical SFC: RT=3.09 min, Column: Chiralpak IC-3 (4.6×150 mm) 3 μm, 25% (0.2% DEA in methanol), Flow rate: 3 g/min.

Enantiomer II (Compound 174): LCMS: m/z found 442.2 [M+H]⁺, RT=3.18 min, min, (Method A); ¹H NMR (400 MHz, DMSO-d₆): δ 11.66 (br s, 1H), 8.72 (br s, 1H), 8.12-8.04 (m, 2H) 7.92-7.85 (m, 1H), 7.46 (t, 1H), 7.35 (dd, 1H), 5.49 (s, 1H), 3.67 (d, 1H), 3.00 (d, 1H), 2.95 (d, 1H), 2.81 (s, 3H), 2.62-2.58 (m, 1H), 2.33 (s, 3H); Chiral analytical SFC: RT=4.41 min, Column: Chiralpak IC-3 (4.6×150 mm) 3 μm, 25% (0.2% DEA in methanol), Flow rate: 3 g/min.

### 3-Acetyl-8,9-difluoro-1-(methylamino)-1,3,4,5-tetrahydrobenzo[c][1,7]naphthyridin-6(2H)-one (Vaz)

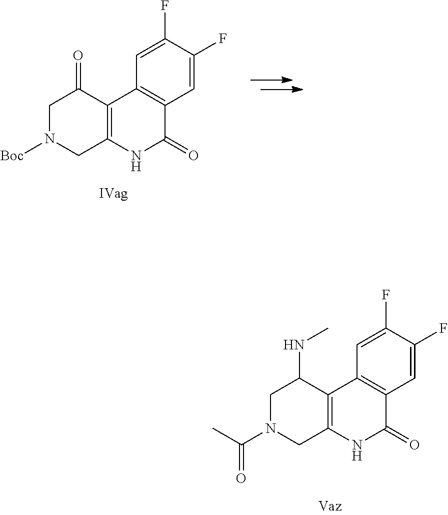 [SMILES: CC(C)(C)OC(=O)N1CC(=O)c2c([nH]c(=O)c3cc(F)c(F)cc23)C1 ; CNC1CN(C(C)=O)Cc2[nH]c(=O)c3cc(F)c(F)cc3c21]

Racemic 3-acetyl-8,9-difluoro-1-(methylamino)-1,3,4,5-tetrahydrobenzo[c][1,7]naphthyridin-6(2H)-one was synthesized in an analogous manner as described above for Vav, from tert-butyl 8,9-difluoro-1,6-dioxo-1,4,5,6-tetrahydrobenzo[c][1,7]naphthyridine-3(2H)-carboxylate (IVag), acetic anhydride, and methylamine. LCMS: m/z found 308.29 [M−H]⁺.

### 1-(3-Acetyl-8,9-difluoro-6-oxo-1,2,3,4,5,6-hexahydrobenzo[c][1,7]naphthyridin-1-yl)-3-(3-chloro-4-fluorophenyl)-1-methylurea (Compounds 159 and 160)

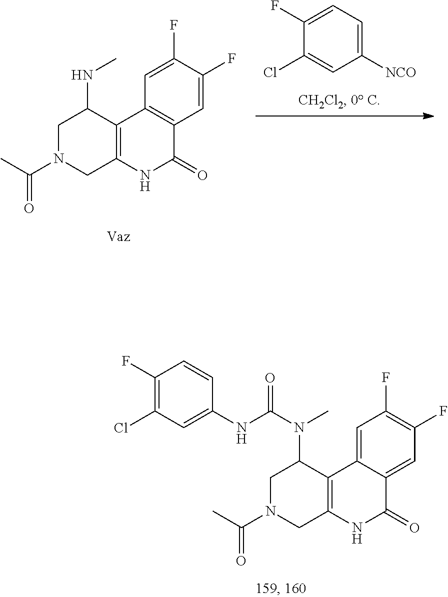

Racemic 1-(3-acetyl-8,9-difluoro-6-oxo-1,2,3,4,5,6-hexahydrobenzo[c][1,7]naphthyridin-1-yl)-3-(3-chloro-4-fluorophenyl)-1-methylurea was synthesized from 3-acetyl-8,9-difluoro-1-(methylamino)-1,3,4,5-tetrahydrobenzo[c][1,7]naphthyridin-6(2H)-one (Vaz) in an analogous manner as described above. The enantiomers were subsequently separated by preparative SFC: Method isocratic, Mobile phase MeOH:CO₂—40:60. Column: Chiralpak-IC (30×250 mm), 5μ, flow rate: 100 g/min.

Enantiomer I (Compound 159): LCMS: m/z found 479.2 [M+H]⁺, RT=4.09 min, (Method A); ¹H NMR (400 MHz, DMSO-d₆): δ 11.78 (br s, 1H), 8.58 (br s, 1H), 8.13-8.07 (m, 1H) 7.87-7.85 (m, 1H), 7.54-7.51 (m, 1H), 7.44-7.32 (m, 2H), 5.53 (s, 1H), 5.10 (d, 1H), 4.78 (d, 1H), 4.60-4.37 (m, 1H), 3.64 (dd, 1H), 2.61 (s, 3H), 2.11 (m, 1H); Chiral analytical SFC: RT=2.13 min, Column: Chiralpak IC-3 (4.6×150 mm) 3 μm, 40% methanol, Flow rate: 3 g/min.

Enantiomer II (Compound 160): LCMS: m/z found 479.2 [M+H]⁺, RT=4.09 min, (Method A); ¹H NMR (400 MHz, DMSO-d₆): δ 11.78 (br s, 1H), 8.58 (br s, 1H), 8.13-8.07 (m, 1H) 7.87-7.85 (m, 1H), 7.54-7.51 (m, 1H), 7.44-7.32 (m, 2H), 5.53 (s, 1H), 5.10 (d, 1H), 4.78 (d, 1H), 4.60-4.37 (m, 1H), 3.64 (dd, 1H), 2.61 (s, 3H), 2.11 (m, 1H); Chiral analytical SFC: RT=3.41 min, Column: Chiralpak IC-3 (4.6×150 mm) 3 μm, 40% methanol, Flow rate: 3 g/min.

### 1-(3-Acetyl-8,9-difluoro-6-oxo-1,2,3,4,5,6-hexahydrobenzo[c][1,7]naphthyridin-1-yl)-3-(3-cyano-4-fluorophenyl)-1-methylurea (Compounds 177 and 178)

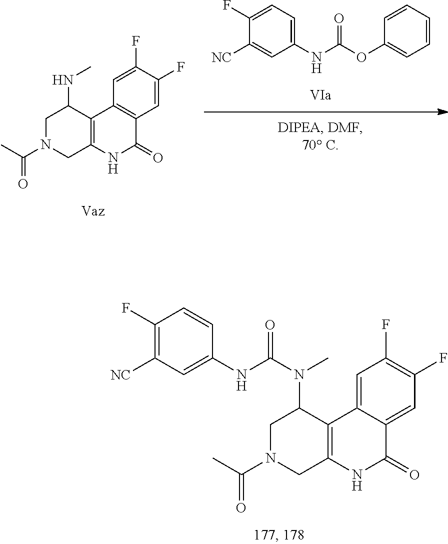 [SMILES: CNC1CN(C(C)=O)Cc2[nH]c(=O)c3cc(F)c(F)cc3c21 ; N#Cc1cc(NC(=O)Oc2ccccc2)ccc1F ; CC(=O)N1Cc2[nH]c(=O)c3cc(F)c(F)cc3c2C(N(C)C(=O)Nc2ccc(F)c(C#N)c2)C1]

Racemic 1-(3-acetyl-8,9-difluoro-6-oxo-1,2,3,4,5,6-hexahydrobenzo[c][1,7]naphthyridin-1-yl)-3-(3-cyano-4-fluorophenyl)-1-methylurea was synthesized from 3-acetyl-8,9-difluoro-1-(methylamino)-1,3,4,5-tetrahydrobenzo[c][1,7]naphthyridin-6(2H)-one (Vaz) in an analogous manner as described above. The enantiomers were subsequently separated by preparative SFC: Method isocratic, Mobile phase MeOH:CO₂—40:60. Column: Chiralpak-IC (30×250 mm), 5μ, flow rate: 100 g/min.

Enantiomer I (Compound 177): LCMS: m/z found 470.2 [M+H]⁺, RT=4.53 min, (Method A); ¹H NMR (400 MHz, DMSO-d₆): δ 11.78 (br s, 1H), 8.79-8.70 (m, 1H), 8.14-8.06 (m, 2H) 7.92-7.89 (m, 1H), 7.50-7.34 (m, 2H), 7.37-7.30 (m, 2H), 5.58 (s, 1H), 5.06 (d, 1H), 4.73 (d, 1H), 4.35 (d, 1H), 3.59 (d, 1H), 2.63 (s, 3H), 2.11 (s, 3H); Chiral analytical SFC: RT=2.47 min, Column: Chiralpak IC-3 (4.6×150 mm) 3 μm, 40% methanol, Flow rate: 3 g/min.

Enantiomer II (Compound 178): LCMS: m/z found 470.2 [M+H]⁺, RT=4.53 min, (Method A); ¹H NMR (400 MHz, DMSO-d₆): δ 11.78 (br s, 1H), 8.79-8.70 (m, 1H), 8.14-8.06 (m, 2H) 7.92-7.89 (m, 1H), 7.50-7.34 (m, 2H), 7.37-7.30 (m, 2H), 5.58 (s, 1H), 5.06 (d, 1H), 4.73 (d, 1H), 4.35 (d, 1H), 3.59 (d, 1H), 2.63 (s, 3H), 2.11 (s, 3H); Chiral analytical SFC: RT=3.66 min, Column: Chiralpak IC-3 (4.6×150 mm) 3 μm, 40% methanol, Flow rate: 3 g/min.

### 8,9-Difluoro-2H-thiopyrano[3,4-c]isoquinoline-1,6(4H,5H)-dione (IVah)

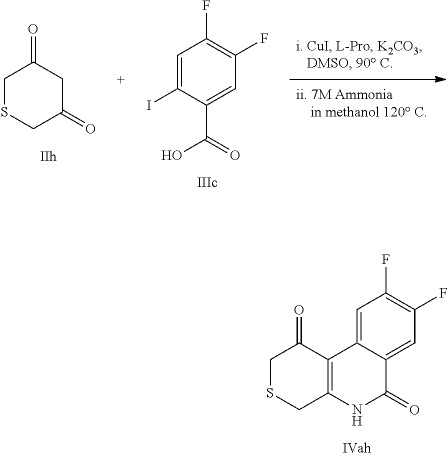 [SMILES: O=C1CSCC(=O)C1 ; O=C(O)c1cc(F)c(F)cc1I ; O=C1CSCc2[nH]c(=O)c3cc(F)c(F)cc3c21]

Step i: A mixture of 5.0 g (17.6 mmol, 1.0 eq) of 4,5-difluoro-2-iodobenzoic acid (IIIc), 2.74 g (21.12 mmol, 1.2 eq) of 2H-thiopyran-3,5(4H,6H)-dione (IIh), 9.7 g (70.4 mmol, 4.0 eq) of potassium carbonate, 0.41 g (3.5 mmol, 0.2 eq) of L-proline and 0.33 g (1.17 mmol, 0.1 eq) of copper(I)iodide in 30 mL of dry DMSO under a nitrogen atmosphere was stirred at 110° C. for 16 h (Note: Reaction was performed on 4×5 g scale in parallel). On cooling to room temperature, the reaction mixtures were combined and diluted with cold water (100 mL) and acidified with 2 M HCl solution (30 mL). The resulting suspension was filtered, and the filtrate was extracted with ethyl acetate (3×500 mL). The combined organic extracts were washed with brine (150 mL), dried over anhydrous Na₂SO₄, filtered and concentrated under reduced pressure to afford 15.2 g of 8,9-difluorothiopyrano[3,4-c]isochromene-1,6(2H,4H)-dione and 4,5-difluoro-2-(5-hydroxy-3-oxo-3,6-dihydro-2H-thiopyran-4-yl)benzoic acid which was taken as such for next step.

Step ii: To a mixture of 5 g (1.86 mmol, 1.0 eq) of above prepared crude mixture of 8,9-difluorothiopyrano[3,4-c]isochromene-1,6(2H,4H)-dione and 4,5-difluoro-2-(5-hydroxy-3-oxo-3,6-dihydro-2H-thiopyran-4-yl)benzoic acid in a steel bomb at −35° C. was added 100 mL of 7 M methanolic ammonia. The vessel was sealed and the mixture was heated at 120° C. for 1 h. The mixture was then allowed to cool to room temperature and concentrated under reduced pressure. The residue was stirred with 10 vol of DMSO:Water (1:9) for 30 min to obtain a solid which was filtered and washed with water to afford 1.3 g (4.8 mmol, 26%) of 8,9-difluoro-2H-thiopyrano[3,4-c]isoquinoline-1,6(4H,5H)-dione (IVah). LCMS: m/z found 266.2 [M−H]—.

### 8,9-Difluoro-1-(methylamino)-1,5-dihydro-2H-thiopyrano[3,4-c]isoquinolin-6(4H)-one (Vba)

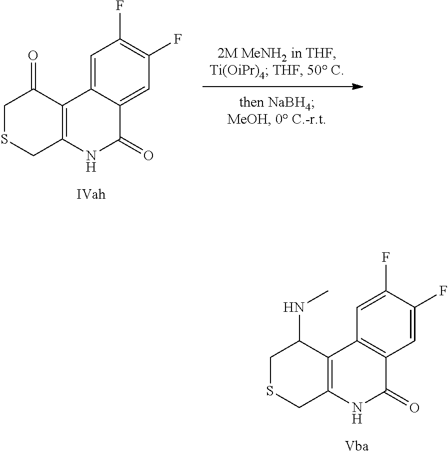 [SMILES: O=C1CSCc2[nH]c(=O)c3cc(F)c(F)cc3c21 ; CNC1CSCc2[nH]c(=O)c3cc(F)c(F)cc3c21]

Racemic 8,9-difluoro-1-(methylamino)-1,5-dihydro-2H-thiopyrano[3,4-c]isoquinolin-6(4H)-one was synthesized in an analogous manner as described above from 8,9-difluoro-2H-thiopyrano[3,4-c]isoquinoline-1,6(4H,5H)-dione (IVah), and methylamine. LCMS: m/z found 283.3 [M+H]⁺.

### 3-(3-Chloro-4-fluorophenyl)-1-(8,9-difluoro-6-oxo-1,4,5,6-tetrahydro-2H-thiopyrano[3,4-c]isoquinolin-1-yl)-1-methylurea (Compounds 187 and 188)

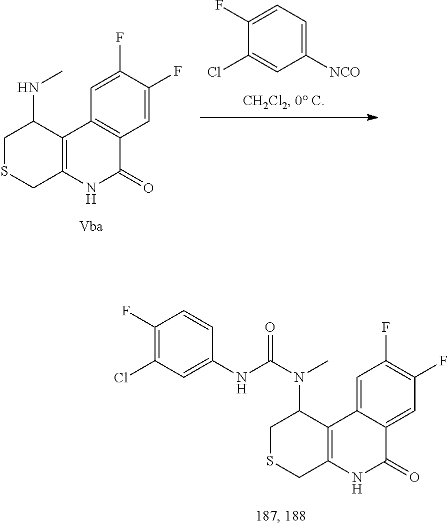

Racemic 3-(3-chloro-4-fluorophenyl)-1-(8,9-difluoro-6-oxo-1,4,5,6-tetrahydro-2H-thiopyrano[3,4-c]isoquinolin-1-yl)-1-methylurea was synthesized from 8,9-difluoro-1-(methylamino)-1,5-dihydro-2H-thiopyrano[3,4-c]isoquinolin-6(4H)-one (Vba) in an analogous manner as described above. The enantiomers were subsequently separated by preparative SFC: Method isocratic, Mobile phase MeOH:CO₂—40:60. Column: Chiralpak-IC (30×250 mm), 5μ, flow rate: 100 g/min.

Enantiomer I (Compound 187): LCMS: m/z found 454.1/456.1 [M+H]⁺, RT=5.42 min, (Method A); ¹H NMR (400 MHz, DMSO-d₆): δ 11.67 (br s, 1H), 8.56 (s, 1H), 8.11-8.06 (t, 1H) 7.83-7.80 (dd, 1H), 7.54-7.50 (m, 1H), 7.37-7.26 (m, 2H), 5.6 (s, 1H), 3.75 (d, 1H), 3.60 (d, 1H), 2.9 (d, 1H), 2.87 (d, 1H), 2.80 (s, 3H); Chiral analytical SFC: RT=1.80 min, Column: Chiralpak IC-3 (4.6×150 mm) 3 μm, 40% methanol, Flow rate: 3 g/min.

Enantiomer II (Compound 188): LCMS: m/z found 454.1/456.1 [M+H]⁺, RT=5.42 (Method A); ¹H NMR (400 MHz, DMSO-d₆): δ 11.67 (br s, 1H), 8.56 (s, 1H), 8.11-8.06 (t, 1H) 7.83-7.80 (dd, 1H), 7.54-7.50 (m, 1H), 7.37-7.26 (m, 2H), 5.6 (s, 1H), 3.75 (d, 1H), 3.60 (d, 1H), 2.9 (d, 1H), 2.87 (d, 1H), 2.80 (s, 3H); Chiral analytical SFC: RT=4.94 min, Column: Chiralpak IC-3 (4.6×150 mm) 3 μm, 40% methanol, Flow rate: 3 g/min.

### 3-(3-Cyano-4-fluorophenyl)-1-(8,9-difluoro-6-oxo-1,4,5,6-tetrahydro-2H-thiopyrano[3,4-c]isoquinolin-1-yl)-1-methylurea (Compounds 189 and 190)

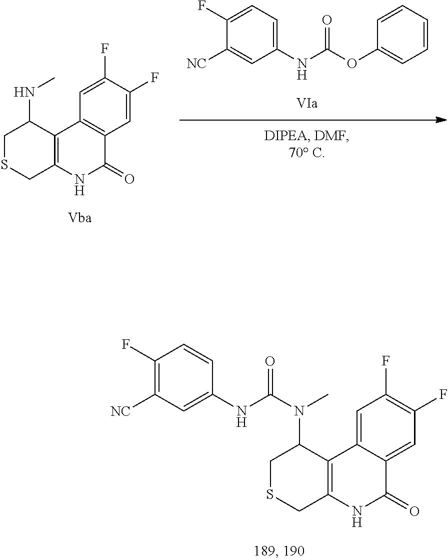 [SMILES: CNC1CSCc2[nH]c(=O)c3cc(F)c(F)cc3c21 ; N#Cc1cc(NC(=O)Oc2ccccc2)ccc1F ; CN(C(=O)Nc1ccc(F)c(C#N)c1)C1CSCc2[nH]c(=O)c3cc(F)c(F)cc3c21]

Racemic 3-(3-cyano-4-fluorophenyl)-1-(8,9-difluoro-6-oxo-1,4,5,6-tetrahydro-2H-thiopyrano[3,4-c]isoquinolin-1-yl)-1-methylurea was synthesized from 8,9-difluoro-1-(methylamino)-1,5-dihydro-2H-thiopyrano[3,4-c]isoquinolin-6(4H)-one (Vba) and phenyl (3-cyano-4-fluorophenyl)carbamate (VIa) in an analogous manner as described above. The enantiomers were subsequently separated by preparative SFC: Method isocratic, Mobile phase MeOH:CO₂—35:65. Column: Chiralpak-IC (30×250 mm), 5μ, flow rate: 70 g/min.

Enantiomer I (Compound 189): LCMS: m/z found 445.2 [M+H]⁺, RT=5.21 min, (Method A); ¹H NMR (400 MHz, DMSO-d₆): δ 11.67 (br s, 1H), 8.72 (s, 1H), 8.12-8.04 (m, 2H) 7.92-7.85 (m, 1H), 7.54-7.44 (t, 1H), 7.35-7.22 (dd, 1H), 5.64 (s, 1H), 3.8-3.7 (d, 1H), 3.6-3.5 (d, 1H), 3.18-3.15 (dd, 1H), 3.0-2.9 (dd, 1H), 2.81 (s, 3H); Chiral analytical SFC: RT=2.21 min, Column: Chiralpak IC-3 (4.6×150 mm) 3 μm, 35% methanol, Flow rate: 3 g/min.

Enantiomer II (Compound 190): LCMS: m/z found 445.2 [M+H]⁺, RT=5.21 min, (Method A); ¹H NMR (400 MHz, DMSO-d₆): δ 11.67 (br s, 1H), 8.72 (s, 1H), 8.12-8.04 (m, 2H) 7.92-7.85 (m, 1H), 7.54-7.44 (t, 1H), 7.35-7.22 (dd, 1H), 5.64 (s, 1H), 3.8-3.7 (d, 1H), 3.6-3.5 (d, 1H), 3.18-3.15 (dd, 1H), 3.0-2.9 (dd, 1H), 2.81 (s, 3H); Chiral analytical SFC: RT=2.66 min, Column: Chiralpak IC-3 (4.6×150 mm) 3 μm, 35% methanol, Flow rate: 3 g/min.

### 8-Fluoro-2H-thiopyrano[3,4-c]isoquinoline-1,6(4H,5H)-dione (IVai)

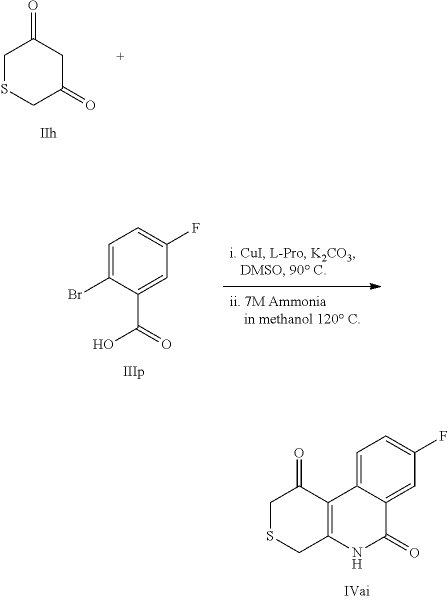 [SMILES: O=C1CSCC(=O)C1 ; O=C(O)c1cc(F)ccc1Br ; O=C1CSCc2[nH]c(=O)c3cc(F)ccc3c21]

8-Fluoro-2H-thiopyrano[3,4-c]isoquinoline-1,6(4H,5H)-dione was synthesized in an analogous manner as described above for IVah, from 2H-thiopyran-3,5(4H,6H)-dione (IIh) and 5-fluoro-2-bromo-benzoic acid (IIIp). ¹H NMR (400 MHz, DMSO-d₆): δ 12.45 (br s, 1H), 9.03-8.97 (m, 1H), 8.12 (dd, 1H), 4.71 (br s, 2H), 4.18 (br s, 2H), 1.42 (s, 9H). LCMS: m/z found 250.17 [M+H]⁺. Note: Reaction was repeated multiple times on 5 g scale with consistent results.

### 8-Fluoro-1-(methylamino)-1,5-dihydro-2H-thiopyrano[3,4-c]isoquinolin-6(4H)-one (Vbb)

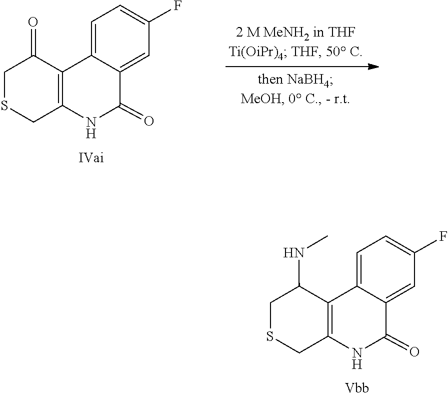 [SMILES: O=C1CSCc2[nH]c(=O)c3cc(F)ccc3c21 ; CNC1CSCc2[nH]c(=O)c3cc(F)ccc3c21]

Racemic 8-fluoro-1-(methylamino)-1,5-dihydro-2H-thiopyrano[3,4-c]isoquinolin-6(4H)-one was synthesized in an analogous manner as described above, from 8-fluoro-2H-thiopyrano[3,4-c]isoquinoline-1,6(4H,5H)-dione (IVai), and methylamine. LCMS: m/z found 263.29 [M−H]⁻. Note: Reaction was repeated multiple times on 0.5 g scale with consistent results.

### 3-(3-Chloro-4-fluorophenyl)-1-(8-fluoro-6-oxo-1,4,5,6-tetrahydro-2H-thiopyrano[3,4-c]isoquinolin-1-yl)-1-methylurea (Compounds 183 and 184)

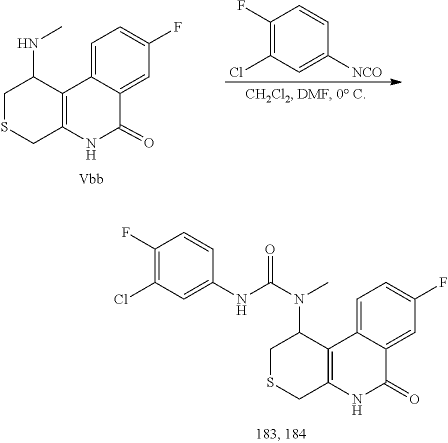

Racemic 3-(3-chloro-4-fluorophenyl)-1-(8-fluoro-6-oxo-1,4,5,6-tetrahydro-2H-thiopyrano[3,4-c]isoquinolin-1-yl)-1-methylurea was synthesized from 8-fluoro-1-(methylamino)-1,5-dihydro-2H-thiopyrano[3,4-c]isoquinolin-6(4H)-one (Vbb) in an analogous manner as described above, except for conducting the reaction in a 1:1 v/v mixture of dichloromethane and DMF as a solvent. The enantiomers were subsequently separated by preparative SFC: Method isocratic, Mobile phase MeOH:CO₂—40:60. Column: Chiralpak-IC (30×250 mm), 5μ, flow rate: 100 g/min.

Enantiomer I (Compound 183): LCMS: m/z found 436.1/438.1 [M+H]⁺, RT=5.15 min, (Method A); ¹H NMR (400 MHz, DMSO-d₆): δ 11.67 (br s, 1H), 8.53 (s, 1H), 7.91-7.86 (m, 2H) 7.71-7.68 (m, 1H), 7.54-7.45 (m, 2H), 7.34-7.30 (t, 1H), 5.68 (s, 1H), 3.82 (d, 1H), 3.56 (d, 1H), 3.12 (d, 1H), 2.96 (d, 1H), 2.79 (s, 3H); Chiral analytical SFC: RT=1.90 min, Column: Chiralpak IC-3 (4.6×150 mm) 3 μm, 40% methanol, Flow rate: 3 g/min.

Enantiomer II (Compound 184): LCMS: m/z found 436.1/438.1 [M+H]⁺, RT=5.15 min, (Method A); ¹H NMR (400 MHz, DMSO-d₆): δ 11.67 (br s, 1H), 8.53 (s, 1H), 7.91-7.86 (m, 2H) 7.71-7.68 (m, 1H), 7.54-7.45 (m, 2H), 7.34-7.30 (t, 1H), 5.68 (s, 1H), 3.82 (d, 1H), 3.56 (d, 1H), 3.12 (d, 1H), 2.96 (d, 1H), 2.79 (s, 3H); Chiral analytical SFC: RT=2.56 min, Column: Chiralpak IC-3 (4.6×150 mm) 3 μm, 40% methanol, Flow rate: 3 g/min.

### 3-(3-Cyano-4-fluorophenyl)-1-(8-fluoro-6-oxo-1,4,5,6-tetrahydro-2H-thiopyrano[3,4-c]isoquinolin-1-yl)-1-methylurea (Compounds 185 and 186)

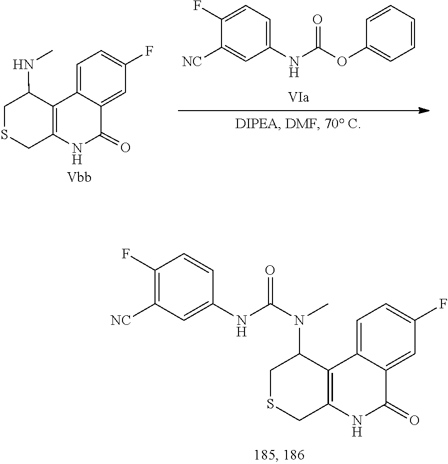 [SMILES: CNC1CSCc2[nH]c(=O)c3cc(F)ccc3c21 ; N#Cc1cc(NC(=O)Oc2ccccc2)ccc1F ; CN(C(=O)Nc1ccc(F)c(C#N)c1)C1CSCc2[nH]c(=O)c3cc(F)ccc3c21]

Racemic 3-(3-cyano-4-fluorophenyl)-1-(8-fluoro-6-oxo-1,4,5,6-tetrahydro-2H-thiopyrano[3,4-c]isoquinolin-1-yl)-1-methylurea was synthesized from 8-fluoro-1-(methylamino)-1,5-dihydro-2H-thiopyrano[3,4-c]isoquinolin-6(4H)-one (Vbb) and phenyl (3-cyano-4-fluorophenyl)carbamate (VIa) in an analogous manner as described above. The enantiomers were subsequently separated by preparative SFC: Method isocratic, Mobile phase MeOH:CO₂—30:70. Column: Chiralpak-OX-H (30×250 mm), 5μ, flow rate: 70 g/min.

Enantiomer I (Compound 185): LCMS: m/z found 427.2 [M+H]⁺, RT=4.73 min, (Method A); ¹H NMR (400 MHz, DMSO-d₆): δ 11.58 (br s, 1H), 8.71 (s, 1H), 8.11-8.09 (m, 1H) 7.91-7.86 (m, 2H), 7.71-7.66 (m, 1H), 7.49-7.44 (m, 2H), 5.68 (s, 1H), 3.78 (d, 1H), 3.56 (d, 1H), 3.17 (d, 1H), 3.13 (d, 1H), 2.81 (s, 3H); Chiral analytical SFC: RT=2.10 min, Column: Chiralpak OX-3 (4.6×150 mm) 3 μm, 35% methanol, Flow rate: 3 g/min.

Enantiomer II (Compound 186): LCMS: m/z found 427.2 [M+H]⁺, RT=4.73 min, (Method A); ¹H NMR (400 MHz, DMSO-d₆): δ 11.58 (br s, 1H), 8.71 (s, 1H), 8.11-8.09 (m, 1H) 7.91-7.86 (m, 2H), 7.71-7.66 (m, 1H), 7.49-7.44 (m, 2H), 5.68 (s, 1H), 3.78 (d, 1H), 3.56 (d, 1H), 3.17 (d, 1H), 3.13 (d, 1H), 2.81 (s, 3H); Chiral analytical SFC: RT=2.60 min, Column: Chiralpak OX-3 (4.6×150 mm) 3 μm, 35% methanol, Flow rate: 3 g/min.

### 8-Fluoro-1-(methylamino)-1,5-dihydro-2H-thiopyrano[3,4-c]isoquinolin-6(4H)-one 3-oxide (Vbc)

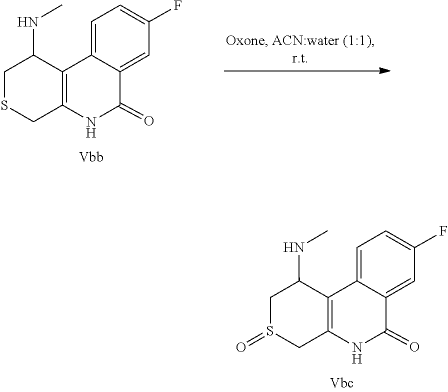 [SMILES: CNC1CSCc2[nH]c(=O)c3cc(F)ccc3c21 ; CNC1CS(=O)Cc2[nH]c(=O)c3cc(F)ccc3c21]

To a stirred solution of 500 mg (1.89 mmol, 1.0 eq) of 8-fluoro-1-(methylamino)-1,5-dihydro-2H-thiopyrano[3,4-c]isoquinolin-6(4H)-one (Vbb) in 5 mL of acetonitrile:water (1:1, v/v) at room temperature, 523 mg (1.7 mmol, 0.9 eq) of oxone was added and the resulting reaction mixture was stirred for 4 h. The mixture was then concentrated and diluted with methanol (10 mL). After filtering the suspension, the filtrate was concentrated under reduced pressure to afford crude 8-fluoro-1-(methylamino)-1,5-dihydro-2H-thiopyrano[3,4-c]isoquinolin-6(4H)-one 3-oxide (Vbc, 800 mg), which was used without further purification in the next steps. LCMS: m/z found 281.18 [M−H]⁻.

### 3-(3-Chloro-4-fluorophenyl)-1-(8-fluoro-3-oxido-6-oxo-1,4,5,6-tetrahydro-2H-thiopyrano[3,4-c]isoquinolin-1-yl)-1-methylurea (Compounds 193, 194, 195, and 196)

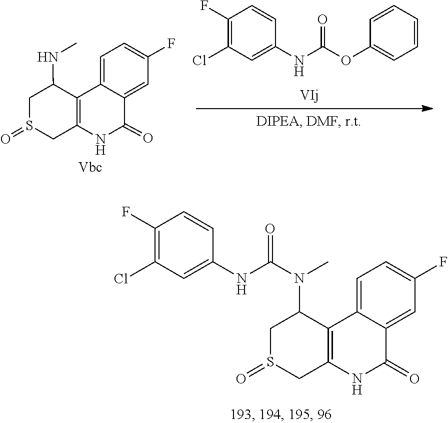

To a stirred solution of 400 mg (1.43 mmol, 1 eq.) of 8-fluoro-1-(methylamino)-1,5-dihydro-2H-thiopyrano[3,4-c]isoquinolin-6(4H)-one 3-oxide (Vbc) in 3 mL of DMF at room temperature, 0.76 mL (4.28 mmol, 3 eq.) of DIPEA, 378 mg (1.43 mmol, 1 eq.) of phenyl (3-chloro-4-fluorophenyl)carbamate (VIj) were added and the resulting reaction mixture was stirred for 16 hours. The reaction mixture was then diluted with cold water (15 mL) and stirred for 30 minutes. The resulting suspension was filtered and the solid was washed with 5 mL of water. The crude solid (150 mg) was triturated with ethyl acetate (5 mL) to afford 110 mg (0.24 mmol, 26% yield over two steps) of 3-(3-cyano-4-fluorophenyl)-1-(8,9-difluoro-6-oxo-1,4,5,6-tetrahydro-2H-thiopyrano[3,4-c]isoquinolin-1-yl)-1-methylurea. 110 mg of this product was subjected to chiral preparative SFC: Method isocratic, Mobile phase MeOH:CO₂—35:75. Column: DCPAK-P4VP (21×250) mm, 5μ, flow rate: 65 g/min, to afford 75 mg of one racemic diastereoisomer and 65 mg of another racemic diastereoisomer. Each of these two racemates were subjected to chiral preparative SFC: Column: Chiralcel OX-H (21×250) mm, 5μ, Method isocratic, Mobile phase MeOH:CO₂—40:60, flow rate: 60 g/min, and, respectively, Mobile phase MeOH:CO₂—45:55, flow rate: 110 g/min, to afford 20 mg of compound 193 and 22 mg of compound 194, and, respectively, 13 mg of compound 195, and 12.8 mg of compound 196.

Stereoisomer I (Compound 193): LCMS: m/z found 452.2/454.2 [M+H]⁺, RT=5.16 min, (Method A); ¹H NMR (400 MHz, DMSO-d₆): δ 11.60 (br s, 1H), 8.58 (brs, 1H), 7.91-7.88 (m, 2H) 7.74-7.69 (m, 1H), 7.54-7.49 (m, 2H), 7.33 (t, 1H), 6.02 (t, 1H), 4.12 (s, 2H), 3.57-3.52 (m, 1H), 3.27-3.24 (m, 1H), 2.61 (s, 3H); Chiral analytical SFC: RT=2.63 min, Column: Chiralcel OX-3 (4.6×150 mm) 3 μm, 50% methanol, Flow rate: 3 g/min.

Stereoisomer II (Compound 194, enantiomer of 193): LCMS: m/z found 452.2/454.2 [M+H]⁺, RT=5.16 min, (Method A); ¹H NMR (400 MHz, DMSO-d₆): δ 11.60 (br s, 1H), 8.58 (brs, 1H), 7.91-7.88 (m, 2H) 7.74-7.69 (m, 1H), 7.54-7.49 (m, 2H), 7.33 (t, 1H), 6.02 (t, 1H), 4.12 (s, 2H), 3.57-3.52 (m, 1H), 3.27-3.24 (m, 1H), 2.61 (s, 3H); Chiral analytical SFC: RT=4.18 min, Column: Chiralcel OX-3 (4.6×150 mm) 3 μm, 50% methanol, Flow rate: 3 g/min.

Stereoisomer III (Compound 195): LCMS: m/z found 452.2/454.2 [M+H]⁺, RT=5.10 min, (Method A); ¹H NMR (400 MHz, DMSO-d₆): δ 11.60 (br s, 1H), 8.62 (brs, 1H), 7.89-7.86 (m, 2H) 7.69-7.64 (m, 1H), 7.54-7.47 (m, 2H), 7.33 (t, 1H), 6.08 (t, 1H), 4.30 (d, 1H), 3.84 (d, 1H), 3.50-3.45 (m, 1H), 3.19-3.14 (m, 1H), 2.57 (s, 3H); Chiral analytical SFC: RT=2.81 min, Column: Chiralcel OX-3 (4.6×150 mm) 3 μm, 50% methanol, Flow rate: 3 g/min.

Stereoisomer IV (Compound 196, enantiomer of 196): LCMS: m/z found 452.2/454.2 [M+H]⁺, RT=5.10 min, (Method A); ¹H NMR (400 MHz, DMSO-d₆): δ 11.60 (br s, 1H), 8.62 (brs, 1H), 7.89-7.86 (m, 2H) 7.69-7.64 (m, 1H), 7.54-7.47 (m, 2H), 7.33 (t, 1H), 6.08 (t, 1H), 4.30 (d, 1H), 3.84 (d, 1H), 3.50-3.45 (m, 1H), 3.19-3.14 (m, 1H), 2.57 (s, 3H); Chiral analytical SFC: RT=4.59 min, Column: Chiralcel OX-3 (4.6×150 mm) 3 μm, 50% methanol, Flow rate: 3 g/min.

### 3-(3-Cyano-4-fluorophenyl)-1-(8-fluoro-3-oxido-6-oxo-1,4,5,6-tetrahydro-2H1-thiopyrano[3,4-c]isoquinolin-1-yl)-1-methylurea (Compounds 199, 200, 201, and 202)

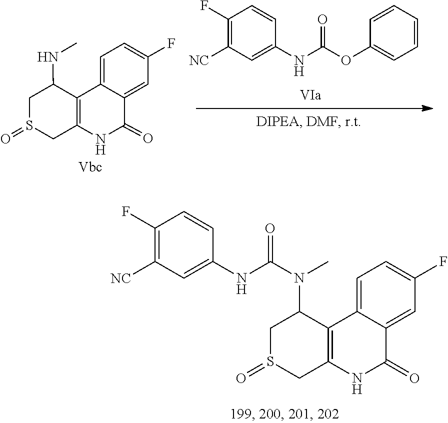

3-(3-Cyano-4-fluorophenyl)-1-(8-fluoro-3-oxido-6-oxo-1,4,5,6-tetrahydro-2H-thiopyrano[3,4-c]isoquinolin-1-yl)-1-methylurea was synthesized as a mixture of stereoisomers from 8-fluoro-1-(methylamino)-1,5-dihydro-2H-thiopyrano[3,4-c]isoquinolin-6(4H)-one 3-oxide (Vbc) and phenyl (3-cyano-4-fluorophenyl)carbamate (VIa) in an analogous manner as described above. The stereoisomers were subsequently separated by preparative SFC: Method isocratic, Mobile phase MeOH:CO₂—40:60, Column: DCPAK-P4VP (21×250) mm, 5μ, flow rate: 60 g/min, to isolate each of the compounds 201 and 202, followed by a second preparative SFC: Method isocratic, Mobile phase MeOH:CO₂—40:60, Column: Chiralcel OD-H (30×250) mm, 5μ, flow rate: 100 g/min, on the remaining mixture, to isolate each of the compounds 199 and 200.

Stereoisomer I (Compound 199): LCMS: m/z found 443.2 [M+H]⁺, RT=5.33 min, (Method A); ¹H NMR (400 MHz, DMSO-d₆): δ 11.60 (br s, 1H), 8.82 (brs, 1H), 8.10-8.08 (m, 1H) 7.91-7.87 (m, 2H), 7.73-7.68 (m, 1H), 7.54-7.45 (m, 2H), 6.09 (t, 1H), 4.32 (d, 1H), 3.86 (d, 1H), 3.58-3.48 (m, 1H), 3.20-3.15 (m, 1H), 2.59 (s, 3H); Chiral analytical SFC: RT=7.68 min, Column: Chiralcel OX-3 (4.6×150 mm) 3 μm, 35% methanol, Flow rate: 3 g/min.

Stereoisomer II (Compound 200, enantiomer of 199): LCMS: m/z found 443.2 [M+H]⁺, RT=5.33 min, (Method A); ¹H NMR (400 MHz, DMSO-d₆): δ 11.60 (br s, 1H), 8.83 (brs, 1H), 8.10-8.08 (m, 1H) 7.92-7.87 (m, 2H), 7.74-7.67 (m, 1H), 7.54-7.45 (m, 2H), 6.09 (t, 1H), 4.32 (d, 1H), 3.86 (d, 1H), 3.53-3.51 (m, 1H), 3.20-3.15 (m, 1H), 2.59 (s, 3H); Chiral analytical SFC: RT=13.59 min, Column: Chiralcel OX-3 (4.6×150 mm) 3 μm, 35% methanol, Flow rate: 3 g/min.

Stereoisomer III (Compound 201): LCMS: m/z found 443.1 [M+H]⁺, RT=5.37 min, (Method A); ¹H NMR (400 MHz, DMSO-d₆): δ 11.60 (br s, 1H), 8.75 (brs, 1H), 8.10-8.08 (m, 1H) 7.89-7.87 (m, 2H), 7.72-7.66 (m, 1H), 7.51-7.44 (m, 2H), 6.02 (t, 1H), 4.10 (s, 2H), 3.58-3.53 (m, 1H), 3.31-3.25 (m, 1H), 2.61 (s, 3H); Chiral analytical SFC: RT=7.09 min, Column: Chiralcel OX-3 (4.6×150 mm) 3 μm, 35% methanol, Flow rate: 3 g/min.

Stereoisomer IV (Compound 202, enantiomer of 201): LCMS: m/z found 443.1 [M+H]⁺, RT=5.37 min, (Method A); ¹H NMR (400 MHz, DMSO-d₆): δ 11.60 (br s, 1H), 8.76 (brs, 1H), 8.10-8.08 (m, 1H) 7.91-7.86 (m, 2H), 7.75-7.70 (m, 1H), 7.52-7.44 (m, 2H), 6.03 (t, 1H), 4.12 (s, 2H), 3.57-3.52 (m, 1H), 3.31-3.25 (m, 1H), 2.62 (s, 3H); Chiral analytical SFC: RT=10.05 min, Column: Chiralcel OX-3 (4.6×150 mm) 3 μm, 35% methanol, Flow rate: 3 g/min.

### 8,9-Difluoro-1-(methylamino)-1,5-dihydro-2H-thiopyrano[3,4-c]isoquinolin-6(4H1)-one 3-oxide (Vbf)

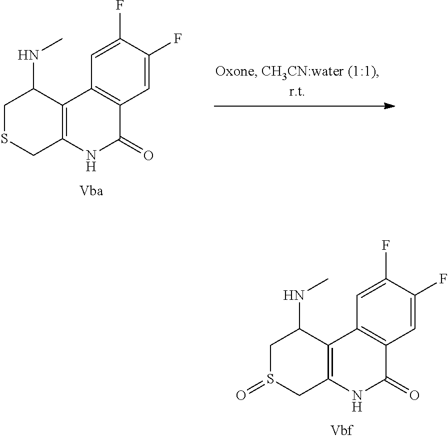 [SMILES: CNC1CSCc2[nH]c(=O)c3cc(F)c(F)cc3c21 ; CNC1CS(=O)Cc2[nH]c(=O)c3cc(F)c(F)cc3c21]

To a stirred solution of 850 mg (282 mmol, 1 eq) of 8,9-difluoro-1-(methylamino)-1,5-dihydro-2H-thiopyrano[3,4-c]isoquinolin-6(4H)-one (Vba) in 10 mL of acetonitrile:water (1:1) at room temperature, 740 mg (2.44 mmol, 0.8 eq) of oxone was added and the resulting reaction mixture was stirred for 6 h. The mixture was then concentrated and diluted with methanol (20 mL). After filtering the suspension, the filtrate was concentrated under reduced pressure to afford crude (700 mg) of 8,9-difluoro-1-(methylamino)-1,5-dihydro-2H-thiopyrano[3,4-c]isoquinolin-6(4H)-one 3-oxide (Vbf). This material was used without further purification in the next steps. LCMS: m/z found 299.24 [M+H]⁺.

### 3-(3-Chloro-4-fluorophenyl)-1-(8,9-difluoro-3-oxido-6-oxo-1,4,5,6-tetrahydro-2H-thiopyrano[3,4-c]isoquinolin-1-yl)-1-methylurea (Compounds, 207, 208, 209, and 210)

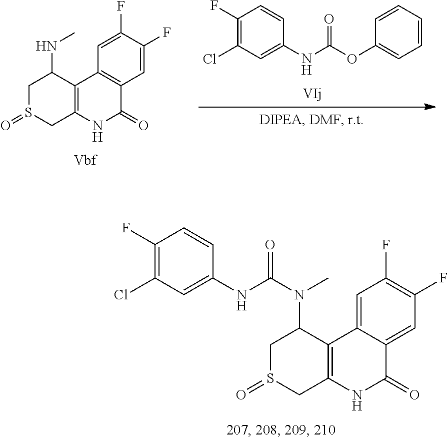 [SMILES: CNC1CS(=O)Cc2[nH]c(=O)c3cc(F)c(F)cc3c21 ; O=C(Nc1ccc(F)c(Cl)c1)Oc1ccccc1 ; CN(C(=O)Nc1ccc(F)c(Cl)c1)C1CS(=O)Cc2[nH]c(=O)c3cc(F)c(F)cc3c21]

3-(3-Chloro-4-fluorophenyl)-1-(8,9-difluoro-3-oxido-6-oxo-1,4,5,6-tetrahydro-2H-thiopyrano[3,4-c]isoquinolin-1-yl)-1-methylurea was synthesized as a mixture of stereoisomers from 8,9-difluoro-1-(methylamino)-1,5-dihydro-2H-thiopyrano[3,4-c]isoquinolin-6(4H)-one 3-oxide (Vbf) and phenyl (3-chloro-4-fluorophenyl)carbamate (VIj) in an analogous manner as described above. The crude material was separated into diastereoisomeric racemates by reverse phase preparative HPLC. Method 10 min gradient (Ammonium Bicarbonate in water)/Acetonitrile, Column: X-bridge C18 (30×150) mm, 5μ, flow rate 18 mL/min. Each of these racemates were further separated into respective enantiomers by preparative SFC: Method isocratic, Mobile phase MeOH:CO₂—40:60, Column: Chiralcel OX-H (21×250) mm, 5μ, flow rate: 70 g/min.

Stereoisomer I (Compound 207): LCMS: m/z found 470.2/472.2 [M+H]⁺, RT=6.23 min, (Method A); ¹H NMR (400 MHz, DMSO-d₆): δ 11.31 (br s, 1H), 8.67 (brs, 1H), 8.16-8.11 (m, 1H), 7.84-7.82 (m, 1H), 7.53-7.49 (m, 1H), 7.43-7.33 (m, 2H), 6.03 (t, 1H), 4.31 (d, 1H), 3.86 (d, 1H), 3.52-3.51 (m, 1H), 3.16-3.11 (m, 1H), 2.61 (s, 3H); Chiral analytical SFC: RT=1.21 min, Column: Chiralpak AS-3 (4.6×150 mm) 3 μm, 50% methanol, Flow rate: 3 g/min.

Stereoisomer II (Compound 208, enantiomer of 207): LCMS: m/z found 470.2/472.2 [M+H]⁺, RT=6.23 min, (Method A); ¹H NMR (400 MHz, DMSO-d₆): δ 11.31 (br s, 1H), 8.67 (brs, 1H), 8.16-8.11 (m, 1H), 7.84-7.82 (m, 1H), 7.53-7.49 (m, 1H), 7.43-7.33 (m, 2H), 6.03 (t, 1H), 4.31 (d, 1H), 3.86 (d, 1H), 3.52-3.51 (m, 1H), 3.16-3.11 (m, 1H), 2.61 (s, 3H); Chiral analytical SFC: RT=1.62 min, Column: Chiralpak AS-3 (4.6×150 mm) 3 m, 50% methanol, Flow rate: 3 g/min.

Stereoisomer III (Compound 209): LCMS: m/z found 470.1/472.1 [M+H]⁺, RT=6.33 min, (Method A); ¹H NMR (400 MHz, DMSO-d₆): δ 11.11 (br s, 1H), 8.62 (brs, 1H), 8.15-8.10 (m, 1H), 7.84-7.82 (m, 1H), 7.53-7.49 (m, 1H), 7.43-7.32 (m, 2H), 6.01 (t, 1H), 4.18-4.10 (m, 2H), 3.59-3.56 (m, 1H), 3.32-3.25 (m, 1H), 2.64 (s, 3H); Chiral analytical SFC: RT=1.03 min, Column: Chiralpak AS-3 (4.6×150 mm) 3 μm, 50% methanol, Flow rate: 3 g/min.

Stereoisomer IV (Compound 210, enantiomer of 209): LCMS: m/z found 470.1/472.1 [M+H]⁺, RT=6.33 min, (Method A); ¹H NMR (400 MHz, DMSO-d₆): δ 11.11 (br s, 1H), 8.62 (brs, 1H), 8.15-8.10 (m, 1H), 7.84-7.82 (m, 1H), 7.53-7.49 (m, 1H), 7.43-7.32 (m, 2H), 6.01 (t, 1H), 4.18-4.10 (m, 2H), 3.59-3.56 (m, 1H), 3.32-3.25 (m, 1H), 2.64 (s, 3H); Chiral analytical SFC: RT=1.35 min, Column: Chiralpak AS-3 (4.6×150 mm) 3 μm, 50% methanol, Flow rate: 3 g/min.

### 3-(3-Cyano-4-fluorophenyl)-1-(8,9-difluoro-3-oxido-6-oxo-1,4,5,6-tetrahydro-2H1-thiopyrano[3,4-c]isoquinolin-1-yl)-1-methylurea (Compounds 211, 212, 213, and 214)

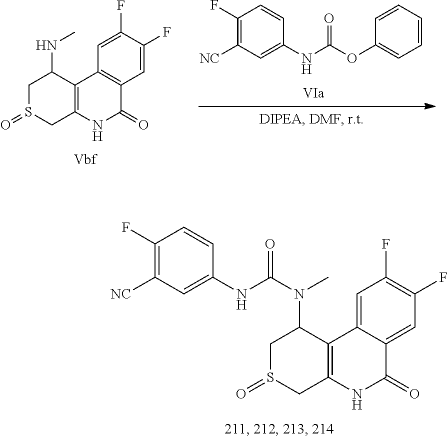 [SMILES: CNC1CS(=O)Cc2[nH]c(=O)c3cc(F)c(F)cc3c21 ; N#Cc1cc(NC(=O)Oc2ccccc2)ccc1F ; CN(C(=O)Nc1ccc(F)c(C#N)c1)C1CS(=O)Cc2[nH]c(=O)c3cc(F)c(F)cc3c21]

3-(3-Cyano-4-fluorophenyl)-1-(8,9-difluoro-3-oxido-6-oxo-1,4,5,6-tetrahydro-2H-thiopyrano[3,4-c]isoquinolin-1-yl)-1-methylurea was synthesized as a mixture of stereoisomers from 8,9-difluoro-1-(methylamino)-1,5-dihydro-2H-thiopyrano[3,4-c]isoquinolin-6(4H)-one 3-oxide (Vbf) and phenyl (3-cyano-4-fluorophenyl)carbamate (VIa) in an analogous manner as described above. The stereoisomers were subsequently separated by preparative SFC: Method isocratic, Mobile phase MeOH:CO₂—40:60, Column: Chiralcel OX (30×250) mm, 5μ, flow rate: 70 g/min, to isolate each of the compounds 213 and 214, followed by a second preparative SFC: Method isocratic, Mobile phase MeOH:CO₂—40:60, Column: DCPAK-P4VP (21×250) mm, 5μ, flow rate: 60 g/min, on the remaining mixture, to isolate each of the compounds 211 and 212.

Stereoisomer I (Compound 211): LCMS: m/z found 461.2 [M+H]⁺, RT=6.00 min, (Method A); ¹H NMR (400 MHz, DMSO-d₆): δ 11.69 (s, 1H), 8.83 (brs, 1H), 8.16-8.11 (m, 1H) 8.04-8.02 (m, 1H), 7.91-7.87 (m, 1H), 7.49 (t, 1H), 7.41-7.36 (m, 1H), 6.03 (t, 1H), 4.33 (d, 1H), 3.87 (d, 1H), 3.53-3.48 (m, 1H), 3.18-3.13 (m, 1H), 2.62 (s, 3H); Chiral analytical SFC: RT=4.12 min, Column: Chiralcel OX-3 (4.6×150 mm) 3 μm, 40% methanol, Flow rate: 3 g/min.

Stereoisomer II (Compound 212, enantiomer of 211): LCMS: m/z found 461.2 [M+H]⁺, RT=6.00 min, (Method A); ¹H NMR (400 MHz, DMSO-d₆): δ 11.69 (s, 1H), 8.83 (brs, 1H), 8.16-8.11 (m, 1H) 8.04-8.02 (m, 1H), 7.91-7.87 (m, 1H), 7.49 (t, 1H), 7.41-7.36 (m, 1H), 6.03 (t, 1H), 4.33 (d, 1H), 3.87 (d, 1H), 3.53-3.48 (m, 1H), 3.18-3.13 (m, 1H), 2.62 (s, 3H); Chiral analytical SFC: RT=6.84 min, Column: Chiralcel OX-3 (4.6×150 mm) 3 m, 40% methanol, Flow rate: 3 g/min.

Stereoisomer III (Compound 213): LCMS: m/z found 461.3 [M+H]⁺, RT=6.00 min (Method A); ¹H NMR (400 MHz, DMSO-d₆): δ 11.71 (s, 1H), 8.78 (brs, 1H), 8.15-8.10 (m, 1H) 8.05-8.03 (m, 1H), 7.90-7.86 (m, 1H), 7.48 (t, 1H), 7.38-7.33 (m, 1H), 6.01 (t, 1H), 4.14 (s, 2H), 3.58-3.54 (m, 1H), 3.33-3.25 (m, 1H), 2.65 (s, 3H); Chiral analytical SFC: RT=4.65 min, Column: Chiralcel OX-3 (4.6×150 mm) 3 μm, 40% methanol, Flow rate: 3 g/min.

Stereoisomer IV (Compound 214, enantiomer of 213): LCMS: m/z found 461.3 [M+H]⁺, RT=6.00 min, (Method A); ¹H NMR (400 MHz, DMSO-d₆): δ 11.71 (s, 1H), 8.78 (brs, 1H), 8.15-8.10 (m, 1H) 8.05-8.03 (m, 1H), 7.90-7.86 (m, 1H), 7.48 (t, 1H), 7.38-7.33 (m, 1H), 6.01 (t, 1H), 4.14 (s, 2H), 3.58-3.54 (m, 1H), 3.33-3.25 (m, 1H), 2.65 (s, 3H); Chiral analytical SFC: RT=10.05 min, Column: Chiralcel OX-3 (4.6×150 mm) 3 μm, 40% methanol, Flow rate: 3 g/min.

### 8-Fluoro-1-(methylamino)-1,5-dihydro-2H1-thiopyrano[3,4-c]isoquinolin-6(4H1)-one 3,3-dioxide (Vbd)

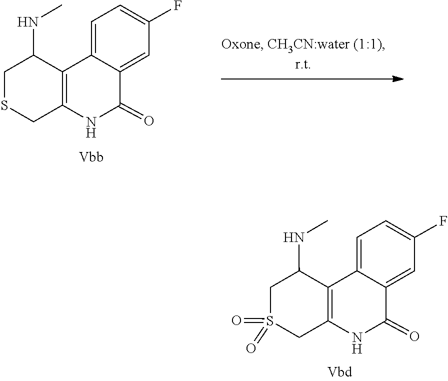 [SMILES: CNC1CSCc2[nH]c(=O)c3cc(F)ccc3c21 ; CNC1CS(=O)(=O)Cc2[nH]c(=O)c3cc(F)ccc3c21]

To a stirred solution of 600 mg (2.26 mmol, 1 eq) of 8-fluoro-1-(methylamino)-1,5-dihydro-2H-thiopyrano[3,4-c]isoquinolin-6(4H)-one (Vbb) in 12 mL of acetonitrile: water (1:1, v/v) at room temperature, 2.1 g (6.8 mmol, 3 eq) of oxone was added and the resulting reaction mixture was stirred for 16 h. The mixture was then concentrated, and the residue was diluted with methanol (15 mL). After filtering the suspension, the filtrate was concentrated under reduced pressure to afford 800 mg of crude product. This product was triturated with 20% methanol in DCM (10 mL) to obtain 8-fluoro-1-(methylamino)-1,5-dihydro-2H-thiopyrano[3,4-c]isoquinolin-6(4H)-one 3,3-dioxide (Vbd) which was used without further purification in the next steps. LCMS: m/z found 297.24 [M+H]⁺.

### 3-(3-Chloro-4-fluorophenyl)-1-(8-fluoro-3,3-dioxido-6-oxo-1,4,5,6-tetrahydro-2H-thiopyrano[3,4-c]isoquinolin-1-yl)-1-methylurea (Compounds 197 and 198)

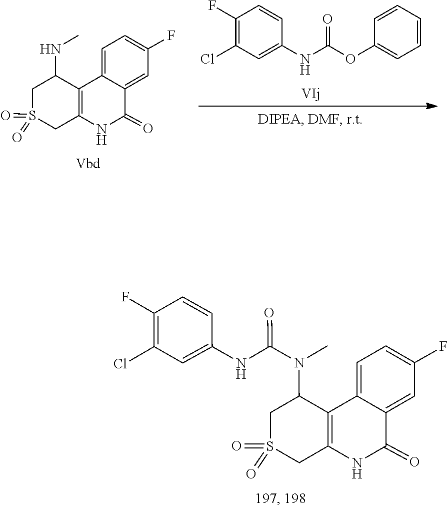 [SMILES: CNC1CS(=O)(=O)Cc2[nH]c(=O)c3cc(F)ccc3c21 ; O=C(Nc1ccc(F)c(Cl)c1)Oc1ccccc1 ; CN(C(=O)Nc1ccc(F)c(Cl)c1)C1CS(=O)(=O)Cc2[nH]c(=O)c3cc(F)ccc3c21]

Racemic 3-(3-chloro-4-fluorophenyl)-1-(8-fluoro-3,3-dioxido-6-oxo-1,4,5,6-tetrahydro-2H-thiopyrano[3,4-c]isoquinolin-1-yl)-1-methylurea was synthesized from 8-fluoro-1-(methylamino)-1,5-dihydro-2H-thiopyrano[3,4-c]isoquinolin-6(4H)-one 3,3-dioxide (Vbd) and phenyl (3-chloro-4-fluorophenyl)carbamate (VIj) in an analogous manner as described above. The enantiomers were subsequently separated by preparative SFC: Method isocratic, Mobile phase MeOH:CO₂—40:60. Column: Chiralcel OD (30×250 mm), 5μ, flow rate: 120 g/min.

Enantiomer I (Compound 197): LCMS: m/z found 468.2/470.2 [M+H]⁺, RT=5.41 min, (Method A); ¹H NMR (400 MHz, DMSO-d₆): δ 11.60 (br s, 1H), 8.64 (brs, 1H), 7.93-7.88 (m, 2H) 7.77-7.72 (m, 1H), 7.54-7.50 (m, 2H), 7.34 (t, 1H), 6.09 (t, 1H), 4.73 (d, 1H), 4.21-4.16 (m, 1H), 3.84-3.79 (m, 1H), 3.62-3.57 (m, 1H), 2.62 (s, 3H); Chiral analytical SFC: RT=2.01 min, Column: Chiralcel OD-3 (4.6×150 mm) 3 μm, 35% methanol, Flow rate: 3 g/min.

Enantiomer II (Compound 198): LCMS: m/z found 468.2/470.2 [M+H]⁺, RT=5.41 (Method A); ¹H NMR (400 MHz, DMSO-d₆): δ 11.60 (br s, 1H), 8.63 (brs, 1H), 7.92-7.89 (m, 2H) 7.73-7.69 (m, 1H), 7.53-7.49 (m, 2H), 7.34 (t, 1H), 6.08 (t, 1H), 4.70 (d, 1H), 4.20-4.15 (m, 1H), 3.82-3.78 (m, 1H), 3.61-3.55 (m, 1H), 2.61 (s, 3H); Chiral analytical SFC: RT=3.37 min, Column: Chiralcel OD-3 (4.6×150 mm) 3 μm, 35% methanol, Flow rate: 3 g/min.

### 3-(3-Cyano-4-fluorophenyl)-1-(8-fluoro-3,3-dioxido-6-oxo-1,4,5,6-tetrahydro-2H-thiopyrano[3,4-c]isoquinolin-1-yl)-1-methylurea (Compound 215)

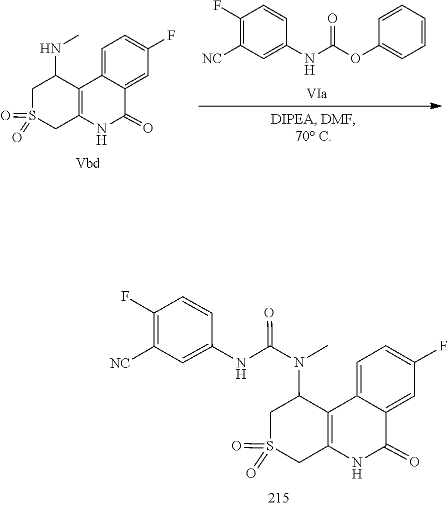 [SMILES: CNC1CS(=O)(=O)Cc2[nH]c(=O)c3cc(F)ccc3c21 ; N#Cc1cc(NC(=O)Oc2ccccc2)ccc1F ; CN(C(=O)Nc1ccc(F)c(C#N)c1)C1CS(=O)(=O)Cc2[nH]c(=O)c3cc(F)ccc3c21]

Racemic 3-(3-cyano-4-fluorophenyl)-1-(8-fluoro-3,3-dioxido-6-oxo-1,4,5,6-tetrahydro-2H-thiopyrano[3,4-c]isoquinolin-1-yl)-1-methylurea was synthesized from 8-fluoro-1-(methylamino)-1,5-dihydro-2H-thiopyrano[3,4-c]isoquinolin-6(4H)-one 3,3-dioxide (Vbd) and phenyl (3-cyano-4-fluorophenyl)carbamate (VIa) in an analogous manner as described above. LCMS: m/z found 459.2 [M+H]⁺, RT=6.10 min, (Method A); ¹H NMR (400 MHz, DMSO-d₆): δ 11.61 (br s, 1H), 8.82 (brs, 1H), 8.11-8.09 (m, 1H) 7.94-7.86 (m, 2H), 7.77-7.72 (m, 1H), 7.53-7.45 (m, 2H), 6.09 (t, 1H), 4.72 (d, 1H), 4.22-4.17 (m, 1H), 3.84-3.80 (m, 1H), 3.64-3.58 (m, 1H), 2.63 (s, 3H).

### 8,9-Difluoro-1-(methylamino)-1,5-dihydro-2H-thiopyrano[3,4-c]isoquinolin-6(4H)-one 3,3-dioxide (Vbe)

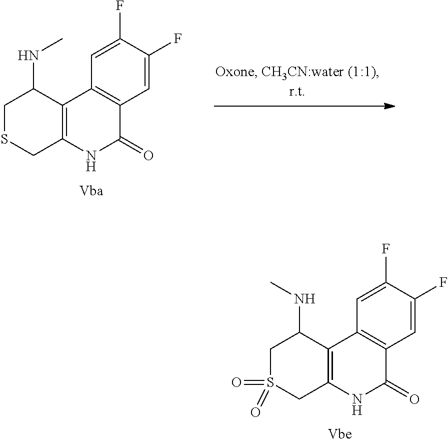 [SMILES: CNC1CSCc2[nH]c(=O)c3cc(F)c(F)cc3c21 ; CNC1CS(=O)(=O)Cc2[nH]c(=O)c3cc(F)c(F)cc3c21]

Racemic 8,9-difluoro-1-(methylamino)-1,5-dihydro-2H-thiopyrano[3,4-c]isoquinolin-6(4H)-one 3,3-dioxide was synthesized from 8-8,9-difluoro-1-(methylamino)-1,5-dihydro-2H-thiopyrano[3,4-c]isoquinolin-6(4H)-one (Vba) and oxone in an analogous manner as described above. LCMS: m/z found 315.24 [M+H]⁺.

### 3-(3-Chloro-4-fluorophenyl)-1-(8,9-difluoro-3,3-dioxido-6-oxo-1,4,5,6-tetrahydro-2H-thiopyrano[3,4-c]isoquinolin-1-yl)-1-methylurea (Compounds 203 and 204)

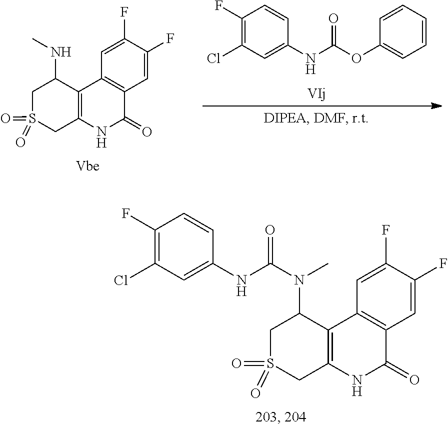 [SMILES: CNC1CS(=O)(=O)Cc2[nH]c(=O)c3cc(F)c(F)cc3c21 ; O=C(Nc1ccc(F)c(Cl)c1)Oc1ccccc1 ; CN(C(=O)Nc1ccc(F)c(Cl)c1)C1CS(=O)(=O)Cc2[nH]c(=O)c3cc(F)c(F)cc3c21]

Racemic 3-(3-chloro-4-fluorophenyl)-1-(8,9-difluoro-3,3-dioxido-6-oxo-1,4,5,6-tetrahydro-2H-thiopyrano[3,4-c]isoquinolin-1-yl)-1-methylurea was synthesized from 8,9-difluoro-1-(methylamino)-1,5-dihydro-2H-thiopyrano[3,4-c]isoquinolin-6(4H)-one 3,3-dioxide (Vbe) and phenyl (3-chloro-4-fluorophenyl)carbamate (VIj) in an analogous manner as described above. The enantiomers were subsequently separated by preparative SFC: Method isocratic, Mobile phase MeOH:CO₂—40:60. Column: Chiralpak-IA (30×250 mm), 5μ, flow rate: 110 g/min.

Enantiomer I (Compound 203): LCMS: m/z found 486.1/488.1 [M+H]⁺, RT=6.09 min, (Method A); ¹H NMR (400 MHz, DMSO-d₆): δ 11.78 (br s, 1H), 8.67 (s, 1H), 8.17-8.12 (m, 1H) 7.85-7.83 (m, 1H), 7.53-7.49 (m, 1H), 7.41-7.33 (m, 2H), 6.09-6.02 (m, 1H), 4.79 (d, 1H), 4.15 (dd, 1H), 3.85-3.81 (m, 1H), 3.64-3.58 (m, 1H), 2.64 (s, 3H); Chiral analytical SFC: RT=1.64 min, Column: Chiralpak IA-3 (4.6×150 mm) 3 μm, 40% methanol, Flow rate: 3 g/min.

Enantiomer II (Compound 204): LCMS: m/z found 486.1/488.1 [M+H]⁺, RT=6.09 (Method A); ¹H NMR (400 MHz, DMSO-d₆): δ 11.78 (br s, 1H), 8.67 (s, 1H), 8.17-8.12 (m, 1H) 7.85-7.83 (m, 1H), 7.53-7.49 (m, 1H), 7.41-7.33 (m, 2H), 6.09-6.02 (m, 1H), 4.79 (d, 1H), 4.15 (dd, 1H), 3.85-3.81 (m, 1H), 3.64-3.58 (m, 1H), 2.64 (s, 3H); Chiral analytical SFC: RT=3.56 min, Column: Chiralpak IA-3 (4.6×150 mm) 3 μm, 40% methanol, Flow rate: 3 g/min.

### 3-(3-Cyano-4-fluorophenyl)-1-(8,9-difluoro-3,3-dioxido-6-oxo-1,4,5,6-tetrahydro-2H-thiopyrano[3,4-c]isoquinolin-1-yl)-1-methylurea (Compounds 205 and 206)

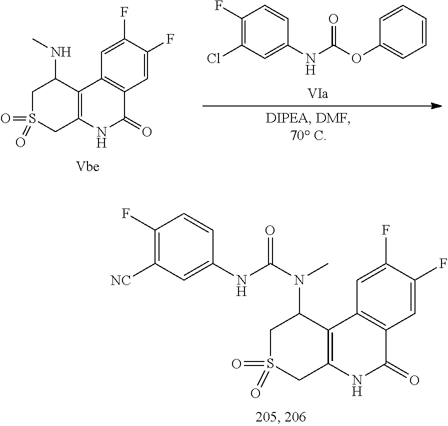 [SMILES: CNC1CS(=O)(=O)Cc2[nH]c(=O)c3cc(F)c(F)cc3c21 ; O=C(Nc1ccc(F)c(Cl)c1)Oc1ccccc1 ; CN(C(=O)Nc1ccc(F)c(C#N)c1)C1CS(=O)(=O)Cc2[nH]c(=O)c3cc(F)c(F)cc3c21]

Racemic 3-(3-cyano-4-fluorophenyl)-1-(8,9-difluoro-3,3-dioxido-6-oxo-1,4,5,6-tetrahydro-2H-thiopyrano[3,4-c]isoquinolin-1-yl)-1-methylurea was synthesized from 8,9-difluoro-1-(methylamino)-1,5-dihydro-2H-thiopyrano[3,4-c]isoquinolin-6(4H)-one 3,3-dioxide (Vbe) and phenyl (3-cyano-4-fluorophenyl)carbamate (VIa) in an analogous manner as described above. The enantiomers were subsequently separated by preparative SFC: Method isocratic, Mobile phase MeOH:CO₂—50:50. Column: Chiralpak-IA (30×250 mm), 5μ, flow rate: 110 g/min.

Enantiomer I (Compound 205): LCMS: m/z found 477.1 [M+H]⁺, RT=5.68 min, (Method A); ¹H NMR (400 MHz, DMSO-d₆): δ 11.78 (br s, 1H), 8.85 (s, 1H), 8.17-8.12 (m, 1H) 8.05-8.03 (m, 1H), 7.90-7.86 (m, 1H), 7.49 (t, 1H), 7.39-7.34 (m, 1H), 6.03 (t, 1H), 4.78 (d, 1H), 4.16 (dd, 1H), 3.86-3.82 (m, 1H), 3.65-3.60 (m, 1H), 2.65 (s, 3H); Chiral analytical SFC: RT=1.12 min, Column: Chiralpak IA-3 (4.6×150 mm) 3 μm, 40% methanol, Flow rate: 3 g/min.

Enantiomer II (Compound 206): LCMS: m/z found 477.1 [M+H]⁺, RT=5.68 min, (Method A); ¹H NMR (400 MHz, DMSO-d₆): δ 11.78 (br s, 1H), 8.84 (s, 1H), 8.15-8.11 (m, 1H) 8.05-8.03 (m, 1H), 7.91-7.86 (m, 1H), 7.49 (t, 1H), 7.37-7.32 (m, 1H), 6.03 (t, 1H), 4.75 (d, 1H), 4.16 (dd, 1H), 3.83-3.80 (m, 1H), 3.64-3.59 (m, 1H), 2.65 (s, 3H); Chiral analytical SFC: RT=3.90 min, Column: Chiralpak IA-3 (4.6×150 mm) 3 μm, 40% methanol, Flow rate: 3 g/min.

### Example 2: Biological Results

Representative compounds of the disclosure were tested for their abilities to inhibit formation of relaxed circular DNA (rcDNA) in a HepDE19 assay, as described elsewhere herein. Results are illustrated in Table 3.

TABLE 3

| | | DE- |
|---|---|---|
| | | 19bDNA |
| No. | Nomenclature | EC₅₀ (uM) |
| 1 | 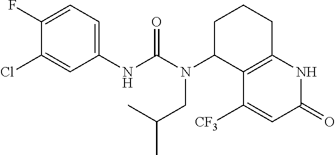 [SMILES: CC(C)CN(C(=O)Nc1ccc(F)c(Cl)c1)C1CCCc2[nH]c(=O)cc(C(F)(F)F)c21] 3-(3-chloro-4-fluorophenyl)-1-isobutyl-1-(2-oxo-4- (trifluoromethyl)-1,2,5,6,7,8-hexahydroquinolin-5-yl)urea | 0.52 |
| 2 | 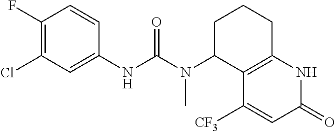 [SMILES: CN(C(=O)Nc1ccc(F)c(Cl)c1)C1CCCc2[nH]c(=O)cc(C(F)(F)F)c21] 3-(3-chloro-4-fluorophenyl)-1-methyl-1-(2-oxo-4- (trifluoromethyl)-1,2,5,6,7,8-hexahydroquinolin-5-yl)urea | 1.5 |
| 3 | 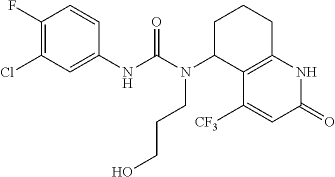 [SMILES: O=C(Nc1ccc(F)c(Cl)c1)N(CCCO)C1CCCc2[nH]c(=O)cc(C(F)(F)F)c21] 3-(3-chloro-4-fluorophenyl)-1-(3-hydroxypropyl)-1- (2-oxo-4-(trifluoromethyl)-1,2,5,6,7,8- hexahydroquinolin-5-yl)urea | 1.7 |
| 4 | 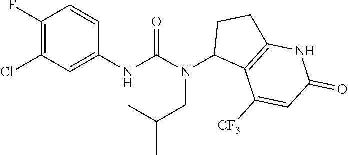 [SMILES: CC(C)CN(C(=O)Nc1ccc(F)c(Cl)c1)C1CCc2[nH]c(=O)cc(C(F)(F)F)c21] 3-(3-chloro-4-fluorophenyl)-1-isobutyl-1-(2-oxo-4- (trifluoromethyl)-2,5,6,7-tetrahydro-1H-cyclopenta[b] pyridin-5-yl)urea | 0.91 |
| 5 | 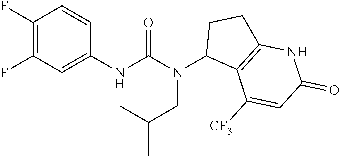 [SMILES: CC(C)CN(C(=O)Nc1ccc(F)c(F)c1)C1CCc2[nH]c(=O)cc(C(F)(F)F)c21] 3-(3,4-difluorophenyl)-1-isobutyl-1-(2-oxo-4- (trifluoromethyl)-2,5,6,7-tetrahydro-1H-cyclopenta[b] pyridin-5-yl)urea | 1.1 |
| 6 | 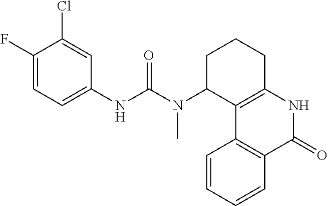 [SMILES: CN(C(=O)Nc1ccc(F)c(Cl)c1)C1CCCc2[nH]c(=O)c3ccccc3c21] 3-(3-chloro-4-fluorophenyl)-1-methyl-1-(6-oxo- 1,2,3,4,5,6-hexahydrophenanthridin-1-yl)urea | 0.08 |
| 7 | 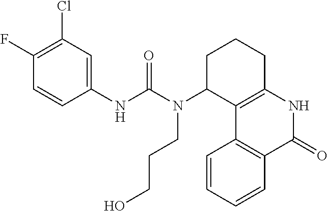 [SMILES: O=C(Nc1ccc(F)c(Cl)c1)N(CCCO)C1CCCc2[nH]c(=O)c3ccccc3c21] 3-(3-chloro-4-fluorophenyl)-1-(3-hydroxypropyl)-1-(6- oxo-1,2,3,4,5,6-hexahydrophenanthridin-1-yl)urea | 0.75 |
| 8 | 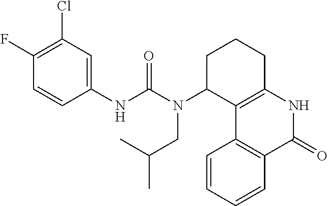 [SMILES: CC(C)CN(C(=O)Nc1ccc(F)c(Cl)c1)C1CCCc2[nH]c(=O)c3ccccc3c21] 3-(3-chloro-4-fluorophenyl)-1-isobutyl-1-(6-oxo- 1,2,3,4,5,6-hexahydrophenanthridin-1-yl)urea | 0.47 |
| 9 | 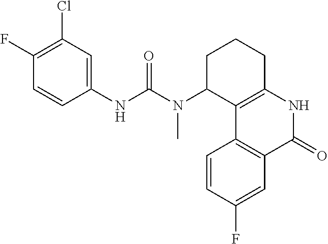 [SMILES: CN(C(=O)Nc1ccc(F)c(Cl)c1)C1CCCc2[nH]c(=O)c3cc(F)ccc3c21] 3-(3-chloro-4-fluorophenyl)-1-(8-fluoro-6-oxo- 1,2,3,4,5,6-hexahydrophenanthridin-1-yl)-1-methylurea | 0.03 |
| 10 | 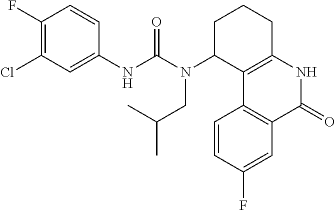 [SMILES: CC(C)CN(C(=O)Nc1ccc(F)c(Cl)c1)C1CCCc2[nH]c(=O)c3cc(F)ccc3c21] 3-(3-chloro-4-fluorophenyl)-1-(8-fluoro-6-oxo- 1,2,3,4,5,6-hexahydrophenanthridin-1-yl)-1-isobutylurea | 0.06 |
| 11 | 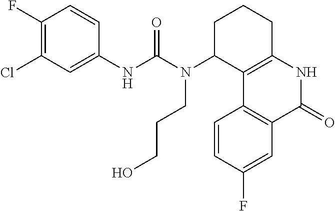 [SMILES: O=C(Nc1ccc(F)c(Cl)c1)N(CCCO)C1CCCc2[nH]c(=O)c3cc(F)ccc3c21] 3-(3-chloro-4-fluorophenyl)-1-(8-fluoro-6-oxo- 1,2,3,4,5,6-hexahydrophcnanthridin-1-yl)-1-(3- hydroxypropyl)urea | 0.06 |
| 12 | 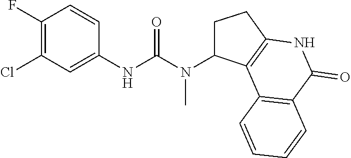 [SMILES: CN(C(=O)Nc1ccc(F)c(Cl)c1)C1CCc2[nH]c(=O)c3ccccc3c21] 3-(3-chloro-4-fluorophenyl)-1-methyl-1-(5-oxo-2,3,4,5- tetrahydro-1H-cyclopenta[c]isoquinolin-1-yl)urea | 0.09 |
| 13 | 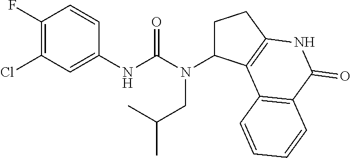 [SMILES: CC(C)CN(C(=O)Nc1ccc(F)c(Cl)c1)C1CCc2[nH]c(=O)c3ccccc3c21] 3-(3-chloro-4-fluorophenyl)-1-isobutyl-1-(5-oxo-2,3,4,5 tetrahydro-1H-cyclopenta[c]isoquinolin-1-yl)urea | 1.3 |
| 14 | 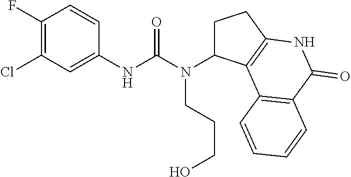 [SMILES: O=C(Nc1ccc(F)c(Cl)c1)N(CCCO)C1CCc2[nH]c(=O)c3ccccc3c21] 3-(3-chloro-4-fluorophenyl)-1-isobutyl-1-(5-oxo-2,3,4,5- tetrahydro-1H-cyclopenta[c]isoquinolin-1-yl)urea | 0.10 |
| 15 | 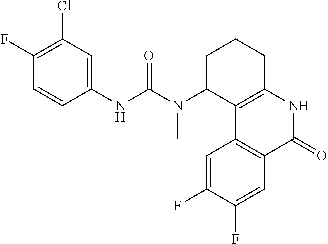 [SMILES: CN(C(=O)Nc1ccc(F)c(Cl)c1)C1CCCc2[nH]c(=O)c3cc(F)c(F)cc3c21] 3-(3-chloro-4-fluorophenyl)-1-(8,9-difluoro-6-oxo- 1,2,3,4,5,6-hexahydrophenanthridin-1-yl)-1-methylurea | 0.02 |
| 16 | 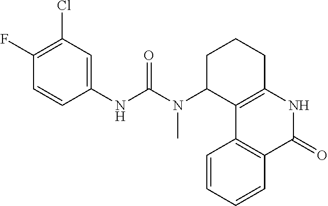 [SMILES: CN(C(=O)Nc1ccc(F)c(Cl)c1)C1CCCc2[nH]c(=O)c3ccccc3c21] 3-(3-chloro-4-fluorophenyl)-1-methyl-1-(6-oxo- 1,2,3,4,5,6-hexahydrophenanthridin-1-yl)urea; Enantiomer I | 0.76 |
| 17 | 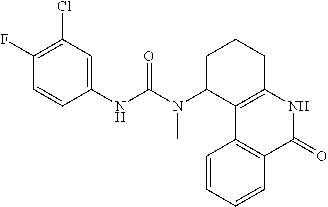 [SMILES: CN(C(=O)Nc1ccc(F)c(Cl)c1)C1CCCc2[nH]c(=O)c3ccccc3c21] 3-(3-chloro-4-fluorophenyl)-1-methyl-1-(6-oxo- 1,2,3,4,5,6-hexahydrophenanthridin-1-yl)urea; Enantiomer II | 0.05 |
| 18 | 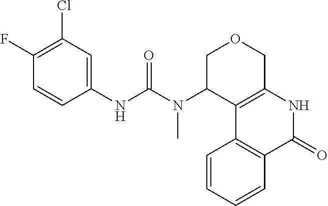 [SMILES: CN(C(=O)Nc1ccc(F)c(Cl)c1)C1COCc2[nH]c(=O)c3ccccc3c21] 3-(3-chloro-4-fluorophenyl)-1-methyl-1-(6-oxo-1,4,5,6- tetrahydro-2H-pyrano[3,4-c]isoquinolin-1-yl)urea | 0.03 |
| 19 | 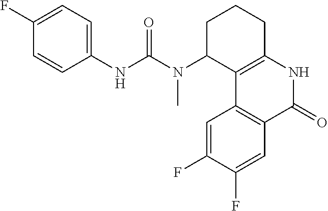 [SMILES: CN(C(=O)Nc1ccc(F)cc1)C1CCCc2[nH]c(=O)c3cc(F)c(F)cc3c21] 1-(8,9-difluoro-6-oxo-1,2,3,4,5,6-hexahydrophenanthridin- 1-yl)-3-(4-fluorophenyl)-1-methylurea | 0.16 |
| 20 | 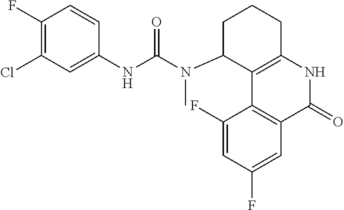 [SMILES: CN(C(=O)Nc1ccc(F)c(Cl)c1)C1CCCc2[nH]c(=O)c3cc(F)cc(F)c3c21] 3-(3-chloro-4-fluorophenyl)-1-(8,10-difluoro-6-oxo- 1,2,3,4,5,6-hexahydrophenanthridin-1-yl)-1-methylurea | 0.51 |
| 21 | 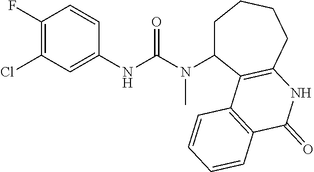 [SMILES: CN(C(=O)Nc1ccc(F)c(Cl)c1)C1CCCCc2[nH]c(=O)c3ccccc3c21] 3-(3-chloro-4-fluorophenyl)-1-methyl-1-(5-oxo- 6,7,8,9,10,11-hexahydro-5H-cyclohepta[c]isoquinolin- 11-yl)urea | 0.15 |
| 22 | 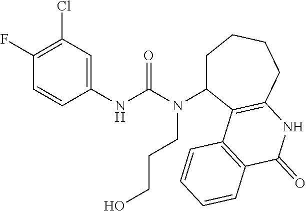 [SMILES: O=C(Nc1ccc(F)c(Cl)c1)N(CCCO)C1CCCCc2[nH]c(=O)c3ccccc3c21] 3-(3-chloro-4-fluorophenyl)-1-(3-hydroxypropyl)-1- (5-oxo-6,7,8,9,10,11-hexahydro-5H-cyclohepta[c] isoquinolin-l l-yl)ure | 0.60 |
| 23 | 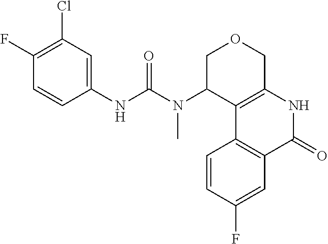 [SMILES: CN(C(=O)Nc1ccc(F)c(Cl)c1)C1COCc2[nH]c(=O)c3cc(F)ccc3c21] 3-(3-chloro-4-fluorophenyl)-1-(8-fluoro-6-oxo-1,4,5,6- tetrahydro-2H-pyrano[3,4-c]isoquinolin-1-yl)-1- methylurea | 0.02 |
| 24 | 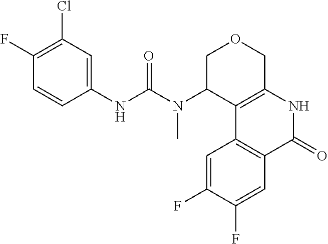 [SMILES: CN(C(=O)Nc1ccc(F)c(Cl)c1)C1COCc2[nH]c(=O)c3cc(F)c(F)cc3c21] 3-(3-chloro-4-fluorophenyl)-1-(8,9-difluoro-6-oxo- 1,4,5,6-tetrahydro-2H-pyrano[3,4-c]isoquinolin-1-yl)-1- methylurea | 0.01 |
| 25 | 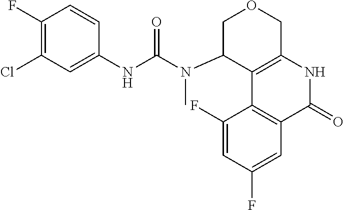 [SMILES: CN(C(=O)Nc1ccc(F)c(Cl)c1)C1COCc2[nH]c(=O)c3cc(F)cc(F)c3c21] 3-(3-chloro-4-fluorophenyl)-1-(8,10-difluoro-6-oxo- 1,4,5,6-tetrahydro-2H-pyrano[3,4-c]isoquinolin- 1-yl)-l-methylurea | 0.29 |
| 26 | 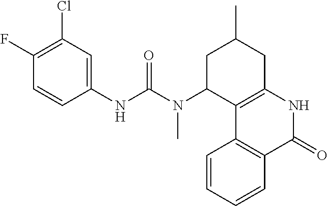 [SMILES: CC1Cc2[nH]c(=O)c3ccccc3c2C(N(C)C(=O)Nc2ccc(F)c(Cl)c2)C1] 3-(3-chloro-4-fluorophenyl)-1-methyl-1-(3-methyl-6- oxo-1,2,3,4,5,6-hexahydrophenanthridin-1-yl)urea 85% racemic cis: 15% racemic trans | 2.9 |
| 27 | 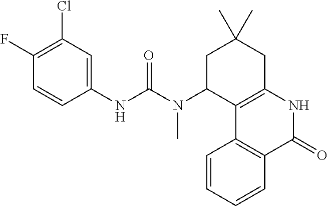 [SMILES: CN(C(=O)Nc1ccc(F)c(Cl)c1)C1CC(C)(C)Cc2[nH]c(=O)c3ccccc3c21] 3-(3-chloro-4-fluorophenyl)-1-(3,3-dimethyl-6-oxo- 1,2,3,4,5,6-hexahydrophenanthridin-1-yl)-1-methylurea | 12 |
| 28 | 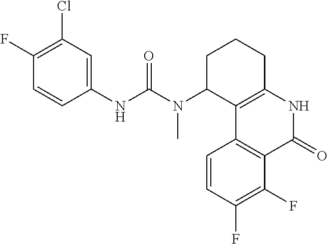 [SMILES: CN(C(=O)Nc1ccc(F)c(Cl)c1)C1CCCc2[nH]c(=O)c3c(F)c(F)ccc3c21] 3-(3-chloro-4-fluorophenyl)-1-(7,8-difluoro-6-oxo- 1,2,3,4,5,6-hexahydrophenanlhridin-1-yl)-1-methylurea | 0.44 |
| 29 | 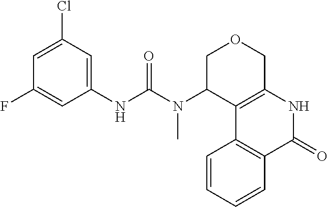 [SMILES: CN(C(=O)Nc1cc(F)cc(Cl)c1)C1COCc2[nH]c(=O)c3ccccc3c21] 3-(3-chloro-5-fluorophenyl)-1-methyl-1-(6-oxo-1,4,5,6- tetrahydro-2H-pyrano[3,4-c]isoquinolin-1-yl)urea | 0.12 |
| 30 | 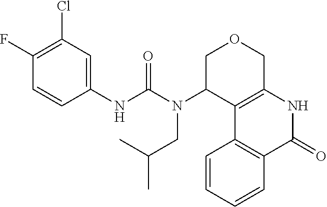 [SMILES: CC(C)CN(C(=O)Nc1ccc(F)c(Cl)c1)C1COCc2[nH]c(=O)c3ccccc3c21] 3-(3-chloro-4-lluorophenyl)-1-isobutyl-1-(6-oxo-1,4,5,6- tetrahydro-2H-pyrano[3,4-c]isoquinolin-1-yl)urea | 0.02 |
| 31 | 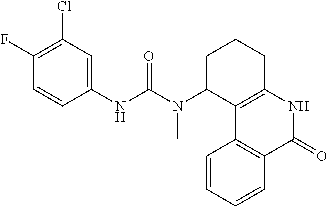 [SMILES: CN(C(=O)Nc1ccc(F)c(Cl)c1)C1CCCc2[nH]c(=O)c3ccccc3c21] 3-(3-chloro-4-fluorophenyl)-1-methyl-1-(5-methyl-6-oxo- 1,2,3,4,5,6-hexahydrophenanthridin-1-yl)urea | 0.26 |
| 32 | 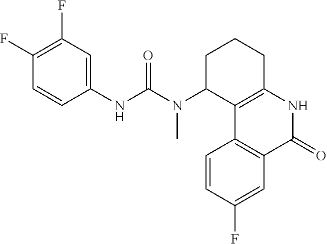 [SMILES: CN(C(=O)Nc1ccc(F)c(F)c1)C1CCCc2[nH]c(=O)c3cc(F)ccc3c21] 3-(3,4-difluorophenyl)-1-(8-fluoro-6-oxo-1,2,3,4,5,6- hexahydrophenamhridin-1-yl)-1-methylurea | 0.11 |
| 33 | 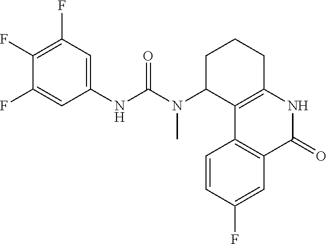 [SMILES: CN(C(=O)Nc1cc(F)c(F)c(F)c1)C1CCCc2[nH]c(=O)c3cc(F)ccc3c21] 1-(8-fluoro-6-oxo-1,2,3,4,5,6-hexahydrophenanthridin- 1-yl)-1-methyl-3-(3,4,5-trifluorophenyl)urea | 0.10 |
| 34 | 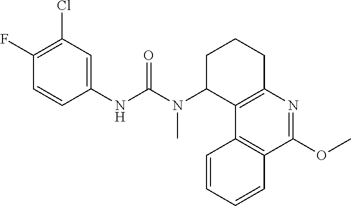 [SMILES: COc1nc2c(c3ccccc13)C(N(C)C(=O)Nc1ccc(F)c(Cl)c1)CCC2] 3-(3-chloro-4-fluorophenyl)-1-(6-methoxy-1,2,3,4- tetrahydrophenanthridin-1-yl)-1-methylurea | 1.8 |
| 35 | 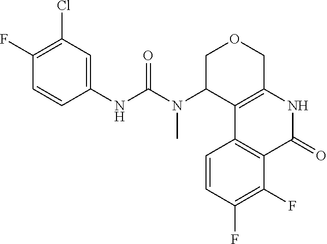 [SMILES: CN(C(=O)Nc1ccc(F)c(Cl)c1)C1COCc2[nH]c(=O)c3c(F)c(F)ccc3c21] 3-(3-chloro-4-fluorophenyl)-1-(7,8-difluoro-6-oxo- 1,4,5,6-tetrahydro-2H-pyrano[3,4-c]isoquinolin-1-yl)- 1-methylurea | 0.19 |
| 36 | 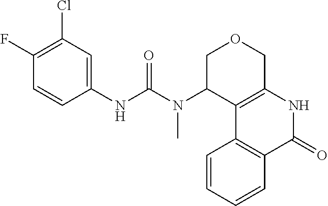 [SMILES: CN(C(=O)Nc1ccc(F)c(Cl)c1)C1COCc2[nH]c(=O)c3ccccc3c21] 3-(3-chloro-4-fluorophenyl)-1-methyl-1-(6-oxo-1,4,5,6- tetrahydro-2H-pyrano[3,4-c]isoquinolin-1-yl)urea; Enantiomer I | 4.6 |
| 37 | 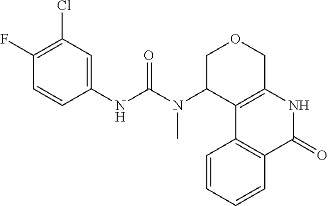 [SMILES: CN(C(=O)Nc1ccc(F)c(Cl)c1)C1COCc2[nH]c(=O)c3ccccc3c21] 3-(3-chloro-4-fluorophenyl)-1-methyl-1-(6-oxo-1,4,5,6- tetrahydro-2H-pyrano[3,4-c]isoquinolin-l-yl)urea; Enantiomer II | 0.03 |
| 38 | 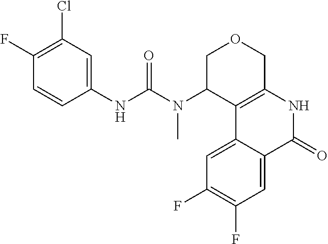 [SMILES: CN(C(=O)Nc1ccc(F)c(Cl)c1)C1COCc2[nH]c(=O)c3cc(F)c(F)cc3c21] 3-(3-chloro-4-fluorophenyl)-1-(8,9-difluoro-6-oxo- 1,2,3,4,5,6-hexahydrophenanthridin-1-yl)-1-methylurea; Enantiomer I | 1.0 |
| 39 | 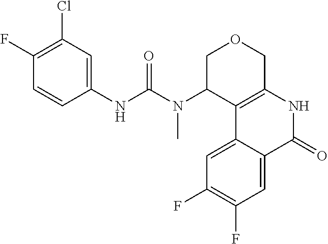 [SMILES: CN(C(=O)Nc1ccc(F)c(Cl)c1)C1COCc2[nH]c(=O)c3cc(F)c(F)cc3c21] 3-(3-chloro-4-fluorophenyl)-1-(8,9-difluoro-6-oxo- 1,2,3,4,5,6-hexahydrophenanthridin-1-yl)-1-methylurea; Enantiomer II | 0.01 |
| 40 | 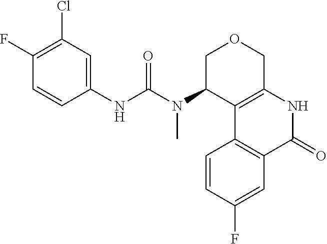 [SMILES: CN(C(=O)Nc1ccc(F)c(Cl)c1)[C@H]1COCc2[nH]c(=O)c3cc(F)ccc3c21] (R)-3-(3-chloro-4-fluorophenyl)-1-(8-fluoro-6-oxo- 1,4,5,6-tetrahydro-2H-pyrano[3,4-c]isoquinolin-1-yl)- 1-methylurea; Enantiomer I | 1.0 |
| 41 | 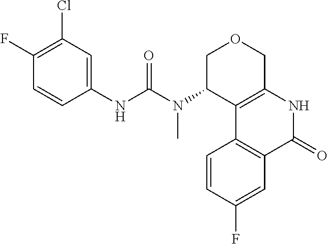 [SMILES: CN(C(=O)Nc1ccc(F)c(Cl)c1)[C@@H]1COCc2[nH]c(=O)c3cc(F)ccc3c21] (S)-3-(3-Chloro-4-fluorophenyl)-1-(8-fluoro-6-oxo- 1,4,5,6- tetrahydro-2H-pyrano[3,4-c]isoquinolin-1-yl)- 1-methylurea; Enantiomer II | 0.01 |
| 43 | 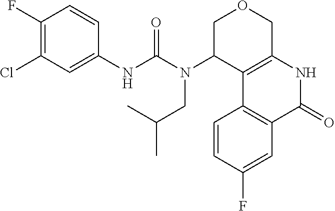 [SMILES: CC(C)CN(C(=O)Nc1ccc(F)c(Cl)c1)C1COCc2[nH]c(=O)c3cc(F)ccc3c21] 3-(3-chloro-4-fluorophenyl)-1-(8-fluoro-6-oxo-1,4,5,6- tetrahydro-2H-pyrano[3,4-c]isoquinolin-1-yl)-1- isobutylurea | 0.01 |
| 44 | 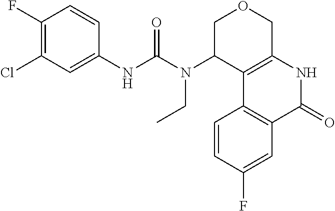 [SMILES: CCN(C(=O)Nc1ccc(F)c(Cl)c1)C1COCc2[nH]c(=O)c3cc(F)ccc3c21] 3-(3-chloro-4-fluorophenyl)-1-ethyl-1-(8-fluoro-6-oxo- 1,4,5,6-tetrahydro-2H-pyrano[3,4-c]isoquinolin-1-yl)urea | 0.01 |
| 45 | 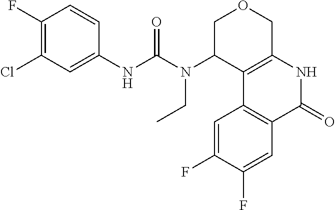 [SMILES: CCN(C(=O)Nc1ccc(F)c(Cl)c1)C1COCc2[nH]c(=O)c3cc(F)c(F)cc3c21] 3-(3-chloro-4-fluorophenyl)-1-(8,9-difluoro-6-oxo-1,4,5,6- tetrahydro-2H-pyrano[3,4-c]isoquinolin-1-yl)-1-ethylurea | 0.01 |
| 46 | 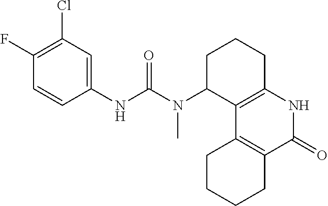 [SMILES: CN(C(=O)Nc1ccc(F)c(Cl)c1)C1CCCc2[nH]c(=O)c3c(c21)CCCC3] 3-(3-chloro-4-fluorophenyl)-1-methyl-1-(6-oxo- 1,2,3,4,5,6,7,8,9,10-decahydrophenanthridin-1-yl)urea; Enantiomer I | 1.2 |
| 47 | 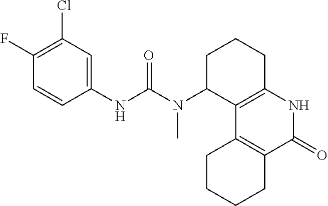 [SMILES: CN(C(=O)Nc1ccc(F)c(Cl)c1)C1CCCc2[nH]c(=O)c3c(c21)CCCC3] 3-(3-chloro-4-fluorophenyl)-1-methyl-1-(6-oxo- 1,2,3,4,5,6,7,8,9,10-decahydrophenanthridin-1-yl)urea; Enantiomer II | 0.06 |
| 48 | 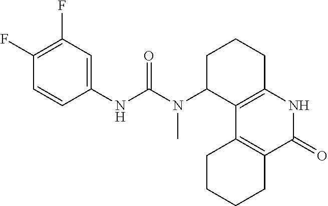 [SMILES: CN(C(=O)Nc1ccc(F)c(F)c1)C1CCCc2[nH]c(=O)c3c(c21)CCCC3] 3-(3,4-difluorophenyl)-1-methyl-1-(6-oxo- 1,2,3,4,5,6,7,8,9,10-decahydrophenanthridin-1-yl)urea; Enantiomer I | 3.1 |
| 49 | 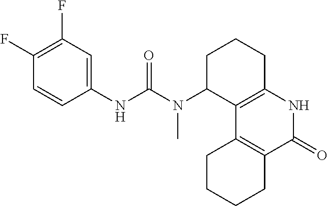 [SMILES: CN(C(=O)Nc1ccc(F)c(F)c1)C1CCCc2[nH]c(=O)c3c(c21)CCCC3] 3-(3,4-difluorophenyl)-1-methyl-1-(6-oxo- 1,2,3,4,5,6,7,8,9,10-decahydrophenanthridin-1-yl)urea; Enantiomer II | 0.16 |
| 50 | 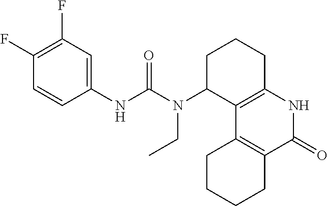 [SMILES: CCN(C(=O)Nc1ccc(F)c(F)c1)C1CCCc2[nH]c(=O)c3c(c21)CCCC3] 3-(3,4-difluorophenyl)-1-ethyl-1-(6-oxo- 1,2,3,4,5,6,7,8,9,10-decahydrophenanthridin-1-yl)urea; Enantiomer I | 6.8 |
| 51 | 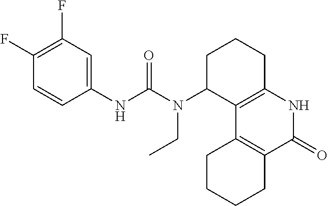 [SMILES: CCN(C(=O)Nc1ccc(F)c(F)c1)C1CCCc2[nH]c(=O)c3c(c21)CCCC3] 3-(3,4-difluorophenyl)-1-ethyl-1-(6-oxo- 1,2,3,4,5,6,7,8,9,10-decahydrophenanthridin-1-yl)urea; Enantiomer II | 0.10 |
| 53 | 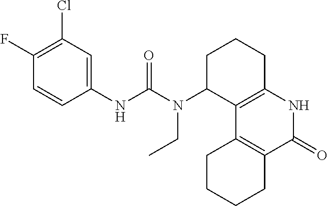 [SMILES: CCN(C(=O)Nc1ccc(F)c(Cl)c1)C1CCCc2[nH]c(=O)c3c(c21)CCCC3] 3-(3-chloro-4-fluorophenyl)-1-ethyl-1-(6-oxo- 1,2,3,4,5,6,7,8,9,10-decahydrophenanthridin-1-yl)urea; Enantiomer I | 2.6 |
| 54 | 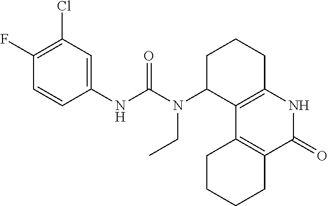 [SMILES: CCN(C(=O)Nc1ccc(F)c(Cl)c1)C1CCCc2[nH]c(=O)c3c(c21)CCCC3] 3-(3-chloro-4-fluorophenyl)-1-ethyl-1-(6-oxo- 1,2,3,4,5,6,7,8,9,10-decahydrophenanthridin-1-yl)urea; Enantiomer II | 0.03 |
| 55 | 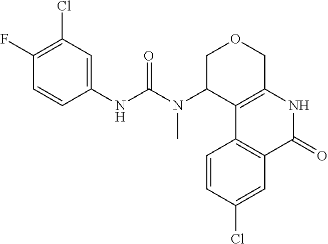 [SMILES: CN(C(=O)Nc1ccc(F)c(Cl)c1)C1COCc2[nH]c(=O)c3cc(Cl)ccc3c21] 3-(3-chloro-4-fluorophenyl)-1-(8-chloro-6-oxo-1,4,5,6- tetrahydro-2H-pyrano[3,4-c]isoquinolin-1-yl)-1- methylurea | 0.05 |
| 56 | 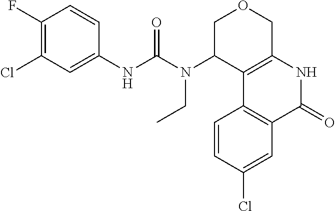 [SMILES: CCN(C(=O)Nc1ccc(F)c(Cl)c1)C1COCc2[nH]c(=O)c3cc(Cl)ccc3c21] 3-(3-chloro-4-fluorophenyl)-1-(8-chloro-6-oxo-1,4,5,6- tetrahydro-2H-pyrano[3,4-c]isoquinolin-1-yl)-1-ethylurea | 0.05 |
| 57 | 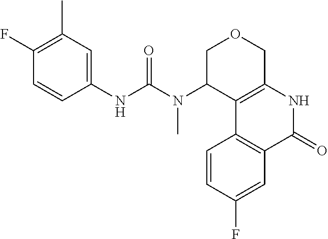 [SMILES: Cc1cc(NC(=O)N(C)C2COCc3[nH]c(=O)c4cc(F)ccc4c32)ccc1F] 3-(4-fluoro-3-methylphenyl)-1-(8-fluoro-6-oxo-1,4,5,6- tetrahydro-2H-pyrano[3,4-c]isofluinolin-1-yl)-1- methylurea | 0.01 |
| 58 | 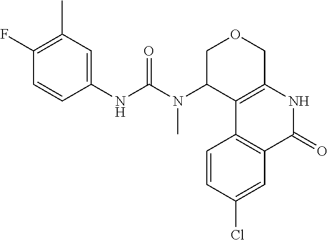 [SMILES: Cc1cc(NC(=O)N(C)C2COCc3[nH]c(=O)c4cc(Cl)ccc4c32)ccc1F] 1-(8-chloro-6-oxo-1,4,5,6-tetrahydro-2H-pyrano[3,4-c] isoquinolin-1-yl)-3-(4-fluoro-3-methylphenyl)-1- methylurea | 0.04 |
| 59 | 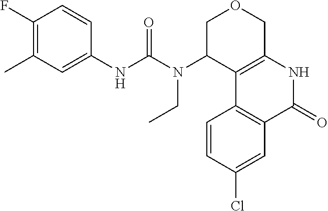 [SMILES: CCN(C(=O)Nc1ccc(F)c(C)c1)C1COCc2[nH]c(=O)c3cc(Cl)ccc3c21] 1-(8-chloro-6-oxo-1,4,5,6-tetrahydro-2H-pyrano[3,4-c] isoquinolin-1-yl)-1-elhyl-3-(4-fluoro-3-methylphenyl)-1- methylurea | 0.02 |
| 60 | 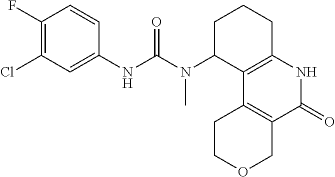 [SMILES: CN(C(=O)Nc1ccc(F)c(Cl)c1)C1CCCc2[nH]c(=O)c3c(c21)CCOC3] 3-(3-chloro-4-fluorophenyl)-1-methyl-1-(5-oxo- 1,4,5,6,7,8,9,10-octahydro-2H-pyrano[3,4-c]quinolin- 10-yl)urea; Enantiomer I | 3.2 |
| 61 | 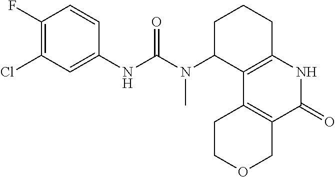 [SMILES: CN(C(=O)Nc1ccc(F)c(Cl)c1)C1CCCc2[nH]c(=O)c3c(c21)CCOC3] 3-(3-chloro-4-fluorophenyl)-1-methyl-1-(5-oxo- 1,4,5,6,7,8,9,10-octahydro-2H-pyrano[3,4-c]quinolin- 10-yl)urea; Enantiomer II | 5.1 |
| 62 | 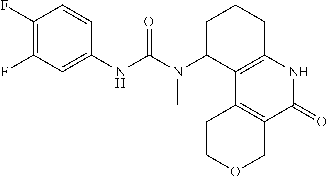 [SMILES: CN(C(=O)Nc1ccc(F)c(F)c1)C1CCCc2[nH]c(=O)c3c(c21)CCOC3] 3-(3,4-difluorophenyl)-1-methyl-1-(5-oxo- 1,4,5,6,7,8,9,10-octahydro-2H-pyrano[3,4-c]quinolin- 10-yl)urea; Enantiomer I | 1.3 |
| 63 | 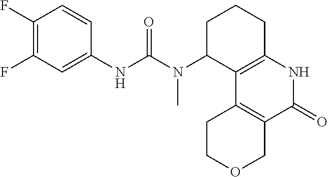 [SMILES: CN(C(=O)Nc1ccc(F)c(F)c1)C1CCCc2[nH]c(=O)c3c(c21)CCOC3] 3-(3,4-difluorophenyl)-1-methyl-1-(5-oxo- 1,4,5,6,7,8,9,10-octahydro-2H-pyrano[3,4-c]quinolin- 10-yl)urea; Enantiomer II | 3.1 |
| 64 | 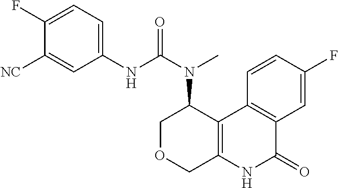 [SMILES: CN(C(=O)Nc1ccc(F)c(C#N)c1)[C@@H]1COCc2[nH]c(=O)c3cc(F)ccc3c21] (S)-3-(3-cyano-4-fluorophenyl)-1-(8-fluoro-6-oxo-1,4,5,6- tetrahydro-2H-pyrano[3,4-c]isoquinolin-1-yl)-1-methylurea; Single enantiomer | 0.02 |
| 65 | 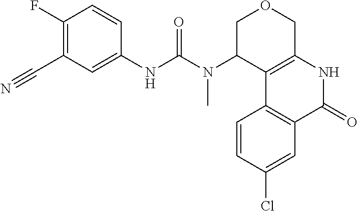 [SMILES: CN(C(=O)Nc1ccc(F)c(C#N)c1)C1COCc2[nH]c(=O)c3cc(Cl)ccc3c21] 1-(8-chloro-6-oxo-1,4,5,6-tetrahydro-2H-pyrano[3,4-c] isoquinolin-1-yl)-3-(3-cyano-4-fluorophenyl)-1-methylurea | 0.08 |
| 66 | 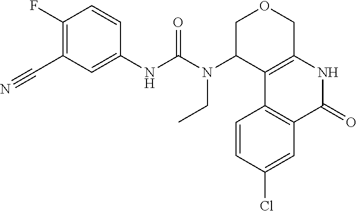 [SMILES: CCN(C(=O)Nc1ccc(F)c(C#N)c1)C1COCc2[nH]c(=O)c3cc(Cl)ccc3c21] 1-(8-chloro-6-oxo-1,4,5,6-tetrahydro-2H-pyrano[3,4-c] isoquinolin-1-yl)-3-(3-cyano-4-fluorophenyl)-1-ethylurea | 0.05 |
| 67 | 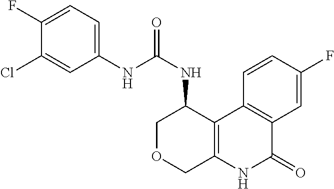 [SMILES: O=C(Nc1ccc(F)c(Cl)c1)N[C@@H]1COCc2[nH]c(=O)c3cc(F)ccc3c21] (S)-1-(3-chloro-4-fluorophenyl)-3-(8-fluoro-6-oxo-1,4,5,6- tetrahydro-2H-pyrano[3,4-c]isoquinolin-1-yl)urea; Single enantiomer | 0.09 |
| 68 | 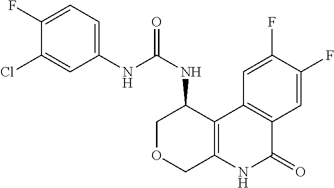 [SMILES: O=C(Nc1ccc(F)c(Cl)c1)N[C@@H]1COCc2[nH]c(=O)c3cc(F)c(F)cc3c21] (S)-1-(3-chloro-4-fluorophenyl)-3-(8,9-difluoro-6-oxo- 1,4,5,6-tetrahydro-2H-pyrano[3,4-c]isoquinolin-1-yl)urea; Single enantiomer | 0.03 |
| 69 | 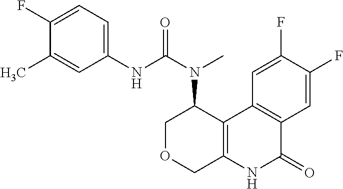 [SMILES: Cc1cc(NC(=O)N(C)[C@@H]2COCc3[nH]c(=O)c4cc(F)c(F)cc4c32)ccc1F] (S)-1-(8,9-difluoro-6-oxo-1,4,5,6-tetrahydro-2H-pyrano [3,4-c]isoquinolin-1-yl)-3-(4-fluoro-3-methylphenyl)-1- methylurea; Single enantiomer | 0.01 |
| 70 | 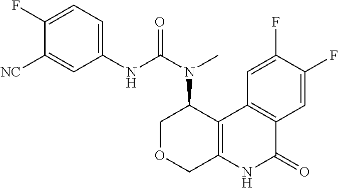 [SMILES: CN(C(=O)Nc1ccc(F)c(C#N)c1)[C@@H]1COCc2[nH]c(=O)c3cc(F)c(F)cc3c21] (S)-3-(3-cyano-4-fluorophenyl)-1-(8,9-difluoro-6-oxo- 1,4,5,6-tetrahydro-2H-pyrano[3,4-c]isoquinolin-1-yl)- 1-methylurea; Enantiomer II | 0.01 |
| 71 | 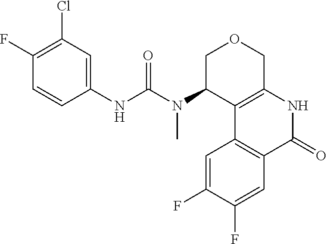 [SMILES: CN(C(=O)Nc1ccc(F)c(Cl)c1)[C@H]1COCc2[nH]c(=O)c3cc(F)c(F)cc3c21] (R)-3-(3-chloro-4-fluorophenyl)-1-(8,9-difluoro-6-oxo- 1,4,5,6-tetrahydro-2H-pyrano[3,4-c]isoquinolin-1-yl)- 1-methylurea; Enantiomer I | 0.21 |
| 72 | 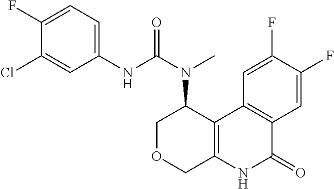 [SMILES: CN(C(=O)Nc1ccc(F)c(Cl)c1)[C@@H]1COCc2[nH]c(=O)c3cc(F)c(F)cc3c21] (S)-3-(3-chloro-4-fluorophenyl)-1-(8,9-difluoro-6-oxo- 1,4,5,6-tetrahydro-2H-pyrano[3,4-c]isoquinolin-1-yl)- 1-methylurea; Enantiomer II | 0.01 |
| 73 | 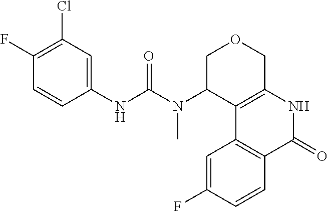 [SMILES: CN(C(=O)Nc1ccc(F)c(Cl)c1)C1COCc2[nH]c(=O)c3ccc(F)cc3c21] 3-(3-chloro-4-fluorophenyl)-1-(9-fluoro-6-oxo-1,4,5,6- tetrahydro-2H-pyrano[3,4-c]isoquinolin-1-yl)-1-methylurea | 0.02 |
| 74 | 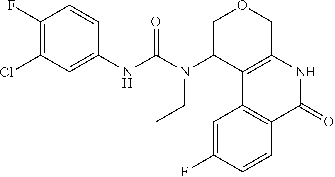 [SMILES: CCN(C(=O)Nc1ccc(F)c(Cl)c1)C1COCc2[nH]c(=O)c3ccc(F)cc3c21] 3-(3-chloro-4-fluorophenyl)-1-ethyl-1-(9-fluoro-6-oxo- 1,4,5,6-tetrahydro-2H-pyrano[3,4-c]isoquinolin-1-yl)urea | 0.02 |
| 75 | 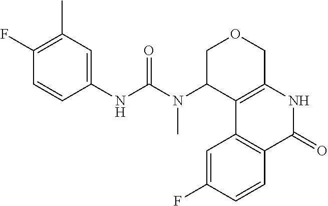 [SMILES: Cc1cc(NC(=O)N(C)C2COCc3[nH]c(=O)c4ccc(F)cc4c32)ccc1F] 3-(4-fluoro-3-methylphenyl)-1-(9-fluoro-6-oxo-1,4,5,6- tetrahydro-2H-pyrano[3,4-c]isoquinolin-1-yl)-1-methylurea | 0.02 |
| 76 | 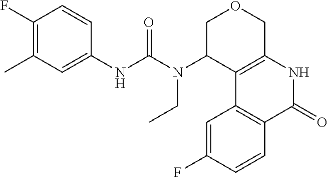 [SMILES: CCN(C(=O)Nc1ccc(F)c(C)c1)C1COCc2[nH]c(=O)c3ccc(F)cc3c21] 1-ethyl-3-(4-fluoro-3-methylphenyl)-1-(9-fluoro-6-oxo- 1,4,5,6-tetrahydro-2H-pyrano[3,4-c]isoquinolin-1-yl)urea | 0.01 |
| 77 | 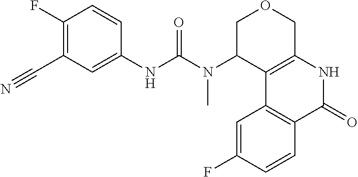 [SMILES: CN(C(=O)Nc1ccc(F)c(C#N)c1)C1COCc2[nH]c(=O)c3ccc(F)cc3c21] 3-(3-cyano-4-fluorophenyl)-1-(9-fluoro-6-oxo-1,4,5,6- tetrahydro-2H-pyrano[3,4-c]isoquinolin-1-yl)-1-methylurea | 0.05 |
| 78 | 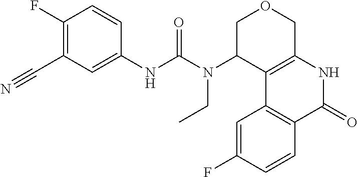 [SMILES: CCN(C(=O)Nc1ccc(F)c(C#N)c1)C1COCc2[nH]c(=O)c3ccc(F)cc3c21] 3-(3-cyano-4-fluorophenyl)-1-ethyl-1-(9-fluoro-6-oxo- 1,4,5,6-tetrahydro-2H-pyrano[3,4-c]isoquinolin-1-yl)urea | 0.04 |
| 79 | 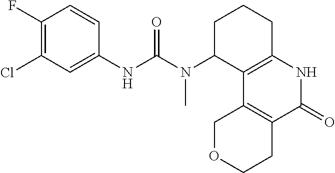 [SMILES: CN(C(=O)Nc1ccc(F)c(Cl)c1)C1CCCc2[nH]c(=O)c3c(c21)COCC3] 3-(3-chloro-4-fluorophenyl)-1-methyl-1-(5-oxo- 3,4,5,6,7,8,9,10- octahydro-1H-pyrano[4,3-c]quinolin- 10-yl)urea; Enantiomer I | 25 |
| 80 | 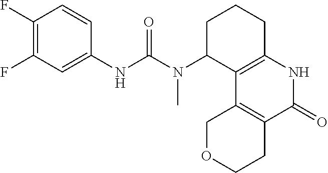 [SMILES: CN(C(=O)Nc1ccc(F)c(F)c1)C1CCCc2[nH]c(=O)c3c(c21)COCC3] 3-(3-chloro-4-fluorophenyl)-1-methyl-1-(5-oxo- 3,4,5,6,7,8,9,10-octahydro-1H-pyrano[4,3-c]quinolin- 10-yl)urea; Enantiomer II | 2.8 |
| 81 | 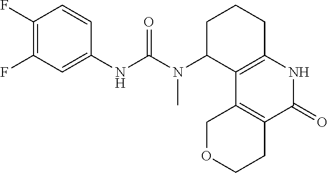 [SMILES: CN(C(=O)Nc1ccc(F)c(F)c1)C1CCCc2[nH]c(=O)c3c(c21)COCC3] 3-(3,4-difluorophenyl)-1-methyl-1-(5-oxo- 3,4,5,6,7,8,9,10-octahydro-1H-pyrano[4,3-c]quinolin- 10-yl)urea; Enantiomer I | 25 |
| 82 | 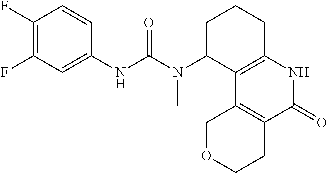 [SMILES: CN(C(=O)Nc1ccc(F)c(F)c1)C1CCCc2[nH]c(=O)c3c(c21)COCC3] 3-(3,4-difluorophenyl)-1-methyl-1-(5-oxo- 3,4,5,6,7,8,9,10-octahydro-1H-pyrano[4,3-c]quinolin- 10-yl)urea; Enantiomer II | 7.3 |
| 83 | 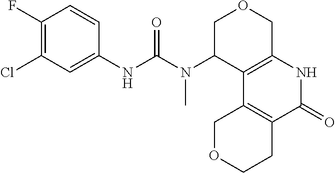 [SMILES: CN(C(=O)Nc1ccc(F)c(Cl)c1)C1COCc2[nH]c(=O)c3c(c21)COCC3] 3-(3-chloro-4-fluorophenyl)-1-methyl-1-(5-oxo- 4,5,6,7,9,10-hexahydro-1H,3H-dipyrano [3,4-b:3′,4′-d]pyridin-10-yl)urea; Enantiomer I | 25 |
| 84 | 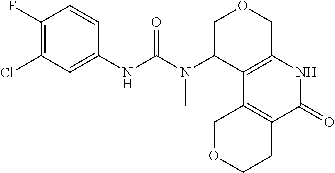 [SMILES: CN(C(=O)Nc1ccc(F)c(Cl)c1)C1COCc2[nH]c(=O)c3c(c21)COCC3] 3-(3-chloro-4-fluorophenyl)-1-methyl-1-(5-oxo- 4,5,6,7,9,10-hexahydro-1H,3H-dipyrano [3,4-b:3',4'-d]pyridin-10-yl)urea; Enantiomer II | 0.83 |
| 85 | 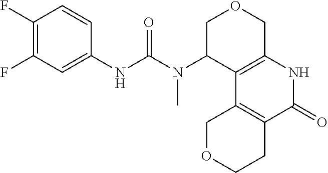 [SMILES: CN(C(=O)Nc1ccc(F)c(F)c1)C1COCc2[nH]c(=O)c3c(c21)COCC3] 3-(3,4-difluorophenyl)-1-methyl-1-(5-oxo-4,5,6,7,9,10- hexahydro-1H,3H-dipyrano[3,4-b:3',4'-d]pyridin- 10-yl)urea; Enantiomer I | 4.5 |
| 86 | 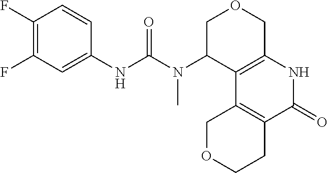 [SMILES: CN(C(=O)Nc1ccc(F)c(F)c1)C1COCc2[nH]c(=O)c3c(c21)COCC3] 3-(3,4-difluorophenyl)-1-methyl-1-(5-oxo-4,5,6,7,9,10- hexahydro-1H,3H-dipyrano[3,4-b 3',4'-d]pyridin- 10-yl)urea; Enantiomer II | 25 |
| 87 | 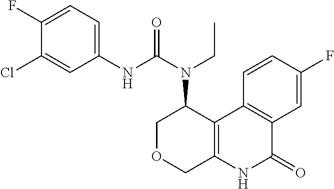 [SMILES: CCN(C(=O)Nc1ccc(F)c(Cl)c1)[C@@H]1COCc2[nH]c(=O)c3cc(F)ccc3c21] (S)-3-(3-chloro-4-fluorophenyl)-1-ethyl-1-(8-fluoro-6- oxo-1,4,5,6- tetrahydro-2H-pyrano[3,4-c]isoquinolin-1- yl)urea; Single enantiomer | 0.01 |
| 88 | 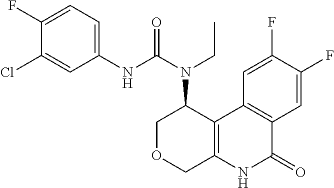 [SMILES: CCN(C(=O)Nc1ccc(F)c(Cl)c1)[C@@H]1COCc2[nH]c(=O)c3cc(F)c(F)cc3c21] (S)-3-(3-chloro-4-fluorophenyl)-1-(8,9-difluoro-6-oxo- 1,4,5,6-tetrahydro-2H-pyrano[3,4-c]isoquinolin-1-yl)-1- ethylurea; Single enantiomer | 0.01 |
| 89 | 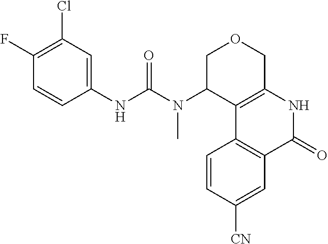 [SMILES: CN(C(=O)Nc1ccc(F)c(Cl)c1)C1COCc2[nH]c(=O)c3cc(C#N)ccc3c21] 3-(3-chloro-4-fluorophenyl)-1-(8-cyano-6-oxo-1,4,5,6- tetrahydro-2H-pyrano[3,4-c]isoquinolin-1-yl)-1- methylurea | 1.1 |
| 90 | 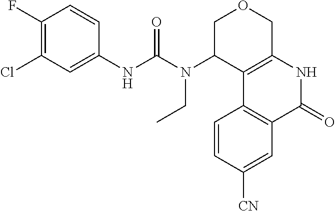 [SMILES: CCN(C(=O)Nc1ccc(F)c(Cl)c1)C1COCc2[nH]c(=O)c3cc(C#N)ccc3c21] 3-(3-chloro-4-fluorophenyl)-1-(8-cyano-6-oxo-1,4,5,6- tetrahydro-2H-pyrano[3,4-c]isoquinolin-1-yl)-1- ethylurea | 0.36 |
| 91 | 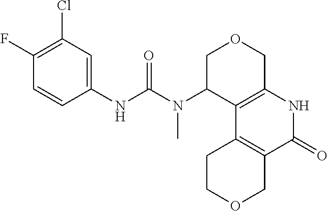 [SMILES: CN(C(=O)Nc1ccc(F)c(Cl)c1)C1COCc2[nH]c(=O)c3c(c21)CCOC3] 3-(3-chloro-4-fluorophenyl)-1-methyl-1-(6-oxo- 1,2,4,5,6,7,9,10-octahydrodipyrano[3,4-b:3',4'-d] pyridin-1-yl)urea; Enantiomer I | 11 |
| 92 | 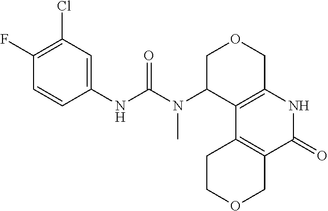 [SMILES: CN(C(=O)Nc1ccc(F)c(Cl)c1)C1COCc2[nH]c(=O)c3c(c21)CCOC3] 3-(3-chloro-4-fluorophenyl)-1-methyl-1-(6-oxo- 1,2,4,5,6,7,9,10-octahydrodipyrano[3,4-b:4',3'-d] pyridin-1-yl)urea; Enantiomer II | 0.15 |
| 93 | 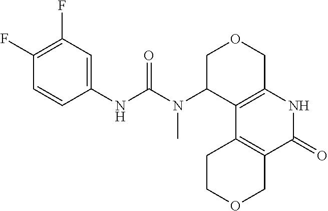 [SMILES: CN(C(=O)Nc1ccc(F)c(F)c1)C1COCc2[nH]c(=O)c3c(c21)CCOC3] 3-(3,4-difluorophenyl)-1-methyl-1-(6-oxo- 1,2,4,5,6,7,9,10-octahydrodipyrano[3,4-b:4',3'-d] pyridin-1-yl)urea; Enantiomer I | 25 |
| 94 | 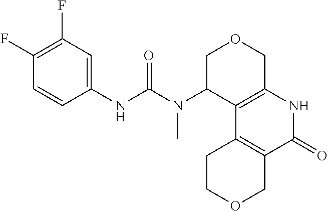 [SMILES: CN(C(=O)Nc1ccc(F)c(F)c1)C1COCc2[nH]c(=O)c3c(c21)CCOC3] 3-(3,4-difluorophenyl)-1-methyl-1-(6-oxo- 1,2,4,5,6,7,9,10-octahydrodipyrano[3,4-b:4',3'-d] pyridin-1-yl)urea; Enantiomer II | 0.74 |
| 95 | 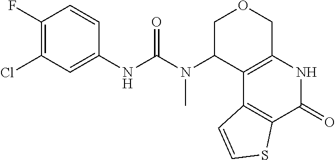 [SMILES: CN(C(=O)Nc1ccc(F)c(Cl)c1)C1COCc2[nH]c(=O)c3sccc3c21] 3-(3-chloro-4-fluorophenyl)-1-methyl-1-{6-oxo-l ,4,5,6- tetrahydro-2H-pyrano[3,4-b]thieno[3,2-d]pyridin-1-yl)urea | 0.03 |
| 96 | 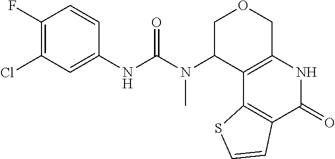 [SMILES: CN(C(=O)Nc1ccc(F)c(Cl)c1)C1COCc2[nH]c(=O)c3ccsc3c21] 3-(3-chloro-4-fluorophenyl)-1-methyl-1-(4-oxo-4,6,8,9- tetrahydro-5H-pyrano[3,4-b]thieno[2,3-d]pyridin-9-yl)urea | 0.08 |
| 97 | 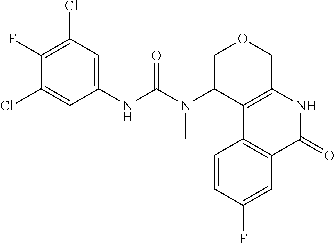 [SMILES: CN(C(=O)Nc1cc(Cl)c(F)c(Cl)c1)C1COCc2[nH]c(=O)c3cc(F)ccc3c21] 3-(3,5-dichloro-4-fluorophenyl)-1-(8-fluoro-6-oxo-1,4,5,6- tetrahydro-2H-pyrano[3,4-c]isoquinolin-1-yl)-1-methylurea; Enantiomer I | 3.3 |
| 98 | 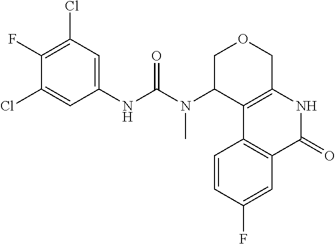 [SMILES: CN(C(=O)Nc1cc(Cl)c(F)c(Cl)c1)C1COCc2[nH]c(=O)c3cc(F)ccc3c21] 3-(3,5-dichloro-4-fluorophenyl)-1-(8-fluoro-6-oxo-1,4,5,6- tetrahydro-2H-pyrano[3,4-c]isoquinolin-1-yl)-1-methylurea; Enantiomer II | 0.03 |
| 99 | 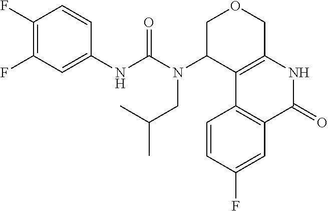 [SMILES: CC(C)CN(C(=O)Nc1ccc(F)c(F)c1)C1COCc2[nH]c(=O)c3cc(F)ccc3c21] 3-(3,4-difluorophenyl)-1-(8-fluoro-6-oxo-1,4,5,6- tetrahydro-2H-pyrano[3,4-c]isoquinolin-1-yl)-1- isobutylurea; Enantiomer I | 0.01 |
| 100 | 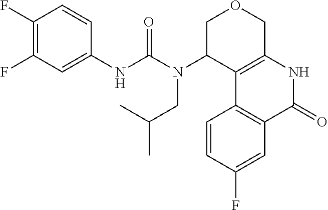 [SMILES: CC(C)CN(C(=O)Nc1ccc(F)c(F)c1)C1COCc2[nH]c(=O)c3cc(F)ccc3c21] 3-(3,4-difluorophenyl)-1-(8-fluoro-6-oxo-1,4,5,6- tetrahydro-2H-pyrano[3,4-c]isoquinolin-1-yl)-1- isobutylurea; Enantiomer II | 0.43 |
| 101 | 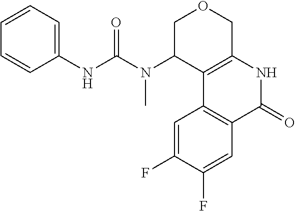 [SMILES: CN(C(=O)Nc1ccccc1)C1COCc2[nH]c(=O)c3cc(F)c(F)cc3c21] 1-(8,9-difluoro-6-oxo-1,4,5,6-tetrahydro-2H-pyrano [3,4-c]isoquinolin-1-yl)-1-methyl-3-phenylurea; Enantiomer I | 4.7 |
| 102 | 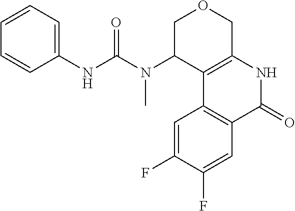 [SMILES: CN(C(=O)Nc1ccccc1)C1COCc2[nH]c(=O)c3cc(F)c(F)cc3c21] 1-(8,9-difluoro-6-oxo-1,4,5,6-tetrahydro-2H- pyrano[3,4-c]isoquinolin-1-yl)-1-methyl-3- phenylurea; Enantiomer II | 0.03 |
| 103 | 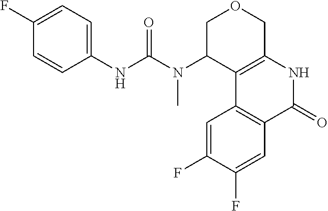 [SMILES: CN(C(=O)Nc1ccc(F)cc1)C1COCc2[nH]c(=O)c3cc(F)c(F)cc3c21] 1-(8,9-difluoro-6-oxo-1,4,5,6-tetrahydro-2H-pyrano [3,4-c]isoquinolin-1-yl)-3-(4-fluorophenyl)-1- methylurea; Enantiomer I | 5.3 |
| 104 | 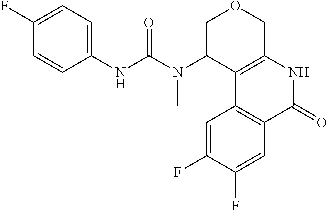 [SMILES: CN(C(=O)Nc1ccc(F)cc1)C1COCc2[nH]c(=O)c3cc(F)c(F)cc3c21] 1-(8,9-difluoro-6-oxo-l ,4,5,6-tctrahydro-2H-pyrano [3,4-c]isoquinolin-1-yl)-3-(4-fluorophenyl)-1- methylurea; Enantiomer II | 0.02 |
| 105 | 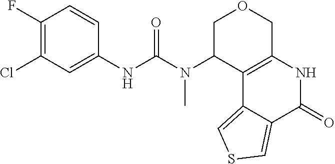 [SMILES: CN(C(=O)Nc1ccc(F)c(Cl)c1)C1COCc2[nH]c(=O)c3cscc3c21] 3-(3-chloro-4-fluorophenyl)-1-methyl-1-(4-oxo-4,5,8,9- tetrahydro-6H-pyrano[3,4-b]thieno[3,4-d]pyridin-9-yl)urea | 0.02 |
| 106 | 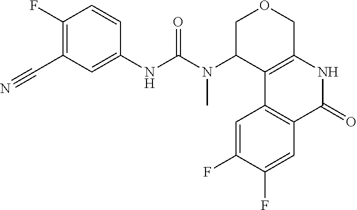 [SMILES: CN(C(=O)Nc1ccc(F)c(C#N)c1)C1COCc2[nH]c(=O)c3cc(F)c(F)cc3c21] 3-(3-cyano-4-fluorophenyl)-1-(8,9-difluoro-6-oxo-1,4,5,6- tetrahydro-2H-pyrano[3,4-c]isoquinolin-1-yl)-1-methylurea | 0.02 |
| 107 | 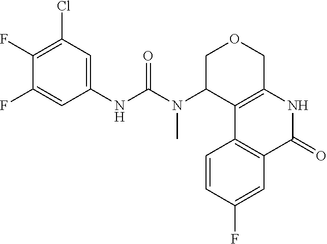 [SMILES: CN(C(=O)Nc1cc(F)c(F)c(Cl)c1)C1COCc2[nH]c(=O)c3cc(F)ccc3c21] 3-(3-chloro-4,5-difluorophenyl)-1-(8-fluoro-6-oxo- 1,4,5,6-tetrahydro-2H-pyrano[3,4-c]isoquinolin-1-yl)-1- methylurea; Enantiomer I | 0.93 |
| 108 | 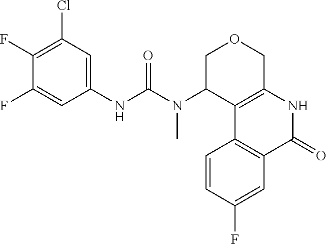 [SMILES: CN(C(=O)Nc1cc(F)c(F)c(Cl)c1)C1COCc2[nH]c(=O)c3cc(F)ccc3c21] 3-(3-chloro-4,5-difluorophenyl)-1-(8-fluoro-6-oxo- 1,4,5,6-tetrahydro-2H-pyrano[3,4-c]isoquinolin-1-yl)-1- methylurea; Enantiomer II | 0.02 |
| 109 | 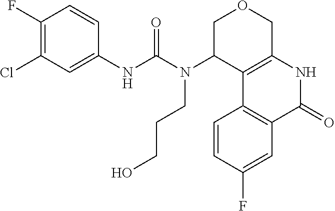 [SMILES: O=C(Nc1ccc(F)c(Cl)c1)N(CCCO)C1COCc2[nH]c(=O)c3cc(F)ccc3c21] 3-(3-chloro-4-fluorophenyl)-1-(8-fluoro-6-oxo-1,4,5,6- tetrahydro-2H-pyrano[3,4-c]isoquinolin-1-yl)-1-(3- hydroxypropyl)urea; Enantiomer I | 0.62 |
| 110 | 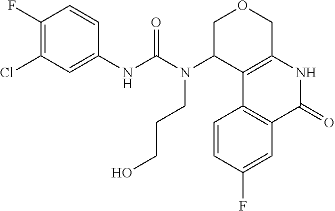 [SMILES: O=C(Nc1ccc(F)c(Cl)c1)N(CCCO)C1COCc2[nH]c(=O)c3cc(F)ccc3c21] 3-(3-chloro-4-fluorophenyl)-1-(8-fluoro-6-oxo- 1,4,5,6-tetrahydro-2H-pyrano[3,4-c]isoquinolin-1-yl)- 1-(3-hydroxypropyl)urea; Enantiomer II | 0.02 |
| 111 | 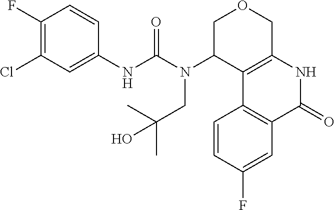 [SMILES: CC(C)(O)CN(C(=O)Nc1ccc(F)c(Cl)c1)C1COCc2[nH]c(=O)c3cc(F)ccc3c21] 3-(3-chloro-4-fluorophenyl)-1-(8-fluoro-6-oxo-1,4,5,6- tetrahydro-2H-pyrano[3,4-c]isoquinolin-1-yl)-1-(2- hydroxy-2-methylpropyl)urea; Enantiomer I | 0.20 |
| 112 | 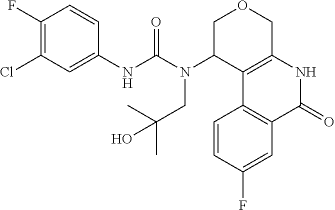 [SMILES: CC(C)(O)CN(C(=O)Nc1ccc(F)c(Cl)c1)C1COCc2[nH]c(=O)c3cc(F)ccc3c21] 3-(3-chloro-4-fluorophenyl)-1-(8-fluoro-6-oxo-1,4,5,6- tetrahydro-2H-pyrano[3,4-c]isoquinolin-1-yl)-1-(2- hydroxy-2-methylpropyl)urea; Enantiomer II | 1.9 |
| 113 | 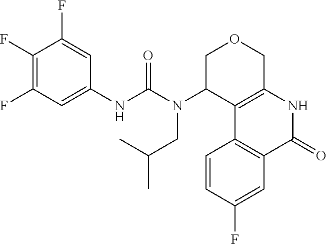 [SMILES: CC(C)CN(C(=O)Nc1cc(F)c(F)c(F)c1)C1COCc2[nH]c(=O)c3cc(F)ccc3c21] 1-(8-fluoro-6-oxo-1,4,5,6-tetrahydro-2H-pyrano[3,4-c] isoquinolin-l-yl)-1-isobutyl-3-(3,4,5- trifluorophenyl)urea; Enantiomer I | 0.02 |
| 114 | 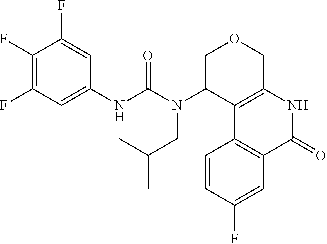 [SMILES: CC(C)CN(C(=O)Nc1cc(F)c(F)c(F)c1)C1COCc2[nH]c(=O)c3cc(F)ccc3c21] 1-(8-fluoro-6-oxo-1,4,5,6-tetrahydro-2H-pyrano[3,4-c] isoquinolin-l-yl)-1-isobutyl-3-(3,4,5- trifluorophenyl)urea; Enantiomer II | 0.41 |
| 115 | 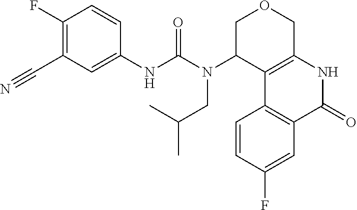 [SMILES: CC(C)CN(C(=O)Nc1ccc(F)c(C#N)c1)C1COCc2[nH]c(=O)c3cc(F)ccc3c21] 3-(3-cyano-4-fluorophenyl)-1-(8-fluoro-6-oxo-1,4,5,6- tetrahydro-2H-pyrano[3,4-c]isoquinolin-1-yl)-1- isobutylurea; Enantiomer I | 0.01 |
| 116 | 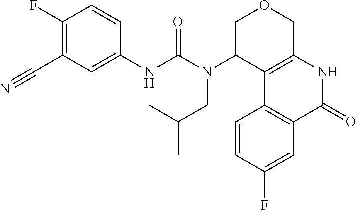 [SMILES: CC(C)CN(C(=O)Nc1ccc(F)c(C#N)c1)C1COCc2[nH]c(=O)c3cc(F)ccc3c21] 3-(3-cyano-4-fluorophenyl)-1-(8-fluoro-6-oxo-l ,4,5,6- tetrahydro- 2H-pyrano[3,4-c]isoquinolin-1-yl)-1- isobutylurea; Enantiomer II | 0.23 |
| 117 | 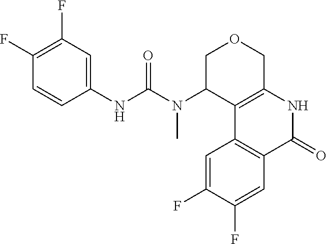 [SMILES: CN(C(=O)Nc1ccc(F)c(F)c1)C1COCc2[nH]c(=O)c3cc(F)c(F)cc3c21] 1-(8,9-difluoro-6-oxo-l ,4,5,6-tetrahydro-2H- pyrano[3,4-c]isoquinolin-1-yl)-3-(3,4-difluorophenyl)-1- methylurea; Enantiomer I | 2.7 |
| 118 | 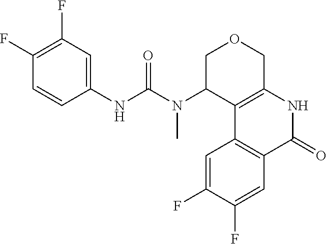 [SMILES: CN(C(=O)Nc1ccc(F)c(F)c1)C1COCc2[nH]c(=O)c3cc(F)c(F)cc3c21] 1-(8,9-difluoro-6-oxo-1,4,5,6-tetrahydro-2H-pyrano [3,4-c]isoquinolin-1-yl)-3-(3,4-difluorophenyl)-1- methylurea; Enantiomer II | 0.01 |
| 119 | 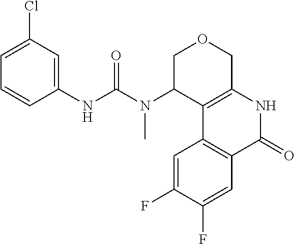 [SMILES: CN(C(=O)Nc1cccc(Cl)c1)C1COCc2[nH]c(=O)c3cc(F)c(F)cc3c21] 3-(3-chlorophenyl)-1-(8,9-difluoro-6-oxo-1,4,5,6- tetrahydro-2H-pyrano[3,4-c]isoquinolin-1-yl)-1- methylurea; Enantiomer I | 1.8 |
| 120 | 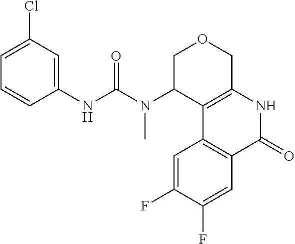 [SMILES: CN(C(=O)Nc1cccc(Cl)c1)C1COCc2[nH]c(=O)c3cc(F)c(F)cc3c21] 3-(3-chlorophenyl)-1-(8,9-difluoro-6-oxo-1,4,5,6- tetrahydro-2H-pyrano[3,4-c]isoquinolin-1-yl)-1- methylurea; Enantiomer II | 0.01 |
| 121 | 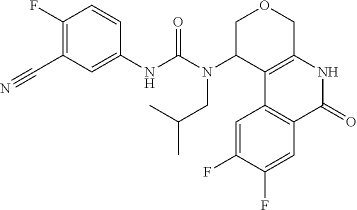 [SMILES: CC(C)CN(C(=O)Nc1ccc(F)c(C#N)c1)C1COCc2[nH]c(=O)c3cc(F)c(F)cc3c21] 3-(3-cyano-4-fluorophenyl)-1-(8,9-difluoro-6-oxo-1,4,5,6- tetrahydro-2H-pyrano[3,4-c]isoquinolin-1-yl)-1-isobutylurea; Enantiomer I | 0.01 |
| 122 | 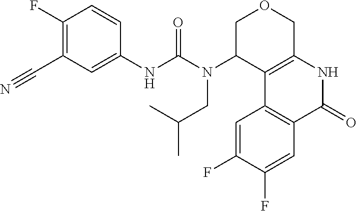 [SMILES: CC(C)CN(C(=O)Nc1ccc(F)c(C#N)c1)C1COCc2[nH]c(=O)c3cc(F)c(F)cc3c21] 3-(3-cyano-4-fluorophenyl)-1-(8,9-difluoro-6-oxo-1,4,5,6- tetrahydro-2H-pyrano[3,4-c]isoquinolin-1-yl)-1-isobutylurea; Enantiomer II | 0.12 |
| 123 | 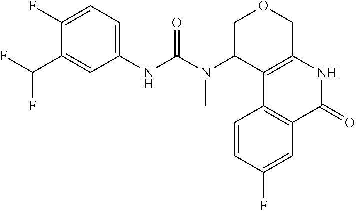 [SMILES: CN(C(=O)Nc1ccc(F)c(C(F)F)c1)C1COCc2[nH]c(=O)c3cc(F)ccc3c21] 3-(3-(difluoromethyl)-4-fluoroplienyl)-1-(8-fluoro-6-oxo-1,4,5,6- tetrahydro-2H-pyrano[3,4-c]isoquinolin-1-yl)-1-methylurea; Enantiomer I | 0.56 |
| 124 | 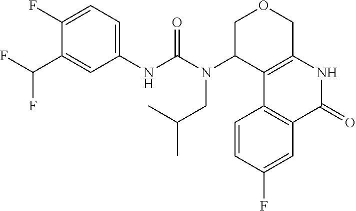 [SMILES: CC(C)CN(C(=O)Nc1ccc(F)c(C(F)F)c1)C1COCc2[nH]c(=O)c3cc(F)ccc3c21] 3-(3-(difluoromethyl)-4-fluorophenyl)-1-(8-fluoro-6-oxo- 1,4,5,6-tetrahydro-2H-pyrano[3,4-c]isoquinolin-1-yl)- 1-methylurea; Enantiomer II | 0.01 |
| 125 | 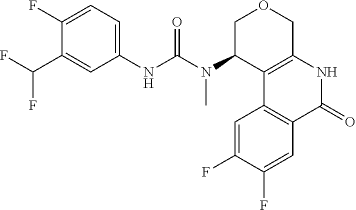 [SMILES: CN(C(=O)Nc1ccc(F)c(C(F)F)c1)[C@H]1COCc2[nH]c(=O)c3cc(F)c(F)cc3c21] (R)-1-(8,9-difluoro-6-oxo-1,4,5,6-tetrahydro-2H- pyrano[3,4-c]isoquinolin-1-yl)-3-(3-(difluoromethyl)-4- fluorophenyl)-1-methylurea; Enantiomer I | 0.49 |
| 126 | 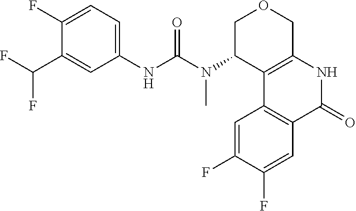 [SMILES: CN(C(=O)Nc1ccc(F)c(C(F)F)c1)[C@@H]1COCc2[nH]c(=O)c3cc(F)c(F)cc3c21] (S)-1-(8,9-difluoro-6-oxo-1,4,5,6-tetrahydro-2H-pyrano[3,4-c] isoquinolin-1-yl)-3-(3-(difluoromethyl)-4-fluorophenyl)- 1-methylurea; Enantiomer II | 0.01 |
| 131 | 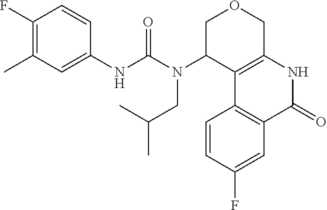 [SMILES: Cc1cc(NC(=O)N(CC(C)C)C2COCc3[nH]c(=O)c4cc(F)ccc4c32)ccc1F] 3-(4-fluoro-3-methylphenyl)-1-(8-fluoro-6-oxo-1,4,5,6- tetrahydro-2H-pyrano[3,4-c]isoquinolin-1-yl)- 1-isobutylurea; Enantiomer I | 0.01 |
| 132 | 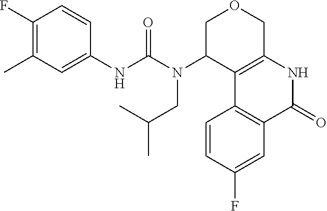 [SMILES: Cc1cc(NC(=O)N(CC(C)C)C2COCc3[nH]c(=O)c4cc(F)ccc4c32)ccc1F] 3-(4-fluoro-3-methylphenyl)-1-(8-fluoro-6-oxo-1,4,5,6- tetrahydro-2H-pyrano[3,4-c]isoquinolin-1-yl)- 1-isobutylurea; Enantiomer II | 0.15 |
| 133 | 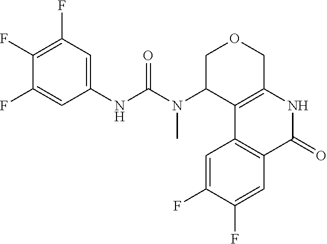 [SMILES: CN(C(=O)Nc1cc(F)c(F)c(F)c1)C1COCc2[nH]c(=O)c3cc(F)c(F)cc3c21] 1-(8,9-difluoro-6-oxo-1,4,5,6-tetrahydro-2H- pyrano[3,4-c]isoquinolin-1-yl)-1-methyl-3-(3,4,5- trifluorophenyl)urea; Enantiomer I | 0.02 |
| 134 | 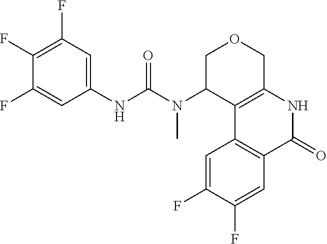 [SMILES: CN(C(=O)Nc1cc(F)c(F)c(F)c1)C1COCc2[nH]c(=O)c3cc(F)c(F)cc3c21] 1-(8,9-difluoro-6-oxo-1,4,5,6-tetrahydro-2H-pyrano[3,4-c] isoquinolin-1-yl)-1-methyl-3-(3,4,5-trifluorophenyl)urea; Enantiomer II | 0.99 |
| 135 | 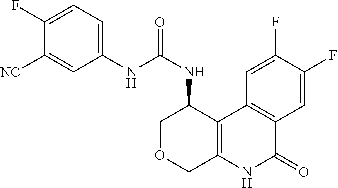 [SMILES: N#Cc1cc(NC(=O)N[C@@H]2COCc3[nH]c(=O)c4cc(F)c(F)cc4c32)ccc1F] (S)-1-(3-cyano-4-fluorophenyl)-3-(8,9-difluoro-6-oxo-1,4,5,6- tetrahydro-2H-pyrano[3,4-c]isoquinolin-1-yl)urea; Single enantiomer | 0.05 |
| 136 | 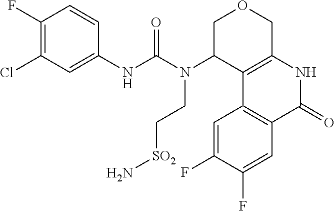 [SMILES: NS(=O)(=O)CCN(C(=O)Nc1ccc(F)c(Cl)c1)C1COCc2[nH]c(=O)c3cc(F)c(F)cc3c21] 2-(3-(3-chloro-4-fluorophenyl)-1-(8,9-difluoro-6-oxo- 1,4,5,6-tetrahydro-2H-pyrano[3,4-c]isoquinolin- 1-yl)ureido)ethane-1-sulfonamide; Enantiomer I | 2.9 |
| 137 | 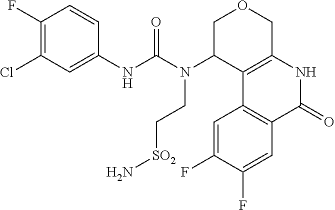 [SMILES: NS(=O)(=O)CCN(C(=O)Nc1ccc(F)c(Cl)c1)C1COCc2[nH]c(=O)c3cc(F)c(F)cc3c21] 2-(3-(3-chloro-4-fluorophenyl)-1-(8,9-difluoro-6-oxo- 1,4,5,6-tetrahydro-2H-pyrano[3,4-c]isoquinolin- 1-yl)ureido)ethane-1-sulfonamide; Enantiomer II | 0.01 |
| 138 | 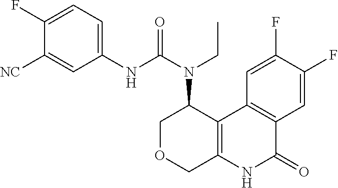 [SMILES: CCN(C(=O)Nc1ccc(F)c(C#N)c1)[C@@H]1COCc2[nH]c(=O)c3cc(F)c(F)cc3c21] (S)-3-(3-cyano-4-fluorophenyl)-1-(8,9-difluoro-6-oxo- 1,4,5,6-tetrahydro-2H-pyrano[3,4-c]isoquinolin- 1-yl)-1-ethylurea; Single enantiomer | 0.01 |
| 139 | 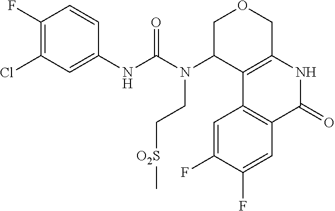 [SMILES: CS(=O)(=O)CCN(C(=O)Nc1ccc(F)c(Cl)c1)C1COCc2[nH]c(=O)c3cc(F)c(F)cc3c21] 3-(3-chloro-4-fluorophenyl)-1-(8,9-difluoro-6-oxo- 1,4,5,6-tetrahydro-2H-pyrano[3,4-c]isoquinolin- 1-yl)-1-(2-(methylsulfonyl)elhyl)urea; Enantiomer I | 0.60 |
| 140 | 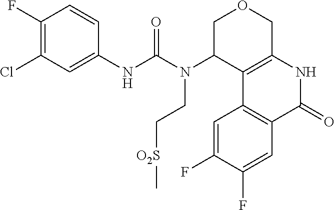 [SMILES: CS(=O)(=O)CCN(C(=O)Nc1ccc(F)c(Cl)c1)C1COCc2[nH]c(=O)c3cc(F)c(F)cc3c21] 3-(3-chloro-4-fluorophenyl)-1-(8,9-difluoro-6-oxo-1,4,5,6- tetrahydro-2H-pyrano[3,4-c]isoquinolin-1-yl)-1-(2- (methylsulfonyl)elhyl)urea; Enantiomer II | 0.01 |
| 141 | 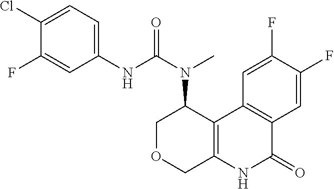 [SMILES: CN(C(=O)Nc1ccc(Cl)c(F)c1)[C@@H]1COCc2[nH]c(=O)c3cc(F)c(F)cc3c21] (S)-3-(4-chloro-3-fluorophenyl)-1-(8,9-difluoro-6-oxo- 1,4,5,6-tetrahydro-2H-pyrano[3,4-c]isoquinolin-1-yl)-1- methylurea | 0.02 |
| 142 | 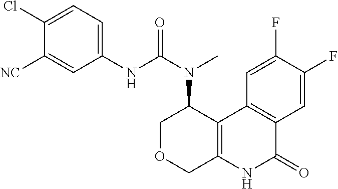 [SMILES: CN(C(=O)Nc1ccc(Cl)c(C#N)c1)[C@@H]1COCc2[nH]c(=O)c3cc(F)c(F)cc3c21] (S)-3-(4-chloro-3-cyanophenyl)-1-(8,9-difluoro-6-oxo- 1,4,5,6-tetrahydro-2H-pyrano[3,4-c]isoquinolin-1-yl)-1- methylurea | 0.03 |
| 143 | 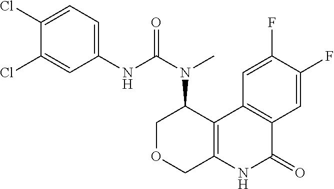 [SMILES: CN(C(=O)Nc1ccc(Cl)c(Cl)c1)[C@@H]1COCc2[nH]c(=O)c3cc(F)c(F)cc3c21] (S)-3-(3,4-dichlorophenyl)-1-(8,9-difluoro-6-oxo-1,4,5,6- tetrahydro-2H-pyrano[3,4-c]isoquinolin-1-yl)-1-methylurea | 0.02 |
| 144 | 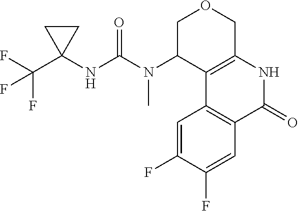 [SMILES: CN(C(=O)NC1(C(F)(F)F)CC1)C1COCc2[nH]c(=O)c3cc(F)c(F)cc3c21] 1-(8,9-difluoro-6-oxo-1,2,3,4,5,6- hexahydrophenanthridin-1-yl)-1-methyl-3-(1- (trifluoromethyl)cyclopropyl)urea | 20 |
| 145 | 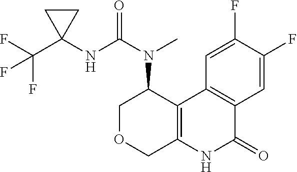 [SMILES: CN(C(=O)NC1(C(F)(F)F)CC1)[C@@H]1COCc2[nH]c(=O)c3cc(F)c(F)cc3c21] (S)-1-(8,9-difluoro-6-oxo-1,4,5,6-tetrahydro-2H- pyrano[3,4-c]isoquinolin-1-yl)-1-methyl-3-(1- (trifluoromethyl)cyclopropyl)urea | 9.0 |
| 146 | 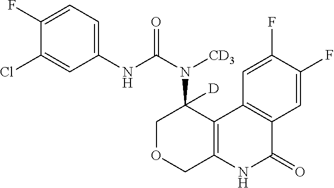 [SMILES: [2H]C([2H])([2H])N(C(=O)Nc1ccc(F)c(Cl)c1)[C@]1([2H])COCc2[nH]c(=O)c3cc(F)c(F)cc3c21] (S)-3-(3-Chloro-4-fluorophenyl)-1-(8,9-difluoro-6-oxo- 1,4,5,6-tetrahydro-2H-pyrano[3,4-c]isoquinolin-1-yl- 1-d)-1-(methyl-d3)urea | 0.01 |
| 147 | 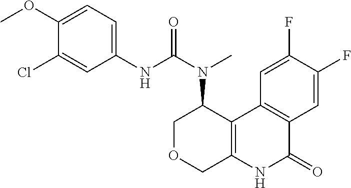 [SMILES: COc1ccc(NC(=O)N(C)[C@@H]2COCc3[nH]c(=O)c4cc(F)c(F)cc4c32)cc1Cl] (S)-3-(3-Chloro-4-methoxyphenyl)-1-(8,9-difluoro-6-oxo- 1,4,5,6-tetrahydro-2H-pyrano[3,4-c]isoquinolin- 1-yl)-1-methylurea | 0.14 |
| 148 | 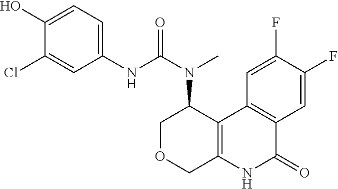 [SMILES: CN(C(=O)Nc1ccc(O)c(Cl)c1)[C@@H]1COCc2[nH]c(=O)c3cc(F)c(F)cc3c21] (S)-3-(3-Chloro-4-hydroxyphenyl)-1-(8,9-difIuoro-6-oxo- 1,4,5,6-tetrahydro-2H-pyrano[3,4-c]isoquinolin-1-yl)- 1-methylurea | 0.76 |
| 149 | 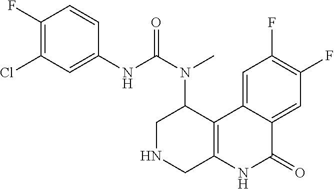 [SMILES: CN(C(=O)Nc1ccc(F)c(Cl)c1)C1CNCc2[nH]c(=O)c3cc(F)c(F)cc3c21] 3-(3-Chloro-4-fluorophenyl)-1-(8,9-difluoro-6-oxo- 1,2,3,4,5,6-hexahydrobenzo[c][1,7]naphthyridin-1-yl)-1- methylurea; Enantiomer I | 0.25 |
| 150 | 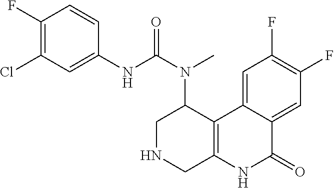 [SMILES: CN(C(=O)Nc1ccc(F)c(Cl)c1)C1CNCc2[nH]c(=O)c3cc(F)c(F)cc3c21] 3-(3-Chloro-4-fluorophenyl)-1-(8,9-difluoro-6-oxo- 1,2,3,4,5,6-hexahydrobenzo[c][1,7]naphthyridin-1-yl)-1- methylurea; Enantiomer II | 0.01 |
| 151 | 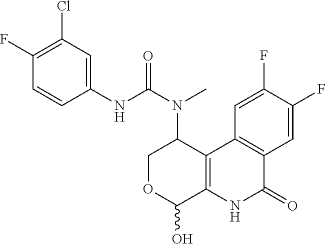 [SMILES: CN(C(=O)Nc1ccc(F)c(Cl)c1)C1COC(O)c2[nH]c(=O)c3cc(F)c(F)cc3c21] 3-(3-Chloro-4-fluorophenyl)-1-(8,9-difluoro-4-hydroxy- 6-oxo-1,4,5,6-tetrahydro-2H-pyrano[3,4-c]isoquinolin-1- yl)-1-methylurea; Mixture of stereoisomers | 0.02 |
| 152 | 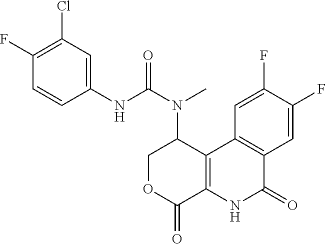 [SMILES: CN(C(=O)Nc1ccc(F)c(Cl)c1)C1COC(=O)c2[nH]c(=O)c3cc(F)c(F)cc3c21] 3-(3-chloro-4-Fluorophenyl)-1-(8,9-difluoro-4,6-dioxo- 1,4,5,6-tetrahydro-2H-pyrano[3,4-c]isoquinolin-1-yl)-1- methylurea | 0.02 |
| 153 | 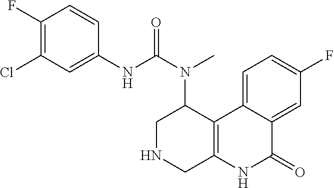 [SMILES: CN(C(=O)Nc1ccc(F)c(Cl)c1)C1CNCc2[nH]c(=O)c3cc(F)ccc3c21] 3-(3-Chloro-4-fluorophenyl)-]-(8-fluoro-6-oxo-1,2,3,4,5,6- hexahydrobenzo[c][1,7]naphthyridin-1-yl)-1-methylurea; Enantiomer I | 0.73 |
| 154 | 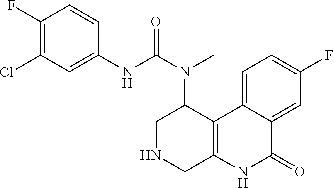 [SMILES: CN(C(=O)Nc1ccc(F)c(Cl)c1)C1CNCc2[nH]c(=O)c3cc(F)ccc3c21] 3-(3-Chloro-4-fluorophenyl)-1-(8-fluoro-6-oxo-1,2,3,4,5,6- hexahydrobenzo[c][1,7]naphthyridin-1-yl)-1-methylurea; Enantiomer II | 0.03 |
| 155 | 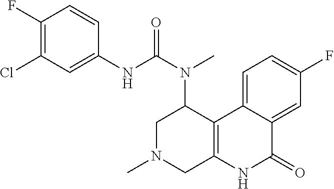 [SMILES: CN1Cc2[nH]c(=O)c3cc(F)ccc3c2C(N(C)C(=O)Nc2ccc(F)c(Cl)c2)C1] 3-(3-Chloro-4-fluorophenyI)-1-(8-fluoro-3-methyl-6-oxo- 1,2,3,4,5,6-hexahydrobenzo[c][1,7]naphthyridin-1-yl)-1- methylurea; Enantiomer I | 9.2 |
| 156 | 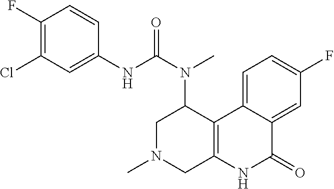 [SMILES: CN1Cc2[nH]c(=O)c3cc(F)ccc3c2C(N(C)C(=O)Nc2ccc(F)c(Cl)c2)C1] 3-(3-Chloro-4-fluorophenyl)-1-(8-fluoro-3-methyl-6-oxo- 1,2,3,4,5,6-hexahydrobenzo[c][1,7]naphthyridin-1-yl)-1- methylurea; Enantiomer II | 0.42 |
| 157 | 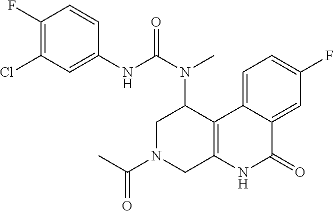 [SMILES: CC(=O)N1Cc2[nH]c(=O)c3cc(F)ccc3c2C(N(C)C(=O)Nc2ccc(F)c(Cl)c2)C1] 1-(3-Acetyl-8-fluoro-6-oxo-l,2,3,4,5,6- hexahydrobenzo[c][1,7]naphthyridin-1-yl)-3-(3-chloro- 4-fluorophenyl)-1-methylurea; Enantiomer I | 1.2 |
| 158 | 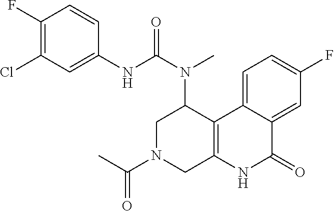 [SMILES: CC(=O)N1Cc2[nH]c(=O)c3cc(F)ccc3c2C(N(C)C(=O)Nc2ccc(F)c(Cl)c2)C1] 1-(3-Acetyl-8-fluoro-6-oxo-1,2,3,4,5,6- hexahydrobenzo[c][1,7]naphthyridin-1-yl)-3-(3- chloro-4-fluorophenyl)-1-methylurea; Enantiomer II | 2.3 |
| 159 | 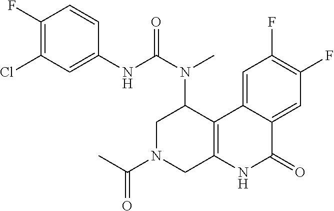 [SMILES: CC(=O)N1Cc2[nH]c(=O)c3cc(F)c(F)cc3c2C(N(C)C(=O)Nc2ccc(F)c(Cl)c2)C1] 1-(3-Acetyl-8,9-difluoro-6-oxo-1,2,3,4,5,6- hexahydrobenzo[c][1,7]naphthyridin-1-yl)-3-(3-chloro-4- fluorophenyl)-1-methylurea; Enantiomer I | 12 |
| 160 | 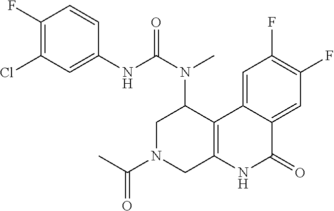 [SMILES: CC(=O)N1Cc2[nH]c(=O)c3cc(F)c(F)cc3c2C(N(C)C(=O)Nc2ccc(F)c(Cl)c2)C1] 1-(3-Acetyl-8,9-difluoro-6-oxo-1,2,3,4,5,6- hexahydrobenzo[c][1,7]naphthyridin-1-yl)-3-(3-chloro- 4-fluorophenyl)-1-methylurea; Enantiomer II | 0.82 |
| 161 | 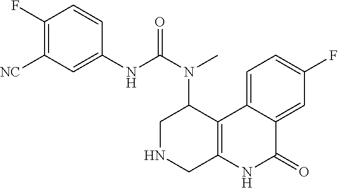 [SMILES: CN(C(=O)Nc1ccc(F)c(C#N)c1)C1CNCc2[nH]c(=O)c3cc(F)ccc3c21] 3-(3-Cyano-4-fluorophenyl)-1-(8-fluoro-6-oxo-1,2,3,4,5,6- hexahydrobenzo[c][1,7]naphthyridin-1-yl)-1-methylurea; Enantiomer I | 17 |
| 162 | 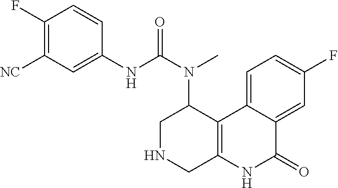 [SMILES: CN(C(=O)Nc1ccc(F)c(C#N)c1)C1CNCc2[nH]c(=O)c3cc(F)ccc3c21] 3-(3-Cyano-4-fluorophenyl)-1-(8-fluoro-6-oxo-1,2,3,4,5,6- hexahydrobenzo[c][1,7]naphthyridin-1-yl)-1-methylurea; Enantiomer II | 0.17 |
| 163 | 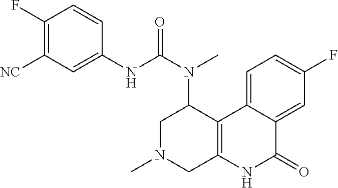 [SMILES: CN1Cc2[nH]c(=O)c3cc(F)ccc3c2C(N(C)C(=O)Nc2ccc(F)c(C#N)c2)C1] 3-(3-Cyano-4-fluorophenyl)-1-(8-fluoro-3-methyl-6-oxo- 1,2,3,4,5,6-hexahydrobenzo[c][1,7]naphthyridin-1- yl)-1-methylurea; Enantiomer I | 23 |
| 164 | 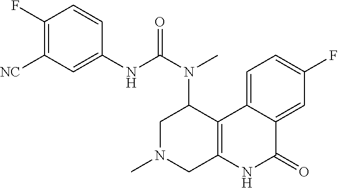 [SMILES: CN1Cc2[nH]c(=O)c3cc(F)ccc3c2C(N(C)C(=O)Nc2ccc(F)c(C#N)c2)C1] 3-(3-Cyano-4-fluorophenyl)-1-(8-fluoro-3-methyl-6-oxo- 1,2,3,4,5,6-hexahydrobenzo[c][1,7]naphthyridin-1- yl)-1-methylurea; Enantiomer II | 1.3 |
| 165 | 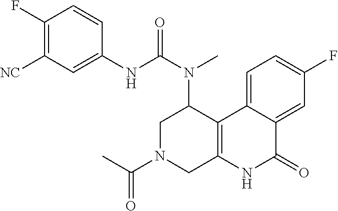 [SMILES: CC(=O)N1Cc2[nH]c(=O)c3cc(F)ccc3c2C(N(C)C(=O)Nc2ccc(F)c(C#N)c2)C1] 1-(3-Acetyl-8-fluoro-6-oxo-1,2,3,4,5,6- hexahydrobenzo[c][1,7]naphthyridin-1-yl)-3-(3-cyano-4- fluorophenyl)-1-methylurea; Enantiomer I | 25 |
| 166 | 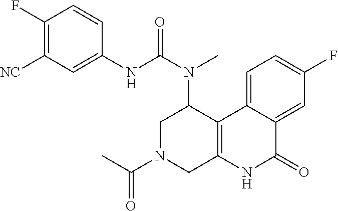 [SMILES: CC(=O)N1Cc2[nH]c(=O)c3cc(F)ccc3c2C(N(C)C(=O)Nc2ccc(F)c(C#N)c2)C1] 1-(3-Acetyl-8-fluoro-6-oxo-1,2,3,4,5,6- hexahydrobenzo[c][1,7]naphthyridin-1-yl)-3-(3-cyano-4- fluorophenyl)-1-methylurea, Enantiomer II | 11 |
| 167 | 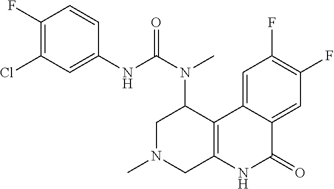 [SMILES: CN1Cc2[nH]c(=O)c3cc(F)c(F)cc3c2C(N(C)C(=O)Nc2ccc(F)c(Cl)c2)C1] 3-(3-Chloro-4-fluorophenyl)-1-(8,9-difluoro-3-methyl-6- oxo-1,2,3,4,5,6-hexahydrobenzo[c][1,7]naphthyridin-1- yl)-1-methylurea; Enantiomer I | 7.6 |
| 168 | 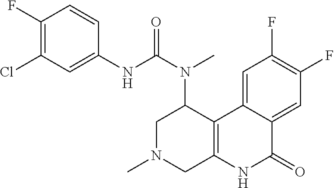 [SMILES: CN1Cc2[nH]c(=O)c3cc(F)c(F)cc3c2C(N(C)C(=O)Nc2ccc(F)c(Cl)c2)C1] 3-(3-Chloro-4-fluorophenyl)-1-(8,9-difluoro-3-methyl-6- oxo-1,2,3,4,5,6-hexahydrobenzo[c][1,7]naphthyridin-1- yl)-1-methylurea; Enantiomer II | 0.18 |
| 169 | 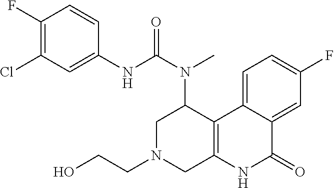 [SMILES: CN(C(=O)Nc1ccc(F)c(Cl)c1)C1CN(CCO)Cc2[nH]c(=O)c3cc(F)ccc3c21] 3-(3-Chloro-4-fluorophenyl)-1-(8-fluoro-3-(2- hydroxyethyl)-6-oxo-1,2,3,4,5,6-hexahydrobenzo[c][1,7] naphthyridin-1-yl)-1-methylurea; Enantiomer I | 6.6 |
| 170 | 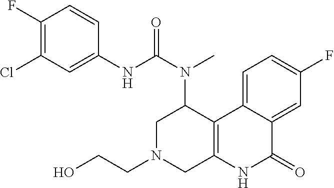 [SMILES: CN(C(=O)Nc1ccc(F)c(Cl)c1)C1CN(CCO)Cc2[nH]c(=O)c3cc(F)ccc3c21] 3-(3-Chloro-4-fluorophenyl)-1-(8-fluoro-3-(2- hydroxyethyl)-6-oxo-1,2,3,4,5,6-hexahydrobenzo[c][1,7] naphthyridin-1-yl)-1-methylurea; Enantiomer II | 3.1 |
| 171 | 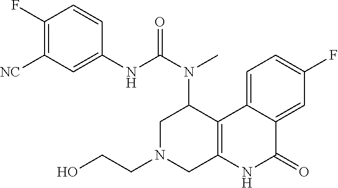 [SMILES: CN(C(=O)Nc1ccc(F)c(C#N)c1)C1CN(CCO)Cc2[nH]c(=O)c3cc(F)ccc3c21] 3-(3-Cyano-4-fluorophenyl)-1-(8-fluoro-3-(2- hydroxyethyl)-6-oxo-1,2,3,4,5,6-hexahydrobenzo[c][1,7] naphthyridin-1-yl)-1-methylurea; Enantiomer I | 23 |
| 172 | 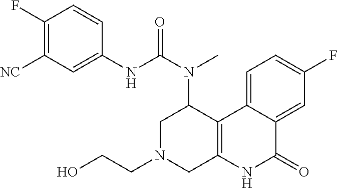 [SMILES: CN(C(=O)Nc1ccc(F)c(C#N)c1)C1CN(CCO)Cc2[nH]c(=O)c3cc(F)ccc3c21] 3-(3-Cyano-4-fluorophenyl)-1-(8-fluoro-3-(2- hydroxyethyl)-6-oxo-1,2,3,4,5,6-hexahydrobenzo[c][1,7] naphthyridin-1-yl)-1-methylurea; Enantiomer II | 10 |
| 173 | 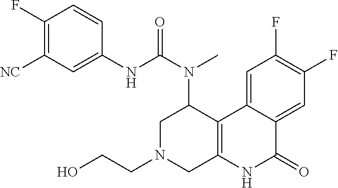 [SMILES: CN(C(=O)Nc1ccc(F)c(C#N)c1)C1CN(CCO)Cc2[nH]c(=O)c3cc(F)c(F)cc3c21] 3-(3-Cyano-4-fluorophenyl)-1-(8,9-difluoro-3-methyl-6- oxo-1,2,3,4,5,6-hexahydrobenzo[c][1,7]naphthyridin-1- yl)-1-methylurea; Enantiomer I | 13 |
| 174 | 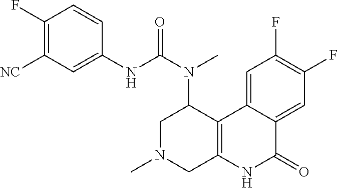 [SMILES: CN1Cc2[nH]c(=O)c3cc(F)c(F)cc3c2C(N(C)C(=O)Nc2ccc(F)c(C#N)c2)C1] 3-(3-Cyano-4-fluorophenyl)-1-(8,9-difluoro-3-methyl-6- oxo-1,2,3,4,5,6-hexahydrobenzo[c][1,7]naphthyridin-1- yl)-1-methylurea; Enantiomer II | 0.31 |
| 175 | 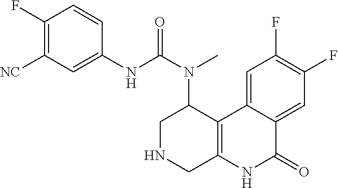 [SMILES: CN(C(=O)Nc1ccc(F)c(C#N)c1)C1CNCc2[nH]c(=O)c3cc(F)c(F)cc3c21] 3-(3-Cyano-4-fluorophenyl)-1-(8,9-difluoro-6-oxo- 1,2,3,4,5,6-hexahydrobenzo[c][1,7]naphthyridin-1-yl)-1- methylurea; Enantiomer I | 1.5 |
| 176 | 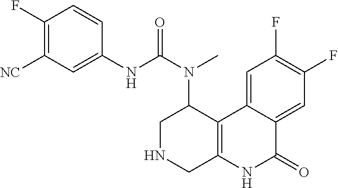 [SMILES: CN(C(=O)Nc1ccc(F)c(C#N)c1)C1CNCc2[nH]c(=O)c3cc(F)c(F)cc3c21] 3-(3-Cyano-4-fluorophenyl)-1-(8,9-dmuoro-6-oxo- 1,2,3,4,5,6-hexahydrobenzo[c][1,7]naphthyridin-1-yl)-1- methylurea; Enantiomer II | 0.05 |
| 177 | 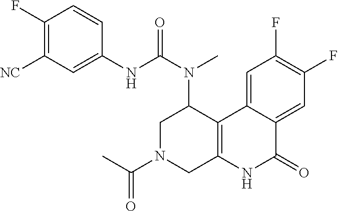 [SMILES: CC(=O)N1Cc2[nH]c(=O)c3cc(F)c(F)cc3c2C(N(C)C(=O)Nc2ccc(F)c(C#N)c2)C1] 1-(3-Acetyl-8,9-difluoro-6-oxo-1,2,3,4,5,6- hexahydrobenzo[c][1,7]naphthyridin-1-yl)-3-(3-cyano-4- fluorophenyl)-1-methylurea; Enantiomer I | 25 |
| 178 | 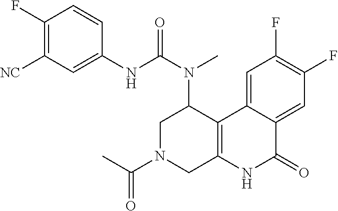 [SMILES: CC(=O)N1Cc2[nH]c(=O)c3cc(F)c(F)cc3c2C(N(C)C(=O)Nc2ccc(F)c(C#N)c2)C1] 1-(3-Acetyl-8,9-difluoro-6-oxo-1,2,3,4,5,6- hexahydrobenzo[c][1,7]naphthyridin-1-yl)-3-(3-cyano-4- fluorophenyl)-1-methylurea; Enantiomer II | 11 |
| 179 | 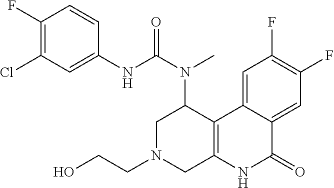 [SMILES: CN(C(=O)Nc1ccc(F)c(Cl)c1)C1CN(CCO)Cc2[nH]c(=O)c3cc(F)c(F)cc3c21] 3-(3-Chloro-4-fluorophenyl)-1-(8,9-difluoro-3-(2- hydroxyethyl)-6-oxo-1,2,3,4,5,6-hexahydrobenzo[c][1,7] naphthyridin-1-yl)-1-methylurea; Enantiomer I | 7.3 |
| 180 | 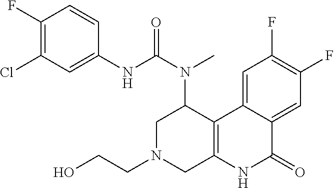 [SMILES: CN(C(=O)Nc1ccc(F)c(Cl)c1)C1CN(CCO)Cc2[nH]c(=O)c3cc(F)c(F)cc3c21] 3-(3-Chloro-4-fluorophenyl)-1-(8,9-difluoro-3-(2- hydroxyethyl)-6-oxo-1,2,3,4,5,6-hexahydrobenzo[c][1,7] naphthyridin-1-yl)-1-methylurea; Enantiomer II | 1.4 |
| 181 | 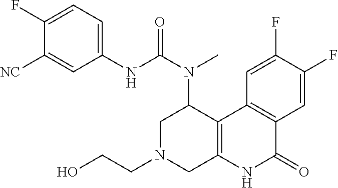 [SMILES: CN(C(=O)Nc1ccc(F)c(C#N)c1)C1CN(CCO)Cc2[nH]c(=O)c3cc(F)c(F)cc3c21] 3-(3-Cyano-4-fluorophenyl)-1-(8,9-difluoro-3-(2- hydroxyethyl)-6-oxo-1,2,3,4,5,6-hexahydrobenzo[c][1,7] 329aphthyridine-1-yl)-1-methylurea; Enantiomer I | 25 |
| 182 | 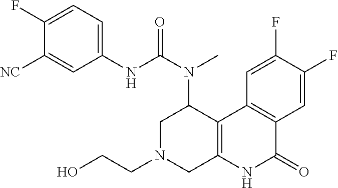 [SMILES: CN(C(=O)Nc1ccc(F)c(C#N)c1)C1CN(CCO)Cc2[nH]c(=O)c3cc(F)c(F)cc3c21] 3-(3-Cyano-4-fluorophenyl)-1-(8,9-difluoro-3-(2- hydroxyethyl)-6-oxo-1,2,3,4,5,6-hexahydrobenzo[c][1,7] naphthyridin-1-yl)-1-methylurea; Enantiomer II | 4.9 |
| 183 | 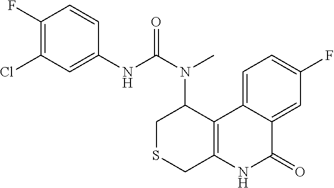 [SMILES: CN(C(=O)Nc1ccc(F)c(Cl)c1)C1CSCc2[nH]c(=O)c3cc(F)ccc3c21] 3-(3-Chloro-4-fluorophenyl)-1-(8-fluoro-6-oxo-1,4,5,6- tetrahydro-2H-thiopyrano[3,4-c]isoquinolin-1-yl)-1- methylurea; Enantiomer I | 1.1 |
| 184 | 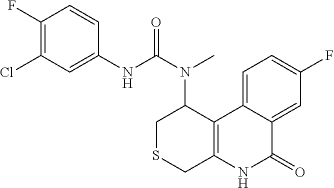 [SMILES: CN(C(=O)Nc1ccc(F)c(Cl)c1)C1CSCc2[nH]c(=O)c3cc(F)ccc3c21] 3-(3-Chloro-4-fluorophenyl)-1-(8-fluoro-6-oxo-1,4,5,6- tetrahydro-2H-thiopyrano[3,4-c]isoquinoIin-1-yl)-1- methylurea; Enantiomer II | 0.03 |
| 185 | 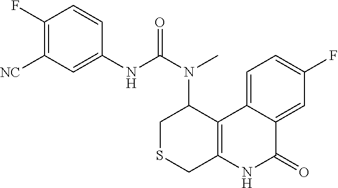 [SMILES: CN(C(=O)Nc1ccc(F)c(C#N)c1)C1CSCc2[nH]c(=O)c3cc(F)ccc3c21] 3-(3-Cyano-4-fluorophenyl)-1-(8-fluoro-6-oxo-1,4,5,6- tetrahydro-2H-thiopyrano[3,4-c]isoquinolin-1-yl)-1- methylurea; Enantiomer I | 3.3 |
| 186 | 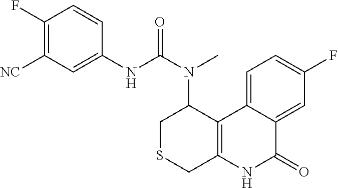 [SMILES: CN(C(=O)Nc1ccc(F)c(C#N)c1)C1CSCc2[nH]c(=O)c3cc(F)ccc3c21] 3-(3-Cyano-4-fluorophenyl)-1-(8-fluoro-6-oxo-1,4,5,6- tetrahydro-2H-thiopyrano[3,4-c]isoquinolin-1-yl)-1- methylurea; Enantiomer II | 0.06 |
| 187 | 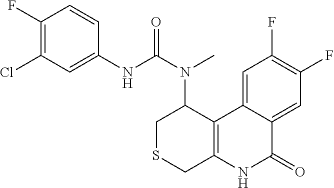 [SMILES: CN(C(=O)Nc1ccc(F)c(Cl)c1)C1CSCc2[nH]c(=O)c3cc(F)c(F)cc3c21] 3-(3-Chloro-4-fluorophenyl)-1-(8,9-difluoro-6-oxo-1,4,5,6- tetrahydro-2H-thiopyrano[3,4-c]isoquinolin-1-yl)-1- methylurea; Enantiomer I | 0.90 |
| 188 | 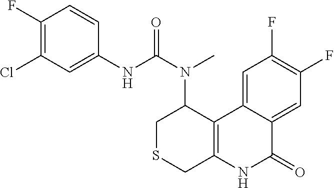 [SMILES: CN(C(=O)Nc1ccc(F)c(Cl)c1)C1CSCc2[nH]c(=O)c3cc(F)c(F)cc3c21] 3-(3-Chloro-4-fluorophenyl)-1-(8,9-difluoro-6-oxo-1,4,5,6- tetrahydro-2H-thiopyrano[3,4-c]isoquinolin-1-yl)-1- methylurea; Enantiomer II | 0.02 |
| 189 | 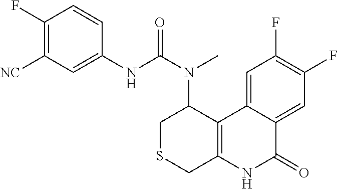 [SMILES: CN(C(=O)Nc1ccc(F)c(C#N)c1)C1CSCc2[nH]c(=O)c3cc(F)c(F)cc3c21] 3-(3-Cyano-4-fluorophenyl)-1-(8,9-difluoro-6-oxo-1,4,5,6- tetrahydro-2H-thiopyrano[3,4-c]isoquinolin-1-yl)-1-methylurea; Enantiomer I | 0.88 |
| 190 | 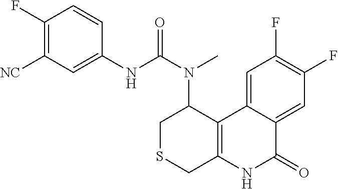 [SMILES: CN(C(=O)Nc1ccc(F)c(C#N)c1)C1CSCc2[nH]c(=O)c3cc(F)c(F)cc3c21] 3-(3-Cyano-4-fluorophenyl)-1-(8,9-difluoro-6-oxo-1,4,5,6- tetrahydro-2H-thiopyrano[3,4-c]isoquinolin-1-yl)-1- methylurea; Enantiomer II | 0.02 |
| 191 | 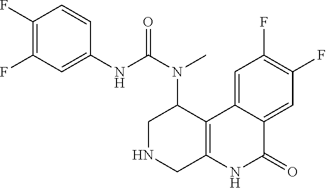 [SMILES: CN(C(=O)Nc1ccc(F)c(F)c1)C1CNCc2[nH]c(=O)c3cc(F)c(F)cc3c21] 1-(8,9-Difluoro-6-oxo-1,2,3,4,5,6- hexahydrobenzo[c][1,7]naphthyridin-1-yl)-3-(3,4- difIuorophenyl)-1-methylurea; Enantiomer I | 3.0 |
| 192 | 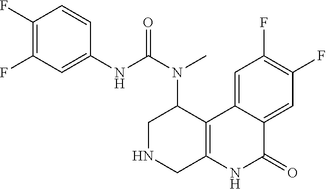 [SMILES: CN(C(=O)Nc1ccc(F)c(F)c1)C1CNCc2[nH]c(=O)c3cc(F)c(F)cc3c21] 1-(8,9-Difluoro-6-oxo-1,2,3,4,5,6- hexahydrobenzo[c][1,7]naphthyridin-1-yl)-3-(3,4- difluorophenyl)-methylurea; Enantiomer II | 0.04 |
| 193 | 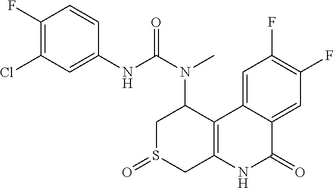 [SMILES: CN(C(=O)Nc1ccc(F)c(Cl)c1)C1CS(=O)Cc2[nH]c(=O)c3cc(F)c(F)cc3c21] 3-(3-Chloro-4-fluorophenyl)-1-(8-fluoro-3-oxido-6-oxo- 1,4,5,6-tetrahydro-2H-thiopyrano[3,4-c]isoquinolin-1-yl)- 1-methylurea; Stereoisomer 1 | 4.4 |
| 194 | 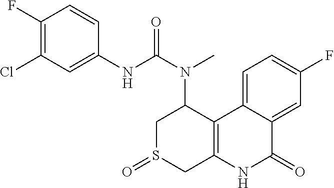 [SMILES: CN(C(=O)Nc1ccc(F)c(Cl)c1)C1CS(=O)Cc2[nH]c(=O)c3cc(F)ccc3c21] 3-(3-Chloro-4-fluorophenyl)-1-(8-fluoro-3-oxido-6-oxo- 1,4,5,6-tetrahydro-2H-thiopyrano[3,4-c]isoquinolin-1- yl)-1-methylurea; Stereoisomer II (Enantiomer of I) | 25 |
| 195 | 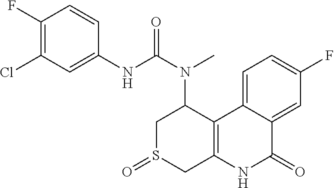 [SMILES: CN(C(=O)Nc1ccc(F)c(Cl)c1)C1CS(=O)Cc2[nH]c(=O)c3cc(F)ccc3c21] 3-(3-Chloro-4-fluorophenyl)-]-(8-fluoro-3-oxido-6-oxo- 1,4,5,6-tetrahydro-2H-thiopyrano[3,4-c]isoquinolin-1- yl)-1-methylurea; Stereoisomer III | 0.90 |
| 196 | 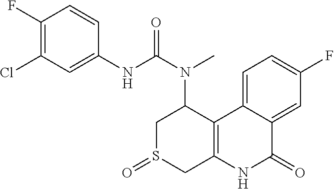 [SMILES: CN(C(=O)Nc1ccc(F)c(Cl)c1)C1CS(=O)Cc2[nH]c(=O)c3cc(F)ccc3c21] 3-(3-Chloro-4-fluorophenyl)-1-(8-fluoro-3-oxido-6-oxo- 1,4,5,6-tetrahydro-2H-thiopyrano[3,4-c]isoquinolin-1- yl)-1-methylurea; Stereoisomer IV (Enantiomer of III) | 22 |
| 197 | 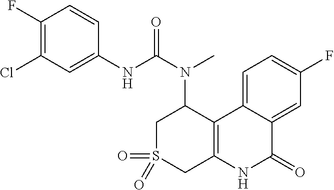 [SMILES: CN(C(=O)Nc1ccc(F)c(Cl)c1)C1CS(=O)(=O)Cc2[nH]c(=O)c3cc(F)ccc3c21] 3-(3-Chloro-4-fluorophenyl)-1-(8-fluoro-3,3-dioxido-6- oxo-1,4,5,6-tetrahydro-2H-thiopyrano[3,4-c]isoquinolin-1- yl)-1-methylurea; Enantiomer I | 2.3 |
| 198 | 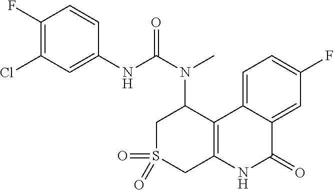 [SMILES: CN(C(=O)Nc1ccc(F)c(Cl)c1)C1CS(=O)(=O)Cc2[nH]c(=O)c3cc(F)ccc3c21] 3-(3-Chloro-4-fluorophenyl)-1-(8-fluoro-3,3-dioxido-6- oxo-1,4,5,6-tetrahydro-2H-thiopyrano[3,4-c]isoquinolin-1- yl)-1-methylurea; Enantiomer II | 3.7 |
| 199 | 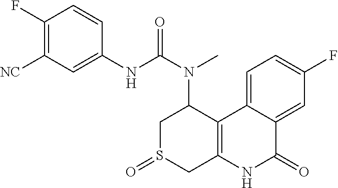 [SMILES: CN(C(=O)Nc1ccc(F)c(C#N)c1)C1CS(=O)Cc2[nH]c(=O)c3cc(F)ccc3c21] 3-(3-Cyano-4-fluorophenyl)-1-(8-fluoro-3-oxido-6-oxo- 1,4,5,6-tetrahydro-2H-thiopyrano[3,4-c]isoquinolin-1- yl)-1-methylurea; Stereoisomer I | 20 |
| 200 | 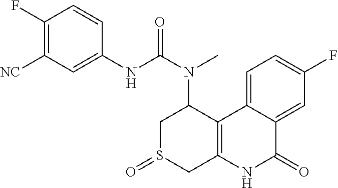 [SMILES: CN(C(=O)Nc1ccc(F)c(C#N)c1)C1CS(=O)Cc2[nH]c(=O)c3cc(F)ccc3c21] 3-(3-Cyano-4-fluorophenyl)-1-(8-fluoro-3-oxido-6-oxo- 1,4,5,6-tetrahydro-2H-thiopyrano[3,4-c]isoquinolin-1- yl)-1-methylurea; Stereoisomer II (Enantiomer of I) | 25 |
| 201 | 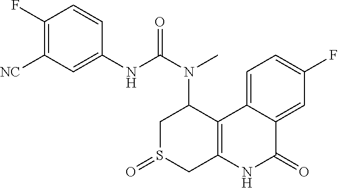 [SMILES: CN(C(=O)Nc1ccc(F)c(C#N)c1)C1CS(=O)Cc2[nH]c(=O)c3cc(F)ccc3c21] 3-(3-Cyano-4-fluorophenyl)-1-(8-fluoro-3-oxido-6-oxo-1,4,5,6- tetrahydro-2H-thiopyrano[3,4-c]isoquinolin-1-yl)-1-methylurea; Stereoisomer III | 3.6 |
| 202 | 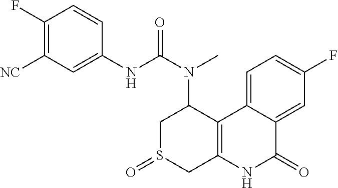 [SMILES: CN(C(=O)Nc1ccc(F)c(C#N)c1)C1CS(=O)Cc2[nH]c(=O)c3cc(F)ccc3c21] 3-(3-Cyano-4-fluorophenyl)-1-(8-fluoro-3-oxido-6-oxo- 1,4,5,6-tetrahydro-2H-thiopyrano[3,4-c]isoquinolin-1- yl)-1-methylurea; Stereoisomer IV (Enantiomer of III) | 25 |
| 203 | 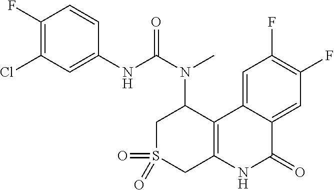 [SMILES: CN(C(=O)Nc1ccc(F)c(Cl)c1)C1CS(=O)(=O)Cc2[nH]c(=O)c3cc(F)c(F)cc3c21] 3-(3-Chloro-4-fluorophenyl)-1-(8,9-difluoro-3,3-dioxido-6- oxo-1,4,5,6-tetrahydro-2H-thiopyrano[3,4-c]isoquinolin-1- yl)-1-methylurea; Enantiomer I | 1.4 |
| 204 | 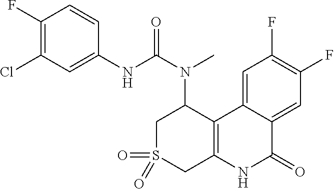 [SMILES: CN(C(=O)Nc1ccc(F)c(Cl)c1)C1CS(=O)(=O)Cc2[nH]c(=O)c3cc(F)c(F)cc3c21] 3-(3-Chloro-4-fluorophenyl)-1-(8,9-difluoro-3,3-dioxido-6- oxo-1,4,5,6-tetrahydro-2H-thiopyrano[3,4-c]isoquinolin-1- yl)-1-methylurea; Enantiomer II | 1.4 |
| 205 | 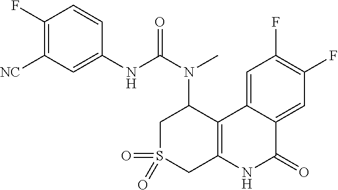 [SMILES: CN(C(=O)Nc1ccc(F)c(C#N)c1)C1CS(=O)(=O)Cc2[nH]c(=O)c3cc(F)c(F)cc3c21] 3-(3-Cyano-4-fluorophenyl)-1-(8,9-difluoro-3,3-dioxido-6- oxo-1,4,5,6-tetrahydro-2H-thiopyrano[3,4-c]isoquinolin-1- yl)-1-methylurea; Enantiomer I | 7.2 |
| 206 | 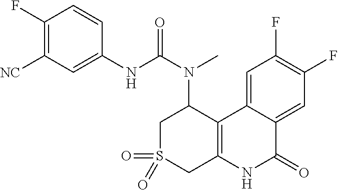 [SMILES: CN(C(=O)Nc1ccc(F)c(C#N)c1)C1CS(=O)(=O)Cc2[nH]c(=O)c3cc(F)c(F)cc3c21] 3-(3-Cyano-4-fluorophenyl)-1-(8,9-difluoro-3,3-dioxido-6- oxo-1,4,5,6-tetrahydro-2H-thiopyrano[3,4-c]isoquinolin-1- yl)-1-methylurea; Enantiomer II | 4.8 |
| 207 | 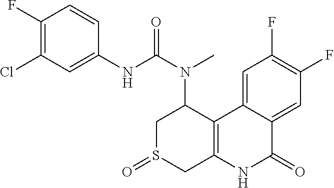 [SMILES: CN(C(=O)Nc1ccc(F)c(Cl)c1)C1CS(=O)Cc2[nH]c(=O)c3cc(F)c(F)cc3c21] 3-(3-Chloro-4-fluorophenyl)-1-(8,9-difluoro-3-oxido-6-oxo- 1,4,5,6-tetrahydro-2H-thiopyrano[3,4-c]isoquinolin-1-yl)-1- methylurea; Stereoisomer I | 1.3 |
| 208 | 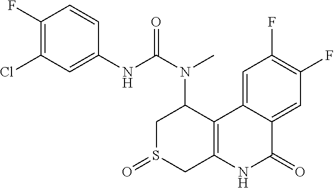 [SMILES: CN(C(=O)Nc1ccc(F)c(Cl)c1)C1CS(=O)Cc2[nH]c(=O)c3cc(F)c(F)cc3c21] 3-(3-Chioro-4-fluorophenyl)-1-(8,9-difluoro-3-oxido-6- oxo-1,4,5,6-tetrahydro-2H-thiopyrano[3,4-c]isoquinolin-1- yl)-1-methylurea; Stereoisomer II (Enantiomer of I) | 8.5 |
| 209 | 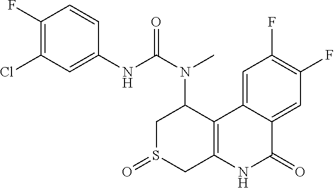 [SMILES: CN(C(=O)Nc1ccc(F)c(Cl)c1)C1CS(=O)Cc2[nH]c(=O)c3cc(F)c(F)cc3c21] 3-(3-Chloro-4-fluorophenyl)-1-(8,9-difluoro-3-oxido-6- oxo-1,4,5,6-tetrahydro-2H-thiopyrano[3,4-c]isoquinolin-1- yl)-1-methylurea; Stereoisomer III | 0.70 |
| 210 | 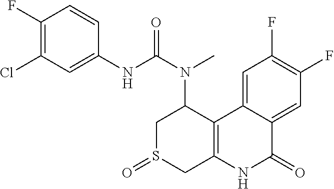 [SMILES: CN(C(=O)Nc1ccc(F)c(Cl)c1)C1CS(=O)Cc2[nH]c(=O)c3cc(F)c(F)cc3c21] 3-(3-Chloro-4-fluorophenyl)-1-(8,9-difluoro-3-oxido-6- oxo-1,4,5,6-tetrahydro-2H-thiopyrano[3,4-c]isoquinolin-1- yl)-1-methylurea; Stereoisomer IV (Enantiomer of III) | 7.1 |
| 211 | 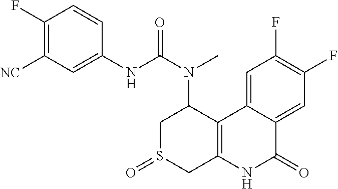 [SMILES: CN(C(=O)Nc1ccc(F)c(C#N)c1)C1CS(=O)Cc2[nH]c(=O)c3cc(F)c(F)cc3c21] 3-(3-Cyano-4-fluorophenyl)-1-(8,9-difluoro-3-oxido-6- oxo-1,4,5,6-tetrahydro-2H-thiopyrano[3,4-c]isoquinolin-1- yl)-1-methylurea; Stereoisomer I | 5.5 |
| 212 | 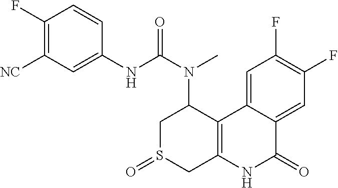 [SMILES: CN(C(=O)Nc1ccc(F)c(C#N)c1)C1CS(=O)Cc2[nH]c(=O)c3cc(F)c(F)cc3c21] 3-(3-Cyano-4-fluorophenyl)-1-(8,9-difluoro-3-oxido-6-oxo- 1,4,5,6-tetrahydro-2H-thiopyrano[3,4-c]isoquinolin-1- yl)-1-methylurea; Stereoisomer II (Enantiomer of I) | 25 |
| 213 | 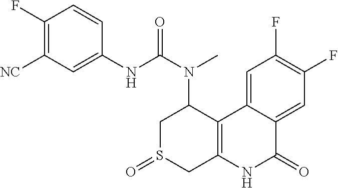 [SMILES: CN(C(=O)Nc1ccc(F)c(C#N)c1)C1CS(=O)Cc2[nH]c(=O)c3cc(F)c(F)cc3c21] 3-(3-Cyano-4-fluorophenyl)-1-(8,9-difluoro-3-oxido-6-oxo- 1,4,5,6-tetrahydro-2H-thiopyrano[3,4-c]isoquinolin-1- yl)-1-methylurea; Stereoisomer III | 1.0 |
| 214 | 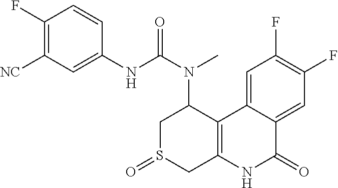 [SMILES: CN(C(=O)Nc1ccc(F)c(C#N)c1)C1CS(=O)Cc2[nH]c(=O)c3cc(F)c(F)cc3c21] 3-(3-Cyano-4-fluorophenyl)-1-(8,9-difluoro-3-oxido-6-oxo- 1,4,5,6-tetrahydro-2H-thiopyrano[3,4-c]isoquinolin-1- yl)-1-methylurea; Stereoisomer IV (Enantiomer of III) | 15 |
| 215 | 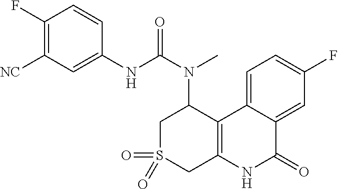 [SMILES: CN(C(=O)Nc1ccc(F)c(C#N)c1)C1CS(=O)(=O)Cc2[nH]c(=O)c3cc(F)ccc3c21] 3-(3-Cyano-4-fluorophenyl)-1-(8-fluoro-3,3-dioxido-6-oxo- 1,4,5,6-tetrahydro-2H-thiopyrano[3,4-c]isoquinolin-1- yl)-1-methylurea | 25 |
| 216 | 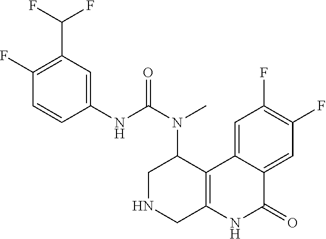 [SMILES: CN(C(=O)Nc1ccc(F)c(C(F)F)c1)C1CNCc2[nH]c(=O)c3cc(F)c(F)cc3c21] 1-(8,9-difluoro-6-oxo-1,2,3,4,5,6- hexahydrobenzo[c][1,7]naphthyridin-1-yl)-3-(3- (difluoromethyl)-4-fluorophenyl)-1-methylurea; Enantiomer I | 3.5 |
| 217 | 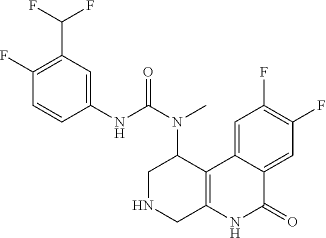 [SMILES: CN(C(=O)Nc1ccc(F)c(C(F)F)c1)C1CNCc2[nH]c(=O)c3cc(F)c(F)cc3c21] 1-(8,9-difluoro-6-oxo-1,2,3,4,5,6- hexahydrobenzo[c][1,7]naphthyridin-1-yl)-3-(3- (difluoromethyl)-4-fluorophenyl)-1-methylurea; Enantiomer II | 0.04 |
| 218 | 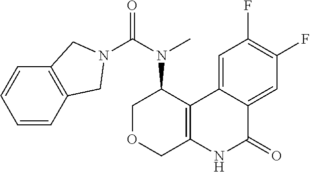 [SMILES: CN(C(=O)N1Cc2ccccc2C1)[C@@H]1COCc2[nH]c(=O)c3cc(F)c(F)cc3c21] (S)-N-(8,9-difluoro-6-oxo-1,4,5,6-tetrahydro-2H- pyrano[3,4-c]isoquinolin-1-yl)-N-methylisoindoline- 2-carboxainide | 1.2 |
| 219 | 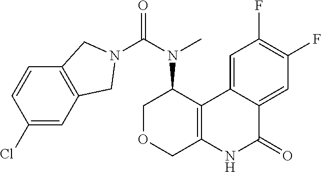 [SMILES: CN(C(=O)N1Cc2ccc(Cl)cc2C1)[C@@H]1COCc2[nH]c(=O)c3cc(F)c(F)cc3c21] (S)-5-chloro-N-(8,9-difluoro-6-oxo-1,4,5,6-tetrahydro-2H- pyrano[3,4-c]isoquinolin-1-yl)-N-methylisoindoline-2- carboxamide | 5.9 |
| 220 | 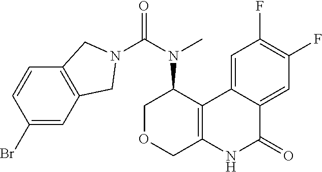 [SMILES: CN(C(=O)N1Cc2ccc(Br)cc2C1)[C@@H]1COCc2[nH]c(=O)c3cc(F)c(F)cc3c21] (S)-5-bromo-N-(8,9-difluoro-6-oxo-1,4,5,6-tetrahydro- 2H-pyrano[3,4-c]isoquinolin-1-yl)-N-methylisoindoline- 2-carboxamide | 13 |
| 221 | 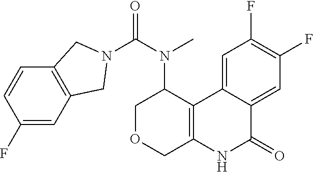 [SMILES: CN(C(=O)N1Cc2ccc(F)cc2C1)C1COCc2[nH]c(=O)c3cc(F)c(F)cc3c21] (S)-5-fluoro-N-(8,9-difluoro-6-oxo-1,4,5,6-tetrahydro- 2H-pyrano[3,4-c]isoquinolin-1-yl)-N- methylisoindoline-2-carboxamide | 5.9 |
| 222 | 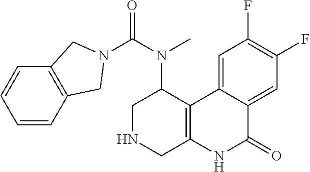 [SMILES: CN(C(=O)N1Cc2ccccc2C1)C1CNCc2[nH]c(=O)c3cc(F)c(F)cc3c21] N-(8,9-difluoro-6-oxo-1,2,3,4,5,6- hexahydrobenzo[c][1,7]naphthyridin-1-yl)-N- methylisoindoline-2-carboxamide; Enantiomer II | 25 |
| 223 | 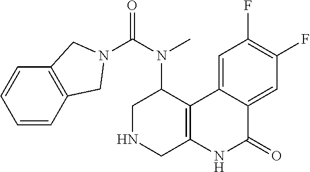 [SMILES: CN(C(=O)N1Cc2ccccc2C1)C1CNCc2[nH]c(=O)c3cc(F)c(F)cc3c21] N-(8,9-difluoro-6-oxo-1,2,3,4,5,6- hexahydrobenzo[c][1,7]naphthyridin-1-yl)-N- methylisoindoline-2-carboxamide; Enantiomer I | 2.3 |
| 224 | 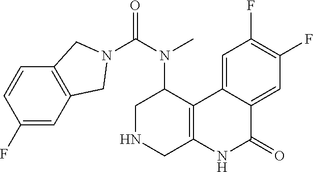 [SMILES: CN(C(=O)N1Cc2ccc(F)cc2C1)C1CNCc2[nH]c(=O)c3cc(F)c(F)cc3c21] N-(8,9-difluoro-6-oxo-1,2,3,4,5,6- hexahydrobenzo[c][1,7]naphthyridin-1-yl)-5-fluoro-N- methylisoindoline-2-carboxamide; Enantiomer II | 25 |
| 225 | 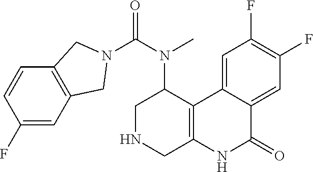 [SMILES: CN(C(=O)N1Cc2ccc(F)cc2C1)C1CNCc2[nH]c(=O)c3cc(F)c(F)cc3c21] N-(8,9-difluoro-6-oxo-1,2,3,4,5,6- hexahydrobenzo[c][1,7]naphthyridin-1-yl)-5-fluoro-N- methylisoindoline-2-carboxamide; Enantiomer I | 3.4 |
| 226 | 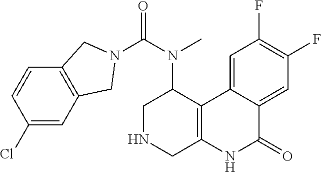 [SMILES: CN(C(=O)N1Cc2ccc(Cl)cc2C1)C1CNCc2[nH]c(=O)c3cc(F)c(F)cc3c21] N-(8,9-difluoro-6-oxo-1,2,3,4,5,6- hexahydrobenzo[c][1,7]naphthyridin-1-yl)-5-chloro-N- methylisoindoline-2-carboxamide.; Enantiomer I | 21 |
| 227 | 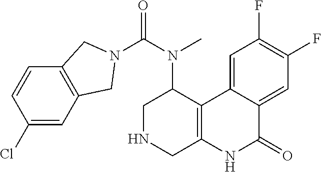 [SMILES: CN(C(=O)N1Cc2ccc(Cl)cc2C1)C1CNCc2[nH]c(=O)c3cc(F)c(F)cc3c21] N-(8,9-difluoro-6-oxo-1,2,3,4,5,6- hexahydrobenzo[c][1,7]naphthyridin-1-yl)-5-chloro- N-methylisoindoline-2-carboxamide; Enantiomer II | 12 |
| 228 | 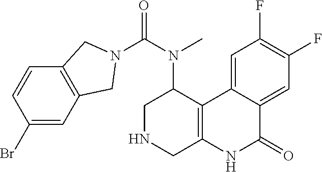 [SMILES: CN(C(=O)N1Cc2ccc(Br)cc2C1)C1CNCc2[nH]c(=O)c3cc(F)c(F)cc3c21] N-(8,9-difluoro-6-oxo-1,2,3,4,5,6- hexahydrobenzo[c][1,7]naphthyridin-1-yl)-5- bromo-N-methylisoindoline-2-carboxamide, Enantiomer I | 15 |
| 229 | 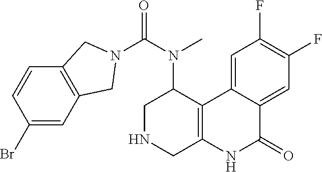 [SMILES: CN(C(=O)N1Cc2ccc(Br)cc2C1)C1CNCc2[nH]c(=O)c3cc(F)c(F)cc3c21] N-(8,9-difluoro-6-oxo-1,2,3,4,5,6- hexahydrobenzo[c][1,7]naphthyridin-1-yl)-5-bromo- N-methylisoindoline-2-carboxamide; Enantiomer II | 6.7 |
| 230 | 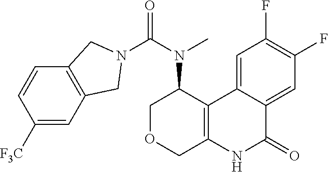 [SMILES: CN(C(=O)N1Cc2ccc(C(F)(F)F)cc2C1)[C@@H]1COCc2[nH]c(=O)c3cc(F)c(F)cc3c21] (S)-N-(8,9-difluoro-6-oxo-1,4,5,6-tetrahydro-2H-pyrano [3,4-c]isoquinolin-1-yl)-N-methyl-5-(trifluoromethyl) isoindoline-2-carboxamide | 24 |
| 231 | 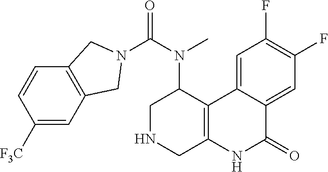 [SMILES: CN(C(=O)N1Cc2ccc(C(F)(F)F)cc2C1)C1CNCc2[nH]c(=O)c3cc(F)c(F)cc3c21] N-(8,9-difluoro-6-oxo-1,2,3,4,5,6- hexahydrobenzo[c][1,7]naphthyridin-1-yl)-N-methyl-5- (trifluoromethyl)isoindoline-2-carboxamide; Enantiomer I | 25 |
| 232 | 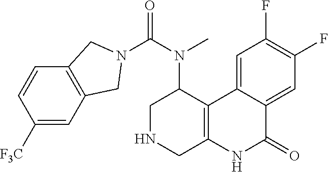 [SMILES: CN(C(=O)N1Cc2ccc(C(F)(F)F)cc2C1)C1CNCc2[nH]c(=O)c3cc(F)c(F)cc3c21] N-(8,9-difluoro-6-oxo-1,2,3,4,5,6- hexahydrobenzo[c][1,7]naphthyridin-1-yl)-N-methyl-5- (trifluoromethyl)isoindoline-2-carboxamide; Enantiomer II | 20 |
| 233 | 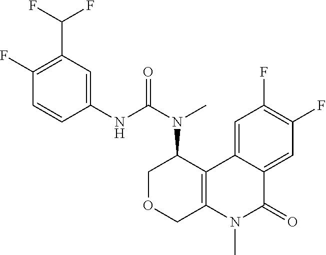 [SMILES: CN(C(=O)Nc1ccc(F)c(C(F)F)c1)[C@@H]1COCc2c1c1cc(F)c(F)cc1c(=O)n2C] (S)-1-(8,9-difluoro-5-methyl-6-oxo-1,4,5,6-tetrahydro- 2H-pyrano[3,4-c]isoquinolin-1-yl)-3-(3- (difluoromethyl)-4-fluorophenyl)-1-methylurea | 0.02 |
| 234 | 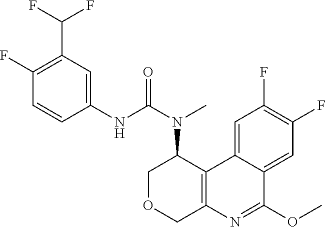 [SMILES: COc1nc2c(c3cc(F)c(F)cc13)[C@H](N(C)C(=O)Nc1ccc(F)c(C(F)F)c1)COC2] (S)-1-(8,9-difluoro-6-methoxy-1,4-dihydro-2H-pyrano [3,4-c]isoquinolin-1-yl)-3-(3-(difluoromethyl)-4- fluorophenyl)-1-methylurea | 0.05 |
| 235 | 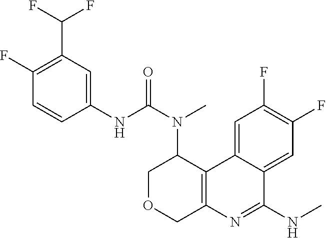 [SMILES: CNc1nc2c(c3cc(F)c(F)cc13)C(N(C)C(=O)Nc1ccc(F)c(C(F)F)c1)COC2] 1-(8,9-difluoro-6-(methylamino)-1,4-dihydro-2H-pyrano [3,4-c]isoquinolin-1-yl)-3-(3-(difluoromethyl)-4- fluorophenyl)-1-methylurea; Enantiomer I | 1.81 |
| 236 | 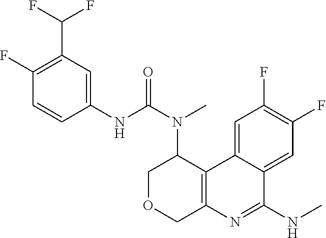 [SMILES: CNc1nc2c(c3cc(F)c(F)cc13)C(N(C)C(=O)Nc1ccc(F)c(C(F)F)c1)COC2] 1-(8,9-difluoro-6-(methylamino)-1,4-dihydro-2H-pyrano [3,4-c]isoquinolin-1-yl)-3-(3-(difluoromethyl)-4- fluorophenyl)-1-methylurea; Enantiomer II | 0.01 |
| 237 | 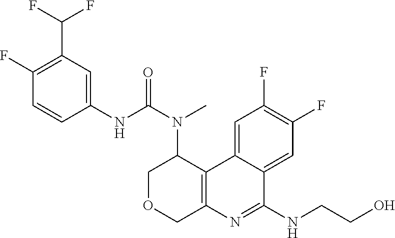 [SMILES: CN(C(=O)Nc1ccc(F)c(C(F)F)c1)C1COCc2nc(NCCO)c3cc(F)c(F)cc3c21] 1-(8,9-difluoro-6-((2-hydroxyethyl)amino)-1,4-dihydro-2H- pyrano[3,4-c]isoquinolin-1-yl)-3-(3-(difluoromethyl)-4- fluorophenyl)-1-methylurea; Enantiomer I | 2.62 |
| 238 | 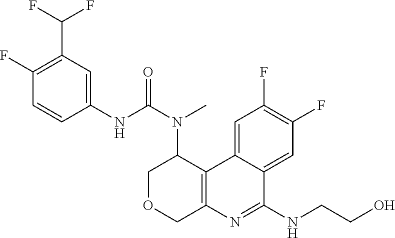 [SMILES: CN(C(=O)Nc1ccc(F)c(C(F)F)c1)C1COCc2nc(NCCO)c3cc(F)c(F)cc3c21] 1-(8,9-difluoro-6-((2-hydroxyethyl)amino)-1,4-dihydro-2H- pyrano[3,4-c]isoquinolin-1-yl)-3-(3-(difluoromethyl)-4- fluorophenyl)-1-methylurea; Enantiomer II | 0.02 |
| 239 | 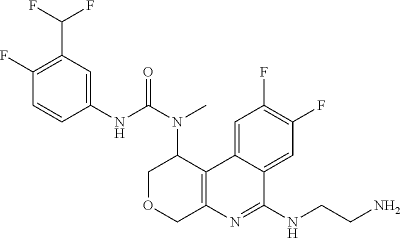 [SMILES: CN(C(=O)Nc1ccc(F)c(C(F)F)c1)C1COCc2nc(NCCN)c3cc(F)c(F)cc3c21] 1-(8,9-difluoro-6-((2-aminoethyl)amino)-1,4-dihydro-2H- pyrano[3,4-c]isoquinolin-1-yl)-3-(3-(difluoromethyI)-4- fluorophenyl)-1-methylurea | 0.30 |
| 240 | 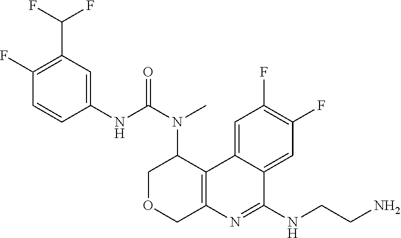 [SMILES: CN(C(=O)Nc1ccc(F)c(C(F)F)c1)C1COCc2nc(NCCN)c3cc(F)c(F)cc3c21] 3-(3-chloro-4-fluorophenyl)-1-(8,9-difluoro-6-((2- aminoethyl)amino)-1,4-dihydro-2H-pyrano[3,4-c]isoquinolin-1- yl)-1-methylurea | 0.59 |
| 241 | 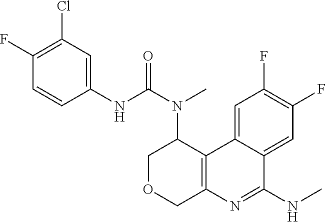 [SMILES: CNc1nc2c(c3cc(F)c(F)cc13)C(N(C)C(=O)Nc1ccc(F)c(Cl)c1)COC2] 3-(3-chloro-4-fluorophenyl)-1-(8,9-difluoro-6- (methylarnino)-1,4-dihydro-2H-pyrano[3,4-c] isoquinolin-1-yl)-1-methylurea; Enantiomer I | 2.38 |
| 242 | 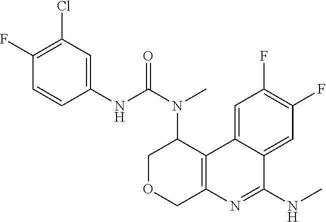 [SMILES: CNc1nc2c(c3cc(F)c(F)cc13)C(N(C)C(=O)Nc1ccc(F)c(Cl)c1)COC2] 3-(3-chloro-4-fluorophenyl)-1-(8,9-difluoro-6- (methylamino)-1,4-dihydro-2H-pyrano[3,4-c] isoquinolin-1-yl)-1-methylurea; Enantiomer II | 0.02 |
| 243 | 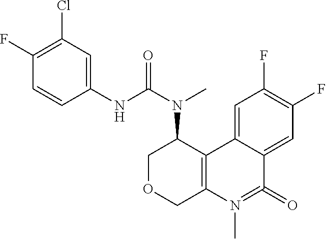 (S)-1-(8,9-difIuoro-5-methyl-6-oxo-1,4,5,6-tetrahydro- 2H-pyrano[3,4-c]isoquinolin-1-yl)-3-(3-chloro-4- fluorophenyl)-1-methylurea | 0.02 |
| 244 | 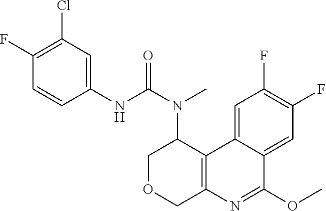 [SMILES: COc1nc2c(c3cc(F)c(F)cc13)C(N(C)C(=O)Nc1ccc(F)c(Cl)c1)COC2] (S)-3-(3-chloro-4-fluorophenyl)-1-(8,9-difluoro-6- methoxy-1,4-dihydro-2H-pyrano[3,4-c] isoquinolin-1-yl)-1-methylurea | 0.08 |
| 245 | 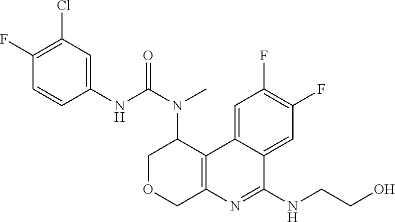 [SMILES: CN(C(=O)Nc1ccc(F)c(Cl)c1)C1COCc2nc(NCCO)c3cc(F)c(F)cc3c21] 3-(3-chloro-4-fluorophenyl)-1-(8,9-difluoro-6-((2- hydroxyethyl)amino)-1,4-dihydro-2H-pyrano[3,4-c]isoquinolin- l-yl)-1-methylurea; Enantiomer I | 4.20 |
| 246 | 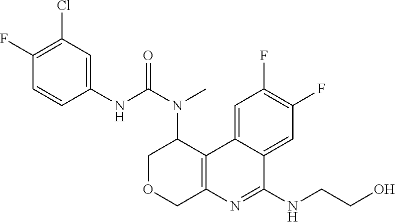 [SMILES: CN(C(=O)Nc1ccc(F)c(Cl)c1)C1COCc2nc(NCCO)c3cc(F)c(F)cc3c21] 3-(3-chloro-4-fluorophenyl)-1-(8,9-difluoro-6-((2- hydroxyethyl)amino)-1,4-dihydro-2H-pyrano[3,4-c] isoquinolin-1-yl)-1-methylurea; Enantiomer II | 0.02 |
| 247 | 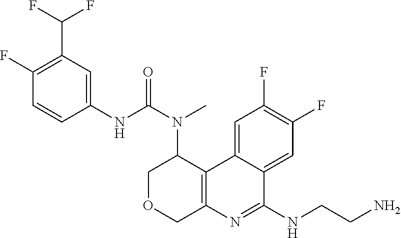 [SMILES: CN(C(=O)Nc1ccc(F)c(C(F)F)c1)C1COCc2nc(NCCN)c3cc(F)c(F)cc3c21] 1-(8,9-difluoro-6-((2-aminoethyl)amino)-1,4-dihydro- 2H-pyrano[3,4-c]isoquinolin-1-yl)-3-(3-(difluoromethyl)-4- fluorophenyl)-1-methylurea; Enantiomer I | 0.08 |
| 248 | 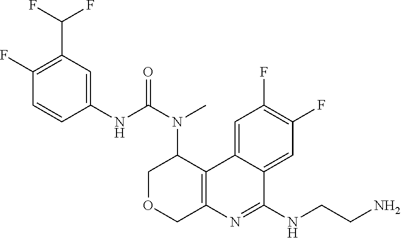 [SMILES: CN(C(=O)Nc1ccc(F)c(C(F)F)c1)C1COCc2nc(NCCN)c3cc(F)c(F)cc3c21] 1-(8,9-difluoro-6-((2-aminoethyl)amino)-1,4-dihydro-2H-pyrano [3,4-c]isoquinolin-1-yl)-3-(3-(difluoromethyl)-4-fluorophenyl)-1- methylurea; Enantiomer II | 3.20 |
| 249 | 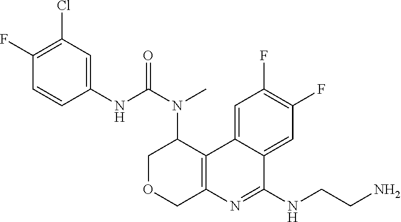 [SMILES: CN(C(=O)Nc1ccc(F)c(Cl)c1)C1COCc2nc(NCCN)c3cc(F)c(F)cc3c21] 3-(3-chloro-4-fluorophenyl)-1-(8,9-difluoro-6-((2- aminoethyl)amino)-1,4-dihydro-2H-pyrano[3,4-c] isoquinolin-1-yl)-1-methylurea; Enantiomer I | 8.30 |
| 250 | 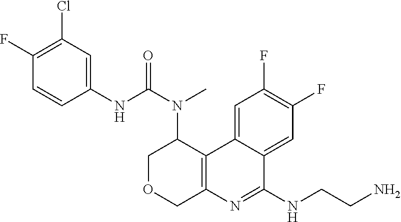 [SMILES: CN(C(=O)Nc1ccc(F)c(Cl)c1)C1COCc2nc(NCCN)c3cc(F)c(F)cc3c21] 3-(3-chloro-4-fluorophenyl)-1-(8,9-difluoro-6-((2- aminoethyl)amino)-1,4-dihydro-2H-pyrano[3,4-c] isoquinolin-1-yl)-1-methylurea; Enantiomer II | 0.27 |
| 251 | 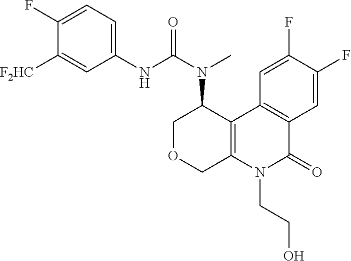 [SMILES: CN(C(=O)Nc1ccc(F)c(C(F)F)c1)[C@@H]1COCc2c1c1cc(F)c(F)cc1c(=O)n2CCO] (S)-1-(8,9-Difluoro-5-(2-hydroxyethyl)-6-oxo-1,4,5,6- tetrahydro-2H-pyrano[3,4-c]isoquinolin-1-yl)-3-(3- (difluoromethyl)-4-fluorophenyl)-1-methylurea | 0.030 |
| 252 | 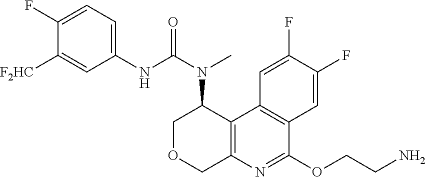 [SMILES: CN(C(=O)Nc1ccc(F)c(C(F)F)c1)[C@@H]1COCc2nc(OCCN)c3cc(F)c(F)cc3c21] (S)-1-(6-(2-Aminoethoxy)-8,9-difluoro-1,4-dihydro-2H- pyrano[3,4-c]isoquinolin-1-yl)-3-(3-(difluoromethyl)-4- fluorophenyl)-1-methylurea | 0.020 |
| 253 | 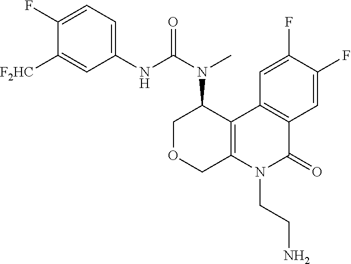 [SMILES: CN(C(=O)Nc1ccc(F)c(C(F)F)c1)[C@@H]1COCc2c1c1cc(F)c(F)cc1c(=O)n2CCN] (S)-1-(5-(2-Aminoethyl)-8,9-difluoro-6-oxo- 1,4,5,6-tetrahydro-2H-pyrano[3,4-c]isoquinolin-1- yl)-3-(3-(difluoromethyl)-4-fluorophenyl)-1-methylurea | 0.91 |
| 254 | 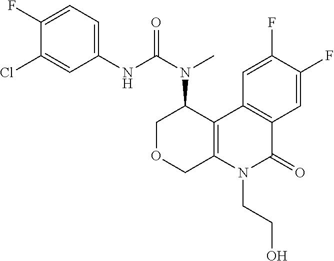 [SMILES: CN(C(=O)Nc1ccc(F)c(Cl)c1)[C@@H]1COCc2c1c1cc(F)c(F)cc1c(=O)n2CCO] (S)-3-(3-Chloro-4-fluorophenyl)-1-(8,9-difluoro-5-(2- hydroxyethyl)-6-oxo-1,4,5,6-tetrahydro-2H- pyrano[3,4-c]isoquinolin-1-yl)-1-methylurea | 0.058 |
| 255 | 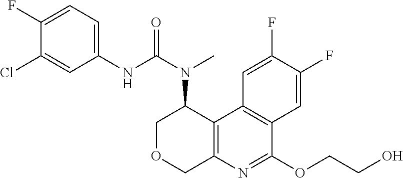 [SMILES: CN(C(=O)Nc1ccc(F)c(Cl)c1)[C@@H]1COCc2nc(OCCO)c3cc(F)c(F)cc3c21] (S)-3-(3-Chloro-4-fluorophenyl)-1-(8,9-difluoro-6-(2-hydroxyethoxy)- 1,4-dihydro-2H-pyrano[3,4-c]isoquinolin-1-yl)-1-methylurea | 0.049 |
| 256 | 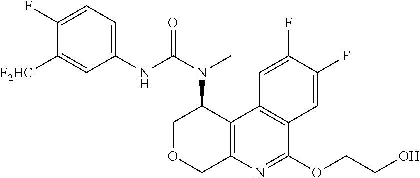 [SMILES: CN(C(=O)Nc1ccc(F)c(C(F)F)c1)[C@@H]1COCc2nc(OCCO)c3cc(F)c(F)cc3c21] (S)-1-(8,9-Difluoro-6-(2-hydroxyethoxy)-1,4-dihydro-2H-pyrano [3,4-c]isoquinolin-1-yl)-3-(3-(difluoromethyl)-4-fluorophenyl)- 1-methylurea | 0.019 |
| 257 | 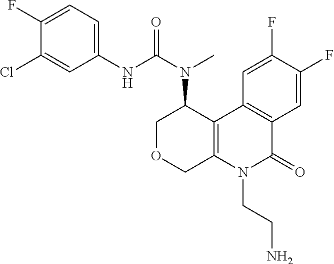 [SMILES: CN(C(=O)Nc1ccc(F)c(Cl)c1)[C@@H]1COCc2c1c1cc(F)c(F)cc1c(=O)n2CCN] (S)-1-(5-(2-Aminoethyl)-8,9-difluoro-6-oxo-1,4,5,6- tetrahydro-2H-pyrano[3,4-c]isoquinolin-1-yl)-3-(3-chloro- 4-fluorophenyl)-1-methylurea | 3.1 |
| 258 | 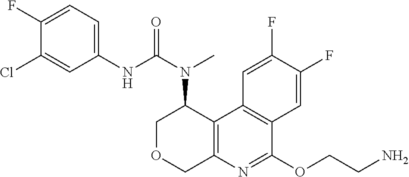 [SMILES: CN(C(=O)Nc1ccc(F)c(Cl)c1)[C@@H]1COCc2nc(OCCN)c3cc(F)c(F)cc3c21] (S)-1-(6-(2-Aminoethoxy)-8,9-difluoro-1,4-dihydro-2H-pyrano [3,4-c]isoquinolin-1-yl)-3-(3-chloro-4-fluorophenyl)-1-methylurea | 0.065 |

### Enumerated Embodiments

The following exemplary embodiments are provided, the numbering of which is not to be construed as designating levels of importance:

Embodiment 1 provides a compound of formula (I), or a salt, solvate, prodrug, stereoisomer, tautomer, or isotopically labelled derivative thereof, or any mixtures thereof:

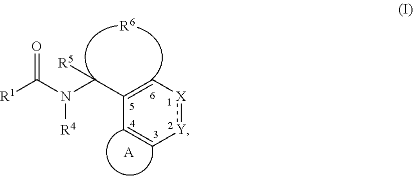

wherein:

X, Y, and the bond between X and Y are such that:
X is NR⁷, Y is C(═O), and the bond between X and Y is a single bond, or
X is N, Y is CR¹⁰, and the bond between X and Y is a double bond,

A ring

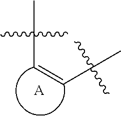

is selected from the group consisting of:

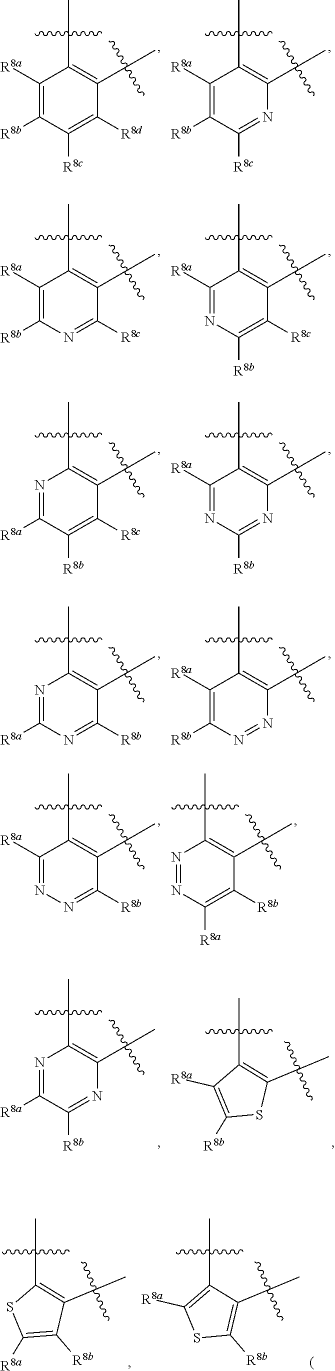

(wherein there is no bridgehead double bond),

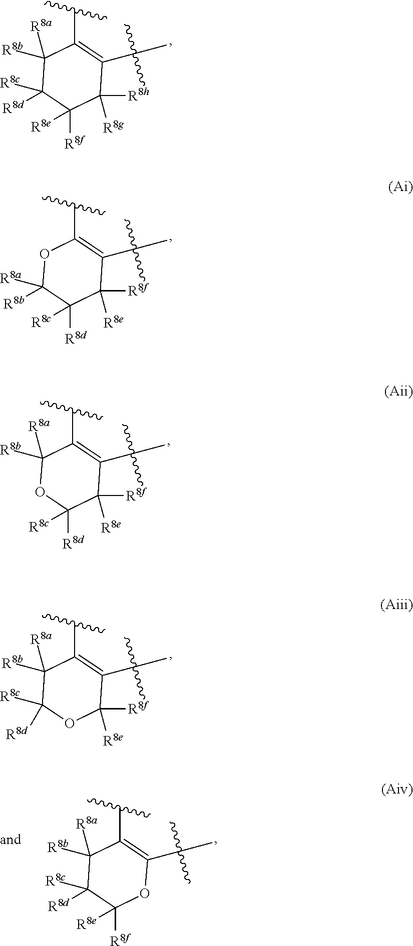

wherein: in (Ai) R^{8a} and R^{8b} optionally combine with the carbon atom to which they are attached to form carbonyl (—(C═O)—);
   in (Aii) R^{8a} and R^{8b}, or R^{8c} and R^{8d}, optionally combine with the carbon atom to which they are attached to form carbonyl (—(C═O)—);
   in (Aiii) R^{8c} and R^{8d}, or R^{8e} and R^{8f}, optionally combine with the carbon atom to which they are attached to form carbonyl (—(C═O)—);
   in (Aiv) R^{8e} and R^{8f} optionally combine with the carbon atom to which they are attached to form carbonyl (—(C═O)—);
or the A ring is absent, position 3 of the pyridin-2-one ring is substituted with R^{8a}, and position 4 of the pyridin-2-one ring is substituted with R^{8b};

R¹ is —NR²R³ or optionally substituted isoindolin-2-yl;

R² is selected from the group consisting of optionally substituted C₃-C₈ cycloalkyl, optionally substituted phenyl, optionally substituted benzyl, optionally substituted heteroaryl, and —(CH₂)(optionally substituted heteroaryl);

R³ is selected from the group consisting of H and C₁-C₆ alkyl;

R⁴ is selected from the group consisting of H, C₁-C₆ alkyl, and C₃-C₈ cycloalkyl, wherein the alkyl or cycloalkyl is optionally substituted with at least one selected from the group consisting of C₁-C₆ alkyl, C₃-C₈ cycloalkyl, halogen, cyano, —OH, C₁-C₆ alkoxy, C₃-C₈ cycloalkoxy, C₁-C₆ haloalkoxy, C₃-C₈ halocycloalkoxy, optionally substituted phenyl, optionally substituted heteroaryl, optionally substituted heterocyclyl, —C(═O)OR⁹, —OC(═O)R⁹, —SR⁹, —S(═O)R⁹, —S(═O)₂R⁹, —S(═O)₂NR⁹R⁹, —N(R⁹)S(═O)₂R⁹, —N(R⁹)C(═O)R⁹, —C(═O)NR⁹R⁹, and —NR⁹R⁹;

R⁵ is selected from the group consisting of H and optionally substituted C₁-C₆ alkyl;

R⁶ is —(CH₂)ₚ-Q-(CH₂)_{q}—,
wherein p and q are independently 0, 1, 2, or 3, and
Q is a bond (absent), —O—, —OCH(OH)—, —CH(OH)O—, —S—, —S(═O)—, —S(═O)₂—, —NR¹¹, —CH(OH)—, —C(═O)—, —C(═O)O—, or —OC(═O)—,
wherein p and q are selected such that:
   2≤(p+q)≤4 if Q is a bond,
   1≤(p+q)≤3 if Q is —O—, S—, —S(═O)—, —S(═O)₂—, —NR¹¹, —CH(OH)—, or —C(═O)—,
   0≤(p+q)≤2 if Q is —C(═O)O—, —OC(═O)—, —OCH(OH)—, or —CH(OH)O—, and
wherein each CH₂ is optionally independently substituted with one or two methyl groups;

R⁷ is selected from the group consisting of H, optionally substituted C₁-C₆ alkyl, and optionally substituted C₃-C₈ cycloalkyl;

each occurrence of R^{8a}, R^{8b}, R^{8c}, R^{8d}, R^{8e}, R^{8f}, R^{8g}, and R^{8h} is independently selected from the group consisting of H, halogen, —CN, optionally substituted C₁-C₆ alkyl, optionally substituted C₃-C₈ cycloalkyl, optionally substituted C₁-C₆ alkoxy, optionally substituted C₃-C₈ cycloalkoxy, heterocyclyl, heteroaryl, —S(optionally substituted C₁-C₆ alkyl), —SO(optionally substituted C₁-C₆ alkyl), —SO₂(optionally substituted C₁-C₆ alkoxy), —C(═O)OH, —C(═O)O(optionally substituted C₁-C₆ alkyl), —C(═O)O(optionally substituted C₃-C₈ cycloalkyl), —O(optionally substituted C₁-C₆ alkyl), —O(optionally substituted C₃-C₈ cycloalkyl), —NH₂, —NH(optionally substituted C₁-C₆ alkyl), —NH(optionally substituted C₃-C₈ cycloalkyl), —N(optionally substituted C₁-C₆ alkyl)(optionally substituted C₁-C₆ alkyl), —N(optionally substituted C₃-C₈ cycloalkyl)(optionally substituted C₃-C₈ cycloalkyl), —N(optionally substituted C₁-C₆ alkyl)(optionally substituted C₃-C₈ cycloalkyl), —C(═O)NH₂, —C(═O)NH(optionally substituted C₁-C₆ alkyl), —C(═O)NH(optionally substituted C₃-C₈ cycloalkyl), —C(═O)N(optionally substituted C₁-C₆ alkyl)(optionally substituted C₁-C₆ alkyl), —C(═O)N(optionally substituted C₃-C₈ cycloalkyl)(optionally substituted C₃-C₈ cycloalkyl), and —C(═O)N(optionally substituted C₁-C₆ alkyl)(optionally substituted C₃-C₈ cycloalkyl;

each occurrence of R⁹ is independently selected from the group consisting of H, optionally substituted C₁-C₆ alkyl, optionally substituted C₃-C₈ cycloalkyl, optionally substituted phenyl, and optionally substituted hetereoaryl;

R¹⁰ is selected from the group consisting of H, halogen, —CN, optionally substituted C₁-C₆ alkyl, optionally substituted C₃-C₈ cycloalkyl, optionally substituted C₁-C₆ alkoxy, optionally substituted C₃-C₈ cycloalkoxy, heterocyclyl, heteroaryl, —S(optionally substituted C₁-C₆ alkyl), —SO(optionally substituted C₁-C₆ alkyl), —SO₂(optionally substituted C₁-C₆ alkyl), —C(═O)OH, —C(═O)O(optionally substituted C₁-C₆ alkyl), —C(═O)O(optionally substituted C₃-C₈ cycloalkyl), —O(optionally substituted C₁-C₆ alkyl), —O(optionally substituted C₃-C₈ cycloalkyl), —NH₂, —NH(optionally substituted C₁-C₆ alkyl), —NH(optionally substituted C₃-C₈ cycloalkyl), —N(optionally substituted C₁-C₆ alkyl)(optionally substituted C₁-C₆ alkyl), —N(optionally substituted C₃-C₈ cycloalkyl)(optionally substituted C₃-C₈ cycloalkyl), —N(optionally substituted C₁-C₆ alkyl)(optionally substituted C₃-C₈ cycloalkyl), —C(═O)NH₂, —C(═O)NH(optionally substituted C₁-C₆ alkyl), —C(═O)NH(optionally substituted C₃-C₈ cycloalkyl), —C(═O)N(optionally substituted C₁-C₆ alkyl)(optionally substituted C₁-C₆ alkyl), —C(═O)N(optionally substituted C₃-C₈ cycloalkyl)(optionally substituted C₃-C₈ cycloalkyl), and —C(═O)N(optionally substituted C₁-C₆ alkyl)(optionally substituted C₃-C₈ cycloalkyl;

R¹¹ is selected from the group consisting of H, optionally substituted C₁-C₆ alkyl, optionally substituted C₃-C₈ cycloalkyl, optionally substituted phenyl, optionally substituted heteroaryl, and optionally substituted C₁-C₆ acyl.

Embodiment 2 provides the compound of Embodiment 1, which is:

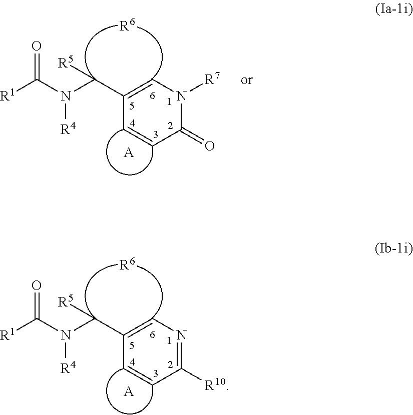

Embodiment 3 provides the compound of any one of Embodiments 1-2, wherein R⁵ is selected from the group consisting of H and CH₃.

Embodiment 4 provides the compound of any one of Embodiments 1-3, wherein each occurrence of aryl or heteroaryl is independently optionally substituted with at least one substituent selected from the group consisting of C₁-C₆ alkyl, C₃-C₈ cycloalkyl, phenyl, C₁-C₆ hydroxyalkyl, (C₁-C₆ alkoxy)-C₁-C₆ alkyl, C₁-C₆ haloalkyl, C₁-C₆ haloalkoxy, halogen, —CN, —OR^{b}, —N(R^{b})(R^{b}), —NO₂, —C(═O)N(R^{b})(R^{b}), —C(═O)OR^{b}, —OC(═O)R^{b}, —SR^{b}, —S(═O)R^{b}, —S(═O)₂R^{b}, N(R^{b})S(═O)₂R^{b}, —S(═O)₂N(R^{b})(R^{b}), acyl, and C₁-C₆ alkoxycarbonyl, wherein each occurrence of R^{b} is independently H, C₁-C₆ alkyl, or C₃-C₈ cycloalkyl, wherein in R^{b} the alkyl or cycloalkyl is optionally substituted with at least one selected from the group consisting of halogen, —OH, C₁-C₆ alkoxy, and heteroaryl; or substituents on two adjacent carbon atoms combine to form —O(CH₂)₁₋₃O—.

Embodiment 5 provides the compound of any one of Embodiments 1-4, wherein each occurrence of alkyl, alkenyl, alkynyl, or cycloalkyl is independently optionally substituted with at least one substituent selected from the group consisting of C₁-C₆ alkyl, C₃-C₈ cycloalkyl, halogen, cyano (—CN), —OR^{a}, optionally substituted phenyl, optionally substituted heteroaryl, optionally substituted heterocyclyl, —C(═O)OR^{a}, —OC(═O)R^{a}, —SR^{a}, —S(═O)R^{a}, —S(═O)₂R^{a}, —S(═O)₂NR^{a}R^{a}, —N(R^{a})S(═O)₂R^{a}, —N(R^{a})C(═O)R^{a}, —C(═O)NR^{a}R^{a}, and —N(R^{a})(R^{a}), wherein each occurrence of R^{a} is independently H, optionally substituted C₁-C₆ alkyl, optionally substituted C₃-C₈ cycloalkyl, optionally substituted aryl, or optionally substituted heteroaryl, or two R^{a} groups combine with the N to which they are bound to form a heterocycle.

Embodiment 6 provides the compound of any one of Embodiments 1-5, wherein R² is phenyl optionally substituted with at least one selected from the group consisting of C₁-C₆ alkyl, halogen, C₁-C₃ haloalkyl, and —CN.

Embodiment 7 provides the compound of any one of Embodiments 1-6, wherein R² is selected from the group consisting of phenyl, 3-chlorophenyl, 4-chlorophenyl, 3-fluorophenyl, 4-fluorophenyl, 3,4-difluorophenyl, 3,5-difluorophenyl, 2,4,5-trifluorophenyl, 3,4,5-trifluorophenyl, 3,4-dichlorophenyl, 3-chloro-4-fluorophenyl, 4-chloro-3-fluorophenyl, 4-chloro-3-methylphenyl, 3-chloro-4-methylphenyl, 4-fluoro-3-methylphenyl, 3-fluoro-4-methylphenyl, 4-chloro-3-methoxyphenyl, 3-chloro-4-methoxyphenyl, 4-fluoro-3-methoxyphenyl, 3-fluoro-4-methoxyphenyl, 3-trifluoromethylphenyl, 4-trifluoromethylphenyl, 3-trifluoromethyl-4-fluorophenyl, 4-trifluoromethyl-3-fluorophenyl, 3-cyanophenyl, 4-cyanophenyl, 3-cyano-4-fluorophenyl, 4-cyano-3-fluorophenyl, 3-difluoromethyl-4-fluorophenyl, and 4-difluoromethyl-3-fluorophenyl.

Embodiment 8 provides the compound of any one of Embodiments 1-7, wherein R³ is selected from the group consisting of H and methyl.

Embodiment 9 provides the compound of any one of Embodiments 1-8, wherein R⁶ is a divalent group selected from the group consisting of —CH₂CH₂—,—CH₂CH₂CH₂—, —CH₂OCH₂—, —CH₂OCH(OH)—, —CH(OH)OCH₂—, —CH₂OC(═O)—, —C(═O)OCH₂—, —CH₂SCH₂—, —CH₂S(═O)CH₂—, —CH₂S(═O)₂CH₂—, —CH₂NHCH₂—, —CH₂N(CH₃)CH₂—, —CH₂N[C(═O)CH₃]CH₂—, —CH₂N[CH₂CH₂OH]CH₂—, —CH₂CH₂CH₂CH₂—, —CH₂OCH₂CH₂—, and —CH₂CH₂OCH₂—, wherein each CH₂ group is optionally independently substituted with one or two CH₃ groups.

Embodiment 10 provides the compound of any one of Embodiments 1-9, which is selected from the group consisting of:

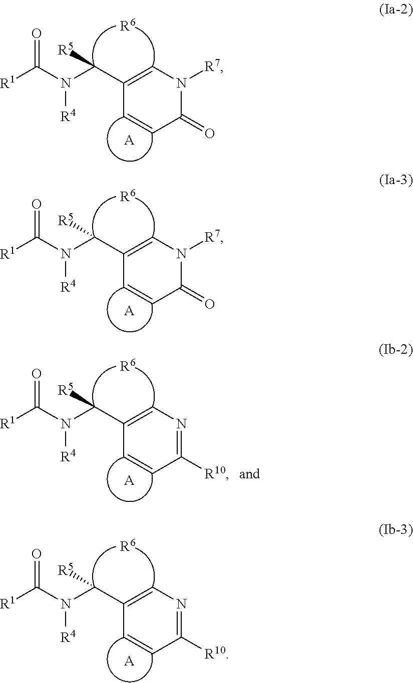

Embodiment 11 provides the compound of any one of Embodiments 1-10, which is selected from the group consisting of:

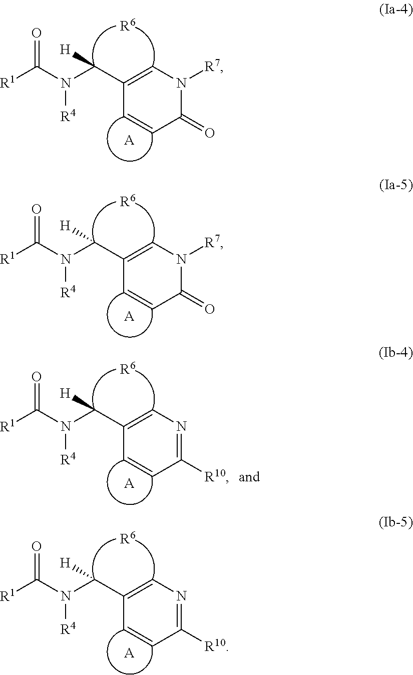

Embodiment 12 provides the compound of any one of Embodiments 1-11, which is selected from the group consisting of:

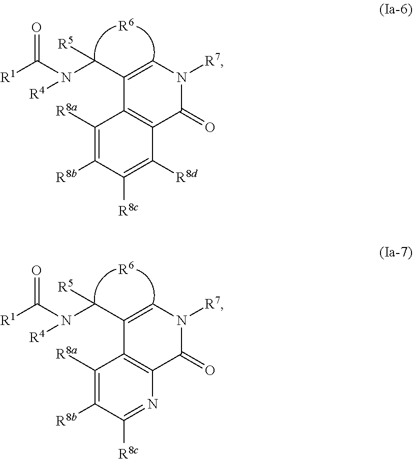

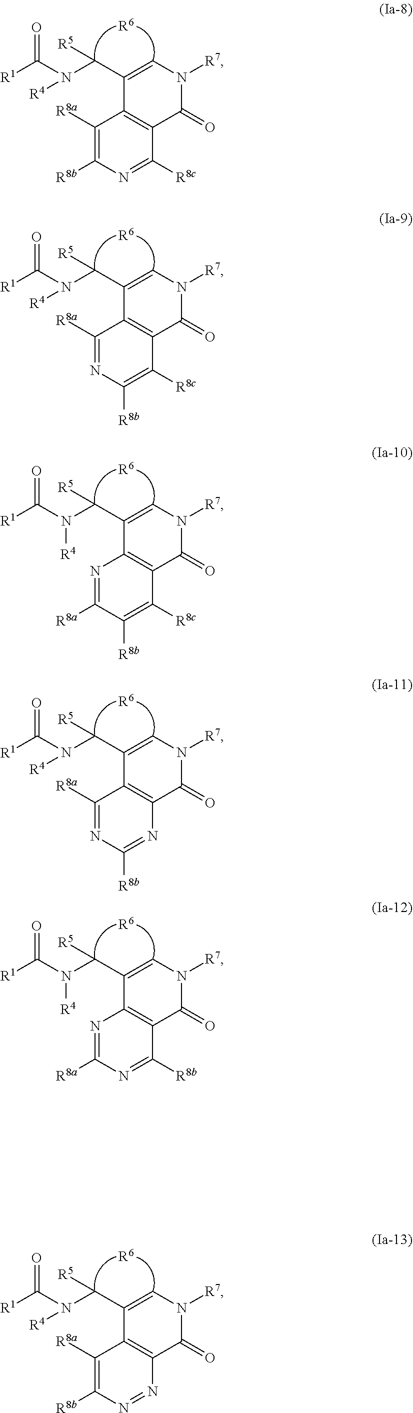

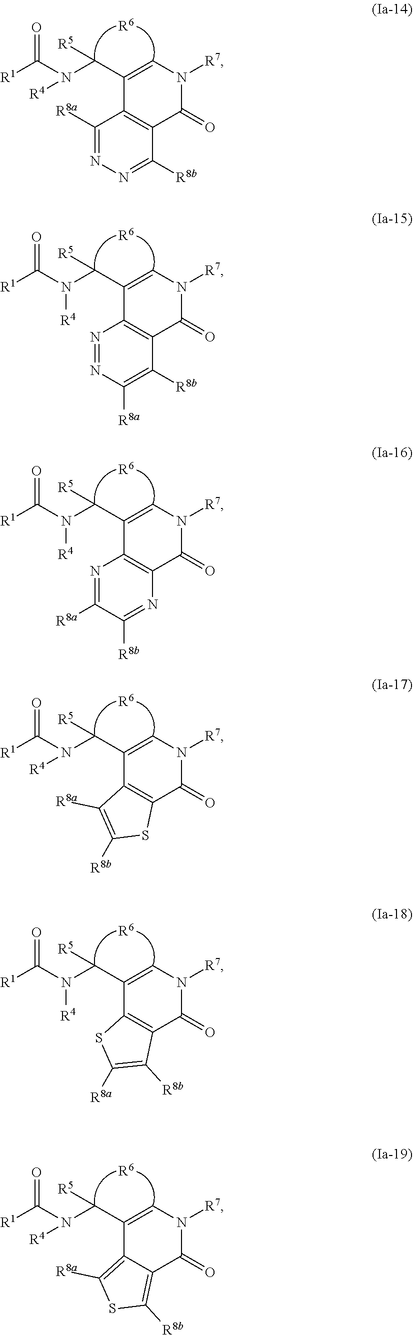

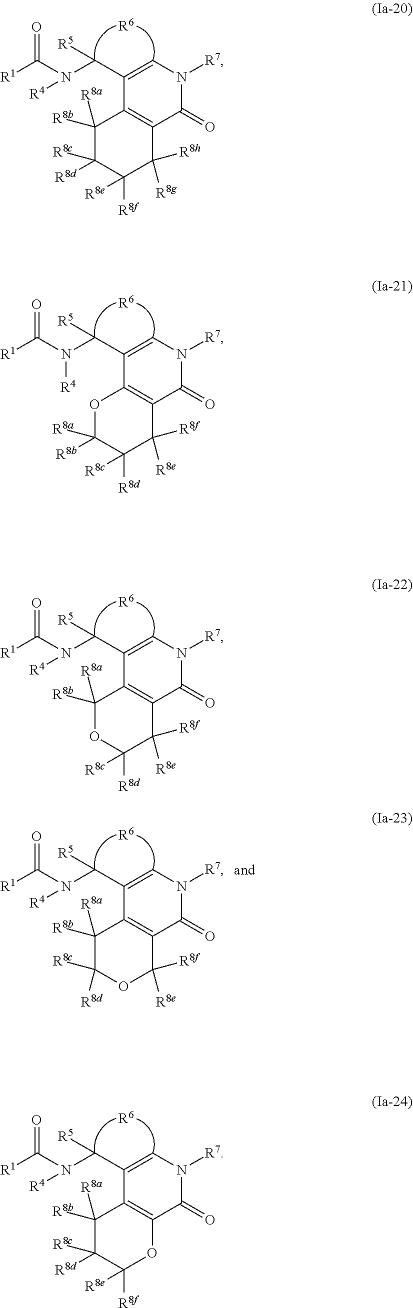

Embodiment 13 provides the compound of any one of Embodiments 1-11, which is selected from the group consisting of:

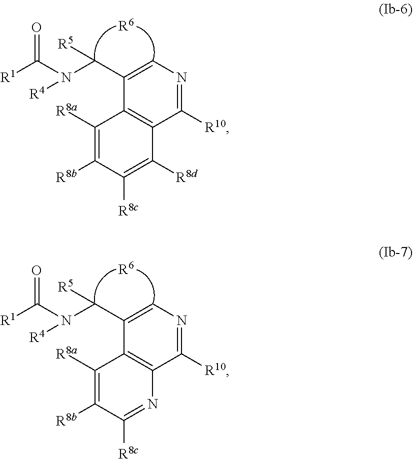

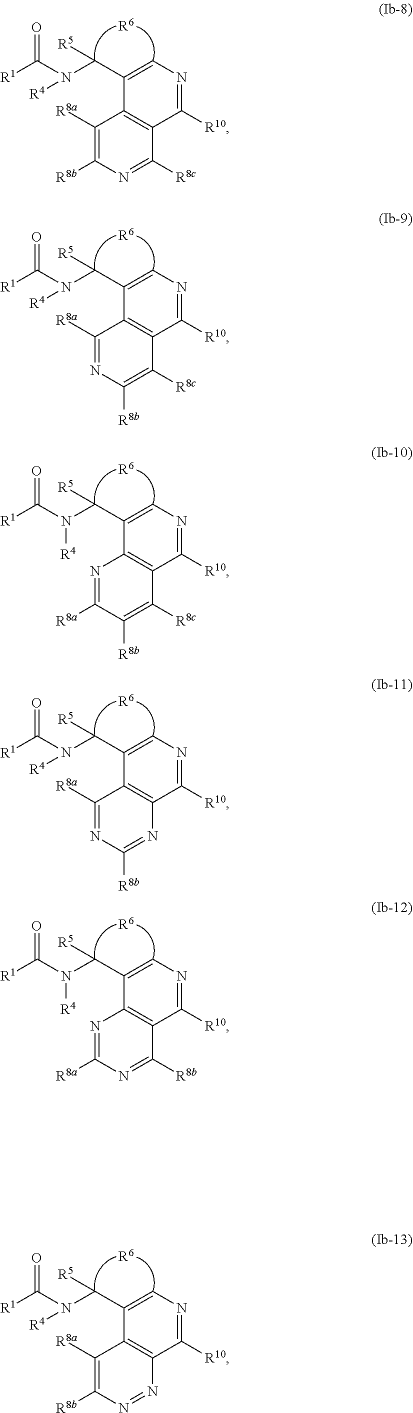

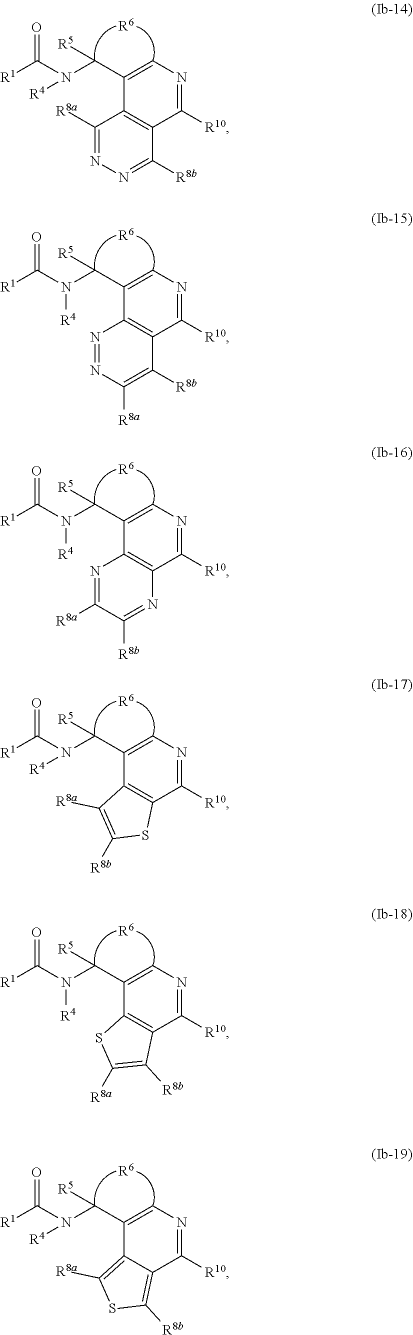

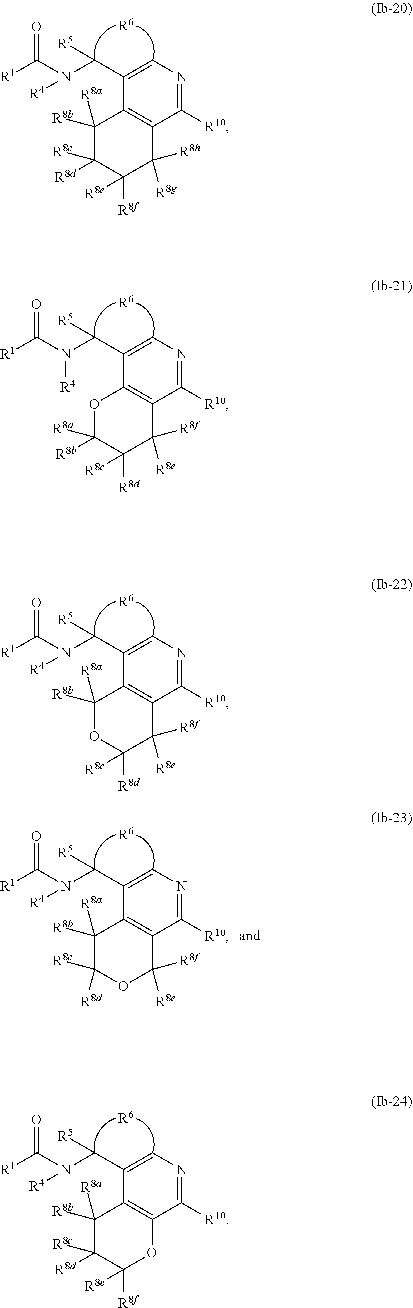

Embodiment 14 provides the compound of any one of Embodiments 1-11, which is selected from the group consisting of:

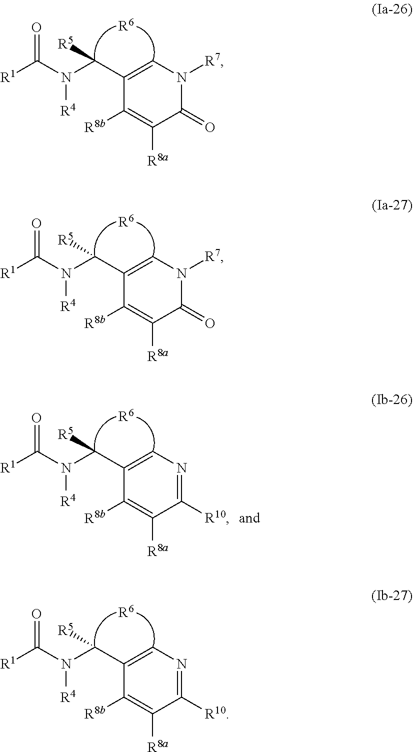

Embodiment 15 provides the compound of any one of Embodiments 1-11, which is selected from the group consisting of:

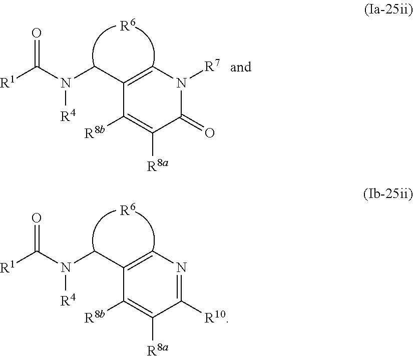

Embodiment 16 provides the compound of any one of Embodiments 1-11 and 14-15, which is at least one selected from the group consisting of:

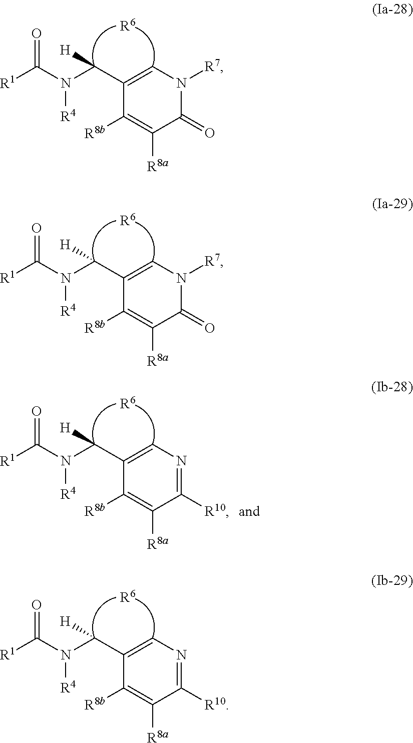

Embodiment 17 provides the compound of any one of Embodiments 1-16, wherein ring B

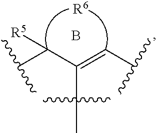

which is formed by R⁶ and the carbon atoms to which R⁶ is attached, is selected from the group consisting of:

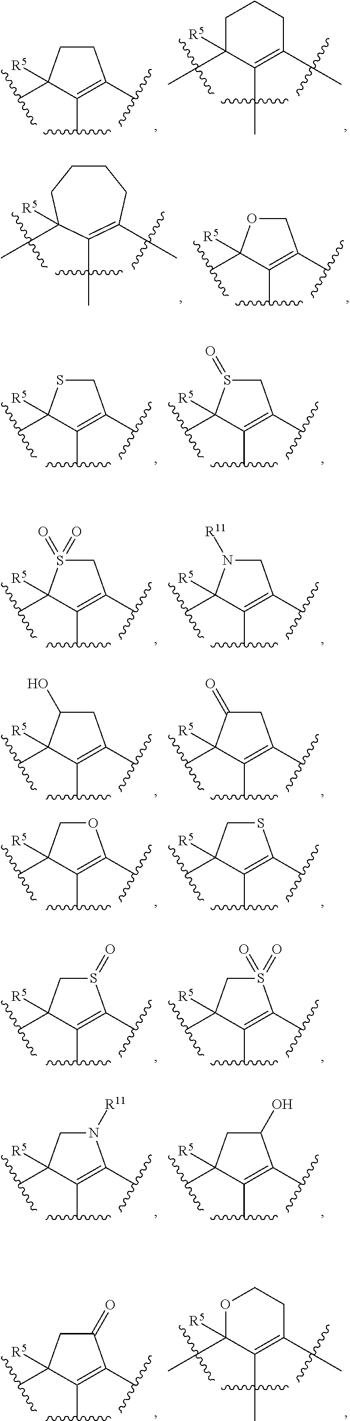

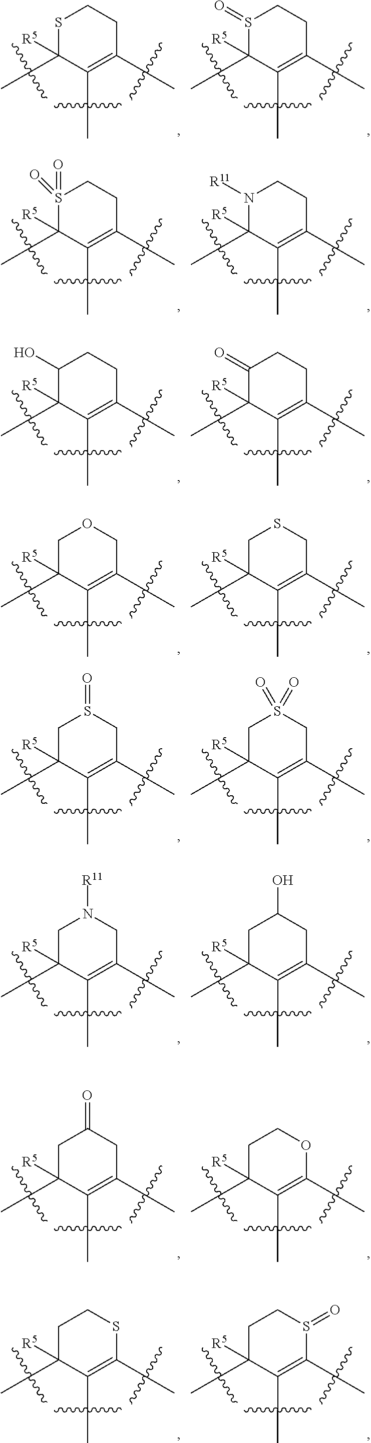

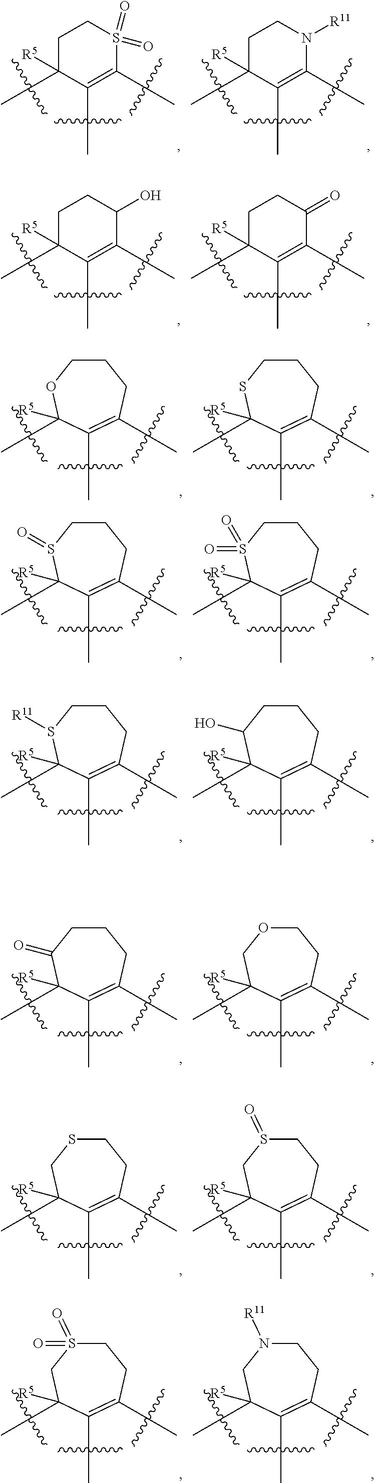

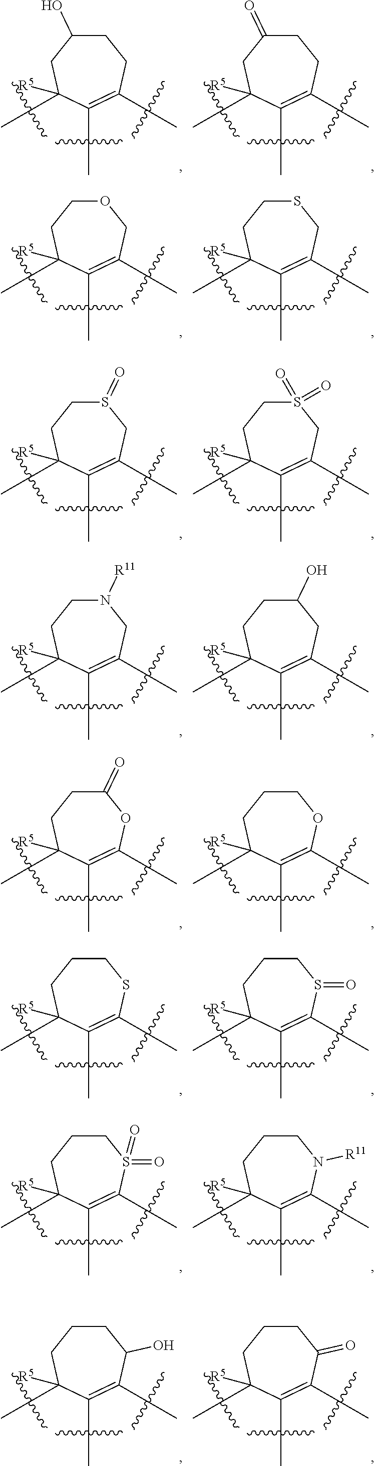

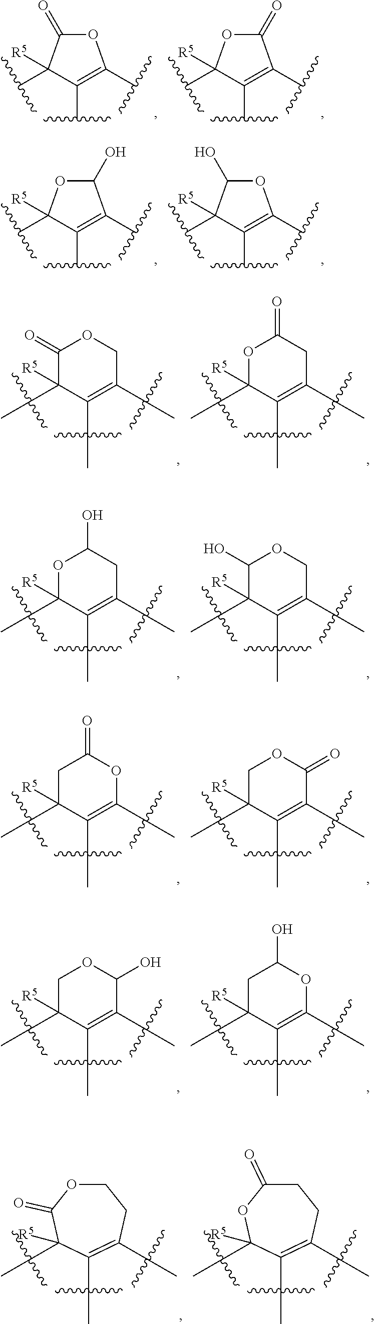

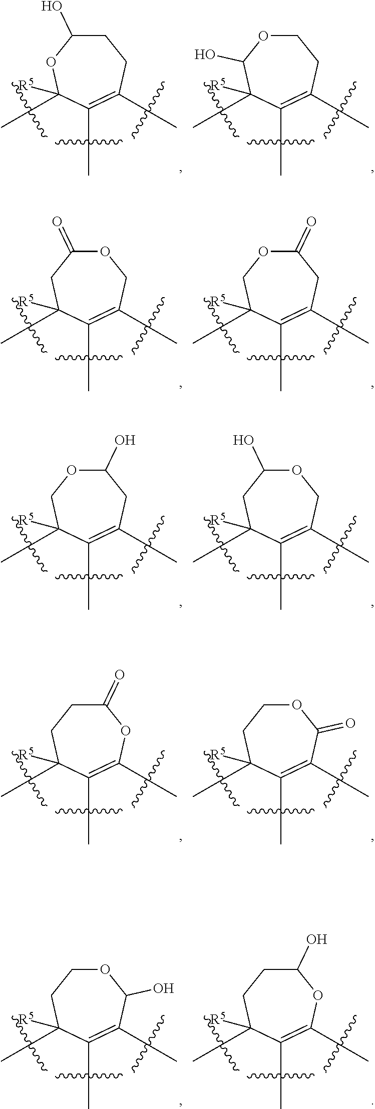

Embodiment 18 provides the compound of any one of Embodiments 1-17, which is at least one selected from the group consisting of:
3-(3-chloro-4-fluorophenyl)-1-isobutyl-1-(2-oxo-4-(trifluoromethyl)-1,2,5,6,7,8-hexahydroquinolin-5-yl)urea;
3-(3-chloro-4-fluorophenyl)-1-methyl-1-(2-oxo-4-(trifluoromethyl)-1,2,5,6,7,8-hexahydroquinolin-5-yl)urea;
3-(3-chloro-4-fluorophenyl)-1-(3-hydroxypropyl)-1-(2-oxo-4-(trifluoromethyl)-1,2,5,6,7,8-hexahydroquinolin-5-yl)urea;
3-(3-chloro-4-fluorophenyl)-1-isobutyl-1-(2-oxo-4-(trifluoromethyl)-2,5,6,7-tetrahydro-1H-cyclopenta[b]pyridin-5-yl)urea;
3-(3,4-difluorophenyl)-1-isobutyl-1-(2-oxo-4-(trifluoromethyl)-2,5,6,7-tetrahydro-1H-cyclopenta[b]pyridin-5-yl)urea;
3-(3-chloro-4-fluorophenyl)-1-methyl-1-(6-oxo-1,2,3,4,5,6-hexahydrophenanthridin-1-yl)urea;
3-(3-chloro-4-fluorophenyl)-1-(3-hydroxypropyl)-1-(6-oxo-1,2,3,4,5,6-hexahydrophenanthridin-1-yl)urea;
3-(3-chloro-4-fluorophenyl)-1-isobutyl-1-(6-oxo-1,2,3,4,5,6-hexahydrophenanthridin-1-yl)urea;
3-(3-chloro-4-fluorophenyl)-1-(8-fluoro-6-oxo-1,2,3,4,5,6-hexahydrophenanthridin-1-yl)-1-methylurea;
3-(3-chloro-4-fluorophenyl)-1-(8-fluoro-6-oxo-1,2,3,4,5,6-hexahydrophenanthridin-1-yl)-1-isobutylurea;
3-(3-chloro-4-fluorophenyl)-1-(8-fluoro-6-oxo-1,2,3,4,5,6-hexahydrophenanthridin-1-yl)-1-(3-hydroxypropyl)urea;
3-(3-chloro-4-fluorophenyl)-1-methyl-1-(5-oxo-2,3,4,5-tetrahydro-1H-cyclopenta[c]isoquinolin-1-yl)urea;
3-(3-chloro-4-fluorophenyl)-1-isobutyl-1-(5-oxo-2,3,4,5-tetrahydro-1H-cyclopenta[c]isoquinolin-1-yl)urea;
3-(3-chloro-4-fluorophenyl)-1-(3-hydroxypropyl)-1-(5-oxo-2,3,4,5-tetrahydro-1H-cyclopenta[c]isoquinolin-1-yl)urea;
3-(3-chloro-4-fluorophenyl)-1-(8,9-difluoro-6-oxo-1,2,3,4,5,6-hexahydrophenanthridin-1-yl)-1-methylurea;
3-(3-chloro-4-fluorophenyl)-1-methyl-1-(6-oxo-1,2,3,4,5,6-hexahydrophenanthridin-1-yl)urea;
3-(3-chloro-4-fluorophenyl)-1-methyl-1-(6-oxo-1,4,5,6-tetrahydro-2H-pyrano[3,4-c]isoquinolin-1-yl)urea;
1-(8,9-difluoro-6-oxo-1,2,3,4,5,6-hexahydrophenanthridin-1-yl)-3-(4-fluorophenyl)-1-methylurea;
3-(3-chloro-4-fluorophenyl)-1-(8,10-difluoro-6-oxo-1,2,3,4,5,6-hexahydrophenanthridin-1-yl)-1-methylurea;
3-(3-chloro-4-fluorophenyl)-1-methyl-1-(5-oxo-6,7,8,9,10,11-hexahydro-5H-cyclohepta[c]isoquinolin-11-yl)urea;
3-(3-chloro-4-fluorophenyl)-1-(3-hydroxypropyl)-1-(5-oxo-6,7,8,9,10,11-hexahydro-5H-cyclohepta[c]isoquinolin-11-yl)urea;
3-(3-chloro-4-fluorophenyl)-1-(8-fluoro-6-oxo-1,4,5,6-tetrahydro-2H-pyrano[3,4-c]isoquinolin-1-yl)-1-methylurea;
3-(3-chloro-4-fluorophenyl)-1-(8,9-difluoro-6-oxo-1,4,5,6-tetrahydro-2H-pyrano[3,4-c]isoquinolin-1-yl)-1-methylurea;
3-(3-chloro-4-fluorophenyl)-1-(8,10-difluoro-6-oxo-1,4,5,6-tetrahydro-2H-pyrano[3,4-c]isoquinolin-1-yl)-1-methylurea;
3-(3-chloro-4-fluorophenyl)-1-methyl-1-(3-methyl-6-oxo-1,2,3,4,5,6-hexahydrophenanthridin-1-yl)urea;
3-(3-chloro-4-fluorophenyl)-1-(3,3-dimethyl-6-oxo-1,2,3,4,5,6-hexahydrophenanthridin-1-yl)-1-methylurea;
3-(3-chloro-4-fluorophenyl)-1-(7,8-difluoro-6-oxo-1,2,3,4,5,6-hexahydrophenanthridin-1-yl)-1-methylurea;
3-(3-chloro-5-fluorophenyl)-1-methyl-1-(6-oxo-1,4,5,6-tetrahydro-2H-pyrano[3,4-c]isoquinolin-1-yl)urea;
3-(3-chloro-4-fluorophenyl)-1-isobutyl-1-(6-oxo-1,4,5,6-tetrahydro-2H-pyrano[3,4-c]isoquinolin-1-yl)urea;
3-(3-chloro-4-fluorophenyl)-1-methyl-1-(5-methyl-6-oxo-1,2,3,4,5,6-hexahydrophenanthridin-1-yl)urea;
3-(3,4-difluorophenyl)-1-(8-fluoro-6-oxo-1,2,3,4,5,6-hexahydrophenanthridin-1-yl)-1-methylurea;
1-(8-fluoro-6-oxo-1,2,3,4,5,6-hexahydrophenanthridin-1-yl)-1-methyl-3-(3,4,5-trifluorophenyl)urea;
3-(3-chloro-4-fluorophenyl)-1-(6-methoxy-1,2,3,4-tetrahydrophenanthridin-1-yl)-1-methylurea;
3-(3-chloro-4-fluorophenyl)-1-(7,8-difluoro-6-oxo-1,4,5,6-tetrahydro-2H-pyrano[3,4-c]isoquinolin-1-yl)-1-methylurea;
3-(3-chloro-4-fluorophenyl)-1-(8-fluoro-6-oxo-1,4,5,6-tetrahydro-2H-pyrano[3,4-c]isoquinolin-1-yl)-1-isobutylurea;
3-(3-chloro-4-fluorophenyl)-1-ethyl-1-(8-fluoro-6-oxo-1,4,5,6-tetrahydro-2H-pyrano[3,4-c]isoquinolin-1-yl)urea;
3-(3-chloro-4-fluorophenyl)-1-(8,9-difluoro-6-oxo-1,4,5,6-tetrahydro-2H-pyrano[3,4-c]isoquinolin-1-yl)-1-ethylurea;
3-(3-chloro-4-fluorophenyl)-1-methyl-1-(6-oxo-1,2,3,4,5,6,7,8,9,10-decahydrophenanthridin-1-yl)urea;
3-(3,4-difluorophenyl)-1-methyl-1-(6-oxo-1,2,3,4,5,6,7,8,9,10-decahydrophenanthridin-1-yl)urea;
3-(3,4-difluorophenyl)-1-ethyl-1-(6-oxo-1,2,3,4,5,6,7,8,9,10-decahydrophenanthridin-1-yl)urea;
3-(3-chloro-4-fluorophenyl)-1-ethyl-1-(6-oxo-1,2,3,4,5,6,7,8,9,10-decahydrophenanthridin-1-yl)urea;
3-(3-chloro-4-fluorophenyl)-1-(8-chloro-6-oxo-1,4,5,6-tetrahydro-2H-pyrano[3,4-c]isoquinolin-1-yl)-1-methylurea;
3-(3-chloro-4-fluorophenyl)-1-(8-chloro-6-oxo-1,4,5,6-tetrahydro-2H-pyrano[3,4-c]isoquinolin-1-yl)-1-ethylurea;
3-(4-fluoro-3-methylphenyl)-1-(8-fluoro-6-oxo-1,4,5,6-tetrahydro-2H-pyrano[3,4-c]isoquinolin-1-yl)-1-methylurea;
1-(8-chloro-6-oxo-1,4,5,6-tetrahydro-2H-pyrano[3,4-c]isoquinolin-1-yl)-3-(4-fluoro-3-methylphenyl)-1-methylurea;
1-(8-chloro-6-oxo-1,4,5,6-tetrahydro-2H-pyrano[3,4-c]isoquinolin-1-yl)-1-ethyl-3-(4-fluoro-3-methylphenyl)urea;
3-(3-chloro-4-fluorophenyl)-1-methyl-1-(5-oxo-1,4,5,6,7,8,9,10-octahydro-2H-pyrano[3,4-c]quinolin-10-yl)urea;
3-(3,4-difluorophenyl)-1-methyl-1-(5-oxo-1,4,5,6,7,8,9,10-octahydro-2H-pyrano[3,4-c]quinolin-10-yl)urea;
3-(3-cyano-4-fluorophenyl)-1-(8-fluoro-6-oxo-1,4,5,6-tetrahydro-2H-pyrano[3,4-c]isoquinolin-1-yl)-1-methylurea;
1-(8-chloro-6-oxo-1,4,5,6-tetrahydro-2H-pyrano[3,4-c]isoquinolin-1-yl)-3-(3-cyano-4-fluorophenyl)-1-methylurea;
1-(8-chloro-6-oxo-1,4,5,6-tetrahydro-2H-pyrano[3,4-c]isoquinolin-1-yl)-3-(3-cyano-4-fluorophenyl)-1-ethylurea;
1-(3-chloro-4-fluorophenyl)-3-(8-fluoro-6-oxo-1,4,5,6-tetrahydro-2H-pyrano[3,4-c]isoquinolin-1-yl)urea;
1-(3-chloro-4-fluorophenyl)-3-(8,9-difluoro-6-oxo-1,4,5,6-tetrahydro-2H-pyrano[3,4-c]isoquinolin-1-yl)urea;
1-(8,9-difluoro-6-oxo-1,4,5,6-tetrahydro-2H-pyrano[3,4-c]isoquinolin-1-yl)-3-(4-fluoro-3-methylphenyl)-1-methylurea;
3-(3-chloro-4-fluorophenyl)-1-(9-fluoro-6-oxo-1,4,5,6-tetrahydro-2H-pyrano[3,4-c]isoquinolin-1-yl)-1-methylurea;
3-(3-chloro-4-fluorophenyl)-1-ethyl-1-(9-fluoro-6-oxo-1,4,5,6-tetrahydro-2H-pyrano[3,4-c]isoquinolin-1-yl)urea;
3-(4-fluoro-3-methylphenyl)-1-(9-fluoro-6-oxo-1,4,5,6-tetrahydro-2H-pyrano[3,4-c]isoquinolin-1-yl)-1-methylurea;
1-ethyl-3-(4-fluoro-3-methylphenyl)-1-(9-fluoro-6-oxo-1,4,5,6-tetrahydro-2H-pyrano[3,4-c]isoquinolin-1-yl)urea;
3-(3-cyano-4-fluorophenyl)-1-(9-fluoro-6-oxo-1,4,5,6-tetrahydro-2H-pyrano[3,4-c]isoquinolin-1-yl)-1-methylurea;
3-(3-cyano-4-fluorophenyl)-1-ethyl-1-(9-fluoro-6-oxo-1,4,5,6-tetrahydro-2H-pyrano[3,4-c]isoquinolin-1-yl)urea;
3-(3-chloro-4-fluorophenyl)-1-methyl-1-(5-oxo-3,4,5,6,7,8,9,10-octahydro-1H-pyrano[4,3-c]quinolin-10-yl)urea;
3-(3,4-difluorophenyl)-1-methyl-1-(5-oxo-3,4,5,6,7,8,9,10-octahydro-1H-pyrano[4,3-c]quinolin-10-yl)urea;
3-(3-chloro-4-fluorophenyl)-1-methyl-1-(5-oxo-4,5,6,7,9,10-hexahydro-1H,3H-dipyrano[3,4-b:3′,4′-d]pyridin-10-yl)urea;
3-(3,4-difluorophenyl)-1-methyl-1-(5-oxo-4,5,6,7,9,10-hexahydro-1H,3H-dipyrano[3,4-b:3′,4′-d]pyridin-10-yl)urea;
3-(3-chloro-4-fluorophenyl)-1-(8-cyano-6-oxo-1,4,5,6-tetrahydro-2H-pyrano[3,4-c]isoquinolin-1-yl)-1-methylurea;
3-(3-chloro-4-fluorophenyl)-1-(8-cyano-6-oxo-1,4,5,6-tetrahydro-2H-pyrano[3,4-c]isoquinolin-1-yl)-1-ethylurea;
3-(3-chloro-4-fluorophenyl)-1-methyl-1-(6-oxo-1,2,4,5,6,7,9,10-octahydrodipyrano[3,4-b:4′,3′-d]pyridin-1-yl)urea;
3-(3,4-difluorophenyl)-1-methyl-1-(6-oxo-1,2,4,5,6,7,9,10-octahydrodipyrano[3,4-b:4′,3′-d]pyridin-1-yl)urea;
3-(3-chloro-4-fluorophenyl)-1-methyl-1-(6-oxo-1,4,5,6-tetrahydro-2H-pyrano[3,4-b]thieno[3,2-d]pyridin-1-yl)urea;
3-(3-chloro-4-fluorophenyl)-1-methyl-1-(4-oxo-4,6,8,9-tetrahydro-5H-pyrano[3,4-b]thieno[2,3-d]pyridin-9-yl)urea;
3-(3,5-dichloro-4-fluorophenyl)-1-(8-fluoro-6-oxo-1,4,5,6-tetrahydro-2H-pyrano[3,4-c]isoquinolin-1-yl)-1-methylurea;
3-(3,4-difluorophenyl)-1-(8-fluoro-6-oxo-1,4,5,6-tetrahydro-2H-pyrano[3,4-c]isoquinolin-1-yl)-1-isobutylurea;
1-(8,9-difluoro-6-oxo-1,4,5,6-tetrahydro-2H-pyrano[3,4-c]isoquinolin-1-yl)-1-methyl-3-phenylurea;
1-(8,9-difluoro-6-oxo-1,4,5,6-tetrahydro-2H-pyrano[3,4-c]isoquinolin-1-yl)-3-(4-fluorophenyl)-1-methylurea;
3-(3-chloro-4-fluorophenyl)-1-methyl-1-(4-oxo-4,5,8,9-tetrahydro-6H-pyrano[3,4-b]thieno[3,4-d]pyridin-9-yl)urea;
3-(3-cyano-4-fluorophenyl)-1-(8,9-difluoro-6-oxo-1,4,5,6-tetrahydro-2H-pyrano[3,4-c]isoquinolin-1-yl)-1-methylurea;
3-(3-chloro-4,5-difluorophenyl)-1-(8-fluoro-6-oxo-1,4,5,6-tetrahydro-2H-pyrano[3,4-c]isoquinolin-1-yl)-1-methylurea;
3-(3-chloro-4-fluorophenyl)-1-(8-fluoro-6-oxo-1,4,5,6-tetrahydro-2H-pyrano[3,4-c]isoquinolin-1-yl)-1-(3-hydroxypropyl)urea;
3-(3-chloro-4-fluorophenyl)-1-(8-fluoro-6-oxo-1,4,5,6-tetrahydro-2H-pyrano[3,4-c]isoquinolin-1-yl)-1-(2-hydroxy-2-methylpropyl)urea;
1-(8-fluoro-6-oxo-1,4,5,6-tetrahydro-2H-pyrano[3,4-c]isoquinolin-1-yl)-1-isobutyl-3-(3,4,5-trifluorophenyl)urea;
3-(3-cyano-4-fluorophenyl)-1-(8-fluoro-6-oxo-1,4,5,6-tetrahydro-2H-pyrano[3,4-c]isoquinolin-1-yl)-1-isobutylurea;
1-(8,9-difluoro-6-oxo-1,4,5,6-tetrahydro-2H-pyrano[3,4-c]isoquinolin-1-yl)-3-(3,4-difluorophenyl)-1-methylurea;
3-(3-chlorophenyl)-1-(8,9-difluoro-6-oxo-1,4,5,6-tetrahydro-2H-pyrano[3,4-c]isoquinolin-1-yl)-1-methylurea;
3-(3-cyano-4-fluorophenyl)-1-(8,9-difluoro-6-oxo-1,4,5,6-tetrahydro-2H-pyrano[3,4-c]isoquinolin-1-yl)-1-isobutylurea;
3-(3-(difluoromethyl)-4-fluorophenyl)-1-(8-fluoro-6-oxo-1,4,5,6-tetrahydro-2H-pyrano[3,4-c]isoquinolin-1-yl)-1-methylurea;
1-(8,9-difluoro-6-oxo-1,4,5,6-tetrahydro-2H-pyrano[3,4-c]isoquinolin-1-yl)-3-(3-(difluoromethyl)-4-fluorophenyl)-1-methylurea;
3-(4-fluoro-3-methylphenyl)-1-(8-fluoro-6-oxo-1,4,5,6-tetrahydro-2H-pyrano[3,4-c]isoquinolin-1-yl)-1-isobutylurea;
1-(8,9-difluoro-6-oxo-1,4,5,6-tetrahydro-2H-pyrano[3,4-c]isoquinolin-1-yl)-1-methyl-3-(3,4,5-trifluorophenyl)urea;
1-(3-cyano-4-fluorophenyl)-3-(8,9-difluoro-6-oxo-1,4,5,6-tetrahydro-2H-pyrano[3,4-c]isoquinolin-1-yl)urea;
2-(3-(3-chloro-4-fluorophenyl)-1-(8,9-difluoro-6-oxo-1,4,5,6-tetrahydro-2H-pyrano[3,4-c]isoquinolin-1-yl)ureido)ethane-1-sulfonamide;
3-(3-cyano-4-fluorophenyl)-1-(8,9-difluoro-6-oxo-1,4,5,6-tetrahydro-2H-pyrano[3,4-c]isoquinolin-1-yl)-1-ethylurea;
3-(3-chloro-4-fluorophenyl)-1-(8,9-difluoro-6-oxo-1,4,5,6-tetrahydro-2H-pyrano[3,4-c]isoquinolin-1-yl)-1-(2-(methylsulfonyl)ethyl)urea;
3-(4-chloro-3-fluorophenyl)-1-(8,9-difluoro-6-oxo-1,4,5,6-tetrahydro-2H-pyrano[3,4-c]isoquinolin-1-yl)-1-methylurea;
3-(4-chloro-3-cyanophenyl)-1-(8,9-difluoro-6-oxo-1,4,5,6-tetrahydro-2H-pyrano[3,4-c]isoquinolin-1-yl)-1-methylurea;
3-(3,4-dichlorophenyl)-1-(8,9-difluoro-6-oxo-1,4,5,6-tetrahydro-2H-pyrano[3,4-c]isoquinolin-1-yl)-1-methylurea;
1-(8,9-difluoro-6-oxo-1,2,3,4,5,6-hexahydrophenanthridin-1-yl)-1-methyl-3-(1-(trifluoromethyl)cyclopropyl)urea;
1-(8,9-difluoro-6-oxo-1,4,5,6-tetrahydro-2H-pyrano[3,4-c]isoquinolin-1-yl)-1-methyl-3-(1-(trifluoromethyl)cyclopropyl)urea;
3-(3-Chloro-4-fluorophenyl)-1-(8,9-difluoro-6-oxo-1,4,5,6-tetrahydro-2H-pyrano[3,4-c]isoquinolin-1-yl-1-d)-1-(methyl-d3)urea;
3-(3-Chloro-4-methoxyphenyl)-1-(8,9-difluoro-6-oxo-1,4,5,6-tetrahydro-2H-pyrano[3,4-c]isoquinolin-1-yl)-1-methylurea;
3-(3-Chloro-4-hydroxyphenyl)-1-(8,9-difluoro-6-oxo-1,4,5,6-tetrahydro-2H-pyrano[3,4-c]isoquinolin-1-yl)-1-methylurea;
3-(3-Chloro-4-fluorophenyl)-1-(8,9-difluoro-6-oxo-1,2,3,4,5,6-hexahydrobenzo[c][1,7]naphthyridin-1-yl)-1-methylurea;
3-(3-Chloro-4-fluorophenyl)-1-(8,9-difluoro-4-hydroxy-6-oxo-1,4,5,6-tetrahydro-2H-pyrano[3,4-c]isoquinolin-1-yl)-1-methylurea;
3-(3-chloro-4-Fluorophenyl)-1-(8,9-difluoro-4,6-dioxo-1,4,5,6-tetrahydro-2H-pyrano[3,4-c]isoquinolin-1-yl)-1-methylurea;
3-(3-Chloro-4-fluorophenyl)-1-(8-fluoro-6-oxo-1,2,3,4,5,6-hexahydrobenzo[c][1,7]naphthyridin-1-yl)-1-methylurea;
3-(3-Chloro-4-fluorophenyl)-1-(8-fluoro-3-methyl-6-oxo-1,2,3,4,5,6-hexahydrobenzo[c][1,7]naphthyridin-1-yl)-1-methylurea;
1-(3-Acetyl-8-fluoro-6-oxo-1,2,3,4,5,6-hexahydrobenzo[c][1,7]naphthyridin-1-yl)-3-(3-chloro-4-fluorophenyl)-1-methylurea;
1-(3-Acetyl-8,9-difluoro-6-oxo-1,2,3,4,5,6-hexahydrobenzo[c][1,7]naphthyridin-1-yl)-3-(3-chloro-4-fluorophenyl)-1-methylurea;
3-(3-Cyano-4-fluorophenyl)-1-(8-fluoro-6-oxo-1,2,3,4,5,6-hexahydrobenzo[c][1,7]naphthyridin-1-yl)-1-methylurea;
3-(3-Cyano-4-fluorophenyl)-1-(8-fluoro-3-methyl-6-oxo-1,2,3,4,5,6-hexahydrobenzo[c][1,7]naphthyridin-1-yl)-1-methylurea;
1-(3-Acetyl-8-fluoro-6-oxo-1,2,3,4,5,6-hexahydrobenzo[c][1,7]naphthyridin-1-yl)-3-(3-cyano-4-fluorophenyl)-1-methylurea;
3-(3-Chloro-4-fluorophenyl)-1-(8,9-difluoro-3-methyl-6-oxo-1,2,3,4,5,6-hexahydrobenzo[c][1,7]naphthyridin-1-yl)-1-methylurea;
3-(3-Chloro-4-fluorophenyl)-1-(8-fluoro-3-(2-hydroxyethyl)-6-oxo-1,2,3,4,5,6-hexahydrobenzo[c][1,7]naphthyridin-1-yl)-1-methylurea;
3-(3-Cyano-4-fluorophenyl)-1-(8-fluoro-3-(2-hydroxyethyl)-6-oxo-1,2,3,4,5,6-hexahydrobenzo[c][1,7]naphthyridin-1-yl)-1-methylurea;
3-(3-Cyano-4-fluorophenyl)-1-(8,9-difluoro-3-methyl-6-oxo-1,2,3,4,5,6-hexahydrobenzo[c][1,7]naphthyridin-1-yl)-1-methylurea;
3-(3-Cyano-4-fluorophenyl)-1-(8,9-difluoro-6-oxo-1,2,3,4,5,6-hexahydrobenzo[c][1,7]naphthyridin-1-yl)-1-methylurea;
1-(3-Acetyl-8,9-difluoro-6-oxo-1,2,3,4,5,6-hexahydrobenzo[c][1,7]naphthyridin-1-yl)-3-(3-cyano-4-fluorophenyl)-1-methylurea;
3-(3-Chloro-4-fluorophenyl)-1-(8,9-difluoro-3-(2-hydroxyethyl)-6-oxo-1,2,3,4,5,6-hexahydrobenzo[c][1,7]naphthyridin-1-yl)-1-methylurea;
3-(3-Cyano-4-fluorophenyl)-1-(8,9-difluoro-3-(2-hydroxyethyl)-6-oxo-1,2,3,4,5,6-hexahydrobenzo[c][1,7]naphthyridine-1-yl)-1-methylurea;
3-(3-Chloro-4-fluorophenyl)-1-(8-fluoro-6-oxo-1,4,5,6-tetrahydro-2H-thiopyrano[3,4-c]isoquinolin-1-yl)-1-methylurea;
3-(3-Cyano-4-fluorophenyl)-1-(8-fluoro-6-oxo-1,4,5,6-tetrahydro-2H-thiopyrano[3,4-c]isoquinolin-1-yl)-1-methylurea;
3-(3-Chloro-4-fluorophenyl)-1-(8,9-difluoro-6-oxo-1,4,5,6-tetrahydro-2H-thiopyrano[3,4-c]isoquinolin-1-yl)-1-methylurea;
3-(3-Cyano-4-fluorophenyl)-1-(8,9-difluoro-6-oxo-1,4,5,6-tetrahydro-2H-thiopyrano[3,4-c]isoquinolin-1-yl)-1-methylurea;
1-(8,9-Difluoro-6-oxo-1,2,3,4,5,6-hexahydrobenzo[c][1,7]naphthyridin-1-yl)-3-(3,4-difluorophenyl)-1-methylurea;
3-(3-Chloro-4-fluorophenyl)-1-(8-fluoro-3-oxido-6-oxo-1,4,5,6-tetrahydro-2H-thiopyrano[3,4-c]isoquinolin-1-yl)-1-methylurea;
3-(3-Chloro-4-fluorophenyl)-1-(8-fluoro-3,3-dioxido-6-oxo-1,4,5,6-tetrahydro-2H-thiopyrano[3,4-c]isoquinolin-1-yl)-1-methylurea;
3-(3-Cyano-4-fluorophenyl)-1-(8-fluoro-3-oxido-6-oxo-1,4,5,6-tetrahydro-2H-thiopyrano[3,4-c]isoquinolin-1-yl)-1-methylurea;
3-(3-Chloro-4-fluorophenyl)-1-(8,9-difluoro-3,3-dioxido-6-oxo-1,4,5,6-tetrahydro-2H-thiopyrano[3,4-c]isoquinolin-1-yl)-1-methylurea;
3-(3-Cyano-4-fluorophenyl)-1-(8,9-difluoro-3,3-dioxido-6-oxo-1,4,5,6-tetrahydro-2H-thiopyrano[3,4-c]isoquinolin-1-yl)-1-methylurea;
3-(3-Chloro-4-fluorophenyl)-1-(8,9-difluoro-3-oxido-6-oxo-1,4,5,6-tetrahydro-2H-thiopyrano[3,4-c]isoquinolin-1-yl)-1-methylurea;
3-(3-Cyano-4-fluorophenyl)-1-(8,9-difluoro-3-oxido-6-oxo-1,4,5,6-tetrahydro-2H-thiopyrano[3,4-c]isoquinolin-1-yl)-1-methylurea;
3-(3-Cyano-4-fluorophenyl)-1-(8-fluoro-3,3-dioxido-6-oxo-1,4,5,6-tetrahydro-2H-thiopyrano[3,4-c]isoquinolin-1-yl)-1-methylurea;
1-(8,9-difluoro-6-oxo-1,2,3,4,5,6-hexahydrobenzo[c][1,7]naphthyridin-1-yl)-3-(3-(difluoromethyl)-4-fluorophenyl)-1-methylurea;
N-(8,9-difluoro-6-oxo-1,4,5,6-tetrahydro-2H-pyrano[3,4-c]isoquinolin-1-yl)-N-methylisoindoline-2-carboxamide;
5-chloro-N-(8,9-difluoro-6-oxo-1,4,5,6-tetrahydro-2H-pyrano[3,4-c]isoquinolin-1-yl)-N-methylisoindoline-2-carboxamide;
5-bromo-N-(8,9-difluoro-6-oxo-1,4,5,6-tetrahydro-2H-pyrano[3,4-c]isoquinolin-1-yl)-N-methylisoindoline-2-carboxamide;
5-fluoro-N-(8,9-difluoro-6-oxo-1,4,5,6-tetrahydro-2H-pyrano[3,4-c]isoquinolin-1-yl)-N-methylisoindoline-2-carboxamide;
N-(8,9-difluoro-6-oxo-1,2,3,4,5,6-hexahydrobenzo[c][1,7]naphthyridin-1-yl)-N-methylisoindoline-2-carboxamide;
N-(8,9-difluoro-6-oxo-1,2,3,4,5,6-hexahydrobenzo[c][1,7]naphthyridin-1-yl)-5-fluoro-N-methylisoindoline-2-carboxamide;
N-(8,9-difluoro-6-oxo-1,2,3,4,5,6-hexahydrobenzo[c][1,7]naphthyridin-1-yl)-5-chloro-N-methylisoindoline-2-carboxamide;
N-(8,9-difluoro-6-oxo-1,2,3,4,5,6-hexahydrobenzo[c][1,7]naphthyridin-1-yl)-5-bromo-N-methylisoindoline-2-carboxamide;
N-(8,9-difluoro-6-oxo-1,4,5,6-tetrahydro-2H-pyrano[3,4-c]isoquinolin-1-yl)-N-methyl-5-(trifluoromethyl)isoindoline-2-carboxamide;
N-(8,9-difluoro-6-oxo-1,2,3,4,5,6-hexahydrobenzo[c][1,7]naphthyridin-1-yl)-N-methyl-5-(trifluoromethyl)isoindoline-2-carboxamide;
1-(8,9-difluoro-5-methyl-6-oxo-1,4,5,6-tetrahydro-2H-pyrano[3,4-c]isoquinolin-1-yl)-3-(3-(difluoromethyl)-4-fluorophenyl)-1-methylurea;
1-(8,9-difluoro-6-methoxy-1,4-dihydro-2H-pyrano[3,4-c]isoquinolin-1-yl)-3-(3-(difluoromethyl)-4-fluorophenyl)-1-methylurea;
1-(8,9-difluoro-6-(methylamino)-1,4-dihydro-2H-pyrano[3,4-c]isoquinolin-1-yl)-3-(3-(difluoromethyl)-4-fluorophenyl)-1-methylurea
1-(8,9-difluoro-6-((2-hydroxyethyl)amino)-1,4-dihydro-2H-pyrano[3,4-c]isoquinolin-1-yl)-3-(3-(difluoromethyl)-4-fluorophenyl)-1-methylurea;
1-(8,9-difluoro-6-((2-aminoethyl)amino)-1,4-dihydro-2H-pyrano[3,4-c]isoquinolin-1-yl)-3-(3-(difluoromethyl)-4-fluorophenyl)-1-methylurea
3-(3-chloro-4-fluorophenyl)-1-(8,9-difluoro-6-((2-aminoethyl)amino)-1,4-dihydro-2H-pyrano[3,4-c]isoquinolin-1-yl)-1-methylurea;
3-(3-chloro-4-fluorophenyl)-1-(8,9-difluoro-6-(methylamino)-1,4-dihydro-2H-pyrano[3,4-c]isoquinolin-1-yl)-1-methylurea;
1-(8,9-difluoro-5-methyl-6-oxo-1,4,5,6-tetrahydro-2H-pyrano[3,4-c]isoquinolin-1-yl)-3-(3-chloro-4-fluorophenyl)-1-methylurea;
3-(3-chloro-4-fluorophenyl)-1-(8,9-difluoro-6-methoxy-1,4-dihydro-2H-pyrano[3,4-c]isoquinolin-1-yl)-1-methylurea;
3-(3-chloro-4-fluorophenyl)-1-(8,9-difluoro-6-((2-hydroxyethyl)amino)-1,4-dihydro-2H-pyrano[3,4-c]isoquinolin-1-yl)-1-methylurea;
1-(8,9-difluoro-5-(2-hydroxyethyl)-6-oxo-1,4,5,6-tetrahydro-2H-pyrano[3,4-c]isoquinolin-1-yl)-3-(3-(difluoromethyl)-4-fluorophenyl)-1-methylurea;
1-(6-(2-aminoethoxy)-8,9-difluoro-1,4-dihydro-2H-pyrano[3,4-c]isoquinolin-1-yl)-3-(3-(difluoromethyl)-4-fluorophenyl)-1-methylurea;
1-(5-(2-aminoethyl)-8,9-difluoro-6-oxo-1,4,5,6-tetrahydro-2H-pyrano[3,4-c]isoquinolin-1-yl)-3-(3-(difluoromethyl)-4-fluorophenyl)-1-methylurea;
3-(3-chloro-4-fluorophenyl)-1-(8,9-difluoro-5-(2-hydroxyethyl)-6-oxo-1,4,5,6-tetrahydro-2H-pyrano[3,4-c]isoquinolin-1-yl)-1-methylurea;
3-(3-chloro-4-fluorophenyl)-1-(8,9-difluoro-6-(2-hydroxyethoxy)-1,4-dihydro-2H-pyrano[3,4-c]isoquinolin-1-yl)-1-methylurea;
1-(8,9-difluoro-6-(2-hydroxyethoxy)-1,4-dihydro-2H-pyrano[3,4-c]isoquinolin-1-yl)-3-(3-(difluoromethyl)-4-fluorophenyl)-1-methylurea;
1-(5-(2-aminoethyl)-8,9-difluoro-6-oxo-1,4,5,6-tetrahydro-2H-pyrano[3,4-c]isoquinolin-1-yl)-3-(3-chloro-4-fluorophenyl)-1-methylurea;
1-(6-(2-aminoethoxy)-8,9-difluoro-1,4-dihydro-2H-pyrano[3,4-c]isoquinolin-1-yl)-3-(3-chloro-4-fluorophenyl)-1-methylurea; or a salt, solvate, prodrug, isotopically labelled derivative, stereoisomer, or tautomer thereof, or any mixtures thereof.
Embodiment 19 provides the compound of any one of Embodiments 1-18, which is at least one selected from the group consisting of:
(R)-3-(3-chloro-4-fluorophenyl)-1-isobutyl-1-(2-oxo-4-(trifluoromethyl)-1,2,5,6,7,8-hexahydroquinolin-5-yl)urea;
(S)-3-(3-chloro-4-fluorophenyl)-1-isobutyl-1-(2-oxo-4-(trifluoromethyl)-1,2,5,6,7,8-hexahydroquinolin-5-yl)urea;
(R)-3-(3-chloro-4-fluorophenyl)-1-methyl-1-(2-oxo-4-(trifluoromethyl)-1,2,5,6,7,8-hexahydroquinolin-5-yl)urea;
(S)-3-(3-chloro-4-fluorophenyl)-1-methyl-1-(2-oxo-4-(trifluoromethyl)-1,2,5,6,7,8-hexahydroquinolin-5-yl)urea;
(R)-3-(3-chloro-4-fluorophenyl)-1-(3-hydroxypropyl)-1-(2-oxo-4-(trifluoromethyl)-1,2,5,6,7,8-hexahydroquinolin-5-yl)urea;
(S)-3-(3-chloro-4-fluorophenyl)-1-(3-hydroxypropyl)-1-(2-oxo-4-(trifluoromethyl)-1,2,5,6,7,8-hexahydroquinolin-5-yl)urea;
(R)-3-(3-chloro-4-fluorophenyl)-1-isobutyl-1-(2-oxo-4-(trifluoromethyl)-2,5,6,7-tetrahydro-1H-cyclopenta[b]pyridin-5-yl)urea;
(S)-3-(3-chloro-4-fluorophenyl)-1-isobutyl-1-(2-oxo-4-(trifluoromethyl)-2,5,6,7-tetrahydro-1H-cyclopenta[b]pyridin-5-yl)urea;
(R)-3-(3,4-difluorophenyl)-1-isobutyl-1-(2-oxo-4-(trifluoromethyl)-2,5,6,7-tetrahydro-1H-cyclopenta[b]pyridin-5-yl)urea;
(S)-3-(3,4-difluorophenyl)-1-isobutyl-1-(2-oxo-4-(trifluoromethyl)-2,5,6,7-tetrahydro-1H-cyclopenta[b]pyridin-5-yl)urea;
(R)-3-(3-chloro-4-fluorophenyl)-1-methyl-1-(6-oxo-1,2,3,4,5,6-hexahydrophenanthridin-1-yl)urea;
(S)-3-(3-chloro-4-fluorophenyl)-1-methyl-1-(6-oxo-1,2,3,4,5,6-hexahydrophenanthridin-1-yl)urea;
(R)-3-(3-chloro-4-fluorophenyl)-1-(3-hydroxypropyl)-1-(6-oxo-1,2,3,4,5,6-hexahydrophenanthridin-1-yl)urea;
(S)-3-(3-chloro-4-fluorophenyl)-1-(3-hydroxypropyl)-1-(6-oxo-1,2,3,4,5,6-hexahydrophenanthridin-1-yl)urea;
(R)-3-(3-chloro-4-fluorophenyl)-1-isobutyl-1-(6-oxo-1,2,3,4,5,6-hexahydrophenanthridin-1-yl)urea;
(S)-3-(3-chloro-4-fluorophenyl)-1-isobutyl-1-(6-oxo-1,2,3,4,5,6-hexahydrophenanthridin-1-yl)urea;
(R)-3-(3-chloro-4-fluorophenyl)-1-(8-fluoro-6-oxo-1,2,3,4,5,6-hexahydrophenanthridin-1-yl)-1-methylurea;
(S)-3-(3-chloro-4-fluorophenyl)-1-(8-fluoro-6-oxo-1,2,3,4,5,6-hexahydrophenanthridin-1-yl)-1-methylurea;
(R)-3-(3-chloro-4-fluorophenyl)-1-(8-fluoro-6-oxo-1,2,3,4,5,6-hexahydrophenanthridin-1-yl)-1-isobutylurea;
(S)-3-(3-chloro-4-fluorophenyl)-1-(8-fluoro-6-oxo-1,2,3,4,5,6-hexahydrophenanthridin-1-yl)-1-isobutylurea;
(R)-3-(3-chloro-4-fluorophenyl)-1-(8-fluoro-6-oxo-1,2,3,4,5,6-hexahydrophenanthridin-1-yl)-1-(3-hydroxypropyl)urea;
(S)-3-(3-chloro-4-fluorophenyl)-1-(8-fluoro-6-oxo-1,2,3,4,5,6-hexahydrophenanthridin-1-yl)-1-(3-hydroxypropyl)urea;
(R)-3-(3-chloro-4-fluorophenyl)-1-methyl-1-(5-oxo-2,3,4,5-tetrahydro-1H-cyclopenta[c]isoquinolin-1-yl)urea;
(S)-3-(3-chloro-4-fluorophenyl)-1-methyl-1-(5-oxo-2,3,4,5-tetrahydro-1H-cyclopenta[c]isoquinolin-1-yl)urea;
(R)-3-(3-chloro-4-fluorophenyl)-1-isobutyl-1-(5-oxo-2,3,4,5-tetrahydro-1H-cyclopenta[c]isoquinolin-1-yl)urea;
(S)-3-(3-chloro-4-fluorophenyl)-1-isobutyl-1-(5-oxo-2,3,4,5-tetrahydro-1H-cyclopenta[c]isoquinolin-1-yl)urea;
(R)-3-(3-chloro-4-fluorophenyl)-1-(3-hydroxypropyl)-1-(5-oxo-2,3,4,5-tetrahydro-1H-cyclopenta[c]isoquinolin-1-yl)urea;
(S)-3-(3-chloro-4-fluorophenyl)-1-(3-hydroxypropyl)-1-(5-oxo-2,3,4,5-tetrahydro-1H-cyclopenta[c]isoquinolin-1-yl)urea;
(R)-3-(3-chloro-4-fluorophenyl)-1-(8,9-difluoro-6-oxo-1,2,3,4,5,6-hexahydrophenanthridin-1-yl)-1-methylurea;
(S)-3-(3-chloro-4-fluorophenyl)-1-(8,9-difluoro-6-oxo-1,2,3,4,5,6-hexahydrophenanthridin-1-yl)-1-methylurea;
(R)-3-(3-chloro-4-fluorophenyl)-1-methyl-1-(6-oxo-1,2,3,4,5,6-hexahydrophenanthridin-1-yl)urea;
(S)-3-(3-chloro-4-fluorophenyl)-1-methyl-1-(6-oxo-1,2,3,4,5,6-hexahydrophenanthridin-1-yl)urea;
(R)-3-(3-chloro-4-fluorophenyl)-1-methyl-1-(6-oxo-1,4,5,6-tetrahydro-2H-pyrano[3,4-c]isoquinolin-1-yl)urea;
(S)-3-(3-chloro-4-fluorophenyl)-1-methyl-1-(6-oxo-1,4,5,6-tetrahydro-2H-pyrano[3,4-c]isoquinolin-1-yl)urea;
(R)-1-(8,9-difluoro-6-oxo-1,2,3,4,5,6-hexahydrophenanthridin-1-yl)-3-(4-fluorophenyl)-1-methylurea;
(S)-1-(8,9-difluoro-6-oxo-1,2,3,4,5,6-hexahydrophenanthridin-1-yl)-3-(4-fluorophenyl)-1-methylurea;
(R)-3-(3-chloro-4-fluorophenyl)-1-(8,10-difluoro-6-oxo-1,2,3,4,5,6-hexahydrophenanthridin-1-yl)-1-methylurea;
(S)-3-(3-chloro-4-fluorophenyl)-1-(8,10-difluoro-6-oxo-1,2,3,4,5,6-hexahydrophenanthridin-1-yl)-1-methylurea;
(R)-3-(3-chloro-4-fluorophenyl)-1-methyl-1-(5-oxo-6,7,8,9,10,11-hexahydro-5H-cyclohepta[c]isoquinolin-11-yl)urea;
(S)-3-(3-chloro-4-fluorophenyl)-1-methyl-1-(5-oxo-6,7,8,9,10,11-hexahydro-5H-cyclohepta[c]isoquinolin-11-yl)urea;
(R)-3-(3-chloro-4-fluorophenyl)-1-(3-hydroxypropyl)-1-(5-oxo-6,7,8,9,10,11-hexahydro-5H-cyclohepta[c]isoquinolin-11-yl)urea;
(S)-3-(3-chloro-4-fluorophenyl)-1-(3-hydroxypropyl)-1-(5-oxo-6,7,8,9,10,11-hexahydro-5H-cyclohepta[c]isoquinolin-11-yl)urea;
(R)-3-(3-chloro-4-fluorophenyl)-1-(8-fluoro-6-oxo-1,4,5,6-tetrahydro-2H-pyrano[3,4-c]isoquinolin-1-yl)-1-methylurea;
(S)-3-(3-chloro-4-fluorophenyl)-1-(8-fluoro-6-oxo-1,4,5,6-tetrahydro-2H-pyrano[3,4-c]isoquinolin-1-yl)-1-methylurea;
(R)-3-(3-chloro-4-fluorophenyl)-1-(8,9-difluoro-6-oxo-1,4,5,6-tetrahydro-2H-pyrano[3,4-c]isoquinolin-1-yl)-1-methylurea;
(S)-3-(3-chloro-4-fluorophenyl)-1-(8,9-difluoro-6-oxo-1,4,5,6-tetrahydro-2H-pyrano[3,4-c]isoquinolin-1-yl)-1-methylurea;
(R)-3-(3-chloro-4-fluorophenyl)-1-(8,10-difluoro-6-oxo-1,4,5,6-tetrahydro-2H-pyrano[3,4-c]isoquinolin-1-yl)-1-methylurea;
(S)-3-(3-chloro-4-fluorophenyl)-1-(8,10-difluoro-6-oxo-1,4,5,6-tetrahydro-2H-pyrano[3,4-c]isoquinolin-1-yl)-1-methylurea;
3-(3-chloro-4-fluorophenyl)-1-methyl-(1R)-(3R-methyl-6-oxo-1,2,3,4,5,6-hexahydrophenanthridin-1-yl)urea;
3-(3-chloro-4-fluorophenyl)-1-methyl-(1R)-(3 S-methyl-6-oxo-1,2,3,4,5,6-hexahydrophenanthridin-1-yl)urea;
3-(3-chloro-4-fluorophenyl)-1-methyl-(1S)-(3R-methyl-6-oxo-1,2,3,4,5,6-hexahydrophenanthridin-1-yl)urea;
3-(3-chloro-4-fluorophenyl)-1-methyl-(1S)-(3S-methyl-6-oxo-1,2,3,4,5,6-hexahydrophenanthridin-1-yl)urea;
(R)-3-(3-chloro-4-fluorophenyl)-1-(3,3-dimethyl-6-oxo-1,2,3,4,5,6-hexahydrophenanthridin-1-yl)-1-methylurea;
(S)-3-(3-chloro-4-fluorophenyl)-1-(3,3-dimethyl-6-oxo-1,2,3,4,5,6-hexahydrophenanthridin-1-yl)-1-methylurea;
(R)-3-(3-chloro-4-fluorophenyl)-1-(7,8-difluoro-6-oxo-1,2,3,4,5,6-hexahydrophenanthridin-1-yl)-1-methylurea;
(S)-3-(3-chloro-4-fluorophenyl)-1-(7,8-difluoro-6-oxo-1,2,3,4,5,6-hexahydrophenanthridin-1-yl)-1-methylurea;
(R)-3-(3-chloro-5-fluorophenyl)-1-methyl-1-(6-oxo-1,4,5,6-tetrahydro-2H-pyrano[3,4-c]isoquinolin-1-yl)urea;
(S)-3-(3-chloro-5-fluorophenyl)-1-methyl-1-(6-oxo-1,4,5,6-tetrahydro-2H-pyrano[3,4-c]isoquinolin-1-yl)urea;
(R)-3-(3-chloro-4-fluorophenyl)-1-isobutyl-1-(6-oxo-1,4,5,6-tetrahydro-2H-pyrano[3,4-c]isoquinolin-1-yl)urea;
(S)-3-(3-chloro-4-fluorophenyl)-1-isobutyl-1-(6-oxo-1,4,5,6-tetrahydro-2H-pyrano[3,4-c]isoquinolin-1-yl)urea;
(R)-3-(3-chloro-4-fluorophenyl)-1-methyl-1-(5-methyl-6-oxo-1,2,3,4,5,6-hexahydrophenanthridin-1-yl)urea;
(S)-3-(3-chloro-4-fluorophenyl)-1-methyl-1-(5-methyl-6-oxo-1,2,3,4,5,6-hexahydrophenanthridin-1-yl)urea;
(R)-3-(3,4-difluorophenyl)-1-(8-fluoro-6-oxo-1,2,3,4,5,6-hexahydrophenanthridin-1-yl)-1-methylurea;
(S)-3-(3,4-difluorophenyl)-1-(8-fluoro-6-oxo-1,2,3,4,5,6-hexahydrophenanthridin-1-yl)-1-methylurea;
(R)-1-(8-fluoro-6-oxo-1,2,3,4,5,6-hexahydrophenanthridin-1-yl)-1-methyl-3-(3,4,5-trifluorophenyl)urea;
(S)-1-(8-fluoro-6-oxo-1,2,3,4,5,6-hexahydrophenanthridin-1-yl)-1-methyl-3-(3,4,5-trifluorophenyl)urea;
(R)-3-(3-chloro-4-fluorophenyl)-1-(6-methoxy-1,2,3,4-tetrahydrophenanthridin-1-yl)-1-methylurea;
(S)-3-(3-chloro-4-fluorophenyl)-1-(6-methoxy-1,2,3,4-tetrahydrophenanthridin-1-yl)-1-methylurea;
(R)-3-(3-chloro-4-fluorophenyl)-1-(7,8-difluoro-6-oxo-1,4,5,6-tetrahydro-2H-pyrano[3,4-c]isoquinolin-1-yl)-1-methylurea;
(S)-3-(3-chloro-4-fluorophenyl)-1-(7,8-difluoro-6-oxo-1,4,5,6-tetrahydro-2H-pyrano[3,4-c]isoquinolin-1-yl)-1-methylurea;
(R)-3-(3-chloro-4-fluorophenyl)-1-(8-fluoro-6-oxo-1,4,5,6-tetrahydro-2H-pyrano[3,4-c]isoquinolin-1-yl)-1-isobutylurea;
(S)-3-(3-chloro-4-fluorophenyl)-1-(8-fluoro-6-oxo-1,4,5,6-tetrahydro-2H-pyrano[3,4-c]isoquinolin-1-yl)-1-isobutylurea;
(R)-3-(3-chloro-4-fluorophenyl)-1-ethyl-1-(8-fluoro-6-oxo-1,4,5,6-tetrahydro-2H-pyrano[3,4-c]isoquinolin-1-yl)urea;
(S)-3-(3-chloro-4-fluorophenyl)-1-ethyl-1-(8-fluoro-6-oxo-1,4,5,6-tetrahydro-2H-pyrano[3,4-c]isoquinolin-1-yl)urea;
(R)-3-(3-chloro-4-fluorophenyl)-1-(8,9-difluoro-6-oxo-1,4,5,6-tetrahydro-2H-pyrano[3,4-c]isoquinolin-1-yl)-1-ethylurea;
(S)-3-(3-chloro-4-fluorophenyl)-1-(8,9-difluoro-6-oxo-1,4,5,6-tetrahydro-2H-pyrano[3,4-c]isoquinolin-1-yl)-1-ethylurea;
(R)-3-(3-chloro-4-fluorophenyl)-1-methyl-1-(6-oxo-1,2,3,4,5,6,7,8,9,10-decahydrophenanthridin-1-yl)urea;
(S)-3-(3-chloro-4-fluorophenyl)-1-methyl-1-(6-oxo-1,2,3,4,5,6,7,8,9,10-decahydrophenanthridin-1-yl)urea;
(R)-3-(3,4-difluorophenyl)-1-methyl-1-(6-oxo-1,2,3,4,5,6,7,8,9,10-decahydrophenanthridin-1-yl)urea;
(S)-3-(3,4-difluorophenyl)-1-methyl-1-(6-oxo-1,2,3,4,5,6,7,8,9,10-decahydrophenanthridin-1-yl)urea;
(R)-3-(3,4-difluorophenyl)-1-ethyl-1-(6-oxo-1,2,3,4,5,6,7,8,9,10-decahydrophenanthridin-1-yl)urea;
(S)-3-(3,4-difluorophenyl)-1-ethyl-1-(6-oxo-1,2,3,4,5,6,7,8,9,10-decahydrophenanthridin-1-yl)urea;
(R)-3-(3-chloro-4-fluorophenyl)-1-ethyl-1-(6-oxo-1,2,3,4,5,6,7,8,9,10-decahydrophenanthridin-1-yl)urea;
(S)-3-(3-chloro-4-fluorophenyl)-1-ethyl-1-(6-oxo-1,2,3,4,5,6,7,8,9,10-decahydrophenanthridin-1-yl)urea;
(R)-3-(3-chloro-4-fluorophenyl)-1-(8-chloro-6-oxo-1,4,5,6-tetrahydro-2H-pyrano[3,4-c]isoquinolin-1-yl)-1-methylurea;
(S)-3-(3-chloro-4-fluorophenyl)-1-(8-chloro-6-oxo-1,4,5,6-tetrahydro-2H-pyrano[3,4-c]isoquinolin-1-yl)-1-methylurea;
(R)-3-(3-chloro-4-fluorophenyl)-1-(8-chloro-6-oxo-1,4,5,6-tetrahydro-2H-pyrano[3,4-c]isoquinolin-1-yl)-1-ethylurea;
(S)-3-(3-chloro-4-fluorophenyl)-1-(8-chloro-6-oxo-1,4,5,6-tetrahydro-2H-pyrano[3,4-c]isoquinolin-1-yl)-1-ethylurea;
(R)-3-(4-fluoro-3-methylphenyl)-1-(8-fluoro-6-oxo-1,4,5,6-tetrahydro-2H-pyrano[3,4-c]isoquinolin-1-yl)-1-methylurea;
(S)-3-(4-fluoro-3-methylphenyl)-1-(8-fluoro-6-oxo-1,4,5,6-tetrahydro-2H-pyrano[3,4-c]isoquinolin-1-yl)-1-methylurea;
(R)-1-(8-chloro-6-oxo-1,4,5,6-tetrahydro-2H-pyrano[3,4-c]isoquinolin-1-yl)-3-(4-fluoro-3-methylphenyl)-1-methylurea;
(S)-1-(8-chloro-6-oxo-1,4,5,6-tetrahydro-2H-pyrano[3,4-c]isoquinolin-1-yl)-3-(4-fluoro-3-methylphenyl)-1-methylurea;
(R)-1-(8-chloro-6-oxo-1,4,5,6-tetrahydro-2H-pyrano[3,4-c]isoquinolin-1-yl)-1-ethyl-3-(4-fluoro-3-methylphenyl)urea;
(S)-1-(8-chloro-6-oxo-1,4,5,6-tetrahydro-2H-pyrano[3,4-c]isoquinolin-1-yl)-1-ethyl-3-(4-fluoro-3-methylphenyl)urea;
(R)-3-(3-chloro-4-fluorophenyl)-1-methyl-1-(5-oxo-1,4,5,6,7,8,9,10-octahydro-2H-pyrano[3,4-c]quinolin-10-yl)urea;
(S)-3-(3-chloro-4-fluorophenyl)-1-methyl-1-(5-oxo-1,4,5,6,7,8,9,10-octahydro-2H-pyrano[3,4-c]quinolin-10-yl)urea;
(R)-3-(3,4-difluorophenyl)-1-methyl-1-(5-oxo-1,4,5,6,7,8,9,10-octahydro-2H-pyrano[3,4-c]quinolin-10-yl)urea;
(S)-3-(3,4-difluorophenyl)-1-methyl-1-(5-oxo-1,4,5,6,7,8,9,10-octahydro-2H-pyrano[3,4-c]quinolin-10-yl)urea;
(R)-3-(3-cyano-4-fluorophenyl)-1-(8-fluoro-6-oxo-1,4,5,6-tetrahydro-2H-pyrano[3,4-c]isoquinolin-1-yl)-1-methylurea;
(S)-3-(3-cyano-4-fluorophenyl)-1-(8-fluoro-6-oxo-1,4,5,6-tetrahydro-2H-pyrano[3,4-c]isoquinolin-1-yl)-1-methylurea;
(R)-1-(8-chloro-6-oxo-1,4,5,6-tetrahydro-2H-pyrano[3,4-c]isoquinolin-1-yl)-3-(3-cyano-4-fluorophenyl)-1-methylurea;
(S)-1-(8-chloro-6-oxo-1,4,5,6-tetrahydro-2H-pyrano[3,4-c]isoquinolin-1-yl)-3-(3-cyano-4-fluorophenyl)-1-methylurea;
(R)-1-(8-chloro-6-oxo-1,4,5,6-tetrahydro-2H-pyrano[3,4-c]isoquinolin-1-yl)-3-(3-cyano-4-fluorophenyl)-1-ethylurea;
(S)-1-(8-chloro-6-oxo-1,4,5,6-tetrahydro-2H-pyrano[3,4-c]isoquinolin-1-yl)-3-(3-cyano-4-fluorophenyl)-1-ethylurea;
(R)-1-(3-chloro-4-fluorophenyl)-3-(8-fluoro-6-oxo-1,4,5,6-tetrahydro-2H-pyrano[3,4-c]isoquinolin-1-yl)urea;
(S)-1-(3-chloro-4-fluorophenyl)-3-(8-fluoro-6-oxo-1,4,5,6-tetrahydro-2H-pyrano[3,4-c]isoquinolin-1-yl)urea;
(R)-1-(3-chloro-4-fluorophenyl)-3-(8,9-difluoro-6-oxo-1,4,5,6-tetrahydro-2H-pyrano[3,4-c]isoquinolin-1-yl)urea;
(S)-1-(3-chloro-4-fluorophenyl)-3-(8,9-difluoro-6-oxo-1,4,5,6-tetrahydro-2H-pyrano[3,4-c]isoquinolin-1-yl)urea;
(R)-1-(8,9-difluoro-6-oxo-1,4,5,6-tetrahydro-2H-pyrano[3,4-c]isoquinolin-1-yl)-3-(4-fluoro-3-methylphenyl)-1-methylurea;
(S)-1-(8,9-difluoro-6-oxo-1,4,5,6-tetrahydro-2H-pyrano[3,4-c]isoquinolin-1-yl)-3-(4-fluoro-3-methylphenyl)-1-methylurea;
(R)-3-(3-chloro-4-fluorophenyl)-1-(9-fluoro-6-oxo-1,4,5,6-tetrahydro-2H-pyrano[3,4-c]isoquinolin-1-yl)-1-methylurea;
(S)-3-(3-chloro-4-fluorophenyl)-1-(9-fluoro-6-oxo-1,4,5,6-tetrahydro-2H-pyrano[3,4-c]isoquinolin-1-yl)-1-methylurea;
(R)-3-(3-chloro-4-fluorophenyl)-1-ethyl-1-(9-fluoro-6-oxo-1,4,5,6-tetrahydro-2H-pyrano[3,4-c]isoquinolin-1-yl)urea;
(S)-3-(3-chloro-4-fluorophenyl)-1-ethyl-1-(9-fluoro-6-oxo-1,4,5,6-tetrahydro-2H-pyrano[3,4-c]isoquinolin-1-yl)urea;
(R)-3-(4-fluoro-3-methylphenyl)-1-(9-fluoro-6-oxo-1,4,5,6-tetrahydro-2H-pyrano[3,4-c]isoquinolin-1-yl)-1-methylurea;
(S)-3-(4-fluoro-3-methylphenyl)-1-(9-fluoro-6-oxo-1,4,5,6-tetrahydro-2H-pyrano[3,4-c]isoquinolin-1-yl)-1-methylurea;
(R)-1-ethyl-3-(4-fluoro-3-methylphenyl)-1-(9-fluoro-6-oxo-1,4,5,6-tetrahydro-2H-pyrano[3,4-c]isoquinolin-1-yl)urea;
(S)-1-ethyl-3-(4-fluoro-3-methylphenyl)-1-(9-fluoro-6-oxo-1,4,5,6-tetrahydro-2H-pyrano[3,4-c]isoquinolin-1-yl)urea;
(R)-3-(3-cyano-4-fluorophenyl)-1-(9-fluoro-6-oxo-1,4,5,6-tetrahydro-2H-pyrano[3,4-c]isoquinolin-1-yl)-1-methylurea;
(S)-3-(3-cyano-4-fluorophenyl)-1-(9-fluoro-6-oxo-1,4,5,6-tetrahydro-2H-pyrano[3,4-c]isoquinolin-1-yl)-1-methylurea;
(R)-3-(3-cyano-4-fluorophenyl)-1-ethyl-1-(9-fluoro-6-oxo-1,4,5,6-tetrahydro-2H-pyrano[3,4-c]isoquinolin-1-yl)urea;
(S)-3-(3-cyano-4-fluorophenyl)-1-ethyl-1-(9-fluoro-6-oxo-1,4,5,6-tetrahydro-2H-pyrano[3,4-c]isoquinolin-1-yl)urea;
(R)-3-(3-chloro-4-fluorophenyl)-1-methyl-1-(5-oxo-3,4,5,6,7,8,9,10-octahydro-1H-pyrano[4,3-c]quinolin-10-yl)urea;
(S)-3-(3-chloro-4-fluorophenyl)-1-methyl-1-(5-oxo-3,4,5,6,7,8,9,10-octahydro-1H-pyrano[4,3-c]quinolin-10-yl)urea;
(R)-3-(3,4-difluorophenyl)-1-methyl-1-(5-oxo-3,4,5,6,7,8,9,10-octahydro-1H-pyrano[4,3-c]quinolin-10-yl)urea;
(S)-3-(3,4-difluorophenyl)-1-methyl-1-(5-oxo-3,4,5,6,7,8,9,10-octahydro-1H-pyrano[4,3-c]quinolin-10-yl)urea;
(R)-3-(3-chloro-4-fluorophenyl)-1-methyl-1-(5-oxo-4,5,6,7,9,10-hexahydro-1H,3H-dipyrano[3,4-b:3′,4′-d]pyridin-10-yl)urea;
(S)-3-(3-chloro-4-fluorophenyl)-1-methyl-1-(5-oxo-4,5,6,7,9,10-hexahydro-1H,3H-dipyrano[3,4-b:3′,4′-d]pyridin-10-yl)urea;
(R)-3-(3,4-difluorophenyl)-1-methyl-1-(5-oxo-4,5,6,7,9,10-hexahydro-1H,3H-dipyrano[3,4-b:3′,4′-d]pyridin-10-yl)urea;
(S)-3-(3,4-difluorophenyl)-1-methyl-1-(5-oxo-4,5,6,7,9,10-hexahydro-1H,3H-dipyrano[3,4-b:3′,4′-d]pyridin-10-yl)urea;
(R)-3-(3-chloro-4-fluorophenyl)-1-(8-cyano-6-oxo-1,4,5,6-tetrahydro-2H-pyrano[3,4-c]isoquinolin-1-yl)-1-methylurea;
(S)-3-(3-chloro-4-fluorophenyl)-1-(8-cyano-6-oxo-1,4,5,6-tetrahydro-2H-pyrano[3,4-c]isoquinolin-1-yl)-1-methylurea;
(R)-3-(3-chloro-4-fluorophenyl)-1-(8-cyano-6-oxo-1,4,5,6-tetrahydro-2H-pyrano[3,4-c]isoquinolin-1-yl)-1-ethylurea;
(S)-3-(3-chloro-4-fluorophenyl)-1-(8-cyano-6-oxo-1,4,5,6-tetrahydro-2H-pyrano[3,4-c]isoquinolin-1-yl)-1-ethylurea;
(R)-3-(3-chloro-4-fluorophenyl)-1-methyl-1-(6-oxo-1,2,4,5,6,7,9,10-octahydrodipyrano[3,4-b:4′,3′-d]pyridin-1-yl)urea;
(S)-3-(3-chloro-4-fluorophenyl)-1-methyl-1-(6-oxo-1,2,4,5,6,7,9,10-octahydrodipyrano[3,4-b:4′,3′-d]pyridin-1-yl)urea;
(R)-3-(3,4-difluorophenyl)-1-methyl-1-(6-oxo-1,2,4,5,6,7,9,10-octahydrodipyrano[3,4-b:4′,3′-d]pyridin-1-yl)urea;
(S)-3-(3,4-difluorophenyl)-1-methyl-1-(6-oxo-1,2,4,5,6,7,9,10-octahydrodipyrano[3,4-b:4′,3′-d]pyridin-1-yl)urea;
(R)-3-(3-chloro-4-fluorophenyl)-1-methyl-1-(6-oxo-1,4,5,6-tetrahydro-2H-pyrano[3,4-b]thieno[3,2-d]pyridin-1-yl)urea;
(S)-3-(3-chloro-4-fluorophenyl)-1-methyl-1-(6-oxo-1,4,5,6-tetrahydro-2H-pyrano[3,4-b]thieno[3,2-d]pyridin-1-yl)urea;
(R)-3-(3-chloro-4-fluorophenyl)-1-methyl-1-(4-oxo-4,6,8,9-tetrahydro-5H-pyrano[3,4-b]thieno[2,3-d]pyridin-9-yl)urea;
(S)-3-(3-chloro-4-fluorophenyl)-1-methyl-1-(4-oxo-4,6,8,9-tetrahydro-5H-pyrano[3,4-b]thieno[2,3-d]pyridin-9-yl)urea;
(R)-3-(3,5-dichloro-4-fluorophenyl)-1-(8-fluoro-6-oxo-1,4,5,6-tetrahydro-2H-pyrano[3,4-c]isoquinolin-1-yl)-1-methylurea;
(S)-3-(3,5-dichloro-4-fluorophenyl)-1-(8-fluoro-6-oxo-1,4,5,6-tetrahydro-2H-pyrano[3,4-c]isoquinolin-1-yl)-1-methylurea;
(R)-3-(3,4-difluorophenyl)-1-(8-fluoro-6-oxo-1,4,5,6-tetrahydro-2H-pyrano[3,4-c]isoquinolin-1-yl)-1-isobutylurea;
(S)-3-(3,4-difluorophenyl)-1-(8-fluoro-6-oxo-1,4,5,6-tetrahydro-2H-pyrano[3,4-c]isoquinolin-1-yl)-1-isobutylurea;
(R)-1-(8,9-difluoro-6-oxo-1,4,5,6-tetrahydro-2H-pyrano[3,4-c]isoquinolin-1-yl)-1-methyl-3-phenylurea;
(S)-1-(8,9-difluoro-6-oxo-1,4,5,6-tetrahydro-2H-pyrano[3,4-c]isoquinolin-1-yl)-1-methyl-3-phenylurea;
(R)-1-(8,9-difluoro-6-oxo-1,4,5,6-tetrahydro-2H-pyrano[3,4-c]isoquinolin-1-yl)-3-(4-fluorophenyl)-1-methylurea;
(S)-1-(8,9-difluoro-6-oxo-1,4,5,6-tetrahydro-2H-pyrano[3,4-c]isoquinolin-1-yl)-3-(4-fluorophenyl)-1-methylurea;
(R)-3-(3-chloro-4-fluorophenyl)-1-methyl-1-(4-oxo-4,5,8,9-tetrahydro-6H-pyrano[3,4-b]thieno[3,4-d]pyridin-9-yl)urea;
(S)-3-(3-chloro-4-fluorophenyl)-1-methyl-1-(4-oxo-4,5,8,9-tetrahydro-6H-pyrano[3,4-b]thieno[3,4-d]pyridin-9-yl)urea;
(R)-3-(3-cyano-4-fluorophenyl)-1-(8,9-difluoro-6-oxo-1,4,5,6-tetrahydro-2H-pyrano[3,4-c]isoquinolin-1-yl)-1-methylurea;
(S)-3-(3-cyano-4-fluorophenyl)-1-(8,9-difluoro-6-oxo-1,4,5,6-tetrahydro-2H-pyrano[3,4-c]isoquinolin-1-yl)-1-methylurea;
(R)-3-(3-chloro-4,5-difluorophenyl)-1-(8-fluoro-6-oxo-1,4,5,6-tetrahydro-2H-pyrano[3,4-c]isoquinolin-1-yl)-1-methylurea;
(S)-3-(3-chloro-4,5-difluorophenyl)-1-(8-fluoro-6-oxo-1,4,5,6-tetrahydro-2H-pyrano[3,4-c]isoquinolin-1-yl)-1-methylurea;
(R)-3-(3-chloro-4-fluorophenyl)-1-(8-fluoro-6-oxo-1,4,5,6-tetrahydro-2H-pyrano[3,4-c]isoquinolin-1-yl)-1-(3-hydroxypropyl)urea;
(S)-3-(3-chloro-4-fluorophenyl)-1-(8-fluoro-6-oxo-1,4,5,6-tetrahydro-2H-pyrano[3,4-c]isoquinolin-1-yl)-1-(3-hydroxypropyl)urea;
(R)-3-(3-chloro-4-fluorophenyl)-1-(8-fluoro-6-oxo-1,4,5,6-tetrahydro-2H-pyrano[3,4-c]isoquinolin-1-yl)-1-(2-hydroxy-2-methylpropyl)urea;
(S)-3-(3-chloro-4-fluorophenyl)-1-(8-fluoro-6-oxo-1,4,5,6-tetrahydro-2H-pyrano[3,4-c]isoquinolin-1-yl)-1-(2-hydroxy-2-methylpropyl)urea;
(R)-1-(8-fluoro-6-oxo-1,4,5,6-tetrahydro-2H-pyrano[3,4-c]isoquinolin-1-yl)-1-isobutyl-3-(3,4,5-trifluorophenyl)urea;
(S)-1-(8-fluoro-6-oxo-1,4,5,6-tetrahydro-2H-pyrano[3,4-c]isoquinolin-1-yl)-1-isobutyl-3-(3,4,5-trifluorophenyl)urea;
(R)-3-(3-cyano-4-fluorophenyl)-1-(8-fluoro-6-oxo-1,4,5,6-tetrahydro-2H-pyrano[3,4-c]isoquinolin-1-yl)-1-isobutylurea;
(S)-3-(3-cyano-4-fluorophenyl)-1-(8-fluoro-6-oxo-1,4,5,6-tetrahydro-2H-pyrano[3,4-c]isoquinolin-1-yl)-1-isobutylurea;
(R)-1-(8,9-difluoro-6-oxo-1,4,5,6-tetrahydro-2H-pyrano[3,4-c]isoquinolin-1-yl)-3-(3,4-difluorophenyl)-1-methylurea;
(S)-1-(8,9-difluoro-6-oxo-1,4,5,6-tetrahydro-2H-pyrano[3,4-c]isoquinolin-1-yl)-3-(3,4-difluorophenyl)-1-methylurea;
(R)-3-(3-chlorophenyl)-1-(8,9-difluoro-6-oxo-1,4,5,6-tetrahydro-2H-pyrano[3,4-c]isoquinolin-1-yl)-1-methylurea;
(S)-3-(3-chlorophenyl)-1-(8,9-difluoro-6-oxo-1,4,5,6-tetrahydro-2H-pyrano[3,4-c]isoquinolin-1-yl)-1-methylurea;
(R)-3-(3-cyano-4-fluorophenyl)-1-(8,9-difluoro-6-oxo-1,4,5,6-tetrahydro-2H-pyrano[3,4-c]isoquinolin-1-yl)-1-isobutylurea;
(S)-3-(3-cyano-4-fluorophenyl)-1-(8,9-difluoro-6-oxo-1,4,5,6-tetrahydro-2H-pyrano[3,4-c]isoquinolin-1-yl)-1-isobutylurea;
(R)-3-(3-(difluoromethyl)-4-fluorophenyl)-1-(8-fluoro-6-oxo-1,4,5,6-tetrahydro-2H-pyrano[3,4-c]isoquinolin-1-yl)-1-methylurea;
(S)-3-(3-(difluoromethyl)-4-fluorophenyl)-1-(8-fluoro-6-oxo-1,4,5,6-tetrahydro-2H-pyrano[3,4-c]isoquinolin-1-yl)-1-methylurea;
(R)-1-(8,9-difluoro-6-oxo-1,4,5,6-tetrahydro-2H-pyrano[3,4-c]isoquinolin-1-yl)-3-(3-(difluoromethyl)-4-fluorophenyl)-1-methylurea;
(S)-1-(8,9-difluoro-6-oxo-1,4,5,6-tetrahydro-2H-pyrano[3,4-c]isoquinolin-1-yl)-3-(3-(difluoromethyl)-4-fluorophenyl)-1-methylurea;
(R)-3-(4-fluoro-3-methylphenyl)-1-(8-fluoro-6-oxo-1,4,5,6-tetrahydro-2H-pyrano[3,4-c]isoquinolin-1-yl)-1-isobutylurea;
(S)-3-(4-fluoro-3-methylphenyl)-1-(8-fluoro-6-oxo-1,4,5,6-tetrahydro-2H-pyrano[3,4-c]isoquinolin-1-yl)-1-isobutylurea;
(R)-1-(8,9-difluoro-6-oxo-1,4,5,6-tetrahydro-2H-pyrano[3,4-c]isoquinolin-1-yl)-1-methyl-3-(3,4,5-trifluorophenyl)urea;
(S)-1-(8,9-difluoro-6-oxo-1,4,5,6-tetrahydro-2H-pyrano[3,4-c]isoquinolin-1-yl)-1-methyl-3-(3,4,5-trifluorophenyl)urea;
(R)-1-(3-cyano-4-fluorophenyl)-3-(8,9-difluoro-6-oxo-1,4,5,6-tetrahydro-2H-pyrano[3,4-c]isoquinolin-1-yl)urea;
(S)-1-(3-cyano-4-fluorophenyl)-3-(8,9-difluoro-6-oxo-1,4,5,6-tetrahydro-2H-pyrano[3,4-c]isoquinolin-1-yl)urea;
(R)-2-(3-(3-chloro-4-fluorophenyl)-1-(8,9-difluoro-6-oxo-1,4,5,6-tetrahydro-2H-pyrano[3,4-c]isoquinolin-1-yl)ureido)ethane-1-sulfonamide;
(S)-2-(3-(3-chloro-4-fluorophenyl)-1-(8,9-difluoro-6-oxo-1,4,5,6-tetrahydro-2H-pyrano[3,4-c]isoquinolin-1-yl)ureido)ethane-1-sulfonamide;
(R)-3-(3-cyano-4-fluorophenyl)-1-(8,9-difluoro-6-oxo-1,4,5,6-tetrahydro-2H-pyrano[3,4-c]isoquinolin-1-yl)-1-ethylurea;
(S)-3-(3-cyano-4-fluorophenyl)-1-(8,9-difluoro-6-oxo-1,4,5,6-tetrahydro-2H-pyrano[3,4-c]isoquinolin-1-yl)-1-ethylurea;
(R)-3-(3-chloro-4-fluorophenyl)-1-(8,9-difluoro-6-oxo-1,4,5,6-tetrahydro-2H-pyrano[3,4-c]isoquinolin-1-yl)-1-(2-(methylsulfonyl)ethyl)urea;
(S)-3-(3-chloro-4-fluorophenyl)-1-(8,9-difluoro-6-oxo-1,4,5,6-tetrahydro-2H-pyrano[3,4-c]isoquinolin-1-yl)-1-(2-(methylsulfonyl)ethyl)urea;
(R)-3-(4-chloro-3-fluorophenyl)-1-(8,9-difluoro-6-oxo-1,4,5,6-tetrahydro-2H-pyrano[3,4-c]isoquinolin-1-yl)-1-methylurea;
(S)-3-(4-chloro-3-fluorophenyl)-1-(8,9-difluoro-6-oxo-1,4,5,6-tetrahydro-2H-pyrano[3,4-c]isoquinolin-1-yl)-1-methylurea;
(R)-3-(4-chloro-3-cyanophenyl)-1-(8,9-difluoro-6-oxo-1,4,5,6-tetrahydro-2H-pyrano[3,4-c]isoquinolin-1-yl)-1-methylurea;
(S)-3-(4-chloro-3-cyanophenyl)-1-(8,9-difluoro-6-oxo-1,4,5,6-tetrahydro-2H-pyrano[3,4-c]isoquinolin-1-yl)-1-methylurea;
(R)-3-(3,4-dichlorophenyl)-1-(8,9-difluoro-6-oxo-1,4,5,6-tetrahydro-2H-pyrano[3,4-c]isoquinolin-1-yl)-1-methylurea;
(S)-3-(3,4-dichlorophenyl)-1-(8,9-difluoro-6-oxo-1,4,5,6-tetrahydro-2H-pyrano[3,4-c]isoquinolin-1-yl)-1-methylurea;
(R)-1-(8,9-difluoro-6-oxo-1,2,3,4,5,6-hexahydrophenanthridin-1-yl)-1-methyl-3-(1-(trifluoromethyl)cyclopropyl)urea;
(S)-1-(8,9-difluoro-6-oxo-1,2,3,4,5,6-hexahydrophenanthridin-1-yl)-1-methyl-3-(1-(trifluoromethyl)cyclopropyl)urea;
(R)-1-(8,9-difluoro-6-oxo-1,4,5,6-tetrahydro-2H-pyrano[3,4-c]isoquinolin-1-yl)-1-methyl-3-(1-(trifluoromethyl)cyclopropyl)urea;
(S)-1-(8,9-difluoro-6-oxo-1,4,5,6-tetrahydro-2H-pyrano[3,4-c]isoquinolin-1-yl)-1-methyl-3-(1-(trifluoromethyl)cyclopropyl)urea;
(R)-3-(3-Chloro-4-fluorophenyl)-1-(8,9-difluoro-6-oxo-1,4,5,6-tetrahydro-2H-pyrano[3,4-c]isoquinolin-1-yl-1-d)-1-(methyl-d3)urea;
(S)-3-(3-Chloro-4-fluorophenyl)-1-(8,9-difluoro-6-oxo-1,4,5,6-tetrahydro-2H-pyrano[3,4-c]isoquinolin-1-yl-1-d)-1-(methyl-d3)urea;
(R)-3-(3-Chloro-4-methoxyphenyl)-1-(8,9-difluoro-6-oxo-1,4,5,6-tetrahydro-2H-pyrano[3,4-c]isoquinolin-1-yl)-1-methylurea;
(S)-3-(3-Chloro-4-methoxyphenyl)-1-(8,9-difluoro-6-oxo-1,4,5,6-tetrahydro-2H-pyrano[3,4-c]isoquinolin-1-yl)-1-methylurea;
(R)-3-(3-Chloro-4-hydroxyphenyl)-1-(8,9-difluoro-6-oxo-1,4,5,6-tetrahydro-2H-pyrano[3,4-c]isoquinolin-1-yl)-1-methylurea;
(S)-3-(3-Chloro-4-hydroxyphenyl)-1-(8,9-difluoro-6-oxo-1,4,5,6-tetrahydro-2H-pyrano[3,4-c]isoquinolin-1-yl)-1-methylurea;
(R)-3-(3-Chloro-4-fluorophenyl)-1-(8,9-difluoro-6-oxo-1,2,3,4,5,6-hexahydrobenzo[c][1,7]naphthyridin-1-yl)-1-methylurea;
(S)-3-(3-Chloro-4-fluorophenyl)-1-(8,9-difluoro-6-oxo-1,2,3,4,5,6-hexahydrobenzo[c][1,7]naphthyridin-1-yl)-1-methylurea;
3-(3-Chloro-4-fluorophenyl)-(1R)-(8,9-difluoro-4R-hydroxy-6-oxo-1,4,5,6-tetrahydro-2H-pyrano[3,4-c]isoquinolin-1-yl)-1-methylurea;
3-(3-Chloro-4-fluorophenyl)-(1R)-(8,9-difluoro-4S-hydroxy-6-oxo-1,4,5,6-tetrahydro-2H-pyrano[3,4-c]isoquinolin-1-yl)-1-methylurea;
3-(3-Chloro-4-fluorophenyl)-(1S)-(8,9-difluoro-4R-hydroxy-6-oxo-1,4,5,6-tetrahydro-2H-pyrano[3,4-c]isoquinolin-1-yl)-1-methylurea;
3-(3-Chloro-4-fluorophenyl)-(1S)-(8,9-difluoro-4S-hydroxy-6-oxo-1,4,5,6-tetrahydro-2H-pyrano[3,4-c]isoquinolin-1-yl)-1-methylurea;
(R)-3-(3-chloro-4-Fluorophenyl)-1-(8,9-difluoro-4,6-dioxo-1,4,5,6-tetrahydro-2H-pyrano[3,4-c]isoquinolin-1-yl)-1-methylurea;
(S)-3-(3-chloro-4-Fluorophenyl)-1-(8,9-difluoro-4,6-dioxo-1,4,5,6-tetrahydro-2H-pyrano[3,4-c]isoquinolin-1-yl)-1-methylurea;
(R)-3-(3-Chloro-4-fluorophenyl)-1-(8-fluoro-6-oxo-1,2,3,4,5,6-hexahydrobenzo[c][1,7]naphthyridin-1-yl)-1-methylurea;
(S)-3-(3-Chloro-4-fluorophenyl)-1-(8-fluoro-6-oxo-1,2,3,4,5,6-hexahydrobenzo[c][1,7]naphthyridin-1-yl)-1-methylurea;
(R)-3-(3-Chloro-4-fluorophenyl)-1-(8-fluoro-3-methyl-6-oxo-1,2,3,4,5,6-hexahydrobenzo[c][1,7]naphthyridin-1-yl)-1-methylurea;
(S)-3-(3-Chloro-4-fluorophenyl)-1-(8-fluoro-3-methyl-6-oxo-1,2,3,4,5,6-hexahydrobenzo[c][1,7]naphthyridin-1-yl)-1-methylurea;
(R)-1-(3-Acetyl-8-fluoro-6-oxo-1,2,3,4,5,6-hexahydrobenzo[c][1,7]naphthyridin-1-yl)-3-(3-chloro-4-fluorophenyl)-1-methylurea;
(S)-1-(3-Acetyl-8-fluoro-6-oxo-1,2,3,4,5,6-hexahydrobenzo[c][1,7]naphthyridin-1-yl)-3-(3-chloro-4-fluorophenyl)-1-methylurea;
(R)-1-(3-Acetyl-8,9-difluoro-6-oxo-1,2,3,4,5,6-hexahydrobenzo[c][1,7]naphthyridin-1-yl)-3-(3-chloro-4-fluorophenyl)-1-methylurea;
(S)-1-(3-Acetyl-8,9-difluoro-6-oxo-1,2,3,4,5,6-hexahydrobenzo[c][1,7]naphthyridin-1-yl)-3-(3-chloro-4-fluorophenyl)-1-methylurea;
(R)-3-(3-Cyano-4-fluorophenyl)-1-(8-fluoro-6-oxo-1,2,3,4,5,6-hexahydrobenzo[c][1,7]naphthyridin-1-yl)-1-methylurea;
(S)-3-(3-Cyano-4-fluorophenyl)-1-(8-fluoro-6-oxo-1,2,3,4,5,6-hexahydrobenzo[c][1,7]naphthyridin-1-yl)-1-methylurea;
(R)-3-(3-Cyano-4-fluorophenyl)-1-(8-fluoro-3-methyl-6-oxo-1,2,3,4,5,6-hexahydrobenzo[c][1,7]naphthyridin-1-yl)-1-methylurea;
(S)-3-(3-Cyano-4-fluorophenyl)-1-(8-fluoro-3-methyl-6-oxo-1,2,3,4,5,6-hexahydrobenzo[c][1,7]naphthyridin-1-yl)-1-methylurea;
(R)-1-(3-Acetyl-8-fluoro-6-oxo-1,2,3,4,5,6-hexahydrobenzo[c][1,7]naphthyridin-1-yl)-3-(3-cyano-4-fluorophenyl)-1-methylurea;
(S)-1-(3-Acetyl-8-fluoro-6-oxo-1,2,3,4,5,6-hexahydrobenzo[c][1,7]naphthyridin-1-yl)-3-(3-cyano-4-fluorophenyl)-1-methylurea;
(R)-3-(3-Chloro-4-fluorophenyl)-1-(8,9-difluoro-3-methyl-6-oxo-1,2,3,4,5,6-hexahydrobenzo[c][1,7]naphthyridin-1-yl)-1-methylurea;
(S)-3-(3-Chloro-4-fluorophenyl)-1-(8,9-difluoro-3-methyl-6-oxo-1,2,3,4,5,6-hexahydrobenzo[c][1,7]naphthyridin-1-yl)-1-methylurea;
(R)-3-(3-Chloro-4-fluorophenyl)-1-(8-fluoro-3-(2-hydroxyethyl)-6-oxo-1,2,3,4,5,6-hexahydrobenzo[c][1,7]naphthyridin-1-yl)-1-methylurea;
(S)-3-(3-Chloro-4-fluorophenyl)-1-(8-fluoro-3-(2-hydroxyethyl)-6-oxo-1,2,3,4,5,6-hexahydrobenzo[c][1,7]naphthyridin-1-yl)-1-methylurea;
(R)-3-(3-Cyano-4-fluorophenyl)-1-(8-fluoro-3-(2-hydroxyethyl)-6-oxo-1,2,3,4,5,6-hexahydrobenzo[c][1,7]naphthyridin-1-yl)-1-methylurea;
(S)-3-(3-Cyano-4-fluorophenyl)-1-(8-fluoro-3-(2-hydroxyethyl)-6-oxo-1,2,3,4,5,6-hexahydrobenzo[c][1,7]naphthyridin-1-yl)-1-methylurea;
(R)-3-(3-Cyano-4-fluorophenyl)-1-(8,9-difluoro-3-methyl-6-oxo-1,2,3,4,5,6-hexahydrobenzo[c][1,7]naphthyridin-1-yl)-1-methylurea;
(S)-3-(3-Cyano-4-fluorophenyl)-1-(8,9-difluoro-3-methyl-6-oxo-1,2,3,4,5,6-hexahydrobenzo[c][1,7]naphthyridin-1-yl)-1-methylurea;
(R)-3-(3-Cyano-4-fluorophenyl)-1-(8,9-difluoro-6-oxo-1,2,3,4,5,6-hexahydrobenzo[c][1,7]naphthyridin-1-yl)-1-methylurea;
(S)-3-(3-Cyano-4-fluorophenyl)-1-(8,9-difluoro-6-oxo-1,2,3,4,5,6-hexahydrobenzo[c][1,7]naphthyridin-1-yl)-1-methylurea;
(R)-1-(3-Acetyl-8,9-difluoro-6-oxo-1,2,3,4,5,6-hexahydrobenzo[c][1,7]naphthyridin-1-yl)-3-(3-cyano-4-fluorophenyl)-1-methylurea;
(S)-1-(3-Acetyl-8,9-difluoro-6-oxo-1,2,3,4,5,6-hexahydrobenzo[c][1,7]naphthyridin-1-yl)-3-(3-cyano-4-fluorophenyl)-1-methylurea;
(R)-3-(3-Chloro-4-fluorophenyl)-1-(8,9-difluoro-3-(2-hydroxyethyl)-6-oxo-1,2,3,4,5,6-hexahydrobenzo[c][1,7]naphthyridin-1-yl)-1-methylurea;
(S)-3-(3-Chloro-4-fluorophenyl)-1-(8,9-difluoro-3-(2-hydroxyethyl)-6-oxo-1,2,3,4,5,6-hexahydrobenzo[c][1,7]naphthyridin-1-yl)-1-methylurea;
(R)-3-(3-Cyano-4-fluorophenyl)-1-(8,9-difluoro-3-(2-hydroxyethyl)-6-oxo-1,2,3,4,5,6-hexahydrobenzo[c][1,7]naphthyridine-1-yl)-1-methylurea;
(S)-3-(3-Cyano-4-fluorophenyl)-1-(8,9-difluoro-3-(2-hydroxyethyl)-6-oxo-1,2,3,4,5,6-hexahydrobenzo[c][1,7]naphthyridine-1-yl)-1-methylurea;
(R)-3-(3-Chloro-4-fluorophenyl)-1-(8-fluoro-6-oxo-1,4,5,6-tetrahydro-2H-thiopyrano[3,4-c]isoquinolin-1-yl)-1-methylurea;
(S)-3-(3-Chloro-4-fluorophenyl)-1-(8-fluoro-6-oxo-1,4,5,6-tetrahydro-2H-thiopyrano[3,4-c]isoquinolin-1-yl)-1-methylurea;
(R)-3-(3-Cyano-4-fluorophenyl)-1-(8-fluoro-6-oxo-1,4,5,6-tetrahydro-2H-thiopyrano[3,4-c]isoquinolin-1-yl)-1-methylurea;
(S)-3-(3-Cyano-4-fluorophenyl)-1-(8-fluoro-6-oxo-1,4,5,6-tetrahydro-2H-thiopyrano[3,4-c]isoquinolin-1-yl)-1-methylurea;
(R)-3-(3-Chloro-4-fluorophenyl)-1-(8,9-difluoro-6-oxo-1,4,5,6-tetrahydro-2H-thiopyrano[3,4-c]isoquinolin-1-yl)-1-methylurea;
(S)-3-(3-Chloro-4-fluorophenyl)-1-(8,9-difluoro-6-oxo-1,4,5,6-tetrahydro-2H-thiopyrano[3,4-c]isoquinolin-1-yl)-1-methylurea;
(R)-3-(3-Cyano-4-fluorophenyl)-1-(8,9-difluoro-6-oxo-1,4,5,6-tetrahydro-2H-thiopyrano[3,4-c]isoquinolin-1-yl)-1-methylurea;
(S)-3-(3-Cyano-4-fluorophenyl)-1-(8,9-difluoro-6-oxo-1,4,5,6-tetrahydro-2H-thiopyrano[3,4-c]isoquinolin-1-yl)-1-methylurea;
(R)-1-(8,9-Difluoro-6-oxo-1,2,3,4,5,6-hexahydrobenzo[c][1,7]naphthyridin-1-yl)-3-(3,4-difluorophenyl)-1-methylurea;
(S)-1-(8,9-Difluoro-6-oxo-1,2,3,4,5,6-hexahydrobenzo[c][1,7]naphthyridin-1-yl)-3-(3,4-difluorophenyl)-1-methylurea;
3-(3-Chloro-4-fluorophenyl)-(1R)-(8-fluoro-3R-oxido-6-oxo-1,4,5,6-tetrahydro-2H-thiopyrano[3,4-c]isoquinolin-1-yl)-1-methylurea;
3-(3-Chloro-4-fluorophenyl)-(1R)-(8-fluoro-3 S-oxido-6-oxo-1,4,5,6-tetrahydro-2H-thiopyrano[3,4-c]isoquinolin-1-yl)-1-methylurea;
3-(3-Chloro-4-fluorophenyl)-(1S)-(8-fluoro-3R-oxido-6-oxo-1,4,5,6-tetrahydro-2H-thiopyrano[3,4-c]isoquinolin-1-yl)-1-methylurea;
3-(3-Chloro-4-fluorophenyl)-(1S)-(8-fluoro-3S-oxido-6-oxo-1,4,5,6-tetrahydro-2H-thiopyrano[3,4-c]isoquinolin-1-yl)-1-methylurea;
(R)-3-(3-Chloro-4-fluorophenyl)-1-(8-fluoro-3,3-dioxido-6-oxo-1,4,5,6-tetrahydro-2H-thiopyrano[3,4-c]isoquinolin-1-yl)-1-methylurea;
(S)-3-(3-Chloro-4-fluorophenyl)-1-(8-fluoro-3,3-dioxido-6-oxo-1,4,5,6-tetrahydro-2H-thiopyrano[3,4-c]isoquinolin-1-yl)-1-methylurea;
3-(3-Cyano-4-fluorophenyl)-(1R)-(8-fluoro-3R-oxido-6-oxo-1,4,5,6-tetrahydro-2H-thiopyrano[3,4-c]isoquinolin-1-yl)-1-methylurea;
3-(3-Cyano-4-fluorophenyl)-(1R)-(8-fluoro-3 S-oxido-6-oxo-1,4,5,6-tetrahydro-2H-thiopyrano[3,4-c]isoquinolin-1-yl)-1-methylurea;
3-(3-Cyano-4-fluorophenyl)-(1S)-(8-fluoro-3R-oxido-6-oxo-1,4,5,6-tetrahydro-2H-thiopyrano[3,4-c]isoquinolin-1-yl)-1-methylurea;
3-(3-Cyano-4-fluorophenyl)-(1S)-(8-fluoro-3S-oxido-6-oxo-1,4,5,6-tetrahydro-2H-thiopyrano[3,4-c]isoquinolin-1-yl)-1-methylurea;
(R)-3-(3-Chloro-4-fluorophenyl)-1-(8,9-difluoro-3,3-dioxido-6-oxo-1,4,5,6-tetrahydro-2H-thiopyrano[3,4-c]isoquinolin-1-yl)-1-methylurea;
(S)-3-(3-Chloro-4-fluorophenyl)-1-(8,9-difluoro-3,3-dioxido-6-oxo-1,4,5,6-tetrahydro-2H-thiopyrano[3,4-c]isoquinolin-1-yl)-1-methylurea;
(R)-3-(3-Cyano-4-fluorophenyl)-1-(8,9-difluoro-3,3-dioxido-6-oxo-1,4,5,6-tetrahydro-2H-thiopyrano[3,4-c]isoquinolin-1-yl)-1-methylurea;
(S)-3-(3-Cyano-4-fluorophenyl)-1-(8,9-difluoro-3,3-dioxido-6-oxo-1,4,5,6-tetrahydro-2H-thiopyrano[3,4-c]isoquinolin-1-yl)-1-methylurea;
3-(3-Chloro-4-fluorophenyl)-(1R)-(8,9-difluoro-3R-oxido-6-oxo-1,4,5,6-tetrahydro-2H-thiopyrano[3,4-c]isoquinolin-1-yl)-1-methylurea;
3-(3-Chloro-4-fluorophenyl)-(1R)-(8,9-difluoro-3 S-oxido-6-oxo-1,4,5,6-tetrahydro-2H-thiopyrano[3,4-c]isoquinolin-1-yl)-1-methylurea;
3-(3-Chloro-4-fluorophenyl)-(1S)-(8,9-difluoro-3R-oxido-6-oxo-1,4,5,6-tetrahydro-2H-thiopyrano[3,4-c]isoquinolin-1-yl)-1-methylurea;
3-(3-Chloro-4-fluorophenyl)-(1S)-(8,9-difluoro-3S-oxido-6-oxo-1,4,5,6-tetrahydro-2H-thiopyrano[3,4-c]isoquinolin-1-yl)-1-methylurea;
3-(3-Cyano-4-fluorophenyl)-(1R)-(8,9-difluoro-3R-oxido-6-oxo-1,4,5,6-tetrahydro-2H-thiopyrano[3,4-c]isoquinolin-1-yl)-1-methylurea;
3-(3-Cyano-4-fluorophenyl)-(1R)-(8,9-difluoro-3 S-oxido-6-oxo-1,4,5,6-tetrahydro-2H-thiopyrano[3,4-c]isoquinolin-1-yl)-1-methylurea;
3-(3-Cyano-4-fluorophenyl)-(1S)-(8,9-difluoro-3R-oxido-6-oxo-1,4,5,6-tetrahydro-2H-thiopyrano[3,4-c]isoquinolin-1-yl)-1-methylurea;
3-(3-Cyano-4-fluorophenyl)-(1S)-(8,9-difluoro-3S-oxido-6-oxo-1,4,5,6-tetrahydro-2H-thiopyrano[3,4-c]isoquinolin-1-yl)-1-methylurea;
(R)-3-(3-Cyano-4-fluorophenyl)-1-(8-fluoro-3,3-dioxido-6-oxo-1,4,5,6-tetrahydro-2H-thiopyrano[3,4-c]isoquinolin-1-yl)-1-methylurea;
(S)-3-(3-Cyano-4-fluorophenyl)-1-(8-fluoro-3,3-dioxido-6-oxo-1,4,5,6-tetrahydro-2H-thiopyrano[3,4-c]isoquinolin-1-yl)-1-methylurea;
(R)-1-(8,9-difluoro-6-oxo-1,2,3,4,5,6-hexahydrobenzo[c][1,7]naphthyridin-1-yl)-3-(3-(difluoromethyl)-4-fluorophenyl)-1-methylurea;
(S)-1-(8,9-difluoro-6-oxo-1,2,3,4,5,6-hexahydrobenzo[c][1,7]naphthyridin-1-yl)-3-(3-(difluoromethyl)-4-fluorophenyl)-1-methylurea;
(R)—N-(8,9-difluoro-6-oxo-1,4,5,6-tetrahydro-2H-pyrano[3,4-c]isoquinolin-1-yl)-N-methylisoindoline-2-carboxamide;
(S)—N-(8,9-difluoro-6-oxo-1,4,5,6-tetrahydro-2H-pyrano[3,4-c]isoquinolin-1-yl)-N-methylisoindoline-2-carboxamide;
(R)-5-chloro-N-(8,9-difluoro-6-oxo-1,4,5,6-tetrahydro-2H-pyrano[3,4-c]isoquinolin-1-yl)-N-methylisoindoline-2-carboxamide;
(S)-5-chloro-N-(8,9-difluoro-6-oxo-1,4,5,6-tetrahydro-2H-pyrano[3,4-c]isoquinolin-1-yl)-N-methylisoindoline-2-carboxamide;
(R)-5-bromo-N-(8,9-difluoro-6-oxo-1,4,5,6-tetrahydro-2H-pyrano[3,4-c]isoquinolin-1-yl)-N-methylisoindoline-2-carboxamide;
(S)-5-bromo-N-(8,9-difluoro-6-oxo-1,4,5,6-tetrahydro-2H-pyrano[3,4-c]isoquinolin-1-yl)-N-methylisoindoline-2-carboxamide;
(R)-5-fluoro-N-(8,9-difluoro-6-oxo-1,4,5,6-tetrahydro-2H-pyrano[3,4-c]isoquinolin-1-yl)-N-methylisoindoline-2-carboxamide;
(S)-5-fluoro-N-(8,9-difluoro-6-oxo-1,4,5,6-tetrahydro-2H-pyrano[3,4-c]isoquinolin-1-yl)-N-methylisoindoline-2-carboxamide;
(R)—N-(8,9-difluoro-6-oxo-1,2,3,4,5,6-hexahydrobenzo[c][1,7]naphthyridin-1-yl)-N-methylisoindoline-2-carboxamide;
(S)—N-(8,9-difluoro-6-oxo-1,2,3,4,5,6-hexahydrobenzo[c][1,7]naphthyridin-1-yl)-N-methylisoindoline-2-carboxamide;
(R)—N-(8,9-difluoro-6-oxo-1,2,3,4,5,6-hexahydrobenzo[c][1,7]naphthyridin-1-yl)-5-fluoro-N-methylisoindoline-2-carboxamide;
(S)—N-(8,9-difluoro-6-oxo-1,2,3,4,5,6-hexahydrobenzo[c][1,7]naphthyridin-1-yl)-5-fluoro-N-methylisoindoline-2-carboxamide;
(R)—N-(8,9-difluoro-6-oxo-1,2,3,4,5,6-hexahydrobenzo[c][1,7]naphthyridin-1-yl)-5-chloro-N-methylisoindoline-2-carboxamide;
(S)—N-(8,9-difluoro-6-oxo-1,2,3,4,5,6-hexahydrobenzo[c][1,7]naphthyridin-1-yl)-5-chloro-N-methylisoindoline-2-carboxamide;
(R)—N-(8,9-difluoro-6-oxo-1,2,3,4,5,6-hexahydrobenzo[c][1,7]naphthyridin-1-yl)-5-bromo-N-methylisoindoline-2-carboxamide;
(S)—N-(8,9-difluoro-6-oxo-1,2,3,4,5,6-hexahydrobenzo[c][1,7]naphthyridin-1-yl)-5-bromo-N-methylisoindoline-2-carboxamide;
(R)—N-(8,9-difluoro-6-oxo-1,4,5,6-tetrahydro-2H-pyrano[3,4-c]isoquinolin-1-yl)-N-methyl-5-(trifluoromethyl)isoindoline-2-carboxamide;
(S)—N-(8,9-difluoro-6-oxo-1,4,5,6-tetrahydro-2H-pyrano[3,4-c]isoquinolin-1-yl)-N-methyl-5-(trifluoromethyl)isoindoline-2-carboxamide;
(R)—N-(8,9-difluoro-6-oxo-1,2,3,4,5,6-hexahydrobenzo[c][1,7]naphthyridin-1-yl)-N-methyl-5-(trifluoromethyl)isoindoline-2-carboxamide;
(S)—N-(8,9-difluoro-6-oxo-1,2,3,4,5,6-hexahydrobenzo[c][1,7]naphthyridin-1-yl)-N-methyl-5-(trifluoromethyl)isoindoline-2-carboxamide;
(R)-1-(8,9-difluoro-5-methyl-6-oxo-1,4,5,6-tetrahydro-2H-pyrano[3,4-c]isoquinolin-1-yl)-3-(3-(difluoromethyl)-4-fluorophenyl)-1-methylurea;
(S)-1-(8,9-difluoro-5-methyl-6-oxo-1,4,5,6-tetrahydro-2H-pyrano[3,4-c]isoquinolin-1-yl)-3-(3-(difluoromethyl)-4-fluorophenyl)-1-methylurea;
(R)-1-(8,9-difluoro-6-methoxy-1,4-dihydro-2H-pyrano[3,4-c]isoquinolin-1-yl)-3-(3-(difluoromethyl)-4-fluorophenyl)-1-methylurea;
(S)-1-(8,9-difluoro-6-methoxy-1,4-dihydro-2H-pyrano[3,4-c]isoquinolin-1-yl)-3-(3-(difluoromethyl)-4-fluorophenyl)-1-methylurea;
(R)-1-(8,9-difluoro-6-(methylamino)-1,4-dihydro-2H-pyrano[3,4-c]isoquinolin-1-yl)-3-(3-(difluoromethyl)-4-fluorophenyl)-1-methylurea
(S)-1-(8,9-difluoro-6-(methylamino)-1,4-dihydro-2H-pyrano[3,4-c]isoquinolin-1-yl)-3-(3-(difluoromethyl)-4-fluorophenyl)-1-methylurea;
(R)-1-(8,9-difluoro-6-((2-hydroxyethyl)amino)-1,4-dihydro-2H-pyrano[3,4-c]isoquinolin-1-yl)-3-(3-(difluoromethyl)-4-fluorophenyl)-1-methylurea;
(S)-1-(8,9-difluoro-6-((2-hydroxyethyl)amino)-1,4-dihydro-2H-pyrano[3,4-c]isoquinolin-1-yl)-3-(3-(difluoromethyl)-4-fluorophenyl)-1-methylurea;
(R)-1-(8,9-difluoro-6-((2-aminoethyl)amino)-1,4-dihydro-2H-pyrano[3,4-c]isoquinolin-1-yl)-3-(3-(difluoromethyl)-4-fluorophenyl)-1-methylurea
(S)-1-(8,9-difluoro-6-((2-aminoethyl)amino)-1,4-dihydro-2H-pyrano[3,4-c]isoquinolin-1-yl)-3-(3-(difluoromethyl)-4-fluorophenyl)-1-methylurea;
(R)-3-(3-chloro-4-fluorophenyl)-1-(8,9-difluoro-6-((2-aminoethyl)amino)-1,4-dihydro-2H-pyrano[3,4-c]isoquinolin-1-yl)-1-methylurea;
(S)-3-(3-chloro-4-fluorophenyl)-1-(8,9-difluoro-6-((2-aminoethyl)amino)-1,4-dihydro-2H-pyrano[3,4-c]isoquinolin-1-yl)-1-methylurea;
(R)-3-(3-chloro-4-fluorophenyl)-1-(8,9-difluoro-6-(methylamino)-1,4-dihydro-2H-pyrano[3,4-c]isoquinolin-1-yl)-1-methylurea;
(S)-3-(3-chloro-4-fluorophenyl)-1-(8,9-difluoro-6-(methylamino)-1,4-dihydro-2H-pyrano[3,4-c]isoquinolin-1-yl)-1-methylurea;
(R)-1-(8,9-difluoro-5-methyl-6-oxo-1,4,5,6-tetrahydro-2H-pyrano[3,4-c]isoquinolin-1-yl)-3-(3-chloro-4-fluorophenyl)-1-methylurea;
(S)-1-(8,9-difluoro-5-methyl-6-oxo-1,4,5,6-tetrahydro-2H-pyrano[3,4-c]isoquinolin-1-yl)-3-(3-chloro-4-fluorophenyl)-1-methylurea;
(R)-3-(3-chloro-4-fluorophenyl)-1-(8,9-difluoro-6-methoxy-1,4-dihydro-2H-pyrano[3,4-c]isoquinolin-1-yl)-1-methylurea;
(S)-3-(3-chloro-4-fluorophenyl)-1-(8,9-difluoro-6-methoxy-1,4-dihydro-2H-pyrano[3,4-c]isoquinolin-1-yl)-1-methylurea;
(R)-3-(3-chloro-4-fluorophenyl)-1-(8,9-difluoro-6-((2-hydroxyethyl)amino)-1,4-dihydro-2H-pyrano[3,4-c]isoquinolin-1-yl)-1-methylurea;
(S)-3-(3-chloro-4-fluorophenyl)-1-(8,9-difluoro-6-((2-hydroxyethyl)amino)-1,4-dihydro-2H-pyrano[3,4-c]isoquinolin-1-yl)-1-methylurea;
(R)-1-(8,9-difluoro-5-(2-hydroxyethyl)-6-oxo-1,4,5,6-tetrahydro-2H-pyrano[3,4-c]isoquinolin-1-yl)-3-(3-(difluoromethyl)-4-fluorophenyl)-1-methylurea;
(S)-1-(8,9-difluoro-5-(2-hydroxyethyl)-6-oxo-1,4,5,6-tetrahydro-2H-pyrano[3,4-c]isoquinolin-1-yl)-3-(3-(difluoromethyl)-4-fluorophenyl)-1-methylurea;
(R)-1-(6-(2-aminoethoxy)-8,9-difluoro-1,4-dihydro-2H-pyrano[3,4-c]isoquinolin-1-yl)-3-(3-(difluoromethyl)-4-fluorophenyl)-1-methylurea;
(S)-1-(6-(2-aminoethoxy)-8,9-difluoro-1,4-dihydro-2H-pyrano[3,4-c]isoquinolin-1-yl)-3-(3-(difluoromethyl)-4-fluorophenyl)-1-methylurea;
(R)-1-(5-(2-aminoethyl)-8,9-difluoro-6-oxo-1,4,5,6-tetrahydro-2H-pyrano[3,4-c]isoquinolin-1-yl)-3-(3-(difluoromethyl)-4-fluorophenyl)-1-methylurea;
(S)-1-(5-(2-aminoethyl)-8,9-difluoro-6-oxo-1,4,5,6-tetrahydro-2H-pyrano[3,4-c]isoquinolin-1-yl)-3-(3-(difluoromethyl)-4-fluorophenyl)-1-methylurea;
(R)-3-(3-chloro-4-fluorophenyl)-1-(8,9-difluoro-5-(2-hydroxyethyl)-6-oxo-1,4,5,6-tetrahydro-2H-pyrano[3,4-c]isoquinolin-1-yl)-1-methylurea;
(S)-3-(3-chloro-4-fluorophenyl)-1-(8,9-difluoro-5-(2-hydroxyethyl)-6-oxo-1,4,5,6-tetrahydro-2H-pyrano[3,4-c]isoquinolin-1-yl)-1-methylurea;
(R)-3-(3-chloro-4-fluorophenyl)-1-(8,9-difluoro-6-(2-hydroxyethoxy)-1,4-dihydro-2H-pyrano[3,4-c]isoquinolin-1-yl)-1-methylurea;
(S)-3-(3-chloro-4-fluorophenyl)-1-(8,9-difluoro-6-(2-hydroxyethoxy)-1,4-dihydro-2H-pyrano[3,4-c]isoquinolin-1-yl)-1-methylurea;
(R)-1-(8,9-difluoro-6-(2-hydroxyethoxy)-1,4-dihydro-2H-pyrano[3,4-c]isoquinolin-1-yl)-3-(3-(difluoromethyl)-4-fluorophenyl)-1-methylurea;
(S)-1-(8,9-difluoro-6-(2-hydroxyethoxy)-1,4-dihydro-2H-pyrano[3,4-c]isoquinolin-1-yl)-3-(3-(difluoromethyl)-4-fluorophenyl)-1-methylurea;
(R)-1-(5-(2-aminoethyl)-8,9-difluoro-6-oxo-1,4,5,6-tetrahydro-2H-pyrano[3,4-c]isoquinolin-1-yl)-3-(3-chloro-4-fluorophenyl)-1-methylurea;
(S)-1-(5-(2-aminoethyl)-8,9-difluoro-6-oxo-1,4,5,6-tetrahydro-2H-pyrano[3,4-c]isoquinolin-1-yl)-3-(3-chloro-4-fluorophenyl)-1-methylurea;
(R)-1-(6-(2-aminoethoxy)-8,9-difluoro-1,4-dihydro-2H-pyrano[3,4-c]isoquinolin-1-yl)-3-(3-chloro-4-fluorophenyl)-1-methylurea;
(S)-1-(6-(2-aminoethoxy)-8,9-difluoro-1,4-dihydro-2H-pyrano[3,4-c]isoquinolin-1-yl)-3-(3-chloro-4-fluorophenyl)-1-methylurea;
or a salt, solvate, prodrug, isotopically labelled, stereoisomer, any mixture of stereoisomers, tautomer, and/or any mixture of tautomers thereof.

Embodiment 20 provides a pharmaceutical composition comprising at least one compound of any one of Embodiments 1-19 and a pharmaceutically acceptable carrier.

Embodiment 21 provides the pharmaceutical composition of Embodiment 20, further comprising at least one additional agent useful for treating hepatitis infection.

Embodiment 22 provides the pharmaceutical composition of Embodiment 21, wherein the at least one additional agent comprises at least one selected from the group consisting of reverse transcriptase inhibitor; capsid inhibitor; cccDNA formation inhibitor; RNA destabilizer; oligomeric nucleotide targeted against the HBV genome; immunostimulator; and GalNAc-siRNA conjugate targeted against an HBV gene transcript.

Embodiment 23 provides the pharmaceutical composition of Embodiment 22, wherein the immunostimulator is a checkpoint inhibitor.

Embodiment 24 provides the pharmaceutical composition of Embodiment 23, wherein the checkpoint inhibitor is a PD-L1 inhibitor.

Embodiment 25 provides a method of treating, ameliorating, and/or preventing hepatitis B virus (HBV) infection in a subject, the method comprising administering to the subject in need thereof a therapeutically effective amount of at least one compound of any one of Embodiments 1-19 and/or at least one pharmaceutical composition of any one of Embodiments 20-24.

Embodiment 26 provides the method of Embodiment 25, wherein the subject is further infected with hepatitis D virus (HDV).

Embodiment 27 provides the method of any one of Embodiments 25-26, wherein the at least one compound and/or composition is administered to the subject in a pharmaceutically acceptable composition.

Embodiment 28 provides the method of any one of Embodiments 25-27, wherein the subject is further administered at least one additional agent useful for treating, ameliorating, and/or preventing the hepatitis B virus infection.

Embodiment 29 provides the method of Embodiment 28, wherein the at least one additional agent comprises at least one selected from the group consisting of reverse transcriptase inhibitor; capsid inhibitor; cccDNA formation inhibitor; RNA destabilizer; oligomeric nucleotide targeted against the HBV genome; immunostimulator; and GalNAc-siRNA conjugate targeted against an HBV gene transcript.

Embodiment 30 provides the method of Embodiment 29, wherein the immunostimulator is a checkpoint inhibitor.

Embodiment 31 provides the method of Embodiment 30, wherein the checkpoint inhibitor is a PD-L1 inhibitor.

Embodiment 32 provides the method of any one of Embodiments 28-31, wherein the subject is co-administered the at least one compound and/or composition and the at least one additional agent.

Embodiment 33 provides the method of any one of Embodiments 28-32, wherein the at least one compound and/or composition and the at least one additional agent are coformulated.

Embodiment 34 provides a method of inhibiting expression and/or function of a viral capsid protein directly or indirectly in a hepatis B virus-infected subject, the method comprising administering to the subject in need thereof a therapeutically effective amount of at least one compound of any one of Embodiments 1-19 and/or at least one pharmaceutical composition of any one of Embodiments 20-24.

Embodiment 35 provides the method of Embodiment 34, wherein the subject is further infected with hepatitis D virus (HDV).

Embodiment 36 provides the method of any one of Embodiments 34-35, wherein the at least one compound and/or composition is administered to the subject in a pharmaceutically acceptable composition.

Embodiment 37 provides the method of any one of Embodiments 34-36, wherein the subject is further administered at least one additional agent useful for treating, ameliorating, and/or preventing the hepatitis B viral infection.

Embodiment 38 provides the method of Embodiment 37, wherein the at least one additional agent comprises at least one selected from the group consisting of reverse transcriptase inhibitor; capsid inhibitor; cccDNA formation inhibitor; RNA destabilizer; oligomeric nucleotide targeted against the HBV genome; immunostimulator; and GalNAc-siRNA conjugate targeted against an HBV gene transcript.

Embodiment 39 provides the method of Embodiment 38, wherein the immunostimulator is a checkpoint inhibitor.

Embodiment 40 provides the method of Embodiment 39, wherein the checkpoint inhibitor is a PD-L1 inhibitor.

Embodiment 41 provides the method of any one of Embodiments 37-40, wherein the subject is co-administered the at least one compound and/or composition and the at least one additional agent.

Embodiment 42 provides the method of any one of Embodiments 37-41, wherein the at least one compound and/or composition and the at least one additional agent are coformulated.

Embodiment 43 provides the method of any one of Embodiments 25-42, wherein the subject is a mammal.

Embodiment 44 provides the method of Embodiment 43, wherein the mammal is a human.

The disclosures of each and every patent, patent application, and publication cited herein are hereby incorporated herein by reference in their entirety. While this disclosure has been disclosed with reference to specific embodiments, it is apparent that other embodiments and variations of this disclosure may be devised by others skilled in the art without departing from the true spirit and scope of the disclosure. The appended claims are intended to be construed to include all such embodiments and equivalent variations.

1. A compound of formula (I), or a salt, solvate, prodrug, stereoisomer, tautomer, or isotopically labelled derivative thereof, or any mixtures thereof: 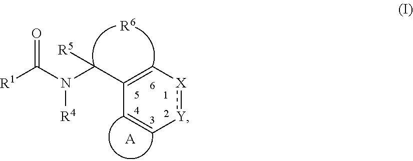
wherein:
X, Y, and the bond between X and Y are such that:
X is NR⁷, Y is C(═O), and the bond between X and Y is a single bond, or
X is N, Y is CR¹⁰, and the bond between X and Y is a double bond;
A ring 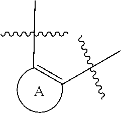
is selected from the group consisting of: 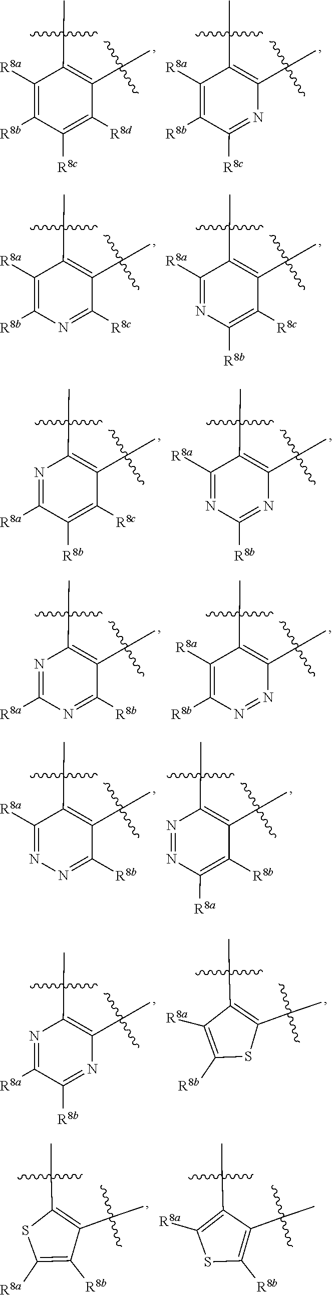
wherein there is no bridgehead double bond), 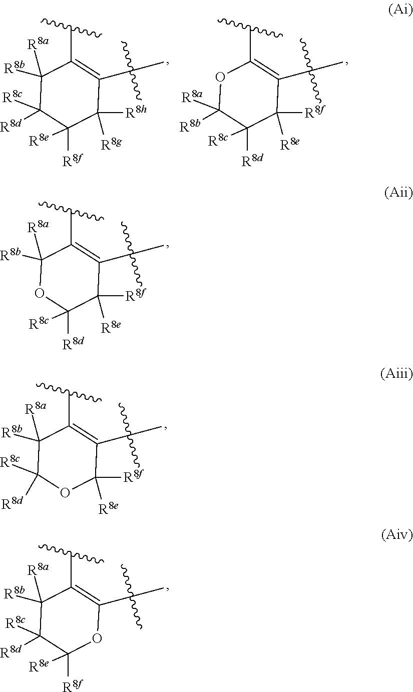
wherein:
in (Ai) R^{8a} and R^{8b} optionally combine with the carbon atom to which they are attached to form carbonyl (—(C═O)—);
in (Aii) R^{8a} and R^{8b}, or R^{8c} and R^{8d}, optionally combine with the carbon atom to which they are attached to form carbonyl (—(C═O)—);
in (Aiii) R^{8c} and R^{8d}, or R^{8e} and R^{8f}, optionally combine with the carbon atom to which they are attached to form carbonyl (—(C═O)—);
in (Aiv) R^{8e} and R^{8f} optionally combine with the carbon atom to which they are attached to form carbonyl (—(C═O)—);
or the A ring is absent, position 3 of the pyridin-2-one ring is substituted with R^{8a}, and position 4 of the pyridin-2-one ring is substituted with R^{8b};
R¹ is —NR²R³ or optionally substituted isoindolin-2-yl;
R² is selected from the group consisting of optionally substituted C₃-C₈ cycloalkyl, optionally substituted phenyl, optionally substituted benzyl, optionally substituted heteroaryl, and —(CH₂)(optionally substituted heteroaryl);
R³ is selected from the group consisting of H and C₁-C₆ alkyl;
R⁴ is selected from the group consisting of H, C₁-C₆ alkyl, and C₃-C₈ cycloalkyl, wherein the alkyl or cycloalkyl is optionally substituted with at least one selected from the group consisting of C₁-C₆ alkyl, C₃-C₈ cycloalkyl, halogen, cyano, —OH, C₁-C₆ alkoxy, C₃-C₈ cycloalkoxy, C₁-C₆ haloalkoxy, C₃-C₈ halocycloalkoxy, optionally substituted phenyl, optionally substituted heteroaryl, optionally substituted heterocyclyl, —C(═O)OR⁹, —OC(═O)R⁹, —SR⁹, —S(═O)R⁹, —S(═O)₂R⁹, —S(═O)₂NR⁹R⁹, —N(R⁹)S(═O)₂R⁹, —N(R⁹)C(═O)R⁹, —C(═O)NR⁹R⁹, and —NR⁹R⁹;
R⁵ is selected from the group consisting of H and optionally substituted C₁-C₆ alkyl;
R⁶ is —(CH₂)ₚ-Q-(CH₂)_{q}—,
wherein p and q are independently 0, 1, 2, or 3, and
Q is a bond (absent), —O—, —OCH(OH)—, —CH(OH)O—, —S—, —S(═O)—, —S(═O)₂—, —NR¹¹, —CH(OH)—, —C(═O)—, —C(═O)O—, or —OC(═O)—,
wherein p and q are selected such that:
2≤(p+q)≤4 if Q is a bond,
1≤(p+q)≤3 if Q is —O—, S—, —S(═O)—, —S(═O)₂—, —NR¹¹, —CH(OH)—, or —C(═O)—,
0≤(p+q)≤2 if Q is —C(═O)O—, —OC(═O)—, —OCH(OH)—, or —CH(OH)O—, and
wherein each CH₂ is optionally independently substituted with one or two methyl groups;
R⁷ is selected from the group consisting of H, optionally substituted C₁-C₆ alkyl, and optionally substituted C₃-C₈ cycloalkyl;
each occurrence of R^{8a}, R^{8b}, R^{8c}, R^{8d}, R^{8e}, R^{8f}, R^{8g}, and R^{8h} is independently selected from the group consisting of H, halogen, cyano, optionally substituted C₁-C₆ alkyl, optionally substituted C₃-C₈ cycloalkyl, optionally substituted C₁-C₆ alkoxy, optionally substituted C₃-C₈ cycloalkoxy, heterocyclyl, heteroaryl, —S(optionally substituted C₁-C₆ alkyl), —SO(optionally substituted C₁-C₆ alkyl), —SO₂(optionally substituted C₁-C₆ alkoxy), —C(═O)OH, —C(═O)O(optionally substituted C₁-C₆ alkyl), —C(═O)O(optionally substituted C₃-C₈ cycloalkyl), —O(optionally substituted C₁-C₆ alkyl), —O(optionally substituted C₃-C₈ cycloalkyl), —NH₂, —NH(optionally substituted C₁-C₆ alkyl), —NH(optionally substituted C₃-C₈ cycloalkyl), —N(optionally substituted C₁-C₆ alkyl)(optionally substituted C₁-C₆ alkyl), —N(optionally substituted C₃-C₈ cycloalkyl)(optionally substituted C₃-C₈ cycloalkyl), —N(optionally substituted C₁-C₆ alkyl)(optionally substituted C₃-C₈ cycloalkyl), —C(═O)NH₂, —C(═O)NH(optionally substituted C₁-C₆ alkyl), —C(═O)NH(optionally substituted C₃-C₈ cycloalkyl), —C(═O)N(optionally substituted C₁-C₆ alkyl)(optionally substituted C₁-C₆ alkyl), —C(═O)N(optionally substituted C₃-C₈ cycloalkyl)(optionally substituted C₃-C₈ cycloalkyl), and —C(═O)N(optionally substituted C₁-C₆ alkyl)(optionally substituted C₃-C₈ cycloalkyl;
each occurrence of R⁹ is independently selected from the group consisting of H, optionally substituted C₁-C₆ alkyl, optionally substituted C₃-C₈ cycloalkyl, optionally substituted phenyl, and optionally substituted hetereoaryl;
R¹⁰ is selected from the group consisting of H, halogen, cyano, optionally substituted C₁-C₆ alkyl, optionally substituted C₃-C₈ cycloalkyl, optionally substituted C₁-C₆ alkoxy, optionally substituted C₃-C₈ cycloalkoxy, heterocyclyl, heteroaryl, —S(optionally substituted C₁-C₆ alkyl), —SO(optionally substituted C₁-C₆ alkyl), —SO₂(optionally substituted C₁-C₆ alkyl), —C(═O)OH, —C(═O)O(optionally substituted C₁-C₆ alkyl), —C(═O)O(optionally substituted C₃-C₈ cycloalkyl), —O(optionally substituted C₁-C₆ alkyl), —O(optionally substituted C₃-C₈ cycloalkyl), —NH₂, —NH(optionally substituted C₁-C₆ alkyl), —NH(optionally substituted C₃-C₈ cycloalkyl), —N(optionally substituted C₁-C₆ alkyl)(optionally substituted C₁-C₆ alkyl), —N(optionally substituted C₃-C₈ cycloalkyl)(optionally substituted C₃-C₈ cycloalkyl), —N(optionally substituted C₁-C₆ alkyl)(optionally substituted C₃-C₈ cycloalkyl), —C(═O)NH₂, —C(═O)NH(optionally substituted C₁-C₆ alkyl), —C(═O)NH(optionally substituted C₃-C₈ cycloalkyl), —C(═O)N(optionally substituted C₁-C₆ alkyl)(optionally substituted C₁-C₆ alkyl), —C(═O)N(optionally substituted C₃-C₈ cycloalkyl)(optionally substituted C₃-C₈ cycloalkyl), and —C(═O)N(optionally substituted C₁-C₆ alkyl)(optionally substituted C₃-C₈ cycloalkyl; and
R¹¹ is selected from the group consisting of H, optionally substituted C₁-C₆ alkyl, optionally substituted C₃-C₈ cycloalkyl, optionally substituted phenyl, optionally substituted heteroaryl, and optionally substituted C₁-C₆ acyl.

2. The compound of claim 1, which is:

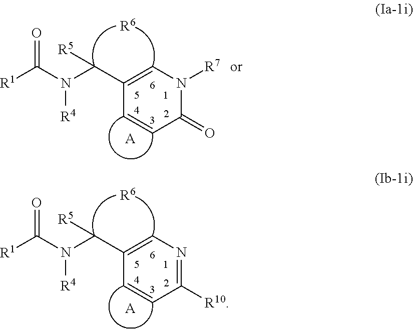

3. The compound of claim 1, wherein R⁵ is selected from the group consisting of H and CH₃.

4. The compound of claim 1, wherein each occurrence of aryl or heteroaryl is independently optionally substituted with at least one substituent selected from the group consisting of C₁-C₆ alkyl, C₃-C₈ cycloalkyl, phenyl, C₁-C₆ hydroxyalkyl, (C₁-C₆ alkoxy)-C₁-C₆ alkyl, C₁-C₆ haloalkyl, C₁-C₆ haloalkoxy, halogen, —CN, —OR^{b}, —N(R^{b})(R^{b}), —NO₂, —C(═O)N(R^{b})(R^{b}), —C(═O)OR^{b}, —OC(═O)R^{b}, —SR^{b}, —S(═O)R^{b}, —S(═O)₂R^{b}, N(R^{b})S(═O)₂R^{b}, —S(═O)₂N(R^{b})(R^{b}), acyl, and C₁-C₆ alkoxycarbonyl, wherein each occurrence of R^{b} is independently H, C₁-C₆ alkyl, or C₃-C₈ cycloalkyl, wherein in R^{b} the alkyl or cycloalkyl is optionally substituted with at least one selected from the group consisting of halogen, —OH, C₁-C₆ alkoxy, and heteroaryl; or substituents on two adjacent carbon atoms combine to form —O(CH₂)₁₋₃O—.

5. The compound of claim 1, wherein each occurrence of alkyl, alkenyl, alkynyl, or cycloalkyl is independently optionally substituted with at least one substituent selected from the group consisting of C₁-C₆ alkyl, C₃-C₈ cycloalkyl, halogen, cyano, —OR^{a}, optionally substituted phenyl, optionally substituted heteroaryl, optionally substituted heterocyclyl, —C(═O)OR^{a}, —OC(═O)R^{a}, —SR^{a}, —S(═O)R^{a}, —S(═O)₂R^{a}, —S(═O)₂NR^{a}R^{a}, —N(R^{a})S(═O)₂R^{a}, —N(R^{a})C(═O)R^{a}, —C(═O)NR^{a}R^{a}, and —N(R^{a})(R^{a}), wherein each occurrence of R^{a} is independently H, optionally substituted C₁-C₆ alkyl, optionally substituted C₃-C₈ cycloalkyl, optionally substituted aryl, or optionally substituted heteroaryl, or two R^{a} groups combine with the N to which they are bound to form a heterocycle.

6. The compound of claim 1, wherein R² is phenyl optionally substituted with at least one selected from the group consisting of C₁-C₆ alkyl, halogen, C₁-C₃ haloalkyl, and cyano.

7. The compound of claim 1, wherein R² is selected from the group consisting of phenyl, 3-chlorophenyl, 4-chlorophenyl, 3-fluorophenyl, 4-fluorophenyl, 3,4-difluorophenyl, 3,5-difluorophenyl, 2,4,5-trifluorophenyl, 3,4,5-trifluorophenyl, 3,4-dichlorophenyl, 3-chloro-4-fluorophenyl, 4-chloro-3-fluorophenyl, 4-chloro-3-methylphenyl, 3-chloro-4-methylphenyl, 4-fluoro-3-methylphenyl, 3-fluoro-4-methylphenyl, 4-chloro-3-methoxyphenyl, 3-chloro-4-methoxyphenyl, 4-fluoro-3-methoxyphenyl, 3-fluoro-4-methoxyphenyl, 3-trifluoromethylphenyl, 4-trifluoromethylphenyl, 3-trifluoromethyl-4-fluorophenyl, 4-trifluoromethyl-3-fluorophenyl, 3-cyanophenyl, 4-cyanophenyl, 3-cyano-4-fluorophenyl, 4-cyano-3-fluorophenyl, 3-difluoromethyl-4-fluorophenyl, and 4-difluoromethyl-3-fluorophenyl.

8. The compound of claim 1, wherein R³ is selected from the group consisting of H and methyl.

9. The compound of claim 1, wherein R⁶ is a divalent group selected from the group consisting of —CH₂CH₂—,—CH₂CH₂CH₂—, —CH₂OCH₂—, —CH₂OCH(OH)—, —CH(OH)OCH₂—, —CH₂OC(═O)—, —C(═O)OCH₂—, —CH₂SCH₂—, —CH₂S(═O)CH₂—, —CH₂S(═O)₂CH₂—, —CH₂NHCH₂—, —CH₂N(CH₃)CH₂—, —CH₂N[C(═O)CH₃]CH₂—, —CH₂N[CH₂CH₂OH]CH₂—, —CH₂CH₂CH₂CH₂—, —CH₂OCH₂CH₂—, and —CH₂CH₂OCH₂—, wherein each CH₂ group is optionally independently substituted with one or two CH₃ groups.

10. The compound of claim 1, which is selected from the group consisting of:

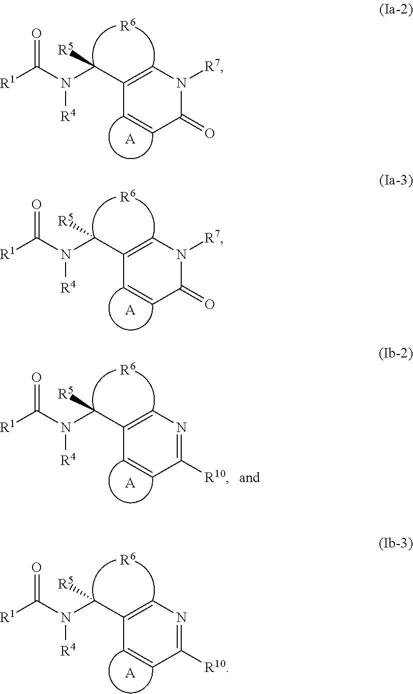

11. The compound of claim 1, which is selected from the group consisting of:

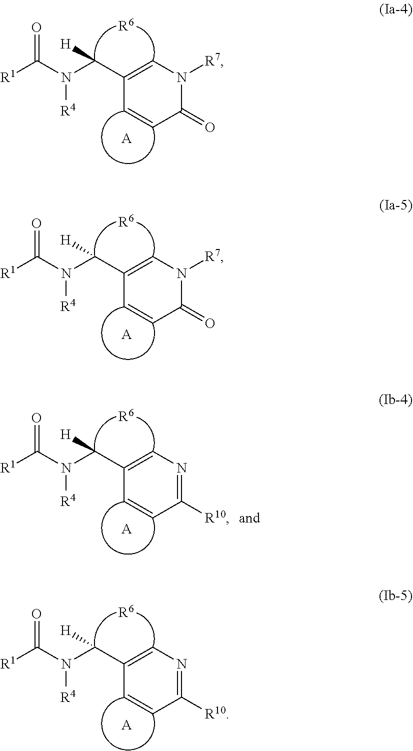

12. The compound of claim 1, which is selected from the group consisting of:

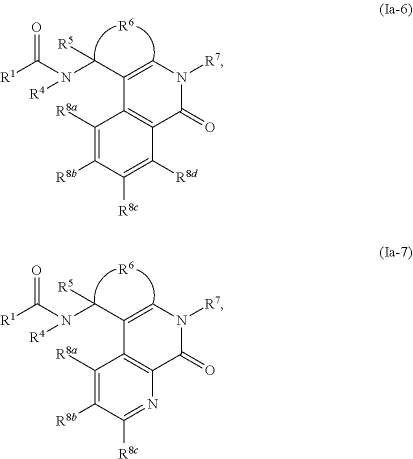

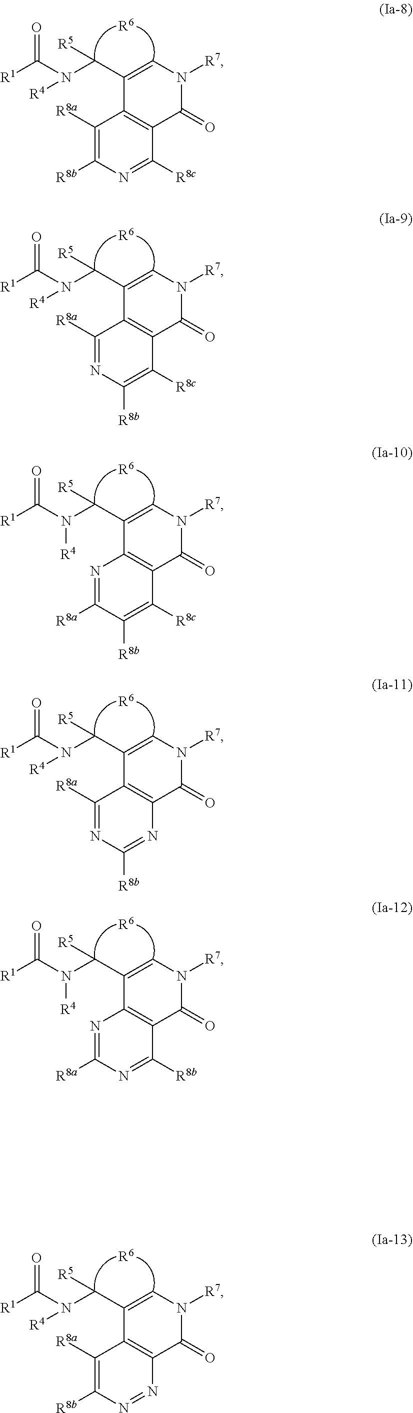

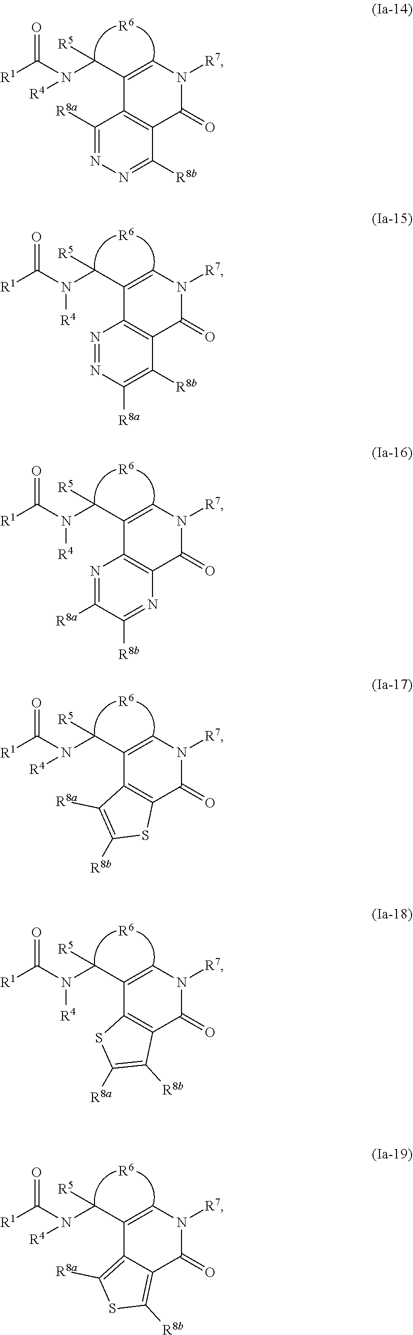

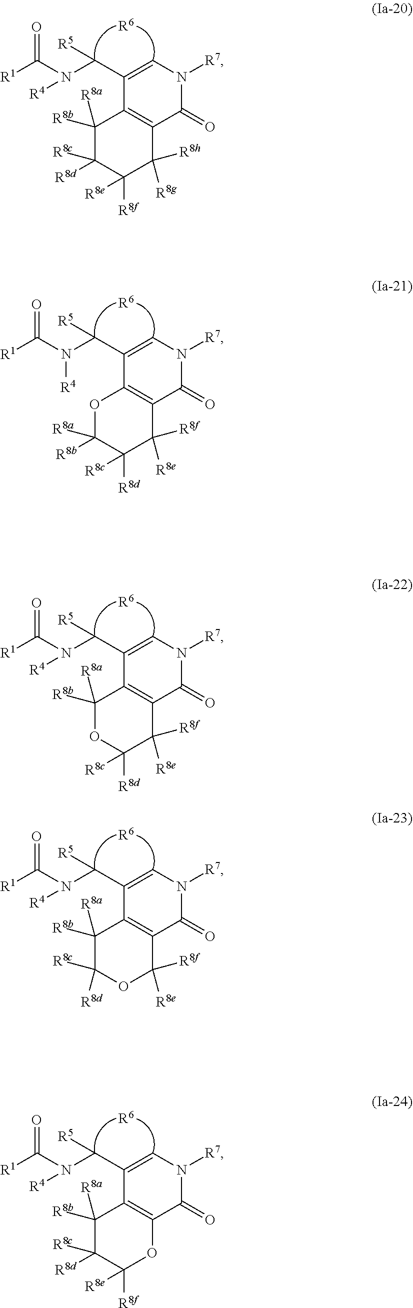

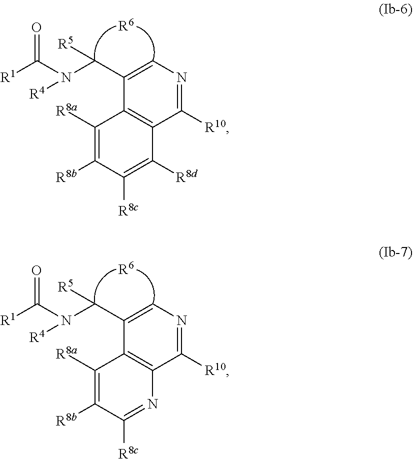

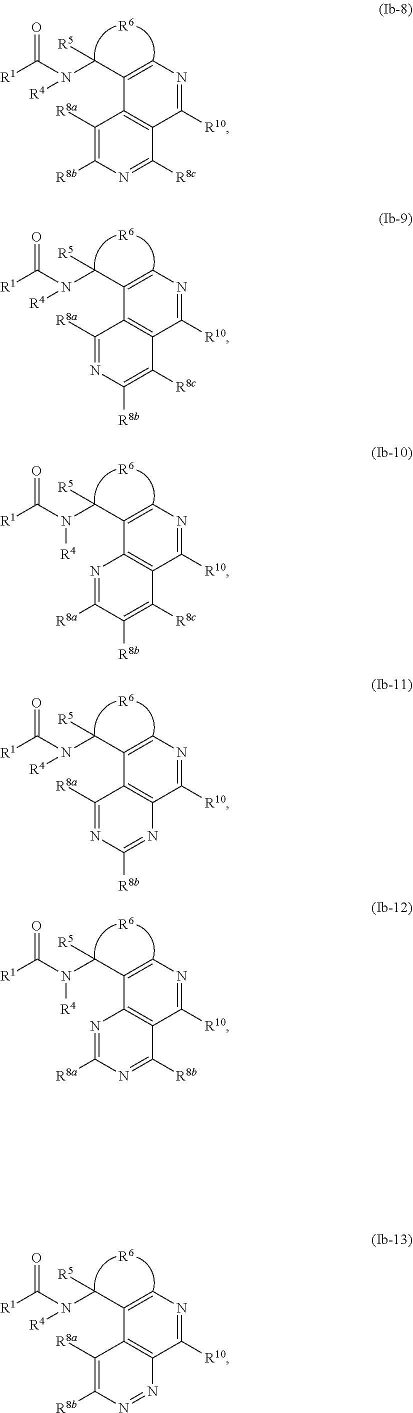

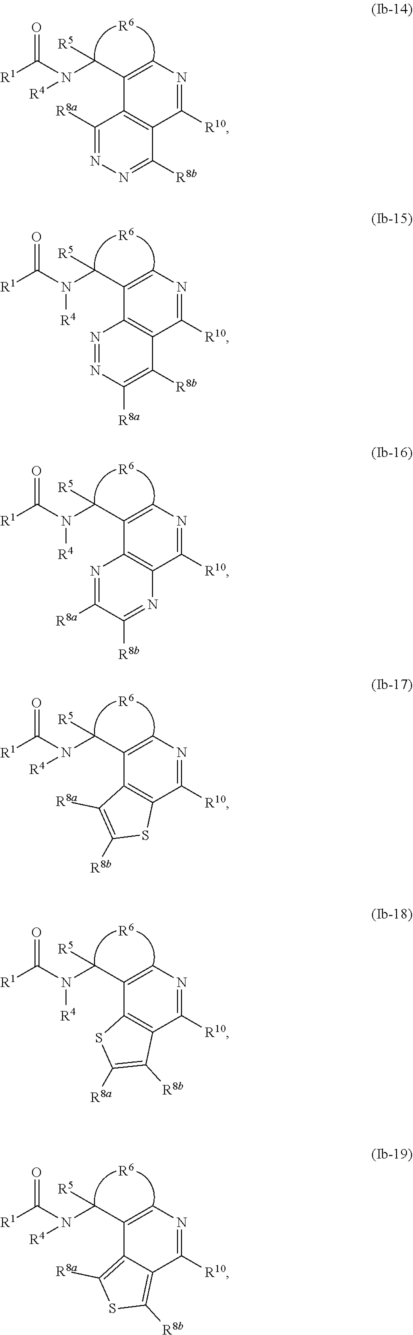

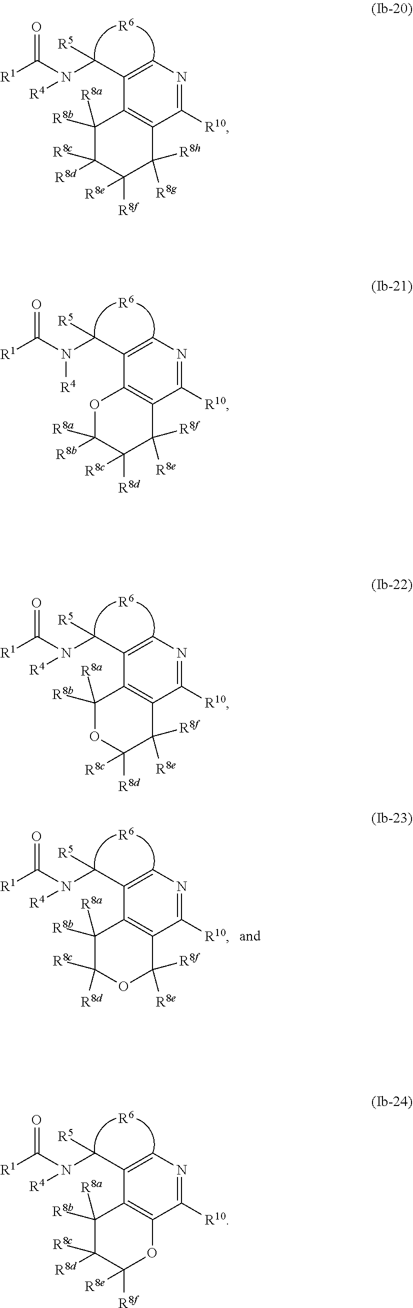

13. (canceled)

14. The compound of claim 1, which is selected from the group consisting of:

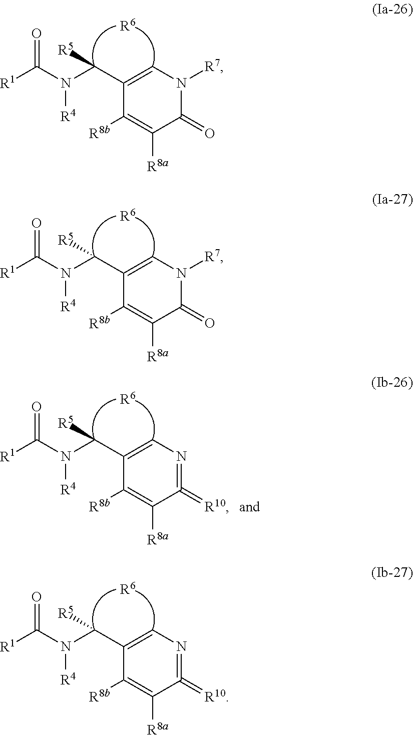

15. The compound of claim 1, which is selected from the group consisting of:

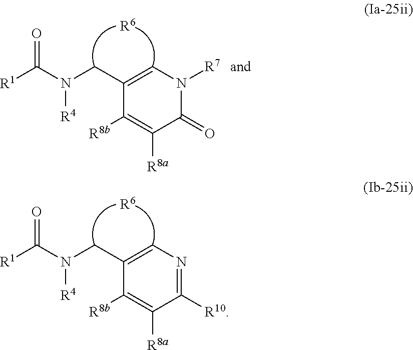

16. The compound of claim 1, which is at least one selected from the group consisting of:

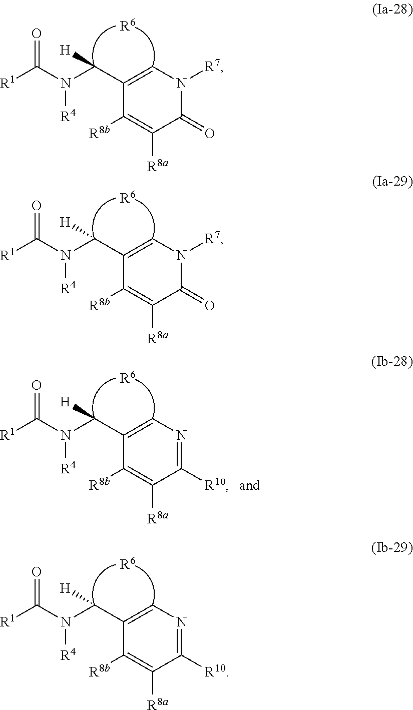

17. The compound of claim 1, wherein ring B 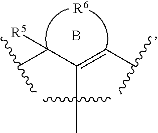
which is formed by R⁶ and the carbon atoms to which R⁶ is attached, is selected from the group consisting of:

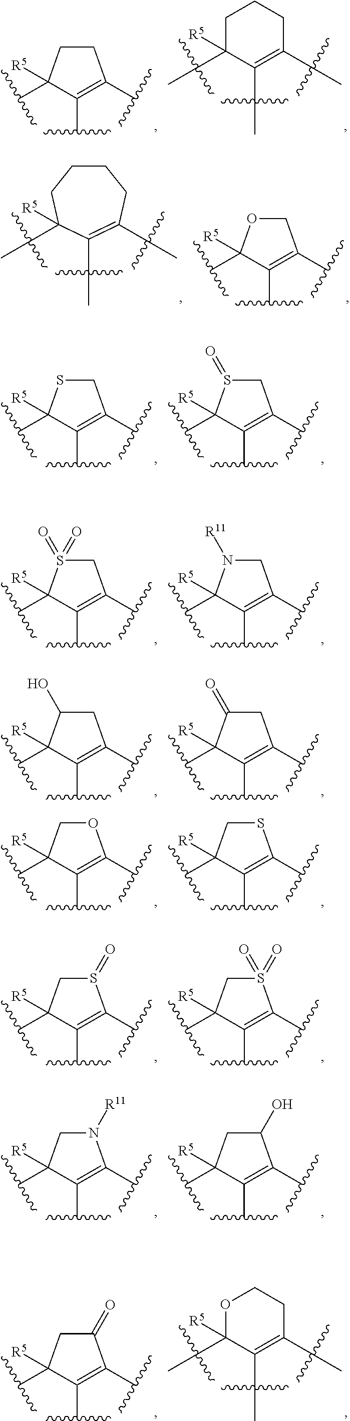

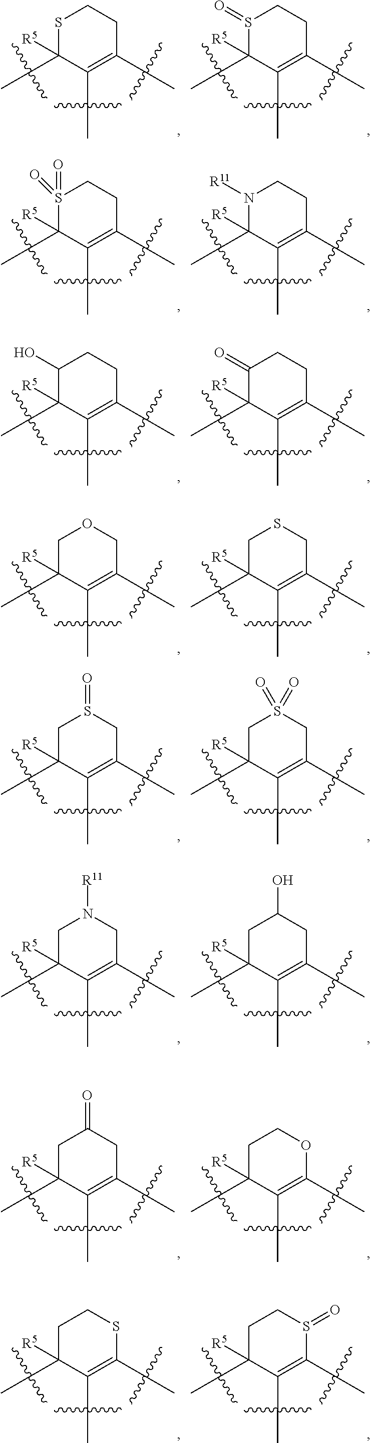

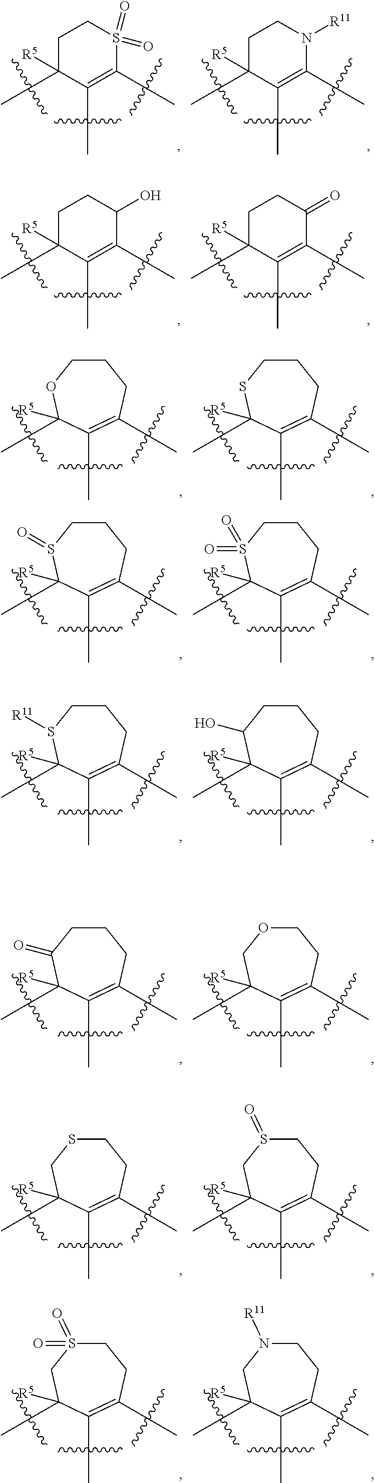

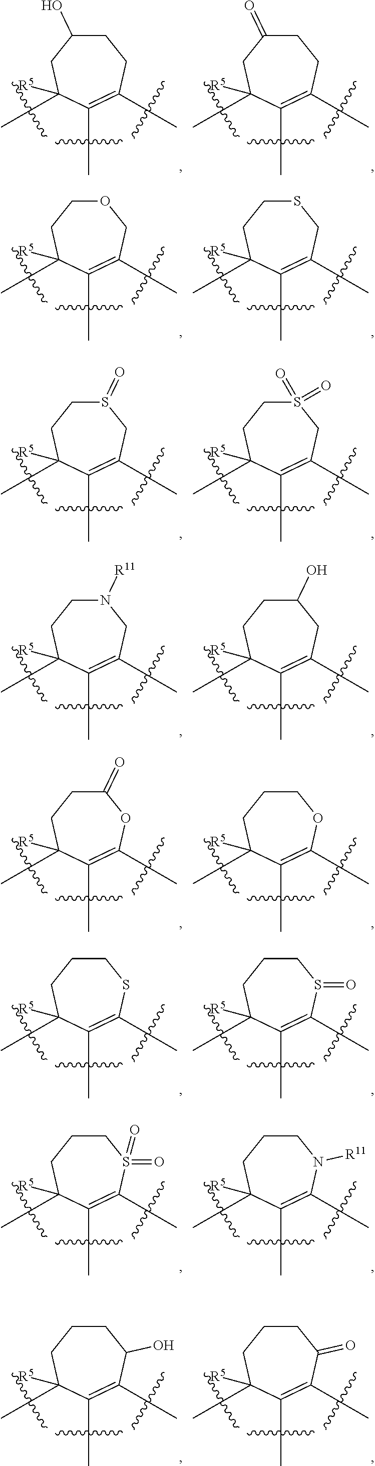

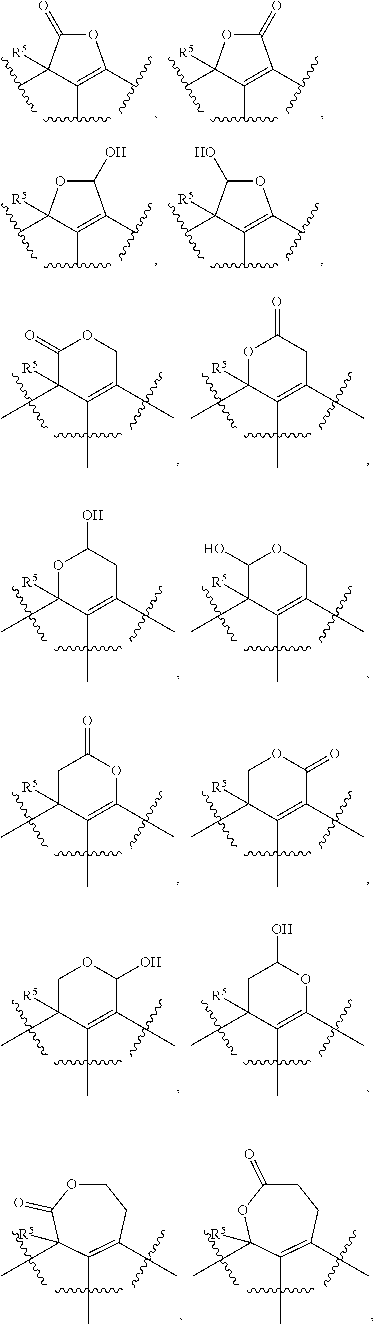

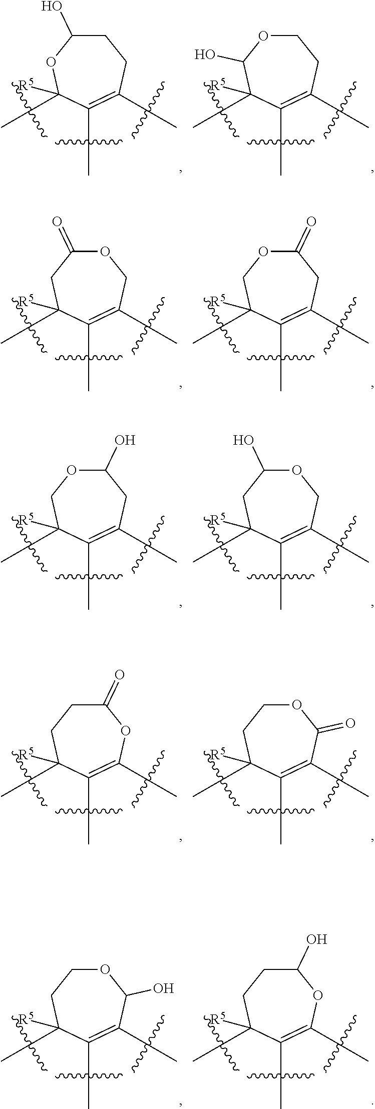

18. The compound of claim 1, which is at least one selected from the group consisting of:
3-(3-chloro-4-fluorophenyl)-1-isobutyl-1-(2-oxo-4-(trifluoromethyl)-1,2,5,6,7,8-hexahydroquinolin-5-yl)urea;
3-(3-chloro-4-fluorophenyl)-1-methyl-1-(2-oxo-4-(trifluoromethyl)-1,2,5,6,7,8-hexahydroquinolin-5-yl)urea;
3-(3-chloro-4-fluorophenyl)-1-(3-hydroxypropyl)-1-(2-oxo-4-(trifluoromethyl)-1,2,5,6,7,8-hexahydroquinolin-5-yl)urea;
3-(3-chloro-4-fluorophenyl)-1-isobutyl-1-(2-oxo-4-(trifluoromethyl)-2,5,6,7-tetrahydro-1H-cyclopenta[b]pyridin-5-yl)urea;
3-(3,4-difluorophenyl)-1-isobutyl-1-(2-oxo-4-(trifluoromethyl)-2,5,6,7-tetrahydro-1H-cyclopenta[b]pyridin-5-yl)urea;
3-(3-chloro-4-fluorophenyl)-1-methyl-1-(6-oxo-1,2,3,4,5,6-hexahydrophenanthridin-1-yl)urea;
3-(3-chloro-4-fluorophenyl)-1-(3-hydroxypropyl)-1-(6-oxo-1,2,3,4,5,6-hexahydrophenanthridin-1-yl)urea;
3-(3-chloro-4-fluorophenyl)-1-isobutyl-1-(6-oxo-1,2,3,4,5,6-hexahydrophenanthridin-1-yl)urea;
3-(3-chloro-4-fluorophenyl)-1-(8-fluoro-6-oxo-1,2,3,4,5,6-hexahydrophenanthridin-1-yl)-1-methylurea;
3-(3-chloro-4-fluorophenyl)-1-(8-fluoro-6-oxo-1,2,3,4,5,6-hexahydrophenanthridin-1-yl)-1-isobutylurea;
3-(3-chloro-4-fluorophenyl)-1-(8-fluoro-6-oxo-1,2,3,4,5,6-hexahydrophenanthridin-1-yl)-1-(3-hydroxypropyl)urea;
3-(3-chloro-4-fluorophenyl)-1-methyl-1-(5-oxo-2,3,4,5-tetrahydro-1H-cyclopenta[c]isoquinolin-1-yl)urea;
3-(3-chloro-4-fluorophenyl)-1-isobutyl-1-(5-oxo-2,3,4,5-tetrahydro-1H-cyclopenta[c]isoquinolin-1-yl)urea;
3-(3-chloro-4-fluorophenyl)-1-(3-hydroxypropyl)-1-(5-oxo-2,3,4,5-tetrahydro-1H-cyclopenta[c]isoquinolin-1-yl)urea;
3-(3-chloro-4-fluorophenyl)-1-(8,9-difluoro-6-oxo-1,2,3,4,5,6-hexahydrophenanthridin-1-yl)-1-methylurea;
3-(3-chloro-4-fluorophenyl)-1-methyl-1-(6-oxo-1,2,3,4,5,6-hexahydrophenanthridin-1-yl)urea;
3-(3-chloro-4-fluorophenyl)-1-methyl-1-(6-oxo-1,4,5,6-tetrahydro-2H-pyrano[3,4-c]isoquinolin-1-yl)urea;
1-(8,9-difluoro-6-oxo-1,2,3,4,5,6-hexahydrophenanthridin-1-yl)-3-(4-fluorophenyl)-1-methylurea;
3-(3-chloro-4-fluorophenyl)-1-(8,10-difluoro-6-oxo-1,2,3,4,5,6-hexahydrophenanthridin-1-yl)-1-methylurea;
3-(3-chloro-4-fluorophenyl)-1-methyl-1-(5-oxo-6,7,8,9,10,11-hexahydro-5H-cyclohepta[c]isoquinolin-11-yl)urea;
3-(3-chloro-4-fluorophenyl)-1-(3-hydroxypropyl)-1-(5-oxo-6,7,8,9,10,11-hexahydro-5H-cyclohepta[c]isoquinolin-11-yl)urea;
3-(3-chloro-4-fluorophenyl)-1-(8-fluoro-6-oxo-1,4,5,6-tetrahydro-2H-pyrano[3,4-c]isoquinolin-1-yl)-1-methylurea;
3-(3-chloro-4-fluorophenyl)-1-(8,9-difluoro-6-oxo-1,4,5,6-tetrahydro-2H-pyrano[3,4-c]isoquinolin-1-yl)-1-methylurea;
3-(3-chloro-4-fluorophenyl)-1-(8,10-difluoro-6-oxo-1,4,5,6-tetrahydro-2H-pyrano[3,4-c]isoquinolin-1-yl)-1-methylurea;
3-(3-chloro-4-fluorophenyl)-1-methyl-1-(3-methyl-6-oxo-1,2,3,4,5,6-hexahydrophenanthridin-1-yl)urea;
3-(3-chloro-4-fluorophenyl)-1-(3,3-dimethyl-6-oxo-1,2,3,4,5,6-hexahydrophenanthridin-1-yl)-1-methylurea;
3-(3-chloro-4-fluorophenyl)-1-(7,8-difluoro-6-oxo-1,2,3,4,5,6-hexahydrophenanthridin-1-yl)-1-methylurea;
3-(3-chloro-5-fluorophenyl)-1-methyl-1-(6-oxo-1,4,5,6-tetrahydro-2H-pyrano[3,4-c]isoquinolin-1-yl)urea;
3-(3-chloro-4-fluorophenyl)-1-isobutyl-1-(6-oxo-1,4,5,6-tetrahydro-2H-pyrano[3,4-c]isoquinolin-1-yl)urea;
3-(3-chloro-4-fluorophenyl)-1-methyl-1-(5-methyl-6-oxo-1,2,3,4,5,6-hexahydrophenanthridin-1-yl)urea;
3-(3,4-difluorophenyl)-1-(8-fluoro-6-oxo-1,2,3,4,5,6-hexahydrophenanthridin-1-yl)-1-methylurea;
1-(8-fluoro-6-oxo-1,2,3,4,5,6-hexahydrophenanthridin-1-yl)-1-methyl-3-(3,4,5-trifluorophenyl)urea;
3-(3-chloro-4-fluorophenyl)-1-(6-methoxy-1,2,3,4-tetrahydrophenanthridin-1-yl)-1-methylurea;
3-(3-chloro-4-fluorophenyl)-1-(7,8-difluoro-6-oxo-1,4,5,6-tetrahydro-2H-pyrano[3,4-c]isoquinolin-1-yl)-1-methylurea;
3-(3-chloro-4-fluorophenyl)-1-(8-fluoro-6-oxo-1,4,5,6-tetrahydro-2H-pyrano[3,4-c]isoquinolin-1-yl)-1-isobutylurea;
3-(3-chloro-4-fluorophenyl)-1-ethyl-1-(8-fluoro-6-oxo-1,4,5,6-tetrahydro-2H-pyrano[3,4-c]isoquinolin-1-yl)urea;
3-(3-chloro-4-fluorophenyl)-1-(8,9-difluoro-6-oxo-1,4,5,6-tetrahydro-2H-pyrano[3,4-c]isoquinolin-1-yl)-1-ethylurea;
3-(3-chloro-4-fluorophenyl)-1-methyl-1-(6-oxo-1,2,3,4,5,6,7,8,9,10-decahydrophenanthridin-1-yl)urea;
3-(3,4-difluorophenyl)-1-methyl-1-(6-oxo-1,2,3,4,5,6,7,8,9,10-decahydrophenanthridin-1-yl)urea;
3-(3,4-difluorophenyl)-1-ethyl-1-(6-oxo-1,2,3,4,5,6,7,8,9,10-decahydrophenanthridin-1-yl)urea;
3-(3-chloro-4-fluorophenyl)-1-ethyl-1-(6-oxo-1,2,3,4,5,6,7,8,9,10-decahydrophenanthridin-1-yl)urea;
3-(3-chloro-4-fluorophenyl)-1-(8-chloro-6-oxo-1,4,5,6-tetrahydro-2H-pyrano[3,4-c]isoquinolin-1-yl)-1-methylurea;
3-(3-chloro-4-fluorophenyl)-1-(8-chloro-6-oxo-1,4,5,6-tetrahydro-2H-pyrano[3,4-c]isoquinolin-1-yl)-1-ethylurea;
3-(4-fluoro-3-methylphenyl)-1-(8-fluoro-6-oxo-1,4,5,6-tetrahydro-2H-pyrano[3,4-c]isoquinolin-1-yl)-1-methylurea;
1-(8-chloro-6-oxo-1,4,5,6-tetrahydro-2H-pyrano[3,4-c]isoquinolin-1-yl)-3-(4-fluoro-3-methylphenyl)-1-methylurea;
1-(8-chloro-6-oxo-1,4,5,6-tetrahydro-2H-pyrano[3,4-c]isoquinolin-1-yl)-1-ethyl-3-(4-fluoro-3-methylphenyl)urea;
3-(3-chloro-4-fluorophenyl)-1-methyl-1-(5-oxo-1,4,5,6,7,8,9,10-octahydro-2H-pyrano[3,4-c]quinolin-10-yl)urea;
3-(3,4-difluorophenyl)-1-methyl-1-(5-oxo-1,4,5,6,7,8,9,10-octahydro-2H-pyrano[3,4-c]quinolin-10-yl)urea;
3-(3-cyano-4-fluorophenyl)-1-(8-fluoro-6-oxo-1,4,5,6-tetrahydro-2H-pyrano[3,4-c]isoquinolin-1-yl)-1-methylurea;
1-(8-chloro-6-oxo-1,4,5,6-tetrahydro-2H-pyrano[3,4-c]isoquinolin-1-yl)-3-(3-cyano-4-fluorophenyl)-1-methylurea;
1-(8-chloro-6-oxo-1,4,5,6-tetrahydro-2H-pyrano[3,4-c]isoquinolin-1-yl)-3-(3-cyano-4-fluorophenyl)-1-ethylurea;
1-(3-chloro-4-fluorophenyl)-3-(8-fluoro-6-oxo-1,4,5,6-tetrahydro-2H-pyrano[3,4-c]isoquinolin-1-yl)urea;
1-(3-chloro-4-fluorophenyl)-3-(8,9-difluoro-6-oxo-1,4,5,6-tetrahydro-2H-pyrano[3,4-c]isoquinolin-1-yl)urea;
1-(8,9-difluoro-6-oxo-1,4,5,6-tetrahydro-2H-pyrano[3,4-c]isoquinolin-1-yl)-3-(4-fluoro-3-methylphenyl)-1-methylurea;
3-(3-chloro-4-fluorophenyl)-1-(9-fluoro-6-oxo-1,4,5,6-tetrahydro-2H-pyrano[3,4-c]isoquinolin-1-yl)-1-methylurea;
3-(3-chloro-4-fluorophenyl)-1-ethyl-1-(9-fluoro-6-oxo-1,4,5,6-tetrahydro-2H-pyrano[3,4-c]isoquinolin-1-yl)urea;
3-(4-fluoro-3-methylphenyl)-1-(9-fluoro-6-oxo-1,4,5,6-tetrahydro-2H-pyrano[3,4-c]isoquinolin-1-yl)-1-methylurea;
1-ethyl-3-(4-fluoro-3-methylphenyl)-1-(9-fluoro-6-oxo-1,4,5,6-tetrahydro-2H-pyrano[3,4-c]isoquinolin-1-yl)urea;
3-(3-cyano-4-fluorophenyl)-1-(9-fluoro-6-oxo-1,4,5,6-tetrahydro-2H-pyrano[3,4-c]isoquinolin-1-yl)-1-methylurea;
3-(3-cyano-4-fluorophenyl)-1-ethyl-1-(9-fluoro-6-oxo-1,4,5,6-tetrahydro-2H-pyrano[3,4-c]isoquinolin-1-yl)urea;
3-(3-chloro-4-fluorophenyl)-1-methyl-1-(5-oxo-3,4,5,6,7,8,9,10-octahydro-1H-pyrano[4,3-c]quinolin-10-yl)urea;
3-(3,4-difluorophenyl)-1-methyl-1-(5-oxo-3,4,5,6,7,8,9,10-octahydro-1H-pyrano[4,3-c]quinolin-10-yl)urea;
3-(3-chloro-4-fluorophenyl)-1-methyl-1-(5-oxo-4,5,6,7,9,10-hexahydro-1H,3H-dipyrano[3,4-b:3′,4′-d]pyridin-10-yl)urea;
3-(3,4-difluorophenyl)-1-methyl-1-(5-oxo-4,5,6,7,9,10-hexahydro-1H,3H-dipyrano[3,4-b:3′,4′-d]pyridin-10-yl)urea;
3-(3-chloro-4-fluorophenyl)-1-(8-cyano-6-oxo-1,4,5,6-tetrahydro-2H-pyrano[3,4-c]isoquinolin-1-yl)-1-methylurea;
3-(3-chloro-4-fluorophenyl)-1-(8-cyano-6-oxo-1,4,5,6-tetrahydro-2H-pyrano[3,4-c]isoquinolin-1-yl)-1-ethylurea;
3-(3-chloro-4-fluorophenyl)-1-methyl-1-(6-oxo-1,2,4,5,6,7,9,10-octahydrodipyrano[3,4-b:4′,3′-d]pyridin-1-yl)urea;
3-(3,4-difluorophenyl)-1-methyl-1-(6-oxo-1,2,4,5,6,7,9,10-octahydrodipyrano[3,4-b:4′,3′-d]pyridin-1-yl)urea;
3-(3-chloro-4-fluorophenyl)-1-methyl-1-(6-oxo-1,4,5,6-tetrahydro-2H-pyrano[3,4-b]thieno[3,2-d]pyridin-1-yl)urea;
3-(3-chloro-4-fluorophenyl)-1-methyl-1-(4-oxo-4,6,8,9-tetrahydro-5H-pyrano[3,4-b]thieno[2,3-d]pyridin-9-yl)urea;
3-(3,5-dichloro-4-fluorophenyl)-1-(8-fluoro-6-oxo-1,4,5,6-tetrahydro-2H-pyrano[3,4-c]isoquinolin-1-yl)-1-methylurea;
3-(3,4-difluorophenyl)-1-(8-fluoro-6-oxo-1,4,5,6-tetrahydro-2H-pyrano[3,4-c]isoquinolin-1-yl)-1-isobutylurea;
1-(8,9-difluoro-6-oxo-1,4,5,6-tetrahydro-2H-pyrano[3,4-c]isoquinolin-1-yl)-1-methyl-3-phenylurea;
1-(8,9-difluoro-6-oxo-1,4,5,6-tetrahydro-2H-pyrano[3,4-c]isoquinolin-1-yl)-3-(4-fluorophenyl)-1-methylurea;
3-(3-chloro-4-fluorophenyl)-1-methyl-1-(4-oxo-4,5,8,9-tetrahydro-6H-pyrano[3,4-b]thieno[3,4-d]pyridin-9-yl)urea;
3-(3-cyano-4-fluorophenyl)-1-(8,9-difluoro-6-oxo-1,4,5,6-tetrahydro-2H-pyrano[3,4-c]isoquinolin-1-yl)-1-methylurea;
3-(3-chloro-4,5-difluorophenyl)-1-(8-fluoro-6-oxo-1,4,5,6-tetrahydro-2H-pyrano[3,4-c]isoquinolin-1-yl)-1-methylurea;
3-(3-chloro-4-fluorophenyl)-1-(8-fluoro-6-oxo-1,4,5,6-tetrahydro-2H-pyrano[3,4-c]isoquinolin-1-yl)-1-(3-hydroxypropyl)urea;
3-(3-chloro-4-fluorophenyl)-1-(8-fluoro-6-oxo-1,4,5,6-tetrahydro-2H-pyrano[3,4-c]isoquinolin-1-yl)-1-(2-hydroxy-2-methylpropyl)urea;
1-(8-fluoro-6-oxo-1,4,5,6-tetrahydro-2H-pyrano[3,4-c]isoquinolin-1-yl)-1-isobutyl-3-(3,4,5-trifluorophenyl)urea;
3-(3-cyano-4-fluorophenyl)-1-(8-fluoro-6-oxo-1,4,5,6-tetrahydro-2H-pyrano[3,4-c]isoquinolin-1-yl)-1-isobutylurea;
1-(8,9-difluoro-6-oxo-1,4,5,6-tetrahydro-2H-pyrano[3,4-c]isoquinolin-1-yl)-3-(3,4-difluorophenyl)-1-methylurea;
3-(3-chlorophenyl)-1-(8,9-difluoro-6-oxo-1,4,5,6-tetrahydro-2H-pyrano[3,4-c]isoquinolin-1-yl)-1-methylurea;
3-(3-cyano-4-fluorophenyl)-1-(8,9-difluoro-6-oxo-1,4,5,6-tetrahydro-2H-pyrano[3,4-c]isoquinolin-1-yl)-1-isobutylurea;
3-(3-(difluoromethyl)-4-fluorophenyl)-1-(8-fluoro-6-oxo-1,4,5,6-tetrahydro-2H-pyrano[3,4-c]isoquinolin-1-yl)-1-methylurea;
1-(8,9-difluoro-6-oxo-1,4,5,6-tetrahydro-2H-pyrano[3,4-c]isoquinolin-1-yl)-3-(3-(difluoromethyl)-4-fluorophenyl)-1-methylurea;
3-(4-fluoro-3-methylphenyl)-1-(8-fluoro-6-oxo-1,4,5,6-tetrahydro-2H-pyrano[3,4-c]isoquinolin-1-yl)-1-isobutylurea;
1-(8,9-difluoro-6-oxo-1,4,5,6-tetrahydro-2H-pyrano[3,4-c]isoquinolin-1-yl)-1-methyl-3-(3,4,5-trifluorophenyl)urea;
1-(3-cyano-4-fluorophenyl)-3-(8,9-difluoro-6-oxo-1,4,5,6-tetrahydro-2H-pyrano[3,4-c]isoquinolin-1-yl)urea;
2-(3-(3-chloro-4-fluorophenyl)-1-(8,9-difluoro-6-oxo-1,4,5,6-tetrahydro-2H-pyrano[3,4-c]isoquinolin-1-yl)ureido)ethane-1-sulfonamide;
3-(3-cyano-4-fluorophenyl)-1-(8,9-difluoro-6-oxo-1,4,5,6-tetrahydro-2H-pyrano[3,4-c]isoquinolin-1-yl)-1-ethylurea;
3-(3-chloro-4-fluorophenyl)-1-(8,9-difluoro-6-oxo-1,4,5,6-tetrahydro-2H-pyrano[3,4-c]isoquinolin-1-yl)-1-(2-(methylsulfonyl)ethyl)urea;
3-(4-chloro-3-fluorophenyl)-1-(8,9-difluoro-6-oxo-1,4,5,6-tetrahydro-2H-pyrano[3,4-c]isoquinolin-1-yl)-1-methylurea;
3-(4-chloro-3-cyanophenyl)-1-(8,9-difluoro-6-oxo-1,4,5,6-tetrahydro-2H-pyrano[3,4-c]isoquinolin-1-yl)-1-methylurea;
3-(3,4-dichlorophenyl)-1-(8,9-difluoro-6-oxo-1,4,5,6-tetrahydro-2H-pyrano[3,4-c]isoquinolin-1-yl)-1-methylurea;
1-(8,9-difluoro-6-oxo-1,2,3,4,5,6-hexahydrophenanthridin-1-yl)-1-methyl-3-(1-(trifluoromethyl)cyclopropyl)urea;
1-(8,9-difluoro-6-oxo-1,4,5,6-tetrahydro-2H-pyrano[3,4-c]isoquinolin-1-yl)-1-methyl-3-(1-(trifluoromethyl)cyclopropyl)urea;
3-(3-Chloro-4-fluorophenyl)-1-(8,9-difluoro-6-oxo-1,4,5,6-tetrahydro-2H-pyrano[3,4-c]isoquinolin-1-yl-1-d)-1-(methyl-d3)urea;
3-(3-Chloro-4-methoxyphenyl)-1-(8,9-difluoro-6-oxo-1,4,5,6-tetrahydro-2H-pyrano[3,4-c]isoquinolin-1-yl)-1-methylurea;
3-(3-Chloro-4-hydroxyphenyl)-1-(8,9-difluoro-6-oxo-1,4,5,6-tetrahydro-2H-pyrano[3,4-c]isoquinolin-1-yl)-1-methylurea;
3-(3-Chloro-4-fluorophenyl)-1-(8,9-difluoro-6-oxo-1,2,3,4,5,6-hexahydrobenzo[c][1,7]naphthyridin-1-yl)-1-methylurea;
3-(3-Chloro-4-fluorophenyl)-1-(8,9-difluoro-4-hydroxy-6-oxo-1,4,5,6-tetrahydro-2H-pyrano[3,4-c]isoquinolin-1-yl)-1-methylurea;
3-(3-chloro-4-Fluorophenyl)-1-(8,9-difluoro-4,6-dioxo-1,4,5,6-tetrahydro-2H-pyrano[3,4-c]isoquinolin-1-yl)-1-methylurea;
3-(3-Chloro-4-fluorophenyl)-1-(8-fluoro-6-oxo-1,2,3,4,5,6-hexahydrobenzo[c][1,7]naphthyridin-1-yl)-1-methylurea;
3-(3-Chloro-4-fluorophenyl)-1-(8-fluoro-3-methyl-6-oxo-1,2,3,4,5,6-hexahydrobenzo[c][1,7]naphthyridin-1-yl)-1-methylurea;
1-(3-Acetyl-8-fluoro-6-oxo-1,2,3,4,5,6-hexahydrobenzo[c][1,7]naphthyridin-1-yl)-3-(3-chloro-4-fluorophenyl)-1-methylurea;
1-(3-Acetyl-8,9-difluoro-6-oxo-1,2,3,4,5,6-hexahydrobenzo[c][1,7]naphthyridin-1-yl)-3-(3-chloro-4-fluorophenyl)-1-methylurea;
3-(3-Cyano-4-fluorophenyl)-1-(8-fluoro-6-oxo-1,2,3,4,5,6-hexahydrobenzo[c][1,7]naphthyridin-1-yl)-1-methylurea;
3-(3-Cyano-4-fluorophenyl)-1-(8-fluoro-3-methyl-6-oxo-1,2,3,4,5,6-hexahydrobenzo[c][1,7]naphthyridin-1-yl)-1-methylurea;
1-(3-Acetyl-8-fluoro-6-oxo-1,2,3,4,5,6-hexahydrobenzo[c][1,7]naphthyridin-1-yl)-3-(3-cyano-4-fluorophenyl)-1-methylurea;
3-(3-Chloro-4-fluorophenyl)-1-(8,9-difluoro-3-methyl-6-oxo-1,2,3,4,5,6-hexahydrobenzo[c][1,7]naphthyridin-1-yl)-1-methylurea;
3-(3-Chloro-4-fluorophenyl)-1-(8-fluoro-3-(2-hydroxyethyl)-6-oxo-1,2,3,4,5,6-hexahydrobenzo[c][1,7]naphthyridin-1-yl)-1-methylurea;
3-(3-Cyano-4-fluorophenyl)-1-(8-fluoro-3-(2-hydroxyethyl)-6-oxo-1,2,3,4,5,6-hexahydrobenzo[c][1,7]naphthyridin-1-yl)-1-methylurea;
3-(3-Cyano-4-fluorophenyl)-1-(8,9-difluoro-3-methyl-6-oxo-1,2,3,4,5,6-hexahydrobenzo[c][1,7]naphthyridin-1-yl)-1-methylurea;
3-(3-Cyano-4-fluorophenyl)-1-(8,9-difluoro-6-oxo-1,2,3,4,5,6-hexahydrobenzo[c][1,7]naphthyridin-1-yl)-1-methylurea;
1-(3-Acetyl-8,9-difluoro-6-oxo-1,2,3,4,5,6-hexahydrobenzo[c][1,7]naphthyridin-1-yl)-3-(3-cyano-4-fluorophenyl)-1-methylurea;
3-(3-Chloro-4-fluorophenyl)-1-(8,9-difluoro-3-(2-hydroxyethyl)-6-oxo-1,2,3,4,5,6-hexahydrobenzo[c][1,7]naphthyridin-1-yl)-1-methylurea;
3-(3-Cyano-4-fluorophenyl)-1-(8,9-difluoro-3-(2-hydroxyethyl)-6-oxo-1,2,3,4,5,6-hexahydrobenzo[c][1,7]naphthyridine-1-yl)-1-methylurea;
3-(3-Chloro-4-fluorophenyl)-1-(8-fluoro-6-oxo-1,4,5,6-tetrahydro-2H-thiopyrano[3,4-c]isoquinolin-1-yl)-1-methylurea;
3-(3-Cyano-4-fluorophenyl)-1-(8-fluoro-6-oxo-1,4,5,6-tetrahydro-2H-thiopyrano[3,4-c]isoquinolin-1-yl)-1-methylurea;
3-(3-Chloro-4-fluorophenyl)-1-(8,9-difluoro-6-oxo-1,4,5,6-tetrahydro-2H-thiopyrano[3,4-c]isoquinolin-1-yl)-1-methylurea;
3-(3-Cyano-4-fluorophenyl)-1-(8,9-difluoro-6-oxo-1,4,5,6-tetrahydro-2H-thiopyrano[3,4-c]isoquinolin-1-yl)-1-methylurea;
1-(8,9-Difluoro-6-oxo-1,2,3,4,5,6-hexahydrobenzo[c][1,7]naphthyridin-1-yl)-3-(3,4-difluorophenyl)-1-methylurea;
3-(3-Chloro-4-fluorophenyl)-1-(8-fluoro-3-oxido-6-oxo-1,4,5,6-tetrahydro-2H-thiopyrano[3,4-c]isoquinolin-1-yl)-1-methylurea;
3-(3-Chloro-4-fluorophenyl)-1-(8-fluoro-3,3-dioxido-6-oxo-1,4,5,6-tetrahydro-2H-thiopyrano[3,4-c]isoquinolin-1-yl)-1-methylurea;
3-(3-Cyano-4-fluorophenyl)-1-(8-fluoro-3-oxido-6-oxo-1,4,5,6-tetrahydro-2H-thiopyrano[3,4-c]isoquinolin-1-yl)-1-methylurea;
3-(3-Chloro-4-fluorophenyl)-1-(8,9-difluoro-3,3-dioxido-6-oxo-1,4,5,6-tetrahydro-2H-thiopyrano[3,4-c]isoquinolin-1-yl)-1-methylurea;
3-(3-Cyano-4-fluorophenyl)-1-(8,9-difluoro-3,3-dioxido-6-oxo-1,4,5,6-tetrahydro-2H-thiopyrano[3,4-c]isoquinolin-1-yl)-1-methylurea;
3-(3-Chloro-4-fluorophenyl)-1-(8,9-difluoro-3-oxido-6-oxo-1,4,5,6-tetrahydro-2H-thiopyrano[3,4-c]isoquinolin-1-yl)-1-methylurea;
3-(3-Cyano-4-fluorophenyl)-1-(8,9-difluoro-3-oxido-6-oxo-1,4,5,6-tetrahydro-2H-thiopyrano[3,4-c]isoquinolin-1-yl)-1-methylurea;
3-(3-Cyano-4-fluorophenyl)-1-(8-fluoro-3,3-dioxido-6-oxo-1,4,5,6-tetrahydro-2H-thiopyrano[3,4-c]isoquinolin-1-yl)-1-methylurea;
1-(8,9-difluoro-6-oxo-1,2,3,4,5,6-hexahydrobenzo[c][1,7]naphthyridin-1-yl)-3-(3-(difluoromethyl)-4-fluorophenyl)-1-methylurea;
N-(8,9-difluoro-6-oxo-1,4,5,6-tetrahydro-2H-pyrano[3,4-c]isoquinolin-1-yl)-N-methylisoindoline-2-carboxamide;
5-chloro-N-(8,9-difluoro-6-oxo-1,4,5,6-tetrahydro-2H-pyrano[3,4-c]isoquinolin-1-yl)-N-methylisoindoline-2-carboxamide;
5-bromo-N-(8,9-difluoro-6-oxo-1,4,5,6-tetrahydro-2H-pyrano[3,4-c]isoquinolin-1-yl)-N-methylisoindoline-2-carboxamide;
5-fluoro-N-(8,9-difluoro-6-oxo-1,4,5,6-tetrahydro-2H-pyrano[3,4-c]isoquinolin-1-yl)-N-methylisoindoline-2-carboxamide;
N-(8,9-difluoro-6-oxo-1,2,3,4,5,6-hexahydrobenzo[c][1,7]naphthyridin-1-yl)-N-methylisoindoline-2-carboxamide;
N-(8,9-difluoro-6-oxo-1,2,3,4,5,6-hexahydrobenzo[c][1,7]naphthyridin-1-yl)-5-fluoro-N-methylisoindoline-2-carboxamide;
N-(8,9-difluoro-6-oxo-1,2,3,4,5,6-hexahydrobenzo[c][1,7]naphthyridin-1-yl)-5-chloro-N-methylisoindoline-2-carboxamide;
N-(8,9-difluoro-6-oxo-1,2,3,4,5,6-hexahydrobenzo[c][1,7]naphthyridin-1-yl)-5-bromo-N-methylisoindoline-2-carboxamide;
N-(8,9-difluoro-6-oxo-1,4,5,6-tetrahydro-2H-pyrano[3,4-c]isoquinolin-1-yl)-N-methyl-5-(trifluoromethyl)isoindoline-2-carboxamide;
N-(8,9-difluoro-6-oxo-1,2,3,4,5,6-hexahydrobenzo[c][1,7]naphthyridin-1-yl)-N-methyl-5-(trifluoromethyl)isoindoline-2-carboxamide;
1-(8,9-difluoro-5-methyl-6-oxo-1,4,5,6-tetrahydro-2H-pyrano[3,4-c]isoquinolin-1-yl)-3-(3-(difluoromethyl)-4-fluorophenyl)-1-methylurea;
1-(8,9-difluoro-6-methoxy-1,4-dihydro-2H-pyrano[3,4-c]isoquinolin-1-yl)-3-(3-(difluoromethyl)-4-fluorophenyl)-1-methylurea;
1-(8,9-difluoro-6-(methylamino)-1,4-dihydro-2H-pyrano[3,4-c]isoquinolin-1-yl)-3-(3-(difluoromethyl)-4-fluorophenyl)-1-methylurea
1-(8,9-difluoro-6-((2-hydroxyethyl)amino)-1,4-dihydro-2H-pyrano[3,4-c]isoquinolin-1-yl)-3-(3-(difluoromethyl)-4-fluorophenyl)-1-methylurea;
1-(8,9-difluoro-6-((2-aminoethyl)amino)-1,4-dihydro-2H-pyrano[3,4-c]isoquinolin-1-yl)-3-(3-(difluoromethyl)-4-fluorophenyl)-1-methylurea
3-(3-chloro-4-fluorophenyl)-1-(8,9-difluoro-6-((2-aminoethyl)amino)-1,4-dihydro-2H-pyrano[3,4-c]isoquinolin-1-yl)-1-methylurea;
3-(3-chloro-4-fluorophenyl)-1-(8,9-difluoro-6-(methylamino)-1,4-dihydro-2H-pyrano[3,4-c]isoquinolin-1-yl)-1-methylurea;
1-(8,9-difluoro-5-methyl-6-oxo-1,4,5,6-tetrahydro-2H-pyrano[3,4-c]isoquinolin-1-yl)-3-(3-chloro-4-fluorophenyl)-1-methylurea;
3-(3-chloro-4-fluorophenyl)-1-(8,9-difluoro-6-methoxy-1,4-dihydro-2H-pyrano[3,4-c]isoquinolin-1-yl)-1-methylurea;
3-(3-chloro-4-fluorophenyl)-1-(8,9-difluoro-6-((2-hydroxyethyl)amino)-1,4-dihydro-2H-pyrano[3,4-c]isoquinolin-1-yl)-1-methylurea;
1-(8,9-difluoro-5-(2-hydroxyethyl)-6-oxo-1,4,5,6-tetrahydro-2H-pyrano[3,4-c]isoquinolin-1-yl)-3-(3-(difluoromethyl)-4-fluorophenyl)-1-methylurea;
1-(6-(2-aminoethoxy)-8,9-difluoro-1,4-dihydro-2H-pyrano[3,4-c]isoquinolin-1-yl)-3-(3-(difluoromethyl)-4-fluorophenyl)-1-methylurea;
1-(5-(2-aminoethyl)-8,9-difluoro-6-oxo-1,4,5,6-tetrahydro-2H-pyrano[3,4-c]isoquinolin-1-yl)-3-(3-(difluoromethyl)-4-fluorophenyl)-1-methylurea;
3-(3-chloro-4-fluorophenyl)-1-(8,9-difluoro-5-(2-hydroxyethyl)-6-oxo-1,4,5,6-tetrahydro-2H-pyrano[3,4-c]isoquinolin-1-yl)-1-methylurea;
3-(3-chloro-4-fluorophenyl)-1-(8,9-difluoro-6-(2-hydroxyethoxy)-1,4-dihydro-2H-pyrano[3,4-c]isoquinolin-1-yl)-1-methylurea;
1-(8,9-difluoro-6-(2-hydroxyethoxy)-1,4-dihydro-2H-pyrano[3,4-c]isoquinolin-1-yl)-3-(3-(difluoromethyl)-4-fluorophenyl)-1-methylurea;
1-(5-(2-aminoethyl)-8,9-difluoro-6-oxo-1,4,5,6-tetrahydro-2H-pyrano[3,4-c]isoquinolin-1-yl)-3-(3-chloro-4-fluorophenyl)-1-methylurea;
1-(6-(2-aminoethoxy)-8,9-difluoro-1,4-dihydro-2H-pyrano[3,4-c]isoquinolin-1-yl)-3-(3-chloro-4-fluorophenyl)-1-methylurea; or a salt, solvate, prodrug, isotopically labelled derivative, stereoisomer, or tautomer thereof, or any mixtures thereof.

19. The compound of claim 1, which is at least one selected from the group consisting of:
(R)-3-(3-chloro-4-fluorophenyl)-1-isobutyl-1-(2-oxo-4-(trifluoromethyl)-1,2,5,6,7,8-hexahydroquinolin-5-yl)urea;
(S)-3-(3-chloro-4-fluorophenyl)-1-isobutyl-1-(2-oxo-4-(trifluoromethyl)-1,2,5,6,7,8-hexahydroquinolin-5-yl)urea;
(R)-3-(3-chloro-4-fluorophenyl)-1-methyl-1-(2-oxo-4-(trifluoromethyl)-1,2,5,6,7,8-hexahydroquinolin-5-yl)urea;
(S)-3-(3-chloro-4-fluorophenyl)-1-methyl-1-(2-oxo-4-(trifluoromethyl)-1,2,5,6,7,8-hexahydroquinolin-5-yl)urea;
(R)-3-(3-chloro-4-fluorophenyl)-1-(3-hydroxypropyl)-1-(2-oxo-4-(trifluoromethyl)-1,2,5,6,7,8-hexahydroquinolin-5-yl)urea;
(S)-3-(3-chloro-4-fluorophenyl)-1-(3-hydroxypropyl)-1-(2-oxo-4-(trifluoromethyl)-1,2,5,6,7,8-hexahydroquinolin-5-yl)urea;
(R)-3-(3-chloro-4-fluorophenyl)-1-isobutyl-1-(2-oxo-4-(trifluoromethyl)-2,5,6,7-tetrahydro-1H-cyclopenta[b]pyridin-5-yl)urea;
(S)-3-(3-chloro-4-fluorophenyl)-1-isobutyl-1-(2-oxo-4-(trifluoromethyl)-2,5,6,7-tetrahydro-1H-cyclopenta[b]pyridin-5-yl)urea;
(R)-3-(3,4-difluorophenyl)-1-isobutyl-1-(2-oxo-4-(trifluoromethyl)-2,5,6,7-tetrahydro-1H-cyclopenta[b]pyridin-5-yl)urea;
(S)-3-(3,4-difluorophenyl)-1-isobutyl-1-(2-oxo-4-(trifluoromethyl)-2,5,6,7-tetrahydro-1H-cyclopenta[b]pyridin-5-yl)urea;
(R)-3-(3-chloro-4-fluorophenyl)-1-methyl-1-(6-oxo-1,2,3,4,5,6-hexahydrophenanthridin-1-yl)urea;
(S)-3-(3-chloro-4-fluorophenyl)-1-methyl-1-(6-oxo-1,2,3,4,5,6-hexahydrophenanthridin-1-yl)urea;
(R)-3-(3-chloro-4-fluorophenyl)-1-(3-hydroxypropyl)-1-(6-oxo-1,2,3,4,5,6-hexahydrophenanthridin-1-yl)urea;
(S)-3-(3-chloro-4-fluorophenyl)-1-(3-hydroxypropyl)-1-(6-oxo-1,2,3,4,5,6-hexahydrophenanthridin-1-yl)urea;
(R)-3-(3-chloro-4-fluorophenyl)-1-isobutyl-1-(6-oxo-1,2,3,4,5,6-hexahydrophenanthridin-1-yl)urea;
(S)-3-(3-chloro-4-fluorophenyl)-1-isobutyl-1-(6-oxo-1,2,3,4,5,6-hexahydrophenanthridin-1-yl)urea;
(R)-3-(3-chloro-4-fluorophenyl)-1-(8-fluoro-6-oxo-1,2,3,4,5,6-hexahydrophenanthridin-1-yl)-1-methylurea;
(S)-3-(3-chloro-4-fluorophenyl)-1-(8-fluoro-6-oxo-1,2,3,4,5,6-hexahydrophenanthridin-1-yl)-1-methylurea;
(R)-3-(3-chloro-4-fluorophenyl)-1-(8-fluoro-6-oxo-1,2,3,4,5,6-hexahydrophenanthridin-1-yl)-1-isobutylurea;
(S)-3-(3-chloro-4-fluorophenyl)-1-(8-fluoro-6-oxo-1,2,3,4,5,6-hexahydrophenanthridin-1-yl)-1-isobutylurea;
(R)-3-(3-chloro-4-fluorophenyl)-1-(8-fluoro-6-oxo-1,2,3,4,5,6-hexahydrophenanthridin-1-yl)-1-(3-hydroxypropyl)urea;
(S)-3-(3-chloro-4-fluorophenyl)-1-(8-fluoro-6-oxo-1,2,3,4,5,6-hexahydrophenanthridin-1-yl)-1-(3-hydroxypropyl)urea;
(R)-3-(3-chloro-4-fluorophenyl)-1-methyl-1-(5-oxo-2,3,4,5-tetrahydro-1H-cyclopenta[c]isoquinolin-1-yl)urea;
(S)-3-(3-chloro-4-fluorophenyl)-1-methyl-1-(5-oxo-2,3,4,5-tetrahydro-1H-cyclopenta[c]isoquinolin-1-yl)urea;
(R)-3-(3-chloro-4-fluorophenyl)-1-isobutyl-1-(5-oxo-2,3,4,5-tetrahydro-1H-cyclopenta[c]isoquinolin-1-yl)urea;
(S)-3-(3-chloro-4-fluorophenyl)-1-isobutyl-1-(5-oxo-2,3,4,5-tetrahydro-1H-cyclopenta[c]isoquinolin-1-yl)urea;
(R)-3-(3-chloro-4-fluorophenyl)-1-(3-hydroxypropyl)-1-(5-oxo-2,3,4,5-tetrahydro-1H-cyclopenta[c]isoquinolin-1-yl)urea;
(S)-3-(3-chloro-4-fluorophenyl)-1-(3-hydroxypropyl)-1-(5-oxo-2,3,4,5-tetrahydro-1H-cyclopenta[c]isoquinolin-1-yl)urea;
(R)-3-(3-chloro-4-fluorophenyl)-1-(8,9-difluoro-6-oxo-1,2,3,4,5,6-hexahydrophenanthridin-1-yl)-1-methylurea;
(S)-3-(3-chloro-4-fluorophenyl)-1-(8,9-difluoro-6-oxo-1,2,3,4,5,6-hexahydrophenanthridin-1-yl)-1-methylurea;
(R)-3-(3-chloro-4-fluorophenyl)-1-methyl-1-(6-oxo-1,2,3,4,5,6-hexahydrophenanthridin-1-yl)urea;
(S)-3-(3-chloro-4-fluorophenyl)-1-methyl-1-(6-oxo-1,2,3,4,5,6-hexahydrophenanthridin-1-yl)urea;
(R)-3-(3-chloro-4-fluorophenyl)-1-methyl-1-(6-oxo-1,4,5,6-tetrahydro-2H-pyrano[3,4-c]isoquinolin-1-yl)urea;
(S)-3-(3-chloro-4-fluorophenyl)-1-methyl-1-(6-oxo-1,4,5,6-tetrahydro-2H-pyrano[3,4-c]isoquinolin-1-yl)urea;
(R)-1-(8,9-difluoro-6-oxo-1,2,3,4,5,6-hexahydrophenanthridin-1-yl)-3-(4-fluorophenyl)-1-methylurea;
(S)-1-(8,9-difluoro-6-oxo-1,2,3,4,5,6-hexahydrophenanthridin-1-yl)-3-(4-fluorophenyl)-1-methylurea;
(R)-3-(3-chloro-4-fluorophenyl)-1-(8,10-difluoro-6-oxo-1,2,3,4,5,6-hexahydrophenanthridin-1-yl)-1-methylurea;
(S)-3-(3-chloro-4-fluorophenyl)-1-(8,10-difluoro-6-oxo-1,2,3,4,5,6-hexahydrophenanthridin-1-yl)-1-methylurea;
(R)-3-(3-chloro-4-fluorophenyl)-1-methyl-1-(5-oxo-6,7,8,9,10,11-hexahydro-5H-cyclohepta[c]isoquinolin-11-yl)urea;
(S)-3-(3-chloro-4-fluorophenyl)-1-methyl-1-(5-oxo-6,7,8,9,10,11-hexahydro-5H-cyclohepta[c]isoquinolin-11-yl)urea;
(R)-3-(3-chloro-4-fluorophenyl)-1-(3-hydroxypropyl)-1-(5-oxo-6,7,8,9,10,11-hexahydro-5H-cyclohepta[c]isoquinolin-11-yl)urea;
(S)-3-(3-chloro-4-fluorophenyl)-1-(3-hydroxypropyl)-1-(5-oxo-6,7,8,9,10,11-hexahydro-5H-cyclohepta[c]isoquinolin-11-yl)urea;
(R)-3-(3-chloro-4-fluorophenyl)-1-(8-fluoro-6-oxo-1,4,5,6-tetrahydro-2H-pyrano[3,4-c]isoquinolin-1-yl)-1-methylurea;
(S)-3-(3-chloro-4-fluorophenyl)-1-(8-fluoro-6-oxo-1,4,5,6-tetrahydro-2H-pyrano[3,4-c]isoquinolin-1-yl)-1-methylurea;
(R)-3-(3-chloro-4-fluorophenyl)-1-(8,9-difluoro-6-oxo-1,4,5,6-tetrahydro-2H-pyrano[3,4-c]isoquinolin-1-yl)-1-methylurea;
(S)-3-(3-chloro-4-fluorophenyl)-1-(8,9-difluoro-6-oxo-1,4,5,6-tetrahydro-2H-pyrano[3,4-c]isoquinolin-1-yl)-1-methylurea;
(R)-3-(3-chloro-4-fluorophenyl)-1-(8,10-difluoro-6-oxo-1,4,5,6-tetrahydro-2H-pyrano[3,4-c]isoquinolin-1-yl)-1-methylurea;
(S)-3-(3-chloro-4-fluorophenyl)-1-(8,10-difluoro-6-oxo-1,4,5,6-tetrahydro-2H-pyrano[3,4-c]isoquinolin-1-yl)-1-methylurea;
3-(3-chloro-4-fluorophenyl)-1-methyl-(1R)-(3R-methyl-6-oxo-1,2,3,4,5,6-hexahydrophenanthridin-1-yl)urea;
3-(3-chloro-4-fluorophenyl)-1-methyl-(1R)-(3 S-methyl-6-oxo-1,2,3,4,5,6-hexahydrophenanthridin-1-yl)urea;
3-(3-chloro-4-fluorophenyl)-1-methyl-(1S)-(3R-methyl-6-oxo-1,2,3,4,5,6-hexahydrophenanthridin-1-yl)urea;
3-(3-chloro-4-fluorophenyl)-1-methyl-(1S)-(3S-methyl-6-oxo-1,2,3,4,5,6-hexahydrophenanthridin-1-yl)urea;
(R)-3-(3-chloro-4-fluorophenyl)-1-(3,3-dimethyl-6-oxo-1,2,3,4,5,6-hexahydrophenanthridin-1-yl)-1-methylurea;
(S)-3-(3-chloro-4-fluorophenyl)-1-(3,3-dimethyl-6-oxo-1,2,3,4,5,6-hexahydrophenanthridin-1-yl)-1-methylurea;
(R)-3-(3-chloro-4-fluorophenyl)-1-(7,8-difluoro-6-oxo-1,2,3,4,5,6-hexahydrophenanthridin-1-yl)-1-methylurea;
(S)-3-(3-chloro-4-fluorophenyl)-1-(7,8-difluoro-6-oxo-1,2,3,4,5,6-hexahydrophenanthridin-1-yl)-1-methylurea;
(R)-3-(3-chloro-5-fluorophenyl)-1-methyl-1-(6-oxo-1,4,5,6-tetrahydro-2H-pyrano[3,4-c]isoquinolin-1-yl)urea;
(S)-3-(3-chloro-5-fluorophenyl)-1-methyl-1-(6-oxo-1,4,5,6-tetrahydro-2H-pyrano[3,4-c]isoquinolin-1-yl)urea;
(R)-3-(3-chloro-4-fluorophenyl)-1-isobutyl-1-(6-oxo-1,4,5,6-tetrahydro-2H-pyrano[3,4-c]isoquinolin-1-yl)urea;
(S)-3-(3-chloro-4-fluorophenyl)-1-isobutyl-1-(6-oxo-1,4,5,6-tetrahydro-2H-pyrano[3,4-c]isoquinolin-1-yl)urea;
(R)-3-(3-chloro-4-fluorophenyl)-1-methyl-1-(5-methyl-6-oxo-1,2,3,4,5,6-hexahydrophenanthridin-1-yl)urea;
(S)-3-(3-chloro-4-fluorophenyl)-1-methyl-1-(5-methyl-6-oxo-1,2,3,4,5,6-hexahydrophenanthridin-1-yl)urea;
(R)-3-(3,4-difluorophenyl)-1-(8-fluoro-6-oxo-1,2,3,4,5,6-hexahydrophenanthridin-1-yl)-1-methylurea;
(S)-3-(3,4-difluorophenyl)-1-(8-fluoro-6-oxo-1,2,3,4,5,6-hexahydrophenanthridin-1-yl)-1-methylurea;
(R)-1-(8-fluoro-6-oxo-1,2,3,4,5,6-hexahydrophenanthridin-1-yl)-1-methyl-3-(3,4,5-trifluorophenyl)urea;
(S)-1-(8-fluoro-6-oxo-1,2,3,4,5,6-hexahydrophenanthridin-1-yl)-1-methyl-3-(3,4,5-trifluorophenyl)urea;
(R)-3-(3-chloro-4-fluorophenyl)-1-(6-methoxy-1,2,3,4-tetrahydrophenanthridin-1-yl)-1-methylurea;
(S)-3-(3-chloro-4-fluorophenyl)-1-(6-methoxy-1,2,3,4-tetrahydrophenanthridin-1-yl)-1-methylurea;
(R)-3-(3-chloro-4-fluorophenyl)-1-(7,8-difluoro-6-oxo-1,4,5,6-tetrahydro-2H-pyrano[3,4-c]isoquinolin-1-yl)-1-methylurea;
(S)-3-(3-chloro-4-fluorophenyl)-1-(7,8-difluoro-6-oxo-1,4,5,6-tetrahydro-2H-pyrano[3,4-c]isoquinolin-1-yl)-1-methylurea;
(R)-3-(3-chloro-4-fluorophenyl)-1-(8-fluoro-6-oxo-1,4,5,6-tetrahydro-2H-pyrano[3,4-c]isoquinolin-1-yl)-1-isobutylurea;
(S)-3-(3-chloro-4-fluorophenyl)-1-(8-fluoro-6-oxo-1,4,5,6-tetrahydro-2H-pyrano[3,4-c]isoquinolin-1-yl)-1-isobutylurea;
(R)-3-(3-chloro-4-fluorophenyl)-1-ethyl-1-(8-fluoro-6-oxo-1,4,5,6-tetrahydro-2H-pyrano[3,4-c]isoquinolin-1-yl)urea;
(S)-3-(3-chloro-4-fluorophenyl)-1-ethyl-1-(8-fluoro-6-oxo-1,4,5,6-tetrahydro-2H-pyrano[3,4-c]isoquinolin-1-yl)urea;
(R)-3-(3-chloro-4-fluorophenyl)-1-(8,9-difluoro-6-oxo-1,4,5,6-tetrahydro-2H-pyrano[3,4-c]isoquinolin-1-yl)-1-ethylurea;
(S)-3-(3-chloro-4-fluorophenyl)-1-(8,9-difluoro-6-oxo-1,4,5,6-tetrahydro-2H-pyrano[3,4-c]isoquinolin-1-yl)-1-ethylurea;
(R)-3-(3-chloro-4-fluorophenyl)-1-methyl-1-(6-oxo-1,2,3,4,5,6,7,8,9,10-decahydrophenanthridin-1-yl)urea;
(S)-3-(3-chloro-4-fluorophenyl)-1-methyl-1-(6-oxo-1,2,3,4,5,6,7,8,9,10-decahydrophenanthridin-1-yl)urea;
(R)-3-(3,4-difluorophenyl)-1-methyl-1-(6-oxo-1,2,3,4,5,6,7,8,9,10-decahydrophenanthridin-1-yl)urea;
(S)-3-(3,4-difluorophenyl)-1-methyl-1-(6-oxo-1,2,3,4,5,6,7,8,9,10-decahydrophenanthridin-1-yl)urea;
(R)-3-(3,4-difluorophenyl)-1-ethyl-1-(6-oxo-1,2,3,4,5,6,7,8,9,10-decahydrophenanthridin-1-yl)urea;
(S)-3-(3,4-difluorophenyl)-1-ethyl-1-(6-oxo-1,2,3,4,5,6,7,8,9,10-decahydrophenanthridin-1-yl)urea;
(R)-3-(3-chloro-4-fluorophenyl)-1-ethyl-1-(6-oxo-1,2,3,4,5,6,7,8,9,10-decahydrophenanthridin-1-yl)urea;
(S)-3-(3-chloro-4-fluorophenyl)-1-ethyl-1-(6-oxo-1,2,3,4,5,6,7,8,9,10-decahydrophenanthridin-1-yl)urea;
(R)-3-(3-chloro-4-fluorophenyl)-1-(8-chloro-6-oxo-1,4,5,6-tetrahydro-2H-pyrano[3,4-c]isoquinolin-1-yl)-1-methylurea;
(S)-3-(3-chloro-4-fluorophenyl)-1-(8-chloro-6-oxo-1,4,5,6-tetrahydro-2H-pyrano[3,4-c]isoquinolin-1-yl)-1-methylurea;
(R)-3-(3-chloro-4-fluorophenyl)-1-(8-chloro-6-oxo-1,4,5,6-tetrahydro-2H-pyrano[3,4-c]isoquinolin-1-yl)-1-ethylurea;
(S)-3-(3-chloro-4-fluorophenyl)-1-(8-chloro-6-oxo-1,4,5,6-tetrahydro-2H-pyrano[3,4-c]isoquinolin-1-yl)-1-ethylurea;
(R)-3-(4-fluoro-3-methylphenyl)-1-(8-fluoro-6-oxo-1,4,5,6-tetrahydro-2H-pyrano[3,4-c]isoquinolin-1-yl)-1-methylurea;
(S)-3-(4-fluoro-3-methylphenyl)-1-(8-fluoro-6-oxo-1,4,5,6-tetrahydro-2H-pyrano[3,4-c]isoquinolin-1-yl)-1-methylurea;
(R)-1-(8-chloro-6-oxo-1,4,5,6-tetrahydro-2H-pyrano[3,4-c]isoquinolin-1-yl)-3-(4-fluoro-3-methylphenyl)-1-methylurea;
(S)-1-(8-chloro-6-oxo-1,4,5,6-tetrahydro-2H-pyrano[3,4-c]isoquinolin-1-yl)-3-(4-fluoro-3-methylphenyl)-1-methylurea;
(R)-1-(8-chloro-6-oxo-1,4,5,6-tetrahydro-2H-pyrano[3,4-c]isoquinolin-1-yl)-1-ethyl-3-(4-fluoro-3-methylphenyl)urea;
(S)-1-(8-chloro-6-oxo-1,4,5,6-tetrahydro-2H-pyrano[3,4-c]isoquinolin-1-yl)-1-ethyl-3-(4-fluoro-3-methylphenyl)urea;
(R)-3-(3-chloro-4-fluorophenyl)-1-methyl-1-(5-oxo-1,4,5,6,7,8,9,10-octahydro-2H-pyrano[3,4-c]quinolin-10-yl)urea;
(S)-3-(3-chloro-4-fluorophenyl)-1-methyl-1-(5-oxo-1,4,5,6,7,8,9,10-octahydro-2H-pyrano[3,4-c]quinolin-10-yl)urea;
(R)-3-(3,4-difluorophenyl)-1-methyl-1-(5-oxo-1,4,5,6,7,8,9,10-octahydro-2H-pyrano[3,4-c]quinolin-10-yl)urea;
(S)-3-(3,4-difluorophenyl)-1-methyl-1-(5-oxo-1,4,5,6,7,8,9,10-octahydro-2H-pyrano[3,4-c]quinolin-10-yl)urea;
(R)-3-(3-cyano-4-fluorophenyl)-1-(8-fluoro-6-oxo-1,4,5,6-tetrahydro-2H-pyrano[3,4-c]isoquinolin-1-yl)-1-methylurea;
(S)-3-(3-cyano-4-fluorophenyl)-1-(8-fluoro-6-oxo-1,4,5,6-tetrahydro-2H-pyrano[3,4-c]isoquinolin-1-yl)-1-methylurea;
(R)-1-(8-chloro-6-oxo-1,4,5,6-tetrahydro-2H-pyrano[3,4-c]isoquinolin-1-yl)-3-(3-cyano-4-fluorophenyl)-1-methylurea;
(S)-1-(8-chloro-6-oxo-1,4,5,6-tetrahydro-2H-pyrano[3,4-c]isoquinolin-1-yl)-3-(3-cyano-4-fluorophenyl)-1-methylurea;
(R)-1-(8-chloro-6-oxo-1,4,5,6-tetrahydro-2H-pyrano[3,4-c]isoquinolin-1-yl)-3-(3-cyano-4-fluorophenyl)-1-ethylurea;
(S)-1-(8-chloro-6-oxo-1,4,5,6-tetrahydro-2H-pyrano[3,4-c]isoquinolin-1-yl)-3-(3-cyano-4-fluorophenyl)-1-ethylurea;
(R)-1-(3-chloro-4-fluorophenyl)-3-(8-fluoro-6-oxo-1,4,5,6-tetrahydro-2H-pyrano[3,4-c]isoquinolin-1-yl)urea;
(S)-1-(3-chloro-4-fluorophenyl)-3-(8-fluoro-6-oxo-1,4,5,6-tetrahydro-2H-pyrano[3,4-c]isoquinolin-1-yl)urea;
(R)-1-(3-chloro-4-fluorophenyl)-3-(8,9-difluoro-6-oxo-1,4,5,6-tetrahydro-2H-pyrano[3,4-c]isoquinolin-1-yl)urea;
(S)-1-(3-chloro-4-fluorophenyl)-3-(8,9-difluoro-6-oxo-1,4,5,6-tetrahydro-2H-pyrano[3,4-c]isoquinolin-1-yl)urea;
(R)-1-(8,9-difluoro-6-oxo-1,4,5,6-tetrahydro-2H-pyrano[3,4-c]isoquinolin-1-yl)-3-(4-fluoro-3-methylphenyl)-1-methylurea;
(S)-1-(8,9-difluoro-6-oxo-1,4,5,6-tetrahydro-2H-pyrano[3,4-c]isoquinolin-1-yl)-3-(4-fluoro-3-methylphenyl)-1-methylurea;
(R)-3-(3-chloro-4-fluorophenyl)-1-(9-fluoro-6-oxo-1,4,5,6-tetrahydro-2H-pyrano[3,4-c]isoquinolin-1-yl)-1-methylurea;
(S)-3-(3-chloro-4-fluorophenyl)-1-(9-fluoro-6-oxo-1,4,5,6-tetrahydro-2H-pyrano[3,4-c]isoquinolin-1-yl)-1-methylurea;
(R)-3-(3-chloro-4-fluorophenyl)-1-ethyl-1-(9-fluoro-6-oxo-1,4,5,6-tetrahydro-2H-pyrano[3,4-c]isoquinolin-1-yl)urea;
(S)-3-(3-chloro-4-fluorophenyl)-1-ethyl-1-(9-fluoro-6-oxo-1,4,5,6-tetrahydro-2H-pyrano[3,4-c]isoquinolin-1-yl)urea;
(R)-3-(4-fluoro-3-methylphenyl)-1-(9-fluoro-6-oxo-1,4,5,6-tetrahydro-2H-pyrano[3,4-c]isoquinolin-1-yl)-1-methylurea;
(S)-3-(4-fluoro-3-methylphenyl)-1-(9-fluoro-6-oxo-1,4,5,6-tetrahydro-2H-pyrano[3,4-c]isoquinolin-1-yl)-1-methylurea;
(R)-1-ethyl-3-(4-fluoro-3-methylphenyl)-1-(9-fluoro-6-oxo-1,4,5,6-tetrahydro-2H-pyrano[3,4-c]isoquinolin-1-yl)urea;
(S)-1-ethyl-3-(4-fluoro-3-methylphenyl)-1-(9-fluoro-6-oxo-1,4,5,6-tetrahydro-2H-pyrano[3,4-c]isoquinolin-1-yl)urea;
(R)-3-(3-cyano-4-fluorophenyl)-1-(9-fluoro-6-oxo-1,4,5,6-tetrahydro-2H-pyrano[3,4-c]isoquinolin-1-yl)-1-methylurea;
(S)-3-(3-cyano-4-fluorophenyl)-1-(9-fluoro-6-oxo-1,4,5,6-tetrahydro-2H-pyrano[3,4-c]isoquinolin-1-yl)-1-methylurea;
(R)-3-(3-cyano-4-fluorophenyl)-1-ethyl-1-(9-fluoro-6-oxo-1,4,5,6-tetrahydro-2H-pyrano[3,4-c]isoquinolin-1-yl)urea;
(S)-3-(3-cyano-4-fluorophenyl)-1-ethyl-1-(9-fluoro-6-oxo-1,4,5,6-tetrahydro-2H-pyrano[3,4-c]isoquinolin-1-yl)urea;
(R)-3-(3-chloro-4-fluorophenyl)-1-methyl-1-(5-oxo-3,4,5,6,7,8,9,10-octahydro-1H-pyrano[4,3-c]quinolin-10-yl)urea;
(S)-3-(3-chloro-4-fluorophenyl)-1-methyl-1-(5-oxo-3,4,5,6,7,8,9,10-octahydro-1H-pyrano[4,3-c]quinolin-10-yl)urea;
(R)-3-(3,4-difluorophenyl)-1-methyl-1-(5-oxo-3,4,5,6,7,8,9,10-octahydro-1H-pyrano[4,3-c]quinolin-10-yl)urea;
(S)-3-(3,4-difluorophenyl)-1-methyl-1-(5-oxo-3,4,5,6,7,8,9,10-octahydro-1H-pyrano[4,3-c]quinolin-10-yl)urea;
(R)-3-(3-chloro-4-fluorophenyl)-1-methyl-1-(5-oxo-4,5,6,7,9,10-hexahydro-1H,3H-dipyrano[3,4-b:3′,4′-d]pyridin-10-yl)urea;
(S)-3-(3-chloro-4-fluorophenyl)-1-methyl-1-(5-oxo-4,5,6,7,9,10-hexahydro-1H,3H-dipyrano[3,4-b:3′,4′-d]pyridin-10-yl)urea;
(R)-3-(3,4-difluorophenyl)-1-methyl-1-(5-oxo-4,5,6,7,9,10-hexahydro-1H,3H-dipyrano[3,4-b:3′,4′-d]pyridin-10-yl)urea;
(S)-3-(3,4-difluorophenyl)-1-methyl-1-(5-oxo-4,5,6,7,9,10-hexahydro-1H,3H-dipyrano[3,4-b:3′,4′-d]pyridin-10-yl)urea;
(R)-3-(3-chloro-4-fluorophenyl)-1-(8-cyano-6-oxo-1,4,5,6-tetrahydro-2H-pyrano[3,4-c]isoquinolin-1-yl)-1-methylurea;
(S)-3-(3-chloro-4-fluorophenyl)-1-(8-cyano-6-oxo-1,4,5,6-tetrahydro-2H-pyrano[3,4-c]isoquinolin-1-yl)-1-methylurea;
(R)-3-(3-chloro-4-fluorophenyl)-1-(8-cyano-6-oxo-1,4,5,6-tetrahydro-2H-pyrano[3,4-c]isoquinolin-1-yl)-1-ethylurea;
(S)-3-(3-chloro-4-fluorophenyl)-1-(8-cyano-6-oxo-1,4,5,6-tetrahydro-2H-pyrano[3,4-c]isoquinolin-1-yl)-1-ethylurea;
(R)-3-(3-chloro-4-fluorophenyl)-1-methyl-1-(6-oxo-1,2,4,5,6,7,9,10-octahydrodipyrano[3,4-b:4′,3′-d]pyridin-1-yl)urea;
(S)-3-(3-chloro-4-fluorophenyl)-1-methyl-1-(6-oxo-1,2,4,5,6,7,9,10-octahydrodipyrano[3,4-b:4′,3′-d]pyridin-1-yl)urea;
(R)-3-(3,4-difluorophenyl)-1-methyl-1-(6-oxo-1,2,4,5,6,7,9,10-octahydrodipyrano[3,4-b:4′,3′-d]pyridin-1-yl)urea;
(S)-3-(3,4-difluorophenyl)-1-methyl-1-(6-oxo-1,2,4,5,6,7,9,10-octahydrodipyrano[3,4-b:4′,3′-d]pyridin-1-yl)urea;
(R)-3-(3-chloro-4-fluorophenyl)-1-methyl-1-(6-oxo-1,4,5,6-tetrahydro-2H-pyrano[3,4-b]thieno[3,2-d]pyridin-1-yl)urea;
(S)-3-(3-chloro-4-fluorophenyl)-1-methyl-1-(6-oxo-1,4,5,6-tetrahydro-2H-pyrano[3,4-b]thieno[3,2-d]pyridin-1-yl)urea;
(R)-3-(3-chloro-4-fluorophenyl)-1-methyl-1-(4-oxo-4,6,8,9-tetrahydro-5H-pyrano[3,4-b]thieno[2,3-d]pyridin-9-yl)urea;
(S)-3-(3-chloro-4-fluorophenyl)-1-methyl-1-(4-oxo-4,6,8,9-tetrahydro-5H-pyrano[3,4-b]thieno[2,3-d]pyridin-9-yl)urea;
(R)-3-(3,5-dichloro-4-fluorophenyl)-1-(8-fluoro-6-oxo-1,4,5,6-tetrahydro-2H-pyrano[3,4-c]isoquinolin-1-yl)-1-methylurea;
(S)-3-(3,5-dichloro-4-fluorophenyl)-1-(8-fluoro-6-oxo-1,4,5,6-tetrahydro-2H-pyrano[3,4-c]isoquinolin-1-yl)-1-methylurea;
(R)-3-(3,4-difluorophenyl)-1-(8-fluoro-6-oxo-1,4,5,6-tetrahydro-2H-pyrano[3,4-c]isoquinolin-1-yl)-1-isobutylurea;
(S)-3-(3,4-difluorophenyl)-1-(8-fluoro-6-oxo-1,4,5,6-tetrahydro-2H-pyrano[3,4-c]isoquinolin-1-yl)-1-isobutylurea;
(R)-1-(8,9-difluoro-6-oxo-1,4,5,6-tetrahydro-2H-pyrano[3,4-c]isoquinolin-1-yl)-1-methyl-3-phenylurea;
(S)-1-(8,9-difluoro-6-oxo-1,4,5,6-tetrahydro-2H-pyrano[3,4-c]isoquinolin-1-yl)-1-methyl-3-phenylurea;
(R)-1-(8,9-difluoro-6-oxo-1,4,5,6-tetrahydro-2H-pyrano[3,4-c]isoquinolin-1-yl)-3-(4-fluorophenyl)-1-methylurea;
(S)-1-(8,9-difluoro-6-oxo-1,4,5,6-tetrahydro-2H-pyrano[3,4-c]isoquinolin-1-yl)-3-(4-fluorophenyl)-1-methylurea;
(R)-3-(3-chloro-4-fluorophenyl)-1-methyl-1-(4-oxo-4,5,8,9-tetrahydro-6H-pyrano[3,4-b]thieno[3,4-d]pyridin-9-yl)urea;
(S)-3-(3-chloro-4-fluorophenyl)-1-methyl-1-(4-oxo-4,5,8,9-tetrahydro-6H-pyrano[3,4-b]thieno[3,4-d]pyridin-9-yl)urea;
(R)-3-(3-cyano-4-fluorophenyl)-1-(8,9-difluoro-6-oxo-1,4,5,6-tetrahydro-2H-pyrano[3,4-c]isoquinolin-1-yl)-1-methylurea;
(S)-3-(3-cyano-4-fluorophenyl)-1-(8,9-difluoro-6-oxo-1,4,5,6-tetrahydro-2H-pyrano[3,4-c]isoquinolin-1-yl)-1-methylurea;
(R)-3-(3-chloro-4,5-difluorophenyl)-1-(8-fluoro-6-oxo-1,4,5,6-tetrahydro-2H-pyrano[3,4-c]isoquinolin-1-yl)-1-methylurea;
(S)-3-(3-chloro-4,5-difluorophenyl)-1-(8-fluoro-6-oxo-1,4,5,6-tetrahydro-2H-pyrano[3,4-c]isoquinolin-1-yl)-1-methylurea;
(R)-3-(3-chloro-4-fluorophenyl)-1-(8-fluoro-6-oxo-1,4,5,6-tetrahydro-2H-pyrano[3,4-c]isoquinolin-1-yl)-1-(3-hydroxypropyl)urea;
(S)-3-(3-chloro-4-fluorophenyl)-1-(8-fluoro-6-oxo-1,4,5,6-tetrahydro-2H-pyrano[3,4-c]isoquinolin-1-yl)-1-(3-hydroxypropyl)urea;
(R)-3-(3-chloro-4-fluorophenyl)-1-(8-fluoro-6-oxo-1,4,5,6-tetrahydro-2H-pyrano[3,4-c]isoquinolin-1-yl)-1-(2-hydroxy-2-methylpropyl)urea;
(S)-3-(3-chloro-4-fluorophenyl)-1-(8-fluoro-6-oxo-1,4,5,6-tetrahydro-2H-pyrano[3,4-c]isoquinolin-1-yl)-1-(2-hydroxy-2-methylpropyl)urea;
(R)-1-(8-fluoro-6-oxo-1,4,5,6-tetrahydro-2H-pyrano[3,4-c]isoquinolin-1-yl)-1-isobutyl-3-(3,4,5-trifluorophenyl)urea;
(S)-1-(8-fluoro-6-oxo-1,4,5,6-tetrahydro-2H-pyrano[3,4-c]isoquinolin-1-yl)-1-isobutyl-3-(3,4,5-trifluorophenyl)urea;
(R)-3-(3-cyano-4-fluorophenyl)-1-(8-fluoro-6-oxo-1,4,5,6-tetrahydro-2H-pyrano[3,4-c]isoquinolin-1-yl)-1-isobutylurea;
(S)-3-(3-cyano-4-fluorophenyl)-1-(8-fluoro-6-oxo-1,4,5,6-tetrahydro-2H-pyrano[3,4-c]isoquinolin-1-yl)-1-isobutylurea;
(R)-1-(8,9-difluoro-6-oxo-1,4,5,6-tetrahydro-2H-pyrano[3,4-c]isoquinolin-1-yl)-3-(3,4-difluorophenyl)-1-methylurea;
(S)-1-(8,9-difluoro-6-oxo-1,4,5,6-tetrahydro-2H-pyrano[3,4-c]isoquinolin-1-yl)-3-(3,4-difluorophenyl)-1-methylurea;
(R)-3-(3-chlorophenyl)-1-(8,9-difluoro-6-oxo-1,4,5,6-tetrahydro-2H-pyrano[3,4-c]isoquinolin-1-yl)-1-methylurea;
(S)-3-(3-chlorophenyl)-1-(8,9-difluoro-6-oxo-1,4,5,6-tetrahydro-2H-pyrano[3,4-c]isoquinolin-1-yl)-1-methylurea;
(R)-3-(3-cyano-4-fluorophenyl)-1-(8,9-difluoro-6-oxo-1,4,5,6-tetrahydro-2H-pyrano[3,4-c]isoquinolin-1-yl)-1-isobutylurea;
(S)-3-(3-cyano-4-fluorophenyl)-1-(8,9-difluoro-6-oxo-1,4,5,6-tetrahydro-2H-pyrano[3,4-c]isoquinolin-1-yl)-1-isobutylurea;
(R)-3-(3-(difluoromethyl)-4-fluorophenyl)-1-(8-fluoro-6-oxo-1,4,5,6-tetrahydro-2H-pyrano[3,4-c]isoquinolin-1-yl)-1-methylurea;
(S)-3-(3-(difluoromethyl)-4-fluorophenyl)-1-(8-fluoro-6-oxo-1,4,5,6-tetrahydro-2H-pyrano[3,4-c]isoquinolin-1-yl)-1-methylurea;
(R)-1-(8,9-difluoro-6-oxo-1,4,5,6-tetrahydro-2H-pyrano[3,4-c]isoquinolin-1-yl)-3-(3-(difluoromethyl)-4-fluorophenyl)-1-methylurea;
(S)-1-(8,9-difluoro-6-oxo-1,4,5,6-tetrahydro-2H-pyrano[3,4-c]isoquinolin-1-yl)-3-(3-(difluoromethyl)-4-fluorophenyl)-1-methylurea;
(R)-3-(4-fluoro-3-methylphenyl)-1-(8-fluoro-6-oxo-1,4,5,6-tetrahydro-2H-pyrano[3,4-c]isoquinolin-1-yl)-1-isobutylurea;
(S)-3-(4-fluoro-3-methylphenyl)-1-(8-fluoro-6-oxo-1,4,5,6-tetrahydro-2H-pyrano[3,4-c]isoquinolin-1-yl)-1-isobutylurea;
(R)-1-(8,9-difluoro-6-oxo-1,4,5,6-tetrahydro-2H-pyrano[3,4-c]isoquinolin-1-yl)-1-methyl-3-(3,4,5-trifluorophenyl)urea;
(S)-1-(8,9-difluoro-6-oxo-1,4,5,6-tetrahydro-2H-pyrano[3,4-c]isoquinolin-1-yl)-1-methyl-3-(3,4,5-trifluorophenyl)urea;
(R)-1-(3-cyano-4-fluorophenyl)-3-(8,9-difluoro-6-oxo-1,4,5,6-tetrahydro-2H-pyrano[3,4-c]isoquinolin-1-yl)urea;
(S)-1-(3-cyano-4-fluorophenyl)-3-(8,9-difluoro-6-oxo-1,4,5,6-tetrahydro-2H-pyrano[3,4-c]isoquinolin-1-yl)urea;
(R)-2-(3-(3-chloro-4-fluorophenyl)-1-(8,9-difluoro-6-oxo-1,4,5,6-tetrahydro-2H-pyrano[3,4-c]isoquinolin-1-yl)ureido)ethane-1-sulfonamide;
(S)-2-(3-(3-chloro-4-fluorophenyl)-1-(8,9-difluoro-6-oxo-1,4,5,6-tetrahydro-2H-pyrano[3,4-c]isoquinolin-1-yl)ureido)ethane-1-sulfonamide;
(R)-3-(3-cyano-4-fluorophenyl)-1-(8,9-difluoro-6-oxo-1,4,5,6-tetrahydro-2H-pyrano[3,4-c]isoquinolin-1-yl)-1-ethylurea;
(S)-3-(3-cyano-4-fluorophenyl)-1-(8,9-difluoro-6-oxo-1,4,5,6-tetrahydro-2H-pyrano[3,4-c]isoquinolin-1-yl)-1-ethylurea;
(R)-3-(3-chloro-4-fluorophenyl)-1-(8,9-difluoro-6-oxo-1,4,5,6-tetrahydro-2H-pyrano[3,4-c]isoquinolin-1-yl)-1-(2-(methylsulfonyl)ethyl)urea;
(S)-3-(3-chloro-4-fluorophenyl)-1-(8,9-difluoro-6-oxo-1,4,5,6-tetrahydro-2H-pyrano[3,4-c]isoquinolin-1-yl)-1-(2-(methylsulfonyl)ethyl)urea;
(R)-3-(4-chloro-3-fluorophenyl)-1-(8,9-difluoro-6-oxo-1,4,5,6-tetrahydro-2H-pyrano[3,4-c]isoquinolin-1-yl)-1-methylurea;
(S)-3-(4-chloro-3-fluorophenyl)-1-(8,9-difluoro-6-oxo-1,4,5,6-tetrahydro-2H-pyrano[3,4-c]isoquinolin-1-yl)-1-methylurea;
(R)-3-(4-chloro-3-cyanophenyl)-1-(8,9-difluoro-6-oxo-1,4,5,6-tetrahydro-2H-pyrano[3,4-c]isoquinolin-1-yl)-1-methylurea;
(S)-3-(4-chloro-3-cyanophenyl)-1-(8,9-difluoro-6-oxo-1,4,5,6-tetrahydro-2H-pyrano[3,4-c]isoquinolin-1-yl)-1-methylurea;
(R)-3-(3,4-dichlorophenyl)-1-(8,9-difluoro-6-oxo-1,4,5,6-tetrahydro-2H-pyrano[3,4-c]isoquinolin-1-yl)-1-methylurea;
(S)-3-(3,4-dichlorophenyl)-1-(8,9-difluoro-6-oxo-1,4,5,6-tetrahydro-2H-pyrano[3,4-c]isoquinolin-1-yl)-1-methylurea;
(R)-1-(8,9-difluoro-6-oxo-1,2,3,4,5,6-hexahydrophenanthridin-1-yl)-1-methyl-3-(1-(trifluoromethyl)cyclopropyl)urea;
(S)-1-(8,9-difluoro-6-oxo-1,2,3,4,5,6-hexahydrophenanthridin-1-yl)-1-methyl-3-(1-(trifluoromethyl)cyclopropyl)urea;
(R)-1-(8,9-difluoro-6-oxo-1,4,5,6-tetrahydro-2H-pyrano[3,4-c]isoquinolin-1-yl)-1-methyl-3-(1-(trifluoromethyl)cyclopropyl)urea;
(S)-1-(8,9-difluoro-6-oxo-1,4,5,6-tetrahydro-2H-pyrano[3,4-c]isoquinolin-1-yl)-1-methyl-3-(1-(trifluoromethyl)cyclopropyl)urea;
(R)-3-(3-Chloro-4-fluorophenyl)-1-(8,9-difluoro-6-oxo-1,4,5,6-tetrahydro-2H-pyrano[3,4-c]isoquinolin-1-yl-1-d)-1-(methyl-d3)urea;
(S)-3-(3-Chloro-4-fluorophenyl)-1-(8,9-difluoro-6-oxo-1,4,5,6-tetrahydro-2H-pyrano[3,4-c]isoquinolin-1-yl-1-d)-1-(methyl-d3)urea;
(R)-3-(3-Chloro-4-methoxyphenyl)-1-(8,9-difluoro-6-oxo-1,4,5,6-tetrahydro-2H-pyrano[3,4-c]isoquinolin-1-yl)-1-methylurea;
(S)-3-(3-Chloro-4-methoxyphenyl)-1-(8,9-difluoro-6-oxo-1,4,5,6-tetrahydro-2H-pyrano[3,4-c]isoquinolin-1-yl)-1-methylurea;
(R)-3-(3-Chloro-4-hydroxyphenyl)-1-(8,9-difluoro-6-oxo-1,4,5,6-tetrahydro-2H-pyrano[3,4-c]isoquinolin-1-yl)-1-methylurea;
(S)-3-(3-Chloro-4-hydroxyphenyl)-1-(8,9-difluoro-6-oxo-1,4,5,6-tetrahydro-2H-pyrano[3,4-c]isoquinolin-1-yl)-1-methylurea;
(R)-3-(3-Chloro-4-fluorophenyl)-1-(8,9-difluoro-6-oxo-1,2,3,4,5,6-hexahydrobenzo[c][1,7]naphthyridin-1-yl)-1-methylurea;
(S)-3-(3-Chloro-4-fluorophenyl)-1-(8,9-difluoro-6-oxo-1,2,3,4,5,6-hexahydrobenzo[c][1,7]naphthyridin-1-yl)-1-methylurea;
3-(3-Chloro-4-fluorophenyl)-(1R)-(8,9-difluoro-4R-hydroxy-6-oxo-1,4,5,6-tetrahydro-2H-pyrano[3,4-c]isoquinolin-1-yl)-1-methylurea;
3-(3-Chloro-4-fluorophenyl)-(1R)-(8,9-difluoro-4S-hydroxy-6-oxo-1,4,5,6-tetrahydro-2H-pyrano[3,4-c]isoquinolin-1-yl)-1-methylurea;
3-(3-Chloro-4-fluorophenyl)-(1S)-(8,9-difluoro-4R-hydroxy-6-oxo-1,4,5,6-tetrahydro-2H-pyrano[3,4-c]isoquinolin-1-yl)-1-methylurea;
3-(3-Chloro-4-fluorophenyl)-(1S)-(8,9-difluoro-4S-hydroxy-6-oxo-1,4,5,6-tetrahydro-2H-pyrano[3,4-c]isoquinolin-1-yl)-1-methylurea;
(R)-3-(3-chloro-4-Fluorophenyl)-1-(8,9-difluoro-4,6-dioxo-1,4,5,6-tetrahydro-2H-pyrano[3,4-c]isoquinolin-1-yl)-1-methylurea;
(S)-3-(3-chloro-4-Fluorophenyl)-1-(8,9-difluoro-4,6-dioxo-1,4,5,6-tetrahydro-2H-pyrano[3,4-c]isoquinolin-1-yl)-1-methylurea;
(R)-3-(3-Chloro-4-fluorophenyl)-1-(8-fluoro-6-oxo-1,2,3,4,5,6-hexahydrobenzo[c][1,7]naphthyridin-1-yl)-1-methylurea;
(S)-3-(3-Chloro-4-fluorophenyl)-1-(8-fluoro-6-oxo-1,2,3,4,5,6-hexahydrobenzo[c][1,7]naphthyridin-1-yl)-1-methylurea;
(R)-3-(3-Chloro-4-fluorophenyl)-1-(8-fluoro-3-methyl-6-oxo-1,2,3,4,5,6-hexahydrobenzo[c][1,7]naphthyridin-1-yl)-1-methylurea;
(S)-3-(3-Chloro-4-fluorophenyl)-1-(8-fluoro-3-methyl-6-oxo-1,2,3,4,5,6-hexahydrobenzo[c][1,7]naphthyridin-1-yl)-1-methylurea;
(R)-1-(3-Acetyl-8-fluoro-6-oxo-1,2,3,4,5,6-hexahydrobenzo[c][1,7]naphthyridin-1-yl)-3-(3-chloro-4-fluorophenyl)-1-methylurea;
(S)-1-(3-Acetyl-8-fluoro-6-oxo-1,2,3,4,5,6-hexahydrobenzo[c][1,7]naphthyridin-1-yl)-3-(3-chloro-4-fluorophenyl)-1-methylurea;
(R)-1-(3-Acetyl-8,9-difluoro-6-oxo-1,2,3,4,5,6-hexahydrobenzo[c][1,7]naphthyridin-1-yl)-3-(3-chloro-4-fluorophenyl)-1-methylurea;
(S)-1-(3-Acetyl-8,9-difluoro-6-oxo-1,2,3,4,5,6-hexahydrobenzo[c][1,7]naphthyridin-1-yl)-3-(3-chloro-4-fluorophenyl)-1-methylurea;
(R)-3-(3-Cyano-4-fluorophenyl)-1-(8-fluoro-6-oxo-1,2,3,4,5,6-hexahydrobenzo[c][1,7]naphthyridin-1-yl)-1-methylurea;
(S)-3-(3-Cyano-4-fluorophenyl)-1-(8-fluoro-6-oxo-1,2,3,4,5,6-hexahydrobenzo[c][1,7]naphthyridin-1-yl)-1-methylurea;
(R)-3-(3-Cyano-4-fluorophenyl)-1-(8-fluoro-3-methyl-6-oxo-1,2,3,4,5,6-hexahydrobenzo[c][1,7]naphthyridin-1-yl)-1-methylurea;
(S)-3-(3-Cyano-4-fluorophenyl)-1-(8-fluoro-3-methyl-6-oxo-1,2,3,4,5,6-hexahydrobenzo[c][1,7]naphthyridin-1-yl)-1-methylurea;
(R)-1-(3-Acetyl-8-fluoro-6-oxo-1,2,3,4,5,6-hexahydrobenzo[c][1,7]naphthyridin-1-yl)-3-(3-cyano-4-fluorophenyl)-1-methylurea;
(S)-1-(3-Acetyl-8-fluoro-6-oxo-1,2,3,4,5,6-hexahydrobenzo[c][1,7]naphthyridin-1-yl)-3-(3-cyano-4-fluorophenyl)-1-methylurea;
(R)-3-(3-Chloro-4-fluorophenyl)-1-(8,9-difluoro-3-methyl-6-oxo-1,2,3,4,5,6-hexahydrobenzo[c][1,7]naphthyridin-1-yl)-1-methylurea;
(S)-3-(3-Chloro-4-fluorophenyl)-1-(8,9-difluoro-3-methyl-6-oxo-1,2,3,4,5,6-hexahydrobenzo[c][1,7]naphthyridin-1-yl)-1-methylurea;
(R)-3-(3-Chloro-4-fluorophenyl)-1-(8-fluoro-3-(2-hydroxyethyl)-6-oxo-1,2,3,4,5,6-hexahydrobenzo[c][1,7]naphthyridin-1-yl)-1-methylurea;
(S)-3-(3-Chloro-4-fluorophenyl)-1-(8-fluoro-3-(2-hydroxyethyl)-6-oxo-1,2,3,4,5,6-hexahydrobenzo[c][1,7]naphthyridin-1-yl)-1-methylurea;
(R)-3-(3-Cyano-4-fluorophenyl)-1-(8-fluoro-3-(2-hydroxyethyl)-6-oxo-1,2,3,4,5,6-hexahydrobenzo[c][1,7]naphthyridin-1-yl)-1-methylurea;
(S)-3-(3-Cyano-4-fluorophenyl)-1-(8-fluoro-3-(2-hydroxyethyl)-6-oxo-1,2,3,4,5,6-hexahydrobenzo[c][1,7]naphthyridin-1-yl)-1-methylurea;
(R)-3-(3-Cyano-4-fluorophenyl)-1-(8,9-difluoro-3-methyl-6-oxo-1,2,3,4,5,6-hexahydrobenzo[c][1,7]naphthyridin-1-yl)-1-methylurea;
(S)-3-(3-Cyano-4-fluorophenyl)-1-(8,9-difluoro-3-methyl-6-oxo-1,2,3,4,5,6-hexahydrobenzo[c][1,7]naphthyridin-1-yl)-1-methylurea;
(R)-3-(3-Cyano-4-fluorophenyl)-1-(8,9-difluoro-6-oxo-1,2,3,4,5,6-hexahydrobenzo[c][1,7]naphthyridin-1-yl)-1-methylurea;
(S)-3-(3-Cyano-4-fluorophenyl)-1-(8,9-difluoro-6-oxo-1,2,3,4,5,6-hexahydrobenzo[c][1,7]naphthyridin-1-yl)-1-methylurea;
(R)-1-(3-Acetyl-8,9-difluoro-6-oxo-1,2,3,4,5,6-hexahydrobenzo[c][1,7]naphthyridin-1-yl)-3-(3-cyano-4-fluorophenyl)-1-methylurea;
(S)-1-(3-Acetyl-8,9-difluoro-6-oxo-1,2,3,4,5,6-hexahydrobenzo[c][1,7]naphthyridin-1-yl)-3-(3-cyano-4-fluorophenyl)-1-methylurea;
(R)-3-(3-Chloro-4-fluorophenyl)-1-(8,9-difluoro-3-(2-hydroxyethyl)-6-oxo-1,2,3,4,5,6-hexahydrobenzo[c][1,7]naphthyridin-1-yl)-1-methylurea;
(S)-3-(3-Chloro-4-fluorophenyl)-1-(8,9-difluoro-3-(2-hydroxyethyl)-6-oxo-1,2,3,4,5,6-hexahydrobenzo[c][1,7]naphthyridin-1-yl)-1-methylurea;
(R)-3-(3-Cyano-4-fluorophenyl)-1-(8,9-difluoro-3-(2-hydroxyethyl)-6-oxo-1,2,3,4,5,6-hexahydrobenzo[c][1,7]naphthyridine-1-yl)-1-methylurea;
(S)-3-(3-Cyano-4-fluorophenyl)-1-(8,9-difluoro-3-(2-hydroxyethyl)-6-oxo-1,2,3,4,5,6-hexahydrobenzo[c][1,7]naphthyridine-1-yl)-1-methylurea;
(R)-3-(3-Chloro-4-fluorophenyl)-1-(8-fluoro-6-oxo-1,4,5,6-tetrahydro-2H-thiopyrano[3,4-c]isoquinolin-1-yl)-1-methylurea;
(S)-3-(3-Chloro-4-fluorophenyl)-1-(8-fluoro-6-oxo-1,4,5,6-tetrahydro-2H-thiopyrano[3,4-c]isoquinolin-1-yl)-1-methylurea;
(R)-3-(3-Cyano-4-fluorophenyl)-1-(8-fluoro-6-oxo-1,4,5,6-tetrahydro-2H-thiopyrano[3,4-c]isoquinolin-1-yl)-1-methylurea;
(S)-3-(3-Cyano-4-fluorophenyl)-1-(8-fluoro-6-oxo-1,4,5,6-tetrahydro-2H-thiopyrano[3,4-c]isoquinolin-1-yl)-1-methylurea;
(R)-3-(3-Chloro-4-fluorophenyl)-1-(8,9-difluoro-6-oxo-1,4,5,6-tetrahydro-2H-thiopyrano[3,4-c]isoquinolin-1-yl)-1-methylurea;
(S)-3-(3-Chloro-4-fluorophenyl)-1-(8,9-difluoro-6-oxo-1,4,5,6-tetrahydro-2H-thiopyrano[3,4-c]isoquinolin-1-yl)-1-methylurea;
(R)-3-(3-Cyano-4-fluorophenyl)-1-(8,9-difluoro-6-oxo-1,4,5,6-tetrahydro-2H-thiopyrano[3,4-c]isoquinolin-1-yl)-1-methylurea;
(S)-3-(3-Cyano-4-fluorophenyl)-1-(8,9-difluoro-6-oxo-1,4,5,6-tetrahydro-2H-thiopyrano[3,4-c]isoquinolin-1-yl)-1-methylurea;
(R)-1-(8,9-Difluoro-6-oxo-1,2,3,4,5,6-hexahydrobenzo[c][1,7]naphthyridin-1-yl)-3-(3,4-difluorophenyl)-1-methylurea;
(S)-1-(8,9-Difluoro-6-oxo-1,2,3,4,5,6-hexahydrobenzo[c][1,7]naphthyridin-1-yl)-3-(3,4-difluorophenyl)-1-methylurea;
3-(3-Chloro-4-fluorophenyl)-(1R)-(8-fluoro-3R-oxido-6-oxo-1,4,5,6-tetrahydro-2H-thiopyrano[3,4-c]isoquinolin-1-yl)-1-methylurea;
3-(3-Chloro-4-fluorophenyl)-(1R)-(8-fluoro-3 S-oxido-6-oxo-1,4,5,6-tetrahydro-2H-thiopyrano[3,4-c]isoquinolin-1-yl)-1-methylurea;
3-(3-Chloro-4-fluorophenyl)-(1S)-(8-fluoro-3R-oxido-6-oxo-1,4,5,6-tetrahydro-2H-thiopyrano[3,4-c]isoquinolin-1-yl)-1-methylurea;
3-(3-Chloro-4-fluorophenyl)-(1S)-(8-fluoro-3S-oxido-6-oxo-1,4,5,6-tetrahydro-2H-thiopyrano[3,4-c]isoquinolin-1-yl)-1-methylurea;
(R)-3-(3-Chloro-4-fluorophenyl)-1-(8-fluoro-3,3-dioxido-6-oxo-1,4,5,6-tetrahydro-2H-thiopyrano[3,4-c]isoquinolin-1-yl)-1-methylurea;
(S)-3-(3-Chloro-4-fluorophenyl)-1-(8-fluoro-3,3-dioxido-6-oxo-1,4,5,6-tetrahydro-2H-thiopyrano[3,4-c]isoquinolin-1-yl)-1-methylurea;
3-(3-Cyano-4-fluorophenyl)-(1R)-(8-fluoro-3R-oxido-6-oxo-1,4,5,6-tetrahydro-2H-thiopyrano[3,4-c]isoquinolin-1-yl)-1-methylurea;
3-(3-Cyano-4-fluorophenyl)-(1R)-(8-fluoro-3 S-oxido-6-oxo-1,4,5,6-tetrahydro-2H-thiopyrano[3,4-c]isoquinolin-1-yl)-1-methylurea;
3-(3-Cyano-4-fluorophenyl)-(1S)-(8-fluoro-3R-oxido-6-oxo-1,4,5,6-tetrahydro-2H-thiopyrano[3,4-c]isoquinolin-1-yl)-1-methylurea;
3-(3-Cyano-4-fluorophenyl)-(1S)-(8-fluoro-3S-oxido-6-oxo-1,4,5,6-tetrahydro-2H-thiopyrano[3,4-c]isoquinolin-1-yl)-1-methylurea;
(R)-3-(3-Chloro-4-fluorophenyl)-1-(8,9-difluoro-3,3-dioxido-6-oxo-1,4,5,6-tetrahydro-2H-thiopyrano[3,4-c]isoquinolin-1-yl)-1-methylurea;
(S)-3-(3-Chloro-4-fluorophenyl)-1-(8,9-difluoro-3,3-dioxido-6-oxo-1,4,5,6-tetrahydro-2H-thiopyrano[3,4-c]isoquinolin-1-yl)-1-methylurea;
(R)-3-(3-Cyano-4-fluorophenyl)-1-(8,9-difluoro-3,3-dioxido-6-oxo-1,4,5,6-tetrahydro-2H-thiopyrano [3,4-c]isoquinolin-1-yl)-1-methylurea;
(S)-3-(3-Cyano-4-fluorophenyl)-1-(8,9-difluoro-3,3-dioxido-6-oxo-1,4,5,6-tetrahydro-2H-thiopyrano[3,4-c]isoquinolin-1-yl)-1-methylurea;
3-(3-Chloro-4-fluorophenyl)-(1R)-(8,9-difluoro-3R-oxido-6-oxo-1,4,5,6-tetrahydro-2H-thiopyrano[3,4-c]isoquinolin-1-yl)-1-methylurea;
3-(3-Chloro-4-fluorophenyl)-(1R)-(8,9-difluoro-3 S-oxido-6-oxo-1,4,5,6-tetrahydro-2H-thiopyrano[3,4-c]isoquinolin-1-yl)-1-methylurea;
3-(3-Chloro-4-fluorophenyl)-(1S)-(8,9-difluoro-3R-oxido-6-oxo-1,4,5,6-tetrahydro-2H-thiopyrano[3,4-c]isoquinolin-1-yl)-1-methylurea;
3-(3-Chloro-4-fluorophenyl)-(1S)-(8,9-difluoro-3S-oxido-6-oxo-1,4,5,6-tetrahydro-2H-thiopyrano[3,4-c]isoquinolin-1-yl)-1-methylurea;
3-(3-Cyano-4-fluorophenyl)-(1R)-(8,9-difluoro-3R-oxido-6-oxo-1,4,5,6-tetrahydro-2H-thiopyrano[3,4-c]isoquinolin-1-yl)-1-methylurea;
3-(3-Cyano-4-fluorophenyl)-(1R)-(8,9-difluoro-3 S-oxido-6-oxo-1,4,5,6-tetrahydro-2H-thiopyrano[3,4-c]isoquinolin-1-yl)-1-methylurea;
3-(3-Cyano-4-fluorophenyl)-(1S)-(8,9-difluoro-3R-oxido-6-oxo-1,4,5,6-tetrahydro-2H-thiopyrano[3,4-c]isoquinolin-1-yl)-1-methylurea;
3-(3-Cyano-4-fluorophenyl)-(1S)-(8,9-difluoro-3S-oxido-6-oxo-1,4,5,6-tetrahydro-2H-thiopyrano[3,4-c]isoquinolin-1-yl)-1-methylurea;
(R)-3-(3-Cyano-4-fluorophenyl)-1-(8-fluoro-3,3-dioxido-6-oxo-1,4,5,6-tetrahydro-2H-thiopyrano[3,4-c]isoquinolin-1-yl)-1-methylurea;
(S)-3-(3-Cyano-4-fluorophenyl)-1-(8-fluoro-3,3-dioxido-6-oxo-1,4,5,6-tetrahydro-2H-thiopyrano[3,4-c]isoquinolin-1-yl)-1-methylurea;
(R)-1-(8,9-difluoro-6-oxo-1,2,3,4,5,6-hexahydrobenzo[c][1,7]naphthyridin-1-yl)-3-(3-(difluoromethyl)-4-fluorophenyl)-1-methylurea;
(S)-1-(8,9-difluoro-6-oxo-1,2,3,4,5,6-hexahydrobenzo[c][1,7]naphthyridin-1-yl)-3-(3-(difluoromethyl)-4-fluorophenyl)-1-methylurea;
(R)—N-(8,9-difluoro-6-oxo-1,4,5,6-tetrahydro-2H-pyrano[3,4-c]isoquinolin-1-yl)-N-methylisoindoline-2-carboxamide;
(S)—N-(8,9-difluoro-6-oxo-1,4,5,6-tetrahydro-2H-pyrano[3,4-c]isoquinolin-1-yl)-N-methylisoindoline-2-carboxamide;
(R)-5-chloro-N-(8,9-difluoro-6-oxo-1,4,5,6-tetrahydro-2H-pyrano[3,4-c]isoquinolin-1-yl)-N-methylisoindoline-2-carboxamide;
(S)-5-chloro-N-(8,9-difluoro-6-oxo-1,4,5,6-tetrahydro-2H-pyrano[3,4-c]isoquinolin-1-yl)-N-methylisoindoline-2-carboxamide;
(R)-5-bromo-N-(8,9-difluoro-6-oxo-1,4,5,6-tetrahydro-2H-pyrano[3,4-c]isoquinolin-1-yl)-N-methylisoindoline-2-carboxamide;
(S)-5-bromo-N-(8,9-difluoro-6-oxo-1,4,5,6-tetrahydro-2H-pyrano[3,4-c]isoquinolin-1-yl)-N-methylisoindoline-2-carboxamide;
(R)-5-fluoro-N-(8,9-difluoro-6-oxo-1,4,5,6-tetrahydro-2H-pyrano[3,4-c]isoquinolin-1-yl)-N-methylisoindoline-2-carboxamide;
(S)-5-fluoro-N-(8,9-difluoro-6-oxo-1,4,5,6-tetrahydro-2H-pyrano[3,4-c]isoquinolin-1-yl)-N-methylisoindoline-2-carboxamide;
(R)—N-(8,9-difluoro-6-oxo-1,2,3,4,5,6-hexahydrobenzo[c][1,7]naphthyridin-1-yl)-N-methylisoindoline-2-carboxamide;
(S)—N-(8,9-difluoro-6-oxo-1,2,3,4,5,6-hexahydrobenzo[c][1,7]naphthyridin-1-yl)-N-methylisoindoline-2-carboxamide;
(R)—N-(8,9-difluoro-6-oxo-1,2,3,4,5,6-hexahydrobenzo[c][1,7]naphthyridin-1-yl)-5-fluoro-N-methylisoindoline-2-carboxamide;
(S)—N-(8,9-difluoro-6-oxo-1,2,3,4,5,6-hexahydrobenzo[c][1,7]naphthyridin-1-yl)-5-fluoro-N-methylisoindoline-2-carboxamide;
(R)—N-(8,9-difluoro-6-oxo-1,2,3,4,5,6-hexahydrobenzo[c][1,7]naphthyridin-1-yl)-5-chloro-N-methylisoindoline-2-carboxamide;
(S)—N-(8,9-difluoro-6-oxo-1,2,3,4,5,6-hexahydrobenzo[c][1,7]naphthyridin-1-yl)-5-chloro-N-methylisoindoline-2-carboxamide;
(R)—N-(8,9-difluoro-6-oxo-1,2,3,4,5,6-hexahydrobenzo[c][1,7]naphthyridin-1-yl)-5-bromo-N-methylisoindoline-2-carboxamide;
(S)—N-(8,9-difluoro-6-oxo-1,2,3,4,5,6-hexahydrobenzo[c][1,7]naphthyridin-1-yl)-5-bromo-N-methylisoindoline-2-carboxamide;
(R)—N-(8,9-difluoro-6-oxo-1,4,5,6-tetrahydro-2H-pyrano[3,4-c]isoquinolin-1-yl)-N-methyl-5-(trifluoromethyl)isoindoline-2-carboxamide;
(S)—N-(8,9-difluoro-6-oxo-1,4,5,6-tetrahydro-2H-pyrano[3,4-c]isoquinolin-1-yl)-N-methyl-5-(trifluoromethyl)isoindoline-2-carboxamide;
(R)—N-(8,9-difluoro-6-oxo-1,2,3,4,5,6-hexahydrobenzo[c][1,7]naphthyridin-1-yl)-N-methyl-5-(trifluoromethyl)isoindoline-2-carboxamide;
(S)—N-(8,9-difluoro-6-oxo-1,2,3,4,5,6-hexahydrobenzo[c][1,7]naphthyridin-1-yl)-N-methyl-5-(trifluoromethyl)isoindoline-2-carboxamide;
(R)-1-(8,9-difluoro-5-methyl-6-oxo-1,4,5,6-tetrahydro-2H-pyrano[3,4-c]isoquinolin-1-yl)-3-(3-(difluoromethyl)-4-fluorophenyl)-1-methylurea;
(S)-1-(8,9-difluoro-5-methyl-6-oxo-1,4,5,6-tetrahydro-2H-pyrano[3,4-c]isoquinolin-1-yl)-3-(3-(difluoromethyl)-4-fluorophenyl)-1-methylurea;
(R)-1-(8,9-difluoro-6-methoxy-1,4-dihydro-2H-pyrano[3,4-c]isoquinolin-1-yl)-3-(3-(difluoromethyl)-4-fluorophenyl)-1-methylurea;
(S)-1-(8,9-difluoro-6-methoxy-1,4-dihydro-2H-pyrano[3,4-c]isoquinolin-1-yl)-3-(3-(difluoromethyl)-4-fluorophenyl)-1-methylurea;
(R)-1-(8,9-difluoro-6-(methylamino)-1,4-dihydro-2H-pyrano[3,4-c]isoquinolin-1-yl)-3-(3-(difluoromethyl)-4-fluorophenyl)-1-methylurea
(S)-1-(8,9-difluoro-6-(methylamino)-1,4-dihydro-2H-pyrano[3,4-c]isoquinolin-1-yl)-3-(3-(difluoromethyl)-4-fluorophenyl)-1-methylurea;
(R)-1-(8,9-difluoro-6-((2-hydroxyethyl)amino)-1,4-dihydro-2H-pyrano[3,4-c]isoquinolin-1-yl)-3-(3-(difluoromethyl)-4-fluorophenyl)-1-methylurea;
(S)-1-(8,9-difluoro-6-((2-hydroxyethyl)amino)-1,4-dihydro-2H-pyrano[3,4-c]isoquinolin-1-yl)-3-(3-(difluoromethyl)-4-fluorophenyl)-1-methylurea;
(R)-1-(8,9-difluoro-6-((2-aminoethyl)amino)-1,4-dihydro-2H-pyrano[3,4-c]isoquinolin-1-yl)-3-(3-(difluoromethyl)-4-fluorophenyl)-1-methylurea
(S)-1-(8,9-difluoro-6-((2-aminoethyl)amino)-1,4-dihydro-2H-pyrano[3,4-c]isoquinolin-1-yl)-3-(3-(difluoromethyl)-4-fluorophenyl)-1-methylurea;
(R)-3-(3-chloro-4-fluorophenyl)-1-(8,9-difluoro-6-((2-aminoethyl)amino)-1,4-dihydro-2H-pyrano[3,4-c]isoquinolin-1-yl)-1-methylurea;
(S)-3-(3-chloro-4-fluorophenyl)-1-(8,9-difluoro-6-((2-aminoethyl)amino)-1,4-dihydro-2H-pyrano[3,4-c]isoquinolin-1-yl)-1-methylurea;
(R)-3-(3-chloro-4-fluorophenyl)-1-(8,9-difluoro-6-(methylamino)-1,4-dihydro-2H-pyrano[3,4-c]isoquinolin-1-yl)-1-methylurea;
(S)-3-(3-chloro-4-fluorophenyl)-1-(8,9-difluoro-6-(methylamino)-1,4-dihydro-2H-pyrano[3,4-c]isoquinolin-1-yl)-1-methylurea;
(R)-1-(8,9-difluoro-5-methyl-6-oxo-1,4,5,6-tetrahydro-2H-pyrano[3,4-c]isoquinolin-1-yl)-3-(3-chloro-4-fluorophenyl)-1-methylurea;
(S)-1-(8,9-difluoro-5-methyl-6-oxo-1,4,5,6-tetrahydro-2H-pyrano[3,4-c]isoquinolin-1-yl)-3-(3-chloro-4-fluorophenyl)-1-methylurea;
(R)-3-(3-chloro-4-fluorophenyl)-1-(8,9-difluoro-6-methoxy-1,4-dihydro-2H-pyrano[3,4-c]isoquinolin-1-yl)-1-methylurea;
(S)-3-(3-chloro-4-fluorophenyl)-1-(8,9-difluoro-6-methoxy-1,4-dihydro-2H-pyrano[3,4-c]isoquinolin-1-yl)-1-methylurea;
(R)-3-(3-chloro-4-fluorophenyl)-1-(8,9-difluoro-6-((2-hydroxyethyl)amino)-1,4-dihydro-2H-pyrano[3,4-c]isoquinolin-1-yl)-1-methylurea;
(S)-3-(3-chloro-4-fluorophenyl)-1-(8,9-difluoro-6-((2-hydroxyethyl)amino)-1,4-dihydro-2H-pyrano[3,4-c]isoquinolin-1-yl)-1-methylurea;
(R)-1-(8,9-difluoro-5-(2-hydroxyethyl)-6-oxo-1,4,5,6-tetrahydro-2H-pyrano[3,4-c]isoquinolin-1-yl)-3-(3-(difluoromethyl)-4-fluorophenyl)-1-methylurea;
(S)-1-(8,9-difluoro-5-(2-hydroxyethyl)-6-oxo-1,4,5,6-tetrahydro-2H-pyrano[3,4-c]isoquinolin-1-yl)-3-(3-(difluoromethyl)-4-fluorophenyl)-1-methylurea;
(R)-1-(6-(2-aminoethoxy)-8,9-difluoro-1,4-dihydro-2H-pyrano[3,4-c]isoquinolin-1-yl)-3-(3-(difluoromethyl)-4-fluorophenyl)-1-methylurea;
(S)-1-(6-(2-aminoethoxy)-8,9-difluoro-1,4-dihydro-2H-pyrano[3,4-c]isoquinolin-1-yl)-3-(3-(difluoromethyl)-4-fluorophenyl)-1-methylurea;
(R)-1-(5-(2-aminoethyl)-8,9-difluoro-6-oxo-1,4,5,6-tetrahydro-2H-pyrano[3,4-c]isoquinolin-1-yl)-3-(3-(difluoromethyl)-4-fluorophenyl)-1-methylurea;
(S)-1-(5-(2-aminoethyl)-8,9-difluoro-6-oxo-1,4,5,6-tetrahydro-2H-pyrano[3,4-c]isoquinolin-1-yl)-3-(3-(difluoromethyl)-4-fluorophenyl)-1-methylurea;
(R)-3-(3-chloro-4-fluorophenyl)-1-(8,9-difluoro-5-(2-hydroxyethyl)-6-oxo-1,4,5,6-tetrahydro-2H-pyrano[3,4-c]isoquinolin-1-yl)-1-methylurea;
(S)-3-(3-chloro-4-fluorophenyl)-1-(8,9-difluoro-5-(2-hydroxyethyl)-6-oxo-1,4,5,6-tetrahydro-2H-pyrano[3,4-c]isoquinolin-1-yl)-1-methylurea;
(R)-3-(3-chloro-4-fluorophenyl)-1-(8,9-difluoro-6-(2-hydroxyethoxy)-1,4-dihydro-2H-pyrano[3,4-c]isoquinolin-1-yl)-1-methylurea;
(S)-3-(3-chloro-4-fluorophenyl)-1-(8,9-difluoro-6-(2-hydroxyethoxy)-1,4-dihydro-2H-pyrano[3,4-c]isoquinolin-1-yl)-1-methylurea;
(R)-1-(8,9-difluoro-6-(2-hydroxyethoxy)-1,4-dihydro-2H-pyrano[3,4-c]isoquinolin-1-yl)-3-(3-(difluoromethyl)-4-fluorophenyl)-1-methylurea;
(S)-1-(8,9-difluoro-6-(2-hydroxyethoxy)-1,4-dihydro-2H-pyrano[3,4-c]isoquinolin-1-yl)-3-(3-(difluoromethyl)-4-fluorophenyl)-1-methylurea;
(R)-1-(5-(2-aminoethyl)-8,9-difluoro-6-oxo-1,4,5,6-tetrahydro-2H-pyrano[3,4-c]isoquinolin-1-yl)-3-(3-chloro-4-fluorophenyl)-1-methylurea;
(S)-1-(5-(2-aminoethyl)-8,9-difluoro-6-oxo-1,4,5,6-tetrahydro-2H-pyrano[3,4-c]isoquinolin-1-yl)-3-(3-chloro-4-fluorophenyl)-1-methylurea;
(R)-1-(6-(2-aminoethoxy)-8,9-difluoro-1,4-dihydro-2H-pyrano[3,4-c]isoquinolin-1-yl)-3-(3-chloro-4-fluorophenyl)-1-methylurea;
(S)-1-(6-(2-aminoethoxy)-8,9-difluoro-1,4-dihydro-2H-pyrano[3,4-c]isoquinolin-1-yl)-3-(3-chloro-4-fluorophenyl)-1-methylurea;
or a salt, solvate, prodrug, isotopically labelled, stereoisomer, any mixture of stereoisomers, tautomer, and/or any mixture of tautomers thereof.

20. A pharmaceutical composition comprising at least one compound of claim 1 and a pharmaceutically acceptable carrier, optionally wherein the pharmaceutical composition further comprises at least one additional agent useful for treating hepatitis infection.

21. (canceled)

22. The pharmaceutical composition of claim 20, wherein the at least one additional agent comprises at least one selected from the group consisting of reverse transcriptase inhibitor; capsid inhibitor; cccDNA formation inhibitor; RNA destabilizer; oligomeric nucleotide targeted against the HBV genome; immunostimulator; and GalNAc-siRNA conjugate targeted against an HBV gene transcript.

23. The pharmaceutical composition of claim 22, wherein the immunostimulator is a checkpoint inhibitor, and wherein the checkpoint inhibitor is optionally a PD-L1 inhibitor.

24. (canceled)

25. A method of treating, ameliorating, or preventing hepatitis B virus (HBV) infection in a subject, optionally wherein the subject is further infected with hepatitis D virus, the method comprising administering to the subject in need thereof a therapeutically effective amount of at least one compound of claim 1.

26. (canceled)

27. The method of claim 25, wherein the at least one compound is administered to the subject in a pharmaceutically acceptable composition.

28. The method of claim 25, wherein the subject is further administered at least one additional agent useful for treating, ameliorating, and/or preventing the HBV infection, wherein the at least one compound and the at least one agent are optionally co-administered, and wherein the optionally co-administered at least one compound and at least one additional agent are optionally coformulated.

29. The method of claim 28, wherein the at least one additional agent comprises at least one selected from the group consisting of reverse transcriptase inhibitor; capsid inhibitor; cccDNA formation inhibitor; RNA destabilizer; oligomeric nucleotide targeted against the HBV genome; immunostimulator; and GalNAc-siRNA conjugate targeted against an HBV gene transcript.

30. The method of claim 29, wherein the immunostimulator is a checkpoint inhibitor, and wherein the checkpoint inhibitor is optionally a PD-L1 inhibitor.

31. (canceled)

32. (canceled)

33. (canceled)

34. A method of inhibiting expression or function of a viral capsid protein directly or indirectly in a hepatitis B virus-infected subject, optionally wherein the subject is further infected with hepatitis D virus (HDV), the method comprising administering to the subject in need thereof a therapeutically effective amount of at least one compound of claim 1.

35. (canceled)

36. The method of claim 34, wherein the at least one compound is administered to the subject in a pharmaceutically acceptable composition.

37. The method of claim 34, wherein the subject is further administered at least one additional agent useful for treating, ameliorating, and/or preventing the hepatitis B viral infection, wherein the at least one compound and the at least one agent are optionally co-administered, and wherein the optionally co-administered at least one compound and at least one additional agent are optionally coformulated.

38. The method of claim 37, wherein the at least one additional agent comprises at least one selected from the group consisting of reverse transcriptase inhibitor; capsid inhibitor; cccDNA formation inhibitor; RNA destabilizer; oligomeric nucleotide targeted against the HBV genome; immunostimulator; and GalNAc-siRNA conjugate targeted against an HBV gene transcript.

39. The method of claim 38, wherein the immunostimulator is a checkpoint inhibitor, wherein the checkpoint inhibitor is optionally a PD-L1 inhibitor.

40. (canceled)

41. (canceled)

42. (canceled)

43. The method of claim 25, wherein the subject is a mammal, optionally the mammal being a human.

44. (canceled)
